SEQUENCE LISTING (283 sequences)

SEQ ID NO: 1 - DNA, 565 bp, Homo sapiens
  unsure (20)
  unsure (49)
  unsure (58)
  unsure (144)
  unsure (178)
  unsure (215)
  unsure (218)
  unsure (228)
  unsure (239)
  unsure (541)
gattgtcagtttccaaaaangaggaggatttgatattcacttggcccgnggtgatgcntttgagggtggccgcgtccatctggtcagaaaagacaatctttttgttgtcaagcttgaggtgtggcaggcttgagatctggccanacacttgagtgacaatgacatccactttgctttntctccacaggtgtccactcccaggtccaactgcaganttngaattcggcnttcatggcctntgaatagagacttctggactctatagaacccactgcctcctgatgaagtccctactgttcacccttgcagtttttatgctcctggcccaattggtctcaggtaattggtatgtgaaaaagtgtctaaacgacgttggaatttgcaagaagaagtgcaaacctgaagagatgcatacaaagactacaagaatttcaacagtaacagcaacaacagtcaacaacaactttgatgatgactactgcttcgatgtcttcgatggctcctacccgtttctcccactggtggaacattcccagcctcngtctcctgctctaggatccccgac

SEQ ID NO: 2 - protein, 94 aa, Homo sapiens
  UNSURE (87)
MKSLLFTLAVFMLLAQLVSGNWYVKKCLNDVGICKKKCKPEEMHTKTTRISTVTATTVNNNFDDDYCFDVFDGSYPFLPLVEHSQPXSPALGSP

SEQ ID NO: 3 - DNA, 92 bp, Homo sapiens
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 2949 bp, Homo sapiens
acctgctggaacgctcggacgtccttgcgggccctgtccatgtgaggtccaggtcatctcccgccaccgcagacaagcgccaggatgtgcttttggggcaagacagtggctttggtcttgtgaaggatccatgttatttggctggtcctggatctaggtcactcagttgttcagaaagaggccaagaagagatgctgccgctcttccaccatctcacccctcgttggggtggttcaggctgcaaagccattggtgattcctccgttcctagtgaatgtcctggaaccctggaccatcagaggcaagccagtaggacaccctgccccaggccaccactggcaggaacgcaagggctggtcacagacaccagggctgcacccctgaccccaattggcacccctctgccttcagccattccctctggctactgctcacaggacggtcagacagggcgacagcctctcccgccctacacccctgccatgatgcacagaagcaatggtcacaccctgacccagcctcccggtccaagaggctgtgagggcgatggcccagagcatggggtagaagagggaacgaggaagagggtctcgctgcctcagtggccacctccttctcgagcaaagtgggcccacgcagccagagaggacagccttcctgaggaatcctcagcccctgattttgcaaacctgaagcactatcaaaaacagcagagtcttccaagtttatgcagcacttctgacccagacacacctcttggggccccgagcactccagggaggatctccctccgaatatctgagtctgtcctgcgggactccccgccacctcatgaggattatgaagacgaagtgtttgtgagggatccgcaccccaaggccacgtccagccccacatttgaacctcttcccccacccccacctcctccaccgagtcaggaaaccccggtgtatakcatggatgacttccctccacctcctccccacactgtatgtgaggcgcagctggacagtgaggatcccgaggggccacgccccagcttcaacaaactttctaaagtgacaattgcaagggaaaggcacatgcctggtgcagcccatgtggtaggtagtcagacactggcttccagactccaaacttctatcaagggttcagaggctgagtccacaccaccctccttcatgagcgttcacgcccaacttgctgggtctcttggtgggcagccagcacccatccagactcaaagcctcagccatgatccagtcagtggaactcagggtttagaaaagaaagtcagtcctgatcctcagaagagttcagaagacatcagaacagaggctttggccaaggaaattgtccaccaagacaaatctctagcagacattttggatccagactccaggctgaagacaacaatggacctgatggaaggtttgtttccccgagatgtgaacttgctgaaggaaaacagtgtaaagaggaaggccatacagagaactgtcagctcttcaggatgtgaaggcaagaggaatgaagacaaggaagcagtgagcatgttggttaactgccctgcctactacagtgtgtctgctcccaaggctgagctactgaacaaaatcaaagagatgccagcagaagtgaatgaggaagaggaacaggcagatgtcaatgaaaagaaggctgagctcattggaagtctcacccacaagctggagaccctccaggaggcgaaggggagcctgctcacggacatcaagctcaacaacgccctgggagaagaggtggaggctctgatcagcgagctctgcaagcccaatgagtttgacaagtataggatgttcataggggatttggacaaggtggtcaacctgctgctctccctctcggggcgtctagcccgtgttgagaatgtccttagcggccttggtgaagatgccagtaatgaagaaaggagctctctttacgagaaaaggaagatcctggctggtcagcatgaggatgcccgggagctgaaggagaacctggatcgcagggagcgagtagtgctgggcatcttggccaattacctttcagaggagcagctccaggactaccagcacttcgtgaaaatgaagtccacgctcctcattgagcaacggaagctggatgacaagatcaagctgggccaggagcaggtcaagtgtctgctggagagcctgccctcagatttcattcccaaggctggggccctggctctgcccccaaacctcacgagtgagcccattcctgctgggggctgtactttcagtggtattttcccaacattaacctctccactttaacctcttctaaaatacccaaccaaaagatcactgtttctctcaacactatttaatctgaaaaatgtttcagtacaaaccactgtttgaactatctgggttattggtgtttgttcctgatgaaaggaaaaaaattctctccaggaggaagcctttttccttcttgcccttcctgattgatcttctgagagctcgaatgctgctggacacgtaccccttctattattactttgtagtagaaagaaagttaatgaaactgagaactgattggagggtgtttgatcatttagtttttaacaggctgaggcaacatggatcagtgtgtgtccccctcaggaatgtatccacagtggccttccttgctggtgggcagtgtatcctgatggcagggtacaagtaccattaatgaagggtctgcaacataaagccttaaaaagacacacactaagaaaactgtaaaaccttgaacattgttatttatattttttaaaatggaaaagatcactatgtttgttgtgctaaccacttatttgattctgttttgtggtggacatagatgattacgtttgagctttgtattttgtgaaaaccttaatgaaatgaattccaaagataaaaaaaaaaaaaaaaa

SEQ ID NO: 5 - protein, 709 aa, Homo sapiens
  UNSURE (255)
MLPLFHHLTPRWGGSGCKAIGDSSVPSECPGTLDHQRQASRTPCPRPPLAGTQGLVTDTRAAPLTPIGTPLPSAIPSGYCSQDGQTGRQPLPPYTPAMMHRSNGHTLTQPPGPRGCEGDGPEHGVEEGTRKRVSLPQWPPPSRAKWAHAAREDSLPEESSAPDFANLKHYQKQQSLPSLCSTSDPDTPLGAPSTPGRISLRISESVLRDSPPPHEDYEDEVFVRDPHPKATSSPTFEPLPPPPPPPPSQETPVYXMDDFPPPPPHTVCEAQLDSEDPEGPRPSFNKLSKVTIARERHMPGAAHVVGSQTLASRLQTSIKGSEAESTPPSFMSVHAQLAGSLGGQPAPIQTQSLSHDPVSGTQGLEKKVSPDPQKSSEDIRTEALAKEIVHQDKSLADILDPDSRLKTTMDLMEGLFPRDVNLLKENSVKRKAIQRTVSSSGCEGKRNEDKEAVSMLVNCPAYYSVSAPKAELLNKIKEMPAEVNEEEEQADVNEKKAELIGSLTHKLETLQEAKGSLLTDIKLNNALGEEVEALISELCKPNEFDKYRMFIGDLDKVVNLLLSLSGRLARVENVLSGLGEDASNEERSSLYEKRKILAGQHEDARELKENLDRRERVVLGILANYLSEEQLQDYQHFVKMKSTLLIEQRKLDDKIKLGQEQVKCLLESLPSDFIPKAGALALPPNLTSEPIPAGGCTFSGIFPTLTSPL

SEQ ID NO: 6 - DNA, 190 bp, Homo sapiens
  unsure (4)
  unsure (30)
  unsure (97)
  unsure (154)
  unsure (174)
cgangcgtctgcacccacacgctcacgaancatcaggagcctgtctatagcgtatctttcagccctgatgggaagtacttggccagtggatccttcnacaagtgcgtccatatctggaatactcagagtggaaatcttgtccacagctaccgangcactggcggcatcttccangtgtgctggaacgccc

SEQ ID NO: 7 - DNA, 391 bp, Homo sapiens
gagtggaaaggcagggggaggggcagggagcaacagaaggaagagacaacaagcccaagacagcttccatttcagacggaaggcccccagaagatagaattccagccgactgaaaaaccacccaatgaacaaagaagattttagaaaatagaaagctgttgggattacaaagttgcgcgtttcatcggtacaaactggtctttgaacctcctttgtgagagcaattgtagtgtccaaattgttagggaaaacaaaaaaaaaaaatcccaaggaggagggtttttcccccttccctgtttggtttatcacagcattttgctttttttttggcacagctttttacgtttctttccattcagccatcacagagcctgttccgggtggaaaccaa

SEQ ID NO: 8 - protein, 62 aa, Homo sapiens
TSFVRAIVVSKLLGKTKKKNPKEEGFSPFPVWFITAFCFFFGTAFYVSFHSAITEPVPGGNQ

SEQ ID NO: 9 - DNA, 267 bp, Homo sapiens
  unsure (21)
tctctctctctctccctctgnacctttctcatagttgcttcagatcttaggtctcaagggcactttggcgcgtagtaagtgctttatgtaagaaggcagggcaggggggctttttacaggagaaaaaaaaatgacttataagagaaagagcctggagtatttttggaaaaaaaaataatatttttatgttaaaacaattttaaaatcttaaaatggccatcagacatagagagctttgtgtgattcatgttttaaaaaaaaaaaaaa

SEQ ID NO: 10 - DNA, 2178 bp, Homo sapiens
cttttaacggtcactcggccttaarccgtcctgggtgtatgtgaggctgaaagggaaggargaacagggtctgggccgctgtgtgctctgccgtcatctctaaraaacaaccataccactgaatgctactgtgtacttacaaaccacactcatattcgtcacgtcatttcatcttcacccccacctccaaatgaggcttgaaatgaagatgaagatgttcctccctcccctttcaaccatggacctcacactgtggacttcctcttagagcctctgagttaggtacccaagccaaggcacaatgtccctgccccccataagactgcccagcccctatggctctgatcggctggtacagctagcagccaggctccggccagcactctgtgatactctgatcaccgtagggagccaggagttccccgcccacagcctggtgctagcaggtgtcagccagcagctgggccgcaggggccagtgggctctgggagaaggcatcagcccttctacctttgcccagctcctgaactttgtgtatggggagagtgtagagctgcagcctggagagctaaggccccttcaggaggcggccagggccttgggagtgcagtccctggaagaggcatgctggagggctcgaggggacagggctaaaaagccagatccaggcctgaagaaacatcaggaggagccagagaaaccctcaaggaatcctgagagagaactgggggaccctggagagaagcagaaaccagaacaggtttctagaactggtgggagagaacaggagatgttgcacaagcactcgccaccaagaggcagacccgagatggcaggagcaacgcaggaggctcagcaggaacagaccaggtcaaaggagaaacgcctccaagcccctgttggccaaaggggagcagatgggaagcatggagtgctcacgtggttgagggaaaatccagggggctctgaggaaagtctgcgcaagctccctggcccccttcccccagcaggctccctgcaaaccagcgtcacccctaggccctcgtgggctgaggccccttggttggtggggggccagcctgccctgtggagcatcctgctgatgccgcccagatatggcattcccttctaccatagcacccccaccactggagcctggcaggaggtctggcgggaacagaggatcccactgtccctaaatgcccccaaagggctctggagccagaaccagttggcctcctccagccctaccccaggttccctcccccagggccccgcacagctcagccctggggagatggaagagtctgatcaggggcacacaggcgcacttgcaacctgtgcgggtcatgaggacaaggcaggctgcccacctcgcccgcaccctcccccggcccctcctgctcggtctcggccctatgcgtgctctgtctgtggaaagaggttttcactcaagcatcagatggagacgcactaccgagtccacacaggagagaagcccttctcctgtagcctttgtcctcagcgctcccgggacttctcggccatgaccaagcacctgcggacacacggggccgctccgtaccgctgctccctgtgcggggccggctgtcccagcctggcctccatgcaggcgcacatgcgcggtcactcgcccagccaactcccgcccggatggaccatccgctccaccttcctctactcctcctcgaggccgtctcggccctcgacctctccctgttgtccttcttcctccaccacctgacggggtgtcggtagcgtcttagccaagagtccaattaaagaacgaaaagcgggccggctcggcttctgacctggcaccgctgctacggcggcctagcaaattccgccccagaagcgccccaggaagggcgccgagtgccctctcctggacgatcgcgggtcgcagaagcccaggccagcgagccctacagagtgaggacactgaagtgtgcaggagcgaaggttaacagtaggggagattgtcgatctcatcaccataataaagagtttcctgttgccctcccttcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - protein, 487 aa, Homo sapiens
MSLPPIRLPSPYGSDRLVQLAARLRPALCDTLITVGSQEFPAHSLVLAGVSQQLGRRGQWALGEGISPSTFAQLLNFVYGESVELQPGELRPLQEAARALGVQSLEEACWRARGDRAKKPDPGLKKHQEEPEKPSRNPERELGDPGEKQKPEQVSRTGGREQEMLHKHSPPRGRPEMAGATQEAQQEQTRSKEKRLQAPVGQRGADGKHGVLTWLRENPGGSEESLRKLPGPLPPAGSLQTSVTPRPSWAEAPWLVGGQPALWSILLMPPRYGIPFYHSTPTTGAWQEVWREQRIPLSLNAPKGLWSQNQLASSSPTPGSLPQGPAQLSPGEMEESDQGHTGALATCAGHEDKAGCPPRPHPPPAPPARSRPYACSVCGKRFSLKHQMETHYRVHTGEKPFSCSLCPQRSRDFSAMTKHLRTHGAAPYRCSLCGAGCPSLASMQAHMRGHSPSQLPPGWTIRSTFLYSSSRPSRPSTSPCCPSSSTT

SEQ ID NO: 12 - DNA, 2436 bp, Homo sapiens
caacaaccatctacagctgtattcttgttcccttgatggcacaattaaactgtgggactatatagatggcatcttaataaagactttcatagttggatgtaaacttcatgccctctttactcttgcccaagctgaggattctgtctttgttatagtgaataaagaaaaaccagatatatttcagctggtttcagtgaaactgccaaaatcctcaagccaggaagtagaagccaaggagctgtcctttgttttggattacataaaccagtcacccaagtgcattgcctttggaaacgagggagtatatgttgctgcagtacgggaattttacttgtctgtttattttttcaaaaagaaaacaacatcaaggtttactttatcatcatcaagaaataagaagcatgctaaaaacaattttacgtgtgtagcatgtcacccaacggaagactgcatcgcatctggtcacatggatggcaaaattcgtctttggaggaatttttatgatgataagaaatatacgtacacatgtttacattggcaccatgatatggttatggatttggctttttcagtgacaggcaccagtctgctgagtggcggtcgtgaatctgtacttgtagagtggcgcgatgcaacagagaagaataaggagtttctcccgcgtttaggagctactattgaacatatctcagtctcgcctgcaggagatttattctgcacttctcactctgataataagataataattattcaccgaaaccttgaagcatccgcagtaattcaaggcctagtgaaagataggagtatcttcactggtttgatgattgatccaagaactaaagctttggttttgaatggaaaacctggccacctgcagttttattctctccagagtgataaacagttatacaatttagatattatacagcaagaatatattaatgattatggtctgatccaaattgaactaacaaaggctgcatttggctgctttggtaactggcttgcaacagtggaacagcggcaagaaaaggaaactgagcttgaattgcaaatgaaactgtggatgtataataagaaaacacaagggtttattcttaacactaaaattaacatgccacacgaagactgcattacagctctctgtttctgtaatgcagaaaaatctgaacagcccaccttggttacagctagcaaagatggttacttcaaagtatggatattaacagatgactctgacatatacaaaaaagctgttggctggacctgtgactttgttggtagttatcacaagtatcaagcaactaactgttgtttctccgaagatggttctttactagcagttagttttgaggaaatagtcacaatatgggattctgtaacatgggaacttaaatgtacattttgccaacgagctgggaaaataaggcacctttgctttgggagattgacgtgttcaaagtatctacttggtgctactgaaaatggcattctttgctgttggaatctgctgagctgtgcattggagtggaatgcaaaattaaatgttagagttatggaacccgatcctaattcagagaatattgctgcaatctctcagtcttcagtgggttcagacttgtttgtatttaaacctagtgagccaaggccattgtatattcaaaagggtatctccagagagaaagtccagtggggagtgtttgttccacgagatgtccctgaatccttcacctcagaagcttaccagtggctaaatagatcccagttttacttcctaacaaaatcacagagtttattgacattcagtacaaagtctccagaagaaaaactcacaccaacaagcaaacagctgctagcagaagaaagtcttcccacaaccccattttatttcatattgggaaaacacaggcaacagcaggatgaaaaactaaacgaaactttagagaatgagctggtacaactacccttaacagaaaacatacccgcaattagtgagcttcttcacactccagcccatgtcctgccatctgctgctttcctgtgctccatgtttgtaaattcattgctgctgtctaaagagactaagagtgctaaggaaattcctgaagatgtagatatggaagaagaaaaagaaagtgaagattcagatgaagaaaatgattttaccgaaaaagtccaggatacaagtaacacaggtttaggagaagacattatacatcagttgtcaaaatctgaagaaaaagaactgagaaaatttaggaaaatagactacagctggatagctgccctttaagccttggagatggggaggatccttggactttgtgtttttgattgtatgttgatattctaaaaacatctattttaatgttatttctgttctaaaaataagataataaatattaacaaactttaaaaaaaaaaaaaaaaaaaaaa

SEQ ID NO: 13 - protein, 763 aa, Homo sapiens
NNHLQLYSCSLDGTIKLWDYIDGILIKTFIVGCKLHALFTLAQAEDSVFVIVNKEKPDIFQLVSVKLPKSSSQEVEAKELSFVLDYINQSPKCIAFGNEGVYVAAVREFYLSVYFFKKKTTSRFTLSSSRNKKHAKNNFTCVACHPTEDCIASGHMDGKIRLWRNFYDDKKYTYTCLHWHHDMVMDLAFSVTGTSLLSGGRESVLVEWRDATEKNKEFLPRLGATIEHISVSPAGDLFCTSHSDNKIIIIHRNLEASAVIQGLVKDRSIFTGLMIDPRTKALVLNGKPGHLQFYSLQSDKQLYNLDIIQQEYINDYGLIQIELTKAAFGCFGNWLATVEQRQEKETELELQMKLWMYNKKTQGFILNTKINMPHEDCITALCFCNAEKSEQPTLVTASKDGYFKVWILTDDSDIYKKAVGWTCDFVGSYHKYQATNCCFSEDGSLLAVSFEEIVTIWDSVTWELKCTFCQRAGKIRHLCFGRLTCSKYLLGATENGILCCWNLLSCALEWNAKLNVRVMEPDPNSENIAAISQSSVGSDLFVFKPSEPRPLYIQKGISREKVQWGVFVPRDVPESFTSEAYQWLNRSQFYFLTKSQSLLTFSTKSPEEKLTPTSKQLLAEESLPTTPFYFILGKHRQQQDEKLNETLENELVQLPLTENIPAISELLHTPAHVLPSAAFLCSMFVNSLLLSKETKSAKEIPEDVDMEEEKESEDSDEENDFTEKVQDTSNTGLGEDIIHQLSKSEEKELRKFRKIDYSWIAAL

SEQ ID NO: 14 - DNA, 137 bp, Homo sapiens
  unsure (2)
  unsure (18)
  unsure (29)..(30)
  unsure (36)
  unsure (44)
  unsure (49)
  unsure (62)
  unsure (67)
  unsure (80)
  unsure (93)
  unsure (99)
  unsure (108)
  unsure (112)
  unsure (121)
gngggggaggatgaatantcggaagctgnngcggcngcgggganccaangaagaggttccgnacccngcttacgaaccgnggactcttgactncccacnccctgaacnctcncctccaaangcgtcatggatggaat

SEQ ID NO: 15 - DNA, 539 bp, Homo sapiens
gtggagggagagtgaattctgggaatctctcagcagctttttgccaaacagatgggccaggagccgcggaaccaggctgaggaatgttgcctcacgatctctcatatccattcctggcacccaccagcccagggaatgcctctaccagttgtcagcgagaggcttacacagcatcttaaataaaagggattattgaaccaagaggccagggactgatggaaatgcccaccttgctggctcattgaaaaagtttggcaaggttgtcaggagacatgaattagatgggcttgggtcttgtgccctttgctaaaccaagtgctgtattgggaaagagacggggagagaagtgttggagatgctctttagtcaggcctgagtcacttgcccaaccctggagttggagttggggatggagccaggatctccaaaccacatgcccctagagtttcagggaaaatatggattgtgaattgaagatggggggtgatgtaaggcagacaaggacagaaaatccctcttccagctgtgatttggctgtg

SEQ ID NO: 16 - protein, 27 aa, Homo sapiens
MDCELKMGGDVRQTRTENPSSSCDLAV

SEQ ID NO: 17 - DNA, 99 bp, Homo sapiens
  unsure (50)
  unsure (55)..(56)
  unsure (62)
  unsure (64)
  unsure (84)
agagaggcagatccccaatgaggggtcaggacgggtcttggctgcacatnctggnntcatantnatcccatggggagcagcacnttatgaaaaaaaaaa

SEQ ID NO: 18 - DNA, 2608 bp, Homo sapiens
attagccactgcacctggctggtgtctccattttacagatgaggctgtcaccccccaatatcacacagccaagtggcagagttgtgttcaaatgctggcagtcaggccatgctctgtctgartccaractctgccactttctarctgtgtgttctcgggcaggttatttaccaactttgaagtccattttcttatccctaaaagagggctgagaagctcacccctacctttgaaggttgtttgaggattaagcaagaccagcatctgaagggcctgaagcatctagggcatcatcagtgcactttatctctggcctctaagaagtgcatctagaagggacccactgtcacagcttcctggtacccttgcctttgcctgtcaggtagcacttcggtttggacctcactttttcaggatttgttggaaacaagatcccttatcttctctaggtcagatttggggtgaacttggctgacagtattccagggcaggcagaggtcaggctggctggtgacctgctgcctccttcccatcggcagctagttctagggccatccacagggatccccagccctccatagtaccagtgagcctcactggcatgtgccagggccaagagggccttcagcgtcgcctccagtccagccctgtcctcgtattcttggggacactgtggctcagccagcacagtgcctagcccacagtcacccagcaggcgtccctcgagtgggccagtggcaggcacagagctgctctccccttgcctggatgcgtgaggaaaggaactcttctccctgcagcccagccctgcaggctctctgctgccagatgacgagtgaagatgaaagtcctgatgtctattgtctgtgtgcacagagctttggagtggcctggccaggaagaggggaagcgtgtgtggggtcagcacaaggggttgccgggatcttggttactggttctgctctgctgctggttccctctgaaaatgtgggtttctcccctccgtgctgagcctcagaaaggggcagggagccccatgccagcacacatggcccctcggctctctggcctctgcagggtcagcccagctccttcacctctctcctgctttcccctcaggcctcagtgaccccacacccccttcaggcccaagcctggatggtaccggatggaacagcttccagtcgtcggatgccactgagccccccagcccctgctctggcccaggcccccagtatggaaagccgacccccagcgcagacatccctgccagcaagcagcggtctggacgacctagacctcctggggaagaccctcctgcagcagtcgctgcccccggaatcccagcaagtgcggtgggagaagcagcagccaaccccccggctcacactccgggacctgcagaataagagcagcagctgcagctcccccagctccagcgccaccagccttctccacaccgtgtccccagagccccccaggcctccgcagcagcccgtaccaaccgagctctcactggccagcatcactgtgcccctggagtccatcaaacccagcaacatcctgcccgtgactgtgtatgaccagcacggcttccgcatcctcttccattttgcccgggacccactgccagggcgctccgacgtgctggtggtggtggtttccatgctgagcaccgccccccagcccatccgcaacatcgtgttccagtcagctgtccccaaggttatgaaggtgaagctgcagccaccctcgggcacggagctgccagcttttaaccccatcgtccacccctcagcaatcacccaggtcctgctgcttgccaacccccagaaggagaaggttcgcctccgctacaagctcaccttcaccatgggtgaccagacctacaacgagatgggggatgtggaccagttccccccacctgaaacctggggtagcctctagaacagaggggctggggagaggaaggggcagagggaccggtcactgtccagcctggagggaggcattggtggccaaggacaccctttgttgcccatggccattcacccccaggcctggtgcttctccccacacccctgtaggcctcaagtgactcttccccctcctgctccggccccgcccctgctgagccaaacccagtaggaggctgggcctgggtttgtgccgctggggtctccatcaccgggacctggagagggaggggctgtgtagccttggaagaacttgggtcatggggaggaagcacagctgttggggaagggccaggacctcaggcccagccccaaccccagctggggtggggtcttccccacctgtctcttatgccttatgggaaggcccagccataactcgggggccatgctcgagctggggaccagcttaggcctcctatgggtgtcttacgtctgtccatccatctgtccgtggtcagaagtggggtcagtgtgtgagtgagagcaggagtatttatgaaaataaaacgtcgtttttcctggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - protein, 236 aa, Homo sapiens
MESRPPAQTSLPASSGLDDLDLLGKTLLQQSLPPESQQVRWEKQQPTPRLTLRDLQNKSSSCSSPSSSATSLLHTVSPEPPRPPQQPVPTELSLASITVPLESIKPSNILPVTVYDQHGFRILFHFARDPLPGRSDVLVVVVSMLSTAPQPIRNIVFQSAVPKVMKVKLQPPSGTELPAFNPIVHPSAITQVLLLANPQKEKVRLRYKLTFTMGDQTYNEMGDVDQFPPPETWGSL

SEQ ID NO: 20 - DNA, 328 bp, Homo sapiens
agacttcgaattcggccttcatggcctaraaaaactgattcacctggcagagacctaccccatccacatgcacagccagctggaccaccttagcctctattactgcaggtgtactctgccagagaatccaaacaatcacaccctccagtactggaaggaccacaacatcgtgacagcagaagtccactgggctaacctgactgtcagtgaatgccaggagatgcatggagagttcatgggatctgcgtgcggccatcatggaccctacactcctgatgtcctcttttggtcctgtattctctttttcaccaccttcatcctctcaagc

SEQ ID NO: 21 - protein, 87 aa, Homo sapiens
MHSQLDHLSLYYCRCTLPENPNNHTLQYWKDHNIVTAEVHWANLTVSECQEMHGEFMGSACGHHGPYTPDVLFWSCILFFTTFILSS

SEQ ID NO: 22 - DNA, 326 bp, Homo sapiens
  unsure (1)
  unsure (31)
  unsure (82)
  unsure (84)
  unsure (129)
naaatgtatttatatgtatgtcttattatanacaaggcagatttcccctggaataaaagtctagaatgtactgcttaatttnanacatgtgtgcaggcaatattatctgtgagtgaaaagtggaataanacgtggattgggtcaactgattatcagcttgttaggagtcctctgtgtgagacatggtggtataattgtgaagttctcactgtatgtggatgttcatgtgaaagatagtactttcttcccgtaaatatcttttgatttccatttgtatggaatcccaatgaatgtatctttggaaaacaaaaaaaaaaaaaaaaaaa

SEQ ID NO: 23 - DNA, 194 bp, Homo sapiens
  unsure (55)
  unsure (73)
  unsure (119)
  unsure (126)
  unsure (137)
  unsure (174)
aagtctcattctcaagagtgatgctgcaaaaccccttttgggccatgatgctgtnatccaggctttagcacanaaaggtctttatgtcactgaccaggaaaaattggtaactgaacganatctccncaagaaacccntacagatgagtgcacatttgggccatgatcgataccncatgatggcttatactgtat

SEQ ID NO: 24 - DNA, 396 bp, Homo sapiens
  unsure (139)
ttcatttttactgtcctgatgttgtcagattagaggatatttgtttgaaagaaactatgtctttggctgatagcctgtataatctgcagctgattcaagaattttgccaagaatacttgaaccagtgttgccatttcantctggaagatatgctctatgctgcttcatccataaagagtaattatttggtgttcatggcggaactgttctggtggtttgaagtggtgaagccgtcttttgtacagcctcgtgttgttcgtccacaaggagctgaacctgtaaaagatatgccttcaattcctgtcttgaatgctgccaaaagaaatgtcttagatagtagttctgacttcccttcaagtggggaaggagctacatttacacagtctcatctcgagg

SEQ ID NO: 25 - protein, 113 aa, Homo sapiens
  UNSURE (28)
MSLADSLYNLQLIQEFCQEYLNQCCHFXLEDMLYAASSIKSNYLVFMAELFWWFEVVKPSFVQPRVVRPQGAEPVKDMPSIPVLNAAKRNVLDSSSDFPSSGEGATFTQSHLE

SEQ ID NO: 26 - DNA, 336 bp, Homo sapiens
  unsure (87)
  unsure (123)
aatttgatgatgaccagaaagtatgctgtggttcttttttaaggatgatcaaaaagcagaaaatgatatggcaatgaaacgggcagntttgttggagaaaagattaagaagggaaaaggaaantcagctccggaaacaacagttggaagcagaaatggagcataagaaggaggaaacaaggcgtaaaactgaggaagaacgtcagaagaaagaagatgagagagcacgcagagaatttattaggcaagaatatatgaggcggaaacaactgaaactaatggaagatatggatacagtaattaaaccccgtcctcaagtagtaaaaaaaaaaaaaaa

SEQ ID NO: 27 - DNA, 917 bp, Homo sapiens
cacctggattatctcagtagtttcccaactggtttccttgtttccattcttgcctccttctgtctactctcaatataacagctagaacaatccttttacaatggaattcagatcatgtttacccctctgttcaaattctccagtgactttccagtttttacatgatctggctcctactacctgtctcactgtgtttcctactactctcctgccctttctcctcttaataaacactgggctcatggtgtttcctttaacatgccaggcatgcttgaccctgtcctgtctcagggccctgctgttccctctgcctggaacattcttcccatagtgtctgcatggctcgctctctcactgctttggattgctgctcaaaagtcaccttatcaaaggcctttcccaaaggtttaaaaatcattctactataaagacacatgcatacatatgtttattgcagcactattcacaataacaaagacttggaaccaacccaaatgcccatcaatgatagactggataaagaaaatatggcacgtaagcaccatggaatactatgcagccataaaaaagaatgagttcatgtcctttgcagggacatggatgaagctggaaaccattattctcagcaaactaacacaggaacagaaaaccaaacaccgcatgttctcactcataagtgggagttgaacaatgagaacatacgggcacagtggggggaacatcacacaccagggcctgtcggggggtgagaggcaagggaagtgatagcattaagagaaatacctaatgtagattatgggttgatgggggcagcaaaccaccatggcacatgtgtacctatgtaacaaacctgcacattctgcacatatatcccagaacttaaagtataattaaagaaaaagaaaaaaaaaaaaaaa

SEQ ID NO: 28 - protein, 76 aa, Homo sapiens
MEFRSCLPLCSNSPVTFQFLHDLAPTTCLTVFPTTLLPFLLLINTGLMVFPLTCQACLTLSCLRALLFPLPGTFFP

SEQ ID NO: 29 - DNA, 351 bp, Homo sapiens
ggctttgaccgctatcgccaggagtggatggactatggctgtgcacaggaggcagagggcaggatgtgcgaggacttccaggatgaggaccacgactcagcctcccctgacacttccttcagcccctatgatggagacctcaccamtacctcctcctccctcttcatcgacagcctcaccacagaagatgacaccaagttgaatccctatgcaggaggagacggccttcagaacaacctgtcccccaagacaaagggcactcctgtgcacctgggcaccatcgtgggcatcgtgctggcagtcctcctcgtggcggccatcatcctggctggaatttacatcaatggccac

SEQ ID NO: 30 - protein, 108 aa, Homo sapiens
  UNSURE (40)
MDYGCAQEAEGRMCEDFQDEDHDSASPDTSFSPYDGDLTXTSSSLFIDSLTTEDDTKLNPYAGGDGLQNNLSPKTKGTPVHLGTIVGIVLAVLLVAAIILAGIYINGH

SEQ ID NO: 31 - DNA, 179 bp, Homo sapiens
  unsure (24)
  unsure (33)
  unsure (56)
  unsure (82)
  unsure (93)
  unsure (99)
  unsure (117)
  unsure (137)
gttgtcagccccttcctttccccngacctaaanataaaagacaaggcaaagcccgnataattttaagacggttttttagganattagtccacnattttnttggtttgatggttttcngaaataaagtccctttcctngctccaaaaaaaaaaaaaaaaaaaaaaaaaaaaaaaaaaaaa

SEQ ID NO: 32 - DNA, 3906 bp, Homo sapiens
ggaaactcagccacctgtgacaaatttgagtgtctctgttgaaaacctctgcacagtaatatggacatggaatccacccgagggagccagctcaaattgtagtctatggtattttagtcattttggcgacaaacaagataagaaaatagctccggaaactcgtcgttcaatagaagtacccctgaatgagaggatttgtctgcaagtggggtcccagtgtagcaccaatgagagtgagaagcctagcattttggttgaaaaatgcatctcacccccagaaggtgatcctgagtctgctgtgactgagcttcaatgcatttggcacaacctgagctacatgaagtgttcttggctccctggaaggaataccagtcccgacactaactatactctctactattggcacagaagcctggaaaaaattcatcaatgtgaaaacatctttagagaaggccaatacttgggttgttcctttgatctgaccaaagtgaaggattccagttttgaacaacacagtgtccaaataatggtcaaggataatgcaggaaaaattaaaccatccttcaatatagtgcctttaacttcccgtgtgaaacctgatcctccacatattaaaaacctctccttccacaatgatgacctatatgtgcaatgggagaatccacagaattttattagcagatgcctattttatgaagtagaagtcaataacagccaaactgagacacataatgttttctacgtccaagaggctaaatgtgagaatccagaatttgagagaaatgtggagaatacatcttgtttcatggtccctggtgttcttcctgatactttgaacacagtcagaataagagtcaaaacaaataagttatgctatgaggatgacaaactctggagtaattggagccaagaaatgagtataggtaagaagcgcaattccacactctacataaccatgttactcattgttccagtcatcgtcgcaggtgcaatcatagtactcctgctttacctaaaaaggctcaagattattatattccctccaattcctgatcctggcaagatttttaaagaaatgtttggagaccagaatgatgatactctgcactggaagaagtacgacatctatgagaagcaaaccaaggaggaaaccgactctgtagtgctgatagaaaacctgaagaaagcctctcagtgatggagataatttatttttaccttcactgtgaccttgagaagattcttcccattctccatttgttatctgggaacttattaaatggaaactgaaactactgcaccatttaaaaacaggcagctcataagagccacaggtctttatgttgagtcgcgcaccgaaaaactaaaaataatgggcgctttggagaagagtgtggagtcattctcattgaattataaaagccagcaggcttcaaactaggggacaaagcaaaaagtgatgatagtggtggagttaatcttatcaagagttgtgacaacttcctgagggatctatacttgctttgtgttctttgtgtcaacatgaacaaattttatttgtaggggaactcatttggggtgcaaatgctaatgtcaaacttgagtcacaaagaacatgtagaaaacaaaatggataaaatctgatatgtattgtttgggatcctattgaaccatgtttgtggctattaaaactcttttaacagtctgggctgggtccggtggctcacgcctgtaatcccagcaatttgggagtccgaggcgggcggatcactcgaggtcaggagttccagaccagcctgaccaaaatggtgaaacctcctctctactaaaactacaaaaattaactgggtgtggtggcgcgtgcctgtaatcccagctactcgggaagctgaggcaggtgaattgtttgaacctgggaggtggaggttgcagtgagcagagatcacaccactgcactctagcctgggtgacagagcaagactctgtctaaaaaacaaaacaaaacaaaacaaaacaaaaaaacctcttaatattctggagtcatcattcccttcgacagcattttcctctgctttgaaagccccagaaatcagtgttggccatgatgacaactacagaaaaaccagaggcagcttctttgccaagacctttcaaagccattttaggctgttaggggcagtggaggtagaatgactccttgggtattagagtttcaaccatgaagtctctaacaatgtattttcttcacctctgctactcaagtagcatttactgtgtctttggtttgtgctaggcccccgggtgtgaagcacagaccccttccaggggtttacagtctatttgagactcctcagttcttgccactttttttttaatctccaccagtcatttttcagaccttttaactcctcaattccaacactgatttccccttttgcattctccctccttcccttccttgtagccttttgactttcattggaaattaggatgtaaatctgctcaggagacctggaggagcagaggataattagcatctcaggttaagtgtgagtaatctgagaaacaatgactaattcttgcatattttgtaacttccatgtgagggttttcagcattgatatttgtgcattttctaaacagagatgaggtggtatcttcacgtagaacattggtattcgcttgagaaaaaaagaatagttgaacctatttctctttctttacaagatgggtccaggattcctcttttctctgccataaatgattaattaaatagcttttgtgtcttacattggtagccagccagccaaggctctgtttatgcttttggggggcatatattgggttccattctcacctatccacacaacatatccgtatatatcccctctactcttacttcccccaaatttaaagaagtatgggaaatgagaggcatttcccccaccccatttctctcctcacacacagactcatattactggtaggaacttgagaactttatttccaagttgttcaaacatttaccaatcatattaatacaatgatgctatttgcaattcctgctcctaggggaggggagataagaaaccctcactctctacaggtttgggtacaagtggcaacctgcttccatggccgtgtagaagcatggtgccctggcttctctgaggaagctggggttcatgacaatggcagatgtaaagttattcttgaagtcagattgaggctgggagacagccgtagtagatgttctactttgttctgctgttctctagaaagaatatttggttttcctgtataggaatgagattaattcctttccaggtattttataattctgggaagcaaaacccatgcctccccctagccatttttactgttatcctatttagatggccatgaagaggatgctgtgaaattcccaacaaacattgatgctgacagtcatgcagtctgggagtggggaagtgatcttttgttcccatcctcttcttttagcagtaaaatagctgagggaaaagggagggaaaaggaagttatgggaatacctgtggtggttgtgatccctaggtcttgggagctcttggaggtgtctgtatcagtggatttcccatcccctgtgggaaattagtaggctcatttactgttttaggtctagcctatgtggattttttcctaacatacytaagcaaacccagtgtcaggatggtaattcttattctttcgttcagttaagtttttcccttcatctgggcactgaagggatatgtgaaacaatgttaacatttttggtagtcttcaaccagggattgtttctgtttaacttcttataggaaagcttgagtaaaataaatattgtctttttgtatgtcacccaaaaaaaaaaaaaaaaaaaaaaaaaaa

SEQ ID NO: 33 - protein, 286 aa, Homo sapiens
MKCSWLPGRNTSPDTNYTLYYWHRSLEKIHQCENIFREGQYLGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 34 - DNA, 1605 bp, Homo sapiens
acgctgaccatcacaggcacacagaggcacatccacctcacatccacctcatacttgtgtactctcagggttcagtctttcatcctatccctctctgatctgtgcctcccaataccttccaagatgtttacagagacccttctccctgtgcagttaggagtgtaaggcaagagagcccctacttcatggggcagatcaagagctgagaccaaagatggtctatgttgctgaccttgtcctgtcctcctgctgtcttaaactatgatccctgctgcggtcactgaagcctttccctgtgagcagtggtgtgtgagagccaggcgtccctctgcctgcccactcagtggcaacacccgggagctgttttgtcctttgtggagcctcagcagttccctctttcagaactcactgccaagagccctgaacaggagccaccatgcagtgcttcagcttcattaagaccatgatgatcctcttcaatttgctcatctttctgtgtggtgcagccctgttggcagtgggcatctgggtgtcaatcgatggggcatcctttctgaagatcttcgggccactgtcgtccagtgccatgcagtttgtcaacgtgggctacttcctcatcgcagccggcgttgtggtctttgctcttggtttcctgggctgctatggtgctaagactgagagcaagtgtgccctcgtgacgttcttcttcatcctcctcctcatcttcattgctgaggttgcagctgctgtggtcgccttggtgtacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcgagctggctgccatgattgtgtccatgtatctgtactgcaatctacaataagtccacttctgcctctgccactactgctgccacatgggaactgtgaagaggcaccctggcaagcagcagtgattgggggaggggacaggatctaacaatgtcacttgggccagaatggacctgccctttctgctccagacttgggggtagatagggaccactccttttaggcgatgcctgactttccttccattggtgggtggatgggtggggggcattccagagcctctaaggtagccagttctgttgcccattcccccagtctattaaacccttgatatgccccctaggcctagtggtgatcccagtgctctactgggggatgagagaaaggcattttatagcctgggcataagtgaaatcagcagagcctctgggtggatgtgtagaaggcacttcaaaatgcataaacctgttacaatgttgaaaaaaaaaaaaaaaaa

SEQ ID NO: 35 - protein, 241 aa, Homo sapiens
MQCFSFIKTMMILFNLLIFLCGAALLAVGIWVSIDGASFLKIFGPLSSSAMQFVNVGYFLIAAGVVVFALGFLGCYGAKTESKCALVTFFFILLLIFIAEVAAAVVALVYTTMAEHFLTLLVVPAIKKDYGSQEDFTQVWNTTMKGLKCCGFTNYTDFEDSPYFKENSAFPPFCCNDNVTNTANETCTKQKAHDQKVEGCFNQLLYDIRTNAVTVGGVAAGIGGLELAAMIVSMYLYCNLQ

SEQ ID NO: 36 - DNA, 377 bp, Homo sapiens
  unsure (346)
caacctcgtggtcaccgcaccggggctgatcaagggtgacgcctgcttcacatctctaatgaacaccctcatgacgtcgctaccagcactagtgcagcaacagggaaggctgcttctggctgctaatgtggccaccctggggctcctcatggcccggctccttagcacctctccagctcttcagggaacaccagcatcccgagggttcttcgcagctgccatcctcttcctatcacagtcccacgtggcgcgggccaccccgggctcagaccaggcagtgctagccctgtcccctgagtatgagggcatctgggccgacctgcaggagctctggttcctgggcatncaagccttcaccggctgtgtgcctctgctgc

SEQ ID NO: 37 - protein, 106 aa, Homo sapiens
  UNSURE (96)
MNTLMTSLPALVQQQGRLLLAANVATLGLLMARLLSTSPALQGTPASRGFFAAAILFLSQSHVARATPGSDQAVLALSPEYEGIWADLQELWFLGXQAFTGCVPLL

SEQ ID NO: 38 - DNA, 245 bp, Homo sapiens
  unsure (3)
  unsure (17)
  unsure (34)
  unsure (46)
  unsure (65)
  unsure (71)
  unsure (142)
  unsure (214)
tangactcctctctgcngagacgcgactggcggntccagcagggantaccttttttataaacccngggggnccacacacacacacacacacacacacacacacacacacacacacacacacatttttgatcccttgcttccntcccccagtgcgttctgtgatcgccaagttcaaagctgtgcacatgtggacactcaataaatgttcattggngacaaaaaaaaaaaaaaaaaaaaaaaaaaaa

SEQ ID NO: 39 - DNA, 2384 bp, Homo sapiens
ccaaaacatgctcaaaagtagaacattttgtttcaatattaggaaagtgctttgaatccccttggacgacaaaagcgttgtctgagacagcatgcgaagactcagaggaaaacaagcagagaataacaggtgcccagactctaccaaagcatgtttctaccagcagtgatgaagggagccccagtgccagtacaccaatgatcaataaaactggctttaaattttcagctgagaagcctgtgattgaagttcccagcatgacaatcctggataaaaaggatggagagcaggccaaagccctgtttgagaaagtgaggaagttccgtgcccatgtggaagatagtgacttgatctataaactctatgtggtccaaacagttatcaaaacagccaagttcatttttattctctgctatacagcgaactttgtcaacgcaatcagctttgaacacgtctgcaagcccaaagttgagcatctgattggttatgaggtatttgagtgcacccacaatatggcttacatgttgaaaaagcttctcatcagttacatatccattatttgtgtttatggctttatctgcctctacactctcttctggttattcaggatacctttgaaggaatattctttcgaaaaagtcagagaagagagcagttttagtgacattccagatgtcaaaaacgattttgcgttccttcttcacatggtagaccagtatgaccagctatattccaagcgttttggtgtgttcttgtcagaagttagtgaaaataaacttagggaaattagtttgaaccatgagtggacatttgaaaaactcaggcagcacatttcacgcaacgcccaggacaagcaggagttgcatctgttcatgctgtcgggggtgcccgatgctgtctttgacctcacagacctggatgtgctaaagcttgaactaattccagaagctaaaattcctgctaagatttctcaaatgactaacctccaagagctccacctctgccactgccctgcaaaagttgaacagactgcttttagctttcttcgcgatcacttgagatgccttcacgtgaagttcactgatgtggctgaaattcctgcctgggtgtatttgctcaaaaaccttcgagagttgtacttaataggcaatttgaactctgaaaacaataagatgataggacttgaatctctccgagagttgcggcaccttaagattctccacgtgaagagcaatttgaccaaagttccctccaacattacagatgtggctccacatcttacaaagttagtcattcataatgacggcactaaactcttggtactgaacagccttaagaaaatgatgaatgtcgctgagctggaactccagaactgtgagctagagagaatcccacatgctattttcagcctctctaatttacaggaactggatttaaagtccaataacattcgcacaattgaggaaatcatcagtttccagcatttaaaacgactgacttgtttaaaattatggcataacaaaattgttactattcctccctctattacccatgtcaaaaacttggagtcactttatttctctaacaacaagctcgaatccttaccagtggcagtatttagtttacagaaactcagatgcttagatgtgagctacaacaacatttcaatgattccaatagaaataggattgcttcagaacctgcagcatttgcatatcactgggaacaaagtggacattctgccaaaacaattgtttaaatgcataaagttgaggactttgaatctgggacagaactgcatcacctcactcccagagaaagttggtcagctctcccagctcactcagctggagctgaaggggaactgcttggaccgcctgccagcccagctgggccagtgtcggatgctcaagaaaagcgggcttgttgtggaagatcacctttttgataccctgccactcgaagtcaaagaggcattgaatcaagacataaatattccctttgcaaatgggatttamactaagataatatatgcacagtgatgtgcaggaacaacttcctagattgcaagtgctcacgtacaagttattacaagataatgcattttaggagtagatacatcttttaaaataaaacagagaggatgcatagaaggctgatagaagacataactgaatgttcaatgtttgtagggttttaagtcattcatttccaaatcatttttttttttcttttggggaaagggaaggaaaaattataatcactaatcttggttctttttaaattgtttgtaacttggatgctgccgctactgaatgtttacaaattgcttgcctgctaaagtaaatgattaaattgacattttcttactataaaaaaaaaaa

SEQ ID NO: 40 - protein, 614 aa, Homo sapiens
  UNSURE (607)
MINKTGFKFSAEKPVIEVPSMTILDKKDGEQAKALFEKVRKFRAHVEDSDLIYKLYVVQTVIKTAKFIFILCYTANFVNAISFEHVCKPKVEHLIGYEVFECTHNMAYMLKKLLISYISIICVYGFICLYTLFWLFRIPLKEYSFEKVREESSFSDIPDVKNDFAFLLHMVDQYDQLYSKRFGVFLSEVSENKLREISLNHEWTFEKLRQHISRNAQDKQELHLFMLSGVPDAVFDLTDLDVLKLELIPEAKIPAKISQMTNLQELHLCHCPAKVEQTAFSFLRDHLRCLHVKFTDVAEIPAWVYLLKNLRELYLIGNLNSENNKMIGLESLRELRHLKILHVKSNLTKVPSNITDVAPHLTKLVIHNDGTKLLVLNSLKKMMNVAELELQNCELERIPHAIFSLSNLQELDLKSNNIRTIEEIISFQHLKRLTCLKLWHNKIVTIPPSITHVKNLESLYFSNNKLESLPVAVFSLQKLRCLDVSYNNISMIPIEIGLLQNLQHLHITGNKVDILPKQLFKCIKLRTLNLGQNCITSLPEKVGQLSQLTQLELKGNCLDRLPAQLGQCRMLKKSGLVVEDHLFDTLPLEVKEALNQDINIPFANGIXTKIIYAQ

SEQ ID NO: 41 - DNA, 2386 bp, Homo sapiens
ccaaaacatcaaacccttttcttgtagcagcacaggattctgagacagattatgtcacaacagataatttaacaaaggtgactgaggaagtcgtggcaaacatgcctgaaggcctgactccagatttagtacaggaagcatgtgaaagtgaattgaatgaagttactggtacaaagattgcttatgaaacaaaaatggacttggttcaaacatcagaagttatgcaagagtcactctatcctgcagcacagctttgcccatcatttgaagagtcagaagctactccttcaccagttttgcctgacattgttatggaagcaccattgaattctgcagttcctagtgctggtgcttccgtgatacagcccagctcatcaccattagaagcttcttcagttaattatgaaagcataaaacatgagcctgaaaaccccccaccatatgaagaggccatgagtgtatcactaaaaaaagtatcaggaataaaggaagaaattaaagagcctgaaaatattaatgcagctcttcaagaaacagaagctccttatatatctattgcatgtgatttaattaaagaaacaaagctttctgctgaaccagctccggatttctctgattattcagaaatggcaaaagttgaacagccagtgcctgatcattctgagctagttgaagattcctcacctgattctgaaccagttgacttatttagtgatgattcaatacctgacgttccacaaaaacaagatgaaactgtgatgcttgtgaaagaaagtctcactgagacttcatttgagtcaatgatagaatatgaaaataaggaaaaactcagtgctttgccacctgagggaggaaagccatatttggaatcttttaagctcagtttagataacacaaaagataccctgttacctgatgaagtttcaacattgagcaaaaaggagaaaattcctttgcagatggaggagctcagtactgcagtttattcaaatgatgacttatttatttctaaggaagcacagataagagaaactgaaacgttttcagattcatctccaattgaaattatagatgagttccctacattgatcagttctaaaactgattcattttctaaattagccagggaatatactgacctagaagtatcccacaaaagtgaaattgctaatgccccggatggagctgggtcattgccttgcacagaattgccccatgacctttctttgaagaacatacaacccaaagttgaagagaaaatcagtttctcagatgacttttctaaaaatgggtctgctacatcaaaggtgctcttattgcctccagatgtttctgctttggccactcaagcagagatagagagcatagttaaacccaaagttcttgtgaaagaagctgagaaaaaacttccttccgatacagaaaaagaggacagatcaccatctgctatattttcagcagagctgagtaaaacttcagttgttgacctcctgtactggagagacattaagaagactggagtggtgtttggtgccagcctattcctgctgctttcattgacagtattcagcattgtgagcgtaacagcctacattgccttggccctgctctctgtgaccatcagctttaggatatacaagggtgtgatccaagctatccagaaatcagatgaaggccacccattcagggaagttgctatatctgaggagttggttcagaagtacagtaattctgctcttggtcatgtgaactgcacgataaaggaactcaggcgcctcttcttagttgatgatttagttgattctctgaagtttgcagtgttgatgtgggtatttacctatgttggtgccttgtttaatggtctgacactactgattttggctctcatttcactcttcagtgttcctgttatttatgaacggcatcaggcacagatagatcattatctaggacttgcaaataagaatgttaaagatgctatggctaaaatccaagcaaaaatccctggattgaagcgcaaagctgaatgaaaacgcccaaaataattagtaggagttcatctttaaaggggatattcatttgattatacgggggagggtcagggaagaacgaaccttgacgttgcagtgcagtttcacagatcgttgttagatctttatttttagccatgcactgttgtgaggaaaaattacctgtcttgactgccatgtgttcatcatcttaagtattgtaagctgctatgtatggatttaaaccgtaatcatatctttttcctatctgaggcactggtggaataaaaaacctgtatattttactttgttgcagatagtcttgccgcatcttggcaagttgcagagatggtggagctagaaaaaaaaaaaaaaaa

SEQ ID NO: 42 - protein, 642 aa, Homo sapiens
MPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFREVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 43 - DNA, 344 bp, Homo sapiens
  unsure (13)
  unsure (39)
  unsure (185)
  unsure (260)
ggcggctgcgganccggcggtccttgcgctccccaacancggcgccgggggcgcgggggcgccgtcgggcacagtcccggtgctcttctgtttctcagtcttcgcgcgaccctcgtcggtgccacacggggcgggctacaagctgctcatccagaagttcctcagcctgtacggcgaccagatcnacatgcaccgcaaattcgtggtgcagctgttcgccgaggagtggggccagtacgtggacttgcccaagggcttcncggtgagcgagcgctgcaaggtgcgcctcgtgccgctgcatatccagctcactaccctgggaaatcttacaccttcaagcactg

SEQ ID NO: 44 - DNA, 631 bp, Homo sapiens
  unsure (73)
  unsure (369)
acatgattcagagtcctgtgggtaaattcatatgcaataatcttattccaatcaatctgtaaagtaaaagcantacatccacattaacattataacatcttacagtaatataaaagccaaatcattgttggtacgtcattttctttaaagtgaacaatttaagaaaacttcacaagagtctgcactttggaaagatacgatcagagtacacagtagagacaaaacaggcatcttcattgtaattttttttaataaataaaagcacattaacaaaaaaggaaggtaagcagcaccggaagcctttgacgtttgtaactaaatgctggtactcaattgaatcgagctggttaagtttcactaggaggcgcnaaaaaggagccgtttttgacttaacattttaattctagtagagataagaagagcttgtgtgggcttacagtccttcacctgactgtccttcaccagtgagtagcataccagttcttcaaatgtcctatactttggaaagcagacccgactctggagcactcgccttaattagattctgaatttccttgaattttggatggtccttatcagctaccagctgaagcagaacagcctcactcgtggtcactatgatcccggttcg

SEQ ID NO: 45 - protein, 22 aa, Homo sapiens
MVLISYQLKQNSLTRGHYDPGS

SEQ ID NO: 46 - DNA, 70 bp, Homo sapiens
aaaaaaaaaaaaaaaaaaaaaaaaaaaaaaaaaaaaaaaaaaaaaaaaaaaaaaaaaaaaaaaaaaaaaa

SEQ ID NO: 47 - DNA, 428 bp, Homo sapiens
agcacgcggtcctgcccgtggacggggcaacgctggcagatgtgatgcgccagcggggcatcaacatgcgctacctgggcaaggtgctggagctggtgctgcggarcccggcccgccaccagctggaccacgtctttaaaatcggcattggagaactcatcacccgctcgsccaagcacatcttcaagacgtacttacagggagtcgagctctccggcctctcagccgccatcagccacttcctgaactgcttcctgagctcctacccaaaccccgtggcccacctgcccgccgacgagctggtctccaagaagcggaataagaggaggaaaaaccggcccccgggggctgcagataacacagcctgggctgtcatgaccccccaggagctctggaagaacatctgccaggaggccaagaactacttt

SEQ ID NO: 48 - protein, 128 aa, Homo sapiens
  UNSURE (21)
  UNSURE (43)
MRQRGINMRYLGKVLELVLRXPARHQLDHVFKIGIGELITRSXKHIFKTYLQGVELSGLSAAISHFLNCFLSSYPNPVAHLPADELVSKKRNKRRKNRPPGAADNTAWAVMTPQELWKNICQEAKNYF

SEQ ID NO: 49 - DNA, 245 bp, Homo sapiens
  unsure (46)
  unsure (138)
  unsure (147)
tggtgggggaggatgtgcccaccctgagacccggaggagacgggcntttgcctgggtttgcggagagccgcttatgggtgtggtccgtccagacaccttgtttcaagggggatgggcgtgagcgggcaagcagagcanccccaccgntgagcaagaacttttttttgtttttaaaccatcacgtcctcatttcacattggaataaagtgagtttttgaaaaaaaaaaaaaaaaaaaaaaaaaaaa

SEQ ID NO: 50 - DNA, 566 bp, Homo sapiens
cagtgagccctttgaaaaataaacatccagatgaagatgctgtggaagctgaggggcatgaggtaaaaagactcaggtttgacaaagaaggtgaagtcagagaaacagccagtcaaacgacttccagcgaaatttcttcagttatggtaggagaaacagaagcatcatcttcatctcaggataaagacaaagatagccgttgtwcccggcagcactgtwcagaagaggatgaagaagaggatgaagaggaagaagaagagtcttttatgacatcaagagaaatgatcccagaaagaaaaaatcaagaaaaagaatctgatgatgccttaactgtgaatgaagagacttctgaggaaaataatcaaatggaggaatctgatgtgtctcaagctgagaaagatttgctacattctgaaggtagtgaaaacgaaggccctgtaagtagtagttcttctgactgccgtgaaacagaagaattagtaggatccaattccagtaaaactggagagattctttcagaatcatccatggaaaatgatgacgaagccacagaagtcaccgatgaa

SEQ ID NO: 51 - protein, 141 aa, Homo sapiens
  UNSURE (21)
  UNSURE (26)
MVGETEASSSSQDKDKDSRCXRQHCXEEDEEEDEEEEEESFMTSREMIPERKNQEKESDDALTVNEETSEENNQMEESDVSQAEKDLLHSEGSENEGPVSSSSSDCRETEELVGSNSSKTGEILSESSMENDDEATEVTDE

SEQ ID NO: 52 - DNA, 531 bp, Homo sapiens
tcatcatggctataaataccaaaacgatttggatccatttatgtttgtaggataatatactactgactgacttgactgtcaggttcacaacagctagatgatatatttatgactatgtctaatagttgaaataaaatctgaatattgatttactatacccaagaggggagaaaaattaaccattgtaaatttttaaaaattttttcaaaaatgttaaaatgaggcaaatttaagtttacaaattttgaaattttcttttgaatatttatgaaattgtcagtaaacttacctaagatcctgtgaccttttgatattttttattttaattgtagtgccatggaccatttgtaaacaaattgatttacttttgttggttgtaagttgaagatttagcattatgactttgaggtctgtggttttatttgtaaacttgcaattgctatatttgcaagggcaaatgtatttctttattaaataaagtacaataatggtgaatgtaccaaaatgacatcacttaaaaaaaaaaaaaaa

SEQ ID NO: 53 - DNA, 1163 bp, Homo sapiens
kctggaaccacgcggargaaggaagagacgcaggcaggctgcggttacccaagcggscacccgggcctcagggacccttccccgagagacggcaccatgacccagggaaagctctccgtggctaacaagcccctgggaccgaggggcagcagcakgtgcatggcgagaagaaggagctccagcagtgccctcagccccaccctcctatgaggaaccacctctggggaggggatgaaggcaggggccttccccccagcccccacagcggtgcctctccaccctagctgggcctatgtggaccccagcagcagctccagctatgacaacggtttccccaccggagaccatgagctcttcaccactttcagctgggatgaccagaaagttcgtcgagtctttgtcagaaaggtctacaccatcctgctgattcagctgctggtgaccttggctgtcgtggctctctttactttctgtgaccctgtcaaggactatgtccaggccaacccaggctggtactgggcatcctatgctgtgttctttgcaacctacctgaccctggcttgctgttctggacccaggaggcatttcccctggaacctgattctcctgaccgtctttaccctgtccatggcctacctcactgggatgctgtccagctactacaacaccacctccgtgctgctgtgcctgggcatcacggcccttgtctgcctctcagtcaccgtcttcagcttccagaccaagttcgacttcacctcctgccagggcgtgctcttcgtgcttctcatgactcttttcttcagcggactcatcctggccatcctcctacccttccaatatgtgccctggctccatgcagtttatgcagcactgggagcgggtgtatttacattgttcctggcacttgacacccagttgctgatgggtaaccgacgccactcgctgagccctgaggagtatatttttggagccctcaacatttacctagacatcatctatatcttcaccttcttcctgcagctttttggcactaaccgagaatgaggagccctccctgccccaccgtcctccagagaatgcgcccctcctggttccctgtccctcccctgcgctcctgcgagaccagatataaaactagctgccaacccaaaaaaaaaaaaaaa

SEQ ID NO: 54 - protein, 270 aa, Homo sapiens
MKAGAFPPAPTAVPLHPSWAYVDPSSSSSYDNGFPTGDHELFTTFSWDDQKVRRVFVRKVYTILLIQLLVTLAVVALFTFCDPVKDYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQYVPWLHAVYAALGAGVFTLFLALDTQLLMGNRRHSLSPEEYIFGALNIYLDIIYIFTFFLQLFGTNRE

SEQ ID NO: 55 - DNA, 624 bp, Homo sapiens
  unsure (123)
cgcaccccctccggccgcgggcgcakcgggggcgctggtggakctgmgaagggccasgtccggcgggcggggcggcggctggcactggctccggactctgcccggccagggcggcggmtccanccgggagggcgacgtggagcggccacktggakcggcccgggggargctggcggcgggakgcgaggcgcgggcggcgcakcakccakgagcgcccacggagstggacccccagakccgcgcggcgccgcagcagttccaggaaggatgttacctttgacgatgacagtgttaatcctgctgctgctccccacgggtcaggctgccccaaaggatggagtcacaaggccagaatctgaagtgcagcatcagctcctgcccaaccccttccagccaggccaggagcagctcggacttctgcagagctacctaaagggactaggaaggacagaagtgcaactggagcatctgagccgggagcaggttctcctctacctctttgccctccatgactatgaccagagtggacagctggatggcctggagctgctgtccatgttgacagctgctctggcccctggagctgccaactctcctaccaccaacccggtgatcttgatagtg

SEQ ID NO: 56 - protein, 119 aa, Homo sapiens
MLPLTMTVLILLLLPTGQAAPKDGVTRPESEVQHQLLPNPFQPGQEQLGLLQSYLKGLGRTEVQLEHLSREQVLLYLFALHDYDQSGQLDGLELLSMLTAALAPGAANSPTTNPVILIV

SEQ ID NO: 57 - DNA, 80 bp, Homo sapiens
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - DNA, 2160 bp, Homo sapiens
agacagggaatactttattcaaaacccatcacagaaatggacagcttgggtctgtaacaaagcattcatgttttagagcataggtcagtaattgtatatgagagcatacactgctacatacaaattaactgatcagaccacaacttttcaatgtttaaaacagaataagcttccctgtaaaagcagcacctttgtgacgttttaactttagtattcctctccttcttcctcaccctctccttcaacagaatccacaccaacctcctcataatccttcctcgcagcacatgaatcacaggtattcctactgcaagcgggaggcggaggagcgggaagcggcggagcgcgaggcgcgcgagaaagggcacttggaacccaccgagctgctgatgaaccgggcttacttgcagagcattacccctcaggggtactctgactcggaggagagggagagtatgccgagggatggcgagagcgagaaggagcacgagaaagaaggcgaggatggctacgggaagctgggcagacaggatggcgacgaggagttcgaggaggaagaggaagaaagtgaaaataaaagtatggatacggatcccgaaacgatacgagatgaagaagagactggagatcactccatggacgatagttcggaggatgggaaaatggaaaccaaatcagaccacgaggaagacaatatggaagatggcatgtaataaactactgcattttaagcttcctatttttttttccagtagtattgttacctgcttgaaaacactgctgtgttaagctgttcatgcacgtgcctgacgcttccaggaagctgtagagagggacagaaggggcggttcagccaagacagatgtagacggagttggagctgggtattgttaaaaactgcattatgcaaaaattttgtacagtgttaaggcctaaaaactgtgtggttcagagactaattcctgtgtttaatagcatttatactttaagcacaactagaaaattgtaagaattgcactctacttatgtatcactacaaactttaaaaaactatgtctaatttatattaatacattttaaaaaggtgcccgcactaccatacatcagtatttttattattattattgttattcctttttaatttaatgtgctcgcactacaatgcatcagtattatgattcctctgtactttcctttcgctattcatcaatttcccattttttttttcagcttaagtaaccacacaattttaggcctcaatttttttttttctgtgaaggaacttgaagtgatgcatgtgtgaatttaagataccgaagtcttaaagtgacctggacgtgaaggaaaaagtaagatgagaaataaagaaagcctttgtaaggtggttttaaaagccttatatgcaaaccttttaatctgtgtttctgcaagtgccatccttgtacagtgttaagagggtaacatgggttacctttgcaccagcttcagtgttaagctcaccctgttctttgaagcacccatgtcagtattagaagaataggcagcagttccttagtttacatatgtttgtgcaattattttctgtacttttttgttcattaattttgtcagtattacaccaaactgtttttgcaacaaaaaaattttttttgcattcatttaattttaggtcaaataacattttatttatgtggctcattttatatttcctaattttatttatttcatactgtagtgtacagtattatagttcttcaatatatagatatattttagtaaaaaaggaacatgacgttgatcatttgggcaaattttacgtaaagagaagagcatttattgtgttttggaacattaattgtgagatgggatttttcaattttattattttatttttgtttttttccaattactggaaattccaaatttgggaacttttgatacgatcttgtgaaaacactgtattttcgactgaaaattccactttcttcatcttgttttttagctaaaaagagggactgttaaatacaatgtatgataccatgacaaaaatctttcctgaattgtctttgtaaaagtattattgaattttcaatttgtaatttcttttgaaaatgaccatgctcgaataaaaatgtagccaaactaaaaaaaaaaaaaaaa

SEQ ID NO: 59 - protein, 141 aa, Homo sapiens
MNHRYSYCKREAEEREAAEREAREKGHLEPTELLMNRAYLQSITPQGYSDSEERESMPRDGESEKEHEKEGEDGYGKLGRQDGDEEFEEEEEESENKSMDTDPETIRDEEETGDHSMDDSSEDGKMETKSDHEEDNMEDGM

SEQ ID NO: 60 - DNA, 2168 bp, Homo sapiens
gcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgcatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccatcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactgaagccaggcgccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtccccaaaaaaaaaaaaaa

SEQ ID NO: 61 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCHPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 62 - DNA, 430 bp, Homo sapiens
taaagatctgtgttcagagtcatactgaayagagacttctggactctatagaacccactgcctcctgatgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccgtttctcccactggttgaacattccagcctctgtctcctgctctagg

SEQ ID NO: 63 - protein, 121 aa, Homo sapiens
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTRFSHWLNIPASVSCSR

SEQ ID NO: 64 - DNA, 112 bp, Homo sapiens
  unsure (8)
  unsure (12)
  unsure (36)
  unsure (41)
  unsure (44)
tttcctgntttnggatccccgattcattaaagcaanggggnttnaaaaaaaaaaaaaaaaaaaaaaaaaaaaaaaaaaaaaaaaaaaaaaaaaaaaaaaaaaaaaaaaaaaa

SEQ ID NO: 65 - DNA, 324 bp, Homo sapiens
  unsure (1)
  unsure (69)
  unsure (74)
  unsure (125)
  unsure (159)
nctaccccaacctgtgtggctgggccgcggtctcccctcaagggcctggggccgtgcctcgggtgtacncgtangggtctgtgtgctgggggtggctcaccgggcagcgtgggtgagcggcgcancggcggcagcggagaacgagagaggggagcaganacagaatcgcctaagctgaagtgtattggcgccatcatggctcactgcggcctccggctccttggctcgggtgattctcctgcctgagcctccctagtagctaggactacagtgctgtagaagaaaatcacatgattggtgccctcaaaaaattggtgccacttg

SEQ ID NO: 66 - DNA, 794 bp, Homo sapiens
  unsure (61)
  unsure (82)
  unsure (108)..(120)
  unsure (184)
  unsure (754)
cattatttcatcaccagagaatacacatgcagcaaatagcattgtgagtcaaactattccnaaagcacagattcagcaatcnacacacactcatctggatatctcacnnnnnnnnnnnnnttaactgatgaaaaaagtaatggaacaattgcccttgtggatgattctgaggatcctggagccnatgtatctaacatacagcttcagcaaaaaatttcaagtctggagattaaactcaaagtatctgaagaagaaaaacagagaattaaacaggatgtggaakcattgatggaaaagcataatgtcttagaaaaaggctttctaaaagaaaaagagcaagaggccatttcttttcaagatagatacaaagaacttcaggaaaaacataaacaagaattggaagacatgaggaaagctggtcacgaagccctcagcattattgtggatgaatataaggcactactgcagtcttcagttaagcaacaagtagaagctattgaaaaacagtacatttctgcaattgagaaacaggcacacaagtgtgaggagttgctaaatgctcagcatcagaggctccttgaagtgctagatacagagaaggaactgttaaaagaaaaaataaaggaagctttgattcagcaatctcaagaacagaaggaaatattggaaaagtgtttggaggaagaaaggcaaagaaataaagaggcattagtatccgctgcaaagcttgaaaaagaaccagtgaaggatgcanttttaaaattcgtagaagaagaaagaaaaaaaaaaaaaaa

SEQ ID NO: 67 - protein, 164 aa, Homo sapiens
  UNSURE (156)
MEKHNVLEKGFLKEKEQEAISFQDRYKELQEKHKQELEDMRKAGHEALSIIVDEYKALLQSSVKQQVEAIEKQYISAIEKQAHKCEELLNAQHQRLLEVLDTEKELLKEKIKEALIQQSQEQKEILEKCLEEERQRNKEALVSAAKLEKEPVKDAXLKFVEEER

SEQ ID NO: 68 - DNA, 1494 bp, Homo sapiens
cgtttgtcgacgccgctgccaccgcctgcctgagagaagtcgtcgcggccgaccccgtcgcctccgccggctaccatgtccgcccaggcgcagatgcgggccctgctggaccagctcatgggcacggctcgggacggagacgaaaccagacagagggtcaagtttacagatgaccgtgtctgcaagagtcaccttctggactgctgcccccatgacatcctggctgggacgcgcatggatttaggagaatgtaccaaaatccacgacttggccctccgagcagattatgagattgcaagtaaagaaagagacctgttttttgaattagatgcaatggatcacttggagtcctttattgctgaatgtgatcggagaactgagctcgccaagaagcggctggcagaaacacaggaggaaatcagtgcggaagtttctgcaaaggcaggaaaagtacatgagttaaatgaagaaataggaaaactccttgctaaagccgaacagctaggggctgaaggtaatgtggatgaatcccagaagattcttatggaagtggaaaaagttcgtgcgaagaaaaaagaagctgaggaagaatacagaaattccatgcctgcatccagttttcagcagcaaaagctgcgtgtctgcgaggtctgttcagcctaccttggtctccatgacaatgaccgtcgcctggcagaccacttcggtggcaagttacacttggggttcattcagatccgagagaagcttgatcagttgaggaaaactgtcgctgaaaagcaggagaagagaaatcaggatcgcttgaggaggagagaggagagggaacgggaggagcgtctgagcaggaggtcgggatcaagaaccagagatcgcaggaggtcacgctcccgggatcggcgtcggaggcggtcaagatctacctcccgagagcgacggaaattgtcccggtcccggtcccgagatagacatcggcgccaccgcagccgttcccggagccacagccggggacatcgtcgggcttcccgggaccgaagtgcgaaatacaagtaactactctgactccttcggtagctgcaaccaggagttctccagagagcgggcatccagagaggagtcctgggagagcgggcggagcgagcgagggcccccggactggaggcttgagagctccaacgggaagatggcttcacggaggtcagaagagaaggaggccggcgagatctgaacccgtctcccgggtgctgtaaatagtctgataaacgttcacacagtctaaaattaccctttatatttgctgaatacaactcatcttttgtagtttaaaatttctattgttttggagctagctgtgagtttctagaagtgtacagagttgctcctgtgttcccgggtcatgttgagtaggaataaataaatctgatgctgcctcctggaaaaaaaaaaaaaaaaaaaaaaaaaaaaaaaaaaaaaaaaaaaaaaaaaaaaaaaaaa

SEQ ID NO: 69 - protein, 325 aa, Homo sapiens
MSAQAQMRALLDQLMGTARDGDETRQRVKFTDDRVCKSHLLDCCPHDILAGTRMDLGECTKIHDLALRADYEIASKERDLFFELDAMDHLESFIAECDRRTELAKKRLAETQEEISAEVSAKAGKVHELNEEIGKLLAKAEQLGAEGNVDESQKILMEVEKVRAKKKEAEEEYRNSMPASSFQQQKLRVCEVCSAYLGLHDNDRRLADHFGGKLHLGFIQIREKLDQLRKTVAEKQEKRNQDRLRRREEREREERLSRRSGSRTRDRRRSRSRDRRRRRSRSTSRERRKLSRSRSRDRHRRHRSRSRSHSRGHRRASRDRSAKYK

SEQ ID NO: 70 - DNA, 1761 bp, Homo sapiens
caagggaagttctgagggctgagaggttgctcattcgtcagagcgtgctgcccaccctccacccctgcatggcagaaactgtgcaggggacgaggccaaggaatcaggagacccagaggcaggggtggcccggagacggtgaagaaaccaagacgcagagaggccaagccccttgccttgggtcacacagccaaaggaggcagagccagaactcacaaccagatccagaggcaacagggacatggccacctgggacgaaaaggcagtcacccgcagggccaaggtggctcccgctgagaggatgagcaagttcttaaggcacttcacggtcgtgggagacgactaccatgcctggaacatcaactacaagaaatgggagaatgaagaggaggaggaggaggaggagcagccaccacccacaccagtctcaggcgaggaaggcagagctgcagcccctgacgttgcccctgcccctggccccgcacccagggccccccttgacttcaggggcatgttgaggaaactgttcagctcccacaggtttcaggtcatcatcatctgcttggtggttctggatgccctcctggtgcttgctgagctcatcctggacctgaagatcatccagcccgacaagaataactatgctgccatggtattccactacatgagcatcaccatcttggtcttttttatgatggagatcatctttaaattatttgtcttccgcctggagttctttcaccacaagtttgagatcctggatgccgtcgtggtggtggtctcattcatcctcgacattgtcctcctgttccaggagcaccagtttgaggctctgggcctgctgattctgctccggctgtggcgggtggcccggatcatcaatgggattatcatctcagttaagacacgttcagaacggcaactcttaaggttaaaacagatgaatgtacaattggccgccaagattcaacaccttgagttcagctgctctgagaaggaacaagaaattgaaagacttaacaaactattgcgacagcatggacttcttggtgaagtgaactagacccggaccagctcccctcaaaaagaagacactgtctcatgggcctgtgctgtcacgagaggaacagctgcccctcctgggccgcttggtgagaggtttggtttgatacctctgcctccctcctgccagcatggattctgggtggacacagccttgtggaaggtccagtaccaccaagagctgcccatccactcccaccccacactgtatcaaatgtatcacattttctcatgttgaacactttagccttaattgaaaatgagcaacaaagctggacaattgctagttgtatataaaatttaatctcaccgaatgtacagttttcaaatttcacgtgtatattaaggaactgatgcatctgagcattctgaaagaaagaaaaagaagctactttagctgccaccccattctagaaaagtctcttattttcaagctgttctaaatagcttcgtctcagtttccccaaaaggggtacccaggcccctcctctgtgtgccccagctgcatcagccagcttctaggtggctccattgttttctgccacctgacaacatttttcctcaattactgtacaactactgtataaaataaaacaactactgtataaaataaactctctcttttccctggaaaaaaaaaaaaaaaaaaaaaaaaaaaaaaaaaaaaaaaaaaaaaaaaaaaaaaaaaaaaaaaaaaaa

SEQ ID NO: 71 - protein, 273 aa, Homo sapiens
MATWDEKAVTRRAKVAPAERMSKFLRHFTVVGDDYHAWNINYKKWENEEEEEEEEQPPPTPVSGEEGRAAAPDVAPAPGPAPRAPLDFRGMLRKLFSSHRFQVIIICLVVLDALLVLAELILDLKIIQPDKNNYAAMVFHYMSITILVFFMMEIIFKLFVFRLEFFHHKFEILDAVVVVVSFILDIVLLFQEHQFEALGLLILLRLWRVARIINGIIISVKTRSERQLLRLKQMNVQLAAKIQHLEFSCSEKEQEIERLNKLLRQHGLLGEVN

SEQ ID NO: 72 - DNA, 928 bp, Homo sapiens
  unsure (367)
  unsure (448)
  unsure (467)
  unsure (508)
  unsure (539)
aatcgggagtcctggaaagttaattccaatgtcatgacgtcaaacttaaaatggtcgtctgccaccatgaagattgataaccctcacataacatatactgccagggtcccagtcgatgtccatgaatacaacctaacgcatctgcagccttccacagattatgaagtgtgtctcacagtgtccaatattcatcagcagactcaaaagtcatgcgtaaatgtcacaaccaaaaatgccgccttcgcagtggacatctctgatcaagaaaccagtacagcccttgctgcagtaatggggtmtatgtttgccgtcattagccttgcgtccattgctgtggtactttgccaaaagatttaagagaaaaaantaccaccactcattaaaaaagtatatgcaaaaaacctcttcaatcccactaaatgagctgtacccaccactcattaacctntgggaaggtgacagcgagnaagacaaagatggttttgcagacaccaagccaacccaggtngacacatccagaaggtattacatgtggtaantcagaggatattttgcttctggtagtaaggagcacaaagacgtttttgctttattctgcaaaagtgaacaagttgaagacttttgtatttttgactttgctagtttgtggcagagtggagaggacgggtggatatttcaaatttttttagtatagcgtatcgcaagggtttgacacggctgccagcgactctaggcttccagtctgtgtttggtttttattcttatcattattatgattgttattatattattattttattttagttgttgtgctaaactcaataatgctgttctaactacagtgctcaataaaatgattaatgacaggatggggttcccctgtgcttttaccagtagcatgacccttcctgaagccatccgtagaaag

SEQ ID NO: 73 - protein, 52 aa, Homo sapiens [sequence omitted]

SEQ ID NO: 74 - DNA, 49 bp, Homo sapiens
  unsure (9)
aaattaaanaaaaaaaaaaaaaaaaaaaaaaaataaagaaaaaaaaaaa

SEQ ID NO: 75 - DNA, 597 bp, Homo sapiens
attctacaagataacttcccagtactttaaaaaagtctcaaagtcataaacaagaaagaactgagggactattgcatattggagcgatctaaagaagtattacaatttgtggaattcttgattaaatcctggaccagcaaaaggacattagtgggaaaattgatgaaattcaaatgagatcttatattgaagttaattgtgtcagtgtacatttcctggttttcataattgcaagtgattatgtaaggtttgttaatattaggagcagctgggtaaaggttatacaaaaactctatactatttttgcatttttttctgtaagtttaaaacattttccaactaaaaagttgaaaacacatgtattagagacacatgcgtatgtgtctctaataatcttaaatatatttaagatgatagaaggaattcttgagatagtaaaatgaagtcaccaaaaaacaaacaaagaaacaaaacgaaatcaccaaaatctatcaataaatttcaggtaatacttttggcagattcattcctttgagatggagtctcactcccagtctgggcaacgagcgaaactccgtctaaaaaaaaaaaaaaaaa

SEQ ID NO: 76 - protein, 89 aa, Homo sapiens
MRSYIEVNCVSVHFLVFIIASDYVRFVNIRSSWVKVIQKLYTIFAFFSVSLKHFPTKKLKTHVLETHAYVSLIILNIFKMIEGILEIVK

SEQ ID NO: 77 - DNA, 1804 bp, Homo sapiens
  unsure (1794)
ggagttcattaggccagtggcactgatttttccttcccatcatagctatttattctagtaagaggataataaaagaaatttcctatacaagaactgaaattttccatttgtatggatctagtcactttcagatttcaatttgaggttaagtatataaagcacatcccaattttatatgctgccttgagaaaattacaggatgcacggcaatttgtaggaatttcaaatgggatcatttaaacatttgaaaaattattttaaaaaccatctagtttgcttttggattttagacattaaagcctatgttgtcttgttaacaggggtggaatgtataaccatcagattcagcatgtgatttcacctttgaatctgagtatttcttccctatcttctttgagtcatttttggagcagactgtcaccagtattgataactaagcattaaagggaaaagttgcattgcaactatgcattggtttcctggaagaacttttcttttgttttagtgaatgaagaggcttgatgggatcacttactgtaactccttctacataaggaccccttctgcaagcagaacacaaaagaacatgctcaaggagtatcccattttctggataaattgaagaagtttgctagtaatgtctttatactagcgtcttccttgtatccctttgctggcaagggaatacaaggcgtcaagaccacagatcaaaacaccccacatttgagtggagtcttatttttactccaagagcagttattcccttctagtctaaaattggcagttttttcttttttttaataaaatttttaaaatattcccaaaccagtggaacacagacactggctgcacttagtactgccaaaagccaaggtcatttgcacatattccatcaacctgtcgagaattaggcctcactttataacccaaggcatggaagtgcatgcattctcttagctgggcaaacaattatactgtagttgtgatacaacacatgtggcttttatttgtactgcacatatccactgtacagccacttgggagtatcgtggttagcttgcagcaactgctgtctgcatttatactgtttattgcatattcttttccctggaagtgaaagagaaatgtttttcttgttgcattgattacattttataaatttgcttagctggaaagtttgggaaaagaggcctgtttgtcaattgtacaaccgattgtgaagctctagtgtgaatatttttacgtctgtattagacattttctttgcaaatctattgttcgattgaaatgtaaatgaaattaaagatggtgtacacccatcatgtaaaaagcaggcaccatctctaagatggatttaatgctcatttttaaggcatatactcagcttctatttaaaactataatttaaaataattctgtacaatgaaatggggaatatatatgggaataaattctattccatttatttcaatttgaatttccaaattgtaatgtttccctttgtgctataggaataggattaaatgggggaagactaggatttataaggcctgtatatggggggagggcagagatggaacaatgagggttgtgatgatagtgaatagcaaagagtgaattctgtgtgtttttgctgtagcactgaagtgaagagatattagctttggctgttcacaaaatagagcatcatgattttcagtgtttgagagaaaattgatggaaaaagtttgcagtacttgacatgtatttgcatgcacaaaataaaattatttgtccaccttaaaaaaaaaaaanaaaaaaaaaa

SEQ ID NO: 78 - protein, 37 aa, Homo sapiens
MKRLDGITYCNSFYIRTPSASRTQKNMLKEYPIFWIN

SEQ ID NO: 79 - DNA, 360 bp, Homo sapiens
agaataccaagactgtgtgtacacgcagatgtcagtggcagagaatgaagatcagcttcgtgcaaagggttatgacaaaacaccagacttcattttacaagtaccagttgctgtagaagggcacataattcactggattgaaagcaaagcctcatttggtgatgaatgtagccaccacgcctacctgcatgaccagttctggagctactggaatagggtcccaatataacagacaaatggtgaaacagagggatactcactaggaaacagatttgggccaggcttagtcatctattggtatggatttatccaggagctggactgcaaccgggaaaggggcatcctgctcaaagcctgttt

SEQ ID NO: 80 - protein, 20 aa, Homo sapiens
MDLSRSWTATGKGASCSKPV

SEQ ID NO: 81 - DNA, 202 bp, Homo sapiens
  unsure (136)
  unsure (138)
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaantnaaaaaaaaaaaaaaaaaaaaaaaaaaaaaaaaaaaaaaaaaaaaaaaaaaaaaaaaaaaaaaaa

SEQ ID NO: 82 - DNA, 1189 bp, Homo sapiens
  unsure (1155)
ggccttcatggcctaatcagattttagtggcaagacaggtgatgatagggagaaagaaagagtcctgcctgtcaaaagtggctgtgctatataratgaaccctcacaggtagcacctctcagagagaataratggtaaatgtttcttttcaggtctttaaaagtgtcaggctatcagttaacctctcctagatctcagaaatgcctagaaagagaagtcctggctacatcaatggaaattctccacagatgcaaattttctctacaaaagatggctttgcagagccacctcagtctgttgtccctgtagcagccatttcaaattatgtcaaagagatatattttggggtaaaatattttgattatcttcattagtatatctcaattttgtcaatacaaacctgagagttatagtcagaggttgaattttcatttcaaaatgttttcctagttttttttctcttttttgttttattgtaagttgacaatttataattgtataaaagtatgaggtacaaagtgatgttatagcttaagaatacagtatggtatgattaaatcaagttattaacctatccttcacgttaaatgcttaaattttttgatgagaacatttgaaatttactcttggaaggtaaaaaaaaatctcaggaccccccaaattaaagccatgaagctgaattgtgcaacatcctcttccaaatggaagcttgtcttccaggtacagaacaaaaacaagactcatttcttcacctgcctaaagatgtgcacataattggctcctcctttactccctttttctcttctaacattcattatatcttgtgtaaaatgtagatttactggacactaactaaaatttcacagggttgtacccatttgccttactgcctacctacctgtyttcctacgtaccttytccccactttaaggaatgcatacatattaaacctcccaaaaacytytttagaaaaatagccacaggtttatctgtggctggtgtgtttccctagatgcactytaaagctggcttaataaacctcagtgattgaaacttatgcctcaatcactcattttggttgtcactgtcttaccaatttgaaatgtaaaatattytactactaattatatttaccacattgtgcaacagaactcnaaaaaaaaaaaaaaaaaaaaaaaaaaaaaaaaaa

SEQ ID NO: 83 - protein, 56 aa, Homo sapiens
MRTFEIYSWKVKKNLRTPQIKAMKLNCATSSSKWKLVFQVQNKNKTHFFTCLKMCT

SEQ ID NO: 84 - DNA, 525 bp, Homo sapiens
cataacagcgtcagagagaaagaactgactgaaacgtttgagatgaagaaagttctcctcctgatcacagccatcttggcagtggctgttggtttcccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatyttytttaataaacatgraagcaaagraaaaaaaaaaaaaaaaaaaaaaaaaaaaaaaaaaaaaaaaaaaaaaaaaaaaa

SEQ ID NO: 85 - protein, 85 aa, Homo sapiens
MKKVLLLITAILAVAVGFPVSQDQEREKRSISDSDELASGFFVFPYPYPFRPLPPIPFPRFPWFRRNFPIPIPESAPTTPLPSEK

SEQ ID NO: 86 - DNA, 349 bp, Homo sapiens
  unsure (9)
  unsure (159)
  unsure (188)
  unsure (230)
  unsure (232)
  unsure (270)
acttcgcancccatcccggctggacgcgaccggggagtgcagcagcccgttcccctcctcggtgccgcctctgcccagcgtttgcttggctgggctaccacctgcgctcggacggcgctcggagggtcctcgcccccggcctgcctacctgaaaaccanaactgatggctctatttgcagtctttcanacaacattcttcttaacattgctgtccttgaggacttaccanantgaagtcttggctgaacgtttaccattgactcctgttntcacttaaagtttccaccaattctacgcgtcagagtttgcacttacaatggactgtccacaaccttccttatcatcagg

SEQ ID NO: 87 - DNA, 563 bp, Homo sapiens
  unsure (63)
  unsure (83)
  unsure (116)
  unsure (177)
  unsure (183)
  unsure (228)
  unsure (240)
tccattccagcaccagccaacagcacaaaattaatccttgacaggtgttcctaccaaatttgngtcatagccaacaacagtgngggtgcttctcctgcttctgtaatagtcatttntgcagaccccgaaaacaaagaggttgaggaagaaagaattgcaggcacagagggtggattntttttnttttggaaaccccaacctggagatgttataggttatgttgtggantggtgtgaccanacccaggatgtgcctcggtgatttccagtggaagaatgtaggtcccaataccacaagcacagtcattagcacagatgcttttaggccaggagttcgatatgacttcagaatttatgggttatctacaaaaaggattgcttgtttattagagaaaaaaaacaggatactctcaggaacttgctccttcagacaaccctcacgtgctggtggatacattgacatcccactccttcactctgagttggaaagattactctactgaatctcaacctggttttatacaagggtaccatgtctatctgaaatccaaggcgaggcagtgc

SEQ ID NO: 88 - protein, 58 aa, Homo sapiens
RKKTGYSQELAPSDNPHVLVDTLTSHSFTLSWKDYSTESQPGFIQGYHVYLKSKARQC

SEQ ID NO: 89 - DNA, 361 bp, Homo sapiens
  unsure (102)
  unsure (105)
  unsure (153)
  unsure (186)
  unsure (191)
  unsure (252)..(253)
gaaaaggcagttctttcagatggttcagaatgttgcaaatacaaaattgacaacccggaagaaaaggcattgattgtggacaacctaaagccagaatccttntangagtttttcatcactccattcactagtgctggtgaaggccccagtgcnacgttcacgaaggtcacgactccggatgaacantcctngatgctgattcatatcctactgcccatggttttctgcgtcttgctcatcatggtcatgtgnnacttgaaaagtcagtggatcaaggagacctgttatcctgacatccctgacccttacaagagcagcatcctgtcattaataaaattcaaggtaaaaaaaaaaaaaaaaa

SEQ ID NO: 90 - DNA, 756 bp, Homo sapiens
  unsure (37)
  unsure (54)
  unsure (376)
  unsure (433)
attgaattctagacctgcggccgctgctccctcagangcccctgatgtctgganaattgtgagcttggagccaggaaatcatactgtgaccttattctggaagccattatcaaaactgcatgccaatggaaagatcctgttctataatgtagttgtagaaaacctagacaaaccatccagttcagagctccattccattccagcaccagccaacagcacaaaactaatccttgacaggtgttcctaccaaatctgcgtcatagccaacaacagtgtgggtgcttctcctgcttctgtaatagtcatytctgcagaccccgaaaacaaagaggttgaggaagaaagaattgcaggcacagagggtggattttttttnttttggaaaccccaacctggagatgttataggttatgttgtggactggtgtgaccanacccaggatgtgcctcggtgatttccagtggaagaatgtaggtcccaataccacaagcacagtcattagcacagatgcttttaggccaggagttcgatatgacttcagaatttatgggttatctacaaaaaggattgcttgtttattagagaaaaaaaacaggatactctcaggaacttgctccttcagacaaccctcacgtgctggtggatacattgacatcccactccttcactctgagttggaaagattactctactgaatctcaacctggttttatacaagggtaccatgtctatctgaaatccaaggcgaggcagtgc

SEQ ID NO: 91 - protein, 58 aa, Homo sapiens; identical to SEQ ID NO: 88

SEQ ID NO: 92 - DNA, 79 bp, Homo sapiens
tcattaataaaattcaaggtaaatgttaaaaaaaaaaaaaaaaaaaaaaaaaaaaaaaaaaaaaaaaaaaaaaaaaaaa

SEQ ID NO: 93 - DNA, 1939 bp, Homo sapiens
attggttttcaaatattaaaccagcattttattttaaaataaaccccacttagttatgatattttatcttttttatataatgtgggatttgatttgccgatattttattaaagatttttgcatcatgttctttttctgaaattttattttgttctaatgtcataaaataagttggaaattatcctctctatctctactttgtgaaagaatttatattacattggtattatttcttccttgaatgtttgatagactatactagtgaaagcatctgggcctagggttttctctgtggaagattttcagttacaaattcaattttactgagtcaggtttgataagttacatttctcaaggaatttatctatttaatcattgaatgtattgaacattcatttgtttataattttgttttgttattgaaaatgtctgtaagatttatagtgatgttcccttttctattcctgacattgttaatttgtgttctctctccctccatccctctctcactctctcttgtcagctggtctgtgggattatcacctttattaatcttttcagagaaccactttttatttctttgatttcctttattgtttgttaactttttagtttattgattccctctcttagctttattacttccttccttctacttagtaagagtttaatttgccctcatttttttagcttcttaaggtaggaaatttgattatattttaaacttaaaaaaacttaaagctataaaattccctttaagcactgttttagctgaattctacatattttgattgggtatgttttcatcattcaattaaaaacatagtttctaattttttcttgcaatttcttctttaacccataggttatttggcagtttgctgctgaatttcaaagtgcttgggaggttttatagctatcttgttaaaaattgatttctaatttaattttgtcagagaacatactttgtatgacttaaatcttttaaaatacgttcagactgatttagggtccagcatatggtacgtcttagtgaatgtaatatatgccattggaaatgatgcacattctgcaattgttggttgtaatgtgctataaatgtcaaccaggttaacttggttgatagtattgttgaattcctgtgttccaaatgatattttttgtctgtttattctatcaattaatgagaaaggtttattaaaatctccaaccattgttgtgcatttatttctatttctttagttctgttgatttttgttttatatattttgaagctctgagtacatctccctgatgagttgacccttttatcattatgaaatgtcattctttctctgtaatgcttatcttaaaatctactttgtttgatatgaatgtagccacttaagctttcttatgcatactgtttgcaaagtgtgtgtgtgtgtgtgtgtgtgtgtgtgtatctatctcaatcctttagtttccgcctatctctgtctttttatttaaagtgtgtttttgcaggtagtgtataacttggatcttgtttttatattcagtctgacaatttatgcttcttgattattgtgtttagtttatttgcatagtgtgtagtccattgacatttggcataatattgataatttgtgttggagcgtattgtcttcatttctgattgtcccatctggttttgttgctgttttgttgttcctctgttccacactttttgtcttcttttgggttaatcaaatattctttggctttgcatcttatgtcttctgttggatttttatctataccttgtcgtatttttctttagtggtttctctaggggttacactatacattattaattaatcatagtccatttagaattaatattgtacactttacataaaatgtaaaacatttgtcacagtataaagtctattccttccaaaaaaaaaaaaaaaaaaaa

SEQ ID NO: 94 - protein, 52 aa, Homo sapiens
MSVRFIVMFPFLFLTLLICVLSPSIPLSLSLVSWSVGLSPLLIFSENHFLFL

SEQ ID NO: 95 - DNA, 1252 bp, Homo sapiens
gtagaaatcccaaaaagatttggacagaggcagagcctatgtcactacaccttaaaggataacaaggtttatatcaagactcatggtgaacatgtaggttttagaattttcatggatgccatactactttctttgactagaaaggtgaagatgccagatgtggagctctttgttaatttgggagactggcctttggaaaaaaagaaatccaattcaaacatccatccgatcttttcctggtgtggctccacagattccaaggatatcgtgatgcctacgtacgatttgactgattctgttctggaaaccatgggccgggtaagtctggatatgatgtccgtgcaagctaacacgggtcctccctgggaaagcaaaaattccactgccgtctggagagggcgagacagccgcaaagagagactcgagctggttaaactcagtagaaaacacccagaactcatagacgctgctttcaccaactttttcttctttaaacacgatgaaaacctgtatggtcccattgtgaaacatatttcattttttgatttcttcaagcataagtatcaaataaatatcgatggcactgtagcagcttatcgcctgccatatttgctagttggtgacagtgttgtgctgaagcaggattccatctactatgaacatttttacaatgagctgcagccctggaaacactacattccagttaagagcaacctgagcgatctgctagaaaaacttaaatgggcgaaagatcacgatgaagaggccaaaaagatagcaaaagcaggacaagaatttgcaagaaataatctcatgggcgatgacatattctgttattatttcaaacttttccaggaatatgccaatttacaagtgagtgagccccaaatccgagagggcatgaaaagggtagaaccacagactgaggacgacctcttcccttgtacttgccataggaaaaagaccaaagatgaactctgatatgcaaaataacttctattagaataatggtgctctgaagactcttcttaactaaaaagaagaatttttttaagtattaattccatggacaatataaaatctgtgtgattgtttgcagtatgaagacacatttctacttatgcagtattctcatgactgtactttaaagtacatttttagaattttataataaaaccacctttattttaaaggaaaaaaaaaaaaaaaaaaaaaaaaaaaaaaaaaaaaaaaaaaaaaaaaaaaaaaaaaa

SEQ ID NO: 96 - protein, 289 aa, Homo sapiens [sequence omitted]

SEQ ID NO: 97 - DNA, 492 bp, Homo sapiens
ctaggttctgggaagatggcgaaggtctcagagctttacgatgtcacttgggaagaaatgagagataaaatgagaaaatggagagaagaaaactcaagaaatagtgagcaaattgtggaagttggagaagaattaattaatgaatatgcttctaagctgggagatgatatttggatcatatatgaacaggtgatgattgcagcactagactatggtcgggatgacttggcattgttttgtcttcaagagctgagaagacagttccctggcagtcacagagtcaagcgattaacaggcatgagatttgaagccatggaaagatatgatgatgctatacagctatatgataggattttacaagaagatccaactaacactgctgcaagaaagcgtaagattgccattcgaaaagcccaggggaaaaatgtggaggccattcgggagctgaatgagtatctggaacaatttgttggagaccaagaagcctggcat

SEQ ID NO: 98 - protein, 159 aa, Homo sapiens
MAKVSELYDVTWEEMRDKMRKWREENSRNSEQIVEVGEELINEYASKLGDDIWIIYEQVMIAALDYGRDDLALFCLQELRRQFPGSHRVKRLTGMRFEAMERYDDAIQLYDRILQEDPTNTAARKRKIAIRKAQGKNVEAIRELNEYLEQFVGDQEAWH

SEQ ID NO: 99 - DNA, 85 bp, Homo sapiens
  unsure (20)
  unsure (27)
ccttgttaaataaaccatgntgatttnttaaacttgccaaaaaaaaaaaaaaaaaaaaaaaaaaaaaaaaaaaaaaaaaaaaaaa

SEQ ID NO: 100 - DNA, 313 bp, Homo sapiens
  unsure (68)
  unsure (108)
  unsure (137)
  unsure (288)
ccttggattatctgaactaaaaattggacagattgatcagctggtaaaaaatctacttcctggatttngtaaaggcaaaaacatttcttcccattggcatacatcccntgtctctgcacaatccttctttgaaaatnaatatggtaacttaaatatatttagtacattacgttcctcttgcttgtatcgacatcattcaaaagctcttcaaagcatttgttcaaatcttcagtactggccagttttcatacagtctcggggttttaaaactttgaaatcaaggacacnacgtctccagtctacctccgagaga

SEQ ID NO: 101 - DNA, 964 bp, Homo sapiens
  unsure (395)
gcttttccggagcagagttggagaatcttgtgaaccaggctgcattaaaagcagctgttgatggaaaagaaatggttaccatgaaggagctggagttttccaaagacaaaattctaatggggcctgaaagaagaagtgtggaaattgataacaaaaacaaaaccatcacagcatatcatgaatctggtcatgccattattgcatattacacaaaagatgcaatgcctatcaacaaagctacaatcatgccacgggggccaacacttggacatgtgtccctgttacctgagaatgacagatggaatgaaactagagcccagctgcttgcacaaatggatgttagtatgggaggaagagtggcagaggagcttatatttggaaccgaccatattacnacaggtgcttccagtgattttgataatgccactaaaatagcaaagcggatrgytaccaaatttggaatgagtgaaaagcttggagttatgacctacagtgatacaggggaaactaagtccagaaacccaatctgccatcgaacaagaaataagaatccttctaagggactcatatgaacgagcaaaacatatcttgaaaactcatgcaaaggagcataagaatctcgcagaagctttattgacctatgagactttggatgccaaagagattcaaattgttctkgaggggaaaaagttggaagtgagatgataactctctkgatatggatgcttgctggttttattgcaagaatataagtagcattgcagtagtctacttttacaacgctttcccctcattcttgatgtggtgtaattgaagggtgtgaaatgctttgtcaatcatttgtcacatttatccagtttgggttattctcattaggacacctattgcaaattagcatcccatggcaaatatattttgaaaaaataaagaactatcaggattgaaaaaaaaaaaaaaaaaaaaa

SEQ ID NO: 102 - protein, 166 aa, Homo sapiens
  UNSURE (125)..(126)
MVTMKELEFSKDKILMGPERRSVEIDNKNKTITAYHESGHAIIAYYTKDAMPINKATIMPRGPTLGHVSLLPENDRWNETRAQLLAQMDVSMGGRVAEELIFGTDHITTGASSDFDNATKIAKRXXTKFGMSEKLGVMTYSDTGETKSRNPICHRTRNKNPSKGLI

SEQ ID NO: 103 - DNA, 1362 bp, Homo sapiens
cttttcatatccttttattttcccaattcatataggaaagataagtattttccctttcattttcaaaattgattggtacactaacctcctccatagtaactaatttgtatttttttattatgaactcatgaatttaaacatttgatgttgtttcagtccattgcagtcattatccttattgattctcaaagtgttttatttttgacaaataagttcttcaaattagttagttattttgatagtgaccttaatagtctttgctagcttccatccctgtatggcataacaaaacattccaggcccctttagtacatttcctgtcccagacctggtattagtcatctaatctggaagccctgctttctttctgtgggaaacattatttcaagacattagggataagaatgccagttgctactgagttggttattgtttcaaggatttatcaatacatagagcaaataattatgttttgctttgtcttatttttatttctttactttagaaacagtacagctacttacaaatcaagtttagaactctcaggttatcttaaatctgaagcttctaccttcctaagaacaaaacaccggaatgatgagatgtcatataaatatccatttattttatttcacaatacatacattgacctattgtatgtatgattagtacaaatggtttaaattggcttgttgtgctttgttttgtttttgcagtttttttttgtcattagattttatctcactagaaatgtatagtcaaatttctgtgttctaaagtcacttgaagtagtttttctgtttgtagtaatgttaccagttggacacattaggttcatttatttttgggaattgcttttgttcccatttaaatttttatttatcttgcgtatcttggattaacggtaatagaccatatacaattttctctactttttttcattkgagagtttcctcatttattttgttttcggatgcaaagtatttcattgtatgaattaccagtttgttcaattagttttctttgataacatttgggttgcttctagtcttttgttatwacagagtcttgcaatgaatagtcttatgcttatatgttttgtggtttctggcaaggatttcaacgtaatttaaggctatcctctacttaagttatggccaattaagtgtctgattaaatgtctgtaataatttaaaaataacaatacttaatttaaaagagcttataatacataattattcattagagagatgcagcaaaccaccatggcaaatgtattcctatgtaacaaacctgcacgttcagcaagtgtatcccagcactgaaagtaaaaaaaaaaaaaaaaaaaaaaaaaaaaaaaaa

SEQ ID NO: 104 - protein, 86 aa, Homo sapiens [sequence omitted]

SEQ ID NO: 105 - DNA, 479 bp, Homo sapiens
ctggcttcttcggagctgtgtaktcttaatttgctttgctgctggctggctgacagctgatgggggactcctcaggacggactcccttccagatgcacccatctccatccttctcaactccccaacctttgtcctccccactcttcgctcgcgcggcggtctgagaccaccaggaccagtttcaggggtttccttctccagcgagacttggcagaacaggctttaaaagcaaaggaggcagcggaagactgtgaattcctttggacaattgatgatatttatcattgtgcccagtttctacaaataaaagatgggtggattattttctcgatggaggacaaaaccttcaactgtagaagttctagaaagtatagataaggaaattcaagcattggaagaatttagggaaaaaaaaaaaaaaaaaaaaaaaaaaaaaaaaaaaaaaaaaaaaaaaaaaaaaaaaaaaaaaaaaaaaaaaa

SEQ ID NO: 106 - protein, 33 aa, Homo sapiens
MGGLFSRWRTKPSTVEVLESIDKEIQALEEFRE

SEQ ID NO: 107 - DNA, 333 bp, Homo sapiens
  unsure (70)
  unsure (156)
  unsure (184)
  unsure (207)
  unsure (308)
ctgacttactaatactaaagaagtggggggagctcgagagccagacggccagacaggcagacccctccanaggcccgccaggtgggcatggtcccccattttctttaaggcagcacctggagtggagagaggccactccctctccagcccccgatntggacccgggggaggggaggctgatgcntttggccccggcctggccaaaanagcccatccccagggcagtttcaggtgccggctgggccctgaatgtcaaggataatatatagcccgctcctgggtcctggagctgtggccctttgtactcntgttgtgtccattgtgtgtgtgcgt

SEQ ID NO: 108 - DNA, 611 bp, Homo sapiens
  unsure (62)
  unsure (185)
  unsure (192)
  unsure (249)
  unsure (290)
gtccattgtgtgtgtgcgtggggacagaggcctggaaatgcggaggactatacagagaaggnaggttttgtgaaggccaggcagggttggaggccgggggtgtgagaggagaggcccatagggctgagtggggtcgggtgaggcagaggtcagaaacagaagagctgcagttgctggagctgggntgagaantgggctgcctcctgccatccccccgtctcctccccttctccccttggtgcccccctntgctcagaatctgaagtagttccctcctcagcaatttcatntcttgaacactgactcacaccttttaggcacctactgtgtgcatagcattccaccaggactcatctcccttccttctcagggggtcccgagccccgactagctttgccctaactccttcatcaaaagaccccccgccagcttcccacacctcatacgcagccacatctgccctattctccatgctttccagcttgcctgcccttcctcatctctccctgcctgtgcagacctccacccttctttcctccacccctccatcccccaatgcttgtagaccttccattcattccgtctcatcgtgcgtggtctctgatcgtcca

SEQ ID NO: 109 - protein, 47 aa, Homo sapiens
MLSSLPALPHLSLPVQTSTLLSSTPPSPNACRPSIHSVSSCVVSDRP

SEQ ID NO: 110 - DNA, 274 bp, Homo sapiens
atccagggcgtggggagaccattggcatttgggaaccattttccttcgaacggcttccccttgagctgagcattctgcttgctgcagtagacgggtcgccttttgcccataccgaaattttctgaaattaaatcgcacacccccaccatttcctctcccctgggatctggaggaacatcatacatagtaggtgaatcgttttgtagagtgaagaatgctaatgtaaagcaaatagtcacccacgttcccttgtaaatccaaaaaaaaaaaaaaa

SEQ ID NO: 111 - DNA, 1646 bp, Homo sapiens
catcgggtggactagctgggatctccgcattggatttggggctgattaccactgcttgcctattattattgttgttgttactactattatttttttttacccaagggagaaagacaaaaaaacggtgggatttatttaacatgatcttggcaaacgtcttctgcctcttcttctttctagacgagaccctccgctctttggccagcccttcctccctgcagggccccgagctccacggctggcgccccccagtggactgtgtccgggccaatgagctgtgtgccgccgaatccaactgcagctctcgctaccgcactctgcggcagtgcctggcaggccgcgaccgcaacaccatgctggccaacaaggagtgccaggcggccttggaggtcttgcaggagagcccgctgtacgactgccgctgcaagcggggcatgaagaaggagctgcagtgtctgcagatctactggagcatccacctggggctgaccgagggtgaggagttctacgaagcctccccctatgagccggtgacctcccgcctctcggacatcttcaggcttgcttcaatcttctcagggacaggggcagacccggtggtcagcgccaagagcaaccattgcctggatgctgccaaggcctgcaacctgaatgacaactgcaagaagctgcgctcctcctacatctccatctgcaaccgcgagatctcgcccaccgagcgctgcaaccgccgcaagtgccacaaggccctgcgccagttcttcgaccgggtgcccagcgagtacacctaccgcatgctcttctgctcctgccaagaccaggcgtgcgctgagcgccgccggcaaaccatcctgcccagctgctcctatgaggacaaggagaagcccaactgcctggacctgcgtggcgtgtgccggactgaccacctgtgtcggtcccggctggccgacttccatgccaattgtcgagcctcctaccagacggtcaccagctgccctgcggacaattaccaggcgtgtctgggctcttatgctggcatgattgggtttgacatgacacctaactatgtggactccagccccactggcatcgtggtgtccccctggtgcagctgtcgtggcagcgggaacatggaggaggagtgtgagaagttcctcagggacttcaccgagaacccatgcctccggaacgccatccaggcctttggcaacggcacggacgtgaacgtgtccccaaaaggcccctcgttccaggccacccaggcccctcgggtggagaagacgccttctttgccagatgacctcagtgacagtaccagcttggggaccagtgtcatcaccacctgcacctctgtccaggagcaggggctgaaggccaacaactccaaagagttaagcatgtgcttcacagagctcacgacaaatatcatcccagggagtaacaaggtgatcaaacctaactcaggccccagcagagccagaccgtcggctgccttgaccgtgctgtctgtcctgatgctgaaacaggccttgtaggctgtgggaaccgagtcagaagatttttgaaagctacgcagacaagaacagccgcctgacgaaatggaaacacacacagacacacacacaccttgcaaaaaaaaaaaaaaa

SEQ ID NO: 112 - protein, 464 aa, Homo sapiens
MILANVFCLFFFLDETLRSLASPSSLQGPELHGWRPPVDCVRANELCAAESNCSSRYRTLRQCLAGRDRNTMLANKECQAALEVLQESPLYDCRCKRGMKKELQCLQIYWSIHLGLTEGEEFYEASPYEPVTSRLSDIFRLASIFSGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKQAL

SEQ ID NO: 113 - DNA, 355 bp, Homo sapiens
  unsure (133)
  unsure (151)
  unsure (196)
  unsure (228)
ctctcatttttcctttcgaagtgatatccactcgaacgagatcaaatctgtaagctggagatacaacttccactacataagatccagaaggtatatcatgaaccacaaaactcccatctgtcttaaggaaacngacgtgctcttctccgtntaccagcactcgggccgccgagatccagtcctgaggcttcacccntggaacaactgcacgcccctcaatcttgaagngatctcctatgccgaccccactccctcccgatccctcagcagcagccccgggcacctccgagttctggacatcccccgatagcagcagcagcagcaggacgggaaagaagccccacagagcggccgc

SEQ ID NO: 114 - DNA, 587 bp, Homo sapiens
ggctggacagactttctaatgaacccaatggttatgatgatggttcttcctttattgatatttgtgcttctgcctaaagtggtcaacacaagtgatcctgacatgagacgggaaatggagcagtcaatgaatatgctgaattccaaccatgagttgcctgatgtttctgagttcatgacaagactcttctcttcaaaatcatctggcaaatctagcagcggcagcagtaaaacaggcaaaagtggggctggcaaaaggaggtagtcaggccgtccaragctggcatttgcacaaacacggcaacactgggtggcatccaagtcttggaaaaccgtgtgaagcaactactataaacttgagtcatcccgacgttgatctcttacaactgtgtatgttaactttttagcacatgttttgtacttggtacacgagaaaacccagctttcatcttttgtctgtatgaggtcaatattgatgtcactgaattaattacagtgtcctatagaaaatgccattaataaattatatgaactactatacattatgtatattaattaaaacatcttaatccagaaaaaaaaaaaaaa

SEQ ID NO: 115 - protein, 81 aa, Homo sapiens
MNPMVMMMVLPLLIFVLLPKVVNTSDPDMRREMEQSMNMLNSNHELPDVSEFMTRLFSSKSSGKSSSGSSKTGKSGAGKRR

SEQ ID NO: 116 - DNA, 601 bp, Homo sapiens
gatgtacctaggcaccgcgcagcctgcggcccccaacaccacctcccccgagctcaacctgtcccacccgctcctgggcaccgccctggccaatgggacaggtgagctctcggagcaccagcagtacgtgatcggcctgttcctctcgtgcctctacaccatcttcctcttccccatcggctttgtgggcaacatcctgatcctggtggtgaacatcagcttccgcgagaagatgaccatccccgacctgtacttcatcaacctggcggtggcggacctcatcctggtggccgactccctcattgaggtgttcaacctgcacgagcggtactacgacatcgccgtcctgtgcaccttcatgtcgctcttcctgcaggtcaacatgtacagcagcgtcttcttcctcacctggatgagcttcgaccgctacatcgccctggccagggccatgcgctgcagcctgttccgcaccaagcaccacgcccggctgagctgtggcctcatctggatggcatccgtgtcagccacgctggtgcccttcaccgccgtgcacctgcagcacaccgacgaggcctgcttctgtttcgcggatgtccgggag

SEQ ID NO: 117 - protein, 200 aa, Homo sapiens
MYLGTAQPAAPNTTSPELNLSHPLLGTALANGTGELSEHQQYVIGLFLSCLYTIFLFPIGFVGNILILVVNISFREKMTIPDLYFINLAVADLILVADSLIEVFNLHERYYDIAVLCTFMSLFLQVNMYSSVFFLTWMSFDRYIALARAMRCSLFRTKHHARLSCGLIWMASVSATLVPFTAVHLQHTDEACFCFADVRE

SEQ ID NO: 118 - DNA, 419 bp, Homo sapiens
  unsure (80)
  unsure (178)
acgcctgagcgtcctccatcttccaggatggcagcaatggcgctgtgcggcctcaccaggcccacgaggagcagcagcgntcggcccggagcagcaggaaggcccttttgtggagcgcccgccgtctgctccggggtggttcagtcactgcttgttgacatcaacatggcaattgcantcatgtggactgggaccgtgcgagctgccgtgtgggttagtcgggtgccaggacaatgaaatactccagcacgtgtggctgacgaatttgtttttacagaaataacagctggggacaactgcggtgatgatgtaaaaaccttcccataaaatgtaagaaaagctgatgaggctggtgacgttcagcctttgtcaataaacctgtcatgtgcggaaaaaaaaaaaaaaaaaaaaaaaaaaaa

SEQ ID NO: 119 - DNA, 714 bp, Homo sapiens
  unsure (646)
  unsure (649)
agccattgggacaggaaatgccaaacaacacccagataaggttgctgaagccataattgatgccattgaagactttgtccagaaaggatcagcccagtctgtgaaaaaagttaaagttgttatctttctgcctcaagtactggatgtgttttatgccaacatgaagaaaagagaagggactcagctttcttcccaacagyctartswtsyytwwmyttkyakcatttttgggcttttcaaagcaatctccccaaaaaaagaatcatttggttttggaaaagaaaacagaatcagcaacttttcgggtgtgtggtgaaaatgtcacgtgtgtggaatacgctatctcctggctacaagacctgattgaaaaagaacagtgtccttacaccagtgaagatgagtgcatcaaagactttgatgaaaaggagtatcaggagttgaatgagctgcagaagaagttaaatattaacatttccctggaccataagagacctttgattaaggttttgggaattagcagagatgtgatgcaggctagagatgaaattgaggcgatgatcaagagagttcgattggccaaagaacaggaatcccgggcagattgtatcagtgagtttatagaatggcagtataatgacaataacanttntcattgttttaacaaaatgaccaatctgaaattagaggatgcaaggagagaaaaaaaaaaaaaaa

SEQ ID NO: 120 - protein, 159 aa, Homo sapiens
  UNSURE (141)..(142)
FLGFSKQSPQKKNHLVLEKKTESATFRVCGENVTCVEYAISWLQDLIEKEQCPYTSEDECIKDFDEKEYQELNELQKKLNINISLDHKRPLIKVLGISRDVMQARDEIEAMIKRVRLAKEQESRADCISEFIEWQYNDNNXXHCFNKMTNLKLEDARRE

SEQ ID NO: 121 - DNA, 2681 bp, Homo sapiens
  unsure (2656)
cataacttccaatcttcttggcctggcactaaggacctactacagactggcactgaaatatcttccctaccttatcattccttgtgcttctccacaagcccactcttccccttcatcatacatgtgccgacctttcctgtctcttttactttgcagcaccaaatgctttctactttgtggtctaggaggaacacatgtcacttttgtaagctgctcgaaagcaggggccacaccttcatccttgttttccacacaacaccaagcacttagtagacacccaataaatcattgctgaatgaatgtattcagcctggaattgcactaggattttttggccaacacattgtattcttaactgataccagacttccaatcaaataaaatccttaagcctttttcatagtctttaattaaactacttctcttccattatttccctttgcctacttttgaactgatattcagaacttttctgttaatgtttaattttcatccattattcttgtctgtacagatctttttgatttttgactctcttatctagttttttttttttttttttggcttcccgttgtgttatccacaggcacaatgggtatattgactatcgttcattgaagttgttgatacaaatgttgaacaggagaaaaaccagtagcttgccaacttggcacctcattctctagcttgacagtaatctcttatctagtagtttttagatatggttatttaatcaaatatcactagcttctaattttgttataattcattcatgtatacagacaatgaagaatcatctttcttcattcaacacatttattgggaaccaatcattgtccaaggcaatatgctaggcattgtgtaatagaaagcgattaagcttttacccctgtcctctttggcctgcaaagagcaggtaagattcatgcacagacaaatgtagtaccaggtaaatagtgaggccaaacacaagaggcaggtcaaagggctttggattcagaggatggagaagctctttctaggctggtgaaatatttgcagaatgagtaggctcataagtggaggtgaggtacaggctaagctgctgtaagaaagagtccaaaatgcaaaggatttaccacaatgcagttaatttttctcacaacggtcgaggtaggcaggtggtccaagtccagttaatcagccctcctcaacacaaggcttccccttgtaagctcaggatgaccgctccagttctcatcatctcccaggcagaaaaacgggagaaagagaactagacgtaccacccagtcattttaaggacatagccaggccaggtgcagtgactcacatctgtaatccaagcactttgggaggtcggaggctggaggatagcttgaagccaggagtttgagaccagcttggataagaaagcaagaccttgtctctacaaaaaatttaaaaattaaccatgtgtggtggtgtgcacctatagtcccagctactcaggaggctgaggtggaaggatcacttgagcacaggagttggaggatacagtgagccatgatcacaccacctcactccagcctgggcaacatagtgagaccccatctctttaaaaaacaaaacaaaaaaacatcaccaggaagttgcttacatgtcttctattcctataacatcggcctgagcttagtcacatggccacacccagcagaaagaactctaggaaatatggtcttatgctaggtaaccccaaacccagctaaaactgttgcttttgaagaagggtgaaacagacaatgtggaggagaattaccagtctgccacaaagagaaagaattctatgtgaggaaaacgctctagaagagggagctgattaattagttatatctcagccgagaggatgttatgagaaacacagatttgggtttaatagaaaaacttagaacccttcaagaaggaatgggcggacttgagagtgtcctccgcttctgaaaatgatcaagaaaaatggtaagaatgtacacctcaagggtggatggtaaaggtcagcctttaagcctcaatggcggtgtggttgtaggtggggtgggggtggaggccccgaagacaggcagactaccacagtagccccgatggaagcagtgagaatgaactggaaggagcggctgtgggagcgacaacgtgatgagaataaacccggcttggctctgccctgtgcacacacaggtgagctgtgtgctcccggatgtgtcagctggtatatgcgtctgtcagaaggcagctggggagcactgttagctcagagactaagaggcagacccaggaagcccttctttgcattggtcagggtttgctgcattttcccaagcccaggaaatggaacccagttcttcttctttctgtgtaaaataataagcatcacaataggctgtgctcatgagaacgcattttgtttccacagaaatgttttttctcactctgtcctgattttgatttctgttaaactcagtaaacacattaccaaattttaaaataaggtgacttgttttccccaactcacagttcaccaaaggtatttcatctgtttgttctgaaaatgcagctgctgtctagatttatgtgtgctctgacaagaaatgttttgtgtaacaataaaaatcatttcctttgatgnaaaaaaaaaaaaaaaaaaaaaaaaa

SEQ ID NO: 122 - protein, 132 aa, Homo sapiens
MEAVRMNWKERLWERQRDENKPGLALPCAHTGELCAPGCVSWYMRLSEGSWGALLAQRLRGRPRKPFFALVRVCCIFPSPGNGTQFFFFLCKIISITIGCAHENAFCFHRNVFSHSVLILISVKLSKHITKF

SEQ ID NO: 123 - DNA, 1585 bp, Homo sapiens
ctaagctatttgattctaggtctagaatgttatctcttattagaggatatgttaattttcctgcattttattcatttattaacttaacatctctgattgcctaccatgtgtcaggctctgtactaaggattgaggacccaaagatgaacaaaacatggggcctaattcaaagatttcacaatctggagagaaagtcagccacatacaaaaaattataaggtagaatgtgctataaaaaatgatttaggtacagtggaagtttagaaaggcttcactaaagatgtcgtatttgaattgggtctgagcatgaattagtcttcaggtgagggagggggttaaagaaaactctaatgaaggaactctagtatgtgtgaaggcaaggaggctggtgtgtttaggttgcagcaaaccaagtacaaccaggcttggacttgagtgactggaaagacaggaagatgccatgctgagaaaaactgcccatgccaagctgagaaatgttcagcagaaacataaggtgagctgcatatgtcatttaaaatgttctaggagccacatttttaaaaaatcaaaattaacaagtcaaaaaataaaaagcaatggggggagattaaatgcatattactaagtgaaagaagccaatccaaaaaggctacatacctgtatgattccaactatatgacattctgaaaaaggccaaactatagagacagtaaatgatcagtggttgccaggagttagggaggagggagggatgaacaggcagagcacagaagatttttagagcagtagaactattttgtatgatattataatattgtagatacatatcattataaatttgtccaaacccatagaatgtacaccaagagtgaactctaatgtaaactgtggactttgggtgataatgatgtgtcagtgtagattgatcagttgtaacaaatgtaccattctggtggggaatgttgataatggagtaggctatgcatgtgtggggcagtgggtatatgggaaatatctataccttctggtcagttttgctgtgaacttgatctaaaaaatagcctactaagaaacacaagtcaaattaattttaataatacattttatttaacccaatttatcagaaatactaatattttaacatgtaattgatataaaagttattaactagatattttactttttttggtactgagtctttgaaatctggtctgtattttacatttacagtacatctcaattcacattagccacatttcagatactcagtacatacatgagtacctatggctagtggctgctgtgttggacagggcaggtcttgaaacctggacttgcctgactcagaagcctcaattctcagccacagtgatatcctgctccctaagtactataatgataaacacaagaggagaggagctttcagatgatcatctaatcccatgacgttagctgttgctctccacactgcccggtggctccagtctgaagcatctaggcagtgctgtccaacagaaatacaatgagagccaatacgtgataagtgtcctatgggccacattgaaacagtaaaaaaaaaaaaaaa

SEQ ID NO: 124 - protein, 63 aa, Homo sapiens [sequence omitted]

SEQ ID NO: 125 - DNA, 625 bp, Homo sapiens
gatcccaccagttctgcctggcttcctccatccccagaggcactaaaagcagtattttaaggttggtgtcttactccctggaagcctgaaatgggtggaatagcggtaaggcttgagtaaaactaggggacagaggttcttatttgtcgattttattttataatttgaccacagcatctgaactccctctctccctggaataagtatttttcccacatttttggatatatgtatggtagacaatttttttttaagacacagagataaatgttttcctgctttggttacctttcctttcccctttaaaaggaattagctatagaactgctttgtaaagatgcttcttgatattttacttttgttccttttccctaatcattcccttttctccccactcctccagaaggcataacccttctctccacaccccctacccccacccccgtcctaggctcccatcctttccatcaagaccttcattagcttatgatatttgctgccgagatgttataacaaggactcgttcatgtatataagctatttcttgatccatttaaaaggaattgtacattgtgtaggaaaaaaaaaaaaaaaaaaaaaaaaaaaaaaaaaaaaaaaaaaaaaa

SEQ ID NO: 126 - protein, 24 aa, Homo sapiens
MLLDILLLFLFPNHSLFSPLLQKA

SEQ ID NO: 127 - DNA, 1946 bp, Homo sapiens
gaagctggggcccagcggaggtagcagcagacgcctgagagcgaggccgaggcccctcagggtttggagaccctgacacacccaccttctcacctgggctctgcgtatcccccagccttgagggaagatgaagcctaaactgatgtaccaggagctgaaggtgcctgcagaggagcccgccaatgagctgcccatgaatgagattgaggcgtggaaggctgcggaaaagaaagcccgctgggtcctgctggtcctcattctggcggttgtgggcttcggagccctgatgactcagctgtttctatgggaatacggcgacttgcatctctttgggcccaaccagcgcccagccccctgctatgacccttgcgaarcagtgctggtggaaagcattcctgarggcctggacttccccaatgcctccacggggaacccttccaccagccaggcctggctgggcctgctcgccggtgcgcacagcagcctggacatcgcctccttctactggaccctcaccaacaatgacacccacacgcaggagccctctgcccagcagggtgaggaggtcctccggcagctgcagaccctggcaccaaagggcgtgaacgtccgcatcgctgtgagcaagcccagcgggccccagccacaggcggacctgcaggctctgctgcagagcggtgcccaggtccgcatggtggacatgcagaagctgacccatggcgtcctgcataccaagttctgggtggtggaccagacccacttctacctgggcagtgccaacatggactggcgttcactgacccaggtcaaggagctgggcgtggtcatgtacaactgcagctgcctggctcgagacctgaccaagatctttgaggcctactggttcctgggccaggcaggcagctccatcccatcaacttggccccggttctatgacacccgctacaaccaagagacaccaatggagatctgcctcaatggaacccctgctctggcctacctggcgagtgcgcccccacccctgtgtccaagtggccgcactccagacctgaaggctctactcaacgtggtggacaatgcccggagtttcatctacgtcgctgtcatgaactacctgcccactctggagttctcccaccctcacaggttctggcctgccattgacgatgggctgcggcgggccacctacgagcgtggcgtcaaggtgcgcctgctcatcagctgctggggacactcggagccatccatgcgggccttcctgctctctctggctgccctgcgtgacaaccatacccactctgacatccaggtgaaactctttgtggtccccgcggatgaggcccaggctcgaatcccatatgcccgtgtcaaccacaacaagtacatggtgactgaacgcgccacctacatcggaacctccaactggtctggcaactacttcacggagacggcgggcacctcgctgctggtgacgcagaatgggaggggcggcctgcggagccagctggaggccattttcctgagggactgggactccccttacagccatgaccttgacacytcagytgacagsgtgggcaacgcctgccgcytgytctgaggcccgatccagtgggcaggccaaggcctgctgggcccccgcggacccaggtgytctgggtcacggtccctgtccccgcacccccgyttytgtytgccccattgtggctcctcaggytytytcccctgytctcccacctytacctccacccccaccggcctgacgctgtggccccgggacccagcagagctgggggagggatcagcccccaaagaaatgggggtgcatgctgggcctggccccctggcccacccccayttttcagggcaaaaagggcccagggttataataagtaaataacttgtctgtaaaaaaaaaaaaaaaaaaaaaaaaaaaaaaaaaaaaa

SEQ ID NO: 128 - protein, 490 aa, Homo sapiens
  UNSURE (83)
  UNSURE (480)
  UNSURE (482)
  UNSURE (490)
MKPKLMYQELKVPAEEPANELPMNEIEAWKAAEKKARWVLLVLILAVVGFGALMTQLFLWEYGDLHLFGPNQRPAPCYDPCEXVLVESIPEGLDFPNASTGNPSTSQAWLGLLAGAHSSLDIASFYWTLTNNDTHTQEPSAQQGEEVLRQLQTLAPKGVNVRIAVSKPSGPQPQADLQALLQSGAQVRMVDMQKLTHGVLHTKFWVVDQTHFYLGSANMDWRSLTQVKELGVVMYNCSCLARDLTKIFEAYWFLGQAGSSIPSTWPRFYDTRYNQETPMEICLNGTPALAYLASAPPPLCPSGRTPDLKALLNVVDNARSFIYVAVMNYLPTLEFSHPHRFWPAIDDGLRRATYERGVKVRLLISCWGHSEPSMRAFLLSLAALRDNHTHSDIQVKLFVVPADEAQARIPYARVNHNKYMVTERATYIGTSNWSGNYFTETAGTSLLVTQNGRGGLRSQLEAIFLRDWDSPYSHDLDTSXDXVGNACRLX

SEQ ID NO: 129 - DNA, 6254 bp, Homo sapiens
agggtcctgggcagtcgcgccagagctgagcggagggcgcggcgcgagaacgaatctttgtgacattctctctcagcattctttatcccctgtttgctgaagacttcgaccaaagctggtcttagctgttggcattctcctgagaaaaggatagcttcagaaatcagaaaaacatttgggaggtgtctagcccagtggaccttctgaagagcaatgctaagaagacgtttggtttaaagaattaaaaggaagaacaacttaagagcttcttcaaagttccccgcatgaaaattacttaaacgttgcacacaacgtttcacaaaatcttttgtgaaagaagaaaaggaaattcagtgtgtgagtctcagcaggagttaagctaatgcagcttaacatcatgccgacaaagaagcgcttatctgcgggcagagtgcccctgattctcttcctgtgccagatgattagtgcactggaagtacctcttgatccaaaacttcttgaagacttggtacagcctccaaccatcacccaacagtctccaaaagattacattattgaccctcgggagaatattgtaatccagtgtgaagccaaagggaaaccgcccccaagcttttcctggacccgtaatgggactcattttgacatcgataaagaccctctggtcaccatgaagcctggcacaggaacgctcataattaacatcatgagcgaagggaaagctgagacctatgaaggagtctatcagtgtacagcaaggaacgaacgcggagctgcagtttctaataacattgttgtccgcccatccagatcaccattgtggaccaaagaaaaacttgaaccaatcacacttcaaagtggtcagtctttagtacttccctgcagacccccaattggattaccaccacctataatattttggatggataattcctttcaaagacttccacaaagtgagagagtttctcaaggtttgaatggggacctttatttttccaatgtcctcccagaggacacccgcgaagactatatctgttatgctagatttaatcatactcaaaccatacagcagaagcaacctatttctgtgaaggtgatttcagctaaatcaagtagagagaggccaccaacatttttaactccagaaggcaatgcaagtaacaaagaggaattaagaggaaatgtgctttcactggagtgcattgcagaaggactgcctaccccaattatttactgggcaaaggaagatggaatgctacccaaaaacaggacagtttataagaactttgagaaaaccttgcagatcattcatgtttcagaagcagactctggaaattaccaatgtatagcaaaaaatgcattaggagccatccaccataccatttctgttagagttaaagcggctccatactggatcacagcccctcaaaatcttgtgctgtccccaggagaggatgggaccttgatctgcagagctaatggcaaccccaaacccagaattagctggttaacaaatggagtcccaatagaaattgcccctgatgaccccagcagaaaaatagatggcgataccattattttttcaaatgttcaagaaagatcaagtgcagtatatcagtgcaatgcctctaatgaatatggatatttactggcaaacgcatttgtaaatgtgctggctgagccaccacgaatcctcacacctgcaaacacactctaccaggtcattgcaaacaggcctgctttactagactgtgccttctttgggtctcctctcccaaccatcgagtggtttaaaggagctaaaggaagtgctcttcatgaagatatttatgttttacatgaaaatggaactttggaaattcctgtggcccaaaaggacagtacaggaacttatacgtgtgttgcaaggaataaattagggatggcaaagaatgaagttcacttagaaatcaaagatgctacatggatcgttaaacagcccgaatatgcagttgtgcaaagagggagcatggtgtcctttgaatgcaaagtgaaacatgatcacaccttatccctcactgtcctgtggctgaaggacaacagggaactgcccagtgatgaaaggttcactgttgacaaggatcatctagtggtagctgatgtcagtgacgatgacagcgggacctacacgtgtgtggccaacaccactctggacagcgtctccgccagcgctgtgcttagcgttgttgctcctactccaactccagctcccgtttacgatgtcccaaatcctccctttgacttagaactgacagatcaacttgacaaaagtgttcagctgtcatggaccccaggcgatgacaacaatagccccattacaaaattcatcatcgaatatgaagatgcaatgcacaagccagggctgtggcaccaccaaactgaagtttctggaacacagaccacagcccagctgaagctgtctccttacgtgaactactccttccgcgtgatggcagtgaacagcattgggaagagcttgcccagcgaggcctctgagcagtatttgacgaaagcctcagaaccagataaaaaccccacagctgtggaaggactgggatcagagcctgataatttggtgattacgtggaagcccttgaatggtttcgaatctaatgggccaggccttcagtacaaagttagctggcgccagaaagatggtgatgatgaatggacatctgtggttgtggcaaatgtatccaaatatattgtctcaggcacgccaacctttgttccatacctgatcaaagttcaggccctgaatgacatggggtttgcccccgagccagctgtagtcatgggacattctggagaagacctcccaatggtggctcctgggaacgtgcgtgtgaatgtggtgaacagtaccttagccgaggtgcactgggacccagtacctctgaaaagcatccgaggacacctacaaggctatcggatttactattggaagacccagagttcatctaaaagaaacagacgtcacattgagaaaaagatcctcaccttccaaggcagcaagactcatggcatgttgccggggctagagccctttagccactacacactgaatgtccgagtggtcaatgggaaaggggagggcccagccagccctgacagagtctttaatactccagaaggagtccccagtgctccctcgtctttgaagattgtgaatccaacactggactctctcactttggaatgggatccaccgagccacccgaatggcattttgacagagtacaccttaaagtatcagccaattaacagcacacatgaattaggccctctggtagatttgaaaattcctgccaacaagacacggtggactttaaaaaatttaaatttcagcactcgatataagttttatttctatgcacaaacatcagcaggatcaggaagtcaaattacagaggaagcagtaacaactgtggatgaagctggtattcttccacctgatgtaggtgcaggcaaagcgatggcaagccggcaggtggatattgcaactcagggctggttcattggtctgatgtgtgctgttgctctccttatcttaattttgctgattgtttgcttcatcagaagaaacaagggtggtaaatatccagttaaagaaaaggaagatgcccatgctgaccctgaaatccagcctatgaaggaagatgatgggacatttggagaatacagtgatgcagaagaccacaagcctttgaaaaaaggaagtcgaactccttcagacaggactgtgaaaaaagaagatagtgacgacagcctagttgactatggagaaggggttaatggccagttcaatgaggatggctcctttattggacaatacagtggtaagaaagagaaagagccggctgaaggaaacgaaagctcagaggcaccttctcctgtcaacgccatgaattcctttgtttaatttttaagctctttgccaatattccatttctctagaatgtttatcctaagcacttgtttgtcagccctctcatactatgaacatatgggtagagagtatattttctgctgtatgttagtattatgagaatagttacagcaaaaacataactcagtcaaatgatatgttaatatgaactggaatgcaaagtgcatactttttcattcaaaatgggtattcttgatttcctcagaactgataaaaaataatgcaacatcaccaacagatcctgttatttcctctgcaggatacagttcaatatgatgcatgaaaaatgctccacatttaaaggacatacccgtgtatgttatgaaaacatggtttgatactttgtttatactaccctcagctgaacccctatatatgaattccgttttcattgtcaagaatgttactgtagtattctctagaacttcaatgtctttgtggacattgttgtgaaattggtgactatgtatagctgtcgttagtctttttgggagactgttaggaacagtttgtacagtatatacttgctaaatgagttcattatgacagtcacattgctgatgcttactgagaactattacctactcttggctcctgttactccgtaggcttcttaatcttccaggcattacagcagcacagtgttctactttttacatcatttctatgttcggttgtttttaggcataaacaatgtgtattgcagtgcatttcggcatttgtgccatactgaaagaatcaaaaacaaatcatccaaattaaatttcaaacattatttcagagaacacagggcaagacacatacagtgccttcagatattaagcattccacaacatcgtgcattctgtatcagctggtccagtccattctgggtcctagattactgtcattgtctaaaagtaacttttaaaaagcagagttcatgaaaactgcaatgctgggaaaagaaggaaacatgaaaataaaaataagacagtttattagaaatagcatttcctcataagcataaaaagaaatctttgttgccaactgaagcacatgggattttgtggtcctttatggtttctataacattcagtaagaaagatgtcaacatgctagaaaattaattttaaaactaagttattccaacactaaaagcatacaacagcatgccaacagtaatatattattctccaagactttacctatgtaagtgttcaaaactctgcagcattaaacaacgtgtatgcaaattgttatggatacatttcagaatctaagaaatcaggcaagtgcttaaaaggccaacggtccaagggattacatctgcagtttaaaaagtaaatatatattctatcgtattcataaacaatatctatcaaatgggttacctccaaatatgaaaatctataacaacctatggttgaaggaatgctcagtttcatttgccaataaattggtttctcataacttgcatcaagtttaattttaagtaaagctttttatatgtagatattttgttgaatttgtaaatacacttaaaatgtagatgctatatgcttataggtgttacatacaaataaacatgcaatgtttatgttgtactgtataagaggtaagctaattaatgcagtgaatgggattggaaagcatctacttaaatatctattgggttcccccctccccccaccttttttgctgtgaaactgaaatagtgaacttttctacgtattgacagcagatttttcgatgaaatcttcagagctttgcctatggggcacagtaggcctagtaacctggcatgtttgatatatgtaggtaaagcataatttaaagtaatcccaggtaaagatggccctaaatactttcatgtctctatattcatttttcacagatccacctgtctcttgaaaatataaaaagacaaaacaggtttgccttggcatcagagagcacaaagattaaaagttactttaaatttgccaatattttgggagaacaataaaactacattttttcytyttccatactggtagatgcgaaatttatctgtgcatgaaagggtcacttctgtaatagtgcacagatttggtattaaaaattaaatgtggttttaaaagttcctytytcttttgtaatttatgttcccaattgagtgtgaatgtccaagtaatggtgtatgtaatggtacaggcaaatgtgactggatttccctcaaaaaagtaactattaaacagtyttgatctcaaaaaaaaaaaaaaaaaaaaaaaaaaaaaaaaaaaaa

SEQ ID NO: 130 - protein, 1192 aa, Homo sapiens
MQLNIMPTKKRLSAGRVPLILFLCQMISALEVPLDPKLLEDLVQPPTITQQSPKDYIIDPRENIVIQCEAKGKPPPSFSWTRNGTHFDIDKDPLVTMKPGTGTLIINIMSEGKAETYEGVYQCTARNERGAAVSNNIVVRPSRSPLWTKEKLEPITLQSGQSLVLPCRPPIGLPPPIIFWMDNSFQRLPQSERVSQGLNGDLYFSNVLPEDTREDYICYARFNHTQTIQQKQPISVKVISAKSSRERPPTFLTPEGNASNKEELRGNVLSLECIAEGLPTPIIYWAKEDGMLPKNRTVYKNFEKTLQIIHVSEADSGNYQCIAKNALGAIHHTISVRVKAAPYWITAPQNLVLSPGEDGTLICRANGNPKPRISWLTNGVPIEIAPDDPSRKIDGDTIIFSNVQERSSAVYQCNASNEYGYLLANAFVNVLAEPPRILTPANTLYQVIANRPALLDCAFFGSPLPTIEWFKGAKGSALHEDIYVLHENGTLEIPVAQKDSTGTYTCVARNKLGMAKNEVHLEIKDATWIVKQPEYAVVQRGSMVSFECKVKHDHTLSLTVLWLKDNRELPSDERFTVDKDHLVVADVSDDDSGTYTCVANTTLDSVSASAVLSVVAPTPTPAPVYDVPNPPFDLELTDQLDKSVQLSWTPGDDNNSPITKFIIEYEDAMHKPGLWHHQTEVSGTQTTAQLKLSPYVNYSFRVMAVNSIGKSLPSEASEQYLTKASEPDKNPTAVEGLGSEPDNLVITWKPLNGFESNGPGLQYKVSWRQKDGDDEWTSVVVANVSKYIVSGTPTFVPYLIKVQALNDMGFAPEPAVVMGHSGEDLPMVAPGNVRVNVVNSTLAEVHWDPVPLKSIRGHLQGYRIYYWKTQSSSKRNRRHIEKKILTFQGSKTHGMLPGLEPFSHYTLNVRVVNGKGEGPASPDRVFNTPEGVPSAPSSLKIVNPTLDSLTLEWDPPSHPNGILTEYTLKYQPINSTHELGPLVDLKIPANKTRWTLKNLNFSTRYKFYFYAQTSAGSGSQITEEAVTTVDEAGILPPDVGAGKAMASRQVDIATQGWFIGLMCAVALLILILLIVCFIRRNKGGKYPVKEKEDAHADPEIQPMKEDDGTFGEYSDAEDHKPLKKGSRTPSDRTVKKEDSDDSLVDYGEGVNGQFNEDGSFIGQYSGKKEKEPAEGNESSEAPSPVNAMNSFV

SEQ ID NO: 131 - DNA, 4253 bp, Homo sapiens
gcgatcagaagactgaggcagtgctgcaggtgaacatggctgtagcctaccaatccttcattttggctgacttcataattgaacctgctcactgccacccagtcgttcacatggcaaaccccagtgttccttaagtgggctgaaaagaatggaatcctagacctgtcttgttagagtaraagctcattgagctgcctgcccttccatattgcttgtggacctatgaatggtgctctttaaaatgtcagtgtagagggactgggaatcctgtattcctgccctatcattctgtaactttgagaagcatgtgggctcagtgcaractacttatttttagttctgttagaacaggatttttaagaaaccatctgaacagaagaaataaagcggaaggtaagctcagctcatgtgcccatcaagctgcataaatctccatctgctctgggaaacattcatgggaacagaaggattcttcttcctgagaatgttgttttcattatataatcatagatacagcatgtgggtgatttctgtgtacaaatactttacccatgagaaatgtatagctgttagatatgtgtgggctcacacatatatgcaatagatgctatacatagcgatgtgcatgtgtatttgtatatatttatgttcatattttaacatatgaaacatgaaatagaagtataatattatttttgtatccaaatagttaacagagcagatgcccttagattctaatatgttgttcagtcctgaagatagccaagttaaatataaaaacaggagaagaggaaaaataaaaktccattgagaaggaatagagagaaagaacrggtcttgtgaatgagaakttcatttggatcaacacagcatttcctaraatgagtctgcactgtgctttttgcararacraatgggctccgtaggctctccacggcttttgatggcaratgakttgtctctgtttcaratgaktcaagctccagtcctggaaggcagatgtcctcctccgatggtgggccaccgggccagtcaracacagacagctcccgtggaggagagcgacttcgacaccatgccagacattgagagtgataaaaacatcatccggaccaagatgttcctttacctgtcagatttgtccaggaaggaccggagaattgtcagcaaaaaatataaaatttatttttggaacatcatcaccattgctgtgttttacgcgctgcccgtgatccagctggtcattacctatcagacagtggtaaatgtcactggcaaccaggacatctgttactacaacttcctctgtgctcaccccttgggcgtcctgagtgccttcaacaacattctcagcaatctgggccacgtgcttctgggcttcctcttcctgctgatagtcttgcgccgcgacatcctccatcggagagccctggaagccaaggacatctttgctgtggagtacgggattcccaaacactttggtctcttctacgctatgggcattgcattgatgatggaaggggtgctcagtgcttgctaccatgtctgccctaattattccaacttccaattcgacacctccttcatgtacatgatcgctggcctgtgcatgctgaagctctatcagacccgccacccagacatcaatgccagcgcctactctgcctatgcctcctttgctgtggtcatcatggtcaccgtccttggagtggtgtttggaaaaaatgacgtatggttctgggtcatcttctctgcaatccacgttctggcctcgctagccctcagcacccaaatatattatatgggtcgtttcaagatagatgtgtctgacacagatttgggaattttccggcgggctgccatggtgttctacacagactgtatccagcagtgtagccgacctctatatatggatagaatggtgttgctggttgtggggaatctggttaactggtccttcgccctctttggattgatataccgccccagggactttgcttcctacatgctgggcatcttcatctgtaaccttttgctgtacctggccttttacatcatcatgaagctccgcagctctgaaaaggtcctcccagtcccgctcttctgcatcgtggccaccgctgtgatgtgggctgccgccctatattttttcttccagaatctcagcagctgggagggaactccggccgaatcccgggagaagaaccgcgagtgcattctgctggatttcttcgatgaccatgacatctggcacttcctctctgctactgctctgtttttctcattcttggttttgttaactttggatgatgaccttgatgtggttcggagagaccagatccctgtcttctgaacctccaacattaagagaggggagggagcgatcaatcttggtgctgtttcacaaaaattacagtgaccacagcaaagtaaccactgccagatgctccactcaccctctgtagagccaactctgcattcacacaggaaggagaggggctgcgggagatttaaacctgcaagaaaggaggcagaaggggagccatgttttgaggacagacgcaaacctgaggagctgagaaacacttgctccttccatctgcagctttgggagtgcaacagggataggcactgcatccaagtcaactcaccatcttggggtccctcccaccctcacggagacttgccagcaatggcagaatgctgctgcacacttccctccagttgtcaccctgcccagaaaggccagcagcttggacttcctgcccagaaactgtgttggcccccttcacacctctgcaacacctgctgctccagcaagaggatgtgattctttagaatatggcggggaggtgaccccaggccctgccctactgggatagatgttttaatggcaccagctagtcacctcccagaagaaactctgtatatttcccccaggtttctgatgccatcagaagggctcaggagtggggtttgtcacacattcctcttaacaagtaactgtcactgggaccgagtcctgggtgcttacatattccttcgtgtcttcatctcactgacctgtgtggacctcatcactctgactctgccttcttggaaaggccctgtcactccacagatgtctggccagcttcaaggcagaaggaaaaacaggaaaagctcttttaacagcagcaggaacaagagaaatgactaaccatactaaaagactggtaacagcagcagcagccagacaggcctcaccttaaggacttgggctgccagagcaaattcagcagagcttatttggcctcccattcacacagctcagttctgtgcccacatcacctttggggaagaaatcagcattctaatcagggacactacttcaggagtcctccacagcgagtccgtcatctgtcactttatgtagatcagggttctagacttcttccctgaggttctcagaagcagctctcaggatgaacgtattgtcctcttcccctcttcttgcaaagtgcacagctaatctaatgttgtctctcggttgcacctgacattctctccccagtaaggtgttggcaagctcagcatctgggttccactctcacactgtctggcagctctgtgtctgagaagttctacattgaccaggcccccttgttgcctggagtatgacgtaatcagaaaatagacgtataaatgtgcacatgcgtatgtatttgcttgtgaaattaaagtcacctcttgcctctgctttcctgatcattcgttagagaaatggatcaggcatttttttaaattattattctttctctaaactatttgcattgtgttcaaaaacccattttagaagtttgaacagcaagcttttcctgattttaaaaacacaaagttgctttcaatgaaatattttgtgatttttttaaagtccccaaatgtgtacttagccttctgttattccttattctttaagcagtgttggcttccattgaccatatgaaggccaccaattaaatggttgtgttaatccaacatgtaaaaaactttttggcagggcacagtggctcacgcctgtaatcccaacactttgggaggctgaggcaggaggatcamttgagcccaggagattgacgccgcagtgaactatgattgtgcccctgcactccagcctggatgacagagtgagaccccatytyttaaaaaataaaaaaaaataaaaatttgaacctgtttcaaaaaaaaaaaaaaaaaaaaaaaaaaaaaaaaaa

SEQ ID NO: 132 - protein, 479 aa, Homo sapiens
  UNSURE (13)..(14)
  UNSURE (21)
MGSVGSPRLLMAXXLSLFQMXQAPVLEGRCPPPMVGHRASQTQTAPVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDVSDTDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 133 - DNA, 462 bp, Homo sapiens
gtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcargaaaattatggctcaagtatgggagactggggcttaccaaactcaaaggcc

SEQ ID NO: 134 - protein, 147 aa, Homo sapiens
MSQCTSSTIPSSSQEKDPKIKTETSEEGSGDLDNLDAILGDLTSSDFYNNSISSNGSHLGTKQQVFQGTNSLGLKSSQSVQSIRPPYNRAVSLDSPVSVGSSPPVKNISAFPMLPKQPMLGGNPRMMDSQENYGSSMGDWGLPNSKA

SEQ ID NO: 135 - DNA, 119 bp, Homo sapiens
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 136 - DNA, 3316 bp, Homo sapiens
ggtaagatggcggctgtgagtctgcggctcggcgacttggtgtgggggaaactcggccgatatcctccttggccaggaaagattgttaatccaccaaaggacttgaagaaacctcgcggaaagaaatgcttctttgtgaaattttttggaacagaagatcatgcctggatcaaagtggaacagctgaagccatatcatgctcataaagaggaaatgataaaaattaacaagggtaaacgattccagcaagcggtagatgctgtcgaagagttcctcaggagagccaaagggaaagaccagacgtcatcccacaattcttctgatgacaagaatcgacgtaattccagtgaggagagaagtaggccaaactcaggtgatgagaagcgcaaacttagcctgtctgaagggaaggtgaagaagaacatgggagaaggaaagaagagggtgtcttcaggctcttcagagagaggctccaaatcccctctgaaaagagcccaagagcaaagtccccggaagcggggtcggcccccaaaggatgagaaggatctcaccatcccggagtctagtaccgtgaaggggatgatggccggaccgatggccgcgtttaaatggcagccaaccgcaagcgagcctgttaaagatgcagatcctcatttccatcatttcctgctaagccaaacagagaagccagctgtctgttaccaggcaatcacgaagaagttgaaaatatgtgaagaggaaactggctccacctccatccaggcagctgacagcacagccgtgaatggcagcatcacacccacagacaaaaagataggatttttgggccttggtctcatgggaagtggaatcgtctccaacttgctaaaaatgggtcacacagtgactgtctggaaccgcactgcagagaaagagggggcccgtctgggaagaacccccgctgaagtcgtctcaacctgcgacatcactttcgcctgcgtgtcggatcccaaggcggccaaggacctggtgctgggccccagtggtgtgctgcaagggatccgccctgggaagtgctacgtggacatgtcaacagtggacgctgacaccgtcactgagctggcccaggtgattgtgtccaggggggggcgctttctggaagcccccgtctcagggaatcagcagctgtctaatgacgggatgttggtgatcttagcggctggagacaggggcttatatgaggactgcagcagctgcttccaggcgatggggaagacctccttcttcctaggtgaagtgggcaatgcagccaagatgatgctgatcgtgaacatggtccaagggagcttcatggccactattgccgaggggctgaccctggcccaggtgacaggccagtcccagcagacactcttggacatcctcaatcagggacagttggccagcatcttcctggaccagaagtgccaaaatatcctgcaaggaaactttaagcctgatttctacctgaaatacattcagaaggatctccgcttagccattgcgctgggtgatgcggtcaaccatccgactcccatggcagctgcagcaaatgaggtgtacaaaagagccaaggcgctggaccagtctgacaacgatatgtccgccgtgtaccgagcctacatacactaagctgtcgacaccccgccctcacccctccaatcccccctctgaccccctcttcctcacatggggtcgggggcctgggagttcattctggaccagcccacctatctccatttccttttatacagactttgagacttgccatcagcacagcacacagcagcacccttcccctgaggccggtggggaggggacaagtgtcagcaggattggcgtgtgggaaagctcttgagctgggcactggccccccggacgaggtggctgtgtgttcacacacacacacacacacacacacacacaggctctcgccccaggatagaagctgcccagaaactgctgcctggctttttttcttccgagcttgtcttatctcaaaccccttccagtcaaggaactagaatcagcaacgagagttggaagccttcccacagcttcccccagagcgaagaggctgtagtcatgtccccatcccccactggattccctacaaggagaggccttgggcccagatgagccagtacagactccagacagaggggcccttggggccctccaacctcaggtgatgagctgagaaagatgttcacgtctaagcgtccagtgtgcacccagcgctccatagacgcctttgtgaactgaaaagagactggcagagtcccgagaagatggggccctggctttccagggagtgcagcaagcagccggcctgcagacccagcctgaccaacgatgagcatttcttaggctcagctcttgatacggaaacgagtgtcttcactccagccagcatcatggtcttcggtgcttcccgggcccggggtctgtcgggagggaagagaactgggcctgacctacctgaactgactggccctccgaggtgggtctgggacatcctagaggccctacatttgtccttggataggggaccggggggggcttggaatgttgcaaaaaaaaaagttacccaagggatgtcagttttttatccctctgcatgggttggattttccaaaatcataatttgcagaaggaaggccagcatttacgatgcaatatgtaattatatatagggtggccacactagggcggggtccttcccccctcacagctttggcccctttcagagattagaaactgggttagaggattgcagaagacgagtggggggagggcagggaagatgcctgtcgggtttttagcacagttcatttcactgggattttgaagcatttctgtctggacacaaagcctgttctagtcctggcggaacacactgggggtgggggcgggggaagatgcggtaatgaaaccggttagtcaattttgtcttaatattgttgacaattctgtaaagttcctttttatgaatatttctgtttaagctatttcacctttcttttgaaatccttcccttttaaggagaaaatgtgacacttgtgaaaaagcttgtaagaaagcccctcccttttttctttaaacctttaaatgacaaatctaggtaattaaggttgtgaatttttatttttgctttgtttttaatgaacatttgtctttcagaataggattgtgtgataatgtttaaatggcaaaaacaaaacatgattttgtgcaattaacaaagctactgcaagaaaaataaaacacttcttggtaacacaaaaaaaaaaaaaaa

SEQ ID NO: 137 - protein, 547 aa, Homo sapiens
MAAVSLRLGDLVWGKLGRYPPWPGKIVNPPKDLKKPRGKKCFFVKFFGTEDHAWIKVEQLKPYHAHKEEMIKINKGKRFQQAVDAVEEFLRRAKGKDQTSSHNSSDDKNRRNSSEERSRPNSGDEKRKLSLSEGKVKKNMGEGKKRVSSGSSERGSKSPLKRAQEQSPRKRGRPPKDEKDLTIPESSTVKGMMAGPMAAFKWQPTASEPVKDADPHFHHFLLSQTEKPAVCYQAITKKLKICEEETGSTSIQAADSTAVNGSITPTDKKIGFLGLGLMGSGIVSNLLKMGHTVTVWNRTAEKEGARLGRTPAEVVSTCDITFACVSDPKAAKDLVLGPSGVLQGIRPGKCYVDMSTVDADTVTELAQVIVSRGGRFLEAPVSGNQQLSNDGMLVILAAGDRGLYEDCSSCFQAMGKTSFFLGEVGNAAKMMLIVNMVQGSFMATIAEGLTLAQVTGQSQQTLLDILNQGQLASIFLDQKCQNILQGNFKPDFYLKYIQKDLRLAIALGDAVNHPTPMAAAANEVYKRAKALDQSDNDMSAVYRAYIH

SEQ ID NO: 138 - DNA, 1097 bp, Homo sapiens
attggttcaagaagaaaatttgagcccaggcacccaaacaccttcaaatgataaagcaagtatgttgcaagaatactccaaatacctccaacaggcttttgaaaaatccactaatgcaagttttactcttggacacggtttccaatttgtcagtttgtcttcacctctccacaaccacactttgtttccagaaaaacaaatatacactacgtctcctttggagtgtggtttcggccaatctgttacctcagtgttgccatcttcattgccaaagcctccttttgggatgttgtttggatctcagccaggtctttatttgtctgctttggatgctacacatcagcagttgacaccttcccaggagctggatgatctgatagattctcagaagaacttagagacttcatcagccttccagtcctcatctcagaaattgactagccagaaggaacagaaaaacttagagtcttcaacaggctttcagattccatctcaggagttagctagccagatagatcctcagaaagacatagagcctagaacaacgtatcagattgagaactttgcacaagcgtttggttctcagtttaagtcgggcagcagggtgccaatgacctttatcactaactctaatggagaagtggaccatagagtaaggacttcagtgtcagatttctcagggtatacaaatatgatgtctgatgtaagtgagccatgtagtacaagagtaaagacacccaccagccagagttacaggtaaggtcccaaaagtggccaggctggaggttttttaatgtaattttgttttattttgagaacactgccattggaatgtttttacacgatcctattaagaataatgtgatgccctttcaatgcaacttttcatatttagtttattttgttagcgtgattttagctctgtttgtattatgatttttaatcaaaatcaatagattaaaaatagtttgacattcaaagtgacaatgtttagcaatcaaatttacatgtatagattgtcagggaatagcccaaatgttttaaacgcaaaaaaaaaaaaaaaaaaaaaaaaaaaaaaaaaaaaaaaaaaaaaaaaa

SEQ ID NO: 139 - protein, 232 aa, Homo sapiens
MLQEYSKYLQQAFEKSTNASFTLGHGFQFVSLSSPLHNHTLFPEKQIYTTSPLECGFGQSVTSVLPSSLPKPPFGMLFGSQPGLYLSALDATHQQLTPSQELDDLIDSQKNLETSSAFQSSSQKLTSQKEQKNLESSTGFQIPSQELASQIDPQKDIEPRTTYQIENFAQAFGSQFKSGSRVPMTFITNSNGEVDHRVRTSVSDFSGYTNMMSDVSEPCSTRVKTPTSQSYR

SEQ ID NO: 140 - DNA, 775 bp, Homo sapiens
gtgtcacataccactcttgtaggtgtcctcaataatccccttttcccacaaaatacacagggtgtattatctttctctttattcacccccactttgctgaactgaagttaattacatagcctttcttctaacctccttagtaatgaaccttcacataaagtgtatttacagcgtctgtggtagccagcccttcctcctctactttctaggaggggatagccaataactaggaatttaatgacagattttttttttctttgaaataaatggccagagtttctccattttagaattttgttgtcctccttaatcatctgcttacctagtcattactcaatctgcagaaacttcataaaggaaaagtgctgcattgtttttacaaataacagtttgtagggaaaatatgacaaacctcaactatgggagttgtccacaatacaaaattttgaaaaaacattacatagtgataatatcatacttggttgttaggcttgttgcttccccacatcagaggcatctaatgatttatcttttgtaattgctgtgaacttttttaaataagccatttagtgtgaaattgtcatgtatcaaatggctattggaaatggactttactcaattttaattccactgcactctagccggagtgacagagtaagactctgtctcaaaaataaataaataaataaataaataaataaataaataaataaataaaaaataataatacaagttttcataaggaaaaaaaaaaaaaaaaaaaaaaaaaaaaaaa

SEQ ID NO: 141 - protein, 44 aa, Homo sapiens
MTNLNYGSCPQYKILKKHYIVIISYLVVRLVASPHQRHLMIYLL

SEQ ID NO: 142 - DNA, 2060 bp, Homo sapiens
gaaaaagaagacaaagctcaccttcaggcggaggtgcagcacctgcgagaggacaacctgaggctacaggaggagtcccagaacgcctcggacaagctgaagaagttcacagaatgggtcttcaacaccatagacatgagctagggaaggctgaggaggacaggagaagggcccagacactccctccagtgagtgtcctgcagcccttattccctccatagaaagcatcctcagagcaccttccctggcttcctactctgccccctttcggggagtgcacaacacaatagttgcagatcaacaatcatcacctgccttttgtagaaaagaaaaacaaaaaaagtaaataaaaattttaaacagtaaaataaaagtttaactgctaaaatgtgaatgtctttatttttttgcacaatatctttatctgttatgtatttaagaagaaactgggccttggaccagggcgccccctggcccatccgcctctattcccatcagctttcttatcaacttcaggtaacccaagctttcccttgttattctaacaaatatcattattcctagaaaaagaatgtttttataacttgtttggggagtagagagggatatttccttaccttcttccctaaaatgcctggagagggagttgctttgagaaaatgcctacctcccttgaatgactcgtgcatgagctagtgctgtctgtacctgtcctccagagatcagcaggaccggagttaaatatttaacagcaagtctgtaaaccagagcagctctgacagtgcctgcaggccacaccccttctcagtcctgcattgtgaggtcatttcctgcttctccctttccccaggaagatggtcccacttgtgctgcaagactcttttttgtttggcttaattgagccccactaaattggaatcaatctctcttacagcttcctggctccaaacattaattgatttcagaattcccccaaactaaaaccttatctgtctgcattttgaatgcattttggtcaaaagtatacgttttaaagatttttaaagataaaaatgtggcacaactggtttttttagcttgctgaaaatgaccatatctctaaattaatctttctctccagagcaagacttcaccagtatttgtaactaggagaagctaagtgaatgtttaattgtgaattttaatcattgcttgttaggaataatgactgtgatactagaatgggcttttgaaacctgcatgtcccagtgtgaaatttcagcacggcattttctgcatcctttcatggccatccaaaggattccgctgcagaaattattgatgtgctatttttgctgtcttgtgatgcaggctgctttgggcccctgggtcactcttccaaggctgctgtagagcacagagacatggggctggccagtgttgactgacctgaggagacccctttgtttgttgccttcataactgtcactaaaccgacccctctgccctttcagtggcaactctggtctaagggaacattcagcactctagcggcatctgattggaagttccctcacccaagtaatctcaattccttcctctctccatccctgaaagaaacaggatggattttcctctcttctccctgctacattcactaccagatttttatgctacagtttcattcttgattgtgatttctccatggaattttttttttctggtgacatttctatcatggaaataggaagatttcggagtgctttgtgaagatttcaattgtctgtctctttctctctttgacttgtatgaaggagattgtacattgcctgatatctctttgtaaatgagaaatattgctaacatccaagcattctgaagtcttgcttatccttctgagtttagttctcattttgttttacattttgtttggggacttggggcaagctatttattagagttttgcaacagagttcttgtttgaagcctctaaagactacctgtaaaattcaaagaataaaattcattttaaacgctcttttaaaaaaaaaaaaaaaaaaaaaaaaaaaaaaaaaaaaaaa

SEQ ID NO: 143 - protein, 62 aa, Homo sapiens [sequence omitted]

SEQ ID NO: 144 - DNA, 1160 bp, Homo sapiens
gattattttcagtaggcagacatctaatcggaatcttgctcttgttgcccaggctggagtgtaatggcacaatctcggcttactgcaacctctgcctcctggattcaagtgattctcctgcctcagcctcccaagtagctgggattacagccctgaaaaccactcgcttgcagagcgctggatcagcaatgcctactagttcttcattcaaacaccggattaaagagcaggaagactacatccgagattggactgctcatcgagaagagatagccaggatcagccaagatcttgctctcattgctcgggagatcaacgatgtagcaggagagatagattcagtgacttcatcaggcactgcccctagtaccacagtaagcactgctgccaccacccctggctctgccatagacactagagaagagttggttgatcgtgtttttgatgaaagcctcaacttccaaaagattcctccattagttcattccaaaacaccagaaggaaacaacggtcgatctggtgatccaagacctcaagcagcagagcctcccgatcacttaacaattacaaggcggagaacctggagcagggatgaagtcatgggagataatctgctgctgtcatccgtctttcagttctctargaagataagacaatctatagataagacagctggaaagatcagaatattatttaaagacaaagatcggaattgggatgacatagaaagcaaattaagagccgaaagtgaagtccctattgtgaaaacctcgagcatggagatttcttctatcttacaggaactgaaaagagtagaaaagcagctacaagcaatcaatgctatgattgatcctgatggaactttggaggctctgaacaacatgggatttcccagtgctatgttgccatctccaccgaaacagaagtccagccctgtgaataaccaccacagcccgggtcagacaccaacacttggccaaccagaagctagggctcttcatcctgctgctgtttcagccgcagctgaatttgagaatgctgaatctgaggctgatttcagtatacatttcaatagagtcaaccctgatggggaagaggaagatgttacagtaacataaatgactttctcttgattgttgaaaaaaaaaaaaaaaaaaaaa

SEQ ID NO: 145 - protein, 309 aa, Homo sapiens
  UNSURE (152)
MPTSSSFKHRIKEQEDYIRDWTAHREEIARISQDLALIAREINDVAGEIDSVTSSGTAPSTTVSTAATTPGSAIDTREELVDRVFDESLNFQKIPPLVHSKTPEGNNGRSGDPRPQAAEPPDHLTITRRRTWSRDEVMGDNLLLSSVFQFSXKIRQSIDKTAGKIRILFKDKDRNWDDIESKLRAESEVPIVKTSSMEISSILQELKRVEKQLQAINAMIDPDGTLEALNNMGFPSAMLPSPPKQKSSPVNNHHSPGQTPTLGQPEARALHPAAVSAAAEFENAESEADFSIHFNRVNPDGEEEDVTVT

SEQ ID NO: 146 - DNA, 1536 bp, Homo sapiens
  unsure (317)
gaagagagaaaatcagcctgtctgctctctccttggctcaacaaggcctctaacagtcttctgtcctctattctgcacacggcatatttgggaacgagaaacaaaagttttcccaaatgaagagaactcacttgtttattgtggggatttattttctgtcctcttgcagggcagaagaggggcttaatttccccacatatgatgggaaggaccgagtggtaagtctttccgagaagaacttcaagcaggttttaaagaaatatgacttgctttgcctctactaccatgagccggtgtcttcagataaggtcacgcanaaacagttccaactgaaagaaatcgtgcttgagcttgtggcccacgtccttgaacataaagctataggctttgtgatggtggatgccaagaaagaagccaagcttgccaagaaactgggttttgatgaagaaggaagcctgtatattcttaagggtgatcgcacaatagagtttgatggcgagtttgcagctgatgtcttggtggagttcctcttggatctaattgaagacccagtggagatcatcagcagcaaactggaagtccaagccttcgaacgcattgaagactacatcaaactcattggctttttcaagagtgaggactcagaatactacaaggcttttgaagaagcagctgaacacttccagccttacatcaaattctttgccacctttgacaaaggggttgcaaagaaattatctttgaagatgaatgaggttgacttctatgagccatttatggatgagcccattgccatccccaacaaaccttacacagaagaggagctggtggagtttgtgaaggaacaccaaaggtgcctgagatggcatgtgggggctgggggcctggggtctggggaatggagaggagcctctctgtgctaacatttcagacctgccaagagcaacaacctagttagtaccccagcagtacagaactcagtagtatggctttgttgatcagtaatgactagcagggatgttattacttctgaatctaagtctgcacctgcaagcagagtttgataaatccctcagtcagcaaatcccctcaaagccagggcaagatataaataaaattctatactaggaatgagagcaatttagtgaaagttcccatataccaataaccatgcccagtgctttagggaaactattttatctaatctccaaccttagggagtaattattattatcccaattttacagatcaaggaattggactcaatagttaagtaacttagccaaggatgaacactctatgcatagaacttctgggagagaaatgcttgataccacttagtgtagctccagcatggatcagcaaactttttctgtaaagaacaaaatggtaaatatttcaggttctgtgggccagatggcgtctgtagcaactacttaactgcggctgtggcatgaaagcagccatggatcatgtataaacaaatgggtgtggctgtgtaccagtaaaagtttatttaggaaaaaaaaaaaaaaa

SEQ ID NO: 147 - protein, 268 aa, Homo sapiens
  UNSURE (67)
MKRTHLFIVGIYFLSSCRAEEGLNFPTYDGKDRVVSLSEKNFKQVLKKYDLLCLYYHEPVSSDKVTXKQFQLKEIVLELVAHVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRCLRWHVGAGGLGSGEWRGASLC

SEQ ID NO: 148 - DNA, 1009 bp, Homo sapiens
gaattcggccttcatgcgcctgcaggaaagaatctgacatcatcacactgtgttttccttaacttgacaggaagtcaacttcaagcagattgacttgaaacgggatctcatttaggaagcataagtgtccaatcaaaaactgtgtatttttttaaatttggaaaatactcaagttccagttgcttatcattctccttcactttctgaaaacctggcaatcccatgtggacttctggtagaatgagcaatgcaaagaactggcttggacttggcatgtccttgtacttctgggggctgatggaccttacgaccaccgttctctcggacaccccaacaccacaaggtgaattagaagcactcctgtcagacaagccacagtcacatcagcggaccaagargagctgggtttggaaccagtttttcgttctggaagagtacactgggaccgaccctttgtatgtcggcaaggtaagaaatgccaagtagaaatgacccgggtagtggatattgaaattgaatatgaattgagtatcaaagttgacctagcctttatytgagacctgagaaaaactagaacaagtggtacgttacttgacacctagctaaaatgtaacttctgctttgtcagagaccagtctgaaaggaaagatttatttccttgtccatgtctctggtatgaatgggaaaaagtgggaattgggatttggaggaaaaggctcagaccctgcaagagctattcaagtcctaaaagaggcagcagcagctgtctgggaatgacagaatgggggagagggaaacttggaaatacaagaagagtacagagttttttgctttgtgtttttatggggtttttttcagagcatttccagaggtattgcctgaggtgcaaatttaatgaaaaaaataaaaataaaacatttttcatttcagaagatcttagcatgtgctttaggatagttggagacaataaatatatttataaatgttaaaaaaaaaaaaaaaaaaaaaa

SEQ ID NO: 149 - protein, 87 aa, Homo sapiens
  UNSURE (59)
MWTSGRMSNAKNWLGLGMSLYFWGLMDLTTTVLSDTPTPQGELEALLSDKPQSHQRTKXSWVWNQFFVLEEYTGTDPLYVGKVRNAK

SEQ ID NO: 150 - DNA, 2546 bp, Homo sapiens
aaaagaaaccaaggaaatttgtatgataaggcaggtaaagtgaggaaacatgcaactgaacaggaaaaaactgaagagggattaggccctaatataaaaagcattgtcaccatgttgatgctgatgctattgatgatgtttgctgtccactgtacctgggtcacaagcaatgcctactctagtccaagtgtagtcctggcctcatacaatcatgatggcaccaggaatatcttagatgattttagagaagcttacttttggctaaggcaaaatacagatgaacatgcacgagtaatgtcttggtgggattatggctatcagatagctggaatggctaatagaactacgttggtggataataacacctggaataacagccacatagcactggtgggaaaagctatgtcttctaatgaaacagcagcctataaaatcatgaggactctagatgtagattatgttttggttatttttggaggggttattggctattctggtgatgatatcaacaaatttctctggatggttaggatagctgaaggagaacatcccaaagacattcgggaaagtgactattttaccccacagggagaattccgtgtagacaaagcaggatcccctactttgttgaattgccttatgtataaaatgtcatactacagatttggagaaatgcagctggattttcgtacacccccaggttttgaccgaacacgtaatgctgagattggaaataaggacattaaattcaaacatttggaagaagcctttacatcagaacactggcttgttaggatatataaagtaaaagcacctgataacagggagacattagatcacaaacctcgagtcaccaacattttcccaaaacagaagtatttgtcaaagaagactaccaaaaggaagcgtggctacattaaaaataagctggtttttaagaaaggcaagaaaatatctaagaagactgtttaaatgcactgttctggttcctaacttgaagcagttgtccttgtgagaaccggtctttgcctttagctcatgtcgtgtttcacagcaaagagggtacagaaccatcactggtccaggttaatgtacaaaattttctggcaatgcctgattaaaaaaataaaattggcttgttgagaacagctgttttcgatttctaatgtgaagcaagacagagcactgctgtaaatgtctagcagcagattttttttttattggtacatattatccttcaaatctgagaatttggactaactgcaccaaagaaccctctaatttggtccctggcacatgcatacttgtcaatgtttttattctcttacaagacctgcattttatttgaattacccgaatagcaatatgtaaaatacaagtgacaaaatgtgatgagagcttcttgaaccggtaaactagtacaggtctgagaaagacatattagaagaatcattatacttccctgaattatatttattttcatgtttctctaatgcaaagaatgtttcatcaaatgtatattttctgttgcttactgtttgctctgagaagaagctgctgtttcaaagatggacctctgagtagctaattgattcaagtagtttttttatgttgacacattattactgctgttagcagtcgttttcaccaggtacttacagagcagatttcatacatcattcattcaagggctaaatttatattttttggaaatcatggcaactacacaggatgttgcttaccaggacggagttttggtatcttagtactgaagttagcactatgtttacatgcaaaagattaaggaaaaaacccttaaagtggacaggtatccaaagttcattttctgtgactcatcaaagtgacaaaagacttgtaacaactttgcctggacttttttcattttacaacagttcatccattcacaatgattttgttctctgctccatattttttaatcccttaagcatttgatgaaacactctttagtgctatatgcattttcttacttttgttaaaaatgtgacaattgtcaaaaaatgcactaaaatgtaaatggagattgaacaagttcactttccagcttataggcaactttatacagacttgaacattttctccagttgtttagtaaaagtgaaagagaaagggtttttcctgccacaggatataacttttttttatataacaagcataacacaccactgcttttggtggaaaagtgcagaatagtatgtaccttttatgaagaaaaatgtaatttacaatattcagtgagaatgttactgctgattttcttttccaaggtgtagaatattctttgatttatagaattcatttttgacccagatgatggttcctttacagaacaataaaatggctgaacattttcacaaatagagtgtaacgaagtctggatttctgataccttgtcatttgggggattttattttactttgttgctttaaaattcaatgcagagaagttgttgactgtaggggaaataaagttaattcaaattttgaaaaaaaaaaaaaaaaaaaaaa

SEQ ID NO: 151 - protein, 286 aa, Homo sapiens
MLMLMLLMMFAVHCTWVTSNAYSSPSVVLASYNHDGTRNILDDFREAYFWLRQNTDEHARVMSWWDYGYQIAGMANRTTLVDNNTWNNSHIALVGKAMSSNETAAYKIMRTLDVDYVLVIFGGVIGYSGDDINKFLWMVRIAEGEHPKDIRESDYFTPQGEFRVDKAGSPTLLNCLMYKMSYYRFGEMQLDFRTPPGFDRTRNAEIGNKDIKFKHLEEAFTSEHWLVRIYKVKAPDNRETLDHKPRVTNIFPKQKYLSKKTTKRKRGYIKNKLVFKKGKKISKKTV

SEQ ID NO: 152 - DNA, 4061 bp, Homo sapiens
aagcaattgaagaaattgcagcaggatgtgatggaaatgaaaaaaacaaaggttcgcctaatgaaacaaatgaaagaagaacaagagaaagccagactgactgagtctagaagaaacagagagattgctcagttgaaaaaggatcaacgtaaaagagatcatcmacttagacttctggaagcccaaaaaagaaaccaagaagtggttctacgtcgcaaaactgaagaggttacggctcttcgtcggcaagtaagacccatgtcagataaagtggctgggaaagttactcggaagctgagttcatctgatgcacctgctcaggacacaggttccagtgcagctgctgtcgaaacagatgcatcaaggacaggagcccagcagaaaatgagaattcctgtggcgagagtccaggccttaccaacgccggcaacaaatggaaacaggaaaaaatatcagaggaaaggattgactggccgagtgtttatttccaagacagctcgcatgaagtggcagctccttgagcgcagggtcacagacatcatcatgcagaagatgaccatttccaacatggaggcagatatgaatagactcctcaagcaacgggaggaactcacaaaaagacgagagaaactttcaaaaagaagggagaagatagtcaaggagaatggagagggagataaaaatgtggctaatatcaatgaagagatggagtcactgactgctaatatcgattacatcaatgacagtatttctgattgtcaggccaacataatgcagatggaagaagcaaaggaagaaggtgagacattggatgttactgcagtcattaatgcctgcacccttacagaagcccgatacctgctagatcacttcctgtcaatgggcatcaataagggtcttcaggctgcccagaaagaggctcaaattaaagtactggaaggtcgactcaaacaaacagaaataaccagtgctacccaaaaccagctcttattccatatgttgaaagagaaggcagaattaaatcctgagctagatgctttactaggccatgctttacaagatctagatagcgtaccattagaaaatgtagaggatagtactgatgaggatgctcctttaaacagcccaggatcagaaggaagcacgctgtcttcagatctcatgaagctttgtggtgaagtgaaacctaagaacaaggcccgaaggagaaccaccactcagatggaattgctgtatgcagatagcagtgaactagcttcagacactagtacaggagatgcctccttgcctggccctctcacacctgttgcagaagggcaagagattggaatgaatacagagacaagtggtacttctgctagggaaaaagagctctctcccccacctggcttaccttctaagataggcagcatttccaggcagtcatctctatcagaaaaaaaaattccagagccttctcctgtaacaaggagaaaggcatatgagaaagcagaaaaatcaaaggccaaggaacaaaagcactcagattctggaacttcagaggctagtctttcacctccttcttccccaccaagccggccccgtaatgaactgaatgtttttaatcgtcttactgtttctcagggaaacacatcagttcagcaggataagtctgatgaaagtgactcctctctctcggaggtacacagcagatcctccagaaggggcataatcaacccatttcctgcttcaaaaggaatcagagcttttccacttcagtgtattcacatagctgaagggcatacaaaagctgtgctctgtgtggattctactgatgatctcctcttcactggatcaaaagatcgtacttgtaaagtatggaatctggtgactgggcaggaaataatgtcactggggggtcatcccaacaatgtcgtgtctgtaaaatactgtaattataccagtttggtcttcactgtatcaacatcttatattaaggtgtgggatatcagagattcagcaaagtgcattcgaacactaacgtcttcaggtcaagttactcttggagatgcttgttctgcaagtaccagtcgaacagtagctattccttctggagagaaccagatcaatcaaattgccctaaacccaactggcaccttcctctatgctgcttctggaaatgctgtcaggatgtgggatcttaaaaggtttcagtctacaggaaagttaacaggacacctaggccctgttatgtgccttactgtggatcagatttccagtggacaagatctaatcatcactggctccaaggatcattacatcaaaatgtttgatgttacagaaggagctcttgggactgtgagtcccacccacaattttgaaccccctcattatgatggcatagaagcactaaccattcaaggggataacctatttagtgggtctagagataatggaatcaagaaatgggacttaactcaaaaagaccttcttcagcaagttccaaatgcacataaggattgggtctgtgccctgggagtggtgccagaccacccagttttgctcagtggctgcagagggggcattttgaaagtctggaacatggatacttttatgccagtgggagagatgaagggtcatgatagtcctatcaatgccatatgtgttaattccacccacatttttactgcagctgatgatcgaactgtgagaatttggaaggctcgcaatttgcaagatggtcagatctctgacacaggagatctgggggaagatattgccagtaattaaacatgaatgaagataggttgtaaactgaatgctgtgataatactctgtattctttatggaaaatgttgtcctgtacttactaggcaaaacgtatgaatcggattaactggaaaatatatctgaattcaactgctgactataaatggtattctaataaaattgtgtactatcctgtgtgcttagttttaagatcaaccaatagatatatatcctacaattgatatattgctttattcacacttttattgtggctgaatttttgtgcctatctataaaacacactttcaaattatttgaattaccaagacgtctgcttttgtgacagtcagaaaacacacctggaatacgatgcagcccaccattaactcattcatgtagtttattcaagtgatttatgtatttaaactaaatattgaaaatgttagtcaaattgtggtttgcttgtcaggtatttatatcagtctgtagtggattcccaaatttcaaagctcttttaatgtaatggacaaaaataagatatgagaatattattgatgaattttcataaggtggaattgatcttaatctactaacagagaagggtagacagtttgtgttaaatgttggcatttacttgtattgaccaaagttttgcagctctactatattctgtgctcaggactaaaatgctgttaatttttttttttttttttccagtgctgtgcatatattctgtgatgggaaacattgttgatgtcctaacagaaatatattttgatctattttcctatggagttgtttctattatgaccatttaattttgtttttatttaatagtagtatttccttcccttttatctaattttttatatgctgctaaatatattttaaatatactatgtttgcgaaccttggtagctatgatgagagctattatcatctgtggtgggaaaagctatgtaaataggtagattgtatagagagactatcttgtgttgtgcctgtatgaatttttaaaagttgttgactggattttgcaaaaggatgtataatatttctgtctgctcagaatattaatttgtaaattctgcaagtttaatttttatgtagatggtataacatttgaaaatattgtcttatgtgattttttcccctgaaaatatttgcttgtaaatgaaaacttagctagggcttaaataaacatgttgctatgaaatkraaaaaaaaaaaaaaaaaraaaaaaaaaaaaaaaaaaaaaaaaaaaaaaaaaaaaaraaaaaaaaaaaaaaaaaaaaaaaaaaaaaaaaaaaaaa

SEQ ID NO: 153 - protein, 910 aa, Homo sapiens
  UNSURE (43)
MKKTKVRLMKQMKEEQEKARLTESRRNREIAQLKKDQRKRDHXLRLLEAQKRNQEVVLRRKTEEVTALRRQVRPMSDKVAGKVTRKLSSSDAPAQDTGSSAAAVETDASRTGAQQKMRIPVARVQALPTPATNGNRKKYQRKGLTGRVFISKTARMKWQLLERRVTDIIMQKMTISNMEADMNRLLKQREELTKRREKLSKRREKIVKENGEGDKNVANINEEMESLTANIDYINDSISDCQANIMQMEEAKEEGETLDVTAVINACTLTEARYLLDHFLSMGINKGLQAAQKEAQIKVLEGRLKQTEITSATQNQLLFHMLKEKAELNPELDALLGHALQDLDSVPLENVEDSTDEDAPLNSPGSEGSTLSSDLMKLCGEVKPKNKARRRTTTQMELLYADSSELASDTSTGDASLPGPLTPVAEGQEIGMNTETSGTSAREKELSPPPGLPSKIGSISRQSSLSEKKIPEPSPVTRRKAYEKAEKSKAKEQKHSDSGTSEASLSPPSSPPSRPRNELNVFNRLTVSQGNTSVQQDKSDESDSSLSEVHSRSSRRGIINPFPASKGIRAFPLQCIHIAEGHTKAVLCVDSTDDLLFTGSKDRTCKVWNLVTGQEIMSLGGHPNNVVSVKYCNYTSLVFTVSTSYIKVWDIRDSAKCIRTLTSSGQVTLGDACSASTSRTVAIPSGENQINQIALNPTGTFLYAASGNAVRMWDLKRFQSTGKLTGHLGPVMCLTVDQISSGQDLIITGSKDHYIKMFDVTEGALGTVSPTHNFEPPHYDGIEALTIQGDNLFSGSRDNGIKKWDLTQKDLLQQVPNAHKDWVCALGVVPDHPVLLSGCRGGILKVWNMDTFMPVGEMKGHDSPINAICVNSTHIFTAADDRTVRIWKARNLQDGQISDTGDLGEDIASN

SEQ ID NO: 154 - DNA, 372 bp, Homo sapiens
aatcgcacctgtccaacttgcaatgacttccatggacttgtgcagaaaatcatggagctacaggatattttagccaaaacatcagccaagctgtctcgagctgaacagcgaatgaatagattggatcagtgctattgtgaaaggacttgcaccatgaagggaaccacctaccgagaatttgagtcctggatagacggctgtaagaactgcacatgcctgaatggaaccatccagtgtgaaactctaatctgcccaaatcctgactgcccacttaagtccgctcttgcgtatgtggatggcaaatgctgtaaggaatgcaaatcgatattccaatttcaaggacgaacctactttgaaggagaaagaaataca

SEQ ID NO: 155 - DNA, 761 bp, Homo sapiens
  unsure (108)
  unsure (191)
  unsure (268)
  unsure (299)
ttctggagtatgtgttctctatgagtgcaaggaccagaccatgaaatttgttgagagttcaggctgtccagctttggattgtccagagtctcatcagataaccttgtntcacagctgttgcaaagtttgtaaaggttatgatttttgttttgaaaggcataactgcatggagaattccatctgcagaaatntgaatgacagggctgtttgtagctgtcgagatggttttagggtttttcgagaggataatgcctactgtgaagacatngatgagtgtgctgaagggcgccattactgtngtgaaaatacaatgtgtgtcaacaccccgggttcttttatgtgcatctgcaaaactggatacatcagaattgatgattattcatgtacagaacatgatgagtgtatcacaaatcagcacagctgtgatgaaaatgctttatgcttcaacactgttggaggacacaactgtgtttgcaagccgggctatacagggaatggaacgacatgcaaagcattttgcaaagatggctgtaggaatggaggagcctgtattgccgctaatgtgtgtgcctgcccacaaggcttcactggacccagctgtgaaacggacattgatgaatgctctgatggttttgttcaatgtgacagtcgtgctaattgcattaacctgcctggatggtaccactgtgagtgcagagatggctaccatgacaatgggatgttttcaccaagtggagaatcgtgtgaagatattgatgagt

SEQ ID NO: 156 - protein, 240 aa, Homo sapiens
  UNSURE (23)
  UNSURE (51)
  UNSURE (76)
  UNSURE (87)
MKFVESSGCPALDCPESHQITLXHSCCKVCKGYDFCFERHNCMENSICRNXNDRAVCSCRDGFRVFREDNAYCEDXDECAEGRHYCXENTMCVNTPGSFMCICKTGYIRIDDYSCTEHDECITNQHSCDENALCFNTVGGHNCVCKPGYTGNGTTCKAFCKDGCRNGGACIAANVCACPQGFTGPSCETDIDECSDGFVQCDSRANCINLPGWYHCECRDGYHDNGMFSPSGESCEDIDE

SEQ ID NO: 157 - DNA, 342 bp, Homo sapiens
gcagaaaattttcctctagatcagaatcttcaagaatcagttaggttcctcactgcaagaaataaaatgtcaggcagtgaatgaattatattttaagaagtaaagcaaagaagctataacatgttatgtacagtacactctgaaaagaaatctgaaacaagttattgtaatgataaaaataatgcacaggcatggttacttaatattttctaacaggaaaagtcatccctatttccttgttttactgcacttaatattatttggttgaatttgttcagtataagttcgttccttgtgcaaaattaaataaatatttttcttaccttaaaaaaaaaaaaaaaa

SEQ ID NO: 158 - DNA, 1445 bp, Homo sapiens
gtgcgcatggggacgctatagcaattcgtttgctgtccttcctctccttcgaagatgacaaggcctaccatcgtttcttcctgcctttgggccgtcaggcagttggttgggacccgctccaaccctcggttcttcctgcaatacagtggatacaatttgtcatggctactctgagataagaccacttttttatctgagcttctgtgacctgctcctgggactttgctggctcacggagacacttctctatggagcttcagtagcaaataaggacatcatctgctataacctacaagcagttggacagatattctacatttcctcatttctctacaccgtcaattacatctggtatttgtacacagagctgaggatgaaacacacccaaagtggacagagcacatctccactggtgatagattatacttgtcgattttgtcaaatggcctttgttttctcaagcctgatacctctgctattgatgacacctgtattctgtctgggaaatactagtgaatgtttccaaaacttcagtcagagccacaattgtatcttgatgcactcaccaccatcagccatggctgaacttccaccttctgccaacacatctgtctgtagcacactttatttttatggtatcgccattttcctgggcagctttgtactcagcctccttaccattatggtcttacttatccgagcccagacattgtataagaagtttgtgaagtcaactggctttctggggagtgaacagtgggcagtgattcacattgtggaccaacgggtgcgcttctacccagtggccttcttttgctgctggggcccagctgtcattctaatgatcataaagctgactaagccacaggacaccaagcttcacatggccctttatgttctccaggctctaacggcaacatctcagggtctactcaactgtggagtatatggctggacgcagcacaaattccaccaactaaagcaggaggctcggcgtgatgcagatacccagacaccattattatgctcacagaagagattctatagcaggggcttaaattcactggaatccaccctgacttttcctgccagtacttctaccattttttgaaactacaatactggaacatccaggaactggagttattctacgctaatggattggaaagaatgttgggaaaggacatcttaaatcttttctaactatgccctaaactgcagaactcaaaggaaatatagtgccattgttagtagtcattctagatgaattgggagtatctctccagttattcccagattcactagtgatccttaaagtctctattcagggagaggaagacactttccatctcagagatagactcgtgttaccttgatggatattggatttgtctaagtctcttctagaaaaaataaattctagattattaaaaaaaaaaaaaaaa

SEQ ID NO: 159 - protein, 245 aa, Homo sapiens
MKHTQSGQSTSPLVIDYTCRFCQMAFVFSSLIPLLLMTPVFCLGNTSECFQNFSQSHNCILMHSPPSAMAELPPSANTSVCSTLYFYGIAIFLGSFVLSLLTIMVLLIRAQTLYKKFVKSTGFLGSEQWAVIHIVDQRVRFYPVAFFCCWGPAVILMIIKLTKPQDTKLHMALYVLQALTATSQGLLNCGVYGWTQHKFHQLKQEARRDADTQTPLLCSQKRFYSRGLNSLESTLTFPASTSTIF

SEQ ID NO: 160 - DNA, 3550 bp, Homo sapiens
cccctcgataatggattactaaatgggatacacgctgtaccagttcgctccgagccccggccgcctgtccgtcgatgcaccgaaaagggtgaagtagagaaataaagtctccccgctgaactactatgaggtcagaagccttgctgctatatttcacactgctacactttgctggggctggtttcccagaagattctgagccaatcagtatttcgcatggcaactatacaaaacagtatccggtgtttgtgggccacaagccaggacggaacaccacacagaggcacaggctggacatccagatgattatgatcatgaacggaaccctctacattgctgctagggaccatatttatactgttgatatagacacatcacacacsgaagaaatttattgtagcaaaaaactgacatggaaatctagacaggccgatgtagacacatgcagaatgaagggaaaacataaggatgagtgccacaactttattaaagttcttctaaagaaaaacgatgatgcattgtttgtctgtggaactaatgccttcaacccttcctgcagaaactataagatggatacattggaaccattcggggatgaattcagcggaatggccagatgcccatatgatgccaaacatgccaacgttgcactgtttgcagatggaaaactatactcagccacagtgactgacttccttgccattgacgcagtcatttaccggagtcttggagaaagccctaccctgcggaccgtcaagcacgattcaaaatggttgaaagaaccatactttgttcaagccgtggattacggagattatatctacttcttcttcagggaaatagcagtggagtataacaccatgggaaaggtagttttcccaagagtggctcaggtttgtaagaatgatatgggaggatctcaaagagtcctggagaaacagtggacgtcgttcctgaaggcgcgcttgaactgctcagttcctggagactctcatttttatttcaacattctccaggcagttacagatgtgattcgtatcaacgggcgtgatgttgtcctggcaacgttttctacaccttataacagcatccctgggtctgcagtctgtgcctatgacatgcttgacattgccagtgtttttactgggagattcaaggaacagaagtctcctgattccacctggacaccagttcctgatgaacgagttcctaagcccaggccaggttgctgtgctggctcatcctccttagaaagatatgcaacctccaatgagttccctgatgataccctgaacttcatcaagacgcacccgctcatggatgaggcagtgccctccatcttcaacaggccatggttcctgagaacaatggtcagataccgccttaccaaaattgcagtggacacagctgctgggccatatcagaatcacactgtggtttttctgggatcagagaagggaatcatcttgaagtttttggccagaataggaaatagtggttttctaaatgacagccttttcctggaggagatgagtgtttacaactctgaaaaatgcagctatgatggagtcgaagacaaaaggatcatgggcatgcagctggacagagcaagcagctctctgtatgttgcgttctctacctgtgtgataaaggttccccttggccggtgtgaacgacatgggaagtgtaaaaaaacctgtattgcctccagagacccatattgtggatggataaaggaaggtggtgcctgcagccatttatcacccaacagcagactgacttttgagcaggacatagagcgtggcaatacagatggtctgggggactgtcacaattcctttgtggcactgaatggagtgattcgggaaagttacctcaaaggccacgaccagctggttcccgtcaccctcttggccattgcagtcatcctggctttcgtcatgggggccgtcttctcgggcatcaccgtctactgcgtctgtgatcatcggcgcaaagacgtggctgtggtgcagcgcaaggagaaggagctcacccactcgcgccggggctccatgagcagcgtcaccaagctcagcggcctctttggggacactcaatccaaagacccaaagccggaggccatcctcacgccactcatgcacaacggcaagctcgccactcccggcaacacggccaagatgctcattaaagcagaccagcaccacctggacctgacggccctccccaccccagagtcaaccccaacgctgcagcagaagcggaagcccagccgcggcagccgcgagtgggagaggaaccagaacctcatcaatgcctgcacaaaggacatgccccccatgggctcccctgtgattcccacggacctgcccctgcgggcctcccccagccacatccccagcgtggtggtcctgcccatcacgcagcagggctaccagcatgagtacgtggaccagcccaaaatgagcgaggtggcccagatggcgctggaggaccaggccgccacactggagtataagaccatcaaggaacatttcagcagcaagagtcccaaccatggggtgaaccttgtggagaacctggacagcctgccccccaaagttccacagcgggaggcctccctgggtcccccgggagcctccctgtttcagaccggtttaagcaagcggctggaaatgcaccactccttttcctacggggttgactataagaggagctaccccacgaactcgctcacgagaagccaccaggccaccactctcaaaagaaacaacactaactcctccaattcctctcacctctccagaaaccagagctttggcaggggagacaacccgccgcccgccccgcagagggtggactccatccaggtgcacagctcccagccatctggccaggccgtgactgtctcgaggcagcccagcctcaacgcctacaactcactgacaaggtcggggctgaagcgtacgccctcgctaaagccggacgtaccccccaaaccatcctttgctcccctttccacatccatgaagcccaatgatgcgtgtacataatcccagggggagggggtcaggtgtcgaaccagcaggcaaggcgaggtgcccgctcagctcagcaaggttctcaactgcctcgagtacccaccagaccaagaaggcctgcggcagagccgaggacgctgggtcctcctctctgggacacaggggtactcacgaaaactgggccgcgtggtttggtgaaggtttgcaacggcggggactcaccttcattctcttccttcactttcccccacaccctacaacaggtcggacccacaaaagacttcagttatcatcacaaacatgagccaaaagcacatacataccccatcccccacccccacacacacacacacatgcacacaacacatacacacacacgcacagaggtgaacagaaactgaaacattttgtccacaacttcacgggacgtggccagactgggtttgcgttccaacctgcaaaacacaaatacattttttaaaatcaagaaaatttaaaaaaaaaaaaaa

SEQ ID NO: 161 - protein, 975 aa, Homo sapiens
MRSEALLLYFTLLHFAGAGFPEDSEPISISHGNYTKQYPVFVGHKPGRNTTQRHRLDIQMIMIMNGTLYIAARDHIYTVDIDTSHTEEIYCSKKLTWKSRQADVDTCRMKGKHKDECHNFIKVLLKKNDDALFVCGTNAFNPSCRNYKMDTLEPFGDEFSGMARCPYDAKHANVALFADGKLYSATVTDFLAIDAVIYRSLGESPTLRTVKHDSKWLKEPYFVQAVDYGDYIYFFFREIAVEYNTMGKVVFPRVAQVCKNDMGGSQRVLEKQWTSFLKARLNCSVPGDSHFYFNILQAVTDVIRINGRDVVLATFSTPYNSIPGSAVCAYDMLDIASVFTGRFKEQKSPDSTWTPVPDERVPKPRPGCCAGSSSLERYATSNEFPDDTLNFIKTHPLMDEAVPSIFNRPWFLRTMVRYRLTKIAVDTAAGPYQNHTVVFLGSEKGIILKFLARIGNSGFLNDSLFLEEMSVYNSEKCSYDGVEDKRIMGMQLDRASSSLYVAFSTCVIKVPLGRCERHGKCKKTCIASRDPYCGWIKEGGACSHLSPNSRLTFEQDIERGNTDGLGDCHNSFVALNGVIRESYLKGHDQLVPVTLLAIAVILAFVMGAVFSGITVYCVCDHRRKDVAVVQRKEKELTHSRRGSMSSVTKLSGLFGDTQSKDPKPEAILTPLMHNGKLATPGNTAKMLIKADQHHLDLTALPTPESTPTLQQKRKPSRGSREWERNQNLINACTKDMPPMGSPVIPTDLPLRASPSHIPSVVVLPITQQGYQHEYVDQPKMSEVAQMALEDQAATLEYKTIKEHFSSKSPNHGVNLVENLDSLPPKVPQREASLGPPGASLFQTGLSKRLEMHHSFSYGVDYKRSYPTNSLTRSHQATTLKRNNTNSSNSSHLSRNQSFGRGDNPPPAPQRVDSIQVHSSQPSGQAVTVSRQPSLNAYNSLTRSGLKRTPSLKPDVPPKPSFAPLSTSMKPNDACT

SEQ ID NO: 162 - DNA, 1723 bp, Homo sapiens
ctgcagactttggggtcaccggccagccacacaggcaccgttttcagatgtccacttctcattgggtacatcaatcttttaactttgggggtcacagttttagccacctttcggggggtgactggagcagtaggaggtgtggggtcattttatgaatataataaaatggagctgactatggacrrrgactwagtgtgggggagaggggacgatacagggtgtgtgtctgggagtgcctgggggacagggaccccccggtggtcctatggcaggatgagaarggagggacttggctcccccagagcccggtggaagctactgttctctccagtgtctcgagcgtagccaaaataaggttgggaggctcccggcctgtctgctgtggtctgagctggctgcaagcccaggtgggggagcgagtctgggaagattggctttgactctctgttgccagaggagatgccatcccagcacggcccccactgtagtccaggctcgtggtggcagcgggggcaaggggaggggcaaggctgcccccaccccacgcaccaagtcacgccaagtctcagcaggtaaaagcacgtgagcctagggcgagcggagggagtcctggtggccccgcaggtcaggagggaaagcagggctcagagggcatcgtggccccagggcagggtcctacctgggggtcaggagcaccttggtcttgatgattgattgattgatagaatggagctgggtctgagcctcccaggcttgagctcctgggagttcttgtgcggtgagctgggcagctcctgggtaggtccgggcaccaagcaggccctgatgtggacagagtcccatcagagggagctgatgaagaatggtccctgtaagtaagtcactaggttcaacaactgcctggccgagcactcagcccgtggagctcaggccaacaccagagccccggttttaggggccaggagagcaggtgaccaattatttggggagtcttgggtagaatttccgccacacattctccccagggctgcaggggtcttccgaggcagggcggtggagcaggattcaggatgtggtgggaatagagtgaggggcagtgggtgggcagacctgggcgtcagaggtcctgatgggaaaggaggcaggggctacccagagaggggggctcgtgtggcacagcccccaccgactccgccgtccccctcccctgtgagccccgggggctgtacatactctactccatccccttgtccatccctgagaccacccccgccgcccttgcgtcgacttagcaaccacctcataggcccacccacctcgggatccgagccaaccatcccacatcacaaactttggtttgggggactttacgttcgtttaatttctcattttgtacggagaaatattcttttcaaaagcgtcttttgactgaagtaactttcctggtgctgttgttaactcgttcctttttttaatttattcccccaccccaggcagccctcctggttcctactcaccctccccccctcccccaccctccgtcccatctgaaccatttgtttcttttctttccgtcagattttggaaaaattctcctctcctccccgccccctccacaccatcctcccsgatttaaatatagtcactgctacaagtaacagatgcactgtgaagattccagtattaataaaggtgtactgtaattaacaaaaaaaaaaaaaaa

SEQ ID NO: 163 - protein, 101 aa, Homo sapiens
  UNSURE (49)
  UNSURE (51)
  UNSURE (81)
VFRCPLLIGYINLLTLGVTVLATFRGVTGAVGGVGSFYEYNKMELTMDXDXVWGRGDDTGCVSGSAWGTGTPRWSYGRMRXEGLGSPRARWKLLFSPVSRA

SEQ ID NO: 164 - DNA, 469 bp, Homo sapiens
gcaacatacaagccggccatattagagagatggaaataaagcttccttaatgttgtatatgtctttgaagtacatccgtgcatttttttttagcatccaaccattcctcccttgtagttctcgccccctcaaatcaccctctcccgtagcccacccgactaacatctcagtctctgaaaatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaaga

SEQ ID NO: 165 - protein, 96 aa, Homo sapiens
MHRDAWLPRPAFSLTGLSLFFSLVPPGRSMEVTVPATLNVLNGSDARLPCTFNSCYTVNHKQFSLNWTYQECNNCSEEMFLQFRMKIINLKLERFQ

SEQ ID NO: 166 - DNA, 454 bp, Homo sapiens
tggcttttggctacagagagggaagggaaagcctgaggccggcataaggggaggccttggaacctgagctgccaatgccagccctgtcccatctgcggccacgatactcgctcctctcccaacaactcctttggtggggacaaaagtgacaattgtaggccaggcacagtggctcacgcctgtaatcccagcactttgggaggccaaggcgggtggattacctccatctgtttagtagaaatgggcaaaaccccatttttactaaaaatacaagaattagctgggcgtggtggcgtgtgcctgtaatcccagctatttgggaggctgaggcaggagaatcgcttgagcccgggaagcagaggttgcagtgaactgagatagtgatagtgccactgcaattcagcctgggtgacatagagagactccatctcaaaaaaaaaaaaaaaaaaaaaaa

SEQ ID NO: 167 - DNA, 736 bp, Homo sapiens
  unsure (680)
  unsure (704)
gtttttaaacattatgttctacatgataaatacatataatagtatgtctatttaaataattaaatttgaaaaaaactaatcaaatattatcataagtaatgataaaaaccacaatttcttttgcagcaaactaataacacctggatttctcaatttattaagttgtacttacctgatgctgatgatgattactgtatttacacattgtctcagagctcactcttgcggaggttgtggcctcgaaaatgccttgttgtccctctggaatctgtcttttcagcttcatctcctcctcctcacctcctgctgtggtgcacagatacctataggcaggctccatctcctcctccccagctcctcccctagtgcacagatacctataggcaggcttcatctcctcctccccagcttctcccctagtgcacagatacctataggcaggctccatctcctcctccccagctcctcccctartgcacagacacctataggcaagctccatctcctcctctttagctagcctccccatctcatcacaacgcatgtctgtgacctttggtaatcatttacagtgccacacggaaccctgtattttgcacacagcaaaacaaacaatgtttagctttatttatggtatttgatgactgtaaatggaaataaatattgttctttattttttnaaaaaaaaaaaaaaaaaaaaaaanaaaaaaaaaaaaaaaaaaaaaaaaaaaaaaaa

SEQ ID NO: 168 - protein, 114 aa, Homo sapiens
  UNSURE (100)
MLMMITVFTHCLRAHSCGGCGLENALLSLWNLSFQLHLLLLTSCCGAQIPIGRLHLLLPSSSPSAQIPIGRLHLLLPSFSPSAQIPIGRLHLLLPSSSPXAQTPIGKLHLLLFS

SEQ ID NO: 169 - DNA, 1427 bp, Homo sapiens
gtagttactaactccaacacctaatagcattggtagaaagcttataaatgcagttatttagcctcgactaagatttttctgatacctagtttcactttttaatgccctctgaaagttttttgatcagttgtttaatgggagatctgaaatgttaaactcagaccagaaagaagagaacctgttttctagaaattaggtttttaatccaagtaagatgcaagcttttgcttttttaataacttgtatagctaaaaacttgacggtgaaaagctctcagatcaaagctgatccttctgtcagtaatgattctaaaaataagcaagattttaatggggaatatattttatttcattcttatctcaaacctaggtactgtggtcgttttgagttcatttcgaggcattttcaatgtgcctcaggccacatccaacctctycccagggccagatttaatgttcagcctcataaaggttatcatagttttaacatttaagtactattttgcagtgggtatataccaaaatttgctaatagtaagataaccttagttatatatcattcacgttagttctatcttggaggcaataaacatttcttgttcaagaaattcatgttctatcttggaggcaataaacaaacattttttgttcaaaattagggctaccctattgtccttatgtcttttcctgatctgtggtcaaacatttttcttagtcatttagaaattttctatgttgttttaaattttctttaaatctagaatggagtatgtgaccaatactttcctttggaatggtatggacatttgaaatagagcccattctttataaagtataaaatatgtttaatgctagtatttttaactaaacttttgagaaactagattcacatgctgttgtaagaaataatacagagacctctttcgtgtacctttcactttgtttcccacacagtgaacatctttcaaaactgtcatacaatatcatacccaggatactgacactggtatagctaagatagagaacgtttccacacagaactttttctagcacagggatccctcatcttgcttttgatgaccatacccacttcactcccatccctactcccttcttaacccttggcaaccataatctgttctccatttttatagttttttttttttcatttcaataaagctgtataactggaatcataataatatgtaaccttttgggattggctttttttcatttagcatgattttctggaggttaatccagcttattatgtgtatcaagtctattgacaggtactttttagtgtgaatagaatcccatagtatagatgtaccacagtttgtttaactgttcacctgctgagagacattgggccagtttttggctactataaataaagttgctataaacaaaaaaaaaaaaaaaaaa

SEQ ID NO: 170 - protein, 79 aa, Homo sapiens
  UNSURE (45)
MILKISKILMGNIFYFILISNLGTVVVLSSFRGIFNVPQATSNLXPGPDLMFSLIKVIIVLTFKYYFAVGIYQNLLIVR

SEQ ID NO: 171 - DNA, 572 bp, Homo sapiens
tgcagattctgtggttatactcactcctcatcccaaagaatgaaatttaccactctcctcttcttggcagctgtagcaggggccctggtctatgctgaagatgcctcctctgactcgacgggtgctgatcctgcccaggaagctgggacctctaagcctaatgaagagatctcaggtccagcagaaccagcttcacccccagagacaaccacaacagcccaggagacttcggcggcagcagttcaggggacagccaaggtcacctcaagcaggcaggaactaaaccccctgaaatccatagtggagaaaagtatcttactaacagaacaagcccttgcaaaagcaggaaaaggaatgcacggaggcgtgccaggtggaaaacaattcatcgaaaatggaagtgaatttgcacaaaaattactgaagaaattcagtctattaaaaccatgggcatgagaagctgaaaagaatgggatcattggacttaaagccttaaatacccttgtagcccagagytattaaaacgaaagcatccaaaaaaaaaaaaaaaaaaaaaaaaaaaaaaaaaaaaa

SEQ ID NO: 172 - protein, 138 aa, Homo sapiens
MKFTTLLFLAAVAGALVYAEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 173 - DNA, 1223 bp, Homo sapiens
ccttgttccacgtagctggcaaggtcttcattcacttgccactgctagtcttccaacccttctggactttctttgctcttgtcttgttttgggtgtactggatcatgacacttctttttcttggcactaccggcagtcctgttcagaatgagcaaggctttgtggagttcaaaatttctgggcctctgcagtacatgtggtggtaccatgtggtgggcctgatttggatcagtgaatttattctagcatgtcagcagatgacagtggcaggagctgtggtaacatactattttactagggataaaaggaatttgccatttacacctattttggcatcagtaaatcgccttatycgttaccacctaggtacggtggcaaaaggatctttcattatcacattagtcaaaattccgcgaatgatccttatgtatattcacagtcagctcaaaggaaaggaaaatgcttgtgcacgatgtgtgctgaaatcttgcatttgttgcctttggtgtcttgaaaagtgcctaaattatttaaatcagaatgcatacacagccacagctatcaacagcaccaacttctgcacctcagcaaaggatgcctttgtcattctggtggagaatgctttgcgagtggctaccatcaacacagtaggagattttatgttattccttggcaaggtgctgatagtctgcagcacaggtttagctgggattatgctgctcaactaccagcaggactacacagtatgggtgctgcctctgatcatcgtctgcctctttgctttcctagtcgctcattgcttcctgtctatttatgaaatggtagtggatgtattattctkgkgttttgccattgawacaaaatacaatgatgggmgccctggcagagaattctatatggataaagtgctgatggagtttgtggaaaacagtaggaaagcaatgaaagaagctggtaagggaggcgtcgctgattccagagagctaaagccgatgctgaagaaaaggtgactggtctcatgagccctgaagaatgaactcagaggaggttgtttacatgaggttctcccactcaccagctgttgagagtctgcgattatgaagagcaggatcttattacttcaatgaaagcatgtaacaagtttctcaaaccaccaacagccaagtggatttggtacagtgcggctgtctaataaataatcaaaagcaaaaaaaaaaaaaaaa

SEQ ID NO: 174 - protein, 301 aa, Homo sapiens
  UNSURE (246)..(247)
  UNSURE (251)
  UNSURE (258)
MTLLFLGTTGSPVQNEQGFVEFKISGPLQYMWWYHVVGLIWISEFILACQQMTVAGAVVTYYFTRDKRNLPFTPILASVNRLIRYHLGTVAKGSFIITLVKIPRMILMYIHSQLKGKENACARCVLKSCICCLWCLEKCLNYLNQNAYTATAINSTNFCTSAKDAFVILVENALRVATINTVGDFMLFLGKVLIVCSTGLAGIMLLNYQQDYTVWVLPLIIVCLFAFLVAHCFLSIYEMVVDVLFXXFAIXTKYNDGXPGREFYMDKVLMEFVENSRKAMKEAGKGGVADSRELKPMLKKR

SEQ ID NO: 175 - DNA, 2460 bp, Homo sapiens
gagaagcgcgatgacggctacgaggccgctgccagctccaaaactagctcaggcgatgcctcctcactcagcatcgagggagactaacaaactccgggcaaagttggggctgaaacccttggaggttaatgccatcaagaaggaggcgggcaccaaggaggagcccgtgacagctgatgtcatcaaccctatggccttgcgacagcgagaggagctgcgggagaagctggcggctgccaaggagaagcgcctgctgaaccaaaagctggggaagataaagaccctaggagaggatgacccctggctggacgacactgcagcctggatcgagaggagccggcagctgcagaaggagaaggacctggcagagaagagggccaagttactggaggagatggaccaaaagtttggtgtcagcactctggtggaggaggagttcgggcagaggcggcaggacctgtacagtgcccgggacctgcagggcctcactgtggagcatgccattgattccttccgagaaggggagacaatgattcttaccctcaaggacaaaggcgtgctgcaggaggaggaggacgtgctggtgaacgtgaacctggtggataaggagcgggcagagaaaaatgtggagctgcggaagaagaagcctgactacctgccctatgccgaggacgagagcgtggacgacctggcgcagcaaaaacctcgctctatcctgtccaagtatgacgaaaagcttgaaggggagcggccacattccttccgcttggagcagggcggcacggctgatggcctgcgggagcgggagctggaggagatccgggccaagctgcggctgcaggctcagtccctgagcacagtggggccccggctggcctccgaatacctcacgcctgaggagatggtgacctttaaaaagaccaagcggagggtgaagaaaatccgcaagaaggagaaggaggtagtagtgcgggcagatgacttgctgcctctcggggaccagactcaggatggggactttggttccagactgcggggacggggtcgccgccgagtgtccgaagtggaggaggagaaggagcctgtgcctcagcccctgccgtcggacgacacccgagtggagaacatggacatcagtgatgaggaggaaggtggagctccaccgccggggtccccgcaggtgctggaggaggacgaggcggagctggagctgcagaagcagctggagaagggacgccggctgcgacagttacagcagctacagcagctgcgagacagtggcgagaaggtggtggagattgtgaagaagctggagtctcgccagcggggctgggaggaggatgaggatcccgagcggaagggggccatcgtgttcaacgccacgtccgagttctgccgcaccttgggggagatccccacctacgggctggctggcaatcgcgaggagcaggaggagctcatggactttgaacgggatgaggagcgctcagccaacggtggctccgaatctgacggggaggagaacatcggctggagcacggtgaacctggacgaggagaagcagcagcaggatgtgagggccacgccgctggggggtgggcgtttgggggtgctcaagctggagatgagcaccgggctcggtgtccagagcctcagcctcctcatccagagtgggctctgcagacctcccagggcgatctgaggagtaaatgaggaaattaaatgttgtggagggctggtgcctggcaggtggtgaccagtgggtggggctgagaagagccggtatggcctgctaaccacccccgccacgtgtcccgtagttctctgcttcctccaccaccatcctggacgaggaaccgatcgtgaatagggggctggcagctgccctgctcctgtgtcagaacaaagggctgctggagaccacagtgcagaaggtggcccgggtgaaggcccccaacaagtcgctgccctcagccgtgtactgcatcgaggataagatggccatcgatgacaagtacagccggagggaggaataccgaggcttcacacaggacttcaaggagaaggacggtacaaacccgacgttaagatcgaatacgtggatgagacgggccggaaactcacacccaaggaggctttccggcagctgtcgcaccgcttccatggcaagggctcaggcaagatgaagacagagcggcggatgaagaagctggacgaggaggcgctcctgaagaagatgagctccagsgacacgcccytgggcaccgtggccctgytccaggagaagcagaaggctcagaagaccccctacatygtgttcagcggcagcggcaagagcatgaacgcgaacaccatcaccaagtgacagcgccctcccgccccggccctgcctcaaccttcatattaaataaagctccctccttaaaaaaaaaaaaaaaaaaaaaaaaaaaaaaaaaaaaa

SEQ ID NO: 176 - protein, 563 aa, Homo sapiens
MTATRPLPAPKLAQAMPPHSASRETNKLRAKLGLKPLEVNAIKKEAGTKEEPVTADVINPMALRQREELREKLAAAKEKRLLNQKLGKIKTLGEDDPWLDDTAAWIERSRQLQKEKDLAEKRAKLLEEMDQKFGVSTLVEEEFGQRRQDLYSARDLQGLTVEHAIDSFREGETMILTLKDKGVLQEEEDVLVNVNLVDKERAEKNVELRKKKPDYLPYAEDESVDDLAQQKPRSILSKYDEKLEGERPHSFRLEQGGTADGLRERELEEIRAKLRLQAQSLSTVGPRLASEYLTPEEMVTFKKTKRRVKKIRKKEKEVVVRADDLLPLGDQTQDGDFGSRLRGRGRRRVSEVEEEKEPVPQPLPSDDTRVENMDISDEEEGGAPPPGSPQVLEEDEAELELQKQLEKGRRLRQLQQLQQLRDSGEKVVEIVKKLESRQRGWEEDEDPERKGAIVFNATSEFCRTLGEIPTYGLAGNREEQEELMDFERDEERSANGGSESDGEENIGWSTVNLDEEKQQQDVRATPLGGGRLGVLKLEMSTGLGVQSLSLLIQSGLCRPPRAI

SEQ ID NO: 177 - DNA, 1790 bp, Homo sapiens
ctggtacccacccctgtctctggacttctttatgaacactgagtatttgactccccacccttcatggtcagctccttttaacaccctagatatcattgctgtccttttcattgacatgctagagacatttccaattttcatgggagtcagtaagatagagatgatgaaaattcagagtgttcaaagaaagaggaggcctcatcagttgatagaatatacaacagattgtctaatctcagttttcttcacttttctacccattacagtggaaaaaagaacccttaaggaagccagtgcttctcaacattggttacaaaaccatcagtgccagcacttaatttaaaatcttctgaaaatccatcagttggttttcatgtctcactctaaactcagctccaaccaggctgcttcctagattcttgtctgtgtgtgtttctgttttggctctttgttacttccttctgtaaacttaagtaaactcagtttgttgacctagtgaattgattctttccattttctatactattcaccagcatatcaaagatgcttcttcctaactctgatttccagagtaactgtgctatcagcttttcatactaagaactaggtttttaccttttcacatttcttgcctcctacccagttcgtaagccaaattagactcgtactcttgtgaagtgccctttagatactagtgtaaaatatactgcgtaattctccattgccctacatatattaaccctttaagaaaatatatcaccagtctattttatcttcgcagaacttgcccaggtatgagccagcatttattaccactcattctagaaggtgccagttaggtagctatccaggtttacattttaaggcagtcgagaccttattcacgtggtatataaacaaccacatttctcttttatatggaacaagacttttcatttgcggctggccttctcactagcttatgctttttttttttaagacctttcttagcactcgctgaactcctccccctcacaatcaatctcagcaactcagcagagtcgcttcagttcacagctcttaatccttcactccagactaaagctaatcttatgtcctcaaacagctacaactcactgttatcacagttccgactgcagagactccatttaagaggaaatttaaaaaataaacaatgctctatttctgtccacatcaaaggtacctctaacagaaacttgtctttgcttctgtctctctgctactggactctcagctcaaggtaatgtaagatgatctcatagatgcactttagcctcttgatgttagcagttgagtcctccctgcgcacaaaatacatttgtggtaatgaatgcccagaatctcactggcacacccaagagtagttaaactaaaattgctactaacacaacattgtagaattcgttgccttaatggatgtgatcttctaggcttactgtgtcttaacaagcagtcttatatatatttatcttgcccagaaagtacacttgtagaatttaacatttgctcctttctccaaacagattaaaggttatgcagctgaatgattctgtgtcccattttaaaggtccaggctatcccctagatttagattagttcctaaaaaatcttccagtctgaatcctgacaaatggatagctaaattaatttccactcctcttctctcttgcactgtttggtacattacagccaccattagccttaaaagctgcagcagccaaattcggtgacagtaacataaagcctgagcttaaaaaaaaaaaaaaaaa

SEQ ID NO: 178 - protein, 115 aa, Homo sapiens [sequence omitted]

SEQ ID NO: 179 - DNA, 2026 bp, Homo sapiens
cattgagttgccctaacaacattgtctccagtgtcagaaccatattaaggttcgtttctcagattgggagcctgcaacaccatacagccaacattgcctttgccacgccactgccaccatccccaccattgccctatggtgggcagatgaattccagaaaccctcagggagccaggataattaggcaacccatctgaattggccacgtaagtgacaggcacttatctctcgggttcttgcttttgcagactccagggaagtcctgtctagaggtcgatggcagagactcctactctttcccatgaggggttgataggaatcaaattgggattcctttggctttgggttttgtttttttgttgttgtttttggttttcagtttgttttttggtgtatggggggtgattttgtttctgaataagaaaaagaagaggcaaccatggcccttatgtgggtttatcctttttgagcaatgttttagccacaagtaaggaatcttgaaagtcttttgtccagcaagcagtcttaaaaatgtttttcctaactccttttgcaggtgactaagtacaaaaaaatagttttctcattgtattcaaaatagtgagtaggttccctggataatacacagtggtagttgacatattttctcaaaacacaaccagaaaacccacttccggtatttgtaaatcacctttcaagggaaaaagtgaacacgtattccttgtatttctagtttgattaccaaacctgatgttacaaagaaacctccgttctgtagacagaatttcttttatttttcttcttttactcctcacaatcactttcccagtgccatcaccatctataaggtctcagagcagaggattattcatggtaataagtgggggtgtggtgcagccattccagtaacacccacaagaggacagctgttctgaatgtccccacccacccctctttcagtacaggtgagacattttcagttcatgagctccagaccaaatcccaggccagcccttgcaccaaaagccttttttagaaggcttatcagtctattaggaatgtctcaggaaagatgagccatttctttggggagaaatatatttacagatggaagtgtgtgactgcgtgtctgtgtgtgtgtgtggtgtgtgtgcgcacgtgagtgcgtgtgttcatctatgtgcatttcacttccataaagacccagcccaagctgctgggaaccatgtgttcctgagtattctcagaggttaaacaagtgacaagtgagcttctgaaattagtgtctcagcaagctggctttaggaatgagccccattttatcaagcagagaaaaaaaataacagcagaaaagataaagataaaccaaaaatatataccccccaatggaaaataatgttgattcagcaattcccataggatgtattacatgctctaatttattatattattatttatctgtctttgatctttgcccattgtactcttaaaaagatgttgggatgttgattgcgatttttaaacaactagataatgtataaatcagcagtggaaatcagttttaatgtgtggatgtgtctgattattgttaaatgcctctttttttactttttttttttttagatgtataatgtttcataaaccctggcactggtcacaaagctcagctgtgaaaatgaaatttgtagtatttttaaacatgaatgtcaatttcaagtgtatttgaaatggttcctccaggagagatatttgtgcaccattaggaaaatcttctctgcagaggaagtagccttctttggagaaaatggaaaatgggttctgatatgtgatctcagagtagcccatttcctagggcaccatggaaaacacaaatgtgatctttaagtatacctcttccccagtttggggaggaaaggactcagtttgcaccctttttgtatgtaaaataaaatgtcttacctttcttggctaaaaaaaaaaaaaaaaaaaaaaaaaaaaaaaaaaaaaaaaaaaaaaaaaaaaaaaa

SEQ ID NO: 180 - protein, 52 aa, Homo sapiens
MLQRNLRSVDRISFIFLLLLLTITFPVPSPSIRSQSRGLFMVISGGVVQPFQ

SEQ ID NO: 181 - DNA, 1138 bp, Homo sapiens
gggtctgtggagagccgggtgcgagcggcggcagcacgaggggaaaagagctgagcggagaccaaagtcagccgggagacagtgggtctgtgagagaccgaatagaggggctggggccacgagcgccattgacaagcaatggggaagaaacagaaaaacaagagcgaagacagcaccaaggatgacattgatcttgatgccttggctgcagaaatagaaggagctggtgctgccaaagaacaggagcctcaaaagtcaaaagggaaaaagaaaaaagagaaaaaaaagcaggactttgatgaagatgatatcctgaaagaactggaagaattgtctttggaagctcaaggcatcaaagctgacagagaaactgttgcagtgaagccaacagaaaacaatgaagaggaattcacctcaaaagataaaaaaaagaaaggacagaagggcaaaaaacagagttttgatgataatgatagcgaagaattggaagataaagattcaaaatcaaaaaagactgcaaaaccgaaagtggaaatgtactctgggagtgatgatgatgatgattttaacaaacttcctaaaaaagctaaagggaaagctcaaaaatcaaataagaagtgggatgggtcagaggaggatgaggataacagtaaaaaaattaaagagcgttcaagaataaattcttctggtgaaagtggtgatgaatcagatgaatttttgcaatctaaaaggacagaaaaaaaatcagaaaaacaagccaggtcctaacatagaaagtgggaatgaagatgatgacgcctccttcaaaattaagacagtggccccaaagaaggcagaaaagaaggagcgcgagagaaaaaagcgagatgaagaaaaagcgaaactgcggaagctgaaagaaaaagaagagttagaaacaggtaaaaaggatcagagtaaacaaaaggaatctcaaaggaaatttgaagaagaaactgtaaaatccaaagtgactgttgatactggagtaattcctgcctctgaagagaaagcagagactcccacagctgcagaagatgacaatgaaggagacaaaaagaagaaagataagaagaaaaagaaaggagaaaaggaagaaaaagagaaaaaaaaaaaaaaa

SEQ ID NO: 182 - protein, 209 aa, Homo sapiens
MGKKQKNKSEDSTKDDIDLDALAAEIEGAGAAKEQEPQKSKGKKKKEKKKQDFDEDDILKELEELSLEAQGIKADRETVAVKPTENNEEEFTSKDKKKKGQKGKKQSFDDNDSEELEDKDSKSKKTAKPKVEMYSGSDDDDDFNKLPKKAKGKAQKSNKKWDGSEEDEDNSKKIKERSRINSSGESGDESDEFLQSKRTEKKSEKQARS

SEQ ID NO: 183 - DNA, 912 bp, Homo sapiens
atccagctctttcagagagaagagatgggaataagataagaacatcatttcctgaagagtttactaagttgatgttttcttttggtgaagggcaatgtggaatttaaatcatatagttcatgtgtgcacctgtcctgcagtttgaggcagatgattttaaaccagcatctatagacacttcctgtgaaggagagcttcaagttggcaaaggagatgaagtcacaattacactgccacatatccctagctgagggcagcagtaaaatccaggcccgaatggaacagcagcccactcgtcctccacagacgtcacagccaccaccacctccaccacctatgccattcagagctccaacgaagcctccagttggacccaaaacttctcccttgaaagataacccgtcacctgaacctcagttggatgacatcaaaagagagctgagggctgaagttgacattattgaacaaatgagcagcagcagtgggagcagctcttcagactctgagagctcttcgggaagtgatgacgatagctccagcagtggaggsgaggamaatggcccagcytctcytccgcagcyttmacaccagcagccytacaacagtaggcctgccgttgccaatggaaccagccggccacaaggaagcaaccagytyatgaacaccctcagaaatgacttgcagttgagtgagtytggcagtgacagtgatgactagtgctggatytttcgaaacctactttttggtgcacaaacatgccgcaagactgagctactttggccgtggagtccattgcaagaggaaaatgttatggatcagtgactgtagtaggagtttgaggctytggaactctcacatattcaagtctttaacttagtggtgatgggtgaaaaaaaaaaaaaaaaaaaaaaa

SEQ ID NO: 184 - protein, 167 aa, Homo sapiens
  UNSURE (114)
  UNSURE (120)
  UNSURE (123)..(124)
  UNSURE (148)
  UNSURE (161)
MKSQLHCHISLAEGSSKIQARMEQQPTRPPQTSQPPPPPPPMPFRAPTKPPVGPKTSPLKDNPSPEPQLDDIKRELRAEVDIIEQMSSSSGSSSSDSESSSGSDDDSSSSGGEXNGPASXPQXXHQQPYNSRPAVANGTSRPQGSNQXMNTLRNDLQLSEXGSDSDD

SEQ ID NO: 185 - DNA, 4582 bp, Homo sapiens
ggtgaagctaatcaaatctggttcctatgtagctctcactgttcagggacgcccacctgggtcgccccagattccacttgccgactctgaagtagagccgtcagtcattggacatatgtctcccatcatgacatctcctcattcacctggagcatctgggaatatggagagaatcactagtcctgtgctcatgggggaggaaaacaatgtggttcataaccagaaagtagaaattctgagaaaaatgttacagaaagaacaggaacggctacagttattgcaggaagattacaaccgaacacctgcccaaagattgctaaaagagatccaagaggccaagaaacacattcctcagctgcaagagcagttatccaaagccacaggctctgctcaggatggagctgtagttacaccctccagacctttaggggacaccctaacagtcagtgaggcagaaacagatcctggagatgtactgggcaggactgactgtagcagtggagatgcttctcggcccagtagtgacaatgcagatagtcccaagagtggcccaaaagagagaatttatctagaggaaaacccagagaaaagtgaaacaattcaggacactgacactcaatcacttgtcggaagtccctcaacccgtatagcacctcatattattggagcagaagatgatgattttggtactgaacatgaacagatcaatggacagtgcagctgtttccagagcattgaattactaaaatctcgcccggctcatttggctgttttcttacaccatgtagtttcacaatttgaccctgcgactttgctctgttatctctattcagacctgtataaacataccaattccaaagaaactcgtcgcatcttccttgagtttcatcagttctttctaaatcgatcagcacacctgaaagtttctgttcctgatgaaatgtctgcagatctagaaaagagaagacctgagctcattcctgaggatctgcatcgccactatatccaaactatgcaagaaagagtccatccagaagttcaaaggcacttaaaagattttcggcagaaacgtagtatgggactgaccttggctgaaagcgagctgactaaacttgatgcagagcgagacaaggaccgattgactttggagaaggagcggacatgtgcagaacagattgttgccaaaattgaagaagtattgatgaytgctcaggctgtaraggaagataagagctccaccatgcagtatgttattctcatgtatatgaagcatttgggagtaaaagtgaaagagcctcgaaatttggagcacaaacggggtcggattggatttcttcccaaaatcaagcaaagtatgaagaaagataaagaaggggaagaaaaagggaagcgaagaggattccccagcatcctgggacccccacggagaccaagccgtcatgacaacagtgcaattggcagagccatggaactacagaaggcgcgccaccctaagcacttatccacaccctcatctgtgagtcctgaacctcaggactctgccaagttgcgccagagtgggttagcaaatgaaggaacagacgctggatacctgcctgccaattccatgtcttctgtagcttcaggggcctctttttcccaggaaggagggaaagagaatgatacaggatcaaagcaagttggagaaacatcagcacctggagacaccttagatggcacacctcgtactctcaatactgtctttgatttcccaccacctccattagaccaagtgcaggaggaggaatgtgaagtagaaagggtgactgaacatgggacaccaaagccctttcgaaagtttgacagtgtagcttttggagaaagtcaaagtgaggatgaacaatttgaaaatgacttagagacagatccacccaactggcagcagcttgttagtcgagaagtgttactgggactaaaaccttgtgaaatcaaaagacaggaagtgattaatgaattgttctacactgaaagagctcatgttcgaacactgaaggttcttgatcaagtgttctatcagcgagtatccagagaaggaattctgtcaccctcagagctacggaaaattttttcaaacttggaagatattcttcaacttcatattggattgaatgaacaaatgaaggctgttcgaaagagaaatgagacctctgttatcgatcagattggggaagatttgctgacatggttcagcggaccaggagaggagaaattgaaacatgctgctgctaccttttgcagtaaccaacctttcgccctggaaatgatcaaatctcgtcagaaaaaggattctcgatttcagacttttgtgcaagatgctgaaagtaatccactgtgtcgtcgtcttcaactgaaggatattattcccactcaaatgcaaaggcttactaagtacccacttctgttggataatattgccaaatacacagaatggccaacagaaagggagaaggtgaagaaagctgcagatcactgtcgtcagatcttaaattatgtaaatcaggctgtcaaggaggcagaaaacaagcagcgcctagaagattatcagcgtcgccttgatacctccagcctgaagttgtcagagtacccaaatgttgaagagctcaggaatttggatttaacaaaaaggaagatgattcatgaagggccattggtttggaaggtgaatagagataaaactattgatttatacacgttgctgctggaagacattcttgtattgttacaaaagcaggatgatagactggttttaaggtgtcatagtaagattctggcatctacagctgatagcaaacacacgtttagccctgtcattaagttgagtacagtgttggttcgacaagtggcaacagataacaaagctttattcgtcatttccatgtcagacaatggcgctcagatttatgaactggtggcacagacagtttctgaaaagactgtctggcaggacctaatctgtcggatggctgcatcagtgaaggagcaatccacaaagccgattccattaccacagtcaacacctggcgaaggagataatgatgaagaagatccttcaaaattaaaagaggagcagcatggcatttcagtcactggtttgcagagtccagacagagatttgggattagaatctaccttaatatcgtcaaaacctcagtctcattcactgagtacctctgggaaatcagaggtacgtgatctgtttgtggctgagagacagtttgcaaaggaacaacatacagatgggacactaaaggaagttggagaagattatcaaatcgcaatcccagattcacacctgcctgtctcagaagaacggtgggcattggatgcactaagaaatttgggtttgttgaagcagttgctggtgcaacagctaggtttgactgagaagagcgttcaggaagactggcaacatttcccaagatacagaacagcctctcaggggccgcagacagacagtgtcatccagaactctgaaaatattaaggcctatcattctggtgaaggacatatgccctttagaactggaactggtgacattgcaacttgttacagtccacggacttcaactgaatcttttgctccacgggattcagtgggactggcaccccaggatagccaggcaagtaacattttagtaatggaccacatgattatgaccccagagatgcctaccatggagccagaagggggtcttgatgacagtggagagcacttttttgatgcccgtgaagcacatagtgatgagaatccatcagaaggtgatggagcagttaacaaggaagagaaggatgttaatttacgcatctcaggaaactatttgatccttgatggctatgacccagtgcaggagagttccacagatgaggaggttgcttcctcacttaccctgcagcccatgacaggcatccctgctgtggaatccacccaccagcagcaacattctcctcagaatactcactccgatggggcaatttcaccattcacccccgaatttctggtccagcagcgctggggagctatggagtattcctgttttgagatccagagtccctcctcttgtgcagattcacagagccagatcatggagtacattcataagatagaggctgaccttgaacacttaaaggaaggtggaggaaagttaacaccattctttgccaaaggctggctggatcagccctcacagacaagcactcagataaaagttagagccgcatgtcctggaggtgactgcaggttgttggatttggagtatcggccgtgtctcaccacatcctggctccagtgtggatgcagagagagtgtgacagaggatctgcctgtgaaccacctgggattagtcaagtcccaaggtgcccagagtgggactagttyttcacagtgtggcagctgcactaatctgtttgtgagggaatatccattccctcactctactctcctcactatcggaaattcattttgattcagaataaaaaccaaatgtatagagctttgggttgtaggatatgaaattgtacttagatttaagaaaaaaaaaaaaaaa

SEQ ID NO: 186 - protein, 1461 aa, Homo sapiens
  UNSURE (364)
  UNSURE (369)
  UNSURE (1433)
MSPIMTSPHSPGASGNMERITSPVLMGEENNVVHNQKVEILRKMLQKEQERLQLLQEDYNRTPAQRLLKEIQEAKKHIPQLQEQLSKATGSAQDGAVVTPSRPLGDTLTVSEAETDPGDVLGRTDCSSGDASRPSSDNADSPKSGPKERIYLEENPEKSETIQDTDTQSLVGSPSTRIAPHIIGAEDDDFGTEHEQINGQCSCFQSIELLKSRPAHLAVFLHHVVSQFDPATLLCYLYSDLYKHTNSKETRRIFLEFHQFFLNRSAHLKVSVPDEMSADLEKRRPELIPEDLHRHYIQTMQERVHPEVQRHLKDFRQKRSMGLTLAESELTKLDAERDKDRLTLEKERTCAEQIVAKIEEVLMXAQAVXEDKSSTMQYVILMYMKHLGVKVKEPRNLEHKRGRIGFLPKIKQSMKKDKEGEEKGKRRGFPSILGPPRRPSRHDNSAIGRAMELQKARHPKHLSTPSSVSPEPQDSAKLRQSGLANEGTDAGYLPANSMSSVASGASFSQEGGKENDTGSKQVGETSAPGDTLDGTPRTLNTVFDFPPPPLDQVQEEECEVERVTEHGTPKPFRKFDSVAFGESQSEDEQFENDLETDPPNWQQLVSREVLLGLKPCEIKRQEVINELFYTERAHVRTLKVLDQVFYQRVSREGILSPSELRKIFSNLEDILQLHIGLNEQMKAVRKRNETSVIDQIGEDLLTWFSGPGEEKLKHAAATFCSNQPFALEMIKSRQKKDSRFQTFVQDAESNPLCRRLQLKDIIPTQMQRLTKYPLLLDNIAKYTEWPTEREKVKKAADHCRQILNYVNQAVKEAENKQRLEDYQRRLDTSSLKLSEYPNVEELRNLDLTKRKMIHEGPLVWKVNRDKTIDLYTLLLEDILVLLQKQDDRLVLRCHSKILASTADSKHTFSPVIKLSTVLVRQVATDNKALFVISMSDNGAQIYELVAQTVSEKTVWQDLICRMAASVKEQSTKPIPLPQSTPGEGDNDEEDPSKLKEEQHGISVTGLQSPDRDLGLESTLISSKPQSHSLSTSGKSEVRDLFVAERQFAKEQHTDGTLKEVGEDYQIAIPDSHLPVSEERWALDALRNLGLLKQLLVQQLGLTEKSVQEDWQHFPRYRTASQGPQTDSVIQNSENIKAYHSGEGHMPFRTGTGDIATCYSPRTSTESFAPRDSVGLAPQDSQASNILVMDHMIMTPEMPTMEPEGGLDDSGEHFFDAREAHSDENPSEGDGAVNKEEKDVNLRISGNYLILDGYDPVQESSTDEEVASSLTLQPMTGIPAVESTHQQQHSPQNTHSDGAISPFTPEFLVQQRWGAMEYSCFEIQSPSSCADSQSQIMEYIHKIEADLEHLKEGGGKLTPFFAKGWLDQPSQTSTQIKVRAACPGGDCRLLDLEYRPCLTTSWLQCGCRESVTEDLPVNHLGLVKSQGAQSGTSXSQCGSCTNLFVREYPFPHSTLLTIGNSF

SEQ ID NO: 187 - DNA, 2837 bp, Homo sapiens
tcttttgacagatataattgctgcttatcaaagattctgttctcgacccccaaaaggatttgaaaaatactttcctaatggaaaaaatggaaaaaaagctagtgaacctaaagaagttatgggagagaaaaaagaatcaaagccagctgctaccacacgctcttctggaggaggaggtggtggcggtggaaaacgaggtggcaagaaagatgattctcactggtggtccaggtttcagaagggtgacattccatgggacgacaaggatttcaggatgttcttcctctggactgctctgttctggggtggagtcatgttttacttgctgctcaagagatccgggagagaaatcacttggaaggactttgtcaataactatctttcaaaaggagtagtagacagattggaagtcgtcaacaagcgttttgttcgagtgacctttacaccaggaaaaactcctgttgatgggcaatacgtttggtttaatattggcagtgtggacacctttgaacggaatctggaaactttacagcaggaattgggcatagaaggagaaaatcgggtgcctgttgtctacattgctgaaagtgatggctcttttctgctgagcatgctgcctacggtgctcatcatcgccttcttgctctacaccatcagaagagggcctgctggcattggccggacaggccgagggatgggcggactcttcagtgtcggagaaaccactgccaaggtcttaaaggatgaaattgatgtgaagttcaaagatgtggctggctgtgaggaggccaagctagagatcatggaatttgtgaatttcttgaaaaacccaaagcagtatcaagacctaggagcaataatcccaaagggtgccattctcactggtcctccaggcactgggaagacgctgctagctaaggccacagccggagaagccaatgtccccttcatcaccgttagtggatctgagtttttggagatgttcgttggtgtgggccctgctagagtccgagacttatttgcccttgctcggaagaatgccccttgcatcctcttcatcgatgaaatcgatgcggtgggaaggaagagaggaagaggcaactttggagggcagagtgagcaggagaacacactcaaccagctgctggtggagatggatggttttaatacaacaacaaatgtcgtcattttggccggcaccaatcgaccaggaccaccagacataaaaggaagagcttctattttcaaagttcatctccgaccgctaaaactggacagtaccctggagaaggataaattggcaagaaaactggcatctttaactccagggttttcaggtgctgatgttgctaatgtctgtaatgaagctgcgttgattgctgcaaggcatctgtcagattccataaatcagaaacactttgaacaggcaattgagcgagtgattggtggcttaaagaaaaaaacgcaggttctgcagcctgaggagaaaaagactgtggcataccacgaagcaggccatgcggttgccggctggtatctggagcacgcagacccgcttttaaaggtatccatcatcccacgtggcaaaggactaggttatgctcagtatttaccaaaagaacaatacctctataccaaagagcagctcttggataggatgtgtatgactttaggtggtcgagtctctgaagaaatcttctttggaagaattacaactggtgctcaagatgacttgagaaaagtaactcagagtgcatatgcccaaattgttcagtttggcatgaatgaaaaggttgggcaaatctcctttgacctcccacgtcagggggacatggtattggagaaaccttacagtgaagccactgcaagattgatagatgatgaagtacgaatacttattaatgatgcttataaaagaacagtagctcttctcacagaaaagaaagctgacgtggagaaggttgctcttctgttgttagaaaaagaagtattagataagaatgatatggttgaacttttgggccccagaccatttgcggaaaaatctacctatgaagaatttgtggaaggcactggcagcttggatgaggacacctcacttccagaaggccttaaggactggaacaaggagcgggaaaaggagaaagaggagcccccgggtgagaaagttgccaactagaggcccagagggaggccatctcagtctgtccactgtggtttcagctggtgcattatttcagctgtggctttcagaagaatgggaatgctgcgctgattttagccagccactggcccagctgaaatgatggggaaaggagtccttagtcctttcagcctcagaggtcacagtgggtggcaggtgactttccggaggccttgagggaaatgcacactgtcccatagcctcattgggttcccagacgtgctggaaaggttgagcccagagtggccgaggctggaccctgtggcaccaagtggggtcggctgaccgtgtggcagggattgttgcamtggawttttggcgtgtgggaagggatgctttttttttgtcgcccayttttcattcctgtttttcctcagttcccckgkgcagatgggctgtgaaattaaattggagttttgataagaacattttaatttgacttaatattttaaagattgaatccagatcacttgttgctgttttaatggaatggttttttacaggagctgtaacatamttaaaaatatgaatgtattatgtaaatatggcttctttacataaaaaataaaatgtcaacactgtaaaaaaaaaaaaaaaaaaaaaaaaaaaaaaaaaaaaaaaaaaaaaaaaaaaa

SEQ ID NO: 188 - protein, 686 aa, Homo sapiens
MGEKKESKPAATTRSSGGGGGGGGKRGGKKDDSHWWSRFQKGDIPWDDKDFRMFFLWTALFWGGVMFYLLLKRSGREITWKDFVNNYLSKGVVDRLEVVNKRFVRVTFTPGKTPVDGQYVWFNIGSVDTFERNLETLQQELGIEGENRVPVVYIAESDGSFLLSMLPTVLIIAFLLYTIRRGPAGIGRTGRGMGGLFSVGETTAKVLKDEIDVKFKDVAGCEEAKLEIMEFVNFLKNPKQYQDLGAIIPKGAILTGPPGTGKTLLAKATAGEANVPFITVSGSEFLEMFVGVGPARVRDLFALARKNAPCILFIDEIDAVGRKRGRGNFGGQSEQENTLNQLLVEMDGFNTTTNVVILAGTNRPGPPDIKGRASIFKVHLRPLKLDSTLEKDKLARKLASLTPGFSGADVANVCNEAALIAARHLSDSINQKHFEQAIERVIGGLKKKTQVLQPEEKKTVAYHEAGHAVAGWYLEHADPLLKVSIIPRGKGLGYAQYLPKEQYLYTKEQLLDRMCMTLGGRVSEEIFFGRITTGAQDDLRKVTQSAYAQIVQFGMNEKVGQISFDLPRQGDMVLEKPYSEATARLIDDEVRILINDAYKRTVALLTEKKADVEKVALLLLEKEVLDKNDMVELLGPRPFAEKSTYEEFVEGTGSLDEDTSLPEGLKDWNKEREKEKEEPPGEKVAN

SEQ ID NO: 189 - DNA, 627 bp, Homo sapiens
gattggagttcttttcttaaatatccaaataagattatagctatgtttttaccaaatgtttgtgcggtaaatagagagtggatggaaattaaccctagaaaggatagttgtaacttttaaaaagttgattaactatttcgtgtgctaatttgagtttttctgaatactccaatatggtttcctttaacacctgctctcagtttacaatcacctaacttcccagcgttggtgtctttttctctgtctgaccctgtcttatttctcctacaaagacatatcctgcgctgtacttcagatacttttttcgaggaacatttgtgatttgtggcataaagtaactgtctaaaggaaatcttctgagaggatctggtcattttatgaaaggggcaattaaggggaaatggaagcagatcttttaaagaaggagcatttgaaattagcccaggaatcatgtccggcgagtcctgctcttttgtacctgggcataatagtcagccacacagagctagagttagttcaagaattgtctttcctgatcgtgctatatttttggaaacacgttagatacagaggtaagatgtcaaaattctgaaatacacacaatataggatcaaaaaaaaaaaaaaa

SEQ ID NO: 190 - protein, 63 aa, Homo sapiens [sequence omitted]

SEQ ID NO: 191 - DNA, 868 bp, Homo sapiens
  unsure (733)
attctgtccctgcccttagaaaacctaaaataccaagggtgtcatgctggcaactccctgcccagtcctgcacaaagccttggctgtgtgtggcaccccttgcctcctaccccagagcagctggctccattggcttctccctgcaccagccctgtcctcaggggtcaggaaaaagcacacagctttctttcctctcctccagaggcctggaagggaggtggaggtccagtaagggcctggctgccttggatttcttggtcctgccttgccaactgcaccctgtagctcctgctccctgtgaccccagaacagaggtgctgccttccctgtctcctagacaaagcacaaagggatgccctgcttggcttgagcctgcccaactgaaggattttctctgccccagggaccttccatccctgaatacaaggctctaggcaacttctctctgggtggtacacactagaatgcctggcattagccctagaaaggaggttggggtgtatgggtagtgagctagggtgggagaaaggtggtgctgaaaggacagatgctagttgtagtttcactcactcattcattcattagtgcaacagtactgagcaccacctgcactagaggcagaggggtgaacaagatacccttttgcctggggggacgtccacttcccatgggtttggctatttccaggaaagcccctcagtcctcctccctgttctggctgtgtgtgaaggangtgtgtgagcaggcccaatcctttgcagcaagaatgagaggtcagagtattccattgcacacgcaccctggggctgacagacttgtgccccctagccttcatgcatgcccaagcactggcagctttgcagcccct

SEQ ID NO: 192 - protein, 107 aa, Homo sapiens
  UNSURE (62)
MLVVVSLTHSFISATVLSTTCTRGRGVNKIPFCLGGRPLPMGLAISRKAPQSSSLFWLCVKXVCEQAQSFAARMRGQSIPLHTHPGADRLVPPSLHACPSTGSFAAP

SEQ ID NO: 193 - DNA, 467 bp, Homo sapiens
  unsure (57)
  unsure (254)
tggctatccctagaagcacttcatccatcttaacccacccaaacgggatcccttcanatctcatacccagtaagatgcaagaaaggaatatttgagagcaagcagccctgttccaggggccccaggtatgtgtagaggcccagtgggggtggccacttggtgtttctaccaccccctgccatccagtctggcccagtacctacctgggaggttggtgtacttggcttaagtacttcatgctttattcaggctgnttccccacagcaccggcaggaaatgaaggtgcacttatatgcatccctgcaggaataaagagtgggtggcctgcccagcccagcaccacagcctttccccagccaggagagaccacctaaggatcaaggcagctcctgttttcttggttctgtgacactcgagtctgagccagcccctcaggaattgcctcaaaagagaaaaaaaaaaaaaaa

SEQ ID NO: 194 - DNA, 1035 bp, Homo sapiens
aatctttttgttgtcaagcttgaggtgtggcaggcttgagatctggccatacacttgagtgacaatgacatccactttgcctttctctccacaggtgtccactcccaggtccaactgcagacttcgaattcggccttcatggcctagatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtrcagcgagcagtataccagcagcagcaagcgaaaggaagagtttgctgatatgtcaaaagtttcattcagtgggaagcaatgggcttctggactttgattcagaatatcmggagctctgggattggctgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactrcttggggtatttgcatttttattactgtttgtaggttatgtgtacattttttgcgtagtgaagtactctgtccgatttctaatttgaggcacaaatatctctctctttcaattcactacctacgtttcaaacaagctattcatgctattatgggaaagacactgcttttcctcttctgttgattttttttttttctgagcttgtcccctctcagattttaataattttggttctttaatacatgaaaaagtaagtaaaatatgccatgtattatgggtatgcaccaagtcaactataatacagtatatctgatatataaaaaaaaaaaaaaa

SEQ ID NO: 195 - protein, 179 aa, Homo sapiens
  UNSURE (37)
  UNSURE (73)
MCLWRMRKGLEAPRLRFNYATWKHKEMLLSRCPSSCXASSIPAAASERKSLLICQKFHSVGSNGLLDFDSEYXELWDWLIDMESLVMDSHDLMMSEEQQQHLYKRYSVEMSIRHLKKTELLSKVEALKKGGVLLPNDLLEKVDSINEKWELLGVFAFLLLFVGYVYIFCVVKYSVRFLI

SEQ ID NO: 196 - DNA, 3831 bp, Homo sapiens
gcagcaggtcaaacaaggcatctcctagtattgcatcctacagatgtgctgtaaacatcaaaagaagacggtgggatcaggagatgctgttttggaaagaagtgaggtttagacttctccatgttaaccatgagcgtgacactttcccccctgaggtcacaggacctggatcccatggctactgatgcttcacccatggccatcaacatgacacccactgtggagcagggtgagggagaagaggcaatgaaggacatggactctgaccagcagtatgaaaagccacccccactacacacaggggctgactggaagattgtcctccacttacctgaaattgagacctggctccggatgacctcagagagggtccgagacctaacctattcagtccagcaggattcggacagcaagcatgtggatgtacatctagttcaactaaaggacatttgtgaagatatttctgatcatgttgagcaaatccatgccctccttgaaacagagttctccctaaagctgctgtcttactctgtcaacgtgatagtggacatccacgcagtgcagctcctctggcaccagcttcgagtctcagtgctggttctgcgggagcgcattctgcaaggtctgcaggacgccaatggcaactacactaggcagacggacattctgcaagctttctctgaagagacaaaagagggccggcttgattctctaacagaagtggatgactcaggacaattaaccatcaaatgttctcaaaattacttgtctctggattgtggcattactgcattcgaactgtctgactacagtccaagtgaggatttgctcagtgggctaggtgacatgacctctagccaagtcaaaaccaaaccctttgactcttggagctacagtgagatggaaaaggagtttcctgagcttatccgaagtgttggtttacttacggtagctgctgactctatctctaccaatggcagtgaagcagttactgaggaggtatctcaagtatctctctcagtagacgacaaaggtggatgtgaggaagacaatgcttctgcagtcgaagagcaaccaggcttaacactgggggtgtcatcatcttcaggagaagctctgacaaatgctgctcaaccctcctctgagactgtgcagcaagaatccagttcctcctcccatcatgatgcaaagaatcagcagcctgttccttgtgaaaatgcaacccccaaacgaaccatcagagattgctttaattataacgaggactctcccacacagcctacattgccaaaaagaggactttttcttaaagaggaaacttttaagaatgatctgaaaggcaatggtggaaagaggcaaatggttgatctaaagcctgagatgagcagaagcaccccttcgctagtagatcctcctgacagatccaaactttgcctggtattgcagtcttcttaccccaacagcccttctgctgccagccagtcttatgagtgtttacacaaggtggggaatgggaaccttgaaaacacagtcaaatttcacattaaagaaatttcttccagcctgggaaggcttaacgactgctataaagagaaatctcgacttaaaaagccacacaagacctcagaagaggtgcctccatgccgaacacctaaacgggggactggttcaggcaaacaagctaaaaatacaaagagctcagcagtgccaaatggagagctttcttatacttccaaggccatagaggggccacaaacaaattctgcttccacatcctcacttgagccttgtaatcagagaagttggaatgccaaattgcaattgcagtcagaaacatccagttcaccagcttttactcagagcagtgaatcctctgttggctcagacaacatcatgtctccggtgccacttctttcaaaacacaaaagcaaaaaaggtcaagcctcctctccaagtcacgtcactaggaatggtgaggttgtggaggcctggtatggctctgatgaatacctagcactgccctctcaccttaagcagacagaagtattggctttgaagttggaaaacctaacaaagcttctgcctcagaaacccagaggagaaaccatccagaatattgatgactgggaactgtctgaaatgaattcagattctgaaatctatccaacctatcatgtcaaaaagaagcatacaaggctaggcagggtgtctccaagctcatctagtgacatagcctcttcactaggggagagcattgaatctgggcccctgagtgacattctttctgatgaggagtccagtatgcctctcgctggcatgaaaaagtatgctgatgagaagtcagaaagagcttcatcctctgagaaaaatgagagccattctgccactaaatcagctttaattcagaaactgatgcaagatattcagcaccaagacaactatgaagccatatgggaaaaaatagaggggtttgtaaacaaactggatgaattcattcaatggttaaatgaagccatggaaactacagagaattggactccccctaaagcagagatggatgaccttaaactgtatctggagacacacttgagttttaagttgaatgtagacagtcattgtgctctcaaggaagctgtggaggaggaaggacaccaacttcttgagcttattgcatctcacaaagcaggactgaaggacatgctgcggatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtacagcgagcagtataccagcagcagcaagcgaaaggaagagtttgctgatatgtcaaaagttcattcagtgggaagcaatgggcttctggactttgattcagaatatcaggagctctgggattggctgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactgcttggggtatttgcatttttattactgtttgtaggttatgtgtacattttttgcgtagtgaagtactctgtccgatttctaatttgaggcacaaatatctctctctttcaattcactacctacgtttcaaacaagctattcatgctattatgggaaagacactgcttttcctcttctgttgattttttttttttctgagcttgtcccctctcagattttaataattttggttctttaatacatgaaaaagtaagtaaaatatgccatgtattatgggtatgcaccaagtcaactataatacagtatatctgatatatactgactgtcatgcttgaatgaatgttaatagaatttattctgaaggtacatgtgagagacatctactgtttaactatttactgtacccttaagatgaaaagtggagttgtcactacagctttcaagtcacactaaagccaccaaaacaaagatgcaaatttgacccaaatctgaattgcagaattgaatcagcctgtgttttgtgcctcaatttccagctcacttttaacaaaagccaaaaaaaaaaaaaaa

SEQ ID NO: 197 - protein, 1075 aa, Homo sapiens
MLTMSVTLSPLRSQDLDPMATDASPMAINMTPTVEQGEGEEAMKDMDSDQQYEKPPPLHTGADWKIVLHLPEIETWLRMTSERVRDLTYSVQQDSDSKHVDVHLVQLKDICEDISDHVEQIHALLETEFSLKLLSYSVNVIVDIHAVQLLWHQLRVSVLVLRERILQGLQDANGNYTRQTDILQAFSEETKEGRLDSLTEVDDSGQLTIKCSQNYLSLDCGITAFELSDYSPSEDLLSGLGDMTSSQVKTKPFDSWSYSEMEKEFPELIRSVGLLTVAADSISTNGSEAVTEEVSQVSLSVDDKGGCEEDNASAVEEQPGLTLGVSSSSGEALTNAAQPSSETVQQESSSSSHHDAKNQQPVPCENATPKRTIRDCFNYNEDSPTQPTLPKRGLFLKEETFKNDLKGNGGKRQMVDLKPEMSRSTPSLVDPPDRSKLCLVLQSSYPNSPSAASQSYECLHKVGNGNLENTVKFHIKEISSSLGRLNDCYKEKSRLKKPHKTSEEVPPCRTPKRGTGSGKQAKNTKSSAVPNGELSYTSKAIEGPQTNSASTSSLEPCNQRSWNAKLQLQSETSSSPAFTQSSESSVGSDNIMSPVPLLSKHKSKKGQASSPSHVTRNGEVVEAWYGSDEYLALPSHLKQTEVLALKLENLTKLLPQKPRGETIQNIDDWELSEMNSDSEIYPTYHVKKKHTRLGRVSPSSSSDIASSLGESIESGPLSDILSDEESSMPLAGMKKYADEKSERASSSEKNESHSATKSALIQKLMQDIQHQDNYEAIWEKIEGFVNKLDEFIQWLNEAMETTENWTPPKAEMDDLKLYLETHLSFKLNVDSHCALKEAVEEEGHQLLELIASHKAGLKDMLRMIASQWKELQRQIKRQHSWILRALDTIKAEILATDVSVEDEEGTGSPKAEVQLCYLEAQRDAVEQMSLKLYSEQYTSSSKRKEEFADMSKVHSVGSNGLLDFDSEYQELWDWLIDMESLVMDSHDLMMSEEQQQHLYKRYSVEMSIRHLKKTELLSKVEALKKGGVLLPNDLLEKVDSINEKWELLGVFAFLLLFVGYVYIFCVVKYSVRFLI

SEQ ID NO: 198 - DNA, 3113 bp, Homo sapiens
gcaaacacattgaagtacaggtagcccaggaaactagaaatgtatctactggctctgctgaaaatgaagaaaagtcagaagttcaagcaatcatcgaatctactcctgagctggatatggacaaagatctcagtggatataaaggttcaagcactcccaccaaaggcatagagaacaaagcttttgatcgcaatacagaatctctctttgaagaactgtcttcagctggctcaggcctaataggagatgtggatgaaggagcagatttactaggaatgggtcgggaagttgagaatcttatattagaaaatacacaactgttggaaaccaaaaatgctttgaacatagtgaagaatgatttgatagcaaaagtggatgaactgacctgtgagaaagatgtgctgcaaggggaattggaggctgtgaagcaagccaaactgaaactagaggaaaagaacagagaattggaggaagagcttaggaaagctcgggcagaagctgaagatgcagggcaaaaagcaaaagatgacgatgatagtgatattcccacagcccagaggaaacggtttactagagtagaaatggcccgtgttctcatggagcgaaaccagtataaagagagattgatggagcttcaggaagctgttcgatggacagagatgattcgggcatcacgagaaaatccagccatgcaggaaaaaaaaaggtcaagcatttggcagtttttcagccgacttttcagctcctcaagtaacacgactaagaagcctgaaccacctgttaatctgaagtacaatgcacccacgtctcatgttactccgtccgtcaagaaaagaagcagcaccttatctcagctccctggggataagtccaaagcctttgatttccttagtgaagaaactgaagctagtttagcctcacgcagagaacaaaagagagagcagtatcgtcaggtaaaagcacatgttcagaaggaagacggtagagtgcaggcttttggctggagtctgcctcagaagtacaaacaggtaaccaatggtcaaggtgaaaataagatgaaaaatttacctgtgcctgtctatctcagacctctggatgaaaaagatacatcaatgaagctgtggtgtgctgttggagtcaatttatctggtgggaagaccagagatggtggttctgttgttggagcaagtgtattttacaaggatgttgctggtttggatacagaaggcagtaaacagcgaagtgcctctcagagtagtttagataagttagatcaggaacttaaggaacagcagaaggagttaaaaaatcaagaagaattatccagtctagtttggatctgtaccagcactcattcggctacaaaagttcttattattgatgctgttcaacctggcaacatcctagacagtttcactgtttgcaactctcatgttctgtgcattgcaagtgtgccaggtgcacgagaaacagactaccctgcaggagaagatctttcagaatctggtcaggtagacaaagcatctttatgtggaagtatgacaagcaacagctcagcagagacagacagcctgttaggaggcatcacagtggttggttgttctgcagaaggtgtgacgggagctgccacttcccctagtacaaatggtgcttctccagtgatggataaaccaccagaaatggaagcagaaaatagtgaggttgatgaaaatgttccaacagcagaagaagcaactgaagctacagaagggaatgcggggtcagctgaagacacagtggacatctcccaaactggcgtctacacagagcatgtctttacagatcctttgggagttcagatcccagaagacctctccccagtgtatcagtcgagcaatgactcagatgcatataaagatcaaatatcagtactgccaaatgaacaagacttggtgagagaagaagcccagaaaatgagtagtcttttaccaactatgtggcttggagctcaaaatggctgtttgtatgtccattcatctgtagcccagtggaggaaatgtctccattccattaaacttaaagattcgattctcagtattgtacacgtgaagggaatcgtgttagtagccctggctgacggcacccttgcaatctttcacagaggagtggatgggcagtgggatttgtcaaactatcacctcttagaccttggacggcctcatcattccatccgttgcatgactgtggtacatgacaaagtctggtgtggctataggaacaaaatctatgtggtgcagccaaaggccatgaaaatagagaaatcttttgatgcacatcccaggaaggagagccaagtgcgacagcttgcgtgggtgggggatggcgtgtgggtctccattcgcttggattctacgctccgtctctatcatgcacacacttatcaacatctacaggatgtggacattgagccttatgtaagcaaaatgttaggtactggaaaactgggcttctcttttgtgagaattacagctcttatggtgtcttgtaatcgtttgtgggtggggacaggaaatggtgtcattatctccatcccattgacagaaagtaagtatatttttagataactgccatggaacaaataggagaattataggaaatagttatgtctgaaattcaaatctgtgttagctcaagaaaatcagtgatggcaaagatctagctatagttaacttcaactcaatgaaacttataaattctaggaaaataagttttcatgtatcaagaccaatactcataattaaaagtttttcgttgtcaaaacattttcacagttcaggaatcacagtcctatttcatcttttgaaataaaagttaaagtccatttttcttaagccttattataccattatccaaaaacactacctagcattcatcttagtttttcatatctccttagcagatagtttagtatattccatttgtcttatttgaggtttaaaaaaagttgtggaagaagtttctcaggagttcagcaagcctttccatctatcttctattttaaaagaatgtgatttattgtttttatgaataaaatatacctcgtgtgtttctcaaaaaaaaaaaaaaaaaaaaaa

SEQ ID NO: 199 - protein, 828 aa, Homo sapiens
MDKDLSGYKGSSTPTKGIENKAFDRNTESLFEELSSAGSGLIGDVDEGADLLGMGREVENLILENTQLLETKNALNIVKNDLIAKVDELTCEKDVLQGELEAVKQAKLKLEEKNRELEEELRKARAEAEDAGQKAKDDDDSDIPTAQRKRFTRVEMARVLMERNQYKERLMELQEAVRWTEMIRASRENPAMQEKKRSSIWQFFSRLFSSSSNTTKKPEPPVNLKYNAPTSHVTPSVKKRSSTLSQLPGDKSKAFDFLSEETEASLASRREQKREQYRQVKAHVQKEDGRVQAFGWSLPQKYKQVTNGQGENKMKNLPVPVYLRPLDEKDTSMKLWCAVGVNLSGGKTRDGGSVVGASVFYKDVAGLDTEGSKQRSASQSSLDKLDQELKEQQKELKNQEELSSLVWICTSTHSATKVLIIDAVQPGNILDSFTVCNSHVLCIASVPGARETDYPAGEDLSESGQVDKASLCGSMTSNSSAETDSLLGGITVVGCSAEGVTGAATSPSTNGASPVMDKPPEMEAENSEVDENVPTAEEATEATEGNAGSAEDTVDISQTGVYTEHVFTDPLGVQIPEDLSPVYQSSNDSDAYKDQISVLPNEQDLVREEAQKMSSLLPTMWLGAQNGCLYVHSSVAQWRKCLHSIKLKDSILSIVHVKGIVLVALADGTLAIFHRGVDGQWDLSNYHLLDLGRPHHSIRCMTVVHDKVWCGYRNKIYVVQPKAMKIEKSFDAHPRKESQVRQLAWVGDGVWVSIRLDSTLRLYHAHTYQHLQDVDIEPYVSKMLGTGKLGFSFVRITALMVSCNRLWVGTGNGVIISIPLTESKYIFR

SEQ ID NO: 200 - DNA, 29 bp, Artificial Sequence
  oligonucleotide
tnaggtttgcacttcttcttgcaaattcc

SEQ ID NO: 201 - DNA, 29 bp, Artificial Sequence
  oligonucleotide
cnaatatctgagtctgtcctgcgggactc

SEQ ID NO: 202 - DNA, 29 bp, Artificial Sequence
  oligonucleotide
cnggctctgtgatggctgaatggaaagaa

SEQ ID NO: 203 - DNA, 29 bp, Artificial Sequence
  oligonucleotide
angagtccaattaaagaacgaaaagcggg

SEQ ID NO: 204 - DNA, 29 bp, Artificial Sequence
  oligonucleotide
cntcagcttgggcaagagtaaagagggca

SEQ ID NO: 205 - DNA, 29 bp, Artificial Sequence
  oligonucleotide
tnatttaagatgctgtgtaagcctctcgc

SEQ ID NO: 206 - DNA, 29 bp, Artificial Sequence
  oligonucleotide
tntctgtgtgcacagagctttggagtggc

SEQ ID NO: 207 - DNA, 29 bp, Artificial Sequence
  oligonucleotide
gnttggattctctggcagagtacacctgc

SEQ ID NO: 208 - DNA, 29 bp, Artificial Sequence
  oligonucleotide
gnaggcatatcttttacaggttcagctcc

SEQ ID NO: 209 - DNA, 29 bp, Artificial Sequence
  oligonucleotide
cncagtgagacaggtagtaggagccagat

SEQ ID NO: 210 - DNA, 29 bp, Artificial Sequence
  oligonucleotide
angtaaattccagccaggatgatggccgc

SEQ ID NO: 211 - DNA, 29 bp, Artificial Sequence
  oligonucleotide
tngagtgtggaattgcgcttcttacctat

SEQ ID NO: 212 - DNA, 29 bp, Artificial Sequence
  oligonucleotide
gngtgaagtcttcctgggaaccataatct

SEQ ID NO: 213 - DNA, 29 bp, Artificial Sequence
  oligonucleotide
tnttccctgaagagctggagaggtgctaa

SEQ ID NO: 214 - DNA, 29 bp, Artificial Sequence
  oligonucleotide
gntctaccatgtgaagaaggaacgcaaaa

SEQ ID NO: 215 - DNA, 29 bp, Artificial Sequence
  oligonucleotide
tnggcaaagctgtgctgcaggatagagtg

SEQ ID NO: 216 - DNA, 29 bp, Artificial Sequence
  oligonucleotide
angtgaaggactgtaagcccacacaagct

SEQ ID NO: 217 - DNA, 29 bp, Artificial Sequence
  oligonucleotide
tnatgacagcccaggctgtgttatctgca

SEQ ID NO: 218 - DNA, 29 bp, Artificial Sequence
  oligonucleotide
cngggccttcgttttcactaccttcagaa

SEQ ID NO: 219 - DNA, 29 bp, Artificial Sequence
  oligonucleotide
tnaagacggtgactgagaggcagacaagg

SEQ ID NO: 220 - DNA, 29 bp, Artificial Sequence
  oligonucleotide
tncctagtccctttaggtagctctgcaga

SEQ ID NO: 221 - DNA, 29 bp, Artificial Sequence
  oligonucleotide
gngcacgagaaagaaggcgaggatggcta

SEQ ID NO: 222 - DNA, 29 bp, Artificial Sequence
  oligonucleotide
tnggacactgtggattctgagtcttgtgg

SEQ ID NO: 223 - DNA, 29 bp, Artificial Sequence
  oligonucleotide
ancaggaggcagtgggttctatagagtcc

SEQ ID NO: 224 - DNA, 29 bp, Artificial Sequence
  oligonucleotide
cncttgtgtgcctgtttctcaattgcaga

SEQ ID NO: 225 - DNA, 29 bp, Artificial Sequence
  oligonucleotide
cnctagctgttcggctttaccaaggagtt

SEQ ID NO: 226 - DNA, 29 bp, Artificial Sequence
  oligonucleotide
ancacgctctgacgaatgagcaacctctc

SEQ ID NO: 227 - DNA, 29 bp, Artificial Sequence
  oligonucleotide
cnaacacagactggaagcctagagtcgct

SEQ ID NO: 228 - DNA, 29 bp, Artificial Sequence
  oligonucleotide
gnaaaccaggaaatgtacactgacacaat

SEQ ID NO: 229 - DNA, 29 bp, Artificial Sequence
  oligonucleotide
tnatcaatactggtgacagtctgctccaa

SEQ ID NO: 230 - DNA, 29 bp, Artificial Sequence
  oligonucleotide
gncataaccctttgcacgaagctgatctt

SEQ ID NO: 231 - DNA, 29 bp, Artificial Sequence
  oligonucleotide
anatttaagcatttaacgtgaaggatagg

SEQ ID NO: 232 - DNA, 29 bp, Artificial Sequence
  oligonucleotide
tnagagactgggaaaccaacagccactgc

SEQ ID NO: 233 - DNA, 29 bp, Artificial Sequence
  oligonucleotide
anggagcaagttcctgagagtatcctgtt

SEQ ID NO: 234 - DNA, 29 bp, Artificial Sequence
  oligonucleotide
anggagcaagttcctgagagtatcctgtt

SEQ ID NO: 235 - DNA, 29 bp, Artificial Sequence
  oligonucleotide
gnatggagggagagagaacacaaattaac

SEQ ID NO: 236 - DNA, 29 bp, Artificial Sequence
  oligonucleotide
gngacagtgttgtgctgaagcaggattcc

SEQ ID NO: 237 - DNA, 29 bp, Artificial Sequence
  oligonucleotide
cntctccaacttccacaatttgctcacta

SEQ ID NO: 238 - DNA, 29 bp, Artificial Sequence
  oligonucleotide
tnggaggaagagtggcagaggagcttata

SEQ ID NO: 239 - DNA, 29 bp, Artificial Sequence
  oligonucleotide
cngtgttttgttcttaggaaggtagaagc

SEQ ID NO: 240 - DNA, 29 bp, Artificial Sequence
  oligonucleotide
cngccagccagcagcaaagcaaattaaga

SEQ ID NO: 241 - DNA, 29 bp, Artificial Sequence
  oligonucleotide
tngaggaaagaagggtggaggtctgcaca

SEQ ID NO: 242 - DNA, 29 bp, Artificial Sequence
  oligonucleotide
cggtgggatttatttaacatgatcttggc

SEQ ID NO: 243 - DNA, 27 bp, Artificial Sequence
  oligonucleotide
ccaatggttatgatgatggttcttcct

SEQ ID NO: 244 - DNA, 29 bp, Artificial Sequence
  oligonucleotide
anggatgccatccagatgaggccacagct

SEQ ID NO: 245 - DNA, 29 bp, Artificial Sequence
  oligonucleotide
ancttcttctgcagctcattcaactcctg

SEQ ID NO: 246 - DNA, 29 bp, Artificial Sequence
  oligonucleotide
gntcctcctagaccacaaagtagaaagca

SEQ ID NO: 247 - DNA, 29 bp, Artificial Sequence
  oligonucleotide
gnctgactttctctccagtttgtgaaatc

SEQ ID NO: 248 - DNA, 29 bp, Artificial Sequence
  oligonucleotide
gngtggggtgaaaagggaatgattaggga

SEQ ID NO: 249 - DNA, 29 bp, Artificial Sequence
  oligonucleotide
tnattcatgggcagctcattggcgggctc

SEQ ID NO: 250 - DNA, 29 bp, Artificial Sequence
  oligonucleotide
cngctgagactcacacactgaatttcctt

SEQ ID NO: 251 - DNA, 29 bp, Artificial Sequence
  oligonucleotide
cntcttggtccggatgatgtttttatcac

SEQ ID NO: 252 - DNA, 29 bp, Artificial Sequence
  oligonucleotide
tnctcggttatatggaggacgaatagact

SEQ ID NO: 253 - DNA, 27 bp, Artificial Sequence
  oligonucleotide
ctctttatgagcatgatatggcttcag

SEQ ID NO: 254 - DNA, 29 bp, Artificial Sequence
  oligonucleotide
cntgctgcccgacttaaactgagaaccaa

SEQ ID NO: 255 - DNA, 29 bp, Artificial Sequence
  oligonucleotide
gnggacaactcccatagttgaggtttgtc

SEQ ID NO: 256 - DNA, 29 bp, Artificial Sequence
  oligonucleotide
tnggaagagtgacccaggggcccaaagca

SEQ ID NO: 257 - DNA, 29 bp, Artificial Sequence
  oligonucleotide
cnctgcaagcgagtggttttcagggctgt

SEQ ID NO: 258 - DNA, 29 bp, Artificial Sequence
  oligonucleotide
tngagccaaggagagagcagacaggctga

SEQ ID NO: 259 - DNA, 29 bp, Artificial Sequence
  oligonucleotide
ancccccagaagtacaaggacatgccaag

SEQ ID NO: 260 - DNA, 29 bp, Artificial Sequence
  oligonucleotide
cnatagcatcagcatcaacatggtgacaa

SEQ ID NO: 261 - DNA, 27 bp, Artificial Sequence
  oligonucleotide
ctccattctccttgactatcttctccc

SEQ ID NO: 262 - DNA, 29 bp, Artificial Sequence
  oligonucleotide
tncctggtgccatcatgattgtatgaggc

SEQ ID NO: 263 - DNA, 29 bp, Artificial Sequence
  oligonucleotide
anaggctcctccattcctacagccatctt

SEQ ID NO: 264 - DNA, 29 bp, Artificial Sequence
  oligonucleotide
cngtccaactgcttgtaggttatagcaga

SEQ ID NO: 265 - DNA, 29 bp, Artificial Sequence
  oligonucleotide
tnctctacttcacccttttcggtgcatcg

SEQ ID NO: 266 - DNA, 29 bp, Artificial Sequence
  oligonucleotide
tncataaaatgaccccacacctcctactg

SEQ ID NO: 267 - DNA, 29 bp, Artificial Sequence
  oligonucleotide
gngaggtagccaggcatctctgtgcattt

SEQ ID NO: 268 - DNA, 29 bp, Artificial Sequence
  oligonucleotide
ancacagcaggaggtgaggaggaggagat

SEQ ID NO: 269 - DNA, 29 bp, Artificial Sequence
  oligonucleotide
cnttgatctgagagcttttcaccgtcaag

SEQ ID NO: 270 - DNA, 29 bp, Artificial Sequence
  oligonucleotide
anctgccaagaagaggagagtggtaaatt

SEQ ID NO: 271 - DNA, 29 bp, Artificial Sequence
  oligonucleotide
angaaaaagaagtgtcatgatccagtaca

SEQ ID NO: 272 - DNA, 29 bp, Artificial Sequence
  oligonucleotide
tnatacttggacaggatagagcgaggttt

SEQ ID NO: 273 - DNA, 29 bp, Artificial Sequence
  oligonucleotide
cncgactgccttaaaatgtaaacctggat

SEQ ID NO: 274 - DNA, 29 bp, Artificial Sequence
  oligonucleotide
tngcactgggaaagtgattgtgaggagta

SEQ ID NO: 275 - DNA, 29 bp, Artificial Sequence
  oligonucleotide
tngtgctgccaaagaacaggagcctcaaa

SEQ ID NO: 276 - DNA, 29 bp, Artificial Sequence
  oligonucleotide
ctgctgctcatttgttcaataatgtcaac

SEQ ID NO: 277 - DNA, 29 bp, Artificial Sequence
  oligonucleotide
tngcactgttgtcatgacggcttggtctc

SEQ ID NO: 278 - DNA, 29 bp, Artificial Sequence
  oligonucleotide
anaacagagcagtccagaggaagaacatc

SEQ ID NO: 279 - DNA, 29 bp, Artificial Sequence
  oligonucleotide
anatgaccagatcctctcagaagatttcc

SEQ ID NO: 280 - DNA, 29 bp, Artificial Sequence
  oligonucleotide
tnccagtgcttgggcatgcatgaaggcta

SEQ ID NO: 281 - DNA, 29 bp, Artificial Sequence
  oligonucleotide
ancagcaaactcttcctttcgcttgctgc

SEQ ID NO: 282 - DNA, 29 bp, Artificial Sequence
  oligonucleotide
tntttctcacaggtcagttcatccacttt

SEQ ID NO: 283 - protein, 57 aa, Homo sapiens
MCRPFLSLLLCSTKCFLLCGLGGTHVTFVSCSKAGATPSSLFSTQHQALSRHPINHC

(19) United States
(12) Patent Application Publication
(10) Pub. No.: US 2006/0063227 A1
(43) Pub. Date: Mar. 23, 2006

(54) Secreted proteins and polynucleotides encoding them

(76) Inventors: Kenneth Jacobs, Newton, MA (US); John M. McCoy, Reading, MA (US); Edward R. LaVallie, Harvard, MA (US); Lisa A. Collins-Racie, Acton, MA (US); Cheryl Evans, Germantown, MD (US); David Merberg, Acton, MA (US); Maurice Treacy, Dun Laoghaire (IE); Michael R. Bowman, Canton, MA (US); Vikki Spaulding, Danville, NH (US); Michael J. Agostino, Andover, MA (US)

Correspondence Address: FITZPATRICK CELLA HARPER & SCINTO, 30 ROCKEFELLER PLAZA, NEW YORK, NY, 10112, US

(21) Appl. No.: 10/745,586

(22) Filed: Dec. 14, 2004

Related U.S. Application Data
(63) Continuation of application No. 09/398,829, filed on Sep. 17, 1999, now abandoned.
Application No. 09/398,829 is a continuation-in-part of application No. 08/943,863, filed on Oct. 3, 1997, now abandoned.
Application No. 09/398,829 is a continuation-in-part of application No. 08/957,182, filed on Jan. 24, 1997, now abandoned.
Application No. 09/398,829 is a continuation-in-part of application No. 08/969,515, filed on Nov. 13, 1997, now abandoned.
Application No. 08/969,515 is a continuation-in-part of application No. 08/749,745, filed on Nov. 15, 1996, now abandoned.
Application No. 08/969,515 is a continuation-in-part of application No. 08/867,678, filed on Jun. 2, 1997, now abandoned.
Application No. 08/867,678 is a continuation-in-part of application No. 08/749,745, filed on Nov. 15, 1996, now abandoned.
Application No. 09/398,829 is a continuation-in-part of application No. 08/971,786, filed on Nov. 17, 1997, now abandoned.
Application No. 08/971,786 is a continuation-in-part of application No. 08/800,826, filed on Feb. 14, 1997, now abandoned.
Application No. 09/398,829 is a continuation-in-part of application No. 09/256,938, filed on Feb. 24, 1999, now abandoned.
Application No. 09/256,938 is a continuation-in-part of application No. 08/984,516, filed on Dec. 3, 1997, now abandoned.
Application No. 09/398,829 is a continuation-in-part of application No. 08/989,232, filed on Dec. 11, 1997, now abandoned.
Application No. 09/398,829 is a continuation-in-part of application No. 08/991,872, filed on Dec. 16, 1997, now abandoned.
Application No. 09/398,829 is a continuation-in-part of application No. 08/783,401, filed on Jan. 13, 1997, now abandoned.
Application No. 09/398,829 is a continuation-in-part of application No. 08/993,228, filed on Dec. 18, 1997, now Pat. No. 5,976,838.
Application No. 08/993,228 is a continuation-in-part of application No. 08/781,255, filed on Jan. 10, 1997, now abandoned.
Application No. 08/781,255 is a continuation-in-part of application No. 08/769,100, filed on Dec. 18, 1996, now abandoned.
Application No. 09/398,829 is a continuation-in-part of application No. 09/360,483, filed on Jul. 23, 1999, now abandoned.
Application No. 09/360,483 is a division of application No. 08/993,228, filed on Dec. 18, 1997, now Pat. No. 5,976,838.
Application No. 08/993,228 is a continuation-in-part of application No. 08/781,225, filed on Jan. 10, 1997, now abandoned.
Application No. 08/781,225 is a continuation-in-part of application No. 08/769,100, filed on Dec. 18, 1996, now abandoned.
(60) Provisional application No. 60/126,450, filed on Oct. 4, 1996.
(60) Provisional application No. 60/086,243, filed on Dec. 6, 1996.
(60) Provisional application No. 60/077,184, filed on Dec. 13, 1996.
(60) Provisional application No. 60/122,050, filed on Dec. 18, 1996.
(60) Provisional application No. 60/122,050, filed on Dec. 18, 1996.

(51) Int. Cl. C12P 21/06

(52) U.S. Cl. 435/69.1

(57) ABSTRACT

Novel polynucleotides and the proteins encoded thereby are disclosed.

This application is a continuation-in-part of the following applications:
(1) Ser. No. 08/943,863 (GI 6011A), filed Oct. 3, 1997; which is a continuation-in-part of provisional application Ser. No. 60/126,450 (GI 6011; converted to a provisional application from non-provisional application Ser. No. 08/726,237), filed Oct. 4, 1996, now abandoned;
(2) Ser. No. 08/957,182 (GI 6012A), filed Oct. 24, 1997; which is a continuation-in-part of provisional application Ser. No. 60/______ (GI 6012; converted to a provisional application from non-provisional application Ser. No. 08/740,274), filed Oct. 25, 1996, now abandoned;
(3) Ser. No. 08/969,515 (GI 6013A), filed Nov. 13, 1997; which is a continuation-in-part of the following applications: (A) Ser. No. 08/749,745 (GI 6013), filed Nov. 15, 1996, now abandoned; and (B) Ser. No. 08/867,678 (GI 6013.BL229), filed Jun. 2, 1997, now abandoned; which was a continuation-in-part of application Ser. No. 08/749,745 (GI 6013), filed Nov. 15, 1996, now abandoned;
(4) Ser. No. 08/971,786 (GI 6014A), filed Nov. 17, 1997; which is a continuation-in-part of the following applications: (A) Ser. No. 60/______ (GI 6014; converted to a provisional application from non-provisional application Ser. No. 08/752,912), filed Nov. 20, 1996, now abandoned; and (B) Ser. No. 08/800,826 (GI 6014.CC182), filed Feb. 14, 1997, now abandoned; which was a continuation-in-part of application Ser. No. 60/______ (GI 6014; converted to a provisional application from non-provisional application Ser. No. 08/752,912), filed Nov. 20, 1996, now abandoned;
(5) Ser. No. 09/256,938 (GI 6015B), filed Feb. 24, 1999; which is a continuation-in-part of application Ser. No. 08/984,516 (GI 6015A), filed Dec. 3, 1997, now abandoned; which was a continuation-in-part of provisional application Ser. No. 60/086,243 (GI 6015; converted to a provisional application from non-provisional application Ser. No. 08/762,216), filed Dec. 6, 1996, now abandoned;
(6) Ser. No. 08/989,232 (GI 6016A), filed Dec. 11, 1997; which is a continuation-in-part of provisional application Ser. No. 60/077,184 (GI 6016; converted to a provisional application from non-provisional application Ser. No. 08/766,263), filed Dec. 13, 1996, now abandoned;
(7) Ser. No. 08/991,872 (GI 6017A), filed Dec. 16, 1997; which is a continuation-in-part of the following applications: (A) Ser. No. 60/122,050 (GI 6017; converted to a provisional application from non-provisional application Ser. No. 08/769,192), filed Dec. 18, 1996, now abandoned; and (B) Ser. No. 08/783,401 (GI 6017.CO223), filed Jan. 13, 1997, now abandoned; which was a continuation-in-part of application Ser. No. 60/122,050 (GI 6017; converted to a provisional application from non-provisional application Ser. No. 08/769,192), filed Dec. 18, 1996, now abandoned;
(8) Ser. No. 08/993,228 (GI 6018B), filed Dec. 18, 1997; which is a continuation-in-part of application Ser. No. 08/781,225 (GI 6018A), filed Jan. 10, 1997, now abandoned; which was a continuation-in-part of application Ser. No. 08/769,100 (GI 6018), filed Dec. 18, 1996, now abandoned; and
(9) Ser. No. 09/360,483 (GI 6018B-DIV), filed Jul. 23, 1999; which is a divisional of application Ser. No. 08/993,228 (GI 6018B), filed Dec. 18, 1997; which is a continuation-in-part of application Ser. No. 08/781,225 (GI 6018A), filed Jan. 10, 1997, now abandoned; which was a continuation-in-part of application Ser. No. 08/769,100 (GI 6018), filed Dec. 18, 1996, now abandoned; all of which are incorporated by reference herein.

### FIELD OF THE INVENTION

The present invention provides novel polynucleotides and proteins encoded by such polynucleotides, along with therapeutic, diagnostic and research utilities for these polynucleotides and proteins.

### BACKGROUND OF THE INVENTION

Technology aimed at the discovery of protein factors (including e.g., cytokines, such as lymphokines, interferons, CSFs and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization cloning techniques, have advanced the state of the art by making available large numbers of DNA/amino acid sequences for proteins that are known to have biological activity by virtue of their secreted nature in the case of leader sequence cloning, or by virtue of the cell or tissue source in the case of PCR-based techniques. It is to these proteins and the polynucleotides encoding them that the present invention is directed.

### SUMMARY OF THE INVENTION

In one embodiment, the present invention provides a composition comprising an isolated polynucleotide selected from the group consisting of:
(a) a polynucleotide comprising the nucleotide sequence of SEQ ID NO:1;
(b) a polynucleotide comprising the nucleotide sequence of SEQ ID NO:1 from nucleotide 282 to nucleotide 565;
(c) a polynucleotide comprising the nucleotide sequence of SEQ ID NO:1 from nucleotide 342 to nucleotide 565;
(d) a polynucleotide comprising the nucleotide sequence of the full-length protein coding sequence of clone AX65_{—}22 deposited with the ATCC under accession number 98196;
(e) a polynucleotide encoding the full-length protein encoded by the cDNA insert of clone AX65_{—}22 deposited with the ATCC under accession number 98196;
(f) a polynucleotide comprising the nucleotide sequence of a mature protein coding sequence of clone AX65_{—}22 deposited with the ATCC under accession number 98196;
(g) a polynucleotide encoding a mature protein encoded by the cDNA insert of clone AX65_{—}22 deposited with the ATCC under accession number 98196;
(h) a polynucleotide encoding a protein comprising the amino acid sequence of SEQ ID NO:2;
(i) a polynucleotide encoding a protein comprising a fragment of the amino acid sequence of SEQ ID NO:2 having biological activity, the fragment comprising eight contiguous amino acids of SEQ ID NO:2;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:1.

Preferably, such polynucleotide comprises the nucleotide sequence of SEQ ID NO:1 from nucleotide 282 to nucleotide 565; the nucleotide sequence of SEQ ID NO:1 from nucleotide 342 to nucleotide 565; the nucleotide sequence of the full-length protein coding sequence of clone AX65_{—}22 deposited with the ATCC under accession number 98196; or the nucleotide sequence of a mature protein coding sequence of clone AX65_{—}22 deposited with the ATCC under accession number 98196. In other preferred embodiments, the polynucleotide encodes the full-length or a mature protein encoded by the cDNA insert of clone AX65_{—}22 deposited with the ATCC under accession number 98196. In further preferred embodiments, the present invention provides a polynucleotide encoding a protein comprising a fragment of the amino acid sequence of SEQ ID NO:2 having biological activity, the fragment preferably comprising eight (more preferably twenty, most preferably thirty) contiguous amino acids of SEQ ID NO:2, or a polynucleotide encoding a protein comprising a fragment of the amino acid sequence of SEQ ID NO:2 having biological activity, the fragment comprising the amino acid sequence from amino acid 42 to amino acid 51 of SEQ ID NO:2.

Other embodiments provide the gene corresponding to the cDNA sequence of SEQ ID NO:1 and SEQ ID NO:3.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:1;
      (ab) SEQ ID NO:3, but excluding the poly(A) tail at the 3′ end of SEQ ID NO:3; and
      (ac) the nucleotide sequence of the cDNA insert of clone AX65_{—}22 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:1;
      (bb) SEQ ID NO:3, but excluding the poly(A) tail at the 3′ end of SEQ ID NO:3; and
      (bc) the nucleotide sequence of the cDNA insert of clone AX65_{—}22 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).

Preferably the polynucleotide isolated according to the above process comprises a nucleotide sequence corresponding to the cDNA sequences of SEQ ID NO:1 and SEQ ID NO:3, and extending contiguously from a nucleotide sequence corresponding to the 5′ end of SEQ ID NO:1 to a nucleotide sequence corresponding to the 3′ end of SEQ ID NO:3, but excluding the poly(A) tail at the 3′ end of SEQ ID NO:3. Also preferably the polynucleotide isolated according to the above process comprises a nucleotide sequence corresponding to the cDNA sequence of SEQ ID NO:1, and extending contiguously from a nucleotide sequence corresponding to the 5′ end of SEQ ID NO:1 to a nucleotide sequence corresponding to the 3′ end of SEQ ID NO:1. Also preferably the polynucleotide isolated according to the above process comprises a nucleotide sequence corresponding to the cDNA sequence of SEQ ID NO:1 from nucleotide 282 to nucleotide 565, and extending contiguously from a nucleotide sequence corresponding to the 5′ end of said sequence of SEQ ID NO:1 from nucleotide 282 to nucleotide 565, to a nucleotide sequence corresponding to the 3′ end of said sequence of SEQ ID NO:1 from nucleotide 282 to nucleotide 565. Also preferably the polynucleotide isolated according to the above process comprises a nucleotide sequence corresponding to the cDNA sequence of SEQ ID NO:1 from nucleotide 342 to nucleotide 565, and extending contiguously from a nucleotide sequence corresponding to the 5′ end of said sequence of SEQ ID NO:1 from nucleotide 342 to nucleotide 565, to a nucleotide sequence corresponding to the 3′ end of said sequence of SEQ ID NO:1 from nucleotide 342 to nucleotide 565.

In other embodiments; the present invention provides a composition comprising a protein, wherein said protein comprises an amino acid sequence selected from the group consisting of:
(a) the amino acid sequence of SEQ ID NO:2;
(b) a fragment of the amino acid sequence of SEQ ID NO:2, the fragment comprising eight contiguous amino acids of SEQ ID NO:2; and
(c) the amino acid sequence encoded by the cDNA insert of clone AX65_{—}22 deposited with the ATCC under accession number 98196;
   the protein being substantially free from other mammalian proteins. Preferably such protein comprises the amino acid sequence of SEQ ID NO:2. In further preferred embodiments, the present invention provides a protein comprising a fragment of the amino acid sequence of SEQ ID NO:2 having biological activity, the fragment preferably comprising eight (more preferably twenty, most preferably thirty) contiguous amino acids of SEQ ID NO:2, or a protein comprising a fragment of the amino acid sequence of SEQ ID NO:2 having biological activity, the fragment comprising the amino acid sequence from amino acid 42 to amino acid 51 of SEQ ID NO:2.

In one embodiment, the present invention provides a composition comprising an isolated polynucleotide selected from the group consisting of:
(a) a polynucleotide comprising the nucleotide sequence of SEQ ID NO:4;
(b) a polynucleotide comprising the nucleotide sequence of SEQ ID NO:4 from nucleotide 192 to nucleotide 2318;
(c) a polynucleotide comprising the nucleotide sequence of SEQ ID NO:4 from nucleotide 653 to nucleotide 825;
(d) a polynucleotide comprising the nucleotide sequence of the full-length protein coding sequence of clone BD335_{—}14 deposited with the ATCC under accession number 98196;
(e) a polynucleotide encoding the full-length protein encoded by the cDNA insert of clone BD335_{—}14 deposited with the ATCC under accession number 98196;
(f) a polynucleotide comprising the nucleotide sequence of a mature protein coding sequence of clone BD335_{—}14 deposited with the ATCC under accession number 98196;
(g) a polynucleotide encoding a mature protein encoded by the cDNA insert of clone BD335_{—}14 deposited with the ATCC under accession number 98196;
(h) a polynucleotide encoding a protein comprising the amino acid sequence of SEQ ID NO:5;
(i) a polynucleotide encoding a protein comprising a fragment of the amino acid sequence of SEQ ID NO:5 having biological activity, the fragment comprising eight contiguous amino acids of SEQ ID NO:5;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a>(i) and that has a length that is at least 25% of the length of SEQ ID NO:4.

Preferably, such polynucleotide comprises the nucleotide sequence of SEQ ID NO:4 from nucleotide 192 to nucleotide 2318; the nucleotide sequence of SEQ ID NO:4 from nucleotide 653 to nucleotide 825; the nucleotide sequence of the full-length protein coding sequence of clone BD335_{—}14 deposited with the ATCC under accession number 98196; or the nucleotide sequence of a mature protein coding sequence of clone BD335_{—}14 deposited with the ATCC under accession number 98196. In other preferred embodiments, the polynucleotide encodes the full-length or a mature protein encoded by the cDNA insert of clone BD33514 deposited with the ATCC under accession number 98196. In yet other preferred embodiments, the present invention provides a polynucleotide encoding a protein comprising the amino acid sequence of SEQ ID NO:5 from amino acid 148 to amino acid 240. In further preferred embodiments, the present invention provides a polynucleotide encoding a protein comprising a fragment of the amino acid sequence of SEQ ID NO:5 having biological activity, the fragment preferably comprising eight (more preferably twenty, most preferably thirty) contiguous amino acids of SEQ ID NO:5, or a polynucleotide encoding a protein comprising a fragment of the amino acid sequence of SEQ ID NO:5 having biological activity, the fragment comprising the amino acid sequence from amino acid 349 to amino acid 358 of SEQ ID NO:5.

Other embodiments provide the gene corresponding to the cDNA sequence of SEQ ID NO:4.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:4, but excluding the poly(A) tail at the 3′ end of SEQ ID NO:4; and
      (ab) the nucleotide sequence of the cDNA insert of clone BD335_{—}14 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:4, but excluding the poly(A) tail at the 3′ end of SEQ ID NO:4; and
      (bb) the nucleotide sequence of the cDNA insert of clone BD335_{—}14 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:4, and extending contiguously from a nucleotide sequence corresponding to the 5′ end of SEQ ID NO:4 to a nucleotide sequence corresponding to the 3′ end of SEQ ID NO:4, but excluding the poly(A) tail at the 3′ end of SEQ ID NO:4. Also preferably the polynucleotide isolated according to the above process comprises a nucleotide sequence corresponding to the cDNA sequence of SEQ ID NO:4 from nucleotide 192 to nucleotide 2318, and extending contiguously from a nucleotide sequence corresponding to the 5′ end of said sequence of SEQ ID NO:4 from nucleotide 192 to nucleotide 2318, to a nucleotide sequence corresponding to the 3′ end of said sequence of SEQ ID NO:4 from nucleotide 192 to nucleotide 2318. Also preferably the polynucleotide isolated according to the above process comprises a nucleotide sequence corresponding to the cDNA sequence of SEQ ID NO:4 from nucleotide 653 to nucleotide 825, and extending contiguously from a nucleotide sequence corresponding to the 5′ end of said sequence of SEQ ID NO:4 from nucleotide 653 to nucleotide 825, to a nucleotide sequence corresponding to the 3′ end of said sequence of SEQ ID NO:4 from nucleotide 653 to nucleotide 825.

In other embodiments, the present invention provides a composition comprising a protein, wherein said protein comprises an amino acid sequence selected from the group consisting of:
(a) the amino acid sequence of SEQ ID NO:5;
(b) the amino acid sequence of SEQ ID NO:5 from amino acid 148 to amino acid 240;
(c) a fragment of the amino acid sequence of SEQ ID NO:5, the fragment comprising eight contiguous amino acids of SEQ ID NO:5; and
(d) the amino acid sequence encoded by the cDNA insert of clone BD335_{—}14 deposited with the ATCC under accession number 98196;
   the protein being substantially free from other mammalian proteins. Preferably such protein comprises the amino acid sequence of SEQ ID NO:5 or the amino acid sequence of SEQ ID NO:5 from amino acid 148 to amino acid 240. In further preferred embodiments, the present invention provides a protein comprising a fragment of the amino acid sequence of SEQ ID NO:5 having biological activity, the fragment preferably comprising eight (more preferably twenty, most preferably thirty) contiguous amino acids of SEQ ID NO:5, or a protein comprising a fragment of the amino acid sequence of SEQ ID NO:5 having biological activity, the fragment comprising the amino acid sequence from amino acid 349 to amino acid 358 of SEQ ID NO:5.

In one embodiment, the present invention provides a composition comprising an isolated polynucleotide selected from the group consisting of:
(a) a polynucleotide comprising the nucleotide sequence of SEQ ID NO:7;
(b) a polynucleotide comprising the nucleotide sequence of SEQ ID NO:7 from nucleotide 206 to nucleotide 391;
(c) a polynucleotide comprising the nucleotide sequence of the full-length protein coding sequence of clone BG241_{—}1 deposited with the ATCC under accession number 98196;
(d) a polynucleotide encoding the full-length protein encoded by the cDNA insert of clone BG241_{—}1 deposited with the ATCC under accession number 98196;
(e) a polynucleotide comprising the nucleotide sequence of a mature protein coding sequence of clone BG241_{—}1 deposited with the ATCC under accession number 98196;
(f) a polynucleotide encoding a mature protein encoded by the cDNA insert of clone BG241_{—}1 deposited with the ATCC under accession number 98196;
(g) a polynucleotide encoding a protein comprising the amino acid sequence of SEQ ID NO:8;
(h) a polynucleotide encoding a protein comprising a fragment of the amino acid sequence of SEQ ID NO:8 having biological activity, the fragment comprising eight contiguous amino acids of SEQ ID NO:8;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:7.

Preferably, such polynucleotide comprises the nucleotide sequence of SEQ ID NO:7 from nucleotide 206 to nucleotide 391; the nucleotide sequence of the full-length protein coding sequence of clone BG241_{—}1 deposited with the ATCC under accession number 98196; or the nucleotide sequence of a mature protein coding sequence of clone BG241_{—}1 deposited with the ATCC under accession number 98196. In other preferred embodiments, the polynucleotide encodes the full-length or a mature protein encoded by the cDNA insert of clone BG241_{—}1 deposited with the ATCC under accession number 98196. In further preferred embodiments, the present invention provides a polynucleotide encoding a protein comprising a fragment of the amino acid sequence of SEQ ID NO:8 having biological activity, the fragment preferably comprising eight (more preferably twenty, most preferably thirty) contiguous amino acids of SEQ ID NO:8, or a polynucleotide encoding a protein comprising a fragment of the amino add sequence of SEQ ID NO:8 having biological activity, the fragment comprising the amino acid sequence from amino acid 26 to amino acid 35 of SEQ ID NO:8.

Other embodiments provide the gene corresponding to the cDNA sequence of SEQ ID NO:7, SEQ ID NO:6, and SEQ ID NO:9.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:6;
      (ab) SEQ ID NO:7;
      (ac) SEQ ID NO:9, but excluding the poly(A) tail at the 3′ end of SEQ ID NO:9; and
      (ad) the nucleotide sequence of the cDNA insert of clone BG241_J deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:6;
      (bb) SEQ ID NO:7;
      (bc) SEQ ID NO:9, but excluding the poly(A) tail at the 3′ end of SEQ ID NO:9; and
      (bd) the nucleotide sequence of the cDNA insert of clone BG241_{—}1 deposited with the ATCC under accession number 98196;
(ii) hybridizing said primer(s) to human genomic DNA in conditions at least as stringent as 4×SSC at 50 degrees C.;
(iii) amplifying human DNA sequences; and
(iv) isolating the polynucleotide products of step (b)(iii).
   Preferably the polynucleotide isolated according to the above process comprises a nucleotide sequence corresponding to the cDNA sequences of SEQ ID NO:6, SEQ ID NO:7, and SEQ ID NO:9, and extending contiguously from a nucleotide sequence corresponding to the 5′ end of SEQ ID NO:6 to a nucleotide sequence corresponding to the 3′ end of SEQ ID NO:9, but excluding the poly(A) tail at the 3′ end of SEQ ID NO:9. Also preferably the polynucleotide isolated according to the above process comprises a nucleotide sequence corresponding to the cDNA sequence of SEQ ID NO:7, and extending contiguously from a nucleotide sequence corresponding to the 5′ end of SEQ ID NO:7 to a nucleotide sequence corresponding to the 3′ end of SEQ ID NO:7. Also preferably the polynucleotide isolated according to the above process comprises a nucleotide sequence corresponding to the cDNA sequence of SEQ ID NO:7 from nucleotide 206 to nucleotide 391, and extending contiguously from a nucleotide sequence corresponding to the 5′ end of said sequence of SEQ ID NO:7 from nucleotide 206 to nucleotide 391, to a nucleotide sequence corresponding to the 3′ end of said sequence of SEQ ID NO:7 from nucleotide 206 to nucleotide 391.

In other embodiments, the present invention provides a composition comprising a protein, wherein said protein comprises an amino acid sequence selected from the group consisting of:
(a) the amino acid sequence of SEQ ID NO:8;
(b) a fragment of the amino acid sequence of SEQ ID NO:8, the fragment comprising eight contiguous amino acids of SEQ ID NO:8; and
(c) the amino acid sequence encoded by the cDNA insert of clone BG241_{—}1 deposited with the ATCC under accession number 98196;
   the protein being substantially free from other mammalian proteins. Preferably such protein comprises the amino acid sequence of SEQ ID NO:8. In further preferred embodiments, the present invention provides a protein comprising a fragment of the amino acid sequence of SEQ ID NO:8 having biological activity, the fragment preferably comprising eight (more preferably twenty, most preferably thirty) contiguous amino acids of SEQ ID NO:8, or a protein comprising a fragment of the amino acid sequence of SEQ ID NO:8 having biological activity, the fragment comprising the amino acid sequence from amino acid 26 to amino acid 35 of SEQ ID NO:8.

In one embodiment, the present invention provides a composition comprising an isolated polynucleotide selected from the group consisting of:
(a) a polynucleotide comprising the nucleotide sequence of SEQ ID NO:10;
(b) a polynucleotide comprising the nucleotide sequence of SEQ ID NO:10 from nucleotide 302 to nucleotide 1762;
(c) a polynucleotide comprising the nucleotide sequence of SEQ ID NO:10 from nucleotide 389 to nucleotide 1762;
(d) a polynucleotide comprising the nucleotide sequence of SEQ ID NO:10 from nucleotide 1723 to nucleotide 2050;
(e) a polynucleotide comprising the nucleotide sequence of the full-length protein coding sequence of clone BL187_{—}4 deposited with the ATCC under accession number 98196;
(f) a polynucleotide encoding the full-length protein encoded by the cDNA insert of clone BL187_{—}4 deposited with the ATCC under accession number 98196;
(g) a polynucleotide comprising the nucleotide sequence of a mature protein coding sequence of clone BL187_{—}4 deposited with the ATCC under accession number 98196;
(h) a polynucleotide encoding a mature protein encoded by the cDNA insert of clone BL187_{—}4 deposited with the ATCC under accession number 98196;
(i) a polynucleotide encoding a protein comprising the amino acid sequence of SEQ ID NO:11;
(j) a polynucleotide encoding a protein comprising a fragment of the amino add sequence of SEQ ID NO:11 having biological activity, the fragment comprising eight contiguous amino acids of SEQ ID NO:11;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:10.

Preferably, such polynucleotide comprises the nucleotide sequence of SEQ ID NO:10 from nucleotide 302 to nucleotide 1762; the nucleotide sequence of SEQ ID NO:10 from nucleotide 389 to nucleotide 1762; the nucleotide sequence of SEQ ID NO:10 from nucleotide 1723 to nucleotide 2050; the nucleotide sequence of the full-length protein coding sequence of clone BL187_{—}4 deposited With the ATCC under accession number 98196; or the nucleotide sequence of a mature protein coding sequence of clone BL187_{—}4 deposited with the ATCC under accession number 98196. In other preferred embodiments, the polynucleotide encodes the full-length or a mature protein encoded by the cDNA insert of clone BL187_{—}4 deposited with the ATCC under accession number 98196. In further preferred embodiments, the present invention provides a polynucleotide encoding a protein comprising a fragment of the amino acid sequence of SEQ ID NO:11 having biological activity, the fragment preferably comprising eight (more preferably twenty, most preferably thirty) contiguous amino acids of SEQ ID NO:11, or a polynucleotide encoding a protein comprising a fragment of the amino acid sequence of SEQ ID NO:11 having biological activity, the fragment comprising the amino acid sequence from amino acid 238 to amino acid 247 of SEQ ID NO:11.

Other embodiments provide the gene corresponding to the cDNA sequence of SEQ ID NO:10.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:10, but excluding the poly(A) tail at the 3′ end of SEQ ID NO:10; and
      (ab) the nucleotide sequence of the cDNA insert of clone BL187_{—}4 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:10, but excluding the poly(A) tail at the 3′ end of SEQ ID NO:10; and
      (bb) the nucleotide sequence of the cDNA insert of clone BL187_{—}4 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:10, and extending contiguously from a nucleotide sequence corresponding to the 5′ end of SEQ ID NO:10 to a nucleotide sequence corresponding to the 3′ end of SEQ ID NO:10, but excluding the poly(A) tail at the 3′ end of SEQ ID NO:10. Also preferably the polynucleotide isolated according to the above process comprises a nucleotide sequence corresponding to the cDNA sequence of SEQ ID NO:10 from nucleotide 302 to nucleotide 1762, and extending contiguously from a nucleotide sequence corresponding to the 5′ end of said sequence of SEQ ID NO:10 from nucleotide 302 to nucleotide 1762, to a nucleotide sequence corresponding to the 3′ end of said sequence of SEQ ID NO:10 from nucleotide 302 to nucleotide 1762. Also preferably the polynucleotide isolated according to the above process comprises a nucleotide sequence corresponding to the cDNA sequence of SEQ ID NO:10 from nucleotide 389 to nucleotide 1762, and extending contiguously from a nucleotide sequence corresponding to the 5′ end of said sequence of SEQ ID NO:10 from nucleotide 389 to nucleotide 1762, to a nucleotide sequence corresponding to the 3′ end of said sequence of SEQ ID NO:10 from nucleotide 389 to nucleotide 1762. Also preferably the polynucleotide isolated according to the above process comprises a nucleotide sequence corresponding to the cDNA sequence of SEQ ID NO:10 from nucleotide 1723 to nucleotide 2050, and extending contiguously from a nucleotide sequence corresponding to the 5′ end of said sequence of SEQ ID NO:10 from nucleotide 1723 to nucleotide 2050, to a nucleotide sequence corresponding to the 3′ end of said sequence of SEQ ID NO:10 from nucleotide 1723 to nucleotide 2050.

In other embodiments, the present invention provides a composition comprising a protein, wherein said protein comprises an amino acid sequence selected from the group consisting of:
(a) the amino acid sequence of SEQ ID NO:11;
(b) a fragment of the amino acid sequence of SEQ ID NO:11, the fragment comprising eight contiguous amino acids of SEQ ID NO:11; and
(c) the amino acid sequence encoded by the cDNA insert of clone BL187_{—}4 deposited with the ATCC under accession number 98196;
   the protein being substantially free from other mammalian proteins. Preferably such protein comprises the amino acid sequence of SEQ ID NO:11. In further preferred embodiments, the present invention provides a protein comprising a fragment of the amino acid sequence of SEQ ID NO:11 having biological activity, the fragment preferably comprising eight (more preferably twenty, most preferably thirty) contiguous amino acids of SEQ ID NO:11, or a protein comprising a fragment of the amino acid sequence of SEQ ID NO:11 having biological activity, the fragment comprising the amino acid sequence from amino acid 238 to amino acid 247 of SEQ ID NO:11.

In one embodiment, the present invention provides a composition comprising an isolated polynucleotide selected from the group consisting of:
(a) a polynucleotide comprising the nucleotide sequence of SEQ ID NO:12;
(b) a polynucleotide comprising the nucleotide sequence of SEQ ID NO:12 from nucleotide 2 to nucleotide 2290;
(c) a polynucleotide comprising the nucleotide sequence of SEQ ID NO:12 from nucleotide 134 to nucleotide 2290;
(d) a polynucleotide comprising the nucleotide sequence of SEQ ID NO:12 from nucleotide 1 to nucleotide 309;
(e) a polynucleotide comprising the nucleotide sequence of the full-length protein coding sequence of clone BL249_{—}18 deposited with the ATCC under accession number 98196;
(f) a polynucleotide encoding the full-length protein encoded by the cDNA insert of clone BL249_{—}18 deposited with the ATCC under accession number 98196;
(g) a polynucleotide comprising the nucleotide sequence of a mature protein coding sequence of clone BL249_{—}18 deposited with the ATCC under accession number 98196;
(h) a polynucleotide encoding a mature protein encoded by the cDNA insert of clone BL249_{—}18 deposited with the ATCC under accession number 98196;
(i) a polynucleotide encoding a protein comprising the amino acid sequence of SEQ ID NO:13;
(j) a polynucleotide encoding a protein comprising a fragment of the amino acid sequence of SEQ ID NO:13 having biological activity, the fragment comprising eight contiguous amino acids of SEQ ID NO:13;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:12.

Preferably, such polynucleotide comprises the nucleotide sequence of SEQ ID NO:12 from nucleotide 2 to nucleotide 2290; the nucleotide sequence of SEQ ID NO:12 from nucleotide 134 to nucleotide 2290; the nucleotide sequence of SEQ ID NO:12 from nucleotide 1 to nucleotide 309; the nucleotide sequence of the full-length protein coding sequence of clone BL249_{—}18 deposited with the ATCC under accession number 98196; or the nucleotide sequence of a mature protein coding sequence of clone BL249_{—}18 deposited with the ATCC under accession number 98196. In other preferred embodiments, the polynucleotide encodes the full-length or a mature protein encoded by the cDNA insert of clone BL249_{—}18 deposited with the ATCC under accession number 98196. In yet other preferred embodiments, the present invention provides a polynucleotide encoding a protein comprising the amino acid sequence of SEQ ID NO:13 from amino acid 3 to amino acid 102. In further preferred embodiments, the present invention provides a polynucleotide encoding a protein comprising a fragment of the amino acid sequence of SEQ ID NO:13 having biological activity, the fragment preferably comprising eight (more preferably twenty, most preferably thirty) contiguous amino acids of SEQ ID NO:13, or a polynucleotide encoding a protein comprising a fragment of the amino acid sequence of SEQ ID NO:13 having biological activity, the fragment comprising the amino acid sequence from amino acid 376 to amino add 385 of SEQ ID NO:13.

Other embodiments provide the gene corresponding to the cDNA sequence of SEQ ID NO:12.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:12, but excluding the poly(A) tail at the 3′ end of SEQ ID NO:12; and
      (ab) the nucleotide sequence of the cDNA insert of clone BL249_{—}18 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:12, but excluding the poly(A) tail at the 3′ end of SEQ ID NO:12; and
      (bb) the nucleotide sequence of the cDNA insert of clone BL249_{—}18 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:12, and extending contiguously from a nucleotide sequence corresponding to the 5′ end of SEQ ID NO:12 to a nucleotide sequence corresponding to the 3′ end of SEQ ID NO:12, but excluding the poly(A) tail at the 3′ end of SEQ ID NO:12. Also preferably the polynucleotide isolated according to the above process comprises a nucleotide sequence corresponding to the cDNA sequence of SEQ ID NO:12 from nucleotide 2 to nucleotide 2290, and extending contiguously from a nucleotide sequence corresponding to the 5′ end of said sequence of SEQ ID NO:12 from nucleotide 2 to nucleotide 2290, to a nucleotide sequence corresponding to the 3′ end of said sequence of SEQ ID NO:12 from nucleotide 2 to nucleotide 2290. Also preferably the polynucleotide isolated according to the above process comprises a nucleotide sequence corresponding to the cDNA sequence of SEQ ID NO:12 from nucleotide 134 to nucleotide 2290, and extending contiguously from a nucleotide sequence corresponding to the 5′ end of said sequence of SEQ ID NO:12 from nucleotide 134 to nucleotide 2290, to a nucleotide sequence corresponding to the 3′ end of said sequence of SEQ ID NO:12 from nucleotide 134 to nucleotide 2290. Also preferably the polynucleotide isolated according to the above process comprises a nucleotide sequence corresponding to the cDNA sequence of SEQ ID NO:12 from nucleotide 1 to nucleotide 309, and extending contiguously from a nucleotide sequence corresponding to the 5′ end of said sequence of SEQ ID NO:12 from nucleotide 1 to nucleotide 309, to a nucleotide sequence corresponding to the 3′ end of said sequence of SEQ ID NO:12 from nucleotide 1 to nucleotide 309.

In other embodiments, the present invention provides a composition comprising a protein, wherein said protein comprises an amino acid sequence selected from the group consisting of:
(a) the amino acid sequence of SEQ ID NO:13;
(b) the amino acid sequence of SEQ ID NO:13 from amino acid 3 to amino acid 102;
(c) a fragment of the amino acid sequence of SEQ ID NO:13, the fragment comprising eight contiguous amino acids of SEQ ID NO:13; and
(d) the amino acid sequence encoded by the cDNA insert of clone BL249_{—}18 deposited with the ATCC under accession number 98196;
   the protein being substantially free from other mammalian proteins. Preferably such protein comprises the amino acid sequence of SEQ ID NO:13 or the amino acid sequence of SEQ ID NO:13 from amino acid 3 to amino acid 102. In further preferred embodiments, the present invention provides a protein comprising a fragment of the amino add sequence of SEQ ID NO:13 having biological activity, the fragment preferably comprising eight (more preferably twenty, most preferably thirty) contiguous amino acids of SEQ ID NO:13, or a protein comprising a fragment of the amino acid sequence of SEQ ID NO:13 having biological activity, the fragment comprising the amino acid sequence from amino acid 376 to amino acid 385 of SEQ ID NO:13.

In one embodiment, the present invention provides a composition comprising an isolated polynucleotide selected from the group consisting of:
(a) a polynucleotide comprising the nucleotide sequence of SEQ ID NO:15;
(b) a polynucleotide comprising the nucleotide sequence of SEQ ID NO:15 from nucleotide 459 to nucleotide 539;
(c) a polynucleotide comprising the nucleotide sequence of the full-length protein coding sequence of clone BO71_{—}1 deposited with the ATCC under accession number 98196;
(d) a polynucleotide encoding the full-length protein encoded by the cDNA insert of clone BO71_{—}1 deposited with the ATCC under accession number 98196;
(e) a polynucleotide comprising the nucleotide sequence of a mature protein coding sequence of clone BO71_{—}1 deposited with the ATCC under accession number 98196;
(f) a polynucleotide encoding a mature protein encoded by the cDNA insert of clone BO71_{—}1 deposited with the ATCC under accession number 98196;
(g) a polynucleotide encoding a protein comprising the amino acid sequence of SEQ ID NO:16;
(h) a polynucleotide encoding a protein comprising a fragment of the amino acid sequence of SEQ ID NO:16 having biological activity, the fragment comprising eight contiguous amino acids of SEQ ID NO:16;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:15.

Preferably, such polynucleotide comprises the nucleotide sequence of SEQ ID NO:15 from nucleotide 459 to nucleotide 539; the nucleotide sequence of the full-length protein coding sequence of clone BO71_{—}1 deposited with the ATCC under accession number 98196; or the nucleotide sequence of a mature protein coding sequence of clone BO71_{—}1 deposited with the ATCC under accession number 98196. In other preferred embodiments, the polynucleotide encodes the full-length or a mature protein encoded by the cDNA insert of clone BO71_{—}1 deposited with the ATCC under accession number 98196. In further preferred embodiments, the present invention provides a polynucleotide encoding a protein comprising a fragment of the amino acid sequence of SEQ ID NO:16 having biological activity, the fragment preferably comprising eight (more preferably twenty, most preferably thirty) contiguous amino acids of SEQ ID NO:16, or a polynucleotide encoding a protein comprising a fragment of the amino acid sequence of SEQ ID NO:16 having biological activity, the fragment comprising the amino acid sequence from amino acid 8 to amino acid 17 of SEQ ID NO:16.

Other embodiments provide the gene corresponding to the cDNA sequence of SEQ ID NO:15, SEQ ID NO:14, and SEQ ID NO:17.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:14;
      (ab) SEQ ID NO:15;
      (ac) SEQ ID NO:17, but excluding the poly(A) tail at the 3′ end of SEQ ID NO:17; and
      (ad) the nucleotide sequence of the cDNA insert of clone BO71_{—}1 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:14;
      (bb) SEQ ID NO:15;
      (bc) SEQ ID NO:17, but excluding the poly(A) tail at the 3′ end of SEQ ID NO:17; and
      (bd) the nucleotide sequence of the cDNA insert of clone BO71_{—}1 deposited with the ATCC under accession number 98196;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).

Preferably the polynucleotide isolated according to the above process comprises a nucleotide sequence corresponding to the cDNA sequences of SEQ ID NO:14, SEQ ID NO:15, and SEQ ID NO:17, and extending contiguously from a nucleotide sequence corresponding to the 5′ end of SEQ ID NO:14 to a nucleotide sequence corresponding to the 3′ end of SEQ ID NO:17, but excluding the poly(A) tail at the 3′ end of SEQ ID NO:17. Also preferably the polynucleotide isolated according to the above process comprises a nucleotide sequence corresponding to the cDNA sequence of SEQ ID NO:15, and extending contiguously from a nucleotide sequence corresponding to the 5′ end of SEQ ID NO:15 to a nucleotide sequence corresponding to the 3′ end of SEQ ID NO:15. Also preferably the polynucleotide isolated according to the above process comprises a nucleotide sequence corresponding to the cDNA sequence of SEQ ID NO:15 from nucleotide 459 to nucleotide 539, and extending contiguously from a nucleotide sequence corresponding to the 5′ end of said sequence of SEQ ID NO:15 from nucleotide 459 to nucleotide 539, to a nucleotide sequence corresponding to the 3′ end of said sequence of SEQ ID NO:15 from nucleotide 459 to nucleotide 539.

In other embodiments, the present invention provides a composition comprising a protein, wherein said protein comprises an amino acid sequence selected from the group consisting of:
(a) the amino acid sequence of SEQ ID NO:16;
(b) a fragment of the amino add sequence of SEQ ID NO:16, the fragment comprising eight contiguous amino acids of SEQ ID NO:16; and
(c) the amino acid sequence encoded by the cDNA insert of clone BO71_{—}1 deposited with the ATCC under accession number 98196;
   the protein being substantially free from other mammalian proteins. Preferably such protein comprises the amino acid sequence of SEQ ID NO:16. In further preferred embodiments, the present invention provides a protein comprising a fragment of the amino acid sequence of SEQ ID NO:16 having biological activity, the fragment preferably comprising eight (more preferably twenty, most preferably thirty) contiguous amino acids of SEQ ID NO:16, or a protein comprising a fragment of the amino acid sequence of SEQ ID NO:16 having biological activity, the fragment comprising the amino acid sequence from amino acid 8 to amino acid 17 of SEQ ID NO:16.

In one embodiment, the present invention provides a composition comprising an isolated polynucleotide selected from the group consisting of:
(a) a polynucleotide comprising the nucleotide sequence of SEQ ID NO:18;
(b) a polynucleotide comprising the nucleotide sequence of SEQ ID NO:18 from nucleotide 1237 to nucleotide 1944;
(c) a polynucleotide comprising the nucleotide sequence of SEQ ID NO:18 from nucleotide 737 to nucleotide 1072;
(d) a polynucleotide comprising the nucleotide sequence of the full-length protein coding sequence of clone BO365_{—}2 deposited with the ATCC under accession number 98196;
(e) a polynucleotide encoding the full-length protein encoded by the cDNA insert of clone BO365_{—}2 deposited with the ATCC under accession number 98196;
(f) a polynucleotide comprising the nucleotide sequence of a mature protein coding sequence of clone BO365_{—}2 deposited with the ATCC under accession number 98196;
(g) a polynucleotide encoding a mature protein encoded by the cDNA insert of clone BO365_{—}2 deposited with the ATCC under accession number 98196;
(h) a polynucleotide encoding a protein comprising the amino acid sequence of SEQ ID NO:19;
(i) a polynucleotide encoding a protein comprising a fragment of the amino acid sequence of SEQ ID NO:19 having biological activity, the fragment comprising eight contiguous amino acids of SEQ ID NO:19;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:18.

Preferably, such polynucleotide comprises the nucleotide sequence of SEQ ID NO:18 from nucleotide 1237 to nucleotide 1944; the nucleotide sequence of SEQ ID NO:18 from nucleotide 737 to nucleotide 1072; the nucleotide sequence of the full-length protein coding sequence of clone BO365_{—}2 deposited with the ATCC under accession number 98196; or the nucleotide sequence of a mature protein coding sequence of clone BO365_{—}2 deposited with the ATCC under accession number 98196. In other preferred embodiments, the polynucleotide encodes the full-length or a mature protein encoded by the cDNA insert of clone BO365_{—}2 deposited with the ATCC under accession number 98196. In further preferred embodiments, the present invention provides a polynucleotide encoding a protein comprising a fragment of the amino acid sequence of SEQ ID NO:19 having biological activity, the fragment preferably comprising eight (more preferably twenty, most preferably thirty) contiguous amino acids of SEQ ID NO:19, or a polynucleotide encoding a protein comprising a fragment of the amino acid sequence of SEQ ID NO:19 having biological activity, the fragment comprising the amino acid sequence from amino acid 113 to amino acid 122 of SEQ ID NO:19.

Other embodiments provide the gene corresponding to the cDNA sequence of SEQ ID NO:18.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:18, but excluding the poly(A) tail at the 3′ end of SEQ ID NO:18; and
      (ab) the nucleotide sequence of the cDNA insert of clone BO365_{—}2 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:18, but excluding the poly(A) tail at the 3′ end of SEQ ID NO:18; and
      (bb) the nucleotide sequence of the cDNA insert of clone BO365_{—}2 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:18, and extending contiguously from a nucleotide sequence corresponding to the 5′ end of SEQ ID NO:18 to a nucleotide sequence corresponding to the 3′ end of SEQ ID NO:18, but excluding the poly(A) tail at the 3′ end of SEQ ID NO:18. Also preferably the polynucleotide isolated according to the above process comprises a nucleotide sequence corresponding to the cDNA sequence of SEQ ID NO:18 from nucleotide 1237 to nucleotide 1944, and extending contiguously from a nucleotide sequence corresponding to the 5′ end of said sequence of SEQ ID NO:18 from nucleotide 1237 to nucleotide 1944, to a nucleotide sequence corresponding to the 3′ end of said sequence of SEQ ID NO:18 from nucleotide 1237 to nucleotide 1944. Also preferably the polynucleotide isolated according to the above process comprises a nucleotide sequence corresponding to the cDNA sequence of SEQ ID NO:18 from nucleotide 737 to nucleotide 1072, and extending contiguously from a nucleotide sequence corresponding to the 5′ end of said sequence of SEQ ID NO:18 from nucleotide 737 to nucleotide 1072, to a nucleotide sequence corresponding to the 3′ end of said sequence of SEQ ID NO:18 from nucleotide 737 to nucleotide 1072.

In other embodiments, the present invention provides a composition comprising a protein, wherein said protein comprises an amino acid sequence selected from the group consisting of:
(a) the amino acid sequence of SEQ ID NO:19;
(b) a fragment of the amino acid sequence of SEQ ID NO:19, the fragment comprising eight contiguous amino acids of SEQ ID NO:19; and
(c) the amino acid sequence encoded by the cDNA insert of clone BO365_{—}2 deposited with the ATCC under accession number 98196;
   the protein being substantially free from other mammalian proteins. Preferably such protein comprises the amino acid sequence of SEQ ID NO:19. In further preferred embodiments, the present invention provides a protein comprising a fragment of the amino acid sequence of SEQ ID NO:19 having biological activity, the fragment preferably comprising eight (more preferably twenty, most preferably thirty) contiguous amino adds of SEQ ID NO:19, or a protein comprising a fragment of the amino acid sequence of SEQ ID NO:19 having biological activity, the fragment comprising the amino add sequence from amino acid 113 to amino add 122 of SEQ ID NO:19.

In one embodiment, the present invention provides a composition comprising an isolated polynucleotide selected from the group consisting of:
(a) a polynucleotide comprising the nucleotide sequence of SEQ ID NO:20;
(b) a polynucleotide comprising the nucleotide sequence of SEQ ID NO:20 from nucleotide 68 to nucleotide 328;
(c) a polynucleotide comprising the nucleotide sequence of the full-length protein coding sequence of clone BV51_{—}1 deposited with the ATCC under accession number 98196;
(d) a polynucleotide encoding the full-length protein encoded by the cDNA insert of clone BV51_{—}1 deposited with the ATCC under accession number 98196;
(e) a polynucleotide comprising the nucleotide sequence of a mature protein coding sequence of clone BV51_{—}1 deposited with the ATCC under accession number 98196;
(f) a polynucleotide encoding a mature protein encoded by the cDNA insert of clone BV51_{—}1 deposited with the ATCC under accession number 98196;
(g) a polynucleotide encoding a protein comprising the amino acid sequence of SEQ ID NO:21;
(h) a polynucleotide encoding a protein comprising a fragment of the amino acid sequence of SEQ ID NO:21 having biological activity, the fragment comprising eight contiguous amino acids of SEQ ID NO:21;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:20.

Preferably, such polynucleotide comprises the nucleotide sequence of SEQ ID NO:20 from nucleotide 68 to nucleotide 328; the nucleotide sequence of the full-length protein coding sequence of clone BV51_{—}1 deposited with the ATCC under accession number 98196; or the nucleotide sequence of a mature protein coding sequence of clone BV51_{—}1 deposited with the ATCC under accession number 98196. In other preferred embodiments, the polynucleotide encodes the full-length or a mature protein encoded by the cDNA insert of clone BV51_{—}1 deposited with the ATCC under accession number 98196. In further preferred embodiments, the present invention provides a polynucleotide encoding a protein comprising a fragment of the amino acid sequence of SEQ ID NO:21 having biological activity, the fragment preferably comprising eight (more preferably twenty, most preferably thirty) contiguous amino acids of SEQ ID NO:21, or a polynucleotide encoding a protein comprising a fragment of the amino acid sequence of SEQ ID NO:21 having biological activity, the fragment comprising the amino acid sequence from amino acid 38 to amino acid 47 of SEQ ID NO:21.

Other embodiments provide the gene corresponding to the cDNA sequence of SEQ ID NO:20 and SEQ ID NO:22.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:20;
      (ab) SEQ ID NO:22, but excluding the poly(A) tail at the 3′ end of SEQ ID NO:22; and
      (ac) the nucleotide sequence of the cDNA insert of clone BV51_{—}1 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:20;
      (bb) SEQ ID NO:22, but excluding the poly(A) tail at the 3′ end of SEQ ID NO:22; and
      (bc) the nucleotide sequence of the cDNA insert of clone BV51_{—}1 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:20 and SEQ ID NO:22, and extending contiguously from a nucleotide sequence corresponding to the 5′ end of SEQ ID NO:20 to a nucleotide sequence corresponding to the 3′ end of SEQ ID NO:22, but excluding the poly(A) tail at the 3′ end of SEQ ID NO:22. Also preferably the polynucleotide isolated according to the above process comprises a nucleotide sequence corresponding to the cDNA sequence of SEQ ID NO:20, and extending contiguously from a nucleotide sequence corresponding to the 5′ end of SEQ ID NO:20 to a nucleotide sequence corresponding to the 3′ end of SEQ ID NO:20. Also preferably the polynucleotide isolated according to the above process comprises a nucleotide sequence corresponding to the cDNA sequence of SEQ ID NO:20 from nucleotide 68 to nucleotide 328, and extending contiguously from a nucleotide sequence corresponding to the 5′ end of said sequence of SEQ ID NO:20 from nucleotide 68 to nucleotide 328, to a nucleotide sequence corresponding to the 3′ end of said sequence of SEQ ID NO:20 from nucleotide 68 to nucleotide 328.

In other embodiments, the present invention provides a composition comprising a protein, wherein said protein comprises an amino acid sequence selected from the group consisting of:
(a) the amino acid sequence of SEQ ID NO:21;
(b) a fragment of the amino acid sequence of SEQ ID NO:21, the fragment comprising eight contiguous amino acids of SEQ ID NO:21; and
(c) the amino acid sequence encoded by the cDNA insert of clone BV51_{—}1 deposited with the ATCC under accession number 98196;
   the protein being substantially free from other mammalian proteins. Preferably such protein comprises the amino acid sequence of SEQ ID NO:21. In further preferred embodiments, the present invention provides a protein comprising a fragment of the amino acid sequence of SEQ ID NO:21 having biological activity, the fragment preferably comprising eight (more preferably twenty, most preferably thirty) contiguous amino acids of SEQ ID NO:21, or a protein comprising a fragment of the amino acid sequence of SEQ ID NO:21 having biological activity, the fragment comprising the amino acid sequence from amino acid 38 to amino acid 47 of SEQ ID NO:21.

In one embodiment, the present invention provides a composition comprising an isolated polynucleotide selected from the group consisting of:
(a) a polynucleotide comprising the nucleotide sequence of SEQ ID NO:24;
(b) a polynucleotide comprising the nucleotide sequence of SEQ ID NO:24 from nucleotide 57 to nucleotide 396;
(c) a polynucleotide comprising the nucleotide sequence of the full-length protein coding sequence of clone BV140_{—}3 deposited with the ATCC under accession number 98196;
(d) a polynucleotide encoding the full-length protein encoded by the cDNA insert of clone BV140_{—}3 deposited with the ATCC under accession number 98196;
(e) a polynucleotide comprising the nucleotide sequence of a mature protein coding sequence of clone BV140_{—}3 deposited with the ATCC under accession number 98196;
(f) a polynucleotide encoding a mature protein encoded by the cDNA insert of clone BV140_{—}3 deposited with the ATCC under accession number 98196;
(g) a polynucleotide encoding a protein comprising the amino add sequence of SEQ ID NO:25;
(h) a polynucleotide encoding a protein comprising a fragment of the amino acid sequence of SEQ ID NO:25 having biological activity, the fragment comprising eight contiguous amino acids of SEQ ID NO:25;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:24.

Preferably, such polynucleotide comprises the nucleotide sequence of SEQ ID NO:24 from nucleotide 57 to nucleotide 396; the nucleotide sequence of the full-length protein coding sequence of clone BV140_{—}3 deposited with the ATCC under accession number 98196; or the nucleotide sequence of a mature protein coding sequence of clone BV140_{—}3 deposited with the ATCC under accession number 98196. In other preferred embodiments, the polynucleotide encodes the full-length or a mature protein encoded by the cDNA insert of clone BV140_{—}3 deposited with the ATCC under accession number 98196. In yet other preferred embodiments, the present invention provides a polynucleotide encoding a protein comprising the amino acid sequence of SEQ ID NO:25 from amino acid 29 to amino add 57. In further preferred embodiments, the present invention provides a polynucleotide encoding a protein comprising a fragment of the amino acid sequence of SEQ ID NO:25 having biological activity, the fragment preferably comprising eight (more preferably twenty, most preferably thirty) contiguous amino acids of SEQ ID NO:25, or a polynucleotide encoding a protein comprising a fragment of the amino acid sequence of SEQ ID NO:25 having biological activity, the fragment comprising the amino acid sequence from amino acid 51 to amino acid 60 of SEQ ID NO:25.

Other embodiments provide the gene corresponding to the cDNA sequence of SEQ ID NO:24, SEQ ID NO:23, and SEQ ID NO:26.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:23;
      (ab) SEQ ID NO:24;
      (ac) SEQ ID NO:26, but excluding the poly(A) tail at the 3′ end of SEQ ID NO:26; and
      (ad) the nucleotide sequence of the cDNA insert of clone BV140_{—}3 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:23;
      (bb) SEQ ID NO:24;
      (bc) SEQ ID NO:26, but excluding the poly(A) tail at the 3′ end of SEQ ID NO:26; and
      (bd) the nucleotide sequence of the cDNA insert of clone BV140_{—}3 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:23, SEQ ID NO:24, and SEQ ID NO:26, and extending contiguously from a nucleotide sequence corresponding to the 5′ end of SEQ ID NO:23 to a nucleotide sequence corresponding to the 3′ end of SEQ ID NO:26, but excluding the poly(A) tail at the 3′ end of SEQ ID NO:26. Also preferably the polynucleotide isolated according to the above process comprises a nucleotide sequence corresponding to the cDNA sequence of SEQ ID NO:24, and extending contiguously from a nucleotide sequence corresponding to the 5′ end of SEQ ID NO:24 to a nucleotide sequence corresponding to the 3′ end of SEQ ID NO:24. Also preferably the polynucleotide isolated according to the above process comprises a nucleotide sequence corresponding to the cDNA sequence of SEQ ID NO:24 from nucleotide 57 to nucleotide 396, and extending contiguously from a nucleotide sequence corresponding to the 5′ end of said sequence of SEQ ID NO:24 from nucleotide 57 to nucleotide 396, to a nucleotide sequence corresponding to the 3′ end of said sequence of SEQ ID NO:24 from nucleotide 57 to nucleotide 396.

In other embodiments, the present invention provides a composition comprising a protein, wherein said protein comprises an amino acid sequence selected from the group consisting of:
(a) the amino acid sequence of SEQ ID NO:25;
(b) the amino acid sequence of SEQ ID NO:25 from amino acid 29 to amino acid 57;
(c) a fragment of the amino acid sequence of SEQ ID NO:25, the fragment comprising eight contiguous amino acids of SEQ ID NO:25; and
(d) the amino acid sequence encoded by the cDNA insert of clone BV140_{—}3 deposited with the ATCC under accession number 98196;
   the protein being substantially free from other mammalian proteins. Preferably such protein comprises the amino add sequence of SEQ ID NO:25 or the amino add sequence of SEQ ID NO:25 from amino acid 29 to amino acid 57. In further preferred embodiments, the present invention provides a protein comprising a fragment of the amino acid sequence of SEQ ID NO:25 having biological activity, the fragment preferably comprising eight (more preferably twenty, most preferably thirty) contiguous amino acids of SEQ ID NO:25, or a protein comprising a fragment of the amino acid sequence of SEQ ID NO:25 having biological activity, the fragment comprising the amino acid sequence from amino acid 51 to amino acid 60 of SEQ ID NO:25.

In one embodiment, the present invention provides a composition comprising an isolated polynucleotide selected from the group consisting of:
(a) a polynucleotide comprising the nucleotide sequence of SEQ ID NO:27;
(b) a polynucleotide comprising the nucleotide sequence of SEQ ID NO:27 from nucleotide 101 to nucleotide 328;
(c) a polynucleotide comprising the nucleotide sequence of SEQ ID NO:27 from nucleotide 1 to nucleotide 197;
(d) a polynucleotide comprising the nucleotide sequence of the full-length protein coding sequence of clone BV141_{—}2 deposited with the ATCC under accession number 98196;
(e) a polynucleotide encoding the full-length protein encoded by the cDNA insert of clone BV141_{—}2 deposited with the ATCC under accession number 98196;
(f) a polynucleotide comprising the nucleotide sequence of a mature protein coding sequence of clone BV141_{—}2 deposited with the ATCC under accession number 98196;
(g) a polynucleotide encoding a mature protein encoded by the cDNA insert of clone BV141_{—}2 deposited with the ATCC under accession number 98196;
(h) a polynucleotide encoding a protein comprising the amino acid sequence of SEQ ID NO:28;
(i) a polynucleotide encoding a protein comprising a fragment of the amino acid sequence of SEQ ID NO:28 having biological activity, the fragment comprising eight contiguous amino acids of SEQ ID NO:28;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:27.

Preferably, such polynucleotide comprises the nucleotide sequence of SEQ ID NO:27 from nucleotide 101 to nucleotide 328; the nucleotide sequence of SEQ ID NO:27 from nucleotide 1 to nucleotide 197; the nucleotide sequence of the full-length protein coding sequence of clone BV141_{—}2 deposited with the ATCC under accession number 98196; or the nucleotide sequence of a mature protein coding sequence of clone BV141_{—}2 deposited with the ATCC under accession number 98196. In other preferred embodiments, the polynucleotide encodes the full-length or a mature protein encoded by the cDNA insert of clone BV141_{—}2 deposited with the ATCC under accession number 98196. In yet other preferred embodiments, the present invention provides a polynucleotide encoding a protein comprising the amino acid sequence of SEQ ID NO:28 from amino acid 1 to amino acid 37. In further preferred embodiments, the present invention provides a polynucleotide encoding a protein comprising a fragment of the amino acid sequence of SEQ ID NO:28 having biological activity, the fragment preferably comprising eight (more preferably twenty, most preferably thirty) contiguous amino acids of SEQ ID NO:28, or a polynucleotide encoding a protein comprising a fragment of the amino add sequence of SEQ ID NO:28 having biological activity, the fragment comprising the amino acid sequence from amino acid 33 to amino acid 42 of SEQ ID NO:28.

Other embodiments provide the gene corresponding to the cDNA sequence of SEQ ID NO:27.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:27, but excluding the poly(A) tail at the 3′ end of SEQ ID NO:27; and
      (ab) the nucleotide sequence of the cDNA insert of clone BV141_{—}2 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:27, but excluding the poly(A) tail at the 3′ end of SEQ ID NO:27; and
      (bb) the nucleotide sequence of the cDNA insert of clone BV141_{—}2 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:27, and extending contiguously from a nucleotide sequence corresponding to the 5′ end of SEQ ID NO:27 to a nucleotide sequence corresponding to the 3′ end of SEQ ID NO:27, but excluding the poly(A) tail at the 3′ end of SEQ ID NO:27. Also preferably the polynucleotide isolated according to the above process comprises a nucleotide sequence corresponding to the cDNA sequence of SEQ ID NO:27 from nucleotide 101 to nucleotide 328, and extending contiguously from a nucleotide sequence corresponding to the 5′ end of said sequence of SEQ ID NO:27 from nucleotide 101 to nucleotide 328, to a nucleotide sequence corresponding to the 3′ end of said sequence of SEQ ID NO:27 from nucleotide 101 to nucleotide 328. Also preferably the polynucleotide isolated according to the above process comprises a nucleotide sequence corresponding to the cDNA sequence of SEQ ID NO:27 from nucleotide 1 to nucleotide 197, and extending contiguously from a nucleotide sequence corresponding to the 5′ end of said sequence of SEQ ID NO:27 from nucleotide 1 to nucleotide 197, to a nucleotide sequence corresponding to the 3′ end of said sequence of SEQ ID NO:27 from nucleotide 1 to nucleotide 197.

In other embodiments, the present invention provides a composition comprising a protein, wherein said protein comprises an amino add sequence selected from the group consisting of:
(a) the amino acid sequence of SEQ ID NO:28;
(b) the amino acid sequence of SEQ ID NO:28 from amino acid 1 to amino acid 37;
(c) a fragment of the amino acid sequence of SEQ ID NO:28, the fragment comprising eight contiguous amino acids of SEQ ID NO:28; and
(d) the amino acid sequence encoded by the cDNA insert of clone BV141_{—}2 deposited with the ATCC under accession number 98196;
   the protein being substantially free from other mammalian proteins. Preferably such protein comprises the amino acid sequence of SEQ ID NO:28 or the amino acid sequence of SEQ ID NO:28 from amino acid 1 to amino acid 37. In further preferred embodiments, the present invention provides a protein comprising a fragment of the amino acid sequence of SEQ ID NO:28 having biological activity, the fragment preferably comprising eight (more preferably twenty, most preferably thirty) contiguous amino acids of SEQ ID NO:28, or a protein comprising a fragment of the amino acid sequence of SEQ ID NO:28 having biological activity, the fragment comprising the amino acid sequence from amino acid 33 to amino acid 42 of SEQ ID NO:28.

In one embodiment, the present invention provides a composition comprising an isolated polynucleotide selected from the group consisting of:
(a) a polynucleotide comprising the nucleotide sequence of SEQ ID NO:29;
(b) a polynucleotide comprising the nucleotide sequence of SEQ ID NO:29 from nucleotide 28 to nucleotide 351;
(c) a polynucleotide comprising the nucleotide sequence of SEQ ID NO:29 from nucleotide 328 to nucleotide 351;
(d) a polynucleotide comprising the nucleotide sequence of the full-length protein coding sequence of clone CC194_{—}4 deposited with the ATCC under accession number 98196;
(e) a polynucleotide encoding the full-length protein encoded by the cDNA insert of clone CC194_{—}4 deposited with the ATCC under accession number 98196;
(f) a polynucleotide comprising the nucleotide sequence of a mature protein coding sequence of clone CC194_{—}4 deposited with the ATCC under accession number 98196;
(g) a polynucleotide encoding a mature protein encoded by the cDNA insert of clone CC194_{—}4 deposited with the ATCC under accession number 98196;
(h) a polynucleotide encoding a protein comprising the amino acid sequence of SEQ ID NO:30;
(i) a polynucleotide encoding a protein comprising a fragment of the amino acid sequence of SEQ ID NO:30 having biological activity, the fragment comprising eight contiguous amino acids of SEQ ID NO:30;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:29.

Preferably, such polynucleotide comprises the nucleotide sequence of SEQ ID NO:29 from nucleotide 28 to nucleotide 351; the nucleotide sequence of SEQ ID NO:29 from nucleotide 328 to nucleotide 351; the nucleotide sequence of the full-length protein coding sequence of clone CC194_{—}4 deposited with the ATCC under accession number 98196; or the nucleotide sequence of a mature protein coding sequence of clone CC194_{—}4 deposited with the ATCC under accession number 98196. In other preferred embodiments, the polynucleotide encodes the full-length or a mature protein encoded by the cDNA insert of clone CC194_{—}4 deposited with the ATCC under accession number 98196. In yet other preferred embodiments, the present invention provides a polynucleotide encoding a protein comprising the amino acid sequence of SEQ ID NO:30 from amino acid 56 to amino add 108. In further preferred embodiments, the present invention provides a polynucleotide encoding a protein comprising a fragment of the amino add sequence of SEQ ID NO:30 having biological activity, the fragment preferably comprising eight (more preferably twenty, most preferably thirty) contiguous amino acids of SEQ ID NO:30, or a polynucleotide encoding a protein comprising a fragment of the amino acid sequence of SEQ ID NO:30 having biological activity, the fragment comprising the amino acid sequence from amino acid 49 to amino acid 58 of SEQ ID NO:30.

Other embodiments provide the gene corresponding to the cDNA sequence of SEQ ID NO:29 and SEQ ID NO:31.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:29;
      (ab) SEQ ID NO:31, but excluding the poly(A) tail at the 3′ end of SEQ ID NO:31; and
      (ac) the nucleotide sequence of the cDNA insert of clone CC194_{—}4 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:29;
      (bb) SEQ ID NO:31, but excluding the poly(A) tail at the 3′ end of SEQ ID NO:31; and
      (bc) the nucleotide sequence of the cDNA insert of clone CC194_{—}4 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:29 and SEQ ID NO:31, and extending contiguously from a nucleotide sequence corresponding to the 5′ end of SEQ ID NO:29 to a nucleotide sequence corresponding to the 3′ end of SEQ ID NO:31, but excluding the poly(A) tail at the 3′ end of SEQ ID NO:31. Also preferably the polynucleotide isolated according to the above process comprises a nucleotide sequence corresponding to the cDNA sequence of SEQ ID NO:29, and extending contiguously from a nucleotide sequence corresponding to the 5′ end of SEQ ID NO:29 to a nucleotide sequence corresponding to the 3′ end of SEQ ID NO:29. Also preferably the polynucleotide isolated according to the above process comprises a nucleotide sequence corresponding to the cDNA sequence of SEQ ID NO:29 from nucleotide 28 to nucleotide 351, and extending contiguously from a nucleotide sequence corresponding to the 5′ end of said sequence of SEQ ID NO:29 from nucleotide 28 to nucleotide 351, to a nucleotide sequence corresponding to the 3′ end of said sequence of SEQ ID NO:29 from nucleotide 28 to nucleotide 351. Also preferably the polynucleotide isolated according to the above process comprises a nucleotide sequence corresponding to the cDNA sequence of SEQ ID NO:29 from nucleotide 328 to nucleotide 351, and extending contiguously from a nucleotide sequence corresponding to the 5′ end of said sequence of SEQ ID NO:29 from nucleotide 328 to nucleotide 351, to a nucleotide sequence corresponding to the 3′ end of said sequence of SEQ ID NO:29 from nucleotide 328 to nucleotide 351.

In other embodiments, the present invention provides a composition comprising a protein, wherein said protein comprises an amino acid sequence selected from the group consisting of:
(a) the amino acid sequence of SEQ ID NO:30;
(b) the amino acid sequence of SEQ ID NO:30 from amino acid 56 to amino acid 108;
(c) a fragment of the amino acid sequence of SEQ ID NO:30, the fragment comprising eight contiguous amino acids of SEQ ID NO:30; and
(d) the amino acid sequence encoded by the cDNA insert of clone CC194_{—}4 deposited with the ATCC under accession number 98196;
   the protein being substantially free from other mammalian proteins. Preferably such protein comprises the amino acid sequence of SEQ ID NO:30 or the amino acid sequence of SEQ ID NO:30 from amino acid 56 to amino acid 108. In further preferred embodiments, the present invention provides a protein comprising a fragment of the amino acid sequence of SEQ ID NO:30 having biological activity, the fragment preferably comprising eight (more preferably twenty, most preferably thirty) contiguous amino acids of SEQ ID NO:30, or a protein comprising a fragment of the amino acid sequence of SEQ ID NO:30 having biological activity, the fragment comprising the amino acid sequence from amino acid 49 to amino acid 58 of SEQ ID NO:30.

In one embodiment, the present invention provides a composition comprising an isolated polynucleotide selected from the group consisting of:
(a) a polynucleotide comprising the nucleotide sequence of SEQ ID NO:32;
(b) a polynucleotide comprising the nucleotide sequence of SEQ ID NO:32 from nucleotide 338 to nucleotide 1198;
(c) a polynucleotide comprising the nucleotide sequence of SEQ ID NO:32 from nucleotide 467 to nucleotide 1058;
(d) a polynucleotide comprising the nucleotide sequence of the full-length protein coding sequence of clone DA136_{—}11 deposited with the ATCC under accession number 98196;
(e) a polynucleotide encoding the full-length protein encoded by the cDNA insert of clone DA136_{—}11 deposited with the ATCC under accession number 98196;
(f) a polynucleotide comprising the nucleotide sequence of a mature protein coding sequence of clone DA136_{—}11 deposited with the ATCC under accession number 98196;
(g) a polynucleotide encoding a mature protein encoded by the cDNA insert of clone DA136_{—}11 deposited with the ATCC under accession number 98196;
(h) a polynucleotide encoding a protein comprising the amino acid sequence of SEQ ID NO:33;
(i) a polynucleotide encoding a protein comprising a fragment of the amino acid sequence of SEQ ID NO:33 having biological activity, the fragment comprising eight contiguous amino acids of SEQ ID NO:33;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:32.

Preferably, such polynucleotide comprises the nucleotide sequence of SEQ ID NO:32 from nucleotide 338 to nucleotide 1198; the nucleotide sequence of SEQ ID NO:32 from nucleotide 467 to nucleotide 1058; the nucleotide sequence of the full-length protein coding sequence of clone DA136_{—}11 deposited with the ATCC under accession number 98196; or the nucleotide sequence of a mature protein coding sequence of clone DA136_{—}11 deposited with the ATCC under accession number 98196. In other preferred embodiments, the polynucleotide encodes the full-length or a mature protein encoded by the cDNA insert of clone DA136_{—}11 deposited with the ATCC under accession number 98196. In yet other preferred embodiments, the present invention provides a polynucleotide encoding a protein comprising the amino acid sequence of SEQ ID NO:33 from amino acid 124 to amino acid 182. In further preferred embodiments, the present invention provides a polynucleotide encoding a protein comprising a fragment of the amino acid sequence of SEQ ID NO:33 having biological activity, the fragment preferably comprising eight (more preferably twenty, most preferably thirty) contiguous amino acids of SEQ ID NO:33, or a polynucleotide encoding a protein comprising a fragment of the amino acid sequence of SEQ ID NO:33 having biological activity, the fragment comprising the amino acid sequence from amino acid 138 to amino acid 147 of SEQ ID NO:33.

Other embodiments provide the gene corresponding to the cDNA sequence of SEQ ID NO:32.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:32, but excluding the poly(A) tail at the 3′ end of SEQ ID NO:32; and
      (ab) the nucleotide sequence of the cDNA insert of clone DA136_{—}11 deposited with the ATCC under accession number 98196;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:32, but excluding the poly(A) tail at the 3′ end of SEQ ID NO:32; and
      (bb) the nucleotide sequence of the cDNA insert of clone DA136_{—}11 deposited with the ATCC under accession number 98196;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:32, and extending contiguously from a nucleotide sequence corresponding to the 5′ end of SEQ ID NO:32 to a nucleotide sequence corresponding to the 3′ end of SEQ ID NO:32, but excluding the poly(A) tail at the 3′ end of SEQ ID NO:32. Also preferably the polynucleotide isolated according to the above process comprises a nucleotide sequence corresponding to the cDNA sequence of SEQ ID NO:32 from nucleotide 338 to nucleotide 1198, and extending contiguously from a nucleotide sequence corresponding to the 5′ end of said sequence of SEQ ID NO:32 from nucleotide 338 to nucleotide 1198, to a nucleotide sequence corresponding to the 3′ end of said sequence of SEQ ID NO:32 from nucleotide 338 to nucleotide 1198. Also preferably the polynucleotide isolated according to the above process comprises a nucleotide sequence corresponding to the cDNA sequence of SEQ ID NO:32 from nucleotide 467 to nucleotide 1058, and extending contiguously from a nucleotide sequence corresponding to the 5′ end of said sequence of SEQ ID NO:32 from nucleotide 467 to nucleotide 1058, to a nucleotide sequence corresponding to the 3′ end of said sequence of SEQ ID NO:32 from nucleotide 467 to nucleotide 1058.

In other embodiments, the present invention provides a composition comprising a protein, wherein said protein comprises an amino acid sequence selected from the group consisting of:
(a) the amino acid sequence of SEQ ID NO:33;
(b) the amino acid sequence of SEQ ID NO:33 from amino acid 124 to amino acid 182;
(c) a fragment of the amino acid sequence of SEQ ID NO:33, the fragment comprising eight contiguous amino acids of SEQ ID NO:33; and
(d) the amino acid sequence encoded by the cDNA insert of clone DA136_{—}11 deposited with the ATCC under accession number 98196;
   the protein being substantially free from other mammalian proteins. Preferably such protein comprises the amino acid sequence of SEQ ID NO:33 or the amino acid sequence of SEQ ID NO:33 from amino acid 124 to amino acid 182. In further preferred embodiments, the present invention provides a protein comprising a fragment of the amino add sequence of SEQ ID NO:33 having biological activity, the fragment preferably comprising eight (more preferably twenty, most preferably thirty) contiguous amino adds of SEQ ID NO:33, or a protein comprising a fragment of the amino acid sequence of SEQ ID NO:33 having biological activity, the fragment comprising the amino acid sequence from amino add 138 to amino acid 147 of SEQ ID NO:33.

In one embodiment, the present invention provides a composition comprising an isolated polynucleotide selected from the group consisting of.
(a) a polynucleotide comprising the nucleotide sequence of SEQ ID NO:34;
(b) a polynucleotide comprising the nucleotide sequence of SEQ ID NO:34 from nucleotide 437 to nucleotide 1159;
(c) a polynucleotide comprising the nucleotide sequence of SEQ ID NO:34 from nucleotide 515 to nucleotide 1159;
(d) a polynucleotide comprising the nucleotide sequence of SEQ ID NO:34 from nucleotide 539 to nucleotide 1099;
(e) a polynucleotide comprising the nucleotide sequence of the full-length protein coding sequence of clone AR415_{—}4 deposited with the ATCC under accession number 98232;
(f) a polynucleotide encoding the full-length protein encoded by the cDNA insert of clone AR415_{—}4 deposited with the ATCC under accession number 98232;
(g) a polynucleotide comprising the nucleotide sequence of a mature protein coding sequence of clone AR415_{—}4 deposited with the ATCC under accession number 98232;
(h) a polynucleotide encoding a mature protein encoded by the cDNA insert of clone AR415_{—}4 deposited with the ATCC under accession number 98232;
(i) a polynucleotide encoding a protein comprising the amino acid sequence of SEQ ID NO:35;
(j) a polynucleotide encoding a protein comprising a fragment of the amino acid sequence of SEQ ID NO:35 having biological activity, the fragment comprising eight contiguous amino acids of SEQ ID NO:35;
(k) a polynucleotide which is an allelic variant of a polynucleotide of (a)-(h) above;
(1) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:34.

Preferably, such polynucleotide comprises the nucleotide sequence of SEQ ID NO:34 from nucleotide 437 to nucleotide 1159; the nucleotide sequence of SEQ ID NO:34 from nucleotide 515 to nucleotide 1159; the nucleotide sequence of SEQ ID NO:34 from nucleotide 539 to nucleotide 1099; the nucleotide sequence of the full-length protein coding sequence of clone AR415_{—}4 deposited with the ATCC under accession number 98232; or the nucleotide sequence of a mature protein coding sequence of clone AR415_{—}4 deposited with the ATCC under accession number 98232. In other preferred embodiments, the polynucleotide encodes the full-length or a mature protein encoded by the cDNA insert of clone AR415_{—}4 deposited with the ATCC under accession number 98232. In yet other preferred embodiments, the present invention provides a polynucleotide encoding a protein comprising the amino acid sequence of SEQ ID NO:35 from amino acid 51 to amino acid 221. In further preferred embodiments, the present invention provides a polynucleotide encoding a protein comprising a fragment of the amino acid sequence of SEQ ID NO:35 having biological activity, the fragment preferably comprising eight (more preferably twenty, most preferably thirty) contiguous amino acids of SEQ ID NO:35, or a polynucleotide encoding a protein comprising a fragment of the amino acid sequence of SEQ ID NO:35 having biological activity, the fragment comprising the amino acid sequence from amino acid 115 to amino acid 124 of SEQ ID NO:35.

Other embodiments provide the gene corresponding to the cDNA sequence of SEQ ID NO:34.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:34, but excluding the poly(A) tail at the 3′ end of SEQ ID NO:34; and
      (ab) the nucleotide sequence of the cDNA insert of clone AR415_{—}4 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of
      (ba) SEQ ID NO:34, but excluding the poly(A) tail at the 3′ end of SEQ ID NO:34; and
      (bb) the nucleotide sequence of the cDNA insert of clone AR415_{—}4 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:34, and extending contiguously from a nucleotide sequence corresponding to the 5′ end of SEQ ID NO:34 to a nucleotide sequence corresponding to the 3′ end of SEQ ID NO:34, but excluding the poly(A) tail at the 3′ end of SEQ ID NO:34. Also preferably the polynucleotide isolated according to the above process comprises a nucleotide sequence corresponding to the cDNA sequence of SEQ ID NO:34 from nucleotide 437 to nucleotide 1159, and extending contiguously from a nucleotide sequence corresponding to the 5′ end of said sequence of SEQ ID NO:34 from nucleotide 437 to nucleotide 1159, to a nucleotide sequence corresponding to the 3′ end of said sequence of SEQ ID NO:34 from nucleotide 437 to nucleotide 1159. Also preferably the polynucleotide isolated according to the above process comprises a nucleotide sequence corresponding to the cDNA sequence of SEQ ID NO:34 from nucleotide 515 to nucleotide 1159, and extending contiguously from a nucleotide sequence corresponding to the 5′ end of said sequence of SEQ ID NO:34 from nucleotide 515 to nucleotide 1159, to a nucleotide sequence corresponding to the 3′ end of said sequence of SEQ ID NO:34 from nucleotide 515 to nucleotide 1159. Also preferably the polynucleotide isolated according to the above process comprises a nucleotide sequence corresponding to the cDNA sequence of SEQ ID NO:34 from nucleotide 539 to nucleotide 1099, and extending contiguously from a nucleotide sequence corresponding to the 5′ end of said sequence of SEQ ID NO:34 from nucleotide 539 to nucleotide 1099, to a nucleotide sequence corresponding to the 3′ end of said sequence of SEQ ID NO:34 from nucleotide 539 to nucleotide 1099.

In other embodiments, the present invention provides a composition comprising a protein, wherein said protein comprises an amino acid sequence selected from the group consisting of:
(a) the amino acid sequence of SEQ ID NO:35;
(b) the amino acid sequence of SEQ ID NO:35 from amino acid 51 to amino acid 221;
(c) a fragment of the amino acid sequence of SEQ ID NO:35, the fragment comprising eight contiguous amino acids of SEQ ID NO:35; and
(d) the amino acid sequence encoded by the cDNA insert of clone AR415_{—}4 deposited with the ATCC under accession number 98232;
   the protein being substantially free from other mammalian proteins. Preferably such protein comprises the amino acid sequence of SEQ ID NO:35 or the amino acid sequence of SEQ ID NO:35 from amino add 51 to amino acid 221. In further preferred embodiments, the present invention provides a protein comprising a fragment of the amino acid sequence of SEQ ID NO:35 having biological activity, the fragment preferably comprising eight (more preferably twenty, most preferably thirty) contiguous amino acids of SEQ ID NO:35, or a protein comprising a fragment of the amino acid sequence of SEQ ID NO:35 having biological activity, the fragment comprising the amino acid sequence from amino acid 115 to amino acid 124 of SEQ ID NO:35.

In one embodiment, the present invention provides a composition comprising an isolated polynucleotide selected from the group consisting of:
(a) a polynucleotide comprising the nucleotide sequence of SEQ ID NO:36;
(b) a polynucleotide comprising the nucleotide sequence of SEQ ID NO:36 from nucleotide 59 to nucleotide 376;
(c) a polynucleotide comprising the nucleotide sequence of SEQ ID NO:36 from nucleotide 179 to nucleotide 376;
(d) a polynucleotide comprising the nucleotide sequence of the full-length protein coding sequence of clone AS63_{—}29 deposited with the ATCC under accession number 98232;
(e) a polynucleotide encoding the full-length protein encoded by the cDNA insert of clone AS63_{—}29 deposited with the ATCC under accession number 98232;
(f) a polynucleotide comprising the nucleotide sequence of a mature protein coding sequence of clone AS63_{—}29 deposited with the ATCC under accession number 98232;
(g) a polynucleotide encoding a mature protein encoded by the cDNA insert of clone AS63_{—}29 deposited with the ATCC under accession number 98232;
(h) a polynucleotide encoding a protein comprising the amino acid sequence of SEQ ID NO:37;
(i) a polynucleotide encoding a protein comprising a fragment of the amino acid sequence of SEQ ID NO:37 having biological activity, the fragment comprising eight contiguous amino adds of SEQ ID NO:37;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:36.

Preferably, such polynucleotide comprises the nucleotide sequence of SEQ ID NO:36 from nucleotide 59 to nucleotide 376; the nucleotide sequence of SEQ ID NO:36 from nucleotide 179 to nucleotide 376; the nucleotide sequence of the full-length protein coding sequence of clone AS63_{—}29 deposited with the ATCC under accession number 98232; or the nucleotide sequence of a mature protein coding sequence of clone AS63_{—}29 deposited with the ATCC under accession number 98232. In other preferred embodiments, the polynucleotide encodes the full-length or a mature protein encoded by the cDNA insert of clone AS63_{—}29 deposited with the ATCC under accession number 98232. In yet other preferred embodiments, the present invention provides a polynucleotide encoding a protein comprising the amino acid sequence of SEQ ID NO:37 from amino acid 1 to amino acid 91. In further preferred embodiments, the present invention provides a polynucleotide encoding a protein comprising a fragment of the amino acid sequence of SEQ ID NO:37 having biological activity, the fragment preferably comprising eight (more preferably twenty, most preferably thirty) contiguous amino acids of SEQ ID NO:37, or a polynucleotide encoding a protein comprising a fragment of the amino acid sequence of SEQ ID NO:37 having biological activity, the fragment comprising the amino acid sequence from amino acid 48 to amino acid 57 of SEQ ID NO:37.

Other embodiments provide the gene corresponding to the cDNA sequence of SEQ ID NO:36 and SEQ ID NO:38.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:36;
      (ab) SEQ ID NO:38, but excluding the poly(A) tail at the 3′ end of SEQ ID NO:38; and
      (ac) the nucleotide sequence of the cDNA insert of clone AS63_{—}29 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:36;
      (bb) SEQ ID NO:38, but excluding the poly(A) tail at the 3′ end of SEQ ID NO:38; and
      (bc) the nucleotide sequence of the cDNA insert of clone AS63_{—}29 deposited with the ATCC under accession number 98232;
(ii) hybridizing said primer(s) to human genomic DNA in conditions at least as stringent as 4×SSC at 50 degrees C.;
(iii) amplifying human DNA sequences; and
(iv) isolating the polynucleotide products of step (b)(iii).
   Preferably the polynucleotide isolated according to the above process comprises a nucleotide sequence corresponding to the cDNA sequences of SEQ ID NO:36 and SEQ ID NO:38, and extending contiguously from a nucleotide sequence corresponding to the 5′ end of SEQ ID NO:36 to a nucleotide sequence corresponding to the 3′ end of SEQ ID NO:38, but excluding the poly(A) tail at the 3′ end of SEQ ID NO:38. Also preferably the polynucleotide isolated according to the above process comprises a nucleotide sequence corresponding to the cDNA sequence of SEQ ID NO:36, and extending contiguously from a nucleotide sequence corresponding to the 5′ end of SEQ ID NO:36 to a nucleotide sequence corresponding to the 3′ end of SEQ ID NO:36. Also preferably the polynucleotide isolated according to the above process comprises a nucleotide sequence corresponding to the cDNA sequence of SEQ ID NO:36 from nucleotide 59 to nucleotide 376, and extending contiguously from a nucleotide sequence corresponding to the 5′ end of said sequence of SEQ ID NO:36 from nucleotide 59 to nucleotide 376, to a nucleotide sequence corresponding to the 3′ end of said sequence of SEQ ID NO:36 from nucleotide 59 to nucleotide 376. Also preferably the polynucleotide isolated according to the above process comprises a nucleotide sequence corresponding to the cDNA sequence of SEQ ID NO:36 from nucleotide 179 to nucleotide 376, and extending contiguously from a nucleotide sequence corresponding to the 5′ end of said sequence of SEQ ID NO:36 from nucleotide 179 to nucleotide 376, to a nucleotide sequence corresponding to the 3′ end of said sequence of SEQ ID NO:36 from nucleotide 179 to nucleotide 376.

In other embodiments, the present invention provides a composition comprising a protein, wherein said protein comprises an amino acid sequence selected from the group consisting of:
(a) the amino acid sequence of SEQ ID NO:37;
(b) the amino acid sequence of SEQ ID NO:37 from amino acid 1 to amino acid 91;
(c) a fragment of the amino acid sequence of SEQ ID NO:37, the fragment comprising eight contiguous amino acids of SEQ ID NO:37; and
(d) the amino acid sequence encoded by the cDNA insert of clone AS63_{—}29 deposited with the ATCC under accession number 98232;
   the protein being substantially free from other mammalian proteins. Preferably such protein comprises the amino acid sequence of SEQ ID NO:37 or the amino acid sequence of SEQ ID NO:37 from amino acid 1 to amino acid 91. In further preferred embodiments, the present invention provides a protein comprising a fragment of the amino acid sequence of SEQ ID NO:37 having biological activity, the fragment preferably comprising eight (more preferably twenty, most preferably thirty) contiguous amino adds of SEQ ID NO:37, or a protein comprising a fragment of the amino acid sequence of SEQ ID NO:37 having biological activity, the fragment comprising the amino acid sequence from amino acid 48 to amino acid 57 of SEQ ID NO:37.

In one embodiment, the present invention provides a composition comprising an isolated polynucleotide selected from the group consisting of:
(a) a polynucleotide comprising the nucleotide sequence of SEQ ID NO:39;
(b) a polynucleotide comprising the nucleotide sequence of SEQ ID NO:39 from nucleotide 198 to nucleotide 2039;
(c) a polynucleotide comprising the nucleotide sequence of SEQ ID NO:39 from nucleotide 490 to nucleotide 809;
(d) a polynucleotide comprising the nucleotide sequence of the full-length protein coding sequence of clone AY304_{—}14 deposited with the ATCC under accession number 98561;
(e) a polynucleotide encoding the full-length protein encoded by the cDNA insert of clone AY304_{—}14 deposited with the ATCC under accession number 98561;
(f) a polynucleotide comprising the nucleotide sequence of a mature protein coding sequence of clone AY304_{—}14 deposited with the ATCC under accession number 98561;
(g) a polynucleotide encoding a mature protein encoded by the cDNA insert of clone AY304_{—}14 deposited with the ATCC under accession number 98561;
(h) a polynucleotide encoding a protein comprising the amino acid sequence of SEQ ID NO:40;
(i) a polynucleotide encoding a protein comprising a fragment of the amino acid sequence of SEQ ID NO:40 having biological activity, the fragment comprising eight contiguous amino acids of SEQ ID NO:40;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:39.

Preferably, such polynucleotide comprises the nucleotide sequence of SEQ ID NO:39 from nucleotide 198 to nucleotide 2039; the nucleotide sequence of SEQ ID NO:39 from nucleotide 490 to nucleotide 809; the nucleotide sequence of the full-length protein coding sequence of clone AY304_{—}14 deposited with the ATCC under accession number 98561; or the nucleotide sequence of a mature protein coding sequence of clone AY304_{—}14 deposited with the ATCC under accession number 98561. In other preferred embodiments, the polynucleotide encodes the full-length or a mature protein encoded by the cDNA insert of clone AY304_{—}14 deposited with the ATCC under accession number 98561. In yet other preferred embodiments, the present invention provides a polynucleotide encoding a protein comprising the amino acid sequence of SEQ ID NO:40 from amino acid 106 to amino acid 204. In further preferred embodiments, the present invention provides a polynucleotide encoding a protein comprising a fragment of the amino acid sequence of SEQ ID NO:40 having biological activity, the fragment preferably comprising eight (more preferably twenty, most preferably thirty) contiguous amino acids of SEQ ID NO:40, or a polynucleotide encoding a protein comprising a fragment of the amino acid sequence of SEQ ID NO:40 having biological activity, the fragment comprising the amino acid sequence from amino acid 302 to amino acid 311 of SEQ ID NO:40.

Other embodiments provide the gene corresponding to the cDNA sequence of SEQ ID NO:39.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:39, but excluding the poly(A) tail at the 3′ end of SEQ ID NO:39; and
      (ab) the nucleotide sequence of the cDNA insert of clone AY304_{—}14 deposited with the ATCC under accession number 98561;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:39, but excluding the poly(A) tail at the 0.3° end of SEQ ID NO:39; and
      (bb) the nucleotide sequence of the cDNA insert of clone AY304_{—}14 deposited with the ATCC under accession number 98561;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:39, and extending contiguously from a nucleotide sequence corresponding to the 5′ end of SEQ ID NO:39 to a nucleotide sequence corresponding to the 3′ end of SEQ ID NO:39, but excluding the poly(A) tail at the 3′ end of SEQ ID NO:39. Also preferably the polynucleotide isolated according to the above process comprises a nucleotide sequence corresponding to the cDNA sequence of SEQ ID NO:39 from nucleotide 198 to nucleotide 2039, and extending contiguously from a nucleotide sequence corresponding to the 5′ end of said sequence of SEQ ID NO:39 from nucleotide 198 to nucleotide 2039, to a nucleotide sequence corresponding to the 3′ end of said sequence of SEQ ID NO:39 from nucleotide 198 to nucleotide 2039. Also preferably the polynucleotide isolated according to the above process comprises a nucleotide sequence corresponding to the cDNA sequence of SEQ ID NO:39 from nucleotide 490 to nucleotide 809, and extending contiguously from a nucleotide sequence corresponding to the 5′ end of said sequence of SEQ ID NO:39 from nucleotide 490 to nucleotide 809, to a nucleotide sequence corresponding to the 3′ end of said sequence of SEQ ID NO:39 from nucleotide 490 to nucleotide 809.

In other embodiments, the present invention provides a composition comprising a protein, wherein said protein comprises an amino add sequence selected from the group consisting of:
(a) the amino acid sequence of SEQ ID NO:40;
(b) the amino add sequence of SEQ ID NO:40 from amino acid 106 to amino acid 204;
(c) a fragment of the amino acid sequence of SEQ ID NO:40, the fragment comprising eight contiguous amino acids of SEQ ID NO:40; and
(d) the amino acid sequence encoded by the cDNA insert of clone AY304_{—}14 deposited with the ATCC under accession number 98561;
   the protein being substantially free from other mammalian proteins. Preferably such protein comprises the amino acid sequence of SEQ ID NO:40 or the amino acid sequence of SEQ ID NO:40 from amino acid 106 to amino acid 204. In further preferred embodiments, the present invention provides a protein comprising a fragment of the amino acid sequence of SEQ ID NO:40 having biological activity, the fragment preferably comprising eight (more preferably twenty, most preferably thirty) contiguous amino acids of SEQ ID NO:40, or a protein comprising a fragment of the amino acid sequence of SEQ ID NO:40 having biological activity, the fragment comprising the amino acid sequence from amino acid 302 to amino acid 311 of SEQ ID NO:40.

In one embodiment, the present invention provides a composition comprising an isolated polynucleotide selected from the group consisting of:
(a) a polynucleotide comprising the nucleotide sequence of SEQ ID NO:41;
(b) a polynucleotide comprising the nucleotide sequence of SEQ ID NO:41 from nucleotide 102 to nucleotide 2027;
(c) a polynucleotide comprising the nucleotide sequence of SEQ ID NO:41 from nucleotide 1902 to nucleotide 2027;
(d) a polynucleotide comprising the nucleotide sequence of SEQ ID NO:41 from nucleotide 1 to nucleotide 431;
(e) a polynucleotide comprising the nucleotide sequence of the full-length protein coding sequence of clone BG160_deposited with the ATCC under accession number 98232;
(f) a polynucleotide encoding the full-length protein encoded by the cDNA insert of clone BG160_{—}1 deposited with the ATCC under accession number 98232;
(g) a polynucleotide comprising the nucleotide sequence of a mature protein coding sequence of clone BG160_deposited with the ATCC under accession number 98232;
(h) a polynucleotide encoding a mature protein encoded by the cDNA insert of clone BG160_{—}1 deposited with the ATCC under accession number 98232;
(i) a polynucleotide encoding a protein comprising the amino acid sequence of SEQ ID NO:42;
(j) a polynucleotide encoding a protein comprising a fragment of the amino acid sequence of SEQ ID NO:42 having biological activity, the fragment comprising eight contiguous amino acids of SEQ ID NO:42;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:41.

Preferably, such polynucleotide comprises the nucleotide sequence of SEQ ID NO:41 from nucleotide 102 to nucleotide 2027; the nucleotide sequence of SEQ ID NO:41 from nucleotide 1902 to nucleotide 2027; the nucleotide sequence of SEQ ID NO:41 from nucleotide 1 to nucleotide 431; the nucleotide sequence of the full-length protein coding sequence of clone BG160_{—}1 deposited with the ATCC under accession number 98232; or the nucleotide sequence of a mature protein coding sequence of clone BG160_{—}1 deposited with the ATCC under accession number 98232. In other preferred embodiments, the polynucleotide encodes the full-length or a mature protein encoded by the cDNA insert of clone BG160_{—}1 deposited with the ATCC under accession number 98232. In yet other preferred embodiments, the present invention provides a polynucleotide encoding a protein comprising the amino acid sequence of SEQ ID NO:42 from amino acid 1 to amino acid 110. In further preferred embodiments, the present invention provides a polynucleotide encoding a protein comprising a fragment of the amino acid sequence of SEQ ID NO:42 having biological activity, the fragment preferably comprising eight (more preferably twenty, most preferably thirty) contiguous amino acids of SEQ ID NO:42, or a polynucleotide encoding a protein comprising a fragment of the amino acid sequence of SEQ ID NO:42 having biological activity, the fragment comprising the amino acid sequence from amino acid 316 to amino acid 325 of SEQ ID NO:42.

Other embodiments provide the gene corresponding to the cDNA sequence of SEQ ID NO:41.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:41, but excluding the poly(A) tail at the 3′ end of SEQ ID NO:41; and
      (ab) the nucleotide sequence of the cDNA insert of clone BG160_C1 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:41, but excluding the poly(A) tail at the 3′ end of SEQ ID NO:41; and
      (bb) the nucleotide sequence of the cDNA insert of clone BG160_{—}1 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:41, and extending contiguously from a nucleotide sequence corresponding to the 5′ end of SEQ ID NO:41 to a nucleotide sequence corresponding to the 3′ end of SEQ ID NO:41, but excluding the poly(A) tail at the 3′ end of SEQ ID NO:41. Also preferably the polynucleotide isolated according to the above process comprises a nucleotide sequence corresponding to the cDNA sequence of SEQ ID NO:41 from nucleotide 102 to nucleotide 2027, and extending contiguously from a nucleotide sequence corresponding to the 5′ end of said sequence of SEQ ID NO:41 from nucleotide 102 to nucleotide 2027, to a nucleotide sequence corresponding to the 3′ end of said sequence of SEQ ID NO:41 from nucleotide 102 to nucleotide 2027. Also preferably the polynucleotide isolated according to the above process comprises a nucleotide sequence corresponding to the cDNA sequence of SEQ ID NO:41 from nucleotide 1902 to nucleotide 2027, and extending contiguously from a nucleotide sequence corresponding to the 5′ end of said sequence of SEQ ID NO:41 from nucleotide 1902 to nucleotide 2027, to a nucleotide sequence corresponding to the 3′ end of said sequence of SEQ ID NO:41 from nucleotide 1902 to nucleotide 2027. Also preferably the polynucleotide isolated according to the above process comprises a nucleotide sequence corresponding to the cDNA sequence of SEQ ID NO:41 from nucleotide 1 to nucleotide 431, and extending contiguously from a nucleotide sequence corresponding to the 5′ end of said sequence of SEQ ID NO:41 from nucleotide 1 to nucleotide 431, to a nucleotide sequence corresponding to the 3′ end of said sequence of SEQ ID NO:41 from nucleotide 1 to nucleotide 431.

In other embodiments, the present invention provides a composition comprising a protein, wherein said protein comprises an amino acid sequence selected from the group consisting of:
(a) the amino acid sequence of SEQ ID NO:42;
(b) the amino acid sequence of SEQ ID NO:42 from amino acid 1 to amino acid 110;
(c) a fragment of the amino acid sequence of SEQ ID NO:42, the fragment comprising eight contiguous amino adds of SEQ ID NO:42; and
(d) the amino acid sequence encoded by the cDNA insert of clone BG160_{—}1 deposited with the ATCC under accession number 98232;
   the protein being substantially free from other mammalian proteins. Preferably such protein comprises the amino acid sequence of SEQ ID NO:42 or the amino add sequence of SEQ ID NO:42 from amino add 1 to amino acid 110. In further preferred embodiments, the present invention provides a protein comprising a fragment of the amino add sequence of SEQ ID NO:42 having biological activity, the fragment preferably comprising eight (more preferably twenty, most preferably thirty) contiguous amino acids of SEQ ID NO:42, or a protein comprising a fragment of the amino acid sequence of SEQ ID NO:42 having biological activity, the fragment comprising the amino add sequence from amino add 316 to amino add 325 of SEQ ID NO:42.

In one embodiment, the present invention provides a composition comprising an isolated polynucleotide selected from the group consisting of:
(a) a polynucleotide comprising the nucleotide sequence of SEQ ID NO:44;
(b) a polynucleotide comprising the nucleotide sequence of SEQ ID NO:44 from nucleotide 566 to nucleotide 631;
(c) a polynucleotide comprising the nucleotide sequence of the full-length protein coding sequence of clone BO432_{—}4 deposited with the ATCC under accession number 98232;
(d) a polynucleotide encoding the full-length protein encoded by the cDNA insert of clone BO432_{—}4 deposited with the ATCC under accession number 98232;
(e) a polynucleotide comprising the nucleotide sequence of a mature protein coding sequence of clone BO432_{—}4 deposited with the ATCC under accession number 98232;
(f) a polynucleotide encoding a mature protein encoded by the cDNA insert of clone BO432_{—}4 deposited with the ATCC under accession number 98232;
(g) a polynucleotide encoding a protein comprising the amino acid sequence of SEQ ID NO:45;
(h) a polynucleotide encoding a protein comprising a fragment of the amino acid sequence of SEQ ID NO:45 having biological activity, the fragment comprising eight contiguous amino acids of SEQ ID NO:45;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:44.

Preferably, such polynucleotide comprises the nucleotide sequence of SEQ ID NO:44 from nucleotide 566 to nucleotide 631; the nucleotide sequence of the full-length protein coding sequence of clone BO432_{—}4 deposited with the ATCC under accession number 98232; or the nucleotide sequence of a mature protein coding sequence of clone BO432_{—}4 deposited with the ATCC under accession number 98232. In other preferred embodiments, the polynucleotide encodes the full-length or a mature protein encoded by the cDNA insert of clone BO432_{—}4 deposited with the ATCC under accession number 98232. In further preferred embodiments, the present invention provides a polynucleotide encoding a protein comprising a fragment of the amino acid sequence of SEQ ID NO:45 having biological activity, the fragment preferably comprising eight (more preferably twenty, most preferably thirty) contiguous amino acids of SEQ ID NO:45, or a polynucleotide encoding a protein comprising a fragment of the amino acid sequence of SEQ ID NO:45 having biological activity, the fragment comprising the amino acid sequence from amino acid 6 to amino acid 15 of SEQ ID NO:45.

Other embodiments provide the gene corresponding to the cDNA sequence of SEQ ID NO:44, SEQ ID NO:43, and SEQ ID NO:46.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:43;
      (ab) SEQ ID NO:44;
      (ac) SEQ ID NO:46, but excluding the poly(A) tail at the 3′ end of SEQ ID NO:46; and
      (ad) the nucleotide sequence of the cDNA insert of clone BO432_{—}4 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:43;
      (bb) SEQ ID NO:44;
      (bc) SEQ ID NO:46, but excluding the poly(A) tail at the 3′ end of SEQ ID NO:46; and
      (bd) the nucleotide sequence of the cDNA insert of clone BO432_{—}4 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:43, SEQ ID NO:44, and SEQ ID NO:46, and extending contiguously from a nucleotide sequence corresponding to the 5′ end of SEQ ID NO:43 to a nucleotide sequence corresponding to the 3′ end of SEQ ID NO:46, but excluding the poly(A) tail at the 3′ end of SEQ ID NO:46. Also preferably the polynucleotide isolated according to the above process comprises a nucleotide sequence corresponding to the cDNA sequence of SEQ ID NO:44, and extending contiguously from a nucleotide sequence corresponding to the 5′ end of SEQ ID NO:44 to a nucleotide sequence corresponding to the 3′ end of SEQ ID NO:44. Also preferably the polynucleotide isolated according to the above process comprises a nucleotide sequence corresponding to the cDNA sequence of SEQ ID NO:44 from nucleotide 566 to nucleotide 631, and extending contiguously from a nucleotide sequence corresponding to the 5′ end of said sequence of SEQ ID NO:44 from nucleotide 566 to nucleotide 631, to a nucleotide sequence corresponding to the 3′ end of said sequence of SEQ ID NO:44 from nucleotide 566 to nucleotide 631.

In other embodiments, the present invention provides a composition comprising a protein, wherein said protein comprises an amino acid sequence selected from the group consisting of:
(a) the amino acid sequence of SEQ ID NO:45;
(b) a fragment of the amino acid sequence of SEQ ID NO:45, the fragment comprising eight contiguous amino acids of SEQ ID NO:45; and
(c) the amino acid sequence encoded by the cDNA insert of clone BO432_{—}4 deposited with the ATCC under accession number 98232;
   the protein being substantially free from other mammalian proteins. Preferably such protein comprises the amino acid sequence of SEQ ID NO:45. In further preferred embodiments, the present invention provides a protein comprising a fragment of the amino acid sequence of SEQ ID NO:45 having biological activity, the fragment preferably comprising eight (more preferably twenty, most preferably thirty) contiguous amino acids of SEQ ID NO:45, or a protein comprising a fragment of the amino acid sequence of SEQ ID NO:45 having biological activity, the fragment comprising the amino acid sequence from amino acid 6 to amino acid 15 of SEQ ID NO:45.

In one embodiment, the present invention provides a composition comprising an isolated polynucleotide selected from the group consisting of:
(a) a polynucleotide comprising the nucleotide sequence of SEQ ID NO:47;
(b) a polynucleotide comprising the nucleotide sequence of SEQ ID NO:47 from nucleotide 45 to nucleotide 428;
(c) a polynucleotide comprising the nucleotide sequence of the full-length protein coding sequence of clone BO538_{—}2 deposited with the ATCC under accession number 98232;
(d) a polynucleotide encoding the full-length protein encoded by the cDNA insert of clone BO538_{—}2 deposited with the ATCC under accession number 98232;
(e) a polynucleotide comprising the nucleotide sequence of a mature protein coding sequence of clone BO538_{—}2 deposited with the ATCC under accession number 98232;
(f) a polynucleotide encoding a mature protein encoded by the cDNA insert of clone BO538_{—}2 deposited with the ATCC under accession number 98232;
(g) a polynucleotide encoding a protein comprising the amino acid sequence of SEQ ID NO:48;
(h) a polynucleotide encoding a protein comprising a fragment of the amino acid sequence of SEQ ID NO:48 having biological activity, the fragment comprising eight contiguous amino acids of SEQ ID NO:48;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:47.

Preferably, such polynucleotide comprises the nucleotide sequence of SEQ ID NO:47 from nucleotide 45 to nucleotide 428; the nucleotide sequence of the full-length protein coding sequence of clone BO538_{—}2 deposited with the ATCC under accession number 98232; or the nucleotide sequence of a mature protein coding sequence of clone BO538_{—}2 deposited with the ATCC under accession number 98232. In other preferred embodiments, the polynucleotide encodes the full-length or a mature protein encoded by the cDNA insert of clone BO538_{—}0.2 deposited with the ATCC under accession number 98232. In yet other preferred embodiments, the present invention provides a polynucleotide encoding a protein comprising the amino acid sequence of SEQ ID NO:48 from amino acid 52 to amino acid 128. In further preferred embodiments, the present invention provides a polynucleotide encoding a protein comprising a fragment of the amino acid sequence of SEQ ID NO:48 having biological activity, the fragment preferably comprising eight (more preferably twenty, most preferably thirty) contiguous amino acids of SEQ ID NO:48, or a polynucleotide encoding a protein comprising a fragment of the amino acid sequence of SEQ ID NO:48 having biological activity, the fragment comprising the amino acid sequence from amino acid 59 to amino acid 68 of SEQ ID NO:48.

Other embodiments provide the gene corresponding to the cDNA sequence of SEQ ID NO:47 and SEQ ID NO:49.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:47;
      (ab) SEQ ID NO:49, but excluding the poly(A) tail at the 3′ end of SEQ ID NO:49; and
      (ac) the nucleotide sequence of the cDNA insert of clone BO538_{—}2 deposited with the ATCC under accession number 98232;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:47;
      (bb) SEQ ID NO:49, but excluding the poly(A) tail at the 3′ end of SEQ ID NO:49; and
      (bc) the nucleotide sequence of the cDNA insert of clone BO538_{—}2 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:47 and SEQ ID NO:49, and extending contiguously from a nucleotide sequence corresponding to the 5′ end of SEQ ID NO:47 to a nucleotide sequence corresponding to the 3′ end of SEQ ID NO:49, but excluding the poly(A) tail at the 3′ end of SEQ ID NO:49. Also preferably the polynucleotide isolated according to the above process comprises a nucleotide sequence corresponding to the cDNA sequence of SEQ ID NO:47, and extending contiguously from a nucleotide sequence corresponding to the 5′ end of SEQ ID NO:47 to a nucleotide sequence corresponding to the 3′ end of SEQ ID NO:47. Also preferably the polynucleotide isolated according to the above process comprises a nucleotide sequence corresponding to the cDNA sequence of SEQ ID NO:47 from nucleotide 45 to nucleotide 428, and extending contiguously from a nucleotide sequence corresponding to the 5′ end of said sequence of SEQ ID NO:47 from nucleotide 45 to nucleotide 428, to a nucleotide sequence corresponding to the 3′ end of said sequence of SEQ ID NO:47 from nucleotide 45 to nucleotide 428.

In other embodiments, the present invention provides a composition comprising a protein, wherein said protein comprises an amino acid sequence selected from the group consisting of
(a) the amino acid sequence of SEQ ID NO:48;
(b) the amino add sequence of SEQ ID NO:48 from amino add 52 to amino add 128;
(c) a fragment of the amino acid sequence of SEQ ID NO:48, the fragment comprising eight contiguous amino acids of SEQ ID NO:48; and
(d) the amino add sequence encoded by the cDNA insert of clone BO538_{—}2 deposited with the ATCC under accession number 98232;
   the protein being substantially free from other mammalian proteins. Preferably such protein comprises the amino add sequence of SEQ ID NO:48 or the amino add sequence of SEQ ID NO:48 from amino acid 52 to amino acid 128. In further preferred embodiments, the present invention provides a protein comprising a fragment of the amino acid sequence of SEQ ID NO:48 having biological activity, the fragment preferably comprising eight (more preferably twenty, most preferably thirty) contiguous amino adds of SEQ ID NO:48, or a protein comprising a fragment of the amino add sequence of SEQ ID NO:48 having biological activity, the fragment comprising the amino acid sequence from amino add 59 to amino acid 68 of SEQ ID NO:48.

In one embodiment, the present invention provides a composition comprising an isolated polynucleotide selected from the group consisting of:
(a) a polynucleotide comprising the nucleotide sequence of SEQ ID NO:50;
(b) a polynucleotide comprising the nucleotide sequence of SEQ ID NO:50 from nucleotide 144 to nucleotide 566;
(c) a polynucleotide comprising the nucleotide sequence of the full-length protein coding sequence of clone BR595_{—}4 deposited with the ATCC under accession number 98232;
(d) a polynucleotide encoding the full-length protein encoded by the cDNA insert of clone BR95_{—}4 deposited with the ATCC under accession number 98232;
(e) a polynucleotide comprising the nucleotide sequence of a mature protein coding sequence of clone BR595_{—}4 deposited with the ATCC under accession number 98232;
(f) a polynucleotide encoding a mature protein encoded by the cDNA insert of clone BR595_{—}4 deposited with the ATCC under accession number 98232;
(g) a polynucleotide encoding a protein comprising the amino acid sequence of SEQ ID NO:51;
(h) a polynucleotide encoding a protein comprising a fragment of the amino acid sequence of SEQ ID NO:51 having biological activity, the fragment comprising eight contiguous amino acids of SEQ ID NO:51;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:50.

Preferably, such polynucleotide comprises the nucleotide sequence of SEQ ID NO:50 from nucleotide 144 to nucleotide 566; the nucleotide sequence of the full-length protein coding sequence of clone BR595_{—}4 deposited with the ATCC under accession number 98232; or the nucleotide sequence of a mature protein coding sequence of clone BR595_{—}4 deposited with the ATCC under accession number 98232. In other preferred embodiments, the polynucleotide encodes the full-length or a mature protein encoded by the cDNA insert of clone BR595_{—}4 deposited with the ATCC under accession number 98232. In yet other preferred embodiments, the present invention provides a polynucleotide encoding a protein comprising the amino acid sequence of SEQ ID NO:51 from amino acid 39 to amino acid 141. In further preferred embodiments, the present invention provides a polynucleotide encoding a protein comprising a fragment of the amino acid sequence of SEQ ID NO:51 having biological activity, the fragment preferably comprising eight (more preferably twenty, most preferably thirty) contiguous amino adds of SEQ ID NO:51, or a polynucleotide encoding a protein comprising a fragment of the amino acid sequence of SEQ ID NO:51 having biological activity, the fragment comprising the amino acid sequence from amino acid 65 to amino acid 74 of SEQ ID NO:51.

Other embodiments provide the gene corresponding to the cDNA sequence of SEQ ID NO:50 and SEQ ID NO:52.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:50;
      (ab) SEQ ID NO:52, but excluding the poly(A) tail at the 3′ end of SEQ ID NO:52; and
      (ac) the nucleotide sequence of the cDNA insert of clone BR595_{—}4 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:50;
      (bb) SEQ ID NO:52, but excluding the poly(A) tail at the 3′ end of SEQ ID NO:52; and
      (bc) the nucleotide sequence of the cDNA insert of clone BR595_{—}4 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:50 and SEQ ID NO:52, and extending contiguously from a nucleotide sequence corresponding to the 5′ end of SEQ ID NO:50 to a nucleotide sequence corresponding to the 3′ end of SEQ ID NO:52, but excluding the poly(A) tail at the 3′ end of SEQ ID NO:52. Also preferably the polynucleotide isolated according to the above process comprises a nucleotide sequence corresponding to the cDNA sequence of SEQ ID NO:50, and extending contiguously from a nucleotide sequence corresponding to the 5′ end of SEQ ID NO:50 to a nucleotide sequence corresponding to the 3′ end of SEQ ID NO:50. Also preferably the polynucleotide isolated according to the above process comprises a nucleotide sequence corresponding to the cDNA sequence of SEQ ID NO:50 from nucleotide 144 to nucleotide 566, and extending contiguously from a nucleotide sequence corresponding to the 5′ end of said sequence of SEQ ID NO:50 from nucleotide 144 to nucleotide 566, to a nucleotide sequence corresponding to the 3′ end of said sequence of SEQ ID NO:50 from nucleotide 144 to nucleotide 566.

In other embodiments, the present invention provides a composition comprising a protein, wherein said protein comprises an amino acid sequence selected from the group consisting of:
(a) the amino acid sequence of SEQ ID NO:51;
(b) the amino acid sequence of SEQ ID NO:51 from amino acid 39 to amino acid 141;
(c) a fragment of the amino acid sequence of SEQ ID NO:51, the fragment comprising eight contiguous amino acids of SEQ ID NO:51; and
(d) the amino acid sequence encoded by the cDNA insert of clone BR595_{—}4 deposited with the ATCC under accession number 98232;
   the protein being substantially free from other mammalian proteins. Preferably such protein comprises the amino add sequence of SEQ ID NO:51 or the amino acid sequence of SEQ ID NO:51 from amino acid 39 to amino acid 141. In further preferred embodiments, the present invention provides a protein comprising a fragment of the amino acid sequence of SEQ ID NO:51 having biological activity, the fragment preferably comprising eight (more preferably twenty, most preferably thirty) contiguous amino adds of SEQ ID NO:51, or a protein comprising a fragment of the amino acid sequence of SEQ ID NO:51 having biological activity, the fragment comprising the amino acid sequence from amino acid 65 to amino acid 74 of SEQ ID NO:51.

In one embodiment, the present invention provides a composition comprising an isolated polynucleotide selected from the group consisting of:
(a) a polynucleotide comprising the nucleotide sequence of SEQ ID NO:53;
(b) a polynucleotide comprising the nucleotide sequence of SEQ ID NO:53 from nucleotide 232 to nucleotide 1041;
(c) a polynucleotide comprising the nucleotide sequence of SEQ ID NO:53 from nucleotide 460 to nucleotide 1041;
(d) a polynucleotide comprising the nucleotide sequence of SEQ ID NO:53 from nucleotide 590 to nucleotide 1163;
(e) a polynucleotide comprising the nucleotide sequence of the full-length protein coding sequence of clone CI490_{—}2 deposited with the ATCC under accession number 98232;
(f) a polynucleotide encoding the full-length protein encoded by the cDNA insert of clone CI490_{—}2 deposited with the ATCC under accession number 98232;
(g) a polynucleotide comprising the nucleotide sequence of a mature protein coding sequence of clone CI490_{—}2 deposited with the ATCC under accession number 98232;
(h) a polynucleotide encoding a mature protein encoded by the cDNA insert of clone CI490_{—}2 deposited with the ATCC under accession number 98232;
(i) a polynucleotide encoding a protein comprising the amino acid sequence of SEQ ID NO:54;
(j) a polynucleotide encoding a protein comprising a fragment of the amino acid sequence of SEQ ID NO:54 having biological activity, the fragment comprising eight contiguous amino acids of SEQ ID NO:54;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:53.

Preferably, such polynucleotide comprises the nucleotide sequence of SEQ ID NO:53 from nucleotide 232 to nucleotide 1041; the nucleotide sequence of SEQ ID NO:53 from nucleotide 460 to nucleotide 1041; the nucleotide sequence of SEQ ID NO:53 from nucleotide 590 to nucleotide 1163; the nucleotide sequence of the full-length protein coding sequence of clone CI490_{—}2 deposited with the ATCC under accession number 98232; or the nucleotide sequence of a mature protein coding sequence of clone CI490_{—}2 deposited with the ATCC under accession number 98232. In other preferred embodiments, the polynucleotide encodes the full-length or a mature protein encoded by the cDNA insert of clone CI490_{—}2 deposited with the ATCC under accession number 98232. In yet other preferred embodiments, the present invention provides a polynucleotide encoding a protein comprising the amino acid sequence of SEQ ID NO:54 from amino acid 133 to amino acid 270. In further preferred embodiments, the present invention provides a polynucleotide encoding a protein comprising a fragment of the amino acid sequence of SEQ ID NO:54 having biological activity, the fragment preferably comprising eight (more preferably twenty, most preferably thirty) contiguous amino acids of SEQ ID NO:54, or a polynucleotide encoding a protein comprising a fragment of the amino add sequence of SEQ ID NO:54 having biological activity, the fragment comprising the amino acid sequence from amino acid 130 to amino acid 139 of SEQ ID NO:54.

Other embodiments provide the gene corresponding to the cDNA sequence of SEQ ID NO:53.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:53, but excluding the poly(A) tail at the 3′ end of SEQ ID NO:53; and
      (ab) the nucleotide sequence of the cDNA insert of clone CI490_{—}2 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:53, but excluding the poly(A) tail at the 3′ end of SEQ ID NO:53; and
      (bb) the nucleotide sequence of the cDNA insert of clone CI490_{—}2 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:53, and extending contiguously from a nucleotide sequence corresponding to the 5′ end of SEQ ID NO:53 to a nucleotide sequence corresponding to the 3′ end of SEQ ID NO:53, but excluding the poly(A) tail at the 3′ end of SEQ ID NO:53. Also preferably the polynucleotide isolated according to the above process comprises a nucleotide sequence corresponding to the cDNA sequence of SEQ ID NO:53 from nucleotide 232 to nucleotide 1041, and extending contiguously from a nucleotide sequence corresponding to the 5′ end of said sequence of SEQ ID NO:53 from nucleotide 232 to nucleotide 1041, to a nucleotide sequence corresponding to the 3′ end of said sequence of SEQ ID NO:53 from nucleotide 232 to nucleotide 1041. Also preferably the polynucleotide isolated according to the above process comprises a nucleotide sequence corresponding to the cDNA sequence of SEQ ID NO:53 from nucleotide 460 to nucleotide 1041, and extending contiguously from a nucleotide sequence corresponding to the 5′ end of said sequence of SEQ ID NO:53 from nucleotide 460 to nucleotide 1041, to a nucleotide sequence corresponding to the 3′ end of said sequence of SEQ ID NO:53 from nucleotide 460 to nucleotide 1041. Also preferably the polynucleotide isolated according to the above process comprises a nucleotide sequence corresponding to the cDNA sequence of SEQ ID NO:53 from nucleotide 590 to nucleotide 1163, and extending contiguously from a nucleotide sequence corresponding to the 5′ end of said sequence of SEQ ID NO:53 from nucleotide 590 to nucleotide 1163, to a nucleotide sequence corresponding to the 3′ end of said sequence of SEQ ID NO:53 from nucleotide 590 to nucleotide 1163.

In other embodiments, the present invention provides a composition comprising a protein, wherein said protein comprises an amino acid sequence selected from the group consisting of:
(a) the amino acid sequence of SEQ ID NO:54;
(b) the amino acid sequence of SEQ ID NO:54 from amino acid 133 to amino acid 270;
(c) a fragment of the amino acid sequence of SEQ ID NO:54, the fragment comprising eight contiguous amino acids of SEQ ID NO:54; and
(d) the amino acid sequence encoded by the cDNA insert of clone CI490_{—}2 deposited with the ATCC under accession number 98232;
   the protein being substantially free from other mammalian proteins. Preferably such protein comprises the amino acid sequence of SEQ ID NO:54 or the amino acid sequence of SEQ ID NO:54 from amino acid 133 to amino acid 270. In further preferred embodiments, the present invention provides a protein comprising a fragment of the amino acid sequence of SEQ ID NO:54 having biological activity, the fragment preferably comprising eight (more preferably twenty, most preferably thirty) contiguous amino acids of SEQ ID NO:54, or a protein comprising a fragment of the amino acid sequence of SEQ ID NO:54 having biological activity, the fragment comprising the amino acid sequence from amino acid 130 to amino acid 139 of SEQ ID NO:54.

In one embodiment, the present invention provides a composition comprising an isolated polynucleotide selected from the group consisting of:
(a) a polynucleotide comprising the nucleotide sequence of SEQ ID NO:55;
(b) a polynucleotide comprising the nucleotide sequence of SEQ ID NO:55 from nucleotide 268 to nucleotide 624;
(c) a polynucleotide comprising the nucleotide sequence of SEQ ID NO:55 from nucleotide 325 to nucleotide 624;
(d) a polynucleotide comprising the nucleotide sequence of the full-length protein coding sequence of clone CI522_{—}1 deposited with the ATCC under accession number 98232;
(e) a polynucleotide encoding the full-length protein encoded by the cDNA insert of clone CI522_{—}1 deposited with the ATCC under accession number 98232;
(f) a polynucleotide comprising the nucleotide sequence of a mature protein coding sequence of clone CI522_{—}1 deposited with the ATCC under accession number 98232;
(g) a polynucleotide encoding a mature protein encoded by the cDNA insert of clone CI522_{—}1 deposited with the ATCC under accession number 98232;
(h) a polynucleotide encoding a protein comprising the amino acid sequence of SEQ ID NO:56;
(i) a polynucleotide encoding a protein comprising a fragment of the amino acid sequence of SEQ ID NO:56 having biological activity, the fragment comprising eight contiguous amino acids of SEQ ID NO:56;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:55.

Preferably, such polynucleotide comprises the nucleotide sequence of SEQ ID NO:55 from nucleotide 268 to nucleotide 624; the nucleotide sequence of SEQ ID NO:55 from nucleotide 325 to nucleotide 624; the nucleotide sequence of the full-length protein coding sequence of clone CI522_{—}1 deposited with the ATCC under accession number 98232; or the nucleotide sequence of a mature protein coding sequence of clone CI522_{—}1 deposited with the ATCC under accession number 98232. In other preferred embodiments, the polynucleotide encodes the full-length or a mature protein encoded by the cDNA insert of clone CI522_{—}1 deposited with the ATCC under accession number 98232. In further preferred embodiments, the present invention provides a polynucleotide encoding a protein comprising a fragment of the amino acid sequence of SEQ ID. NO:56 having biological activity, the fragment preferably comprising eight (more preferably twenty, most preferably thirty) contiguous amino acids of SEQ ID NO:56, or a polynucleotide encoding a protein comprising a fragment of the amino acid sequence of SEQ ID NO:56 having biological activity, the fragment comprising the amino acid sequence from amino acid 54 to amino acid 63 of SEQ ID NO:56.

Other embodiments provide the gene corresponding to the cDNA sequence of SEQ ID NO:55 and SEQ ID NO:57.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:55;
      (ab) SEQ ID NO:57, but excluding the poly(A) tail at the 3′ end of SEQ ID NO:57; and
      (ac) the nucleotide sequence of the cDNA insert of clone CI522_{—}1 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:55;
      (bb) SEQ ID NO:57, but excluding the poly(A) tail at the 3′ end of SEQ ID NO:57; and
      (bc) the nucleotide sequence of the cDNA insert of clone CI5221 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:55 and SEQ ID NO:57, and extending contiguously from a nucleotide sequence corresponding to the 5′ end of SEQ ID NO:55 to a nucleotide sequence corresponding to the 3′ end of SEQ ID NO:57, but excluding the poly(A) tail at the 3′ end of SEQ ID NO:57. Also preferably the polynucleotide isolated according to the above process comprises a nucleotide sequence corresponding to the cDNA sequence of SEQ ID NO:55, and extending contiguously from a nucleotide sequence corresponding to the 5′ end of SEQ ID NO:55 to a nucleotide sequence corresponding to the 3′ end of SEQ ID NO:55. Also preferably the polynucleotide isolated according to the above process comprises a nucleotide sequence corresponding to the cDNA sequence of SEQ ID NO:55 from nucleotide 268 to nucleotide 624, and extending contiguously from a nucleotide sequence corresponding to the 5′ end of said sequence of SEQ ID NO:55 from nucleotide 268 to nucleotide 624, to a nucleotide sequence corresponding to the 3′ end of said sequence of SEQ ID NO:55 from nucleotide 268 to nucleotide 624. Also preferably the polynucleotide isolated according to the above process comprises a nucleotide sequence corresponding to the cDNA sequence of SEQ ID NO:55 from nucleotide 325 to nucleotide 624, and extending contiguously from a nucleotide sequence corresponding to the 5′ end of said sequence of SEQ ID NO:55 from nucleotide 325 to nucleotide 624, to a nucleotide sequence corresponding to the 3′ end of said sequence of SEQ ID NO:55 from nucleotide 325 to nucleotide 624.

In other embodiments, the present invention provides a composition comprising a protein, wherein said protein comprises an amino acid sequence selected from the group consisting of:
(a) the amino acid sequence of SEQ ID NO:56;
(b) a fragment of the amino acid sequence of SEQ ID NO:56, the fragment comprising eight contiguous amino acids of SEQ ID NO:56; and
(c) the amino acid sequence encoded by the cDNA insert of clone CI522_{—}1 deposited with the ATCC under accession number 98232;
   the protein being substantially free from other mammalian proteins. Preferably such protein comprises the amino acid sequence of SEQ ID NO:56. In further preferred embodiments, the present invention provides a protein comprising a fragment of the amino acid sequence of SEQ ID NO:56 having biological activity, the fragment preferably comprising eight (more preferably twenty, most preferably thirty) contiguous amino acids of SEQ ID NO:56, or a protein comprising a fragment of the amino acid sequence of SEQ ID NO:56 having biological activity, the fragment comprising the amino acid sequence from amino acid 54 to amino acid 63 of SEQ ID NO:56.

In one embodiment, the present invention provides a composition comprising an isolated polynucleotide selected from the group consisting of:
(a) a polynucleotide comprising the nucleotide sequence of SEQ ID NO:58;
(b) a polynucleotide comprising the nucleotide sequence of SEQ ID NO:58 from nucleotide 288 to nucleotide 710;
(c) a polynucleotide comprising the nucleotide sequence of SEQ ID NO:58 from nucleotide 868 to nucleotide 1887;
(d) a polynucleotide comprising the nucleotide sequence of the full-length protein coding sequence of clone CN238_{—}1 deposited with the ATCC under accession number 98232;
(e) a polynucleotide encoding the full-length protein encoded by the cDNA insert of clone CN238_{—}1 deposited with the ATCC under accession number 98232;
(f) a polynucleotide comprising the nucleotide sequence of a mature protein coding sequence of clone CN238_{—}1 deposited with the ATCC under accession number 98232;
(g) a polynucleotide encoding a mature protein encoded by the cDNA insert of clone CN238_{—}1 deposited with the ATCC under accession number 98232;
(h) a polynucleotide encoding a protein comprising the amino acid sequence of SEQ ID NO:59;
(i) a polynucleotide encoding a protein comprising a fragment of the amino acid sequence of SEQ ID NO:59 having biological activity, the fragment comprising eight contiguous amino acids of SEQ ID NO:59;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:58.

Preferably, such polynucleotide comprises the nucleotide sequence of SEQ ID NO:58 from nucleotide 288 to nucleotide 710; the nucleotide sequence of SEQ ID NO:58 from nucleotide 868 to nucleotide 1887; the nucleotide sequence of the full-length protein coding sequence of clone CN238_{—}1 deposited with the ATCC under accession number 98232; or the nucleotide sequence of a mature protein coding sequence of clone CN238_{—}1 deposited with the ATCC under accession number 98232. In other preferred embodiments, the polynucleotide encodes the full-length or a mature protein encoded by the cDNA insert of clone CN238_{—}1 deposited with the ATCC under accession number 98232. In yet other preferred embodiments, the present invention provides a polynucleotide encoding a protein comprising the amino acid sequence of SEQ ID NO:59 from amino acid 1 to amino add 109. In further preferred embodiments, the present invention provides a polynucleotide encoding a protein comprising a fragment of the amino acid sequence of SEQ ID NO:59 having biological activity, the fragment preferably comprising eight (more preferably twenty, most preferably thirty) contiguous amino acids of SEQ ID NO:59, or a polynucleotide encoding a protein comprising a fragment of the amino acid sequence of SEQ ID NO:59 having biological activity, the fragment comprising the amino acid sequence from amino acid 65 to amino add 74 of SEQ ID NO:59.

Other embodiments provide the gene corresponding to the cDNA sequence of SEQ ID NO:58.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:58, but excluding the poly(A) tail at the 3′ end of SEQ ID NO:58; and
      (ab) the nucleotide sequence of the cDNA insert of clone CN238_{—}1 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:58, but excluding the poly(A) tail at the 3′ end of SEQ ID NO:58; and
      (bb) the nucleotide sequence of the cDNA insert of clone CN238_{—}1 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:58, and extending contiguously from a nucleotide sequence corresponding to the 5′ end of SEQ ID NO:58 to a nucleotide sequence corresponding to the 3′ end of SEQ ID NO:58, but excluding the poly(A) tail at the 3′ end of SEQ ID NO:58. Also preferably the polynucleotide isolated according to the above process comprises a nucleotide sequence corresponding to the cDNA sequence of SEQ ID NO:58 from nucleotide 288 to nucleotide 710, and extending contiguously from a nucleotide sequence corresponding to the 5′ end of said sequence of SEQ ID NO:58 from nucleotide 288 to nucleotide 710, to a nucleotide sequence corresponding to the 3′ end of said sequence of SEQ ID NO:58 from nucleotide 288 to nucleotide 710. Also preferably the polynucleotide isolated according to the above process comprises a nucleotide sequence corresponding to the cDNA sequence of SEQ ID NO:58 from nucleotide 868 to nucleotide 1887, and extending contiguously from a nucleotide sequence corresponding to the 5′ end of said sequence of SEQ ID NO:58 from nucleotide 868 to nucleotide 1887, to a nucleotide sequence corresponding to the 3′ end of said sequence of SEQ ID. NO:58 from nucleotide 868 to nucleotide 1887.

In other embodiments, the present invention provides a composition comprising a protein, wherein said protein comprises an amino acid sequence selected from the group consisting of:
(a) the amino acid sequence of SEQ ID NO:59;
(b) the amino acid sequence of SEQ ID NO:59 from amino acid 1 to amino acid 109;
(c) a fragment of the amino acid sequence of SEQ ID NO:59, the fragment comprising eight contiguous amino acids of SEQ ID NO:59; and
(d) the amino acid sequence encoded by the cDNA insert of clone CN238_{—}1 deposited with the ATCC under accession number 98232;
   the protein being substantially free from other mammalian proteins. Preferably such protein comprises the amino acid sequence of SEQ ID NO:59 or the amino acid sequence of SEQ ID NO:59 from amino acid 1 to amino acid 109. In further preferred embodiments, the present invention provides a protein comprising a fragment of the amino acid sequence of SEQ ID NO:59 having biological activity, the fragment preferably comprising eight (more preferably twenty, most preferably thirty) contiguous amino acids of SEQ ID NO:59, or a protein comprising a fragment of the amino acid sequence of SEQ ID NO:59 having biological activity, the fragment comprising the amino acid sequence from amino acid 65 to amino acid 74 of SEQ ID NO:59.

In one embodiment, the present invention provides a composition comprising an isolated polynucleotide selected from the group consisting of:
(a) a polynucleotide comprising the nucleotide sequence of SEQ ID NO:60;
(b) a polynucleotide comprising the nucleotide sequence of SEQ ID NO:60 from nucleotide 87 to nucleotide 1871;
(c) a polynucleotide comprising the nucleotide sequence of SEQ ID NO:60 from nucleotide 628 to nucleotide 1882;
(d) a polynucleotide comprising the nucleotide sequence of the full-length protein coding sequence of clone CO390_{—}1 deposited with the ATCC under accession number 98232;
(e) a polynucleotide encoding the full-length protein encoded by the cDNA insert of clone CO390_{—}1 deposited with the ATCC under accession number 98232;
(f) a polynucleotide comprising the nucleotide sequence of a mature protein coding sequence of clone CO390_{—}1 deposited with the ATCC under accession number 98232;
(g) a polynucleotide encoding a mature protein encoded by the cDNA insert of clone CO390_{—}1 deposited with the ATCC under accession number 98232;
(h) a polynucleotide encoding a protein comprising the amino add sequence of SEQ ID NO:61;
(i) a polynucleotide encoding a protein comprising a fragment of the amino add sequence of SEQ ID NO:61 having biological activity, the fragment comprising eight contiguous amino acids of SEQ ID NO:61;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:60.

Preferably, such polynucleotide comprises the nucleotide sequence of SEQ ID NO:60 from nucleotide 87 to nucleotide 1871; the nucleotide sequence of SEQ ID NO:60 from nucleotide 628 to nucleotide 1882; the nucleotide sequence of the full-length protein coding sequence of clone CO390_{—}1 deposited with the ATCC under accession number 98232; or the nucleotide sequence of a mature protein coding sequence of clone CO390_{—}1 deposited with the ATCC under accession number 98232. In other preferred embodiments, the polynucleotide encodes the full-length or a mature protein encoded by the cDNA insert of clone CO390_{—}1 deposited with the ATCC under accession number 98232. In yet other preferred embodiments, the present invention provides a polynucleotide encoding a protein comprising the amino acid sequence of SEQ ID NO:61 from amino acid 182 to amino acid 248. In further preferred embodiments, the present invention provides a polynucleotide encoding a protein comprising a fragment of the amino acid sequence of SEQ ID NO:61 having biological activity, the fragment preferably comprising eight (more preferably twenty, most preferably thirty) contiguous amino acids of SEQ ID NO:61, or a polynucleotide encoding a protein comprising a fragment of the amino add sequence of SEQ ID NO:61 having biological activity, the fragment comprising the amino acid sequence from amino acid 292 to amino acid 301 of SEQ ID NO:61.

Other embodiments provide the gene corresponding to the cDNA sequence of SEQ ID NO:60.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:60, but excluding the poly(A) tail at the 3′ end of SEQ ID NO:60; and
      (ab) the nucleotide sequence of the cDNA insert of clone CO390_{—}1 deposited with the ATCC under accession number 98232;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:60, but excluding the poly(A) tail at the 3′ end of SEQ ID NO:60; and
      (bb) the nucleotide sequence of the cDNA insert of clone CO390_{—}1 deposited with the ATCC under accession number 98232;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:60, and extending contiguously from a nucleotide sequence corresponding to the 5′ end of SEQ ID NO:60 to a nucleotide sequence corresponding to the 3′ end of SEQ ID NO:60, but excluding the poly(A) tail at the 3′ end of SEQ ID NO:60. Also preferably the polynucleotide isolated according to the above process comprises a nucleotide sequence corresponding to the cDNA sequence of SEQ ID NO:60 from nucleotide 87 to nucleotide 1871, and extending contiguously from a nucleotide sequence corresponding to the 5′ end of said sequence of SEQ ID NO:60 from nucleotide 87 to nucleotide 1871, to a nucleotide sequence corresponding to the 3′ end of said sequence of SEQ ID NO:60 from nucleotide 87 to nucleotide 1871. Also preferably the polynucleotide isolated according to the above process comprises a nucleotide sequence corresponding to the cDNA sequence of SEQ ID NO:60 from nucleotide 628 to nucleotide 1882, and extending contiguously from a nucleotide sequence corresponding to the 5′ end of said sequence of SEQ ID NO:60 from nucleotide 628 to nucleotide 1882, to a nucleotide sequence corresponding to the 3′ end of said sequence of SEQ ID NO:60 from nucleotide 628 to nucleotide 1882.

In other embodiments, the present invention provides a composition comprising a protein, wherein said protein comprises an amino acid sequence selected from the group consisting of:
(a) the amino acid sequence of SEQ ID NO:61;
(b) the amino acid sequence of SEQ ID NO:61 from amino acid 182 to amino acid 248;
(c) a fragment of the amino acid sequence of SEQ ID NO:61, the fragment comprising eight contiguous amino acids of SEQ ID NO:61; and
(d) the amino acid sequence encoded by the cDNA insert of clone CO390_{—}1 deposited with the ATCC under accession number 98232;
   the protein being substantially free from other mammalian proteins. Preferably such protein comprises the amino acid sequence of SEQ ID NO:61 or the amino acid sequence of SEQ ID NO:61 from amino acid 182 to amino acid 248. In further preferred embodiments, the present invention provides a protein comprising a fragment of the amino acid sequence of SEQ ID NO:61 having biological activity, the fragment preferably comprising eight (more preferably twenty, most preferably thirty) contiguous amino acids of SEQ ID NO:61, or a protein comprising a fragment of the amino acid sequence of SEQ ID NO:61 having biological activity, the fragment comprising the amino acid sequence from amino acid 292 to amino acid 301 of SEQ ID NO:61.

In one embodiment, the present invention provides a composition comprising an isolated polynucleotide selected from the group consisting of:
(a) a polynucleotide comprising the nucleotide sequence of SEQ ID NO:62;
(b) a polynucleotide comprising the nucleotide sequence of SEQ ID NO:62 from nucleotide 68 to nucleotide 430;
(c) a polynucleotide comprising the nucleotide sequence of SEQ ID NO:62 from nucleotide 128 to nucleotide 430;
(d) a polynucleotide comprising the nucleotide sequence of the full-length protein coding sequence of clone AJ20_{—}2 deposited with the ATCC under accession number 98261;
(e) a polynucleotide encoding the full-length protein encoded by the cDNA insert of clone AJ20_{—}2 deposited with the ATCC under accession number 98261;
(f) a polynucleotide comprising the nucleotide sequence of a mature protein coding sequence of clone AJ20_{—}2 deposited with the ATCC under accession number 98261;
(g) a polynucleotide encoding a mature protein encoded by the cDNA insert of clone AJ20_{—}2 deposited with the ATCC under accession number 98261;
(h) a polynucleotide encoding a protein comprising the amino acid sequence of SEQ ID NO:63;
(i) a polynucleotide encoding a protein comprising a fragment of the amino acid sequence of SEQ ID NO:63 having biological activity, the fragment comprising eight contiguous amino adds of SEQ ID NO:63;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:62.

Preferably, such polynucleotide comprises the nucleotide sequence of SEQ ID NO:62 from nucleotide 68 to nucleotide 430; the nucleotide sequence of SEQ ID NO:62 from nucleotide 128 to nucleotide 430; the nucleotide sequence of the full-length protein coding sequence of clone AJ20_{—}2 deposited with the ATCC under accession number 98261; or the nucleotide sequence of a mature protein coding sequence of clone AJ20_{—}2 deposited with the ATCC under accession number 98261. In other preferred embodiments, the polynucleotide encodes the full-length or a mature protein encoded by the cDNA insert of clone AJ20_{—}2 deposited with the ATCC under accession number 98261. In further preferred embodiments, the present invention provides a polynucleotide encoding a protein comprising a fragment of the amino acid sequence of SEQ ID NO:63 having biological activity, the fragment preferably comprising eight (more preferably twenty, most preferably thirty) contiguous amino acids of SEQ ID NO:63, or a polynucleotide encoding a protein comprising a fragment of the amino acid sequence of SEQ ID NO:63 having biological activity, the fragment comprising the amino acid sequence from amino acid 55 to amino acid 64 of SEQ ID NO:63.

Other embodiments provide the gene corresponding to the cDNA sequence of SEQ ID NO:62 and SEQ ID NO:64.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:62;
      (ab) SEQ ID NO:64, but excluding the poly(A) tail at the 3′ end of SEQ ID NO:64; and
      (ac) the nucleotide sequence of the cDNA insert of clone AJ20_{—}2 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:62;
      (bb) SEQ ID NO:64, but excluding the poly(A) tail at the 3′ end of SEQ ID NO:64; and
      (bc) the nucleotide sequence of the cDNA insert of clone AJ20_{—}2 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:62 and SEQ ID NO:64, and extending contiguously from a nucleotide sequence corresponding to the 5′ end of SEQ ID NO:62 to a nucleotide sequence corresponding to the 3′ end of SEQ ID NO:64, but excluding the poly(A) tail at the 3′ end of SEQ ID NO:64. Also preferably the polynucleotide isolated according to the above process comprises a nucleotide sequence corresponding to the cDNA sequence of SEQ ID NO:62, and extending contiguously from a nucleotide sequence corresponding to the 5′ end of SEQ ID NO:62 to a nucleotide sequence corresponding to the 3′ end of SEQ ID NO:62. Also preferably the polynucleotide isolated according to the above process comprises a nucleotide sequence corresponding to the cDNA sequence of SEQ ID NO:62 from nucleotide 68 to nucleotide 430, and extending contiguously from a nucleotide sequence corresponding to the 5′ end of said sequence of SEQ ID NO:62 from nucleotide 68 to nucleotide 430, to a nucleotide sequence corresponding to the 3′ end of said sequence of SEQ ID NO:62 from nucleotide 68 to nucleotide 430. Also preferably the polynucleotide isolated according to the above process comprises a nucleotide sequence corresponding to the cDNA sequence of SEQ ID NO:62 from nucleotide 128 to nucleotide 430, and extending contiguously from a nucleotide sequence corresponding to the 5′ end of said sequence of SEQ ID NO:62 from nucleotide 128 to nucleotide 430, to a nucleotide sequence corresponding to the 3′ end of said sequence of SEQ ID NO:62 from nucleotide 128 to nucleotide 430.

In other embodiments, the present invention provides a composition comprising a protein, wherein said protein comprises an amino acid sequence selected from the group consisting of:
(a) the amino acid sequence of SEQ ID NO:63;
(b) a fragment of the amino acid sequence of SEQ ID NO:63, the fragment comprising eight contiguous amino acids of SEQ ID NO:63; and
(c) the amino acid sequence encoded by the cDNA insert of clone AJ20_{—}2 deposited with the ATCC under accession number 98261;
   the protein being substantially free from other mammalian proteins. Preferably such protein comprises the amino acid sequence of SEQ ID NO:63. In further preferred embodiments, the present invention provides a protein comprising a fragment of the amino acid sequence of SEQ ID NO:63 having biological activity, the fragment preferably comprising eight (more preferably twenty, most preferably thirty) contiguous amino acids of SEQ ID NO:63, or a protein comprising a fragment of the amino acid sequence of SEQ ID NO:63 having biological activity, the fragment comprising the amino acid sequence from amino acid 55 to amino acid 64 of SEQ ID NO:63.

In one embodiment, the present invention provides a composition comprising an isolated polynucleotide selected from the group consisting of:
(a) a polynucleotide comprising the nucleotide sequence of SEQ ID NO:66;
(b) a polynucleotide comprising the nucleotide sequence of SEQ ID NO:66 from nucleotide 289 to nucleotide 780;
(c) a polynucleotide comprising the nucleotide sequence of the full-length protein coding sequence of clone AR440_{—}1 deposited with the ATCC under accession number 98261;
(d) a polynucleotide encoding the full-length protein encoded by the cDNA insert of clone AR440_{—}1 deposited with the ATCC under accession number 98261;
(e) a polynucleotide comprising the nucleotide sequence of a mature protein coding sequence of clone AR440_{—}1 deposited with the ATCC under accession number 98261;
(f) a polynucleotide encoding a mature protein encoded by the cDNA insert of clone AR440_{—}1 deposited with the ATCC under accession number 98261;
(g) a polynucleotide encoding a protein comprising the amino acid sequence of SEQ ID NO:67;
(h) a polynucleotide encoding a protein comprising a fragment of the amino acid sequence of SEQ ID NO:67 having biological activity, the fragment comprising eight contiguous amino acids of SEQ ID NO:67;
(i) a polynucleotide which is an allelic variant of a polynucleotide of (a)-(f) above;
a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:66.

Preferably, such polynucleotide comprises the nucleotide sequence of SEQ ID NO:66 from nucleotide 289 to nucleotide 780; the nucleotide sequence of the full-length protein coding sequence of clone AR440_{—}1 deposited with the ATCC under accession number 98261; or the nucleotide sequence of a mature protein coding sequence of clone AR440_{—}1 deposited with the ATCC under accession number 98261. In other preferred embodiments, the polynucleotide encodes the full-length or a mature protein encoded by the cDNA insert of clone AR440_{—}1 deposited with the ATCC under accession number 98261. In yet other preferred embodiments, the present invention provides a polynucleotide encoding a protein comprising the amino acid sequence of SEQ ID NO:67 from amino acid 1 to amino acid 160. In further preferred embodiments, the present invention provides a polynucleotide encoding a protein comprising a fragment of the amino acid sequence of SEQ ID NO:67 having biological activity, the fragment preferably comprising eight (more preferably twenty, most preferably thirty) contiguous amino acids of SEQ ID NO:67, or a polynucleotide encoding a protein comprising a fragment of the amino acid sequence of SEQ ID NO:67 having biological activity, the fragment comprising the amino acid sequence from amino acid 77 to amino acid 86 of SEQ ID NO:67.

Other embodiments provide the gene corresponding to the cDNA sequence of SEQ ID NO:66 and SEQ ID NO:65.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:65;
      (ab) SEQ ID NO:66, but excluding the poly(A) tail at the 3′ end of SEQ ID NO:66; and
      (ac) the nucleotide sequence of the cDNA insert of clone AR440_{—}1 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:65;
      (bb) SEQ ID NO:66, but excluding the poly(A) tail at the 3′ end of SEQ ID NO:66; and
      (bc) the nucleotide sequence of the cDNA insert of clone AR440_{—}1 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:65 and SEQ ID NO:66, and extending contiguously from a nucleotide sequence corresponding to the 5′ end of SEQ ID NO:65 to a nucleotide sequence corresponding to the 3′ end of SEQ ID NO:66, but excluding the poly(A) tail at the 3′ end of SEQ ID NO:66. Also preferably the polynucleotide isolated according to the above process comprises a nucleotide sequence corresponding to the cDNA sequence of SEQ ID NO:66, and extending contiguously from a nucleotide sequence corresponding to the 5′ end of SEQ ID NO:66 to a nucleotide sequence corresponding to the 3′ end of SEQ ID NO:66, but excluding the poly(A) tail at the 3′ end of SEQ ID NO:66. Also preferably the polynucleotide isolated according to the above process comprises a nucleotide sequence corresponding to the cDNA sequence of SEQ ID NO:66 from nucleotide 289 to nucleotide 780, and extending contiguously from a nucleotide sequence corresponding to the 5′ end of said sequence of SEQ ID NO:66 from nucleotide 289 to nucleotide 780, to a nucleotide sequence corresponding to the 3′ end of said sequence of SEQ ID NO:66 from nucleotide 289 to nucleotide 780.

In other embodiments, the present invention provides a composition comprising a protein, wherein said protein comprises an amino acid sequence selected from the group consisting of:
(a) the amino acid sequence of SEQ ID NO:67;
(b) the amino acid sequence of SEQ ID NO:67 from amino acid 1 to amino acid 160;
(c) a fragment of the amino acid sequence of SEQ ID NO:67, the fragment comprising eight contiguous amino acids of SEQ ID NO:67; and
(d) the amino acid sequence encoded by the cDNA insert of clone AR440_{—}1 deposited with the ATCC under accession number 98261;
   the protein being substantially free from other mammalian proteins. Preferably such protein comprises the amino acid sequence of SEQ ID NO:67 or the amino add sequence of SEQ ID NO:67 from amino acid 1 to amino acid 160. In further preferred embodiments, the present invention provides a protein comprising a fragment of the amino acid sequence of SEQ ID NO:67 having biological activity, the fragment preferably comprising eight (more preferably twenty, most preferably thirty) contiguous amino acids of SEQ ID NO:67, or a protein comprising a fragment of the amino acid sequence of SEQ ID NO:67 having biological activity, the fragment comprising the amino acid sequence from amino acid 77 to amino acid 86 of SEQ ID NO:67.

In one embodiment, the present invention provides a composition comprising an isolated polynucleotide selected from the group consisting of:
(a) a polynucleotide comprising the nucleotide sequence of SEQ ID NO:68;
(b) a polynucleotide comprising the nucleotide sequence of SEQ ID NO:68 from nucleotide 76 to nucleotide 1050;
(c) a polynucleotide comprising the nucleotide sequence of SEQ ID NO:68 from nucleotide 331 to nucleotide 567;
(d) a polynucleotide comprising the nucleotide sequence of the full-length protein coding sequence of clone AS164_{—}1 deposited with the ATCC under accession number 98261;
(e) a polynucleotide encoding the full-length protein encoded by the cDNA insert of clone AS164_{—}1 deposited with the ATCC under accession number 98261;
(f) a polynucleotide comprising the nucleotide sequence of a mature protein coding sequence of clone AS164_{—}1 deposited with the ATCC under accession number 98261;
(g) a polynucleotide encoding a mature protein encoded by the cDNA insert of clone AS164_{—}1 deposited with the ATCC under accession number 98261;
(h) a polynucleotide encoding a protein comprising the amino acid sequence of SEQ ID NO:69;
(i) a polynucleotide encoding a protein comprising a fragment of the amino acid sequence of SEQ ID NO:69 having biological activity, the fragment comprising eight contiguous amino acids of SEQ ID NO:69;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:68.

Preferably, such polynucleotide comprises the nucleotide sequence of SEQ ID NO:68 from nucleotide 76 to nucleotide 1050; the nucleotide sequence of SEQ ID NO:68 from nucleotide 331 to nucleotide 567; the nucleotide sequence of the full-length protein coding sequence of clone AS164_{—}1 deposited with the ATCC under accession number 98261; or the nucleotide sequence of a mature protein coding sequence of clone AS164_{—}1 deposited with the ATCC under accession number 98261. In other preferred embodiments, the polynucleotide encodes the full-length or a mature protein encoded by the cDNA insert of clone AS164_{—}1 deposited with the ATCC under accession number 98261. In yet other preferred embodiments, the present invention provides a polynucleotide encoding a protein comprising the amino acid sequence of SEQ ID NO:69 from amino acid 87 to amino acid 164. In further preferred embodiments, the present invention provides a polynucleotide encoding a protein comprising a fragment of the amino acid sequence of SEQ ID NO:69 having biological activity, the fragment preferably comprising eight (more preferably twenty, most preferably thirty) contiguous amino acids of SEQ ID NO:69, or a polynucleotide encoding a protein comprising a fragment of the amino acid sequence of SEQ ID NO:69 having biological activity, the fragment comprising the amino acid sequence from amino acid 157 to amino acid 166 of SEQ ID NO:69. Other embodiments provide the gene corresponding to the cDNA sequence of SEQ ID NO:68.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:68, but excluding the poly(A) tail at the 3′ end of SEQ ID NO:68; and
      (ab) the nucleotide sequence of the cDNA insert of clone AS164_{—}1 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:68, but excluding the poly(A) tail at the 3′ end of SEQ ID NO:68; and
      (bb) the nucleotide sequence of the cDNA insert of clone AS164_{—}1 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:68, and extending contiguously from a nucleotide sequence corresponding to the 5′ end of SEQ ID NO:68 to a nucleotide sequence corresponding to the 3′ end of SEQ ID NO:68, but excluding the poly(A) tail at the 3′ end of SEQ ID NO:68. Also preferably the polynucleotide isolated according to the above process comprises a nucleotide sequence corresponding to the cDNA sequence of SEQ ID NO:68 from nucleotide 76 to nucleotide 1050, and extending contiguously from a nucleotide sequence corresponding to the 5′ end of said sequence of SEQ ID NO:68 from nucleotide 76 to nucleotide 1050, to a nucleotide sequence corresponding to the 3′ end of said sequence of SEQ ID NO:68 from nucleotide 76 to nucleotide 1050. Also preferably the polynucleotide isolated according to the above process comprises a nucleotide sequence corresponding to the cDNA sequence of SEQ ID NO:68 from nucleotide 331 to nucleotide 567, and extending contiguously from a nucleotide sequence corresponding to the 5′ end of said sequence of SEQ ID NO:68 from nucleotide 331 to nucleotide 567, to a nucleotide sequence corresponding to the 3′ end of said sequence of SEQ ID NO:68 from nucleotide 331 to nucleotide 567.

In other embodiments, the present invention provides a composition comprising a protein, wherein said protein comprises an amino acid sequence selected from the group consisting of:
(a) the amino acid sequence of SEQ ID NO:69;
(b) the amino acid sequence of SEQ ID NO:69 from amino acid 87 to amino acid 164;
(c) a fragment of the amino acid sequence of SEQ ID NO:69, the fragment comprising eight contiguous amino acids of SEQ ID NO:69; and
(d) the amino acid sequence encoded by the cDNA insert of clone AS164_{—}1 deposited with the ATCC under accession number 98261;
   the protein being substantially free from other mammalian proteins. Preferably such protein comprises the amino acid sequence of SEQ ID NO:69 or the amino acid sequence of SEQ ID NO:69 from amino acid 87 to amino acid 164. In further preferred embodiments, the present invention provides a protein comprising a fragment of the amino acid sequence of SEQ ID NO:69 having biological activity, the fragment preferably comprising eight (more preferably twenty, most preferably thirty) contiguous amino acids of SEQ ID NO:69, or a protein comprising a fragment of the amino acid sequence of SEQ ID NO:69 having biological activity, the fragment comprising the amino add sequence from amino acid 157 to amino acid 166 of SEQ ID NO:69.

In one embodiment, the present invention provides a composition comprising an isolated polynucleotide selected from the group consisting of:
(a) a polynucleotide comprising the nucleotide sequence of SEQ ID NO:70;
(b) a polynucleotide comprising the nucleotide sequence of SEQ ID NO:70 from nucleotide 242 to nucleotide 1060;
(c) a polynucleotide comprising the nucleotide sequence of SEQ ID NO:70 from nucleotide 596 to nucleotide 1060;
(d) a polynucleotide comprising the nucleotide sequence of SEQ ID NO:70 from nucleotide 10 to nucleotide 373;
(e) a polynucleotide comprising the nucleotide sequence of the full-length protein coding sequence of clone AX8_{—}1 deposited with the ATCC under accession number 98261;
(f) a polynucleotide encoding the full-length protein encoded by the cDNA insert of clone AX8_{—}1 deposited with the ATCC under accession number 98261;
(g) a polynucleotide comprising the nucleotide sequence of a mature protein coding sequence of clone AX8_{—}1 deposited with the ATCC under accession number 98261;
(h) a polynucleotide encoding a mature protein encoded by the cDNA insert of clone AX8_{—}1 deposited with the ATCC under accession number 98261;
(i) a polynucleotide encoding a protein comprising the amino acid sequence of SEQ ID NO:71;
(j) a polynucleotide encoding a protein comprising a fragment of the amino acid sequence of SEQ ID NO:71 having biological activity, the fragment comprising eight contiguous amino acids of SEQ ID NO:71;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:70.

Preferably, such polynucleotide comprises the nucleotide sequence of SEQ ID NO:70 from nucleotide 242 to nucleotide 1060; the nucleotide sequence of SEQ ID NO:70 from nucleotide 596 to nucleotide 1060; the nucleotide sequence of SEQ ID NO:70 from nucleotide 10 to nucleotide 373; the nucleotide sequence of the full-length protein coding sequence of clone AX8_{—}1 deposited with the ATCC under accession number 98261; or the nucleotide sequence of a mature protein coding sequence of clone AX8_{—}1 deposited with the ATCC under accession number 98261. In other preferred embodiments, the polynucleotide encodes the full-length or a mature protein encoded by the cDNA insert of clone AX8_{—}1 deposited with the ATCC under accession number 98261. In yet other preferred embodiments, the present invention provides a polynucleotide encoding a protein comprising the amino acid sequence of SEQ ID NO:71 from amino acid 1 to amino acid 44. In further preferred embodiments, the present invention provides a polynucleotide encoding a protein comprising a fragment of the amino acid sequence of SEQ ID NO:71 having biological activity, the fragment preferably comprising eight (more preferably twenty, most preferably thirty) contiguous amino acids of SEQ ID NO:71, or a polynucleotide encoding a protein comprising a fragment of the amino acid sequence of SEQ ID NO:71 having biological activity, the fragment comprising the amino acid sequence from amino acid 131 to amino acid 140 of SEQ ID NO:71.

Other embodiments provide the gene corresponding to the cDNA sequence of SEQ ID NO:70.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:70, but excluding the poly(A) tail at the 3′ end of SEQ ID NO:70; and
      (ab) the nucleotide sequence of the cDNA insert of clone AX8_{—}1 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:70, but excluding the poly(A) tail at the 3′ end of SEQ ID NO:70; and
      (bb) the nucleotide sequence of the cDNA insert of clone AX8_{—}1 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:70, and extending contiguously from a nucleotide sequence corresponding to the 5′ end of SEQ ID NO:70 to a nucleotide sequence corresponding to the 3′ end of SEQ ID NO:70, but excluding the poly(A) tail at the 3′ end of SEQ ID NO:70. Also preferably the polynucleotide isolated according to the above process comprises a nucleotide sequence corresponding to the cDNA sequence of SEQ ID NO:70 from nucleotide 242 to nucleotide 1060, and extending contiguously from a nucleotide sequence corresponding to the 5′ end of said sequence of SEQ ID NO:70 from nucleotide 242 to nucleotide 1060, to a nucleotide sequence corresponding to the 3′ end of said sequence of SEQ ID NO:70 from nucleotide 242 to nucleotide 1060. Also preferably the polynucleotide isolated according to the above process comprises a nucleotide sequence corresponding to the cDNA sequence of SEQ ID NO:70 from nucleotide 596 to nucleotide 1060, and extending contiguously from a nucleotide sequence corresponding to the 5′ end of said sequence of SEQ ID NO:70 from nucleotide 596 to nucleotide 1060, to a nucleotide sequence corresponding to the ˜3′ end of said sequence of SEQ ID NO:70 from nucleotide 596 to nucleotide 1060. Also preferably the polynucleotide isolated according to the above process comprises a nucleotide sequence corresponding to the cDNA sequence of SEQ ID NO:70 from nucleotide 10 to nucleotide 373, and extending contiguously from a nucleotide sequence corresponding to the 5′ end of said sequence of SEQ ID NO:70 from nucleotide 10 to nucleotide 373, to a nucleotide sequence corresponding to the 3′ end of said sequence of SEQ ID NO:70 from nucleotide 10 to nucleotide 373.

In other embodiments, the present invention provides a composition comprising a protein, wherein said protein comprises an amino acid sequence selected from the group consisting of:
(a) the amino acid sequence of SEQ ID NO:71;
(b) the amino acid sequence of SEQ ID NO:71 from amino acid 1 to amino acid 44;
(c) a fragment of the amino acid sequence of SEQ ID NO:71, the fragment comprising eight contiguous amino acids of SEQ ID NO:71; and
(d) the amino acid sequence encoded by the cDNA insert of clone AX8_{—}1 deposited with the ATCC under accession number 98261;
the protein being substantially free from other mammalian proteins. Preferably such protein comprises the amino acid sequence of SEQ ID NO:71 or the amino acid sequence of SEQ ID NO:71 from amino acid 1 to amino acid 44. In further preferred embodiments, the present invention provides a protein comprising a fragment of the amino acid sequence of SEQ ID NO:71 having biological activity, the fragment preferably comprising eight (more preferably twenty, most preferably thirty) contiguous amino acids of SEQ ID NO:71, or a protein comprising a fragment of the amino acid sequence of SEQ ID NO:71 having biological activity, the fragment comprising the amino acid sequence from amino acid 131 to amino acid 140 of SEQ ID NO:71.

In one embodiment, the present invention provides a composition comprising an isolated polynucleotide selected from the group consisting of:
(a) a polynucleotide comprising the nucleotide sequence of SEQ ID NO:72;
(b) a polynucleotide comprising the nucleotide sequence of SEQ ID NO:72 from nucleotide 773 to nucleotide 928;
(c) a polynucleotide comprising the nucleotide sequence of SEQ ID NO:72 from nucleotide 815 to nucleotide 928;
(d) a polynucleotide comprising the nucleotide sequence of the full-length protein coding sequence of clone BD176_{—}3 deposited with the ATCC under accession number 98261;
(e) a polynucleotide encoding the full-length protein encoded by the cDNA insert of clone BD176_{—}3 deposited with the ATCC under accession number 98261;
(f) a polynucleotide comprising the nucleotide sequence of a mature protein coding sequence of clone BD176_{—}3 deposited with the ATCC under accession number 98261;
(g) a polynucleotide encoding a mature protein encoded by the cDNA insert of clone BD176_{—}3 deposited with the ATCC under accession number 98261;
(h) a polynucleotide encoding a protein comprising the amino acid sequence of SEQ ID NO:73;
(i) a polynucleotide encoding a protein comprising a fragment of the amino acid sequence of SEQ ID NO:73 having biological activity, the fragment comprising eight contiguous amino acids of SEQ ID NO:73;
(j) a polynucleotide which is an allelic variant of a polynucleotide of
(a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a>(i) and that has a length that is at least 25% of the length of SEQ ID NO:72.

Preferably, such polynucleotide comprises the nucleotide sequence of SEQ ID NO:72 from nucleotide 773 to nucleotide 928; the nucleotide sequence of SEQ ID NO:72 from nucleotide 815 to nucleotide 928; the nucleotide sequence of the full-length protein coding sequence of clone BD176_{—}3 deposited with the ATCC under accession number 98261; or the nucleotide sequence of a mature protein coding sequence of clone BD176_{—}3 deposited with the ATCC under accession number 98261. In other preferred embodiments, the polynucleotide encodes the full-length or a mature protein encoded by the cDNA insert of clone BD176_{—}3 deposited with the ATCC under accession number 98261. In further preferred embodiments, the present invention provides a polynucleotide encoding a protein comprising a fragment of the amino acid sequence of SEQ ID NO:73 having biological activity, the fragment preferably comprising eight (more preferably twenty, most preferably thirty) contiguous amino acids of SEQ ID NO:73, or a polynucleotide encoding a protein comprising a fragment of the amino acid sequence of SEQ ID NO:73 having biological activity, the fragment comprising the amino acid sequence from amino acid 21 to amino acid 30 of SEQ ID NO:73.

Other embodiments provide the gene corresponding to the cDNA sequence of SEQ ID NO:72 and SEQ ID NO:74.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:72;
      (ab) SEQ ID NO:74, but excluding the poly(A) tail at the 3′ end of SEQ ID NO:74; and
      (ac) the nucleotide sequence of the cDNA insert of clone BD176_{—}3 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:72;
      (bb) SEQ ID NO:74, but excluding the poly(A) tail at the 3′ end of SEQ ID NO:74; and
      (bc) the nucleotide sequence of the cDNA insert of clone BD176_{—}3 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:72 and SEQ ID NO:74, and extending contiguously from a nucleotide sequence corresponding to the 5′ end of SEQ ID NO:72 to a nucleotide sequence corresponding to the 3′ end of SEQ ID NO:74, but excluding the poly(A) tail at the 3′ end of SEQ ID NO:74. Also preferably the polynucleotide isolated according to the above process comprises a nucleotide sequence corresponding to the cDNA sequence of SEQ ID NO:72, and extending contiguously from a nucleotide sequence corresponding to the 5′ end of SEQ ID NO:72 to a nucleotide sequence corresponding to the 3′ end of SEQ ID NO:72. Also preferably the polynucleotide isolated according to the above process comprises a nucleotide sequence corresponding to the cDNA sequence of SEQ ID NO:72 from nucleotide 773 to nucleotide 928, and extending contiguously from a nucleotide sequence corresponding to the 5′ end of said sequence of SEQ ID NO:72 from nucleotide 773 to nucleotide 928, to a nucleotide sequence corresponding to the 3′ end of said sequence of SEQ ID NO:72 from nucleotide 773 to nucleotide 928. Also preferably the polynucleotide isolated according to the above process comprises a nucleotide sequence corresponding to the cDNA sequence of SEQ ID NO:72 from nucleotide 815 to nucleotide 928, and extending contiguously from a nucleotide sequence corresponding to the 5′ end of said sequence of SEQ ID NO:72 from nucleotide 815 to nucleotide 928, to a nucleotide sequence corresponding to the 3′ end of said sequence of SEQ ID NO:72 from nucleotide 815 to nucleotide 928.

In other embodiments, the present invention provides a composition comprising a protein, wherein said protein comprises an amino acid sequence selected from the group consisting of:
(a) the amino acid sequence of SEQ ID NO:73;
(b) a fragment of the amino acid sequence of SEQ ID NO:73, the fragment comprising eight contiguous amino acids of SEQ ID NO:73; and
(c) the amino acid sequence encoded by the cDNA insert of clone BD176_{—}3 deposited with the ATCC under accession number 98261;
   the protein being substantially free from other mammalian proteins. Preferably such protein comprises the amino acid sequence of SEQ ID NO:73. In further preferred embodiments, the present invention provides a protein comprising a fragment of the amino add sequence of SEQ ID NO:73 having biological activity, the fragment preferably comprising eight (more preferably twenty, most preferably thirty) contiguous amino acids of SEQ ID NO:73, or a protein comprising a fragment of the amino acid sequence of SEQ ID NO:73 having biological activity, the fragment comprising the amino acid sequence from amino acid 21 to amino acid 30 of SEQ ID NO:73.

In one embodiment, the present invention provides a composition comprising an isolated polynucleotide selected from the group consisting of:
(a) a polynucleotide comprising the nucleotide sequence of SEQ ID NO:75;
(b) a polynucleotide comprising the nucleotide sequence of SEQ ID NO:75 from nucleotide 174 to nucleotide 440;
(c) a polynucleotide comprising the nucleotide sequence of SEQ ID NO:75 from nucleotide 1 to nucleotide 313;
(d) a polynucleotide comprising the nucleotide sequence of the full-length protein coding sequence of clone BD339_{—}1 deposited with the ATCC under accession number 98261;
(e) a polynucleotide encoding the full-length protein encoded by the cDNA insert of clone BD339_{—}1 deposited with the ATCC under accession number 98261;
(f) a polynucleotide comprising the nucleotide sequence of a mature protein coding sequence of clone BD339_{—}1 deposited with the ATCC under accession number 98261;
(g) a polynucleotide encoding a mature protein encoded by the cDNA insert of clone BD339_{—}1 deposited with the ATCC under accession number 98261;
(h) a polynucleotide encoding a protein comprising the amino acid sequence of SEQ ID NO:76;
(i) a polynucleotide encoding a protein comprising a fragment of the amino acid sequence of SEQ ID NO:76 having biological activity, the fragment comprising eight contiguous amino acids of SEQ ID NO:76;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a>(i) and that has a length that is at least 25% of the length of SEQ ID NO:75.

Preferably, such polynucleotide comprises the nucleotide sequence of SEQ ID NO:75 from nucleotide 174 to nucleotide 440; the nucleotide sequence of SEQ ID NO:75 from nucleotide 1 to nucleotide 313; the nucleotide sequence of the full-length protein coding sequence of clone BD339_{—}1 deposited with the ATCC under accession number 98261; or the nucleotide sequence of a mature protein coding sequence of clone BD339_{—}1 deposited with the ATCC under accession number 98261. In other preferred embodiments, the polynucleotide encodes the full-length or a mature protein encoded by the cDNA insert of clone BD339_{—}1 deposited with the. ATCC under accession number 98261. In yet other preferred embodiments, the present invention provides a polynucleotide encoding a protein comprising the amino acid sequence of SEQ ID NO:76 from amino acid 1 to amino add 46. In further preferred embodiments, the present invention provides a polynucleotide encoding a protein comprising a fragment of the amino acid sequence of SEQ ID NO:76 having biological activity, the fragment preferably comprising eight (more preferably twenty, most preferably thirty) contiguous amino acids of SEQ ID NO:76, or a polynucleotide encoding a protein comprising a fragment of the amino acid sequence of SEQ ID NO:76 having biological activity, the fragment comprising the amino acid sequence from amino acid 39 to amino acid 48 of SEQ ID NO:76.

Other embodiments provide the gene corresponding to the cDNA sequence of SEQ ID NO:75.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:75, but excluding the poly(A) tail at the 3′ end of SEQ ID NO:75; and
      (ab) the nucleotide sequence of the cDNA insert of clone BD339_{—}1 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:75, but excluding the poly(A) tail at the 3′ end of SEQ ID NO:75; and
      (bb) the nucleotide sequence of the cDNA insert of clone BD339_{—}1 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:75, and extending contiguously from a nucleotide sequence corresponding to the 5′ end of SEQ ID NO:75 to a nucleotide sequence corresponding to the 3′ end of SEQ ID NO:75, but excluding the poly(A) tail at the 3′ end of SEQ ID NO:75. Also preferably the polynucleotide isolated according to the above process comprises a nucleotide sequence corresponding to the cDNA sequence of SEQ ID NO:75 from nucleotide 174 to nucleotide 440, and extending contiguously from a nucleotide sequence corresponding to the 5′ end of said sequence of SEQ ID NO:75 from nucleotide 174 to nucleotide 440, to a nucleotide sequence corresponding to the 3′ end of said sequence of SEQ ID NO:75 from nucleotide 174 to nucleotide 440. Also preferably the polynucleotide isolated according to the above process comprises a nucleotide sequence corresponding to the cDNA sequence of SEQ ID NO:75 from nucleotide 1 to nucleotide 313, and extending contiguously from a nucleotide sequence corresponding to the 5′ end of said sequence of SEQ ID NO:75 from nucleotide 1 to nucleotide 313, to a nucleotide sequence corresponding to the 3′ end of said sequence of SEQ ID NO:75 from nucleotide 1 to nucleotide 313.

In other embodiments, the present invention provides a composition comprising a protein, wherein said protein comprises an amino acid sequence selected from the group consisting of:
(a) the amino acid sequence of SEQ ID NO:76;
(b) the amino acid sequence of SEQ ID NO:76 from amino acid 1 to amino acid 46;
(c) a fragment of the amino acid sequence of SEQ ID NO:76, the fragment comprising eight contiguous amino acids of SEQ ID NO:76; and
(d) the amino acid sequence encoded by the cDNA insert of clone BD339_{—}1 deposited with the ATCC under accession number 98261;
   the protein being substantially free from other mammalian proteins. Preferably such protein comprises the amino acid sequence of SEQ ID NO:76 or the amino acid sequence of SEQ ID NO:76 from amino acid 1 to amino acid 46. In further preferred embodiments, the present invention provides a protein comprising a fragment of the amino acid sequence of SEQ ID NO:76 having biological activity, the fragment preferably comprising eight (more preferably twenty, most preferably thirty) contiguous amino acids of SEQ ID NO:76, or a protein comprising a fragment of the amino acid sequence of SEQ ID NO:76 having biological activity, the fragment comprising the amino acid sequence from amino acid 39 to amino acid 48 of SEQ ID NO:76.

In one embodiment, the present invention provides a composition comprising an isolated polynucleotide selected from the group consisting of:
(a) a polynucleotide comprising the nucleotide sequence of SEQ ID NO:77;
(b) a polynucleotide comprising the nucleotide sequence of SEQ ID NO:77 from nucleotide 509 to nucleotide 619;
(c) a polynucleotide comprising the nucleotide sequence of SEQ ID NO:77 from nucleotide 1 to nucleotide 580;
(d) a polynucleotide comprising the nucleotide sequence of the full-length protein coding sequence of clone BD427_{—}1 deposited with the ATCC under accession number 98261;
(e) a polynucleotide encoding the full-length protein encoded by the cDNA insert of clone BD427_{—}1 deposited with the ATCC under accession number 98261;
(f) a polynucleotide comprising the nucleotide sequence of a mature protein coding sequence of clone BD427_{—}1 deposited with the ATCC under accession number 98261;
(g) a polynucleotide encoding a mature protein encoded by the cDNA insert of clone BD427_{—}1 deposited with the ATCC under accession number 98261;
(h) a polynucleotide encoding a protein comprising the amino acid sequence of SEQ ID NO:78;
(i) a polynucleotide encoding a protein comprising a fragment of the amino acid sequence of SEQ ID NO:78 having biological activity, the fragment comprising eight contiguous amino acids of SEQ ID NO:78;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:77.

Preferably, such polynucleotide comprises the nucleotide sequence of SEQ ID NO:77 from nucleotide 509 to nucleotide 619; the nucleotide sequence of SEQ ID NO:77 from nucleotide 1 to nucleotide 580; the nucleotide sequence of the full-length protein coding sequence of clone BD427_{—}1 deposited with the ATCC under accession number 98261; or the nucleotide sequence of a mature protein coding sequence of clone BD427_{—}1 deposited with the ATCC under accession number 98261. In other preferred embodiments, the polynucleotide encodes the full-length or a mature protein encoded by the cDNA insert of clone BD427_{—}1 deposited with the ATCC under accession number 98261. In yet other preferred embodiments, the present invention provides a polynucleotide encoding a protein comprising the amino acid sequence of SEQ ID NO:78 from amino acid 1 to amino acid 24. In further preferred embodiments, the present invention provides a polynucleotide encoding a protein comprising a fragment of the amino acid sequence of SEQ ID NO:78 having biological activity, the fragment preferably comprising eight (more preferably twenty, most preferably thirty) contiguous amino acids of SEQ ID NO:78, or a polynucleotide encoding a protein comprising a fragment of the amino acid sequence of SEQ ID NO:78 having biological activity, the fragment comprising the amino acid sequence from amino acid 13 to amino acid 22 of SEQ ID NO:78.

Other embodiments provide the gene corresponding to the cDNA sequence of SEQ ID NO:77.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:77, but excluding the poly(A) tail at the 3′ end of SEQ ID NO:77; and
      (ab) the nucleotide sequence of the cDNA insert of clone BD427_{—}1 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:77, but excluding the poly(A) tail at the 3′ end of SEQ ID NO:77; and
      (bb) the nucleotide sequence of the cDNA insert of clone BD427_{—}1 deposited with the ATCC under accession number 98261;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:77, and extending contiguously from a nucleotide sequence corresponding to the 5′ end of SEQ ID NO:77 to a nucleotide sequence corresponding to the 3′ end of SEQ ID NO:77, but excluding the poly(A) tail at the 3′ end of SEQ ID NO:77. Also preferably the polynucleotide isolated according to the above process comprises a nucleotide sequence corresponding to the cDNA sequence of SEQ ID NO:77 from nucleotide 509 to nucleotide 619, and extending contiguously from a nucleotide sequence corresponding to the 5′ end of said sequence of SEQ ID NO:77 from nucleotide 509 to nucleotide 619, to a nucleotide sequence corresponding to the 3′ end of said sequence of SEQ ID NO:77 from nucleotide 509 to nucleotide 619. Also preferably the polynucleotide isolated according to the above process comprises a nucleotide sequence corresponding to the cDNA sequence of SEQ ID NO:77 from nucleotide 1 to nucleotide 580, and extending contiguously from a nucleotide sequence corresponding to the 5′ end of said sequence of SEQ ID NO:77 from nucleotide 1 to nucleotide 580, to a nucleotide sequence corresponding to the 3′ end of said sequence of SEQ ID NO:77 from nucleotide 1 to nucleotide 580.

In other embodiments, the present invention provides a composition comprising a protein, wherein said protein comprises an amino acid sequence selected from the group consisting of:
(a) the amino acid sequence of SEQ ID NO:78;
(b) the amino acid sequence of SEQ ID NO:78 from amino acid 1 to amino acid 24;
(c) a fragment of the amino acid sequence of SEQ ID NO:78, the fragment comprising eight contiguous amino adds of SEQ ID NO:78; and
(d) the amino acid sequence encoded by the cDNA insert of clone BD427_{—}1 deposited with the ATCC under accession number 98261;
   the protein being substantially free from other mammalian proteins. Preferably such protein comprises the amino acid sequence of SEQ ID NO:78 or the amino acid sequence of SEQ ID NO:78 from amino acid 1 to amino acid 24. In further preferred embodiments, the present invention provides a protein comprising a fragment of the amino acid sequence of SEQ ID NO:78 having biological activity, the fragment preferably comprising eight (more preferably twenty, most preferably thirty) contiguous amino acids of SEQ ID NO:78, or a protein comprising a fragment of the amino acid sequence of SEQ ID NO:78 having biological activity, the fragment comprising the amino acid sequence from amino acid 13 to amino acid 22 of SEQ ID NO:78.

In one embodiment, the present invention provides a composition comprising an isolated polynucleotide selected from the group consisting of:
(a) a polynucleotide comprising the nucleotide sequence of SEQ ID NO:79;
(b) a polynucleotide comprising the nucleotide sequence of SEQ ID NO:79 from nucleotide 300 to nucleotide 360;
(c) a polynucleotide comprising the nucleotide sequence of the full-length protein coding sequence of clone BL229_{—}22 deposited with the ATCC under accession number 98261;
(d) a polynucleotide encoding the full-length protein encoded by the cDNA insert of clone BL229_{—}22 deposited with the ATCC under accession number 98261;
(e) a polynucleotide comprising the nucleotide sequence of a mature protein coding sequence of clone BL229_{—}22 deposited with the ATCC under accession number 98261;
(f) a polynucleotide encoding a mature protein encoded by the cDNA insert of clone BL229_{—}22 deposited with the ATCC under accession number 98261;
(g) a polynucleotide encoding a protein comprising the amino acid sequence of SEQ ID NO:80;
(h) a polynucleotide encoding a protein comprising a fragment of the amino acid sequence of SEQ ID NO:80 having biological activity, the fragment comprising eight contiguous amino acids of SEQ ID NO:80;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:79.

Preferably, such polynucleotide comprises the nucleotide sequence of SEQ ID NO:79 from nucleotide 300 to nucleotide 360; the nucleotide sequence of the full-length protein coding sequence of clone BL229_{—}22 deposited with the ATCC under accession number 98261; or the nucleotide sequence of a mature protein coding sequence of clone BL229_{—}22 deposited with the ATCC under accession number 98261. In other preferred embodiments, the polynucleotide encodes the full-length or a mature protein encoded by the cDNA insert of clone BL229_{—}22 deposited with the ATCC under accession number 98261. In further preferred embodiments, the present invention provides a polynucleotide encoding a protein comprising a fragment of the amino acid sequence of SEQ ID NO:80 having biological activity, the fragment preferably comprising eight (more preferably twenty, most preferably thirty) contiguous amino acids of SEQ ID NO:80, or a polynucleotide encoding a protein comprising a fragment of the amino acid sequence of SEQ ID NO:80 having biological activity, the fragment comprising the amino acid sequence from amino acid 5 to amino acid 14 of SEQ ID NO:80.

Other embodiments provide the gene corresponding to the cDNA sequence of SEQ ID NO:79 and SEQ ID NO:81.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:79;
      (ab) SEQ ID NO:81, but excluding the poly(A) tail at the 3′ end of SEQ ID NO:81; and
      (ac) the nucleotide sequence of the cDNA insert of clone BL229_{—}22 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:79;
      (bb) SEQ ID NO:81, but excluding the poly(A) tail at the 3′ end of SEQ ID NO:81; and
      (bc) the nucleotide sequence of the cDNA insert of clone BL229_{—}22 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:79 and SEQ ID NO:81, and extending contiguously from a nucleotide sequence corresponding to the 5′ end of SEQ ID NO:79 to a nucleotide sequence corresponding to the 3′ end of SEQ ID NO:81, but excluding the poly(A) tail at the 3′ end of SEQ ID NO:81. Also preferably the polynucleotide isolated according to the above process comprises a nucleotide sequence corresponding to the cDNA sequence of SEQ ID NO:79, and extending contiguously from a nucleotide sequence corresponding to the 5′ end of SEQ ID NO:79 to a nucleotide sequence corresponding to the 3′ end of SEQ ID NO:79. Also preferably the polynucleotide isolated according to the above process comprises a nucleotide sequence corresponding to the cDNA sequence of SEQ ID NO:79 from nucleotide 300 to nucleotide 360, and extending contiguously from a nucleotide sequence corresponding to the 5′ end of said sequence of SEQ ID NO:79 from nucleotide 300 to nucleotide 360, to a nucleotide sequence corresponding to the 3′ end of said sequence of SEQ ID NO:79 from nucleotide 300 to nucleotide 360.

In other embodiments, the present invention provides a composition comprising a protein, wherein said protein comprises an amino acid sequence selected from the group consisting of:
(a) the amino acid sequence of SEQ ID NO:80;
(b) a fragment of the amino acid sequence of SEQ ID NO:80, the fragment comprising eight contiguous amino acids of SEQ ID NO:80; and
(c) the amino add sequence encoded by the cDNA insert of clone BL229_{—}22 deposited with the ATCC under accession number 98261;
   the protein being substantially free from other mammalian proteins. Preferably such protein comprises the amino acid sequence of SEQ ID NO:80. In further preferred embodiments, the present invention provides a protein comprising a fragment of the amino acid sequence of SEQ ID NO:80 having biological activity, the fragment preferably comprising eight (more preferably twenty, most preferably thirty) contiguous amino acids of SEQ ID NO:80, or a protein comprising a fragment of the amino acid sequence of SEQ ID NO:80 having biological activity, the fragment comprising the amino acid sequence from amino acid 5 to amino acid 14 of SEQ ID NO:80.

In one embodiment, the present invention provides a composition comprising an isolated polynucleotide selected from the group consisting of:
(a) a polynucleotide comprising the nucleotide sequence of SEQ ID NO:82;
(b) a polynucleotide comprising the nucleotide sequence of SEQ ID NO:82 from nucleotide 604 to nucleotide 771;
(c) a polynucleotide comprising the nucleotide sequence of SEQ ID NO:82 from nucleotide 1 to nucleotide 684;
(d) a polynucleotide comprising the nucleotide sequence of the full-length protein coding sequence of clone BV123_{—}16 deposited with the ATCC under accession number 98261;
(e) a polynucleotide encoding the full-length protein encoded by the cDNA insert of clone BV123_{—}16 deposited with the ATCC under accession number 98261;
(f) a polynucleotide comprising the nucleotide sequence of a mature protein coding sequence of clone BV123_{—}16 deposited with the ATCC under accession number 98261;
(g) a polynucleotide encoding a mature protein encoded by the cDNA insert of clone BV123_{—}16 deposited with the ATCC under accession number 98261;
(h) a polynucleotide encoding a protein comprising the amino acid sequence of SEQ ID NO:83;
(i) a polynucleotide encoding a protein comprising a fragment of the amino acid sequence of SEQ ID NO:83 having biological activity, the fragment comprising eight contiguous amino acids of SEQ ID NO:83;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:82.

Preferably, such polynucleotide comprises the nucleotide sequence of SEQ ID NO:82 from nucleotide 604 to nucleotide 771; the nucleotide sequence of SEQ ID NO:82 from nucleotide 1 to nucleotide 684; the nucleotide sequence of the full-length protein coding sequence of clone BV123_{—}16 deposited with the ATCC under accession number 98261; or the nucleotide sequence of a mature protein coding sequence of clone BV123_{—}16 deposited with the ATCC under accession number 98261. In other preferred embodiments, the polynucleotide encodes the full-length or a mature protein encoded by the cDNA insert of clone BV123_{—}16 deposited with the ATCC under accession number 98261. In yet other preferred embodiments, the present invention provides a polynucleotide encoding a protein comprising the amino acid sequence of SEQ ID NO:83 from amino acid 1 to amino acid 27. In further preferred embodiments, the present invention provides a polynucleotide encoding a protein comprising a fragment of the amino acid sequence of SEQ ID NO:83 having biological activity, the fragment preferably comprising eight (more preferably twenty, most preferably thirty) contiguous amino acids of SEQ ID NO:83, or a polynucleotide encoding a protein comprising a fragment of the amino acid sequence of SEQ ID NO:83 having biological activity, the fragment comprising the amino acid sequence from amino acid 23 to amino acid 32 of SEQ ID NO:83.

Other embodiments provide the gene corresponding to the cDNA sequence of SEQ ID NO:82.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:82, but excluding the poly(A) tail at the 3′ end of SEQ ID NO:82; and
      (ab) the nucleotide sequence of the cDNA of clone BV123_{—}16 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:82, but excluding the poly(A) tail at the 3′ end of SEQ ID NO:82; and
      (bb) the nucleotide sequence of the cDNA insert of clone BV123_{—}16 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:82, and extending contiguously from a nucleotide sequence corresponding to the 5′ end of SEQ ID NO:82 to a nucleotide sequence corresponding to the 3′ end of SEQ ID NO:82, but excluding the poly(A) tail at the 3′ end of SEQ ID NO:82. Also preferably the polynucleotide isolated according to the above process comprises a nucleotide sequence corresponding to the cDNA sequence of SEQ ID NO:82 from nucleotide 604 to nucleotide 771, and extending contiguously from a nucleotide sequence corresponding to the 5′ end of said sequence of SEQ ID NO:82 from nucleotide 604 to nucleotide 771, to a nucleotide sequence corresponding to the 3′ end of said sequence of SEQ ID NO:82 from nucleotide 604 to nucleotide 771. Also preferably the polynucleotide isolated according to the above process comprises a nucleotide sequence corresponding to the cDNA sequence of SEQ ID NO:82 from nucleotide 1 to nucleotide 684, and extending contiguously from a nucleotide sequence corresponding to the 5′ end of said sequence of SEQ ID NO:82 from nucleotide 1 to nucleotide 684, to a nucleotide sequence corresponding to the 3′ end of said sequence of SEQ ID NO:82 from nucleotide 1 to nucleotide 684.

In other embodiments, the present invention provides a composition comprising a protein, wherein said protein comprises an amino acid sequence selected from the group consisting of:
(a) the amino acid sequence of SEQ ID NO:83;
(b) the amino acid sequence of SEQ ID NO:83 from amino acid 1 to amino acid 27;
(c) a fragment of the amino acid sequence of SEQ ID NO:83, the fragment comprising eight contiguous amino acids of SEQ ID NO:83; and
(d) the amino acid sequence encoded by the cDNA insert of clone BV123_{—}16 deposited with the ATCC under accession number 98261;
   the protein being substantially free from other mammalian proteins. Preferably such protein comprises the amino acid sequence of SEQ ID NO:83 or the amino acid sequence of SEQ ID NO:83 from amino acid 1 to amino acid 27. In further preferred embodiments, the present invention provides a protein comprising a fragment of the amino acid sequence of SEQ ID NO:83 having biological activity, the fragment preferably comprising eight (more preferably twenty, most preferably thirty) contiguous amino acids of SEQ ID NO:83, or a protein comprising a fragment of the amino acid sequence of SEQ ID NO:83 having biological activity, the fragment comprising the amino acid sequence from amino acid 23 to amino acid 32 of SEQ ID NO:83.

In one embodiment, the present invention provides a composition comprising an isolated polynucleotide selected from the group consisting of:
(a) a polynucleotide comprising the nucleotide sequence of SEQ ID NO:84;
(b) a polynucleotide comprising the nucleotide sequence of SEQ ID NO:84 from nucleotide 43 to nucleotide 297;
(c) a polynucleotide comprising the nucleotide sequence of SEQ ID NO:84 from nucleotide 94 to nucleotide 297;
(d) a polynucleotide comprising the nucleotide sequence of SEQ ID NO:84 from nucleotide 1 to nucleotide 379;
(e) a polynucleotide comprising the nucleotide sequence of the full-length protein coding sequence of clone CH377_{—}1 deposited with the ATCC under accession number 98261;
(f) a polynucleotide encoding the full-length protein encoded by the cDNA insert of clone CH377_{—}1 deposited with the ATCC under accession number 98261;
(g) a polynucleotide comprising the nucleotide sequence of a mature protein coding sequence of clone CH377_{—}1 deposited with the ATCC under accession number 98261;
(h) a polynucleotide encoding a mature protein encoded by the cDNA insert of clone CH377_{—}1 deposited with the ATCC under accession number 98261;
(i) a polynucleotide encoding a protein comprising the amino acid sequence of SEQ ID NO:85;
(j) a polynucleotide encoding a protein comprising a fragment of the amino acid sequence of SEQ ID NO:85 having biological activity, the fragment comprising eight contiguous amino acids of SEQ ID NO:85;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:84.

Preferably, such polynucleotide comprises the nucleotide sequence of SEQ ID NO:84 from nucleotide 43 to nucleotide 297; the nucleotide sequence of SEQ ID NO:84 from nucleotide 94 to nucleotide 297; the nucleotide sequence of SEQ ID NO:84 from nucleotide 1 to nucleotide 379; the nucleotide sequence of the full-length protein coding sequence of clone CH377_{—}1 deposited with the ATCC under accession number 98261; or the nucleotide sequence of a mature protein coding sequence of clone CH377_{—}1 deposited with the ATCC under accession number 98261. In other preferred embodiments, the polynucleotide encodes the full-length or a mature protein encoded by the cDNA insert of clone CH377_{—}1 deposited with the ATCC under accession number 98261. In further preferred embodiments, the present invention provides a polynucleotide encoding a protein comprising a fragment of the amino acid sequence of SEQ ID NO:85 having biological activity, the fragment preferably comprising eight (more preferably twenty, most preferably thirty) contiguous amino acids of SEQ ID NO:85, or a polynucleotide encoding a protein comprising a fragment of the amino acid sequence of SEQ ID NO:85 having biological activity, the fragment comprising the amino acid sequence from amino acid 37 to amino acid 46 of SEQ ID NO:85.

Other embodiments provide the gene corresponding to the cDNA sequence of SEQ ID NO:84.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:84, but excluding the poly(A) tail at the 3′ end of SEQ ID NO:84; and
      (ab) the nucleotide sequence of the cDNA insert of clone CH377_{—}1 deposited with the ATCC under accession number 98261;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:84, but excluding the poly(A) tail at the 3′ end of SEQ ID NO:84; and
      (bb) the nucleotide sequence of the cDNA insert of clone CH377_{—}1 deposited with the ATCC under accession number 98261;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:84, and extending contiguously from a nucleotide sequence corresponding to the 5′ end of SEQ ID NO:84 to a nucleotide sequence corresponding to the 3′ end of SEQ ID NO:84, but excluding the poly(A) tail at the 3′ end of SEQ ID NO:84. Also preferably the polynucleotide isolated according to the above process comprises a nucleotide sequence corresponding to the cDNA sequence of SEQ ID NO:84 from nucleotide 43 to nucleotide 297, and extending contiguously from a nucleotide sequence corresponding to the 5′ end of said sequence of SEQ ID NO:84 from nucleotide 43 to nucleotide 297, to a nucleotide sequence corresponding to the 3′ end of said sequence of SEQ ID NO:84 from nucleotide 43 to nucleotide 297. Also preferably the polynucleotide isolated according to the above process comprises a nucleotide sequence corresponding to the cDNA sequence of SEQ ID NO:84 from nucleotide 94 to nucleotide 297, and extending contiguously from a nucleotide sequence corresponding to the 5′ end of said sequence of SEQ ID NO:84 from nucleotide 94 to nucleotide 297, to a nucleotide sequence corresponding to the 3′ end of said sequence of SEQ ID NO:84 from nucleotide 94 to nucleotide 297. Also preferably the polynucleotide isolated according to the above process comprises a nucleotide sequence corresponding to the cDNA sequence of SEQ ID NO:84 from nucleotide 1 to nucleotide 379, and extending contiguously from a nucleotide sequence corresponding to the 5′ end of said sequence of SEQ ID NO:84 from nucleotide 1 to nucleotide 379, to a nucleotide sequence corresponding to the 3′ end of said sequence of SEQ ID NO:84 from nucleotide 1 to nucleotide 379.

In other embodiments, the present invention provides a composition comprising a protein, wherein said protein comprises an amino acid sequence selected from the group consisting of:
(a) the amino acid sequence of SEQ ID NO:85;
(b) a fragment of the amino acid sequence of SEQ ID NO:85, the fragment comprising eight contiguous amino acids of SEQ ID NO:85; and
(c) the amino acid sequence encoded by the cDNA insert of clone CH377_{—}1 deposited with the ATCC under accession number 98261;
   the protein being substantially free from other mammalian proteins. Preferably such protein comprises the amino add sequence of SEQ ID NO:85. In further preferred embodiments, the present invention provides a protein comprising a fragment of the amino acid sequence of SEQ ID NO:85 having biological activity, the fragment preferably comprising eight (more preferably twenty, most preferably thirty) contiguous amino adds of SEQ ID NO:85, or a protein comprising a fragment of the amino acid sequence of SEQ ID NO:85 having biological activity, the fragment comprising the amino acid sequence from amino acid 37 to amino acid 46 of SEQ ID NO:85.

In one embodiment, the present invention provides a composition comprising an isolated polynucleotide selected from the group consisting of:
(a) a polynucleotide comprising the nucleotide sequence of SEQ ID NO:87;
(b) a polynucleotide comprising the nucleotide sequence of SEQ ID NO:87 from nucleotide 390 to nucleotide 563;
(c) a polynucleotide comprising the nucleotide sequence of the full-length protein coding sequence of clone BD441_{—}1 deposited with the ATCC under accession number 98264;
(d) a polynucleotide encoding the full-length protein encoded by the cDNA insert of clone BD441_{—}1 deposited with the ATCC under accession number 98264;
(e) a polynucleotide comprising the nucleotide sequence of a mature protein coding sequence of clone BD441_{—}1 deposited with the ATCC under accession number 98264;
(f) a polynucleotide encoding a mature protein encoded by the cDNA insert of clone BD441_{—}1 deposited with the ATCC under accession number 98264;
(g) a polynucleotide encoding a protein comprising the amino acid sequence of SEQ ID NO:88;
(h) a polynucleotide encoding a protein comprising a fragment of the amino acid sequence of SEQ ID NO:88 having biological activity, the fragment comprising eight contiguous amino acids of SEQ ID NO:88;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:87.

Preferably, such polynucleotide comprises the nucleotide sequence of SEQ ID NO:87 from nucleotide 390 to nucleotide 563; the nucleotide sequence of the full-length protein coding sequence of clone BD441_{—}1 deposited with the ATCC under accession number 98264; or the nucleotide sequence of a mature protein coding sequence of clone BD441_{—}1 deposited with the ATCC under accession number 98264. In other preferred embodiments, the polynucleotide encodes the full-length or a mature protein encoded by the cDNA insert of clone BD441_{—}1 deposited with the ATCC under accession number 98264. In further preferred embodiments, the present invention provides a polynucleotide encoding a protein comprising a fragment of the amino acid sequence of SEQ ID NO:88 having biological activity, the fragment preferably comprising eight (more preferably twenty, most preferably thirty) contiguous amino acids of SEQ ID NO:88, or a polynucleotide encoding a protein comprising a fragment of the amino acid sequence of SEQ ID NO:88 having biological activity, the fragment comprising the amino acid sequence from amino acid 24 to amino acid 33 of SEQ ID NO:88.

Other embodiments provide the gene corresponding to the cDNA sequence of SEQ ID NO:87, SEQ ID NO:86, and SEQ ID NO:89. Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:86;
      (ab) SEQ ID NO:87;
      (ac) SEQ ID NO:89, but excluding the poly(A) tail at the 3′ end of SEQ ID NO:89; and
      (ad) the nucleotide sequence of the cDNA insert of clone BD441_{—}1 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:86;
      (bb) SEQ ID NO:87;
      (bc) SEQ ID NO:89, but excluding the poly(A) tail at the 3′ end of SEQ ID NO:89; and
      (bd) the nucleotide sequence of the cDNA insert of clone BD441_{—}1 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:86, SEQ ID NO:87, and SEQ ID NO:89, and extending contiguously from a nucleotide sequence corresponding to the 5′ end of SEQ ID NO:86 to a nucleotide sequence corresponding to the 3′ end of SEQ ID NO:89, but excluding the poly(A) tail at the 3′ end of SEQ ID NO:89. Also preferably the polynucleotide isolated according to the above process comprises a nucleotide sequence corresponding to the cDNA sequence of SEQ ID NO:87, and extending contiguously from a nucleotide sequence corresponding to the 5′ end of SEQ ID NO:87 to a nucleotide sequence corresponding to the 3′ end of SEQ ID NO:87. Also preferably the polynucleotide isolated according to the above process comprises a nucleotide sequence corresponding to the cDNA sequence of SEQ ID NO:87 from nucleotide 390 to nucleotide 563, and extending contiguously from a nucleotide sequence corresponding to the 5′ end of said sequence of SEQ ID NO:87 from nucleotide 390 to nucleotide 563, to a nucleotide sequence corresponding to the 3′ end of said sequence of SEQ ID NO:87 from nucleotide 390 to nucleotide 563.

In other embodiments, the present invention provides a composition comprising a protein, wherein said protein comprises an amino acid sequence selected from the group consisting of:
(a) the amino acid sequence of SEQ ID NO:88;
(b) a fragment of the amino acid sequence of SEQ ID NO:88, the fragment comprising eight contiguous amino acids of SEQ ID NO:88; and
(c) the amino acid sequence encoded by the cDNA insert of clone BD441_{—}1 deposited with the ATCC under accession number 98264;
   the protein being substantially free from other mammalian proteins. Preferably such protein comprises the amino acid sequence of SEQ ID NO:88. In further preferred embodiments, the present invention provides a protein comprising a fragment of the amino acid sequence of SEQ ID NO:88 having biological activity, the fragment preferably comprising eight (more preferably twenty, most preferably thirty) contiguous amino acids of SEQ ID NO:88, or a protein comprising a fragment of the amino acid sequence of SEQ ID NO:88 having biological activity, the fragment comprising the amino acid sequence from amino acid 24 to amino acid 33 of SEQ ID NO:88.

In one embodiment, the present invention provides a composition comprising an isolated polynucleotide selected from the group consisting of:
(a) a polynucleotide comprising the nucleotide sequence of SEQ ID NO:90;
(b) a polynucleotide comprising the nucleotide sequence of SEQ ID NO:90 from nucleotide 583 to nucleotide 756; (c) a polynucleotide comprising the nucleotide sequence of the full-length protein coding sequence of clone BD441_{—}2 deposited with the ATCC under accession number 98264;
(d) a polynucleotide encoding the full-length protein encoded by the cDNA insert of clone BD441_{—}2 deposited with the ATCC under accession number 98264;
(e) a polynucleotide comprising the nucleotide sequence of a mature protein coding sequence of clone BD441_{—}2 deposited with the ATCC under accession number 98264;
(f) a polynucleotide encoding a mature protein encoded by the cDNA insert of clone BD441_{—}2 deposited with the ATCC under accession number 98264;
(g) a polynucleotide encoding a protein comprising the amino acid sequence of SEQ ID NO:91;
(h) a polynucleotide encoding a protein comprising a fragment of the amino acid sequence of SEQ ID NO:91 having biological activity, the fragment comprising eight contiguous amino acids of SEQ ID NO:91;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:90.

Preferably, such polynucleotide comprises the nucleotide sequence of SEQ ID NO:90 from nucleotide 583 to nucleotide 756; the nucleotide sequence of the full-length protein coding sequence of clone BD441_{—}2 deposited with the ATCC under accession number 98264; or the nucleotide sequence of a mature protein coding sequence of clone BD441_{—}2 deposited with the ATCC under accession number 98264. In other preferred embodiments, the polynucleotide encodes the full-length or a mature protein encoded by the cDNA insert of clone BD441_{—}2 deposited with the ATCC under accession number 98264. In further preferred embodiments, the present invention provides a polynucleotide encoding a protein comprising a fragment of the amino acid sequence of SEQ ID NO:91 having biological activity, the fragment preferably comprising eight (more preferably twenty, most preferably thirty) contiguous amino acids of SEQ ID NO:91, or a polynucleotide encoding a protein comprising a fragment of the amino acid sequence of SEQ ID NO:91 having biological activity, the fragment comprising the amino acid sequence from amino acid 24 to amino acid 33 of SEQ ID NO:91.

Other embodiments provide the gene corresponding to the cDNA sequence of SEQ ID NO:90 and SEQ ID NO:92.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:90;
      (ab) SEQ ID NO:92, but excluding the poly(A) tail at the 3′ end of SEQ ID NO:92; and
      (ac) the nucleotide sequence of the cDNA insert of clone BD441_{—}2 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:90;
      (bb) SEQ ID NO:92, but excluding the poly(A) tail at the 3′ end of SEQ ID NO:92; and
      (bc) the nucleotide sequence of the cDNA insert of clone BD441_{—}2 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:90 and SEQ ID NO:92, and extending contiguously from a nucleotide sequence corresponding to the 5′ end of SEQ ID NO:90 to a nucleotide sequence corresponding to the 3′ end of SEQ ID NO:92, but excluding the poly(A) tail at the 3′ end of SEQ ID NO:92. Also preferably the polynucleotide isolated according to the above process comprises a nucleotide sequence corresponding to the cDNA sequence of SEQ ID NO:90, and extending contiguously from a nucleotide sequence corresponding to the 5′ end of SEQ ID NO:90 to a nucleotide sequence corresponding to the 3′ end of SEQ ID NO:90. Also preferably the polynucleotide isolated according to the above process comprises a nucleotide sequence corresponding to the cDNA sequence of SEQ ID NO:90 from nucleotide 583 to nucleotide 756, and extending contiguously from a nucleotide sequence corresponding to the 5′ end of said sequence of SEQ ID NO:90 from nucleotide 583 to nucleotide 756, to a nucleotide sequence corresponding to the 3′ end of said sequence of SEQ ID NO:90 from nucleotide 583 to nucleotide 756.

In other embodiments, the present invention provides a composition comprising a protein, wherein said protein comprises an amino acid sequence selected from the group consisting of:
(a) the amino acid sequence of SEQ ID NO:91;
(b) a fragment of the amino acid sequence of SEQ ID NO:91, the fragment comprising eight contiguous amino acids of SEQ ID NO:91; and
(c) the amino acid sequence encoded by the cDNA insert of clone BD441_{—}2 deposited with the ATCC under accession number 98264;
   the protein being substantially free from other mammalian proteins. Preferably such protein comprises the amino acid sequence of SEQ ID NO:91. In further preferred embodiments, the present invention provides a protein comprising a fragment of the amino add sequence of SEQ ID NO:91 having biological activity, the fragment preferably comprising eight (more preferably twenty, most preferably thirty) contiguous amino adds of SEQ ID NO:91, or a protein comprising a fragment of the amino acid sequence of SEQ ID NO:91 having biological activity, the fragment comprising the amino acid sequence from amino acid 24 to amino acid 33 of SEQ ID NO:91.

In one embodiment, the present invention provides a composition comprising an isolated polynucleotide selected from the group consisting of:
(a) a polynucleotide comprising the nucleotide sequence of SEQ ID NO:93;
(b) a polynucleotide comprising the nucleotide sequence of SEQ ID NO:93 from nucleotide 426 to nucleotide 581;
(c) a polynucleotide comprising the nucleotide sequence of SEQ ID NO:93 from nucleotide 495 to nucleotide 581;
(d) a polynucleotide comprising the nucleotide sequence of SEQ ID NO:93 from nucleotide 354 to nucleotide 503;
(e) a polynucleotide comprising the nucleotide sequence of the full-length protein coding sequence of clone BG102_{—}3 deposited with the ATCC under accession number 98264;
(f) a polynucleotide encoding the full-length protein encoded by the cDNA insert of clone BG102_{—}3 deposited with the ATCC under accession number 98264;
(g) a polynucleotide comprising the nucleotide sequence of a mature protein coding sequence of clone BG102_{—}3 deposited with the ATCC under accession number 98264;
(h) a polynucleotide encoding a mature protein encoded by the cDNA insert of clone BG102_{—}3 deposited with the ATCC under accession number 98264;
(i) a polynucleotide encoding a protein comprising the amino acid sequence of SEQ ID NO:94;
(j) a polynucleotide encoding a protein comprising a fragment of the amino acid sequence of SEQ ID NO:94 having biological activity, the fragment comprising eight contiguous amino acids of SEQ ID NO:94;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:93.

Preferably, such polynucleotide comprises the nucleotide sequence of SEQ ID NO:93 from nucleotide 426 to nucleotide 581; the nucleotide sequence of SEQ ID NO:93 from nucleotide 495 to nucleotide 581; the nucleotide sequence of SEQ ID NO:93 from nucleotide 354 to nucleotide 503; the nucleotide sequence of the full-length protein coding sequence of clone BG102_{—}3 deposited with the ATCC under accession number 98264; or the nucleotide sequence of a mature protein coding sequence of clone BG102_{—}3 deposited with the ATCC under accession number 98264. In other preferred embodiments, the polynucleotide encodes the full-length or a mature protein encoded by the cDNA insert of clone BG102_{—}3 deposited with the ATCC under accession number 98264. In yet other preferred embodiments, the present invention provides a polynucleotide encoding a protein comprising the amino acid sequence of SEQ ID NO:94 from amino acid 1 to amino acid 26. In further preferred embodiments, the present invention provides a polynucleotide encoding a protein comprising a fragment of the amino acid sequence of SEQ ID NO:94 having biological activity, the fragment preferably comprising eight (more preferably twenty, most preferably thirty) contiguous amino acids of SEQ ID NO:94, or a polynucleotide encoding a protein comprising a fragment of the amino acid sequence of SEQ ID NO:94 having biological activity, the fragment comprising the amino acid sequence from amino acid 21 to amino acid 30 of SEQ ID NO:94.

Other embodiments provide the gene corresponding to the cDNA sequence of SEQ ID NO:93.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:93, but excluding the poly(A) tail at the 3′ end of SEQ ID NO:93; and
      (ab) the nucleotide sequence of the cDNA insert of clone BG102_{—}3 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:93, but excluding the poly(A) tail at the 3′ end of SEQ ID NO:93; and
      (bb) the nucleotide sequence of the cDNA insert of clone BG102_{—}3 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:93, and extending contiguously from a nucleotide sequence corresponding to the 5′ end of SEQ ID NO:93 to a nucleotide sequence corresponding to the 3′ end of SEQ ID NO:93, but excluding the poly(A) tail at the 3′ end of SEQ ID NO:93. Also preferably the polynucleotide isolated according to the above process comprises a nucleotide sequence corresponding to the cDNA sequence of SEQ ID NO:93 from nucleotide 426 to nucleotide 581, and extending contiguously from a nucleotide sequence corresponding to the 5′ end of said sequence of SEQ ID NO:93 from nucleotide 426 to nucleotide 581, to a nucleotide sequence corresponding to the 3′ end of said sequence of SEQ ID NO:93 from nucleotide 426 to nucleotide 581. Also preferably the polynucleotide isolated according to the above process comprises a nucleotide sequence corresponding to the cDNA sequence of SEQ ID NO:93 from nucleotide 495 to nucleotide 581, and extending contiguously from a nucleotide sequence corresponding to the 5′ end of said sequence of SEQ ID NO:93 from nucleotide 495 to nucleotide 581, to a nucleotide sequence corresponding to the 3′ end of said sequence of SEQ ID NO:93 from nucleotide 495 to nucleotide 581. Also preferably the polynucleotide isolated according to the above process comprises a nucleotide sequence corresponding to the cDNA sequence of SEQ ID NO:93 from nucleotide 354 to nucleotide 503, and extending contiguously from a nucleotide sequence corresponding to the 5′ end of said sequence of SEQ ID NO:93 from nucleotide 354 to nucleotide 503, to a nucleotide sequence corresponding to the 3′ end of said sequence of SEQ ID NO:93 from nucleotide 354 to nucleotide 503.

In other embodiments, the present invention provides a composition comprising a protein, wherein said protein comprises an amino acid sequence selected from the group consisting of:
(a) the amino acid sequence of SEQ ID NO:94;
(b) the amino acid sequence of SEQ ID NO:94 from amino acid 1 to amino acid 26;
(c) a fragment of the amino acid sequence of SEQ ID NO:94, the fragment comprising eight contiguous amino acids of SEQ ID NO:94; and
(d) the amino acid sequence encoded by the cDNA insert of clone BG102_{—}3 deposited with the ATCC under accession number 98264;
the protein being substantially free from other mammalian proteins. Preferably such protein comprises the amino acid sequence of SEQ ID NO:94 or the amino acid sequence of SEQ ID NO:94 from amino acid 1 to amino acid 26. In further preferred embodiments, the present invention provides a protein comprising a fragment of the amino acid sequence of SEQ ID NO:94 having biological activity, the fragment preferably comprising eight (more preferably twenty, most preferably thirty) contiguous amino acids of SEQ ID NO:94, or a protein comprising a fragment of the amino acid sequence of SEQ ID NO:94 having biological activity, the fragment comprising the amino acid sequence from amino acid 21 to amino acid 30 of SEQ ID NO:94.

In one embodiment, the present invention provides a composition comprising an isolated polynucleotide selected from the group consisting of:
(a) a polynucleotide comprising the nucleotide sequence of SEQ ID NO:95;
(b) a polynucleotide comprising the nucleotide sequence of SEQ ID NO:95 from nucleotide 112 to nucleotide 978;
(c) a polynucleotide comprising the nucleotide sequence of SEQ ID NO:95 from nucleotide 436 to nucleotide 1048;
(d) a polynucleotide comprising the nucleotide sequence of the full-length protein coding sequence of clone BK158_{—}1 deposited with the ATCC under accession number 98264;
(e) a polynucleotide encoding the full-length protein encoded by the cDNA insert of clone BK158_{—}1 deposited with the ATCC under accession number 98264;
(f) a polynucleotide comprising the nucleotide sequence of a mature protein coding sequence of clone BK158_{—}1 deposited with the ATCC under accession number 98264;
(g) a polynucleotide encoding a mature protein encoded by the cDNA insert of clone BK158_{—}1 deposited with the ATCC under accession number 98264;
(h) a polynucleotide encoding a protein comprising the amino acid sequence of SEQ ID NO:96;
(i) a polynucleotide encoding a protein comprising a fragment of the amino add sequence of SEQ ID NO:96 having biological activity, the fragment comprising eight contiguous amino acids of SEQ ID NO:96;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:95.

Preferably, such polynucleotide comprises the nucleotide sequence of SEQ ID NO:95 from nucleotide 112 to nucleotide 978; the nucleotide sequence of SEQ ID NO:95 from nucleotide 436 to nucleotide 1048; the nucleotide sequence of the full-length protein coding sequence of clone BK158_{—}1 deposited with the ATCC under accession number 98264; or the nucleotide sequence of a mature protein coding sequence of clone BK158_{—}1 deposited with the ATCC under accession number 98264. In other preferred embodiments, the polynucleotide encodes the full-length or a mature protein encoded by the cDNA insert of clone BK158_{—}1 deposited with the ATCC under accession number 98264. In further preferred embodiments, the present invention provides a polynucleotide encoding a protein comprising a fragment of the amino acid sequence of SEQ ID NO:96 having biological activity, the fragment preferably comprising eight (more preferably twenty, most preferably thirty) contiguous amino acids of SEQ ID NO:96, or a polynucleotide encoding a protein comprising a fragment of the amino acid sequence of SEQ ID NO:96 having biological activity, the fragment comprising the amino acid sequence from amino acid 139 to amino acid 148 of SEQ ID NO:96.

Other embodiments provide the gene corresponding to the cDNA sequence of SEQ ID NO:95.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:95, but excluding the poly(A) tail at the 3′ end of SEQ ID NO:95; and
      (ab) the nucleotide sequence of the cDNA insert of clone BK158_{—}1 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:95, but excluding the poly(A) tail at the 3′ end of SEQ ID NO:95; and
      (bb) the nucleotide sequence of the cDNA insert of clone BK158_{—}1 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:95, and extending contiguously from a nucleotide sequence corresponding to the 5′ end of SEQ ID NO:95 to a nucleotide sequence corresponding to the 3′ end of SEQ ID NO:95, but excluding the poly(A) tail at the 3′ end of SEQ ID NO:95. Also preferably the polynucleotide isolated according to the above process comprises a nucleotide sequence corresponding to the cDNA sequence of SEQ ID NO:95 from nucleotide 112 to nucleotide 978, and extending contiguously from a nucleotide sequence corresponding to the 5′ end of said sequence of SEQ ID NO:95 from nucleotide 112 to nucleotide 978, to a nucleotide sequence corresponding to the 3′ end of said sequence of SEQ ID NO:95 from nucleotide 112 to nucleotide 978. Also preferably the polynucleotide isolated according to the above process comprises a nucleotide sequence corresponding to the cDNA sequence of SEQ ID NO:95 from nucleotide 436 to nucleotide 1048, and extending contiguously from a nucleotide sequence corresponding to the 5′ end of said sequence of SEQ ID NO:95 from nucleotide 436 to nucleotide 1048, to a nucleotide sequence corresponding to the 3′ end of said sequence of SEQ ID NO:95 from nucleotide 436 to nucleotide 1048.

In other embodiments, the present invention provides a composition comprising a protein, wherein said protein comprises an amino acid sequence selected from the group consisting of:
(a) the amino acid sequence of SEQ ID NO:96;
(b) a fragment of the amino acid sequence of SEQ ID NO:96, the fragment comprising eight contiguous amino acids of SEQ ID NO:96; and
(c) the amino acid sequence encoded by the cDNA insert of clone BK158_{—}1 deposited with the ATCC under accession number 98264;
   the protein being substantially free from other mammalian proteins. Preferably such protein comprises the amino acid sequence of SEQ ID NO:96. In further preferred embodiments, the present invention provides a protein comprising a fragment of the amino acid sequence of SEQ ID NO:96 having biological activity, the fragment preferably comprising eight (more preferably twenty, most preferably thirty) contiguous amino acids of SEQ ID NO:96, or a protein comprising a fragment of the amino acid sequence of SEQ ID NO:96 having biological activity, the fragment comprising the amino acid sequence from amino acid 139 to amino acid 148 of SEQ ID NO:96.

In one embodiment, the present invention provides a composition comprising an isolated polynucleotide selected from the group consisting of:
(a) a polynucleotide comprising the nucleotide sequence of SEQ ID NO:97;
(b) a polynucleotide comprising the nucleotide sequence of SEQ ID NO:97 from nucleotide 16 to nucleotide 492;
(c) a polynucleotide comprising the nucleotide sequence of the full-length protein coding sequence of clone BP163_{—}1 deposited with the ATCC under accession number 98264;
(d) a polynucleotide encoding the full-length protein encoded by the cDNA insert of clone BP163_{—}1 deposited with the ATCC under accession number 98264;
(e) a polynucleotide comprising the nucleotide sequence of a mature protein coding sequence of clone BP163_{—}1 deposited with the ATCC under accession number 98264;
(f) a polynucleotide encoding a mature protein encoded by the cDNA insert of clone BP163_{—}1 deposited with the ATCC under accession number 98264;
(g) a polynucleotide encoding a protein comprising the amino acid sequence of SEQ ID NO:98;
(h) a polynucleotide encoding a protein comprising a fragment of the amino acid sequence of SEQ ID NO:98 having biological activity, the fragment comprising eight contiguous amino acids of SEQ ID NO:98;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:97.

Preferably, such polynucleotide comprises the nucleotide sequence of SEQ ID NO:97 from nucleotide 16 to nucleotide 492; the nucleotide sequence of the full-length protein coding sequence of clone BP163_{—}1 deposited with the ATCC under accession number 98264; or the nucleotide sequence of a mature protein coding sequence of clone BP163_{—}1 deposited with the ATCC under accession number 98264. In other preferred embodiments, the polynucleotide encodes the full-length or a mature protein encoded by the cDNA insert of clone BP163_{—}1 deposited with the ATCC under accession number 98264. In further preferred embodiments, the present invention provides a polynucleotide encoding a protein comprising a fragment of the amino acid sequence of SEQ ID NO:98 having biological activity, the fragment preferably comprising eight (more preferably twenty, most preferably thirty) contiguous amino acids of SEQ ID NO:98, or a polynucleotide encoding a protein comprising a fragment of the amino acid sequence of SEQ ID NO:98 having biological activity, the fragment comprising the amino acid sequence from amino acid 74 to amino acid 83 of SEQ ID NO:98.

Other embodiments provide the gene corresponding to the cDNA sequence of SEQ ID NO:97 and SEQ ID NO:99.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:97;
      (ab) SEQ ID NO:99, but excluding the poly(A) tail at the 3′ end of SEQ ID NO:99; and
      (ac) the nucleotide sequence of the cDNA insert of clone BP163_{—}1 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:97;
      (bb) SEQ ID NO:99, but excluding the poly(A) tail at the 3′ end of SEQ ID NO:99; and
      (bc) the nucleotide sequence of the cDNA insert of clone BP163_{—}1 deposited with the ATCC under accession number 98264;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:97 and SEQ ID NO:99, and extending contiguously from a nucleotide sequence corresponding to the 5′ end of SEQ ID NO:97 to a nucleotide sequence corresponding to the 3′ end of SEQ ID NO:99, but excluding the poly(A) tail at the 3′ end of SEQ ID NO:99. Also preferably the polynucleotide isolated according to the above process comprises a nucleotide sequence corresponding to the cDNA sequence of SEQ ID NO:97, and extending contiguously from a nucleotide sequence corresponding to the 5′ end of SEQ ID NO:97 to a nucleotide sequence corresponding to the 3′ end of SEQ ID NO:97. Also preferably the polynucleotide isolated according to the above process comprises a nucleotide sequence corresponding to the cDNA sequence of SEQ ID NO:97 from nucleotide 16 to nucleotide 492, and extending contiguously from a nucleotide sequence corresponding to the 5′ end of said sequence of SEQ ID NO:97 from nucleotide 16 to nucleotide 492, to a nucleotide sequence corresponding to the 3′ end of said sequence of SEQ ID NO:97 from nucleotide 16 to nucleotide 492.

In other embodiments, the present invention provides a composition comprising a protein, wherein said protein comprises an amino acid sequence selected from the group consisting of:
(a) the amino acid sequence of SEQ ID NO:98;
(b) a fragment of the amino acid sequence of SEQ ID NO:98, the fragment comprising eight contiguous amino acids of SEQ ID NO:98; and
(c) the amino acid sequence encoded by the cDNA insert of clone BP163_{—}1 deposited with the ATCC under accession number 98264;
   the protein being substantially free from other mammalian proteins. Preferably such protein comprises the amino acid sequence of SEQ ID NO:98. In further preferred embodiments, the present invention provides a protein comprising a fragment of the amino acid sequence of SEQ ID NO:98 having biological activity, the fragment preferably comprising eight (more preferably twenty, most preferably thirty) contiguous amino acids of SEQ ID NO:98, or a protein comprising a fragment of the amino acid sequence of SEQ ID NO:98 having biological activity, the fragment comprising the amino acid sequence from amino acid 74 to amino acid 83 of SEQ ID NO:98.

In one embodiment, the present invention provides a composition comprising an isolated polynucleotide selected from the group consisting of:
(a) a polynucleotide comprising the nucleotide sequence of SEQ ID NO:101;
(b) a polynucleotide comprising the nucleotide sequence of SEQ ID NO:101 from nucleotide 72 to nucleotide 569;
(c) a polynucleotide comprising the nucleotide sequence of the full-length protein coding sequence of clone BZ16_{—}3 deposited with the ATCC under accession number 98264;
(d) a polynucleotide encoding the full-length protein encoded by the cDNA insert of clone BZ16_{—}3 deposited with the ATCC under accession number 98264;
(e) a polynucleotide comprising the nucleotide sequence of a mature protein coding sequence of clone BZ163 deposited with the ATCC under accession number 98264;
(f) a polynucleotide encoding a mature protein encoded by the cDNA insert of clone BZ6_{—}3 deposited with the ATCC under accession number 98264;
(g) a polynucleotide encoding a protein comprising the amino acid sequence of SEQ ID NO:102;
(h) a polynucleotide encoding a protein comprising a fragment of the amino acid sequence of SEQ ID NO:102 having biological activity, the fragment comprising eight contiguous amino adds of SEQ ID NO:102;
(i) a polynucleotide which is an allelic variant of a polynucleotide of (a>(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:101.

Preferably, such polynucleotide comprises the nucleotide sequence of SEQ ID NO:101 from nucleotide 72 to nucleotide 569; the nucleotide sequence of the full-length protein coding sequence of clone BZ16_{—}3 deposited with the ATCC under accession number 98264; or the nucleotide sequence of a mature protein coding sequence of clone BZ16_{—}3 deposited with the ATCC under accession number 98264. In other preferred embodiments, the polynucleotide encodes the full-length or a mature protein encoded by the cDNA insert of clone BZ16_{—}3 deposited with the ATCC under accession number 98264. In yet other preferred embodiments, the present invention provides a polynucleotide encoding a protein comprising the amino acid sequence of SEQ ID NO:102 from amino acid 1 to amino acid 124. In further preferred embodiments, the present invention provides a polynucleotide encoding a protein comprising a fragment of the amino acid sequence of SEQ ID NO:102 having biological activity, the fragment preferably comprising eight (more preferably twenty, most preferably thirty) contiguous amino adds of SEQ ID NO:102, or a polynucleotide encoding a protein comprising a fragment of the amino acid sequence of SEQ ID NO:102 having biological activity, the fragment comprising the amino acid sequence from amino acid 78 to amino acid 87 of SEQ ID NO:102.

Other embodiments provide the gene corresponding to the cDNA sequence of SEQ ID NO:101 and SEQ ID NO:100.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:100;
      (ab) SEQ ID NO:101, but excluding the poly(A) tail at the 3′ end of SEQ ID NO:101; and
      (ac) the nucleotide sequence of the cDNA insert of clone BZ16_{—}3 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:100;
      (bb) SEQ ID NO:101, but excluding the poly(A) tail at the 3′ end of SEQ ID NO:101; and
      (bc) the nucleotide sequence of the cDNA insert of clone BZ16_{—}3 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:100 and SEQ ID NO:101, and extending contiguously from a nucleotide sequence corresponding to the 5′ end of SEQ ID NO:100 to a nucleotide sequence corresponding to the 3′ end of SEQ ID NO:101, but excluding the poly(A) tail at the 3′ end of SEQ ID NO:101. Also preferably the polynucleotide isolated according to the above process comprises a nucleotide sequence corresponding to the cDNA sequence of SEQ ID NO:101, and extending contiguously from a nucleotide sequence corresponding to the 5′ end of SEQ ID NO:101 to a nucleotide sequence corresponding to the 3′ end of SEQ ID NO:101, but excluding the poly(A) tail at the 3′ end of SEQ ID NO:101. Also preferably the polynucleotide isolated according to the above process comprises a nucleotide sequence corresponding to the cDNA sequence of SEQ ID NO:101 from nucleotide 72 to nucleotide 569, and extending contiguously from a nucleotide sequence corresponding to the 5′ end of said sequence of SEQ ID NO:101 from nucleotide 72 to nucleotide 569, to a nucleotide sequence corresponding to the 3′ end of said sequence of SEQ ID NO:101 from nucleotide 72 to nucleotide 569.

In other embodiments, the present invention provides a composition comprising a protein, wherein said protein comprises an amino acid sequence selected from the group consisting of:
(a) the amino acid sequence of SEQ ID NO:102;
(b) the amino acid sequence of SEQ ID NO:102 from amino acid 1 to amino acid 124;
(c) a fragment of the amino acid sequence of SEQ ID NO:102, the fragment comprising eight contiguous amino acids of SEQ ID NO:102; and
(d) the amino acid sequence encoded by the cDNA insert of clone BZ16_{—}3 deposited with the ATCC under accession number 98264;
   the protein being substantially free from other mammalian proteins. Preferably such protein comprises the amino acid sequence of SEQ ID NO:102 or the amino acid sequence of SEQ ID NO:102 from amino acid 1 to amino acid 124. In further preferred embodiments, the present invention provides a protein comprising a fragment of the amino acid sequence of SEQ ID NO:102 having biological activity, the fragment preferably comprising eight (more preferably twenty, most preferably thirty) contiguous amino adds of SEQ ID NO:102, or a protein comprising a fragment of the amino acid sequence of SEQ ID NO:102 having biological activity, the fragment comprising the amino acid sequence from amino acid 78 to amino acid 87 of SEQ ID NO:102.

In one embodiment, the present invention provides a composition comprising an isolated polynucleotide selected from the group consisting of:
(a) a polynucleotide comprising the nucleotide sequence of SEQ ID NO:103;
(b) a polynucleotide comprising the nucleotide sequence of SEQ ID NO:103 from nucleotide 405 to nucleotide 662;
(c) a polynucleotide comprising the nucleotide sequence of SEQ ID NO:103 from nucleotide 519 to nucleotide 662;
(d) a polynucleotide comprising the nucleotide sequence of SEQ ID NO:103 from nucleotide 1 to nucleotide 584;
(e) a polynucleotide comprising the nucleotide sequence of the full-length protein coding sequence of clone CC182_{—}1 deposited with the ATCC under accession number 98264;
(f) a polynucleotide encoding the full-length protein encoded by the cDNA insert of clone CC182_{—}1 deposited with the ATCC under accession number 98264;
(g) a polynucleotide comprising the nucleotide sequence of a mature protein coding sequence of clone CC182_{—}1 deposited with the ATCC under accession number 98264;
(h) a polynucleotide encoding a mature protein encoded by the cDNA insert of clone CC182_{—}1 deposited with the ATCC under accession number 98264;
(i) a polynucleotide encoding a protein comprising the amino acid sequence of SEQ ID NO:104;
a polynucleotide encoding a protein comprising a fragment of the amino acid sequence of SEQ ID NO:104 having biological activity, the fragment comprising eight contiguous amino acids of SEQ ID NO:104;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:103.

Preferably, such polynucleotide comprises the nucleotide sequence of SEQ ID NO:103 from nucleotide 405 to nucleotide 662; the nucleotide sequence of SEQ ID NO:103 from nucleotide 519 to nucleotide 662; the nucleotide sequence of SEQ ID NO:103 from nucleotide 1 to nucleotide 584; the nucleotide sequence of the full-length protein coding sequence of clone CC182_{—}1 deposited with the ATCC under accession number 98264; or the nucleotide sequence of a mature protein coding sequence of clone CC182_{—}1 deposited with the ATCC under accession number 93264. In other preferred embodiments, the polynucleotide encodes the full-length or a mature protein encoded by the cDNA insert of clone CC182_{—}1 deposited with the ATCC under accession number 98264. In yet other preferred embodiments, the present invention provides a polynucleotide encoding a protein comprising the amino acid sequence of SEQ ID NO:104 from amino acid 1 to amino acid 60. In further preferred embodiments, the present invention provides a polynucleotide encoding a protein comprising a fragment of the amino acid sequence of SEQ ID NO:104 having biological activity, the fragment preferably comprising eight (more preferably twenty, most preferably thirty) contiguous amino acids of SEQ ID NO:104, or a polynucleotide encoding a protein comprising a fragment of the amino acid sequence of SEQ ID NO:104 having biological activity, the fragment comprising the amino acid sequence from amino acid 38 to amino acid 47 of SEQ ID NO:104. Other embodiments provide the gene corresponding to the cDNA sequence of SEQ ID NO:103.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:103, but excluding the poly(A) tail at the 3′ end of SEQ ID NO:103; and
      (ab) the nucleotide sequence of the cDNA insert of clone CC182_{—}1 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:103, but excluding the poly(A) tail at the 3′ end of SEQ ID NO:103; and
      (bb) the nucleotide sequence of the cDNA insert of clone CC182_{—}1 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:103, and extending contiguously from a nucleotide sequence corresponding to the 5′ end of SEQ ID NO:103 to a nucleotide sequence corresponding to the 3′ end of SEQ ID NO:103, but excluding the poly(A) tail at the 3′ end of SEQ ID NO:103. Also preferably the polynucleotide isolated according to the above process comprises a nucleotide sequence corresponding to the cDNA sequence of SEQ ID NO:103 from nucleotide 405 to nucleotide 662, and extending contiguously from a nucleotide sequence corresponding to the 5′ end of said sequence of SEQ ID NO:103 from nucleotide 405 to nucleotide 662, to a nucleotide sequence corresponding to the 3′ end of said sequence of SEQ ID NO:103 from nucleotide 405 to nucleotide 662. Also preferably the polynucleotide isolated according to the above process comprises a nucleotide sequence corresponding to the cDNA sequence of SEQ ID NO:103 from nucleotide 519 to nucleotide 662, and extending contiguously from a nucleotide sequence corresponding to the 5′ end of said sequence of SEQ ID NO:103 from nucleotide 519 to nucleotide 662, to a nucleotide sequence corresponding to the 3′ end of said sequence of SEQ ID NO:103 from nucleotide 519 to nucleotide 662. Also preferably the polynucleotide isolated according to the above process comprises a nucleotide sequence corresponding to the cDNA sequence of SEQ ID NO:103 from nucleotide 1 to nucleotide 584, and extending contiguously from a nucleotide sequence corresponding to the 5′ end of said sequence of SEQ ID NO:103 from nucleotide 1 to nucleotide 584, to a nucleotide sequence corresponding to the 3′ end of said sequence of SEQ ID NO:103 from nucleotide 1 to nucleotide 584.

In other embodiments, the present invention provides a composition comprising a protein, wherein said protein comprises an amino acid sequence selected from the group consisting of:
(a) the amino acid sequence of SEQ ID NO:104;
(b) the amino acid sequence of SEQ ID NO:104 from amino acid 1 to amino acid 60;
(c) a fragment of the amino acid sequence of SEQ ID NO:104, the fragment comprising eight contiguous amino acids of SEQ ID NO:104; and
(d) the amino acid sequence encoded by the cDNA insert of clone CC182_{—}1 deposited with the ATCC under accession number 98264;
   the protein being substantially free from other mammalian proteins. Preferably such protein comprises the amino acid sequence of SEQ ID NO:104 or the amino acid sequence of SEQ ID NO:104 from amino acid 1 to amino acid 60. In further preferred embodiments, the present invention provides a protein comprising a fragment of the amino acid sequence of SEQ ID NO:104 having biological activity, the fragment preferably comprising eight (more preferably twenty, most preferably thirty) contiguous amino acids of SEQ ID NO:104, or a protein comprising a fragment of the amino acid sequence of SEQ ID NO:104 having biological activity, the fragment comprising the amino acid sequence from amino acid 38 to amino acid 47 of SEQ ID NO:104.

In one embodiment, the present invention provides a composition comprising an isolated polynucleotide selected from the group consisting of:
(a) a polynucleotide comprising the nucleotide sequence of SEQ ID NO:105;
(b) a polynucleotide comprising the nucleotide sequence of SEQ ID NO:105 from nucleotide 311 to nucleotide 409;
(c) a polynucleotide comprising the nucleotide sequence of SEQ ID NO:105 from nucleotide 24 to nucleotide 414;
(d) a polynucleotide comprising the nucleotide sequence of the full-length protein coding sequence of clone CG109_{—}1 deposited with the ATCC under accession number 98264;
(e) a polynucleotide encoding the full-length protein encoded by the cDNA insert of clone CG109_{—}1 deposited with the ATCC under accession number 98264;
(f) a polynucleotide comprising the nucleotide sequence of a mature protein coding sequence of clone CG109_{—}1 deposited with the ATCC under accession number 98264;
(g) a polynucleotide encoding a mature protein encoded by the cDNA insert of clone CG109_{—}1 deposited with the ATCC under accession number 98264;
(h) a polynucleotide encoding a protein comprising the amino acid sequence of SEQ ID NO:106;
(i) a polynucleotide encoding a protein comprising a fragment of the amino acid sequence of SEQ ID NO:106 having biological activity, the fragment comprising eight contiguous amino acids of SEQ ID NO:106;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:105.

Preferably, such polynucleotide comprises the nucleotide sequence of SEQ ID NO:105 from nucleotide 311 to nucleotide 409; the nucleotide sequence of SEQ ID NO:105 from nucleotide 24 to nucleotide 414; the nucleotide sequence of the full-length protein coding sequence of clone CG109_{—}1 deposited with the ATCC under accession number 98264; or the nucleotide sequence of a mature protein coding sequence of clone CG109_{—}1 deposited with the ATCC under accession number 98264. In other preferred embodiments, the polynucleotide encodes the full-length or a mature protein encoded by the cDNA insert of clone CG109_{—}1 deposited with the ATCC under accession number 98264. In further preferred embodiments, the present invention provides a polynucleotide encoding a protein comprising a fragment of the amino acid sequence of SEQ ID NO:106 having biological activity, the fragment preferably comprising eight (more preferably twenty, most preferably thirty) contiguous amino acids of SEQ ID NO:106, or a polynucleotide encoding a protein comprising a fragment of the amino acid sequence of SEQ ID NO:106 having biological activity, the fragment comprising the amino acid sequence from amino acid 11 to amino acid 20 of SEQ ID NO:106.

Other embodiments provide the gene corresponding to the cDNA sequence of SEQ ID NO:105.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:105, but excluding the poly(A) tail at the 3′ end of SEQ ID NO:105; and
      (ab) the nucleotide sequence of the cDNA insert of clone CG109_{—}1 deposited with the ATCC under accession number 98264;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO 105, but excluding the poly(A) tail at the 3′ end of SEQ ID NO:105; and
      (bb) the nucleotide sequence of the cDNA insert of clone CG109_{—}1 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:105, and extending contiguously from a nucleotide sequence corresponding to the 5′ end of SEQ ID NO 105 to a nucleotide sequence corresponding to the 3′ end of SEQ ID NO:105, but excluding the poly(A) tail at the 3′ end of SEQ ID NO:105. Also preferably the polynucleotide isolated according to the above process comprises a nucleotide sequence corresponding to the cDNA sequence of SEQ ID NO:105 from nucleotide 311 to nucleotide 409, and extending contiguously from a nucleotide sequence corresponding to the 5′ end of said sequence of SEQ ID NO:105 from nucleotide 311 to nucleotide 409, to a nucleotide sequence corresponding to the 3′ end of said sequence of SEQ ID NO:105 from nucleotide 311 to nucleotide 409. Also preferably the polynucleotide isolated according to the above process comprises a nucleotide sequence corresponding to the cDNA sequence of SEQ ID NO:105 from nucleotide 24 to nucleotide 414, and extending contiguously from a nucleotide sequence corresponding to the 5′ end of said sequence of SEQ ID NO:105 from nucleotide 24 to nucleotide 414, to a nucleotide sequence corresponding to the 3′ end of said sequence of SEQ ID NO:105 from nucleotide 24 to nucleotide 414.

In other embodiments, the present invention provides a composition comprising a protein, wherein said protein comprises an amino acid sequence selected from the group consisting of:
(a) the amino acid sequence of SEQ ID NO:106;
(b) a fragment of the amino acid sequence of SEQ ID NO:106, the fragment comprising eight contiguous amino acids of SEQ ID NO:106; and
(c) the amino acid sequence encoded by the cDNA insert of clone CG109_{—}1 deposited with the ATCC under accession number 98264;
   the protein being substantially free from other mammalian proteins. Preferably such protein comprises the amino acid sequence of SEQ ID NO:106. In further preferred embodiments, the present invention provides a protein comprising a fragment of the amino acid sequence of SEQ ID NO:106 having biological activity, the fragment preferably comprising eight (more preferably twenty, most preferably thirty) contiguous amino acids of SEQ ID NO:106, or a protein comprising a fragment of the amino acid sequence of SEQ ID NO:106 having biological activity, the fragment comprising the amino acid sequence from amino acid 11 to amino acid 20 of SEQ ID NO:106.

In one embodiment, the present invention provides a composition comprising an isolated polynucleotide selected from the group consisting of:
(a) a polynucleotide comprising the nucleotide sequence of SEQ ID NO:108;
(b) a polynucleotide comprising the nucleotide sequence of SEQ ID NO:108 from nucleotide 471 to nucleotide 611;
(c) a polynucleotide comprising the nucleotide sequence of the full-length protein coding sequence of clone CJ397_{—}1 deposited with the ATCC under accession number 98264;
(d) a polynucleotide encoding the full-length protein encoded by the cDNA insert of clone CJ397_{—}1 deposited with the ATCC under accession number 98264;
(e) a polynucleotide comprising the nucleotide sequence of a mature protein coding sequence of clone CJ397_{—}1 deposited with the ATCC under accession number 98264;
(f) a polynucleotide encoding a mature protein encoded by the cDNA insert of clone CJ397_{—}1 deposited with the ATCC under accession number 98264;
(g) a polynucleotide encoding a protein comprising the amino acid sequence of SEQ ID NO:109;
(h) a polynucleotide encoding a protein comprising a fragment of the amino acid sequence of SEQ ID NO:109 having biological activity, the fragment comprising eight contiguous amino acids of SEQ ID NO:109;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:108.

Preferably, such polynucleotide comprises the nucleotide sequence of SEQ ID NO:108 from nucleotide 471 to nucleotide 611; the nucleotide sequence of the full-length protein coding sequence of clone CJ397_{—}1 deposited with the ATCC under accession number 98264; or the nucleotide sequence of a mature protein coding sequence of clone CJ397_{—}1 deposited with the ATCC under accession number 98264. In other preferred embodiments, the polynucleotide encodes the full-length or a mature protein encoded by the cDNA insert of clone CJ397_{—}1 deposited with the ATCC under accession number 98264. In further preferred embodiments, the present invention provides a polynucleotide encoding a protein comprising a fragment of the amino acid sequence of SEQ ID NO:109 having biological activity, the fragment preferably comprising eight (more preferably twenty, most preferably thirty) contiguous amino adds of SEQ ID NO:109, or a polynucleotide encoding a protein comprising a fragment of the amino acid sequence of SEQ ID NO:109 having biological activity, the fragment comprising the amino acid sequence from amino acid 18 to amino acid 27 of SEQ ID NO:109.

Other embodiments provide the gene corresponding to the cDNA sequence of SEQ ID NO:108, SEQ ID NO:107, and SEQ ID NO:110.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of
      (aa) SEQ ID NO:107;
      (ab) SEQ ID NO:108;
      (ac) SEQ ID NO:110, but excluding the poly(A) tail at the 3′ end of SEQ ID NO:110; and
      (ad) the nucleotide sequence of the cDNA insert of clone CJ397_{—}1 deposited with the ATCC under accession number 98264;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:107;
      (bb) SEQ ID NO:108;
      (bc) SEQ ID NO:110, but excluding the poly(A) tail at the 3′ end of SEQ ID NO:110; and
      (bd) the nucleotide sequence of the cDNA insert of clone CJ397_{—}1 deposited with the ATCC under accession number 98264;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:107, SEQ ID NO:108, and SEQ ID NO:110, and extending contiguously from a nucleotide sequence corresponding to the 5′ end of SEQ ID NO:107 to a nucleotide sequence corresponding to the 3′ end of SEQ ID NO:110, but excluding the poly(A) tail at the 3′ end of SEQ ID NO:110. Also preferably the polynucleotide isolated according to the above process comprises a nucleotide sequence corresponding to the cDNA sequence of SEQ ID NO:108, and extending contiguously from a nucleotide sequence corresponding to the 5′ end of SEQ ID NO:108 to a nucleotide sequence corresponding to the 3′ end of SEQ ID NO:108. Also preferably the polynucleotide isolated according to the above process comprises a nucleotide sequence corresponding to the cDNA sequence of SEQ ID NO:108 from nucleotide 471 to nucleotide 611, and extending contiguously from a nucleotide sequence corresponding to the 5′ end of said sequence of SEQ ID NO:108 from nucleotide 471 to nucleotide 611, to a nucleotide sequence corresponding to the 3′ end of said sequence of SEQ ID NO:108 from nucleotide 471 to nucleotide 611.

In other embodiments, the present invention provides a composition comprising a protein, wherein said protein comprises an amino acid sequence selected from the group consisting of:
(a) the amino acid sequence of SEQ ID NO:109;
(b) a fragment of the amino acid sequence of SEQ ID NO:109, the fragment comprising eight contiguous amino acids of SEQ ID NO:109; and
(c) the amino acid sequence encoded by the cDNA insert of clone CJ397_{—}1 deposited with the ATCC under accession number 98264;
   the protein being substantially free from other mammalian proteins. Preferably such protein comprises the amino acid sequence of SEQ ID NO:109. In further preferred embodiments, the present invention provides a protein comprising a fragment of the amino acid sequence of SEQ ID NO:109 having biological activity, the fragment preferably comprising eight (more preferably twenty, most preferably thirty) contiguous amino acids of SEQ ID NO:109, or a protein comprising a fragment of the amino acid sequence of SEQ ID NO:109 having biological activity, the fragment comprising the amino acid sequence from amino acid 18 to amino acid 27 of SEQ ID NO:109.

In one embodiment, the present invention provides a composition comprising an isolated polynucleotide selected from the group consisting of:
(a) a polynucleotide comprising the nucleotide sequence of SEQ ID NO:111;
(b) a polynucleotide comprising the nucleotide sequence of SEQ ID NO:111 from nucleotide 141 to nucleotide 1532;
(c) a polynucleotide comprising the nucleotide sequence of SEQ ID NO:111 from nucleotide 204 to nucleotide 1532;
(d) a polynucleotide comprising the nucleotide sequence of SEQ ID NO:111 from nucleotide 78 to nucleotide 476;
(e) a polynucleotide comprising the nucleotide sequence of the full-length protein coding sequence of clone AM795_{—}4 deposited with the ATCC under accession number 98271;
(f) a polynucleotide encoding the full-length protein encoded by the cDNA insert of clone AM795_{—}4 deposited with the ATCC under accession number 98271;
(g) a polynucleotide comprising the nucleotide sequence of a mature protein coding sequence of clone AM795_{—}4 deposited with the ATCC under accession number 98271;
(h) a polynucleotide encoding a mature protein encoded by the cDNA insert of clone AM795_{—}4 deposited with the ATCC under accession number 98271;
(i) a polynucleotide encoding a protein comprising the amino acid sequence of SEQ ID NO:112;
(j) a polynucleotide encoding a protein comprising a fragment of the amino acid sequence of SEQ ID NO:112 having biological activity, the fragment comprising eight contiguous amino acids of SEQ ID NO:112;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:111.

Preferably, such polynucleotide comprises the nucleotide sequence of SEQ ID NO:111 from nucleotide 141 to nucleotide 1532; the nucleotide sequence of SEQ ID NO:111 from nucleotide 204 to nucleotide 1532; the nucleotide sequence of SEQ ID NO:111 from nucleotide 78 to nucleotide 476; the nucleotide sequence of the full-length protein coding sequence of clone AM795_{—}4 deposited with the ATCC under accession number 98271; or the nucleotide sequence of a mature protein coding sequence of clone AM795_{—}4 deposited with the ATCC under accession number 98271. In other preferred embodiments, the polynucleotide encodes the full-length or a mature protein encoded by the cDNA insert of clone AM795_{—}4 deposited with the ATCC under accession number 98271. In yet other preferred embodiments, the present invention provides a polynucleotide encoding a protein comprising the amino acid sequence of SEQ ID NO:112 from amino acid 1 to amino acid 112. In further preferred embodiments, the present invention provides a polynucleotide encoding a protein comprising a fragment of the amino acid sequence of SEQ ID NO:112 having biological activity, the fragment preferably comprising eight (more preferably twenty, most preferably thirty) contiguous amino adds of SEQ ID NO:112, or a polynucleotide encoding a protein comprising a fragment of the amino acid sequence of SEQ ID NO:112 having biological activity, the fragment comprising the amino acid sequence from amino acid 227 to amino acid 236 of SEQ ID NO:112.

Other embodiments provide the gene corresponding to the cDNA sequence of SEQ ID NO:111.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:111, but excluding the poly(A) tail at the 3′ end of SEQ ID NO:111; and
      (ab) the nucleotide sequence of the cDNA insert of clone AM795_{—}4 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:111, but excluding the poly(A) tail at the 3′ end of SEQ ID NO:111; and
      (bb) the nucleotide sequence of the cDNA insert of clone AM795_{—}4 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:111, and extending contiguously from a nucleotide sequence corresponding to the 5′ end of SEQ ID NO:111 to a nucleotide sequence corresponding to the 3′ end of SEQ ID NO:111, but excluding the poly(A) tail at the 3′ end of SEQ ID NO:111. Also preferably the polynucleotide isolated according to the above process comprises a nucleotide sequence corresponding to the cDNA sequence of SEQ ID NO:111 from nucleotide 141 to nucleotide 1532, and extending contiguously from a nucleotide sequence corresponding to the 5′ end of said sequence of SEQ ID NO:111 from nucleotide 141 to nucleotide 1532, to a nucleotide sequence corresponding to the 3′ end of said sequence of SEQ ID NO:111 from nucleotide 141 to nucleotide 1532. Also preferably the polynucleotide isolated according to the above process comprises a nucleotide sequence corresponding to the cDNA sequence of SEQ ID NO:111 from nucleotide 204 to nucleotide 1532, and extending contiguously from a nucleotide sequence corresponding to the 5′ end of said sequence of SEQ ID NO:111 from nucleotide 204 to nucleotide 1532, to a nucleotide sequence corresponding to the 3′ end of said sequence of SEQ ID NO:111 from nucleotide 204 to nucleotide 1532. Also preferably the polynucleotide isolated according to the above process comprises a nucleotide sequence corresponding to the cDNA sequence of SEQ ID NO:111 from nucleotide 78 to nucleotide 476, and extending contiguously from a nucleotide sequence corresponding to the 5′ end of said sequence of SEQ ID NO:111 from nucleotide 78 to nucleotide 476, to a nucleotide sequence corresponding to the 3′ end of said sequence of SEQ ID NO:111 from nucleotide 78 to nucleotide 476.

In other embodiments, the present invention provides a composition comprising a protein, wherein said protein comprises an amino acid sequence selected from the group consisting of:
(a) the amino acid sequence of SEQ ID NO:112;
(b) the amino acid sequence of SEQ ID NO:112 from amino acid 1 to amino acid 112;
(c) a fragment of the amino acid sequence of SEQ ID NO:112, the fragment comprising eight contiguous amino acids of SEQ ID NO:112; and
(d) the amino acid sequence encoded by the cDNA insert of clone AM795_{—}4 deposited with the ATCC under accession number 98271;
   the protein being substantially free from other mammalian proteins. Preferably such protein comprises the amino add sequence of SEQ ID NO:112 or the amino acid sequence of SEQ ID NO:112 from amino acid 1 to amino acid 112. In further preferred embodiments, the present invention provides a protein comprising a fragment of the amino acid sequence of SEQ ID NO 112 having biological activity, the fragment preferably comprising eight (more preferably twenty, most preferably thirty) contiguous amino acids of SEQ ID NO:112, or a protein comprising a fragment of the amino acid sequence of SEQ ID NO:112 having biological activity, the fragment comprising the amino acid sequence from amino acid 227 to amino acid 236 of SEQ ID NO:112.

In one embodiment, the present invention provides a composition comprising an isolated polynucleotide selected from the group consisting of:
(a) a polynucleotide comprising the nucleotide sequence of SEQ ID NO:114;
(b) a polynucleotide comprising the nucleotide sequence of SEQ ID NO:114 from nucleotide 19 to nucleotide 262;
(c) a polynucleotide comprising the nucleotide sequence of SEQ ID NO:114 from nucleotide 91 to nucleotide 262;
(d) a polynucleotide comprising the nucleotide sequence of the full-length protein coding sequence of clone AT340_{—}1 deposited with the ATCC under accession number 98271;
(e) a polynucleotide encoding the full-length protein encoded by the cDNA insert of clone AT340_{—}1 deposited with the ATCC under accession number 98271;
(f) a polynucleotide comprising the nucleotide sequence of a mature protein coding sequence of clone AT340_{—}1 deposited with the ATCC under accession number 98271;
(g) a polynucleotide encoding a mature protein encoded by the cDNA insert of clone AT340_{—}1 deposited with the ATCC under accession number 98271;
(h) a polynucleotide encoding a protein comprising the amino acid sequence of SEQ ID NO:115;
(i) a polynucleotide encoding a protein comprising a fragment of the amino acid sequence of SEQ ID NO:115 having biological activity, the fragment comprising eight contiguous amino acids of SEQ ID NO:115;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:114.

Preferably, such polynucleotide comprises the nucleotide sequence of SEQ ID NO:114 from nucleotide 19 to nucleotide 262; the nucleotide sequence of SEQ ID NO:114 from nucleotide 91 to nucleotide 262; the nucleotide sequence of the full-length protein coding sequence of clone AT340_{—}1 deposited with the ATCC under accession number 98271; or the nucleotide sequence of a mature protein coding sequence of clone AT340_{—}1 deposited with the ATCC under accession number 98271. In other preferred embodiments, the polynucleotide encodes the full-length or a mature protein encoded by the cDNA insert of clone AT340_{—}1 deposited with the ATCC under accession number 98271. In yet other preferred embodiments, the present invention provides a polynucleotide encoding a protein comprising the amino acid sequence of SEQ ID NO:115 from amino acid 1 to amino acid 66. In further preferred embodiments, the present invention provides a polynucleotide encoding a protein comprising a fragment of the amino add sequence of SEQ ID NO:115 having biological activity, the fragment preferably comprising eight (more preferably twenty, most preferably thirty) contiguous amino acids of SEQ ID NO:115, or a polynucleotide encoding a protein comprising a fragment of the amino acid sequence of SEQ ID NO:115 having biological activity, the fragment comprising the amino acid sequence from amino acid 35 to amino acid 44 of SEQ ID NO:115.

Other embodiments provide the gene corresponding to the cDNA sequence of SEQ ID NO:114 and SEQ ID NO:113.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:113;
      (ab) SEQ ID NO:114, but excluding the poly(A) tail at the 3′ end of SEQ ID NO:114; and
      (ac) the nucleotide sequence of the cDNA insert of clone AT340_{—}1 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:113;
      (bb) SEQ ID NO:114, but excluding the poly(A) tail at the 3′ end of SEQ ID NO:114; and
      (bc) the nucleotide sequence of the cDNA insert of clone AT340_{—}1 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:113 and SEQ ID NO:114, and extending contiguously from a nucleotide sequence corresponding to the 5′ end of SEQ ID NO:113 to a nucleotide sequence corresponding to the 3′ end of SEQ ID NO:114, but excluding the poly(A) tail at the 3′ end of SEQ ID NO:114. Also preferably the polynucleotide isolated according to the above process comprises a nucleotide sequence corresponding to the cDNA sequence of SEQ ID NO:114, and extending contiguously from a nucleotide sequence corresponding to the 5′ end of SEQ ID NO:114 to a nucleotide sequence corresponding to the 3′ end of SEQ ID NO:114, but excluding the poly(A) tail at the 3′ end of SEQ ID NO:114. Also preferably the polynucleotide isolated according to the above process comprises a nucleotide sequence corresponding to the cDNA sequence of SEQ ID NO:114 from nucleotide 19 to nucleotide 262, and extending contiguously from a nucleotide sequence corresponding to the 5′ end of said sequence of SEQ ID NO:114 from nucleotide 19 to nucleotide 262, to a nucleotide sequence corresponding to the 3′ end of said sequence of SEQ ID NO:114 from nucleotide 19 to nucleotide 262. Also preferably the polynucleotide isolated according to the above process comprises a nucleotide sequence corresponding to the cDNA sequence of SEQ ID NO:114 from nucleotide 91 to nucleotide 262, and extending contiguously from a nucleotide sequence corresponding to the 5′ end of said sequence of SEQ ID NO:114 from nucleotide 91 to nucleotide 262, to a nucleotide sequence corresponding to the 3′ end of said sequence of SEQ ID NO:114 from nucleotide 91 to nucleotide 262.

In other embodiments, the present invention provides a composition comprising a protein, wherein said protein comprises an amino acid sequence selected from the group consisting of:
(a) the amino acid sequence of SEQ ID NO:115;
(b) the amino add sequence of SEQ ID NO:115 from amino add 1 to amino acid 66;
(c) a fragment of the amino acid sequence of SEQ ID NO:115, the fragment comprising eight contiguous amino acids of SEQ ID NO:115; and
(d) the amino acid sequence encoded by the cDNA insert of clone AT340_{—}1 deposited with the ATCC under accession number 98271;
   the protein being substantially free from other mammalian proteins. Preferably such protein comprises the amino add sequence of SEQ ID NO:115 or the amino add sequence of SEQ ID NO:115 from amino acid 1 to amino add 66. In further preferred embodiments, the present invention provides a protein comprising a fragment of the amino add sequence of SEQ ID NO:115 having biological activity, the fragment preferably comprising eight (more preferably twenty, most preferably thirty) contiguous amino adds of SEQ ID NO:115, or a protein comprising a fragment of the amino add sequence of SEQ ID NO:115 having biological activity, the fragment comprising the amino acid sequence from amino acid 35 to amino add 44 of SEQ ID NO:115.

In one embodiment, the present invention provides a composition comprising an isolated polynucleotide selected from the group consisting of:
(a) a polynucleotide comprising the nucleotide sequence of SEQ ID NO:116;
(b) a polynucleotide comprising the nucleotide sequence of SEQ ID NO:116 from nucleotide 2 to nucleotide 601;
(c) a polynucleotide comprising the nucleotide sequence of SEQ ID NO:116 from nucleotide 401 to nucleotide 601;
(d) a polynucleotide comprising the nucleotide sequence of the full-length protein coding sequence of clone BG132_{—}1 deposited with the ATCC under accession number 98271;
(e) a polynucleotide encoding the full-length protein encoded by the cDNA insert of clone BG132_{—}1 deposited with the ATCC under accession number 98271;
(f) a polynucleotide comprising the nucleotide sequence of a mature protein coding sequence of clone BG132_{—}1 deposited with the ATCC under accession number 98271;
(g) a polynucleotide encoding a mature protein encoded by the cDNA insert of clone BG132_{—}1 deposited with the ATCC under accession number 98271;
(h) a polynucleotide encoding a protein comprising the amino add sequence of SEQ ID NO:17;
(i) a polynucleotide encoding a protein comprising a fragment of the amino acid sequence of SEQ ID NO:117 having biological activity, the fragment comprising eight contiguous amino acids of SEQ ID NO:117;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:116.

Preferably, such polynucleotide comprises the nucleotide sequence of SEQ ID NO:116 from nucleotide 2 to nucleotide 601; the nucleotide sequence of SEQ ID NO:116 from nucleotide 401 to nucleotide 601; the nucleotide sequence of the full-length protein coding sequence of clone BG132_{—}1 deposited with the ATCC under accession number 98271; or the nucleotide sequence of a mature protein coding sequence of clone BG132_{—}1 deposited with the ATCC under accession number 98271. In other preferred embodiments, the polynucleotide encodes the full-length or a mature protein encoded by the cDNA insert of clone BG132_{—}1 deposited with the ATCC under accession number 98271. In yet other preferred embodiments, the present invention provides a polynucleotide encoding a protein comprising the amino acid sequence of SEQ ID NO:117 from amino acid 119 to amino acid 200. In further preferred embodiments, the present invention provides a polynucleotide encoding a protein comprising a fragment of the amino acid sequence of SEQ ID NO:117 having biological activity, the fragment preferably comprising eight (more preferably twenty, most preferably thirty) contiguous amino acids of SEQ ID NO:117, or a polynucleotide encoding a protein comprising a fragment of the amino acid sequence of SEQ ID NO:117 having biological activity, the fragment comprising the amino acid sequence from amino acid 95 to amino acid 104 of SEQ ID NO:117.

Other embodiments provide the gene corresponding to the cDNA sequence of SEQ ID NO:116 and SEQ ID NO:118.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:116;
      (ab) SEQ ID NO:118, but excluding the poly(A) tail at the 3′ end of SEQ ID NO:118; and
      (ac) the nucleotide sequence of the cDNA insert of clone BG132_{—}1 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:116;
      (bb) SEQ ID NO:118, but excluding the poly(A) tail at the 3′ end of SEQ ID NO:118; and
      (bc) the nucleotide sequence of the cDNA insert of clone BG132_{—}1 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:116 and SEQ ID NO:118, and extending contiguously from a nucleotide sequence corresponding to the 5′ end of SEQ ID NO:116 to a nucleotide sequence corresponding to the 3′ end of SEQ ID NO:118, but excluding the poly(A) tail at the 3′ end of SEQ ID NO:118. Also preferably the polynucleotide isolated according to the above process comprises a nucleotide sequence corresponding to the cDNA sequence of SEQ ID NO:116, and extending contiguously from a nucleotide sequence corresponding to the 5′ end of SEQ ID NO:116 to a nucleotide sequence corresponding to the 3′ end of SEQ ID NO:116. Also preferably the polynucleotide isolated according to the above process comprises a nucleotide sequence corresponding to the cDNA sequence of SEQ ID NO:116 from nucleotide 2 to nucleotide 601, and extending contiguously from a nucleotide sequence corresponding to the 5′ end of said sequence of SEQ ID NO:116 from nucleotide 2 to nucleotide 601, to a nucleotide sequence corresponding to the 3′ end of said sequence of SEQ ID NO:116 from nucleotide 2 to nucleotide 601. Also preferably the polynucleotide isolated according to the above process comprises a nucleotide sequence corresponding to the cDNA sequence of SEQ ID NO:116 from nucleotide 401 to nucleotide 601, and extending contiguously from a nucleotide sequence corresponding to the 5′ end of said sequence of SEQ ID NO:116 from nucleotide 401 to nucleotide 601, to a nucleotide sequence corresponding to the 3′ end of said sequence of SEQ ID NO:116 from nucleotide 401 to nucleotide 601.

In other embodiments, the present invention provides a composition comprising a protein, wherein said protein comprises an amino acid sequence selected from the group consisting of:
(a) the amino add sequence of SEQ ID NO:117;
(b) the amino acid sequence of SEQ ID NO:117 from amino acid 119 to amino acid 200;
(c) a fragment of the amino acid sequence of SEQ ID NO:117, the fragment comprising eight contiguous amino acids of SEQ ID NO:117; and
(d) the amino acid sequence encoded by the cDNA insert of clone BG132 μl deposited with the ATCC under accession number 98271;
   the protein being substantially free from other mammalian proteins. Preferably such protein comprises the amino acid sequence of SEQ ID NO:117 or the amino acid sequence of SEQ ID NO:117 from amino acid 119 to amino acid 200. In further preferred embodiments, the present invention provides a protein comprising a fragment of the amino acid sequence of SEQ ID NO:117 having biological activity, the fragment preferably comprising eight (more preferably twenty, most preferably thirty) contiguous amino acids of SEQ ID NO:117, or a protein comprising a fragment of the amino acid sequence of SEQ ID NO:117 having biological activity, the fragment comprising the amino acid sequence from amino acid 95 to amino add 104 of SEQ ID NO:117.

In one embodiment, the present invention provides a composition comprising an isolated polynucleotide selected from the group consisting of:
(a) a polynucleotide comprising the nucleotide sequence of SEQ ID NO:119;
(b) a polynucleotide comprising the nucleotide sequence of SEQ ID NO:119 from nucleotide 225 to nucleotide 701;
(c) a polynucleotide comprising the nucleotide sequence of the full-length protein coding sequence of clone BG219_{—}2 deposited with the ATCC under accession number 98271;
(d) a polynucleotide encoding the full-length protein encoded by the cDNA insert of clone BG219_{—}2 deposited with the ATCC under accession number 98271;
(e) a polynucleotide comprising the nucleotide sequence of a mature protein coding sequence of clone BG219_{—}2 deposited with the ATCC under accession number 98271;
(f) a polynucleotide encoding a mature protein encoded by the cDNA insert of clone BG219_{—}2 deposited with the ATCC under accession number 98271;
(g) a polynucleotide encoding a protein comprising the amino add sequence of SEQ ID NO:120;
(h) a polynucleotide encoding a protein comprising a fragment of the amino add sequence of SEQ ID NO:120 having biological activity, the fragment comprising eight contiguous amino adds of SEQ ID NO:120;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:119.

Preferably, such polynucleotide comprises the nucleotide sequence of SEQ ID NO:119 from nucleotide 225 to nucleotide 701; the nucleotide sequence of the full-length protein coding sequence of clone BG219_{—}2 deposited with the ATCC under accession number 98271; or the nucleotide sequence of a mature protein coding sequence of clone BG219_{—}2 deposited with the ATCC under accession number 98271. In other preferred embodiments, the polynucleotide encodes the full-length or a mature protein encoded by the cDNA insert of clone BG219_{—}2 deposited with the ATCC under accession number 98271. In yet other preferred embodiments, the present invention provides a polynucleotide encoding a protein comprising the amino acid sequence of SEQ ID NO:120 from amino acid 1 to amino acid 97. In further preferred embodiments, the present invention provides a polynucleotide encoding a protein comprising a fragment of the amino acid sequence of SEQ ID NO:120 having biological activity, the fragment preferably comprising eight (more preferably twenty, most preferably thirty) contiguous amino adds of SEQ ID NO:120, or a polynucleotide encoding a protein comprising a fragment of the amino acid sequence of SEQ ID NO:120 having biological activity, the fragment comprising the amino acid sequence from amino add 74 to amino acid 83 of SEQ ID NO:120.

Other embodiments provide the gene corresponding to the cDNA sequence of SEQ ID NO:119.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:119, but excluding the poly(A) tail at the 3′ end of SEQ ID NO:119; and
      (ab) the nucleotide sequence of the cDNA insert of clone BG219_{—}2 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:119, but excluding the poly(A) tail at the 3′ end of SEQ ID NO:119; and
      (bb) the nucleotide sequence of the cDNA insert of clone BG219_{—}2 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:119, and extending contiguously from a nucleotide sequence corresponding to the 5′ end of SEQ ID NO:119 to a nucleotide sequence corresponding to the 3′ end of SEQ ID NO:119, but excluding the poly(A) tail at the 3′ end of SEQ ID NO:119. Also preferably the polynucleotide isolated according to the above process comprises a nucleotide sequence corresponding to the cDNA sequence of SEQ ID NO:119 from nucleotide 225 to nucleotide 701, and extending contiguously from a nucleotide sequence corresponding to the 5′ end of said sequence of SEQ ID NO:119 from nucleotide 225 to nucleotide 701, to a nucleotide sequence corresponding to the 3′ end of said sequence of SEQ ID NO:119 from nucleotide 225 to nucleotide 701.

In other embodiments, the present invention provides a composition comprising a protein, wherein said protein comprises an amino acid sequence selected from the group consisting of:
(a) the amino acid sequence of SEQ ID NO:120;
(b) the amino acid sequence of SEQ ID NO:120 from amino acid 1 to amino acid 97;
(c) a fragment of the amino acid sequence of SEQ ID NO:120, the fragment comprising eight contiguous amino acids of SEQ ID NO:120; and
(d) the amino acid sequence encoded by the cDNA insert of clone BG219_{—}2 deposited with the ATCC under accession number 98271;
   the protein being substantially free from other mammalian proteins. Preferably such protein comprises the amino add sequence of SEQ ID NO:120 or the amino acid sequence of SEQ ID NO:120 from amino acid 1 to amino acid 97. In further preferred embodiments, the present invention provides a protein comprising a fragment of the amino acid sequence of SEQ ID NO:120 having biological activity, the fragment preferably comprising eight (more preferably twenty, most preferably thirty) contiguous amino acids of SEQ ID NO:120, or a protein comprising a fragment of the amino acid sequence of SEQ ID NO:120 having biological activity, the fragment comprising the amino acid sequence from amino acid 74 to amino acid 83 of SEQ ID NO:120.

In one embodiment, the present invention provides a composition comprising an isolated polynucleotide selected from the group consisting of:
(a) a polynucleotide comprising the nucleotide sequence of SEQ ID NO:121;
(b) a polynucleotide comprising the nucleotide sequence of SEQ ID NO:121 from nucleotide 2115 to nucleotide 2510;
(c) a polynucleotide comprising the nucleotide sequence of SEQ ID NO:121 from nucleotide 1 to nucleotide 324;
(d) a polynucleotide comprising the nucleotide sequence of the full-length protein coding sequence of clone BG366_{—}2 deposited with the ATCC under accession number 98271;
(e) a polynucleotide encoding the full-length protein encoded by the cDNA insert of clone BG366_{—}2 deposited with the ATCC under accession number 98271;
(f) a polynucleotide comprising the nucleotide sequence of a mature protein coding sequence of clone BG366_{—}2 deposited with the ATCC under accession number 98271;
(g) a polynucleotide encoding a mature protein encoded by the cDNA insert of clone BG366_{—}2 deposited with the ATCC under accession number 98271; (h) a polynucleotide encoding a protein comprising the amino acid sequence of SEQ ID NO:122;
(i) a polynucleotide encoding a protein comprising a fragment of the amino acid sequence of SEQ ID NO:122 having biological activity, the fragment comprising eight contiguous amino acids of SEQ ID NO:122;
10) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:121.

Preferably, such polynucleotide comprises the nucleotide sequence of SEQ ID NO:121 from nucleotide 2115 to nucleotide 2510; the nucleotide sequence of SEQ ID NO:121 from nucleotide 1 to nucleotide 324; the nucleotide sequence of the full-length protein coding sequence of clone BG366_{—}2 deposited with the ATCC under accession number 98271; or the nucleotide sequence of a mature protein coding sequence of clone BG366_{—}2 deposited with the ATCC under accession number 98271. In other preferred embodiments, the polynucleotide encodes the full-length or a mature protein encoded by the cDNA insert of clone BG366_{—}2 deposited with the ATCC under accession number 98271. In further preferred embodiments, the present invention provides a polynucleotide encoding a protein comprising a fragment of the amino acid sequence of SEQ ID NO:122 having biological activity, the fragment preferably comprising eight (more preferably twenty, most preferably thirty) contiguous amino acids of SEQ ID NO:122, or a polynucleotide encoding a protein comprising a fragment of the amino acid sequence of SEQ ID NO:122 having biological activity, the fragment comprising the amino acid sequence from amino acid 61 to amino acid 70 of SEQ ID NO:122.

Other embodiments provide the gene corresponding to the cDNA sequence of SEQ ID NO:121.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:121, but excluding the poly(A) tail at the 3′ end of SEQ ID NO:121; and
      (ab) the nucleotide sequence of the cDNA insert of clone BG366_{—}2 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:121, but excluding the poly(A) tail at the 3′ end of SEQ ID NO:121; and
      (bb) the nucleotide sequence of the cDNA insert of clone BG366_{—}2 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:121, and extending contiguously from a nucleotide sequence corresponding to the 5′ end of SEQ ID NO:121 to a nucleotide sequence corresponding to the 3′ end of SEQ ID NO:121, but excluding the poly(A) tail at the 3′ end of SEQ ID NO:121. Also preferably the polynucleotide isolated according to the above process comprises a nucleotide sequence corresponding to the cDNA sequence of SEQ ID NO:121 from nucleotide 2115 to nucleotide 2510, and extending contiguously from a nucleotide sequence corresponding to the 5′ end of said sequence of SEQ ID NO:121 from nucleotide 2115 to nucleotide 2510, to a nucleotide sequence corresponding to the 3′ end of said sequence of SEQ ID NO:121 from nucleotide 2115 to nucleotide 2510. Also preferably the polynucleotide isolated according to the above process comprises a nucleotide sequence corresponding to the cDNA sequence of SEQ ID NO:121 from nucleotide 1 to nucleotide 324, and extending contiguously from a nucleotide sequence corresponding to the 5′ end of said sequence of SEQ ID NO:121 from nucleotide 1 to nucleotide 324, to a nucleotide sequence corresponding to the 3′ end of said sequence of SEQ ID NO:121 from nucleotide 1 to nucleotide 324.

In other embodiments, the present invention provides a composition comprising a protein, wherein said protein comprises an amino acid sequence selected from the group consisting of:
(a) the amino acid sequence of SEQ ID NO:122;
(b) a fragment of the amino acid sequence of SEQ ID NO:122, the fragment comprising eight contiguous amino acids of SEQ ID NO:122; and
(c) the amino acid sequence encoded by the cDNA insert of clone BG366_{—}2 deposited with the ATCC under accession number 98271;
   the protein being substantially free from other mammalian proteins. Preferably such protein comprises the amino acid sequence of SEQ ID NO:122. In further preferred embodiments, the present invention provides a protein comprising a fragment of the amino acid sequence of SEQ ID NO:122 having biological activity, the fragment preferably comprising eight (more preferably twenty, most preferably thirty) contiguous amino acids of SEQ ID NO:122, or a protein comprising a fragment of the amino acid sequence of SEQ ID NO:122 having biological activity, the fragment comprising the amino acid sequence from amino acid 61 to amino acid 70 of SEQ ID NO:122.

In one embodiment, the present invention provides a composition comprising an isolated polynucleotide selected from the group consisting of:
(a) a polynucleotide comprising the nucleotide sequence of SEQ ID NO:123;
(b) a polynucleotide comprising the nucleotide sequence of SEQ ID NO:123 from nucleotide 27 to nucleotide 215;
(c) a polynucleotide comprising the nucleotide sequence of SEQ ID NO:123 from nucleotide 2-7 to nucleotide 181;
(d) a polynucleotide comprising the nucleotide sequence of the full-length protein coding sequence of clone BV172_{—}2 deposited with the ATCC under accession number 98271;
(e) a polynucleotide encoding the full-length protein encoded by the cDNA insert of clone BV172_{—}2 deposited with the ATCC under accession number 98271;
(f) a polynucleotide comprising the nucleotide sequence of a mature protein coding sequence of clone BV172_{—}2 deposited with the ATCC under accession number 98271;
(g) a polynucleotide encoding a mature protein encoded by the cDNA insert of clone BV172_{—}2 deposited with the ATCC under accession number 98271;
(h) a polynucleotide encoding a protein comprising the amino acid sequence of SEQ ID NO:124;
(i) a polynucleotide encoding a protein comprising a fragment of the amino acid sequence of SEQ ID NO:124 having biological activity, the fragment comprising eight contiguous amino acids of SEQ ID NO:124;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:123.

Preferably, such polynucleotide comprises the nucleotide sequence of SEQ ID NO:123 from nucleotide 27 to nucleotide 215; the nucleotide sequence of SEQ ID NO:123 from nucleotide 27 to nucleotide 181; the nucleotide sequence of the full-length protein coding sequence of clone BV172_{—}2 deposited with the ATCC under accession number 98271; or the nucleotide sequence of a mature protein coding sequence of clone BV172_{—}2 deposited with the ATCC under accession number 98271. In other preferred embodiments, the polynucleotide encodes the full-length or a mature protein encoded by the cDNA insert of clone BV172_{—}2 deposited with the ATCC under accession number 98271. In yet other preferred embodiments, the present invention provides a polynucleotide encoding a protein comprising the amino acid sequence of SEQ ID NO:124 from amino acid 1 to amino acid 51. In further preferred embodiments, the present invention provides a polynucleotide encoding a protein comprising a fragment of the amino acid sequence of SEQ ID NO:124 having biological activity, the fragment preferably comprising eight (more preferably twenty, most preferably thirty) contiguous amino adds of SEQ ID NO:124, or a polynucleotide encoding a protein comprising a fragment of the amino acid sequence of SEQ ID NO:124 having biological activity, the fragment comprising the amino acid sequence from amino acid 26 to amino acid 35 of SEQ ID NO:124.

Other embodiments provide the gene corresponding to the cDNA sequence of SEQ ID NO:123.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:123, but excluding the poly(A) tail at the 3′ end of SEQ ID NO:123; and
      (ab) the nucleotide sequence of the cDNA insert of clone BV172_{—}2 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of
      (ba) SEQ ID NO:123, but excluding the poly(A) tail at the 3′ end of SEQ ID NO:123; and
      (bb) the nucleotide sequence of the cDNA insert of clone BV172_{—}2 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:123, and extending contiguously from a nucleotide sequence corresponding to the 5′ end of SEQ ID NO:123 to a nucleotide sequence corresponding to the 3′ end of SEQ ID NO:123, but excluding the poly(A) tail at the 3′ end of SEQ ID NO:123. Also preferably the polynucleotide isolated according to the above process comprises a nucleotide sequence corresponding to the cDNA sequence of SEQ ID NO:123 from nucleotide 27 to nucleotide 215, and extending contiguously from a nucleotide sequence corresponding to the 5′ end of said sequence of SEQ ID NO:123 from nucleotide 27 to nucleotide 215, to a nucleotide sequence corresponding to the 3′ end of said sequence of SEQ ID NO:123 from nucleotide 27 to nucleotide 215. Also preferably the polynucleotide isolated according to the above process comprises a nucleotide sequence corresponding to the cDNA sequence of SEQ ID NO:123 from nucleotide 27 to nucleotide 181, and extending contiguously from a nucleotide sequence corresponding to the 5′ end of said sequence of SEQ ID NO:123 from nucleotide 27 to nucleotide 181, to a nucleotide sequence corresponding to the 3′ end of said sequence of SEQ ID NO:123 from nucleotide 27 to nucleotide 181.

In other embodiments, the present invention provides a composition comprising a protein, wherein said protein comprises an amino add sequence selected from the group consisting of:
(a) the amino acid sequence of SEQ ID NO:124;
(b) the amino add sequence of SEQ ID NO:124 from amino add 1 to amino acid 51;
(c) a fragment of the amino acid sequence of SEQ ID NO:124, the fragment comprising eight contiguous amino acids of SEQ ID NO:124; and
(d) the amino add sequence encoded by the cDNA insert of clone BV172_{—}2 deposited with the ATCC under accession number 98271; the protein being substantially free from other mammalian proteins. Preferably such protein comprises the amino acid sequence of SEQ ID NO:124 or the amino add sequence of SEQ ID NO:124 from amino acid 1 to amino acid 51. In further preferred embodiments, the present invention provides a protein comprising a fragment of the amino add sequence of SEQ ID NO:124 having biological activity, the fragment preferably comprising eight (more preferably twenty, most preferably thirty) contiguous amino adds of SEQ ID NO:124, or a protein comprising a fragment of the amino add sequence of SEQ ID NO:124 having biological activity, the fragment comprising the amino add sequence from amino add 26 to amino acid 35 of SEQ ID NO:124.

In one embodiment, the present invention provides a composition comprising an isolated polynucleotide selected from the group consisting of:
(a) a polynucleotide comprising the nucleotide sequence of SEQ ID NO:125;
(b) a polynucleotide comprising the nucleotide sequence of SEQ ID NO:125 from nucleotide 338 to nucleotide 409;
(c) a polynucleotide comprising the nucleotide sequence of SEQ ID NO:125 from nucleotide 362 to nucleotide 409;
(d) a polynucleotide comprising the nucleotide sequence of SEQ ID NO:125 from nucleotide 270 to nucleotide 419;
(e) a polynucleotide comprising the nucleotide sequence of the full-length protein coding sequence of clone CC247_{—}10 deposited with the ATCC under accession number 98271;
(f) a polynucleotide encoding the full-length protein encoded by the cDNA insert of clone CC24710 deposited with the ATCC under accession number 98271;
(g) a polynucleotide comprising the nucleotide sequence of a mature protein coding sequence of clone CC2470 deposited with the ATCC under accession number 98271;
(h) a polynucleotide encoding a mature protein encoded by the cDNA insert of clone CC247_{—}10 deposited with the ATCC under accession number 9821;
(i) a polynucleotide encoding a protein comprising the amino acid sequence of SEQ ID NO:126;
(j) a polynucleotide encoding a protein comprising a fragment of the amino acid sequence of SEQ ID NO:126 having biological activity, the fragment comprising eight contiguous amino acids of SEQ ID NO:126;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:125.

Preferably, such polynucleotide comprises the nucleotide sequence of SEQ ID NO:125 from nucleotide 338 to nucleotide 409; the nucleotide sequence of SEQ ID NO:125 from nucleotide 362 to nucleotide 409; the nucleotide sequence of SEQ ID NO:125 from nucleotide 270 to nucleotide 419; the nucleotide sequence of the full-length protein coding sequence of clone CC247_{—}10 deposited with the ATCC under accession number 98271; or the nucleotide sequence of a mature protein coding sequence of clone CC247_{—}10 deposited with the ATCC under accession number 98271. In other preferred embodiments, the polynucleotide encodes the full-length or a mature protein encoded by the cDNA insert of clone CC247_{—}10 deposited with the ATCC under accession number 98271. In further preferred embodiments, the present invention provides a polynucleotide encoding a protein comprising a fragment of the amino acid sequence of SEQ ID NO:126 having biological activity, the fragment preferably comprising eight. (more preferably twenty, most preferably thirty) contiguous amino adds of SEQ ID NO:126, or a polynucleotide encoding a protein comprising a fragment of the amino add sequence of SEQ ID NO:126 having biological activity, the fragment comprising the amino add sequence from amino acid 7 to amino add 16 of SEQ ID NO:126.

Other embodiments provide the gene corresponding to the cDNA sequence of SEQ ID NO:125.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C to a nucleotide sequence selected from the group consisting of:
      (aa) SEQ ID NO:125, but excluding the poly(A) tail at the 3′ end of SEQ ID NO:125; and
      (ab) the nucleotide sequence of the DNA insert of clone CC247_{—}10 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C to a nucleotide sequence selected from the group consisting of:
      (ba) SEQ ID NO:125, but excluding the poly(A) tail at the 3′ end of SEQ ID NO:125; and
      (bb) the nucleotide sequence of the cDNA insert of clone CC247_{—}10 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:125, and extending contiguously from a nucleotide sequence corresponding to the 5′ end of SEQ ID NO:125 to a nucleotide sequence corresponding to the 3′ end of SEQ ID NO:125, but excluding the poly(A) tail at the 3′ end of SEQ ID NO:125. Also preferably the polynucleotide isolated according to the above process comprises a nucleotide sequence corresponding to the cDNA sequence of SEQ ID NO:125 from nucleotide 338 to nucleotide 409, and extending contiguously from a nucleotide sequence corresponding to the 5′ end of said sequence of SEQ ID NO:125 from nucleotide 338 to nucleotide 409, to a nucleotide sequence corresponding to the 3′ end of said sequence of SEQ ID NO:125 from nucleotide 338 to nucleotide 409. Also preferably the polynucleotide isolated according to the above process comprises a nucleotide sequence corresponding to the cDNA sequence of SEQ ID NO:125 from nucleotide 362 to nucleotide 409, and extending contiguously from a nucleotide sequence corresponding to the 5′ end of said sequence of SEQ ID NO:125 from nucleotide 362 to nucleotide 409, to a nucleotide sequence corresponding to the 3′ end of said sequence of SEQ ID NO:125 from nucleotide 362 to nucleotide 409. Also preferably the polynucleotide isolated according to the above process comprises a nucleotide sequence corresponding to the cDNA sequence of SEQ ID NO:125 from nucleotide 270 to nucleotide 419, and extending contiguously from a nucleotide sequence corresponding to the 5′ end of said sequence of SEQ ID NO:125 from nucleotide 270 to nucleotide 419, to a nucleotide sequence corresponding to the 3′ end of said sequence of SEQ ID NO:125 from nucleotide 270 to nucleotide 419.

In other embodiments, the present invention provides a composition comprising a protein, wherein said protein comprises an amino add sequence selected from the group consisting of:
(a) the amino acid sequence of SEQ ID NO:126;
(b) a fragment of the amino acid sequence of SEQ ID NO:126, the fragment comprising eight contiguous amino acids of SEQ ID NO:126; and
(c) the amino add sequence encoded by the cDNA inset of clone CC247_{—}10 deposited with the ATCC under accession number 98271; the protein being substantially free from other mammalian proteins. Preferably such protein comprises the amino add sequence of SEQ ID NO:126. In further preferred embodiments, the present invention provides a protein comprising a fragment of the amino acid sequence of SEQ ID NO:126 having biological activity, the fragment preferably comprising eight (more preferably twenty, most preferably thirty) contiguous amino acids of SEQ ID NO:126, or a protein comprising a fragment of the amino acid sequence of SEQ ID NO:126 having biological activity, the fragment comprising the amino acid sequence from amino acid 7 to amino add 16 of SEQ ID NO:126.

In one embodiment, the present invention provides a composition comprising an isolated polynucleotide selected from the group consisting of:
(a) a polynucleotide comprising the nucleotide sequence of SEQ ID NO:127;
(b) a polynucleotide comprising the nucleotide sequence of SEQ ID NO:127 from nucleotide 128 to nucleotide 1600;
(c) a polynucleotide comprising the nucleotide sequence of SEQ ID NO:127 from nucleotide 281 to nucleotide 1600;
(d) a polynucleotide comprising the nucleotide sequence of SEQ ID NO:127 from nucleotide 62 to nucleotide 373;
(e) a polynucleotide comprising the nucleotide sequence of the full-length protein coding sequence of clone CI480_{—}9 deposited with the ATCC under accession number 98271;
(f) a polynucleotide encoding the full-length protein encoded by the cDNA insert of clone CI480_{—}9 deposited with the ATCC under accession number 98271;
(g) a polynucleotide comprising the nucleotide sequence of a mature protein coding sequence of clone CI480_{—}9 deposited with the ATCC under accession number 98271;
(h) a polynucleotide encoding a mature protein encoded by the cDNA insert of clone CI480_{—}9 deposited with the ATCC under accession number 98271;
(i) a polynucleotide encoding a protein comprising the amino acid sequence of SEQ ID NO:128;
(j) a polynucleotide encoding a protein comprising a fragment of the amino acid sequence of SEQ ID NO:128 having biological activity, the fragment comprising eight contiguous amino acids of SEQ ID NO:128;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:127.

Preferably, such polynucleotide comprises the nucleotide sequence of SEQ ID NO:127 from nucleotide 128 to nucleotide 1600; the nucleotide sequence of SEQ ID NO:127 from nucleotide 281 to nucleotide 1600; the nucleotide sequence of SEQ ID NO:127 from nucleotide 62 to nucleotide 373; the nucleotide sequence of the full-length protein coding sequence of clone CI480_{—}9 deposited with the ATCC under accession number 98271; or the nucleotide sequence of a mature protein coding sequence of clone CI480_{—}9 deposited with the ATCC under accession number 981. In other preferred embodiments, the polynucleotide encodes the full-length or a mature protein encoded by the cDNA insert of clone CI480_{—}9 deposited with the ATCC under accession number 98271. In yet other preferred embodiments, the present invention provides a polynucleotide encoding a protein comprising the amino add sequence of SEQ ID NO:128 from amino acid 1 to amino add 82. In further preferred embodiments, the present invention provides a polynucleotide encoding a protein comprising a fragment of the amino acid sequence of SEQ ID NO:128 having biological activity, the fragment preferably comprising eight (more preferably twenty, most preferably) contiguous amino acids of SEQ ID NO:128, or a polynucleotide encoding a protein comprising a fragment of the amino acid sequence of SEQ ID NO:128 having biological activity, the fragment comprising the amino acid sequence from amino acid 240 to amino acid 249 of SEQ ID NO:128.

Other embodiments provide the gene corresponding to the cDNA sequence of SEQ ID NO:127.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:127, but excluding the poly(A) tail at the 3′ end of SEQ ID NO:127; and
      (ab) the nucleotide sequence of the cDNA insert of clone CI480_{—}9 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:127, but excluding the poly(A) tail at the 3′ end of SEQ ID NO:127; and
      (bb) the nucleotide sequence of the cDNA insert of clone CI480_{—}9 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:127, and extending contiguously from a nucleotide sequence corresponding to the 5′ end of SEQ ID NO:127 to a nucleotide sequence corresponding to the 3′ end of SEQ ID NO:127, but excluding the poly(A) tail at the 3′ end of SEQ ID NO:127. Also preferably the polynucleotide isolated according to the above process comprises a nucleotide sequence corresponding to the cDNA sequence of SEQ ID NO:127 from nucleotide 128 to nucleotide 1600, and extending contiguously from a nucleotide sequence corresponding to the 5′ end of said sequence of SEQ ID NO:127 from nucleotide 128 to nucleotide 1600, to a nucleotide sequence corresponding to the 3′ end of said sequence of SEQ ID NO:127 from nucleotide 128 to nucleotide 1600. Also preferably the polynucleotide isolated according to the above process comprises a nucleotide sequence corresponding to the cDNA sequence of SEQ ID NO:127 from nucleotide 281 to nucleotide 1600, and extending contiguously from a nucleotide sequence corresponding to the 5′ end of said sequence of SEQ ID NO:127 from nucleotide 281 to nucleotide 1600, to a nucleotide sequence corresponding to the 3′ end of said sequence of SEQ ID NO:127 from nucleotide 281 to nucleotide 1600. Also preferably the polynucleotide isolated according to the above process comprises a nucleotide sequence corresponding to the cDNA sequence of SEQ ID NO:127 from nucleotide 62 to nucleotide 373, and extending contiguously from a nucleotide sequence corresponding to the 5′ end of said sequence of SEQ ID NO:127 from nucleotide 62 to nucleotide 373, to a nucleotide sequence corresponding to the 3′ end of said sequence of SEQ ID NO:127 from nucleotide 62 to nucleotide 373.

In other embodiments, the present invention provides a composition comprising a protein, wherein said protein comprises an amino acid sequence selected from the group consisting of:
(a) the amino acid sequence of SEQ ID NO:128;
(b) the amino acid sequence of SEQ ID NO:128 from amino add 1 to amino add 82;
(c) a fragment of the amino acid sequence of SEQ ID NO:128, the fragment comprising eight contiguous amino adds of SEQ ID NO:128; and
(d) the amino acid sequence encoded by the cDNA insert of clone CI480_{—}9 deposited with the ATCC under accession number 98271;
   the protein being substantially free from other mammalian proteins. Preferably such protein comprises the amino acid sequence of SEQ ID NO:128 or the amino acid sequence of SEQ ID NO:128 from amino acid 1 to amino acid 82. In further preferred embodiments, the present invention provides a protein comprising a fragment of the amino acid sequence of SEQ ID NO:128 having biological activity, the fragment preferably comprising eight (more preferably twenty, most preferably thirty) contiguous amino acids of SEQ ID NO:128, or a protein comprising a fragment of the amino acid sequence of SEQ ID NO:128 having biological activity, the fragment comprising the amino add sequence from amino acid 240 to amino acid 249 of SEQ ID NO:128.

In one embodiment, the present invention provides a composition comprising an isolated polynucleotide selected from the group consisting of:
(a) a polynucleotide comprising the nucleotide sequence of SEQ ID NO:129;
(b) a polynucleotide comprising the nucleotide sequence of SEQ ID NO:129 from nucleotide 383 to nucleotide 3958;
(c) a polynucleotide comprising the nucleotide sequence of SEQ ID NO:129 from nucleotide 470 to nucleotide 3958;
(d) a polynucleotide comprising the nucleotide sequence of SEQ ID NO:129 from nucleotide 271 to nucleotide 488;
(e) a polynucleotide comprising the nucleotide sequence of the full-length protein coding sequence of clone CO722_{—}1 deposited with the ATCC under accession number 98271;
(f) a polynucleotide encoding the full-length protein encoded by the cDNA insert of clone CO722_{—}1 deposited with the ATCC under accession number 98271;
(g) a polynucleotide comprising the nucleotide sequence of a mature protein coding sequence of clone CO722_{—}1 deposited with the ATCC under accession number 98271;
(h) a polynucleotide encoding a mature protein encoded by the cDNA insert of clone CO722_{—}1 deposited with the ATCC under accession number 98271;
(i) a polynucleotide encoding a protein comprising the amino acid sequence of SEQ ID NO:130;
(j) a polynucleotide encoding a protein comprising a fragment of the amino acid sequence of SEQ ID NO:130 having biological activity, the fragment comprising eight contiguous amino adds of SEQ ID NO:130;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:129.

Preferably, such polynucleotide comprises the nucleotide sequence of SEQ ID NO:129 from nucleotide 383 to nucleotide 3958; the nucleotide sequence of SEQ ID NO:129 from nucleotide 470 to nucleotide 3958; the nucleotide sequence of SEQ ID NO:129 from nucleotide 271 to nucleotide 488; the nucleotide sequence of the full-length protein coding sequence of clone CO722_{—}1 deposited with the ATCC under accession number 98271; or the nucleotide sequence of a mature protein coding sequence of clone CO722_{—}1 deposited with the ATCC under accession number 98271. In other preferred embodiments, the polynucleotide encodes the full or a mature protein encoded by the cDNA insert of clone CO722_{—}1 deposited with the ATCC under accession number 98271. In yet other preferred embodiments, the present invention provides a polynucleotide encoding a protein comp the amino add sequence of SEQ ID NO:130 from amino add 1 to amino acid 34. In further preferred embodiments, the present invention provides a polynucleotide encoding a protein comprising a fragment of the amino acid sequence of SEQ ID NO:130 having biological activity, the fragment preferably comprising eight (more preferably twenty, most preferably thirty) contiguous amino acids of SEQ ID NO:130, or a polynucleotide encoding a protein comprising a fragment of the amino add sequence of SEQ ID NO:130 having biological activity, the fragment comprising the amino add sequence from amino acid 591 to amino add 600 of SEQ ID NO:130.

Other embodiments provide the gene corresponding to the cDNA sequence of SEQ ID NO:129.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:129, but excluding the poly(A) tail at the 3′ end of SEQ ID NO:129; and
   (ab) the nucleotide sequence of the cDNA insert of clone CO722_{—}1 deposited with the ATCC under accession number 98271;
      (ii) hybridizing said probe(s) to human genomic DNA in conditions at least as stringent as 4×SSC at 50 degrees C.; and

(iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:129, but excluding the poly(A) tail at the 3′ end of SEQ ID NO:129; and
      (bb) the nucleotide sequence of the cDNA insert of clone CO722_{—}1 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:129, and extending contiguously from a nucleotide sequence corresponding to the 5′ end of SEQ ID NO:129 to a nucleotide sequence corresponding to the 3′ end of SEQ ID NO:129, but excluding the poly(A) tail at the 3′ end of SEQ ID NO:129. Also preferably the polynucleotide isolated according to the above process comprises a nucleotide sequence corresponding to the cDNA sequence of SEQ ID NO:129 from nucleotide 383 to nucleotide 3958, and extending contiguously from a nucleotide sequence corresponding to the 5′ end of said sequence of SEQ ID NO:129 from nucleotide 383 to nucleotide 3958, to a nucleotide sequence corresponding to the 3′ end of said sequence of SEQ ID NO:129 from nucleotide 383 to nucleotide 3958. Also preferably the polynucleotide isolated according to the above process comprises a nucleotide sequence corresponding to the cDNA sequence of SEQ ID NO:129 from nucleotide 470 to nucleotide 3958, and extending contiguously from a nucleotide sequence corresponding to the 5′ end of said sequence of SEQ ID NO:129 from nucleotide 470 to nucleotide 3958, to a nucleotide sequence corresponding to the 3′ end of said sequence of SEQ ID NO:129 from nucleotide 470 to nucleotide 3958. Also preferably the polynucleotide isolated according to the above process comprises a nucleotide sequence corresponding to the cDNA sequence of SEQ ID NO:129 from nucleotide 271 to nucleotide 488, and extending contiguously from a nucleotide sequence corresponding to the 5′ end of said sequence of SEQ ID NO:129 from nucleotide 271 to nucleotide 488, to a nucleotide sequence corresponding to the 3′ end of said sequence of SEQ ID NO:129 from nucleotide 271 to nucleotide 488.

In other embodiments, the present invention provides a composition comprising a protein, wherein said protein comprises an amino add sequence selected from the group consisting of:
(a) the amino acid sequence of SEQ ID NO:130;
(b) the amino add sequence of SEQ ID NO:130 from amino add 1 to amino acid 34;
(c) a fragment of the amino add sequence of SEQ ID NO:130, the fragment comprising eight contiguous amino adds of SEQ ID NO:130; and
(d) the amino add sequence encoded by the cDNA insert of clone CO7221 deposited with the ATCC under accession number 98271;
   the protein being substantially free from other mammalian proteins. Preferably such protein comprises the amino add sequence of SEQ ID NO:130 or the amino add sequence of SEQ ID NO:130 from amino add 1 to amino acid 34. In further preferred embodiments, the present invention provides a protein comprising a fragment of the amino acid sequence of SEQ ID NO:130 having biological activity, the fragment preferably comprising eight (more preferably twenty, most preferably thirty) contiguous amino acids of SEQ ID NO:130, or a protein comprising a fragment of the amino acid sequence of SEQ ID NO:130 having biological activity, the fragment comprising the amino add sequence from amino acid 591 to amino acid 600 of SEQ ID NO:130.

In one embodiment, the present invention provides a composition comprising an isolated polynucleotide selected from the group consisting of:
(a) a polynucleotide comprising the nucleotide sequence of SEQ ID NO:131;
(b) a polynucleotide comprising the nucleotide sequence of SEQ ID NO:131 from nucleotide 914 to nucleotide 2353;
(c) a polynucleotide comprising the nucleotide sequence of SEQ ID NO:131 from nucleotide 1793 to nucleotide 2353;
(d) a polynucleotide comprising the nucleotide sequence of SEQ ID NO:131 from nucleotide 1037 to nucleotide 1260;
(e) a polynucleotide comprising the nucleotide sequence of the full-length protein coding sequence of clone CI748_{—}2 deposited with the ATCC under accession number 98271;
(f) a polynucleotide encoding the full-length protein encoded by the cDNA insert of clone CI748_{—}2 deposited with the ATCC under accession number 98271;
(g) a polynucleotide comprising the nucleotide sequence of a mature protein coding sequence of clone C1748_{—}2 deposited with the ATCC under accession number 98271;
(h) a polynucleotide encoding a mature protein encoded by the cDNA insert of clone C748_{—}2 deposited with the ATCC under a on number 98271;
(i) a polynucleotide encoding a protein comprising the amino acid sequence of SEQ ID NO:132;
(j) a polynucleotide encoding a protein comprising a fragment of the amino acid sequence of SEQ ID NO:132 having biological activity, the fragment comprising eight contiguous amino acids of SEQ ID NO:132;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:131.

Preferably, such polynucleotide comprises the nucleotide sequence of SEQ ID NO:131 from nucleotide 914 to nucleotide 2353; the nucleotide sequence of SEQ ID NO:131 from nucleotide 1793 to nucleotide 2353; the nucleotide sequence of SEQ ID NO:131 from nucleotide 1037 to nucleotide 1260; the nucleotide sequence of the full-length protein coding sequence of clone CT748_{—}2 deposited with the ATCC under accession number 98271; or the nucleotide sequence of a mature protein coding sequence of clone CT748_{—}2 deposited with the ATCC under accession number 98271. In other preferred embodiments, the polynucleotide encodes the full-length or a mature protein encoded by the cDNA insert of clone CT748_{—}2-deposited with the ATCC under accession number 98271. In yet other preferred embodiments, the present invention provides a polynucleotide encoding a protein comprising the amino add sequence of SEQ ID NO:132 from amino acid 22 to amino acid 116. In further preferred embodiments, the present invention provides a polynucleotide encoding a protein comprising a fragment of the amino acid sequence of SEQ ID NO:132 having biological activity, the fragment preferably comprising eight (more preferably twenty, most preferably thirty) contiguous amino adds of SEQ ID NO:132, or a polynucleotide encoding a protein comprising a fragment of the amino acid sequence of SEQ ID NO:132 having biological activity, the fragment comprising the amino add sequence from amino add 234 to amino acid 243 of SEQ ID NO:132

Other embodiments provide the gene corresponding to the cDNA sequence of SEQ ID NO:131.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:131, but excluding the poly(A) tail at the 3′ end of SEQ ID NO:131; and
      (ab) the nucleotide sequence of the cDNA insert of clone CT748_{—}2 deposited with the ATCC under accession number 98271;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:131, but excluding the poly(A) tail at the 3′ end of SEQ ID NO:131; and
      (bb) the nucleotide sequence of the cDNA insert of clone CI748_{—}2 deposited with the ATCC under accession number 98271;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:131, and extending contiguously from a nucleotide sequence corresponding to the 5′ end of SEQ ID NO:131 to a nucleotide sequence corresponding to the 3′ end of SEQ ID NO:131, but excluding the poly(A) tail at the 3′ end of SEQ ID NO:131. Also preferably the polynucleotide isolated according to the above process comprises a nucleotide sequence corresponding to the cDNA sequence of SEQ ID NO:131 from nucleotide 914 to nucleotide 2353, and extending contiguously from a nucleotide sequence corresponding the 5′ end of said sequence of SEQ ID NO:131 from nucleotide 914 to nucleotide 2353, to a nucleotide sequence corresponding to the 3′ end of said sequence of SEQ ID NO:131 from nucleotide 914 to nucleotide 2353. Also preferably the polynucleotide isolated according to the above process comprises a nucleotide sequence corresponding to the cDNA sequence of SEQ ID NO:131 from nucleotide 1793 to nucleotide 2353, and extending contiguously from a nucleotide sequence corresponding to the 5′ end of said sequence of SEQ ID NO:131 from nucleotide 1793 to nucleotide 2353, to a nucleotide sequence corresponding to the 3′ end of said sequence of SEQ ID NO:131 from nucleotide 1793 to nucleotide 2353. Also preferably the polynucleotide isolated according to the above process comprises a nucleotide sequence corresponding to the cDNA sequence of SEQ ID NO:131 from nucleotide 1037 to nucleotide 1260, and extending contiguously from a nucleotide sequence corresponding to the 5′ end of said sequence of SEQ ID NO:131 from nucleotide 1037 to nucleotide 1260, to a nucleotide sequence corresponding to the 3′ end of said sequence of SEQ ID NO:131 from nucleotide 1037 to nucleotide 1260.

In other embodiments, the present invention provides a composition comprising a protein, wherein said protein comprises an amino acid sequence selected from the group consisting of:
(a) the amino add sequence of SEQ ID NO:132;
(b) the amino add sequence of SEQ ID NO:132 from amino acid 22 to amino acid 116;
(c) a fragment of the amino add sequence of SEQ ID NO:132, the fragment comprising eight contiguous amino adds of SEQ ID NO:132; and
(d) the amino acid sequence encoded by the cDNA insert of clone CT748_{—}2 deposited with the ATCC under accession number 98271;
   the protein being substantially free from other mammalian proteins. Preferably such protein comprises the amino acid sequence of SEQ ID NO:132 or the amino acid sequence of SEQ ID NO:132 from amino add 22 to amino acid 116. In further preferred embodiments, the present invention provides a protein comprising a fragment of the amino add sequence of SEQ ID NO:132 having biological activity, the fragment preferably comprising eight (more preferably twenty, most preferably thirty) contiguous amino acids of SEQ ID NO:132, or a protein comprising a fragment of the o add sequence of SEQ ID NO:132 having biological activity, the fragment comprising the amino acid sequence from amino acid 234 to amino acid 243 of SEQ ID NO:132

In one embodiment, the present invention provides a composition comprising an isolated polynucleotide selected from the group consisting of:
(a) a polynucleotide comprising the nucleotide sequence of SEQ ID NO:133;
(b) a polynucleotide comprising the nucleotide sequence of SEQ ID NO:133 from nucleotide 22 to nucleotide 462;
(c) a polynucleotide comprising the nucleotide sequence of the full-length protein coding sequence of clone AJ1_{—}1 deposited with the ATCC under accession number 98278;
(d) a polynucleotide encoding the full-length protein encoded by the cDNA insert of clone AJ1_{—}1 deposited with the ATCC under accession number 98278;
(e) a polynucleotide comprising the nucleotide sequence of a mature protein coding sequence of clone AJ1_{—}1 deposited with the ATCC under accession number 98278;
(f) a polynucleotide encoding a mature protein encoded by the cDNA insert of clone AJ1_{—}1 deposited with the ATCC under accession number 98278;
(g) a polynucleotide encoding a protein comprising the amino acid sequence of SEQ ID NO:134;
(h) a polynucleotide encoding a protein comprising a fragment of the amino acid sequence of SEQ ID NO:134 having biological activity, the fragment comprising eight contiguous amino acids of SEQ ID NO:134;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:133.

Preferably, such polynucleotide comprises the nucleotide sequence of SEQ ID NO:133 from nucleotide 22 to nucleotide 462; the nucleotide sequence of the full-length protein coding sequence of cone AJ1_{—}1 deposited with e ATCC under accession number 98278; or the nucleotide sequence of a mature protein coding sequence of clone AJ1_{—}1 deposited with the ATCC under accession number 98278. In other preferred embodiments, the polynucleotide encodes the full-length or a mature protein encoded by the cDNA insert of clone AJ1_{—}1 deposited with the ATCC under accession number 98278. In yet other preferred embodiments, the present invention provides a polynucleotide encoding a protein comprising the amino acid sequence of SEQ ID NO:134 from amino acid 52 to amino add 147. In further preferred embodiments, the present invention provides a polynucleotide encoding a protein comprising a fragment of the amino acid sequence of SEQ ID NO:134 having biological activity, the fragment preferably comprising eight (more preferably twenty, most preferably thirty) contiguous amino adds of SEQ ID NO:134, or a polynucleotide encoding a protein comprising a fragment of the amino add sequence of SEQ ID NO:134 having biological activity, the fragment comprising the amino acid sequence from amino acid 68 to amino acid 77 of SEQ ID NO:134.

Other embodiments provide the gene corresponding to the cDNA sequence of SEQ ID NO:133 and SEQ ID NO:135.

Further embodiments of the invention provide isolated polynucleotides produced according to a process selected from the group consisting of.
(a) a process comprising the steps of:
   (i) preparing one or more polynucleotide probes that hybridize in 6×SSC at 65 degrees C to a nucleotide sequence selected from the group consisting of:
      (aa) SEQ ID NO 133;
      (ab) SEQ ID NO:135, but excluding the poly(A) tail at the 3′ end of SEQ ID NO:135; and
      (ac) the nucleotide sequence of the cDNA insert of clone AJ1_{—}1 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:133;
      (bb) SEQ ID NO:135, but excluding the poly(A) tail at he 3′ end of SEQ ID NO:135; and
      (bc) the nucleotide sequence of the cDNA insert of clone AJ1_{—}1 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:133 and SEQ ID NO:135, and extending contiguously from a nucleotide sequence corresponding to the 5′ end of SEQ ID NO:133 to a nucleotide sequence corresponding to the 3′ end of SEQ ID NO:135, but excluding the poly(A) tail at the 3′ end of SEQ ID NO:135. Also preferably the polynucleotide isolated according to the above process comprises a nucleotide sequence corresponding to the cDNA sequence of SEQ ID NO:133, and extending contiguously from a nucleotide sequence corresponding to the 5′ end of SEQ ID NO:133 to a nucleotide sequence corresponding to the 3′ end of SEQ ID NO:133. Also preferably the polynucleotide isolated according to the above process comprises a nucleotide sequence corresponding to the cDNA sequence of SEQ ID NO:133 from nucleotide 22 to nucleotide 462, and extending contiguously from a nucleotide sequence corresponding to the 5′ end of said sequence of SEQ ID NO:133 from nucleotide 22 to nucleotide 462, to a nucleotide sequence corresponding to the 3′ end of said sequence of SEQ ID NO:133 from nucleotide 22 to nucleotide 462.

In other embodiments, the present invention provides a composition comprising a protein, wherein said protein comprises an amino add sequence selected from the group consisting of:
(a) the amino acid sequence of SEQ ID NO:134;
(b) the amino acid sequence of SEQ ID NO:134 from amino acid 52 to amino acid 147;
(c) a fragment of the amino acid sequence of SEQ ID NO:134, the fragment comprising eight contiguous amino acids of SEQ ID NO:134; and
(d) the amino acid sequence encoded by the cDNA insert of clone AJ1_{—}1 deposited with the ATCC under accession number 98278;
   the protein being substantially free from other mammalian proteins. Preferably such protein comprises the amino acid sequence of SEQ ID NO:134 or the amino acid sequence of SEQ ID NO:134 from amino acid 52 to amino acid 147. In further preferred embodiments, the present invention provides a protein comprising a fragment of the amino acid sequence of SEQ ID NO:134 having biological activity, the fragment preferably comprising eight (more preferably twenty, most preferably thirty) contiguous amino adds of SEQ ID NO:134, or a protein comprising a fragment of the amino acid sequence of SEQ ID NO:134 having biological activity, the fragment comprising the amino acid sequence from amino acid 68 to amino acid 77 of SEQ ID NO:134.

In one embodiment, the present invention provides a composition comprising an isolated polynucleotide selected from the group consisting of:
(a) a polynucleotide comprising the nucleotide sequence of SEQ ID NO:136;
(b) a polynucleotide comprising the nucleotide sequence of SEQ ID NO:136 from nucleotide 7 to nucleotide 1647;
(c) a polynucleotide comprising the nucleotide sequence of SEQ ID NO:136 from nucleotide 1 to nucleotide 305;
(d) a polynucleotide comprising the nucleotide sequence of the fill-length protein coding sequence of clone AQ73_{—}3 deposited with the ATCC under accession number 98278;
(e) a polynucleotide encoding the full-length protein encoded by the cDNA insert of clone AQ73_{—}3 deposited with the ATCC under accession number 98278;
(f) a polynucleotide comprising the nucleotide sequence of a mature protein coding sequence of clone AQ73_{—}3 deposited with the ATCC under accession number 98278;
(g) a polynucleotide encoding a mature protein encoded by the cDNA insert of clone AQ73_{—}3 deposited with the ATCC under accession number 98278;
(h) a polynucleotide encoding a protein comprising the amino acid sequence of SEQ ID NO:137;
(i) a polynucleotide encoding a protein comprising a fragment of the amino acid sequence of SEQ ID NO:137 having biological activity, the fragment comprising eight contiguous amino acids of SEQ ID NO:137;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:136.

Preferably, such polynucleotide comprises the nucleotide sequence of SEQ ID NO:136 from nucleotide 7 to nucleotide 1647; the nucleotide sequence of SEQ ID NO:136 from nucleotide 1 to nucleotide 305; the nucleotide sequence of the full-length protein coding sequence of clone AQ73_{—}3 deposited with the ATCC under accession number 98278; or the nucleotide sequence of a mature protein coding sequence of clone AQ73_{—}3 deposited with the ATCC under accession number 98278. In other preferred embodiments, the polynucleotide encodes the full-length or a mature protein encoded by the cDNA insert of clone AQ73_{—}3 deposited with the ATCC under accession number 98278. In yet other preferred embodiments, the present invention provides a polynucleotide encoding a protein comprising the amino acid sequence of SEQ ID NO:137 from amino acid 1 to amino acid 68. In further preferred embodiments, the present invention provides a polynucleotide encoding a protein comprising a fragment of the amino acid sequence of SEQ ID NO:137 having biological activity, the fragment preferably comprising eight (more preferably twenty, most preferably thirty) contiguous amino acids of SEQ ID NO:137, or a polynucleotide encoding a protein comprising a fragment of the amino acid sequence of SEQ ID NO:137 having biological activity, the fragment comprising the amino acid sequence from amino acid 268 to amino acid 277 of SEQ ID NO:137.

Other embodiments provide the gene corresponding to the cDNA sequence of SEQ ID NO:136.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:136, but excluding the poly(A) tail at the 3′ end of SEQ ID NO:136; and
      (ab) the nucleotide sequence of the cDNA insert of clone AQ73_{—}3 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:136, but excluding the poly(A) tail at the 3′ end of SEQ ID NO:136; and
      (bb) the nucleotide sequence of the cDNA insert of clone AQ73_{—}3 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:136, and extending contiguously from a nucleotide sequence corresponding to the 5′ end of SEQ ID NO:136 to a nucleotide sequence corresponding to the 3′ end of SEQ ID NO:136, but excluding the poly(A) tail at the 3′ end of SEQ ID NO:136. Also preferably the polynucleotide isolated according to the above process comprises a nucleotide sequence corresponding to the cDNA sequence of SEQ ID NO:136 from nucleotide 7 to nucleotide 1647, and extending contiguously from a nucleotide sequence corresponding to the 5′ end of said sequence of SEQ ID NO:136 from nucleotide 7 to nucleotide 1647, to a nucleotide sequence corresponding to the 3′ end of said sequence of SEQ ID NO:136 from nucleotide 7 to nucleotide 1647. Also preferably the polynucleotide isolated according to the above process comprises a nucleotide sequence corresponding to the cDNA sequence of SEQ ID NO:136 from nucleotide 1 to nucleotide 305, and extending contiguously from a nucleotide sequence corresponding to the 5′ end of said sequence of SEQ ID NO:136 from nucleotide 1 to nucleotide 305, to a nucleotide sequence corresponding to the 3′ end of said sequence of SEQ ID NO:136 from nucleotide 1 to nucleotide 305.

In other embodiments, the present invention provides a composition comprising a protein, wherein said protein comprises an amino acid sequence selected from the group consisting of:
(a) the amino acid sequence of SEQ ID NO:137;
(b) the amino acid sequence of SEQ ID NO:137 from amino acid 1 to amino acid 68;
(c) a fragment of the amino acid sequence of SEQ ID NO:137, the fragment comprising eight contiguous amino acids of SEQ ID NO:137; and
(d) the amino acid sequence encoded by the cDNA insert of clone AQ73_{—}3 deposited with the ATCC under accession number 98278;
   the protein being substantially free from other mammalian proteins. Preferably such protein comprises the amino acid sequence of SEQ ID NO:137 or the amino acid sequence of SEQ ID NO:137 from amino add 1 to amino acid 68. In further preferred embodiments, the present invention provides a protein comprising a fragment of the amino acid sequence of SEQ ID NO:137 having biological activity, the fragment preferably comprising eight (more preferably twenty, most preferably thirty) contiguous amino acids of SEQ ID NO:137, or a protein comprising a fragment of the amino acid sequence of SEQ ID NO:137 having biological activity, the fragment comprising the amino acid sequence from amino acid 268 to amino acid 277 of SEQ ID NO:137.

In one embodiment, the present invention provides a composition comprising an isolated polynucleotide selected from the group consisting of:
(a) a polynucleotide comprising the nucleotide sequence of SEQ ID NO:138;
(b) a polynucleotide comprising the nucleotide sequence of SEQ ID NO:138 from nucleotide 62 to nucleotide 757;
(c) a polynucleotide comprising the nucleotide sequence of SEQ ID NO:138 from nucleotide 357 to nucleotide 703;
(d) a polynucleotide comprising the nucleotide sequence of the full-length protein coding sequence of clone BG142_{—}1 deposited with the ATCC under accession number 98278;
(e) a polynucleotide encoding the full-length protein encoded by the cDNA insert of clone BG142_{—}1 deposited with the ATCC under accession number 98278;
(f) a polynucleotide comprising the nucleotide sequence of a mature protein coding sequence of clone BG142_{—}1 deposited with the ATCC under accession number 98278;
(g) a polynucleotide encoding a mature protein encoded by the cDNA insert of clone BG142_{—}1 deposited with the ATCC under accession number 98278;
(h) a polynucleotide encoding a protein comprising the amino acid sequence of SEQ ID NO:139;
(i) a polynucleotide encoding a protein comprising a fragment of the amino acid sequence of SEQ ID NO:139 having biological activity, the fragment comprising eight contiguous amino acids of SEQ ID NO:139;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:138.

Preferably, such polynucleotide comprises the nucleotide sequence of SEQ ID NO:138 from nucleotide 62 to nucleotide 757; the nucleotide sequence of SEQ ID NO:138 from nucleotide 357 to nucleotide 703; the nucleotide sequence of the full-length protein coding sequence of clone BG142_{—}1 deposited with the ATCC under accession number 98278; or the nucleotide sequence of a mature protein coding sequence of clone BG142_{—}1 deposited with the ATCC under accession number 98278. In other preferred embodiments, the polynucleotide encodes the full-length or a mature protein encoded by the cDNA insert of clone BG142_{—}1 deposited with the ATCC under accession number 98278. In yet other preferred embodiments, the present invention provides a polynucleotide encoding a protein comprising the amino acid sequence of SEQ ID NO:139 from amino acid 184 to amino acid 214. In further preferred embodiments, the present invention provides a polynucleotide encoding a protein comprising a fragment of the amino acid sequence of SEQ ID NO:139 having biological activity, the fragment preferably comprising eight (more preferably twenty, most preferably thirty) contiguous amino acids of SEQ ID NO:139, or a polynucleotide encoding a protein comprising a fragment of the amino acid sequence of SEQ ID NO:139 having biological activity, the fragment comprising the amino add sequence from amino acid 111 to amino add 120 of SEQ ID NO:139.

Other embodiments provide the gene corresponding to the cDNA sequence of SEQ ID NO:138.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:138, but excluding the poly(A) tail at the 3′ end of SEQ ID NO:138; and
      (ab) the nucleotide sequence of the cDNA insert of clone BG142_{—}1 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:138, but excluding the poly(A) tail at the 3′ end of SEQ ID NO:138; and
      (bb) the nucleotide sequence of the cDNA insert of clone BG14211 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:138, and extending contiguously from a nucleotide sequence corresponding to the 5′ end of SEQ ID NO:138 to a nucleotide sequence corresponding to the 3′ end of SEQ ID NO:138, but excluding the poly(A) tail at the 3′ end of SEQ ID NO:138. Also preferably the polynucleotide isolated according to the above process comprises a nucleotide sequence corresponding to the cDNA sequence of SEQ ID NO:138 from nucleotide 62 to nucleotide 757, and extending contiguously from a nucleotide sequence corresponding to the 5′ end of said sequence of SEQ ID NO:138 from nucleotide 62 to nucleotide 757, to a nucleotide sequence corresponding to the 3′ end of said sequence of SEQ ID NO:138 from nucleotide 62 to nucleotide 757. Also preferably the polynucleotide isolated according to the above process comprises a nucleotide sequence corresponding to the cDNA sequence of SEQ ID NO:138 from nucleotide 357 to nucleotide 703, and extending contiguously from a nucleotide sequence corresponding to the 5′ end of said sequence of SEQ ID NO:138 from nucleotide 357 to nucleotide 703, to a nucleotide sequence corresponding to the 3′ end of said sequence of SEQ ID NO:138 from nucleotide 357 to nucleotide 703. In other embodiments, the present invention provides a composition comprising a protein, wherein said protein comprises an amino acid sequence selected from the group consisting of:
(a) the amino acid sequence of SEQ ID NO:139;
(b) the amino acid sequence of SEQ ID NO:139 from amino acid 184 to amino acid 214;
(c) a fragment of the amino acid sequence of SEQ ID NO:139, the fragment comprising eight contiguous amino acids of SEQ ID NO:139; and
(d) the amino acid sequence encoded by the cDNA insert of clone BG142_{—}1 deposited with the ATCC under accession number 98278;
   the protein being substantially free from other mammalian proteins. Preferably such protein comprises the amino acid sequence of SEQ ID NO:139 or the amino acid sequence of SEQ ID NO:139 from amino acid 184 to amino acid 214. In further preferred embodiments, the present invention provides a protein comprising a fragment of the amino acid sequence of SEQ ID NO:139 having biological activity, the fragment preferably comprising eight (more preferably twenty, most preferably thirty) contiguous amino acids of SEQ ID NO:139, or a protein comprising a fragment of the amino acid sequence of SEQ ID NO:139 having biological activity, the fragment comprising the amino add sequence from amino acid 111 to amino acid 120 of SEQ ID NO:139.

In one embodiment, the present invention provides a composition comprising an isolated polynucleotide selected from the group consisting of:
(a) a polynucleotide comprising the nucleotide sequence of SEQ ID NO:140;
(b) a polynucleotide comprising the nucleotide sequence of SEQ ID NO:140 from nucleotide 404 to nucleotide 535;
(c) a polynucleotide comprising the nucleotide sequence of SEQ ID NO:140 from nucleotide 1 to nucleotide 666;
(d) a polynucleotide comprising the nucleotide sequence of the full-length protein coding sequence of clone BV66_{—}1 deposited with the ATCC under accession number 98278;
(e) a polynucleotide encoding the full-length protein encoded by the cDNA insert of clone BV66_{—}1 deposited with the ATCC under accession number 98278;
(f) a polynucleotide comprising the nucleotide sequence of a mature protein coding sequence of clone BV66_{—}1 deposited with the ATCC under accession number 98278;
(g) a polynucleotide encoding a mature protein encoded by the cDNA insert of clone BV66_{—}1 deposited with the ATCC under accession number 98278;
(h) a polynucleotide encoding a protein comprising the amino acid sequence of SEQ ID NO:141;
(i) a polynucleotide encoding a protein comprising a fragment of the amino acid sequence of SEQ ID NO:141 having biological activity, the fragment comprising eight contiguous amino acids of SEQ ID NO:141;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:140.

Preferably, such polynucleotide comprises the nucleotide sequence of SEQ ID NO:140 from nucleotide 404 to nucleotide 535; the nucleotide sequence of SEQ ID NO:140 from nucleotide 1 to nucleotide 666; the nucleotide sequence of the full-length protein coding sequence of clone BV66_{—}1 deposited with the ATCC under accession number 98278; or the nucleotide sequence of a mature protein coding sequence of clone BV66_{—}1 deposited with the ATCC under accession number 98278. In other preferred embodiments, the polynucleotide encodes the full-length or a mature protein encoded by the cDNA insert of clone BV66_{—}1 deposited with the ATCC under accession number 98278. In yet other preferred embodiments, the present invention provides a polynucleotide encoding a protein comprising the amino acid sequence of SEQ ID NO:141 from amino acid 1 to amino acid 38. In further preferred embodiments, the present invention provides a polynucleotide encoding a protein comprising a fragment of the amino acid sequence of SEQ ID NO:141 having biological activity, the fragment preferably comprising eight (more preferably twenty, most preferably thirty) contiguous amino acids of SEQ ID NO:141, or a polynucleotide encoding a protein comprising a fragment of the amino acid sequence of SEQ ID NO:141 having biological activity, the fragment comprising the amino acid sequence from amino acid 17 to amino acid 26 of SEQ ID NO:141.

Other embodiments provide the gene corresponding to the cDNA sequence of SEQ ID NO:140.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:140, but excluding the poly(A) tail at the 3′ end of SEQ ID NO:140; and
      (ab) the nucleotide sequence of the cDNA insert of clone BV66_{—}1 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:140, but excluding the poly(A) tail at the 3′ end of SEQ ID NO:140; and
      (bb) the nucleotide sequence of the cDNA insert of clone BV66_{—}1 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:140, and extending contiguously from a nucleotide sequence corresponding to the 5′ end of SEQ ID NO:140 to a nucleotide sequence corresponding to the 3′ end of SEQ ID NO:140, but excluding the poly(A) tail at the 3′ end of SEQ ID NO:140. Also preferably the polynucleotide isolated according to the above process comprises a nucleotide sequence corresponding to the cDNA sequence of SEQ ID NO:140 from nucleotide 404 to nucleotide 535, and extending contiguously from a nucleotide sequence corresponding to the 5′ end of said sequence of SEQ ID NO:140 from nucleotide 404 to nucleotide 535, to a nucleotide sequence corresponding to the 3′ end of said sequence of SEQ ID NO:140 from nucleotide 404 to nucleotide 535. Also preferably the polynucleotide isolated according to the above process comprises a nucleotide sequence corresponding to the cDNA sequence of SEQ ID NO:140 from nucleotide 1 to nucleotide 666, and extending contiguously from a nucleotide sequence corresponding to the 5′ end of said sequence of SEQ ID NO:140 from nucleotide 1 to nucleotide 666, to a nucleotide sequence corresponding to the 3′ end of said sequence of SEQ ID NO:140 from nucleotide 1 to nucleotide 666.

In other embodiments, the present invention provides a composition comprising a protein, wherein said protein comprises an amino acid sequence selected from the group consisting of:
(a) the amino acid sequence of SEQ ID NO:141;
(b) the amino acid sequence of SEQ ID NO:141 from amino acid 1 to amino acid 38;
(c) a fragment of the amino acid sequence of SEQ ID NO:141, the fragment comprising eight contiguous amino acids of SEQ ID NO:141; and
(d) the amino acid sequence encoded by the cDNA insert of clone BV66_{—}1 deposited with the ATCC under accession number 98278;
   the protein being substantially free from other mammalian proteins. Preferably such protein comprises the amino acid sequence of SEQ ID NO:141 or the amino acid sequence of SEQ ID NO:141 from amino acid 1 to amino add 38. In further preferred embodiments, the present invention provides a protein comprising a fragment of the amino acid sequence of SEQ ID NO:141 having biological activity, the fragment preferably comprising eight (more preferably twenty, most preferably thirty) contiguous amino acids of SEQ ID NO:141, or a protein comprising a fragment of the amino add sequence of SEQ ID NO:141 having biological activity, the fragment comprising the amino acid sequence from amino acid 17 to amino acid 26 of SEQ ID NO:141.

In one embodiment, the present invention provides a composition comprising an isolated polynucleotide selected from the group consisting of:
(a) a polynucleotide comprising the nucleotide sequence of SEQ ID NO:142;
(b) a polynucleotide comprising the nucleotide sequence of SEQ ID NO:142 from nucleotide 1204 to nucleotide 1389;
(c) a polynucleotide comprising the nucleotide sequence of SEQ ID NO:142 from nucleotide 881 to nucleotide 1380;
(d) a polynucleotide comprising the nucleotide sequence of the full-length protein coding sequence of clone BV291_{—}3 deposited with the ATCC under accession number 98278;
(e) a polynucleotide encoding the full-length protein encoded by the cDNA insert of clone BV291_{—}3 deposited with the ATCC under accession number 98278;
(f) a polynucleotide comprising the nucleotide sequence of a mature protein coding sequence of clone BV2913 deposited with the ATCC under accession number 98278;
(g) a polynucleotide encoding a mature protein encoded by the cDNA insert of clone BV2913 deposited with the ATCC under accession number 98278;
(h) a polynucleotide encoding a protein comprising the amino add sequence of SEQ ID NO:143;
(i) a polynucleotide encoding a protein comprising a fragment of the amino acid sequence of SEQ ID NO:143 having biological activity, the fragment comprising eight contiguous amino acids of SEQ ID NO:143;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:142.

Preferably, such polynucleotide comprises the nucleotide sequence of SEQ ID NO:142 from nucleotide 1204 to nucleotide 1389; the nucleotide sequence of SEQ ID NO:142 from nucleotide 881 to nucleotide 1380; the nucleotide sequence of the full-length protein coding sequence of clone BV291_{—}3 deposited with the ATCC under accession number 98278; or the nucleotide sequence of a mature protein coding sequence of clone BV291_{—}3 deposited with the ATCC under accession number 98278. In other preferred embodiments, the polynucleotide encodes the full-length or a mature protein encoded by the cDNA insert of clone BV291_{—}3 deposited with the ATCC under accession number 98278. In yet other preferred embodiments, the present invention provides a polynucleotide encoding a protein comprising the amino add sequence of SEQ ID NO:143 from amino acid 1 to amino acid 59. In further preferred embodiments, the present invention provides a polynucleotide encoding a protein comprising a fragment of the amino acid sequence of SEQ ID NO:143 having biological activity, the fragment preferably comprising eight (more preferably twenty, most preferably thirty) contiguous amino acids of SEQ ID NO:143, or a polynucleotide encoding a protein comprising a fragment of the amino add sequence of SEQ ID NO:143 having biological activity, the fragment comprising the amino acid sequence from amino acid 26 to amino add 35 of SEQ ID NO:143.

Other embodiments provide the gene corresponding to the cDNA sequence of SEQ ID NO:142.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:142, but excluding the poly(A) tail at the 3′ end of SEQ ID NO:142; and
      (ab) the nucleotide sequence of the cDNA insert of clone BV291_{—}3 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:142, but excluding the poly(A) tail at the 3′ end of SEQ ID NO:142; and
      (bb) the nucleotide sequence of the cDNA insert of clone BV291_{—}3 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:142, and extending contiguously from a nucleotide sequence corresponding to the 5′ end of SEQ ID NO:142 to a nucleotide sequence corresponding to the 3′ end of SEQ ID NO:142, but excluding the poly(A) tail at the 3′ end of SEQ ID NO:142. Also preferably the polynucleotide isolated according to the above process comprises a nucleotide sequence corresponding to the cDNA sequence of SEQ ID NO:142 from nucleotide 1204 to nucleotide 1389, and extending contiguously from a nucleotide sequence corresponding to the 5′ end of said sequence of SEQ ID NO:142 from nucleotide 1204 to nucleotide 1389, to a nucleotide sequence corresponding to the 3′ end of said sequence of SEQ ID NO:142 from nucleotide 1204 to nucleotide 1389. Also preferably the polynucleotide isolated according to the above process comprises a nucleotide sequence corresponding to the cDNA sequence of SEQ ID NO:142 from nucleotide 881 to nucleotide 1380, and extending contiguously from a nucleotide sequence corresponding to the 5′ end of said sequence of SEQ ID NO:142 from nucleotide 881 to nucleotide 1380, to a nucleotide sequence corresponding to the 3′ end of said sequence of SEQ ID NO:142 from nucleotide 881 to nucleotide 1380.

In other embodiments, the present invention provides a composition comprising a protein, wherein said protein comprises an amino acid sequence selected from the group consisting of:
(a) the amino acid sequence of SEQ ID NO:143;
(b) the amino add sequence of SEQ ID NO:143 from amino acid 1 to amino acid 59;
(c) a fragment of the amino acid sequence of SEQ ID NO:143, the fragment comprising eight contiguous amino acids of SEQ ID NO:143; and
(d) the amino acid sequence encoded by the cDNA insert of clone BV291_{—}3 deposited with the ATCC under accession number 98278;
   the protein being substantially free from other mammalian proteins. Preferably such protein comprises the amino acid sequence of SEQ ID NO:143 or the amino acid sequence of SEQ ID NO:143 from amino acid 1 to amino acid 59. In further preferred embodiments, the present invention provides a protein comprising a fragment of the amino acid sequence of SEQ ID NO:143 having biological activity, the fragment preferably comprising eight (more preferably twenty, most preferably thirty) contiguous amino acids of SEQ ID NO:143, or a protein comprising a fragment of the amino acid sequence of SEQ ID NO:143 having biological activity, the fragment comprising the amino acid sequence from amino acid 26 to amino acid 35 of SEQ ID NO:143.

In one embodiment, the present invention provides a composition comprising an isolated polynucleotide selected from the group consisting of:
(a) a polynucleotide comprising the nucleotide sequence of SEQ ID NO:144;
(b) a polynucleotide comprising the nucleotide sequence of SEQ ID NO:144 from nucleotide 189 to nucleotide 1115;
(c) a polynucleotide comprising the nucleotide sequence of SEQ ID NO:144 from nucleotide 1 to nucleotide 451;
(d) a polynucleotide comprising the nucleotide sequence of the full-length protein coding sequence of clone CK201_{—}1 deposited with the ATCC under accession number 98278;
(e) a polynucleotide encoding the full-length protein encoded by the cDNA insert of clone CK201_{—}1 deposited with the ATCC under accession number 98278;
(f) a polynucleotide comprising the nucleotide sequence of a mature protein coding sequence of clone CK201_{—}1 deposited with the ATCC under accession number 98278;
(g) a polynucleotide encoding a mature protein encoded by the cDNA insert of clone CK201_{—}1 deposited with the ATCC under accession number 98278;
(h) a polynucleotide encoding a protein comprising the amino acid sequence of SEQ ID NO:145;
(i) a polynucleotide encoding a protein comprising a fragment of the amino acid sequence of SEQ ID NO:145 having biological activity, the fragment comprising eight contiguous amino acids of SEQ ID NO:145;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:144.

Preferably, such polynucleotide comprises the nucleotide sequence of SEQ ID NO:144 from nucleotide 189 to nucleotide 1115; the nucleotide sequence of SEQ ID 25. NO:144 from nucleotide 1 to nucleotide 451; the nucleotide sequence of the full-length protein coding sequence of clone CK201_{—}1 deposited with the ATCC under accession number 98278; or the nucleotide sequence of a mature protein coding sequence of clone CK201_{—}1 deposited with the ATCC under accession number 98278. In other preferred embodiments, the polynucleotide encodes the full-length or a mature protein encoded by the cDNA insert of clone CK201_{—}1 deposited with the ATCC under accession number 98278. In yet other preferred embodiments, the present invention provides a polynucleotide encoding a protein comprising the amino add sequence of SEQ ID NO:145 from amino acid 1 to amino acid 88. In further preferred embodiments, the present invention provides a polynucleotide encoding a protein comprising a fragment of the amino add sequence of SEQ ID NO:145 having biological activity, the fragment preferably comprising eight (more preferably twenty, most preferably thirty) contiguous amino acids of SEQ ID NO:145, or a polynucleotide encoding a protein comprising a fragment of the amino acid sequence of SEQ ID NO:145 having biological activity, the fragment comprising the amino acid sequence from amino add 149 to amino acid 158 of SEQ ID NO:145.

Other embodiments provide the gene corresponding to the cDNA sequence of SEQ ID NO:144.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:144, but excluding the poly(A) tail at the 3′ end of SEQ ID NO:144; and
      (ab) the nucleotide sequence of the cDNA insert of clone CK201_{—}1 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of.
      (ba) SEQ ID NO:144, but excluding the poly(A) tail at the 3′ end of SEQ ID NO:144; and
      (bb) the nucleotide sequence of the cDNA insert of clone CK201_{—}1 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:144, and extending contiguously from a nucleotide sequence corresponding to the 5′ end of SEQ ID NO:144 to a nucleotide sequence corresponding to the 3′ end of SEQ ID NO:144, but excluding the poly(A) tail at the 3′ end of SEQ ID NO:144. Also preferably the polynucleotide isolated according to the above process comprises a nucleotide sequence corresponding to the cDNA sequence of SEQ ID NO:144 from nucleotide 189 to nucleotide 1115, and extending contiguously from a nucleotide sequence corresponding to the 5′ end of said sequence of SEQ ID NO:144 from nucleotide 189 to nucleotide 1115, to a nucleotide sequence corresponding to the 3′ end of said sequence of SEQ ID NO:144 from nucleotide 189 to nucleotide 1115. Also preferably the polynucleotide isolated according to the above process comprises a nucleotide sequence corresponding to the cDNA sequence of SEQ ID NO:144 from nucleotide 1 to nucleotide 451, and extending contiguously from a nucleotide sequence corresponding to the 5′ end of said sequence of SEQ ID NO:144 from nucleotide 1 to nucleotide 451, to a nucleotide sequence corresponding to the 3′ end of said sequence of SEQ ID NO:144 from nucleotide 1 to nucleotide 451.

In other embodiments; the present invention provides a composition comprising a protein, wherein said protein comprises an amino acid sequence selected from the group consisting of:
(a) the amino acid sequence of SEQ ID NO:145;
(b) the amino acid sequence of SEQ ID NO:145 from amino acid 1 to amino acid 88;
(c) a fragment of the amino acid sequence of SEQ ID NO:145, the fragment comprising eight contiguous amino acids of SEQ ID NO:145; and
(d) the amino acid sequence encoded by the cDNA insert of clone CK201_{—}1 deposited with the ATCC under accession number 98278;
   the protein being substantially free from other mammalian proteins. Preferably such protein comprises the amino acid sequence of SEQ ID NO:145 or the amino acid sequence of SEQ ID NO:145 from amino acid 1 to amino acid 88. In further preferred embodiments, the present invention provides a protein comprising a fragment of the amino acid sequence of SEQ ID NO:145 having biological activity, the fragment preferably comprising eight (more preferably twenty, most preferably thirty) contiguous amino acids of SEQ ID NO:145, or a protein comprising a fragment of the amino acid sequence of SEQ ID NO:145 having biological activity, the fragment comprising the amino acid sequence from amino acid 149 to amino acid 158 of SEQ ID NO:145.

In one embodiment, the present invention provides a composition comprising an isolated polynucleotide selected from the group consisting of:
(a) a polynucleotide comprising the nucleotide sequence of SEQ ID NO:146;
(b) a polynucleotide comprising the nucleotide sequence of SEQ ID NO:146 from nucleotide 117 to nucleotide 923;
(c) a polynucleotide comprising the nucleotide sequence of SEQ ID NO:146 from nucleotide 174 to nucleotide 923;
(d) a polynucleotide comprising the nucleotide sequence of SEQ ID NO:146 from nucleotide 1 to nucleotide 316;
(e) a polynucleotide comprising the nucleotide sequence of the full-length protein coding sequence of clone CQ331_{—}2 deposited with the ATCC under accession number 98278;
(f) a polynucleotide encoding the full-length protein encoded by the cDNA insert of clone CQ331_{—}2 deposited with the ATCC under accession number 98278;
(g) a polynucleotide comprising the nucleotide sequence of a mature protein coding sequence of clone CQ331_{—}2 deposited with the ATCC under accession number 98278;
(h) a polynucleotide encoding a mature protein encoded by the cDNA insert of clone CQ331_{—}2 deposited with the ATCC under accession number 98278;
(i) a polynucleotide encoding a protein comprising the amino acid sequence of SEQ ID NO:147;
(j) a polynucleotide encoding a protein comprising a fragment of the amino acid sequence of SEQ ID NO:147 having biological activity, the fragment comprising eight contiguous amino acids of SEQ ID NO:147;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:146.

Preferably, such polynucleotide comprises the nucleotide sequence of SEQ ID NO:146 from nucleotide 117 to nucleotide 923; the nucleotide sequence of SEQ ID NO:146 from nucleotide 174 to nucleotide 923; the nucleotide sequence of SEQ ID NO:146 from nucleotide 1 to nucleotide 316; the nucleotide sequence of the full-length protein coding sequence of clone CQ331_{—}2 deposited with the ATCC under accession number 98278; or the nucleotide sequence of a mature protein coding sequence of clone CQ331_{—}2 deposited with the ATCC under accession number 98278. In other preferred embodiments, the polynucleotide encodes the full-length or a mature protein encoded by the cDNA insert of clone CQ331_{—}2 deposited with the ATCC under accession number 98278. In yet other preferred embodiments, the present invention provides a polynucleotide encoding a protein comprising the amino acid sequence of SEQ ID NO:147 from amino acid 1 to amino acid 57. In further preferred embodiments, the present invention provides a polynucleotide encoding a protein comprising a fragment of the amino acid sequence of SEQ ID NO:147 having biological activity, the fragment preferably comprising eight (more preferably twenty, most preferably thirty) contiguous amino acids of SEQ ID NO:147, or a polynucleotide encoding a protein comprising a fragment of the amino acid sequence of SEQ ID NO:147 having biological activity, the fragment comprising the amino acid sequence from amino acid 129 to amino acid 138 of SEQ ID NO:147.

Other embodiments provide the gene corresponding to the cDNA sequence of SEQ ID NO:146.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:146, but excluding the poly(A) tail at the 3′ end of SEQ ID NO:146; and
      (ab) the nucleotide sequence of the cDNA insert of clone CQ331_{—}2 deposited with the ATCC under accession number 98278;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:146, but excluding the poly(A) tail at the 3′ end of SEQ ID NO:146; and
      (bb) the nucleotide sequence of the cDNA insert of clone CQ331_{—}2 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:146, and extending contiguously from a nucleotide sequence corresponding to the 5′ end of SEQ ID NO:146 to a nucleotide sequence corresponding to the 3′ end of SEQ ID NO:146, but excluding the poly(A) tail at the 3′ end of SEQ ID NO:146. Also preferably the polynucleotide isolated according to the above process comprises a nucleotide sequence corresponding to the cDNA sequence of SEQ ID NO:146 from nucleotide 117 to nucleotide 923, and extending contiguously from a nucleotide sequence corresponding to the 5′ end of said sequence of SEQ ID NO:146 from nucleotide 117 to nucleotide 923, to a nucleotide sequence corresponding to the 3′ end of said sequence of SEQ ID NO:146 from nucleotide 117 to nucleotide 923. Also preferably the polynucleotide isolated according to the above process comprises a nucleotide sequence corresponding to the cDNA sequence of SEQ ID NO:146 from nucleotide 174 to nucleotide 923, and extending contiguously from a nucleotide sequence corresponding to the 5′ end of said sequence of SEQ ID NO:146 from nucleotide 174 to nucleotide 923, to a nucleotide sequence corresponding to the 3′ end of said sequence of SEQ ID NO:146 from nucleotide 174 to nucleotide 923. Also preferably the polynucleotide isolated according to the above process comprises a nucleotide sequence corresponding to the cDNA sequence of SEQ ID NO:146 from nucleotide 1 to nucleotide 316, and extending contiguously from a nucleotide sequence corresponding to the 5′ end of said sequence of SEQ ID NO:146 from nucleotide 1 to nucleotide 316, to a nucleotide sequence corresponding to the 3′ end of said sequence of SEQ ID NO:146 from nucleotide 1 to nucleotide 316.

In other embodiments, the present invention provides a composition comprising a protein, wherein said protein comprises an amino acid sequence selected from the group consisting of:
(a) the amino acid sequence of SEQ ID NO:147;
(b) the amino acid sequence of SEQ ID NO:147 from amino acid 1 to amino acid 57;
(c) a fragment of the amino acid sequence of SEQ ID NO:147, the fragment comprising eight contiguous amino acids of SEQ ID NO:147; and
(d) the amino acid sequence encoded by the cDNA insert of clone CQ331_{—}2 deposited with the ATCC under accession number 98278;
   the protein being substantially free from other mammalian proteins. Preferably such protein comprises the amino acid sequence of SEQ ID NO:147 or the amino acid sequence of SEQ ID NO:147 from amino acid 1 to amino acid 57. In further preferred embodiments, the present invention provides a protein comprising a fragment of the amino acid sequence of SEQ ID NO:147 having biological activity, the fragment preferably comprising eight (more preferably twenty, most preferably thirty) contiguous amino acids of SEQ ID NO:147, or a protein comprising a fragment of the amino acid sequence of SEQ ID NO:147 having biological activity, the fragment comprising the amino acid sequence from amino acid 129 to amino acid 138 of SEQ ID NO:147.

In one embodiment, the present invention provides a composition comprising an isolated polynucleotide selected from the group consisting of:
(a) a polynucleotide comprising the nucleotide sequence of SEQ ID NO:148;
(b) a polynucleotide comprising the nucleotide sequence of SEQ ID NO:148 from nucleotide 223 to nucleotide 483;
(c) a polynucleotide comprising the nucleotide sequence of SEQ ID NO:148 from nucleotide 22 to nucleotide 397;
(d) a polynucleotide comprising the nucleotide sequence of the full-length protein coding sequence of clone CT550_{—}1 deposited with the ATCC under accession number 98278;
(e) a polynucleotide encoding the full-length protein encoded by the cDNA insert of clone CT550_{—}1 deposited with the ATCC under accession number 98278;
(f) a polynucleotide comprising the nucleotide sequence of a mature protein coding sequence of clone CT550_{—}1 deposited with the ATCC under accession number 98278;
(g) a polynucleotide encoding a mature protein encoded by the cDNA insert of clone CT550_{—}1 deposited with the ATCC under accession number 98278;
(h) a polynucleotide encoding a protein comprising the amino acid sequence of SEQ ID NO:149;
(i) a polynucleotide encoding a protein comprising a fragment of the amino acid sequence of SEQ ID NO:149 having biological activity, the fragment comprising eight contiguous amino acids of SEQ ID NO:149;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:148.

Preferably, such polynucleotide comprises the nucleotide sequence of SEQ ID NO:148 from nucleotide 223 to nucleotide 483; the nucleotide sequence of SEQ ID NO:148 from nucleotide 22 to nucleotide 397; the nucleotide sequence of the full-length protein coding sequence of clone CT550_{—}1 deposited with the ATCC under accession number 98278; or the nucleotide sequence of a mature protein coding sequence of clone CT550_{—}1 deposited with the ATCC under accession number 98278. In other preferred embodiments, the polynucleotide encodes the full-length or a mature protein encoded by the cDNA insert of clone CT550_{—}1 deposited with the ATCC under accession number 98278. In yet other preferred embodiments, the present invention provides a polynucleotide encoding a protein comprising the amino acid sequence of SEQ ID NO:149 from amino acid 1 to amino acid 58. In further preferred embodiments, the present invention provides a polynucleotide encoding a protein comprising a fragment of the amino acid sequence of SEQ ID NO:149 having biological activity, the fragment preferably comprising eight (more preferably twenty, most preferably thirty) contiguous amino acids of SEQ ID NO:149, or a polynucleotide encoding a protein comprising a fragment of the amino acid sequence of SEQ ID NO:149 having biological activity, the fragment comprising the amino acid sequence from amino acid 38 to amino acid 47 of SEQ ID NO:149.

Other embodiments provide the gene corresponding to the cDNA sequence of SEQ ID NO:148.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:148, but excluding the poly(A) tail at the 3′ end of SEQ ID NO:148; and
      (ab) the nucleotide sequence of the cDNA insert of clone CT550_{—}1 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:148, but excluding the poly(A) tail at the 3′ end of SEQ ID NO:148; and
      (bb) the nucleotide sequence of the cDNA insert of clone CT550_{—}1 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(ii).
      Preferably the polynucleotide isolated according to the above process comprises a nucleotide sequence corresponding to the cDNA sequence of SEQ ID NO:148, and extending contiguously from a nucleotide sequence corresponding to the 5′ end of SEQ ID NO:148 to a nucleotide sequence corresponding to the 3′ end of SEQ ID NO:148, but excluding the poly(A) tail at the 3′ end of SEQ ID NO:148. Also preferably the polynucleotide isolated according to the above process comprises a nucleotide sequence corresponding to the cDNA sequence of SEQ ID NO:148 from nucleotide 223 to nucleotide 483, and extending contiguously from a nucleotide sequence corresponding to the 5′ end of said sequence of SEQ ID NO:148 from nucleotide 223 to nucleotide 483, to a nucleotide sequence corresponding to the 3′ end of said sequence of SEQ ID NO:148 from nucleotide 223 to nucleotide 483. Also preferably the polynucleotide isolated according to the above process comprises a nucleotide sequence corresponding to the cDNA sequence of SEQ ID NO:148 from nucleotide 22 to nucleotide 397, and extending contiguously from a nucleotide sequence corresponding to the 5′ end of said sequence of SEQ ID NO:148 from nucleotide 22 to nucleotide 397, to a nucleotide sequence corresponding to the 3′ end of said sequence of SEQ ID NO:148 from nucleotide 22 to nucleotide 397.

In other embodiments, the present invention provides a composition comprising a protein, wherein said protein comprises an amino acid sequence selected from the group consisting of:
(a) the amino acid sequence of SEQ ID NO:149;
(b) the amino acid sequence of SEQ ID NO:149 from amino acid 1 to amino acid 58;
(c) a fragment of the amino acid sequence of SEQ ID NO:149, the fragment comprising eight contiguous amino acids of SEQ ID NO:149; and
(d) the amino acid sequence encoded by the cDNA insert of clone CT550_{—}1 deposited with the ATCC under accession number 98278;
   the protein being substantially free from other mammalian proteins. Preferably such protein comprises the amino acid sequence of SEQ ID NO:149 or the amino acid sequence of SEQ ID NO:149 from amino acid 1 to amino acid 58. In further preferred embodiments, the present invention provides a protein comprising a fragment of the amino acid sequence of SEQ ID NO:149 having biological activity, the fragment preferably comprising eight (more preferably twenty, most preferably thirty) contiguous amino acids of SEQ ID NO:149, or a protein comprising a fragment of the amino acid sequence of SEQ ID NO:149 having biological activity, the fragment comprising the amino acid sequence from amino acid 38 to amino acid 47 of SEQ ID NO:149.

In one embodiment, the present invention provides a composition comprising an isolated polynucleotide selected from the group consisting of:
(a) a polynucleotide comprising the nucleotide sequence of SEQ ID NO:150;
(b) a polynucleotide comprising the nucleotide sequence of SEQ ID NO:150 from nucleotide 112 to nucleotide 969;
(c) a polynucleotide comprising the nucleotide sequence of SEQ ID NO:150 from nucleotide 154 to nucleotide 969;
(d) a polynucleotide comprising the nucleotide sequence of SEQ ID NO:150 from nucleotide 1 to nucleotide 423;
(e) a polynucleotide comprising the nucleotide sequence of the full-length protein coding sequence of clone CT585_{—}1 deposited with the ATCC under accession number 98278;
(f) a polynucleotide encoding the full-length protein encoded by the cDNA insert of clone CT585_{—}1 deposited with the ATCC under accession number 98278;
(g) a polynucleotide comprising the nucleotide sequence of a mature protein coding sequence of clone CT585_{—}1 deposited with the ATCC under accession number 98278;
(h) a polynucleotide encoding a mature protein encoded by the cDNA insert of clone CT585_{—}1 deposited with the ATCC under accession number 98278;
(i) a polynucleotide encoding a protein comprising the amino acid sequence of SEQ ID NO:151;
(j) a polynucleotide encoding a protein comprising a fragment of the amino acid sequence of SEQ ID NO:151 having biological activity, the fragment comprising eight contiguous amino acids of SEQ ID NO:151;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:150.

Preferably, such polynucleotide comprises the nucleotide sequence of SEQ ID NO:150 from nucleotide 112 to nucleotide 969; the nucleotide sequence of SEQ ID NO:150 from nucleotide 154 to nucleotide 969; the nucleotide sequence of SEQ ID NO:150 from nucleotide 1 to nucleotide 423; the nucleotide sequence of the full-length protein coding sequence of clone CT585_{—}1 deposited with the ATCC under accession number 98278; or the nucleotide sequence of a mature protein coding sequence of clone CT585_{—}1 deposited with the ATCC under accession number 98278. In other preferred embodiments, the polynucleotide encodes the full-length or a mature protein encoded by the cDNA insert of clone CT585_{—}1 deposited with the ATCC under accession number 98278. In yet other preferred embodiments, the present invention provides a polynucleotide encoding a protein comprising the amino acid sequence of SEQ ID NO:151 from amino acid 1 to amino acid 104. In further preferred embodiments, the present invention provides a polynucleotide encoding a protein comprising a fragment of the amino acid sequence of SEQ ID NO:151 having biological activity, the fragment preferably comprising eight (more preferably twenty, most preferably thirty) contiguous amino acids of SEQ ID NO:151, or a polynucleotide encoding a protein comprising a fragment of the amino acid sequence of SEQ ID NO:151 having biological activity, the fragment comprising the amino acid sequence from amino acid 138 to amino acid 147 of SEQ ID NO:151.

Other embodiments provide the gene corresponding to the cDNA sequence of SEQ ID NO:150.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:150, but excluding the poly(A) tail at the 3′ end of SEQ ID NO:150; and
      (ab) the nucleotide sequence of the cDNA insert of clone CT585_{—}1 deposited with the ATCC under accession number 98278;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:150, but excluding the poly(A) tail at the 3′ end of SEQ ID NO:150; and
      (bb) the nucleotide sequence of the cDNA insert of clone CT585_{—}1 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:150, and extending contiguously from a nucleotide sequence corresponding to the 5′ end of SEQ ID NO:150 to a nucleotide sequence corresponding to the 3′ end of SEQ ID NO:150, but excluding the poly(A) tail at the 3′ end of SEQ ID NO:150. Also preferably the polynucleotide isolated according to the above process comprises a nucleotide sequence corresponding to the cDNA sequence of SEQ ID NO:150 from nucleotide 112 to nucleotide 969, and extending contiguously from a nucleotide sequence corresponding to the 5′ end of said sequence of SEQ ID NO:150 from nucleotide 112 to nucleotide 969, to a nucleotide sequence corresponding to the 3′ end of said sequence of SEQ ID NO:150 from nucleotide 112 to nucleotide 969. Also preferably the polynucleotide isolated according to the above process comprises a nucleotide sequence corresponding to the cDNA sequence of SEQ ID NO:150 from nucleotide 154 to nucleotide 969, and extending contiguously from a nucleotide sequence corresponding to the 5′ end of said sequence of SEQ ID NO:150 from nucleotide 154 to nucleotide 969, to a nucleotide sequence corresponding to the 3′ end of said sequence of SEQ ID NO:150 from nucleotide 154 to nucleotide 969. Also preferably the polynucleotide isolated according to the above process comprises a nucleotide sequence corresponding to the cDNA sequence of SEQ ID NO:150 from nucleotide 1 to nucleotide 423, and extending contiguously from a nucleotide sequence corresponding to the 5′ end of said sequence of SEQ ID NO:150 from nucleotide 1 to nucleotide 423, to a nucleotide sequence corresponding to the 3′ end of said sequence of SEQ ID NO:150 from nucleotide 1 to nucleotide 423.

In other embodiments, the present invention provides a composition comprising a protein, wherein said protein comprises an amino acid sequence selected from the group consisting of:
(a) the amino add sequence of SEQ ID NO:151;
(b) the amino acid sequence of SEQ ID NO:151 from amino acid 1 to amino acid 104;
(c) a fragment of the amino acid sequence of SEQ ID NO:151, the fragment comprising eight contiguous amino acids of SEQ ID NO:151; and
(d) the amino acid sequence encoded by the cDNA insert of clone C1585_{—}1 deposited with the ATCC under accession number 98278;
   the protein being substantially free from other mammalian proteins. Preferably such protein comprises the amino acid sequence of SEQ ID NO:151 or the amino acid sequence of SEQ ID NO:151 from amino acid 1 to amino acid 104. In further preferred embodiments, the present invention provides a protein comprising a fragment of the amino acid sequence of SEQ ID NO:151 having biological activity, the fragment preferably comprising eight (more preferably twenty, most preferably thirty) contiguous amino acids of SEQ ID NO:151, or a protein comprising a fragment of the amino acid sequence of SEQ ID NO:151 having biological activity, the fragment comprising the amino acid sequence from amino acid 138 to amino acid 147 of SEQ ID NO:151.

In one embodiment, the present invention provides a composition comprising an isolated polynucleotide selected from the group consisting of:
(a) a polynucleotide comprising the nucleotide sequence of SEQ ID NO:152;
(b) a polynucleotide comprising the nucleotide sequence of SEQ ID NO:152 from nucleotide 37 to nucleotide 2766;
(c) a polynucleotide comprising the nucleotide sequence of SEQ ID NO:152 from nucleotide 243 to nucleotide 789;
(d) a polynucleotide comprising the nucleotide sequence of the full-length protein coding sequence of clone CT97_{—}3 deposited with the ATCC under accession number 98278;
(e) a polynucleotide encoding the full-length protein encoded by the cDNA insert of clone CT797_{—}3 deposited with the ATCC under accession number 98278;
(f) a polynucleotide comprising the nucleotide sequence of a mature protein coding sequence of clone CT797_{—}3 deposited with the ATCC under accession number 98278;
(g) a polynucleotide encoding a mature protein encoded by the cDNA insert of clone CT797_{—}3 deposited with the ATCC under accession number 98278;
(h) a polynucleotide encoding a protein comprising the amino acid sequence of SEQ ID NO:153;
(i) a polynucleotide encoding a protein comprising a fragment of the amino acid sequence of SEQ ID NO:153 having biological activity, the fragment comprising eight contiguous amino acids of SEQ ID NO:153;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:152.

Preferably, such polynucleotide comprises the nucleotide sequence of SEQ ID NO:152 from nucleotide 37 to nucleotide 2766; the nucleotide sequence of SEQ ID NO:152 from nucleotide 243 to nucleotide 789; the nucleotide sequence of the full-length protein coding sequence of clone CT797_{—}3 deposited with the ATCC under accession number 98278; or the nucleotide sequence of a mature protein coding sequence of clone CT797_{—}3 deposited with the ATCC under accession number 98278. In other preferred embodiments, the polynucleotide encodes the full-length or a mature protein encoded by the cDNA insert of clone CT797_{—}3 deposited with the ATCC under accession number 98278. In yet other preferred embodiments, the present invention provides a polynucleotide encoding a protein comprising the amino acid sequence of SEQ ID NO:153 from amino acid 75 to amino acid 251. In further preferred embodiments, the present invention provides a polynucleotide encoding a protein comprising a fragment of the amino acid sequence of SEQ ID NO 153 having biological activity, the fragment preferably comprising eight (more preferably twenty, most preferably thirty) contiguous amino acids of SEQ ID NO:153, or a polynucleotide encoding a protein comprising a fragment of the amino acid sequence of SEQ ID NO:153 having biological activity, the fragment comprising the amino acid sequence from amino acid 450 to amino acid 459 of SEQ ID NO:153.

Other embodiments provide the gene corresponding to the cDNA sequence of SEQ ID NO:152.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:152, but excluding the poly(A) tail at the 3′ end of SEQ ID NO:152; and
      (ab) the nucleotide sequence of the cDNA insert of clone C1797_{—}3 deposited with the ATCC under accession number 98278;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:152, but excluding the poly(A) tail at the 3′ end of SEQ ID NO:152; and
      (bb) the nucleotide sequence of the cDNA insert of clone CT797_{—}3 deposited with the ATCC under accession number 98278;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:152, and extending contiguously from a nucleotide sequence corresponding to the 5′ end of SEQ ID NO:152 to a nucleotide sequence corresponding to the 3′ end of SEQ ID NO:152, but excluding the poly(A) tail at the 3′ end of SEQ ID NO:152. Also preferably the polynucleotide isolated according to the above process comprises a nucleotide sequence corresponding to the cDNA sequence of SEQ ID NO:152 from nucleotide 37 to nucleotide 2766, and extending contiguously from a nucleotide sequence corresponding to the 5′ end of said sequence of SEQ ID NO:152 from nucleotide 37 to nucleotide 2766, to a nucleotide sequence corresponding to the 3′ end of said sequence of SEQ ID NO:152 from nucleotide 37 to nucleotide 2766. Also preferably the polynucleotide isolated according to the above process comprises a nucleotide sequence corresponding to the cDNA sequence of SEQ ID NO:152 from nucleotide 243 to nucleotide 789, and extending contiguously from a nucleotide sequence corresponding to the 5′ end of said sequence of SEQ ID NO:152 from nucleotide 243 to nucleotide 789, to a nucleotide sequence corresponding to the 3′ end of said sequence of SEQ ID NO:152 from nucleotide 243 to nucleotide 789.

In other embodiments, the present invention provides a composition comprising a protein, wherein said protein comprises an amino acid sequence selected from the group consisting of:
(a) the amino acid sequence of SEQ ID NO:153;
(b) the amino acid sequence of SEQ ID NO:153 from amino add 75 to amino acid 251;
(c) a fragment of the amino add sequence of SEQ ID NO:153, the fragment comprising eight contiguous amino acids of SEQ ID NO:153; and
(d) the amino acid sequence encoded by the cDNA insert of clone CT797_{—}3 deposited with the ATCC under accession number 98278;
   the protein being substantially free from other mammalian proteins. Preferably such protein comprises the amino acid sequence of SEQ ID NO:153 or the amino acid sequence of SEQ ID NO:153 from amino acid 75 to amino acid 251. In further preferred embodiments, the present invention provides a protein comprising a fragment of the amino acid sequence of SEQ ID NO:153 having biological activity, the fragment preferably comprising eight (more preferably twenty, most preferably thirty) contiguous amino acids of SEQ ID NO:153, or a protein comprising a fragment of the amino acid sequence of SEQ ID NO:153 having biological activity, the fragment comprising the amino acid sequence from amino acid 450 to amino acid 459 of SEQ ID NO:153.

In one embodiment, the present invention provides a composition comprising an isolated polynucleotide selected from the group consisting of:
(a) a polynucleotide comprising the nucleotide sequence of SEQ ID NO:155;
(b) a polynucleotide comprising the nucleotide sequence of SEQ ID NO:155 from nucleotide 41 to nucleotide 760;
(c) a polynucleotide comprising the nucleotide sequence of the full-length protein coding sequence of clone CB107_{—}1 deposited with the ATCC under accession number 98279;
(d) a polynucleotide encoding the full-length protein encoded by the cDNA insert of clone CB107_deposited with the ATCC under accession number 98279;
(e) a polynucleotide comprising the nucleotide sequence of a mature protein coding sequence of clone CB107_{—}1 deposited with the ATCC under accession number 98279;
(f) a polynucleotide encoding a mature protein encoded by the cDNA insert of clone CB107_{—}1 deposited with the ATCC under accession number 98279;
(g) a polynucleotide encoding a protein comprising the amino acid sequence of SEQ ID NO:156;
(h) a polynucleotide encoding a protein comprising a fragment of the amino acid sequence of SEQ ID NO:156 having biological activity, the fragment comprising eight contiguous amino acids of SEQ ID NO:156;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:155.

Preferably, such polynucleotide comprises the nucleotide sequence of SEQ ID NO:155 from nucleotide 41 to nucleotide 760; the nucleotide sequence of the full-length protein coding sequence of clone CB107_{—}1 deposited with the ATCC under accession number 98279; or the nucleotide sequence of a mature protein coding sequence of clone CB107_{—}1 deposited with the ATCC under accession number 98279. In other preferred embodiments, the polynucleotide encodes the full-length or a mature protein encoded by the cDNA insert of clone CB107_{—}1 deposited with the ATCC under accession number 98279. In yet other preferred embodiments, the present invention provides a polynucleotide encoding a protein comprising the amino add sequence of SEQ ID NO:156 from amino acid 127 to amino acid 240. In further preferred embodiments, the present invention provides a polynucleotide encoding a protein comprising a fragment of the amino acid sequence of SEQ ID NO:156 having biological activity, the fragment preferably comprising eight (more preferably twenty, most preferably thirty) contiguous amino acids of SEQ ID NO:156, or a polynucleotide encoding a protein comprising a fragment of the amino acid sequence of SEQ ID NO:156 having biological activity, the fragment comprising the amino acid sequence from amino acid 115 to amino acid 124 of SEQ ID NO:156.

Other embodiments provide the gene corresponding to the cDNA sequence of SEQ ID NO:155, SEQ ID NO:154, and SEQ ID NO:157.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:154;
      (ab) SEQ ID NO:155;
      (ac) SEQ ID NO:157, but excluding the poly(A) tail at the 3′ end of SEQ ID NO:157; and
      (ad) the nucleotide sequence of the cDNA insert of clone CB107_deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:154;
      (bb) SEQ ID NO:155;
      (bc) SEQ ID NO:157, but excluding the poly(A) tail at the 3′ end of SEQ ID NO:157; and
      (bd) the nucleotide sequence of the cDNA insert of clone CB107_{—}1 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:154, SEQ ID NO:155, and SEQ ID. NO:157, and extending contiguously from a nucleotide sequence corresponding to the 5′ end of SEQ ID NO:154 to a nucleotide sequence corresponding to the 3′ end of SEQ ID NO:157, but excluding the poly(A) tail at the 3′ end of SEQ ID NO:157. Also preferably the polynucleotide isolated according to the above process comprises a nucleotide sequence corresponding to the cDNA sequence of SEQ ID NO:155, and extending contiguously from a nucleotide sequence corresponding to the 5′ end of SEQ ID NO:155 to a nucleotide sequence corresponding to the 3′ end of SEQ ID NO:155. Also preferably the polynucleotide isolated according to the above process comprises a nucleotide sequence corresponding to the cDNA sequence of SEQ ID NO:155 from nucleotide 41 to nucleotide 760, and extending contiguously from a nucleotide sequence corresponding to the 5′ end of said sequence of SEQ ID NO:155 from nucleotide 41 to nucleotide 760, to a nucleotide sequence corresponding to the 3′ end of said sequence of SEQ ID NO:155 from nucleotide 41 to nucleotide 760.

In other embodiments, the present invention provides a composition comprising a protein, wherein said protein comprises an amino acid sequence selected from the group consisting of:
(a) the amino acid sequence of SEQ ID NO:156;
(b) the amino add sequence of SEQ ID NO:156 from amino acid 127 to amino acid 240;
(c) a fragment of the amino acid sequence of SEQ ID NO:156, the fragment comprising eight contiguous amino acids of SEQ ID NO:156; and
(d) the amino acid sequence encoded by the cDNA insert of clone CB107_{—}1 deposited with the ATCC under accession number 98279;
   the protein being substantially free from other mammalian proteins. Preferably such protein comprises the amino acid sequence of SEQ ID NO:156 or the amino acid sequence of SEQ ID NO:156 from amino acid 127 to amino acid 240. In further preferred embodiments, the present invention provides a protein comprising a fragment of the amino acid sequence of SEQ ID NO:156 having biological activity, the fragment preferably comprising eight (more preferably twenty, most preferably thirty) contiguous amino acids of SEQ ID NO:156, or a protein comprising a fragment of the amino acid sequence of SEQ ID NO:156 having biological activity, the fragment comprising the amino add sequence from amino acid 115 to amino acid 124 of SEQ ID NO:156.

In one embodiment, the present invention provides a composition comprising an isolated polynucleotide selected from the group consisting of:
(a) a polynucleotide comprising the nucleotide sequence of SEQ ID NO:158;
(b) a polynucleotide comprising the nucleotide sequence of SEQ ID NO:158 from nucleotide 374 to nucleotide 1108;
(c) a polynucleotide comprising the nucleotide sequence of SEQ ID NO:158 from nucleotide 500 to nucleotide 1108;
(d) a polynucleotide comprising the nucleotide sequence of SEQ ID NO:158 from nucleotide 1 to nucleotide 387;
(e) a polynucleotide comprising the nucleotide sequence of the full-length protein coding sequence of clone CG300_{—}3 deposited with the ATCC under accession number 98279;
(f) a polynucleotide encoding the full-length protein encoded by the cDNA insert of clone CG300_{—}3 deposited with the ATCC under accession number 98279;
(g) a polynucleotide comprising the nucleotide sequence of a mature protein coding sequence of clone CG300_{—}3 deposited with the ATCC under accession number 98279;
(h) a polynucleotide encoding a mature protein encoded by the cDNA insert of clone CG300_{—}3 deposited with the ATCC under accession number 98279;
(i) a polynucleotide encoding a protein comprising the amino acid sequence of SEQ ID NO:159;
(j) a polynucleotide encoding a protein comprising a fragment of the amino acid sequence of SEQ ID NO:159 having biological activity, the fragment comprising eight contiguous amino acids of SEQ ID NO:159;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:158.

Preferably, such polynucleotide comprises the nucleotide sequence of SEQ ID NO:158 from nucleotide 374 to nucleotide 1108; the nucleotide sequence of SEQ ID NO:158 from nucleotide 500 to nucleotide 1108; the nucleotide sequence of SEQ ID NO:158 from nucleotide 1 to nucleotide 387; the nucleotide sequence of the full-length protein coding sequence of clone CG300_{—}3 deposited with the ATCC under accession number 98279; or the nucleotide sequence of a mature protein coding sequence of clone CG300_{—}3 deposited with the ATCC under accession number 98279. In other preferred embodiments, the polynucleotide encodes the full-length or a mature protein encoded by the cDNA insert of clone CG300_{—}3 deposited with the ATCC under accession number 98279. In yet other preferred embodiments, the present invention provides a polynucleotide encoding a protein comprising the amino acid sequence of SEQ ID NO:159 from amino acid 23 to amino acid 57. In further preferred embodiments, the present invention provides a polynucleotide encoding a protein comprising a fragment of the amino acid sequence of SEQ ID NO:159 having biological activity, the fragment preferably comprising eight (more preferably twenty, most preferably thirty) contiguous amino acids of SEQ ID NO:159, or a polynucleotide encoding a protein comprising a fragment of the amino acid sequence of SEQ ID NO:159 having biological activity, the fragment comprising the amino acid sequence from amino acid 117 to amino acid 126 of SEQ ID NO:159.

Other embodiments provide the gene corresponding to the cDNA sequence of SEQ ID NO:158.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:158, but excluding the poly(A) tail at the 3′ end of SEQ ID NO:158; and
      (ab) the nucleotide sequence of the cDNA insert of clone CG300_{—}3 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:158, but excluding the poly(A) tail at the 3′ end of SEQ ID NO:158; and
      (bb) the nucleotide sequence of the cDNA insert of clone CG300_{—}3 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:158, and extending contiguously from a nucleotide sequence corresponding to the 5′ end of SEQ ID NO:158 to a nucleotide sequence corresponding to the 3′ end of SEQ ID NO:158, but excluding the poly(A) tail at the 3′ end of SEQ ID NO:158. Also preferably the polynucleotide isolated according to the above process comprises a nucleotide sequence corresponding to the cDNA sequence of SEQ ID NO:158 from nucleotide 374 to nucleotide 1108, and extending contiguously from a nucleotide sequence corresponding to the 5′ end of said sequence of SEQ ID NO:158 from nucleotide 374 to nucleotide 1108, to a nucleotide sequence corresponding to the 3′ end of said sequence of SEQ ID NO:158 from nucleotide 374 to nucleotide 1108. Also preferably the polynucleotide isolated according to the above process comprises a nucleotide sequence corresponding to the cDNA sequence of SEQ ID NO:158 from nucleotide 500 to nucleotide 1108, and extending contiguously from a nucleotide sequence corresponding to the 5′ end of said sequence of SEQ ID NO:158 from nucleotide 500 to nucleotide 1108, to a nucleotide sequence corresponding to the 3′ end of said sequence of SEQ ID NO:158 from nucleotide 500 to nucleotide 1108. Also preferably the polynucleotide isolated according to the above process comprises a nucleotide sequence corresponding to the cDNA sequence of SEQ ID NO:158 from nucleotide 1 to nucleotide 387, and extending contiguously from a nucleotide sequence corresponding to the 5′ end of said sequence of SEQ ID NO:158 from nucleotide 1 to nucleotide 387, to a nucleotide sequence corresponding to the 3′ end of said sequence of SEQ ID NO 158 from nucleotide 1 to nucleotide 387.

In other embodiments, the present invention provides a composition comprising a protein, wherein said protein comprises an amino acid sequence selected from the group consisting of:
(a) the amino acid sequence of SEQ ID NO:159;
(b) the amino acid sequence of SEQ ID NO:159 from amino acid 23 to amino acid 57;
(c) a fragment of the amino acid sequence of SEQ ID NO:159, the fragment comprising eight contiguous amino acids of SEQ ID NO:159; and
(d) the amino acid sequence encoded by the cDNA insert of clone CG300_{—}3 deposited with the ATCC under accession number 98279;
   the protein being substantially free from other mammalian proteins. Preferably such protein comprises the amino acid sequence of SEQ ID NO:159 or the amino acid sequence of SEQ ID NO:159 from amino acid 23 to amino acid 57. In further preferred embodiments, the present invention provides a protein comprising a fragment of the amino acid sequence of SEQ ID NO:159 having biological activity, the fragment preferably comprising eight (more preferably twenty, most preferably thirty) contiguous amino acids of SEQ ID NO 159, or a protein comprising a fragment of the amino acid sequence of SEQ ID NO:159 having biological activity, the fragment comprising the amino acid sequence from amino acid 117 to amino acid 126 of SEQ ID NO:159.

In one embodiment, the present invention provides a composition comprising an isolated polynucleotide selected from the group consisting of:
(a) a polynucleotide comprising the nucleotide sequence of SEQ ID NO:160;
(b) a polynucleotide comprising the nucleotide sequence of SEQ ID NO:160 from nucleotide 126 to nucleotide 3053;
(c) a polynucleotide comprising the nucleotide sequence of SEQ ID NO:160 from nucleotide 180 to nucleotide 3053;
(d) a polynucleotide comprising the nucleotide sequence of SEQ ID NO:160 from nucleotide 49 to nucleotide 382;
(e) a polynucleotide comprising the nucleotide sequence of the full-length protein coding sequence of clone CJ145_{—}1 deposited with the ATCC under accession number 98279;
(f) a polynucleotide encoding the full-length protein encoded by the cDNA insert of clone CJ145_{—}1 deposited with the ATCC under accession number 98279;
(g) a polynucleotide comprising the nucleotide sequence of a mature protein coding sequence of clone CJ145_{—}1 deposited with the ATCC under accession number 98279;
(h) a polynucleotide encoding a mature protein encoded by the cDNA insert of clone CJ145_{—}1 deposited with the ATCC under accession number 98279;
(i) a polynucleotide encoding a protein comprising the amino acid sequence of SEQ ID NO:161;
(j) a polynucleotide encoding a protein comprising a fragment of the amino acid sequence of SEQ ID NO:161 having biological activity, the fragment comprising eight contiguous amino adds of SEQ ID NO:161;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:160.

Preferably, such polynucleotide comprises the nucleotide sequence of SEQ ID NO:160 from nucleotide 126 to nucleotide 3053; the nucleotide sequence of SEQ ID NO:160 from nucleotide 180 to nucleotide 3053; the nucleotide sequence of SEQ ID NO:160 from nucleotide 49 to nucleotide 382; the nucleotide sequence of the fill-length protein coding sequence of clone CJ145_{—}1 deposited with the ATCC under accession number 98279; or the nucleotide sequence of a mature protein coding sequence of clone CJ145_{—}1 deposited with the ATCC under accession number 98279. In other preferred embodiments, the polynucleotide encodes the full-length or a mature protein encoded by the cDNA insert of clone CJ145_{—}1 deposited with the ATCC under accession number 98279. In yet other preferred embodiments, the present invention provides a polynucleotide encoding a protein comprising the amino acid sequence of SEQ ID NO:161 from amino acid 1 to amino acid 87. In further preferred embodiments, the present invention provides a polynucleotide encoding a protein comprising a fragment of the amino acid sequence of SEQ ID NO:161 having biological activity, the fragment preferably comprising eight (more preferably twenty, most preferably thirty) contiguous amino acids of SEQ ID NO:161, or a polynucleotide encoding a protein comprising a fragment of the amino acid sequence of SEQ ID NO:161 having biological activity, the fragment comprising the amino acid sequence from amino acid 482 to amino acid 491 of SEQ ID NO:161.

Other embodiments provide the gene corresponding to the cDNA sequence of SEQ ID NO:160.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:160, but excluding the poly(A) tail at the 3′ end of SEQ ID NO:160; and
      (ab) the nucleotide sequence of the cDNA insert of clone Q145_{—}1 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:160, but excluding the poly(A) tail at the 3′ end of SEQ ID NO:160; and
      (bb) the nucleotide sequence of the cDNA insert of clone CJ145_{—}1 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
(iv) isolating the polynucleotide products of step (b)(iii).
   Preferably the polynucleotide isolated according to the above process comprises a nucleotide sequence corresponding to the cDNA sequence of SEQ ID NO:160, and extending contiguously from a nucleotide sequence corresponding to the 5′ end of SEQ ID NO:160 to a nucleotide sequence corresponding to the 3′ end of SEQ ID NO:160, but excluding the poly(A) tail at the 3′ end of SEQ ID NO:160. Also preferably the polynucleotide isolated according to the above process comprises a nucleotide sequence corresponding to the cDNA sequence of SEQ ID NO:160 from nucleotide 126 to nucleotide 3053, and extending contiguously from a nucleotide sequence corresponding to the 5′ end of said sequence of SEQ ID NO:160 from nucleotide 126 to nucleotide 3053, to a nucleotide sequence corresponding to the 3′ end of said sequence of SEQ ID NO:160 from nucleotide 126 to nucleotide 3053. Also preferably the polynucleotide isolated according to the above process comprises a nucleotide sequence corresponding to the cDNA sequence of SEQ ID NO:160 from nucleotide 180 to nucleotide 3053, and extending contiguously from a nucleotide sequence corresponding to the 5′ end of said sequence of SEQ ID NO:160 from nucleotide 180 to nucleotide 3053, to a nucleotide sequence corresponding to the 3′ end of said sequence of SEQ ID NO:160 from nucleotide 180 to nucleotide 3053. Also preferably the polynucleotide isolated according to the above process comprises a nucleotide sequence corresponding to the cDNA sequence of SEQ ID NO:160 from nucleotide 49 to nucleotide 382, and extending contiguously from a nucleotide sequence corresponding to the 5′ end of said sequence of SEQ ID NO:160 from nucleotide 49 to nucleotide 382, to a nucleotide sequence corresponding to the 3′ end of said sequence of SEQ ID NO:160 from nucleotide 49 to nucleotide 382.

In other embodiments, the present invention provides a composition comprising a protein, wherein said protein comprises an amino acid sequence selected from the group consisting of:
(a) the amino acid sequence of SEQ ID NO:161;
(b) the amino acid sequence of SEQ ID NO:161 from amino acid 1 to amino acid 87;
(c) a fragment of the amino acid sequence of SEQ ID NO:161, the fragment comprising eight contiguous amino acids of SEQ ID NO:161; and
(d) the amino acid sequence encoded by the cDNA insert of clone CJ145_{—}1 deposited with the ATCC under accession number 98279;
   the protein being substantially free from other mammalian proteins. Preferably such protein comprises the amino acid sequence of SEQ ID NO:161 or the amino acid sequence of SEQ ID NO:161 from amino acid 1 to amino acid 87. In further preferred embodiments, the present invention provides a protein comprising a fragment of the amino acid sequence of SEQ ID NO:161 having biological activity, the fragment preferably comprising eight (more preferably twenty, most preferably thirty) contiguous amino acids of SEQ ID NO:161, or a protein comprising a fragment of the amino acid sequence of SEQ ID NO:161 having biological activity, the fragment comprising the amino acid sequence from amino acid 482 to amino acid 491 of SEQ ID NO:161.

In one embodiment, the present invention provides a composition comprising an isolated polynucleotide selected from the group consisting of:
(a) a polynucleotide comprising the nucleotide sequence of SEQ ID NO:162;
(b) a polynucleotide comprising the nucleotide sequence of SEQ ID NO:162 from nucleotide 40 to nucleotide 342;
(c) a polynucleotide comprising the nucleotide sequence of SEQ ID NO:162 from nucleotide 127 to nucleotide 342;
(d) a polynucleotide comprising the nucleotide sequence of SEQ ID NO:162 from nucleotide 11 to nucleotide 181;
(e) a polynucleotide comprising the nucleotide sequence of the full-length protein coding sequence of clone CJ160_{—}11 deposited with the ATCC under accession number 98279;
(f) a polynucleotide encoding the full-length protein encoded by the cDNA insert of clone CJ160_{—}11 deposited with the ATCC under accession number 98279;
(g) a polynucleotide comprising the nucleotide sequence of a mature protein coding sequence of clone CJ160_{—}11 deposited with the ATCC under accession number 98279;
(h) a polynucleotide encoding a mature protein encoded by the cDNA insert of clone CJ160_{—}11 deposited with the ATCC under accession number 98279;
(i) a polynucleotide encoding a protein comprising the amino add sequence of SEQ ID NO:163;
(j) a polynucleotide encoding a protein comprising a fragment of the amino acid sequence of SEQ ID NO:163 having biological activity, the fragment comprising eight contiguous amino acids of SEQ ID NO:163;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:162.

Preferably, such polynucleotide comprises the nucleotide sequence of SEQ ID NO:162 from nucleotide 40 to nucleotide 342; the nucleotide sequence of SEQ ID NO:162 from nucleotide 127 to nucleotide 342; the nucleotide sequence of SEQ ID NO:162 from nucleotide 11 to nucleotide 181; the nucleotide sequence of the full-length protein coding sequence of clone CJ160_{—}11 deposited with the ATCC under accession number 98279; or the nucleotide sequence of a mature protein coding sequence of clone CJ160_{—}11 deposited with the ATCC under accession number 98279. In other preferred embodiments, the polynucleotide encodes the full-length or a mature protein encoded by the cDNA insert of clone CJ160_{—}11 deposited with the ATCC under accession number 98279. In yet other preferred embodiments, the present invention provides a polynucleotide encoding a protein comprising the amino acid sequence of SEQ ID NO:163 from amino acid 7 to amino acid 48. In further preferred embodiments, the present invention provides a polynucleotide encoding a protein comprising a fragment of the amino acid sequence of SEQ ID NO:163 having biological activity, the fragment preferably comprising eight (more preferably twenty, most preferably thirty) contiguous amino acids of SEQ ID NO:163, or a polynucleotide encoding a protein comprising a fragment of the amino acid sequence of SEQ ID NO:163 having biological activity, the fragment comprising the amino acid sequence from amino acid 45 to amino acid 54 of SEQ ID NO:163.

Other embodiments provide the gene corresponding to the cDNA sequence of SEQ ID NO:162.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:162, but excluding the poly(A) tail at the 3′ end of SEQ ID NO:162; and
      (ab) the nucleotide sequence of the cDNA insert of clone CJ160_{—}11 deposited with the ATCC under accession number 98279;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:162, but excluding the poly(A) tail at the 3′ end of SEQ ID NO:162; and
      (bb) the nucleotide sequence of the cDNA insert of clone CJ160_{—}11 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:162, and extending contiguously from a nucleotide sequence corresponding to the 5′ end of SEQ ID NO:162 to a nucleotide sequence corresponding to the 3′ end of SEQ ID NO:162, but excluding the poly(A) tail at the 3′ end of SEQ ID NO:162. Also preferably the polynucleotide isolated according to the above process comprises a nucleotide sequence corresponding to the cDNA sequence of SEQ ID NO:162 from nucleotide 40 to nucleotide 342, and extending contiguously from a nucleotide sequence corresponding to the 5′ end of said sequence of SEQ ID NO:162 from nucleotide 40 to nucleotide 342, to a nucleotide sequence corresponding to the 3′ end of said sequence of SEQ ID NO:162 from nucleotide 40 to nucleotide 342. Also preferably the polynucleotide isolated according to the above process comprises a nucleotide sequence corresponding to the cDNA sequence of SEQ ID NO:162 from nucleotide 127 to nucleotide 342, and extending contiguously from a nucleotide sequence corresponding to the 5′ end of said sequence of SEQ ID NO:162 from nucleotide 127 to nucleotide 342, to a nucleotide sequence corresponding to the 3′ end of said sequence of SEQ ID NO:162 from nucleotide 127 to nucleotide 342. Also preferably the polynucleotide isolated according to the above process comprises a nucleotide sequence corresponding to the cDNA sequence of SEQ ID NO:162 from nucleotide 11 to nucleotide 181, and extending contiguously from a nucleotide sequence corresponding to the 5′ end of said sequence of SEQ ID NO:162 from nucleotide 11 to nucleotide 181, to a nucleotide sequence corresponding to the 3′ end of said sequence of SEQ ID NO:162 from nucleotide 11 to nucleotide 181.

In other embodiments, the present invention provides a composition comprising a protein, wherein said protein comprises an amino acid sequence selected from the group consisting of:
(a) the amino acid sequence of SEQ ID NO:163;
(b) the amino acid sequence of SEQ ID NO:163 from amino acid 7 to amino acid 48;
(c) a fragment of the amino acid sequence of SEQ ID NO:163, the fragment comprising eight contiguous amino acids of SEQ ID NO:163; and
(d) the amino acid sequence encoded by the cDNA insert of clone CJ160_{—}11 deposited with the ATCC under accession number 98279;
   the protein being substantially free from other mammalian proteins. Preferably such protein comprises the amino acid sequence of SEQ ID NO:163 or the amino acid sequence of SEQ ID NO:163 from amino acid 7 to amino acid 48. In further preferred embodiments, the present invention provides a protein comprising a fragment of the amino acid sequence of SEQ ID NO:163 having biological activity, the fragment preferably comprising eight (more preferably twenty, most preferably thirty) contiguous amino acids of SEQ ID NO:163, or a protein comprising a fragment of the amino acid sequence of SEQ ID NO:163 having biological activity, the fragment comprising the amino acid sequence from amino acid 45 to amino acid 54 of SEQ ID NO:163.

In one embodiment, the present invention provides a composition comprising an isolated polynucleotide selected from the group consisting of:
(a) a polynucleotide comprising the nucleotide sequence of SEQ ID NO:164;
(b) a polynucleotide comprising the nucleotide sequence of SEQ ID NO:164 from nucleotide 180 to nucleotide 467;
(c) a polynucleotide comprising the nucleotide sequence of SEQ ID NO:164 from nucleotide 267 to nucleotide 467;
(d) a polynucleotide comprising the nucleotide sequence of the full-length protein coding sequence of clone CO20_{—}1 deposited with the ATCC under accession number 98279;
(e) a polynucleotide encoding the full-length protein encoded by the cDNA insert of clone CO20_{—}1 deposited with the ATCC under accession number 98279;
(f) a polynucleotide comprising the nucleotide sequence of a mature protein coding sequence of clone CO20_{—}1 deposited with the ATCC under accession number 98279;
(g) a polynucleotide encoding a mature protein encoded by the cDNA insert of clone CO20_{—}1 deposited with the ATCC under accession number 98279;
(h) a polynucleotide encoding a protein comprising the amino acid sequence of SEQ ID NO:165;
(i) a polynucleotide encoding a protein comprising a fragment of the amino acid sequence of SEQ ID NO:165 having biological activity, the fragment comprising eight contiguous amino acids of SEQ ID NO:165;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:164.

Preferably, such polynucleotide comprises the nucleotide sequence of SEQ ID NO:164 from nucleotide 180 to nucleotide 467; the nucleotide sequence of SEQ ID NO:164 from nucleotide 267 to nucleotide 467; the nucleotide sequence of the full-length protein coding sequence of clone CO20_{—}1 deposited with the ATCC under accession number 98279; or the nucleotide sequence of a mature protein coding sequence of clone CO20_{—}1 deposited with the. ATCC under accession number 98279. In other preferred embodiments, the polynucleotide encodes the full-length or a mature protein encoded by the cDNA insert of clone CO20_{—}1 deposited with the ATCC under accession number 98279. In yet other preferred embodiments, the present invention provides a polynucleotide encoding a protein comprising the amino acid sequence of SEQ ID NO:165 from amino acid 1 to amino acid 37. In further preferred embodiments, the present invention provides a polynucleotide encoding a protein comprising a fragment of the amino acid sequence of SEQ ID NO:165 having biological activity, the fragment preferably comprising eight (more preferably twenty, most preferably thirty) contiguous amino acids of SEQ ID NO:165, or a polynucleotide encoding a protein comprising a fragment of the amino add sequence of SEQ ID NO:165 having biological activity, the fragment comprising the amino acid sequence from amino acid 43 to amino acid 52 of SEQ ID NO:165.

Other embodiments provide the gene corresponding to the cDNA sequence of SEQ ID NO:164 and SEQ ID NO:166.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:164;
      (ab) SEQ ID NO:166, but excluding the poly(A) tail at the 3′ end of SEQ ID NO:166; and
      (ac) the nucleotide sequence of the cDNA insert of clone CO20_{—}1 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:164;
      (bb) SEQ ID NO:166, but excluding the poly(A) tail at the 3′ end of SEQ ID NO:166; and
      (bc) the nucleotide sequence of the cDNA insert of clone CO20_{—}1 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
(iv) isolating the polynucleotide products of step (b)(iii).
   Preferably the polynucleotide isolated according to the above process comprises a nucleotide sequence corresponding to the cDNA sequences of SEQ ID NO:164 and SEQ ID NO:166, and extending contiguously from a nucleotide sequence corresponding to the 5′ end of SEQ ID NO:164 to a nucleotide sequence corresponding to the 3′ end of SEQ ID NO:166, but excluding the poly(A) tail at the 3′ end of SEQ ID NO:166. Also preferably the polynucleotide isolated according to the above process comprises a nucleotide sequence corresponding to the cDNA sequence of SEQ ID NO:164, and extending contiguously from a nucleotide sequence corresponding to the 5′ end of SEQ ID NO:164 to a nucleotide sequence corresponding to the 3′ end of SEQ ID NO:164. Also preferably the polynucleotide isolated according to the above process comprises a nucleotide sequence corresponding to the cDNA sequence of SEQ ID NO:164 from nucleotide 180 to nucleotide 467, and extending contiguously from a nucleotide sequence corresponding to the 5′ end of said sequence of SEQ ID NO:164 from nucleotide 180 to nucleotide 467, to a nucleotide sequence corresponding to the 3′ end of said sequence of SEQ ID NO:164 from nucleotide 180 to nucleotide 467. Also preferably the polynucleotide isolated according to the above process comprises a nucleotide sequence corresponding to the cDNA sequence of SEQ ID NO:164 from nucleotide 267 to nucleotide 467, and extending contiguously from a nucleotide sequence corresponding to the 5′ end of said sequence of SEQ ID NO:164 from nucleotide 267 to nucleotide 467, to a nucleotide sequence corresponding to the 3′ end of said sequence of SEQ ID NO:164 from nucleotide 267 to nucleotide 467.

In other embodiments, the present invention provides a composition comprising a protein, wherein said protein comprises an amino add sequence selected from the group consisting of:
(a) the amino acid sequence of SEQ ID NO:165;
(b) the amino acid sequence of SEQ ID NO:165 from amino acid 1 to amino acid 37;
(c) a fragment of the amino acid sequence of SEQ ID NO:165, the fragment comprising eight contiguous amino adds of SEQ ID NO:165; and
(d) the amino acid sequence encoded by the cDNA insert of clone CO20_{—}1 deposited with the ATCC under accession number 98279; the protein being substantially free from other mammalian proteins. Preferably such protein comprises the amino add sequence of SEQ ID NO:165 or the amino acid sequence of SEQ ID NO:165 from amino add 1 to amino acid 37. In further preferred embodiments, the present invention provides a protein comprising a fragment of the amino acid sequence of SEQ ID NO:165 having biological activity, the fragment preferably comprising eight (more preferably twenty, most preferably thirty) contiguous amino adds of SEQ ID NO:165, or a protein comprising a fragment of the amino add sequence of SEQ ID NO:165 having biological activity, the fragment comprising the amino acid sequence from amino acid 43 to amino acid 52 of SEQ ID NO:165.

In one embodiment, the present invention provides a composition comprising an isolated polynucleotide selected from the group consisting of:
(a) a polynucleotide comprising the nucleotide sequence of SEQ ID NO:167;
(b) a polynucleotide comprising the nucleotide sequence of SEQ ID NO:167 from nucleotide 176 to nucleotide 520;
(c) a polynucleotide comprising the nucleotide sequence of SEQ ID NO:167 from nucleotide 317 to nucleotide 520;
(d) a polynucleotide comprising the nucleotide sequence of SEQ ID NO:167 from nucleotide 118 to nucleotide 413;
(e) a polynucleotide comprising the nucleotide sequence of the full-length protein coding sequence of clone CO223_{—}3 deposited with the ATCC under accession number 98291;
(f) a polynucleotide encoding the full-length protein encoded by the cDNA insert of clone CO223_{—}3 deposited with the ATCC under accession number 98291;
(g) a polynucleotide comprising the nucleotide sequence of a mature protein coding sequence of clone CO223_{—}3 deposited with the ATCC under accession number 98291;
(h) a polynucleotide encoding a mature protein encoded by the cDNA insert of clone CO223_{—}3 deposited with the ATCC under accession number 98291;
(i) a polynucleotide encoding a protein comprising the amino acid sequence of SEQ ID NO:168;
(j) a polynucleotide encoding a protein comprising a fragment of the amino acid sequence of SEQ ID NO:168 having biological activity, the fragment comprising eight contiguous amino acids of SEQ ID NO:168;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:167.

Preferably, such polynucleotide comprises the nucleotide sequence of SEQ ID NO:167 from nucleotide 176 to nucleotide 520; the nucleotide sequence of SEQ ID NO:167 from nucleotide 317 to nucleotide 520; the nucleotide sequence of SEQ ID NO:167 from nucleotide 118 to nucleotide 413; the nucleotide sequence of the full-length protein coding sequence of clone CO223_{—}3 deposited with the ATCC under accession number 98291; or the nucleotide sequence of a mature protein coding sequence of clone CO223_{—}3 deposited with the ATCC under accession number 98291. In other preferred embodiments, the polynucleotide encodes the full-length or a mature protein encoded by the cDNA insert of clone CO223_{—}3 deposited with the ATCC under accession number 98291. In yet other preferred embodiments, the present invention provides a polynucleotide encoding a protein comprising the amino acid sequence of SEQ ID NO:168 from amino add 1 to amino add 80. In further preferred embodiments, the present invention provides a polynucleotide encoding a protein comprising a fragment of the amino acid sequence of SEQ ID NO:168 having biological activity, the fragment preferably comprising eight (more preferably twenty, most preferably thirty) contiguous amino adds of SEQ ID NO:168, or a polynucleotide encoding a protein comprising a fragment of the amino acid sequence of SEQ ID NO:168 having biological activity, the fragment comprising the amino acid sequence from amino add 52 to amino acid 61 of SEQ ID NO:168.

Other embodiments provide the gene corresponding to the cDNA sequence of SEQ ID NO:167.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:167, but excluding the poly(A) tail at the 3′ end of SEQ ID NO:167; and
      (ab) the nucleotide sequence of the cDNA insert of clone CO223_{—}3 deposited with the ATCC under accession number 98291;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:167, but excluding the poly(A) tail at the 3′ end of SEQ. ID NO:167; and
      (bb) the nucleotide sequence of the cDNA insert of clone CO223_{—}3 deposited with the ATCC under accession number 98291;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:167, and extending contiguously from a nucleotide sequence corresponding to the 5′ end of SEQ ID NO:167 to a nucleotide sequence corresponding to the 3′ end of SEQ ID NO:167, but excluding the poly(A) tail at the 3′ end of SEQ ID NO:167. Also preferably the polynucleotide isolated according to the above process comprises a nucleotide sequence corresponding to the cDNA sequence of SEQ ID NO:167 from nucleotide 176 to nucleotide 520, and extending contiguously from a nucleotide sequence corresponding to the 5′ end of said sequence of SEQ ID NO:167 from nucleotide 176 to nucleotide 520, to a nucleotide sequence corresponding to the 3′ end of said sequence of SEQ ID NO:167 from nucleotide 176 to nucleotide 520. Also preferably the polynucleotide isolated according to the above process comprises a nucleotide sequence corresponding to the cDNA sequence of SEQ ID NO:167 from nucleotide 317 to nucleotide 520, and extending contiguously from a nucleotide sequence corresponding to the 5′ end of said sequence of SEQ ID NO:167 from nucleotide 317 to nucleotide 520, to a nucleotide sequence corresponding to the 3′ end of said sequence of SEQ ID NO:167 from nucleotide 317 to nucleotide 520. Also preferably the polynucleotide isolated according to the above process comprises a nucleotide sequence corresponding to the cDNA sequence of SEQ ID NO:167 from nucleotide 118 to nucleotide 413, and extending contiguously from a nucleotide sequence corresponding to the 5′ end of said sequence of SEQ ID NO:167 from nucleotide 118 to nucleotide 413, to a nucleotide sequence corresponding to the 3′ end of said sequence of SEQ ID NO:167 from nucleotide 118 to nucleotide 413.

In other embodiments, the present invention provides a composition comprising a protein, wherein said protein comprises an amino acid sequence selected from the group consisting of:
(a) the amino acid sequence of SEQ ID NO:168;
(b) the amino acid sequence of SEQ ID NO:168 from amino acid 1 to amino acid 80;
(c) a fragment of the amino acid sequence of SEQ ID NO:168, the fragment comprising eight contiguous amino acids of SEQ ID NO:168; and
(d) the amino acid sequence encoded by the cDNA insert of clone CO223_{—}3 deposited with the ATCC under accession number 98291;
   the protein being substantially free from other mammalian proteins. Preferably such protein comprises the amino acid sequence of SEQ ID NO:168 or the amino acid sequence of SEQ ID NO:168 from amino add 1 to amino acid 80. In further preferred embodiments, the present invention provides a protein comprising a fragment of the amino acid sequence of SEQ ID NO:168 having biological activity, the fragment preferably comprising eight (more preferably twenty, most preferably thirty) contiguous amino acids of SEQ ID NO:168, or a protein comprising a fragment of the amino acid sequence of SEQ ID NO:168 having biological activity, the fragment comprising the amino acid sequence from amino acid 52 to amino acid 61 of SEQ ID NO:168.

In one embodiment, the present invention provides a composition comprising an isolated polynucleotide selected from the group consisting of:
(a) a polynucleotide comprising the nucleotide sequence of SEQ ID NO:169;
(b) a polynucleotide comprising the nucleotide sequence of SEQ ID NO:169 from nucleotide 303 to nucleotide 542;
(c) a polynucleotide comprising the nucleotide sequence of SEQ ID NO:169 from nucleotide 1 to nucleotide 435;
(d) a polynucleotide comprising the nucleotide sequence of the full-length protein coding sequence of clone CO310_{—}2 deposited with the ATCC under accession number 98279;
(e) a polynucleotide encoding the full-length protein encoded by the cDNA insert of clone CO310_{—}2 deposited with the ATCC under accession number 98279;
(f) a polynucleotide comprising the nucleotide sequence of a mature protein coding sequence of clone CO310_{—}2 deposited with the ATCC under accession number 98279;
(g) a polynucleotide encoding a mature protein encoded by the cDNA insert of clone CO310_{—}2 deposited with the ATCC under accession number 98279;
(h) a polynucleotide encoding a protein comprising the amino acid sequence of SEQ ID NO:170;
(i) a polynucleotide encoding a protein comprising a fragment of the amino acid sequence of SEQ ID NO:170 having biological activity, the fragment comprising eight contiguous amino adds of SEQ ID NO:170;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:169.

Preferably, such polynucleotide comprises the nucleotide sequence of SEQ ID NO:169 from nucleotide 303 to nucleotide 542; the nucleotide sequence of SEQ ID NO:169 from nucleotide 1 to nucleotide 435; the nucleotide sequence of the full-length protein coding sequence of clone CO310_{—}2 deposited with the ATCC under accession number 98279; or the nucleotide sequence of a mature protein coding sequence of clone CO310_{—}2 deposited with the ATCC under accession number 98279. In other preferred embodiments, the polynucleotide encodes the full-length or a mature protein encoded by the cDNA insert of clone CO310_{—}2 deposited with the ATCC under accession number 98279. In yet other preferred embodiments, the present invention provides a polynucleotide encoding a protein comprising the amino add sequence of SEQ ID NO:170 from amino acid 1 to amino acid 44. In further preferred embodiments, the present invention provides a polynucleotide encoding a protein comprising a fragment of the amino acid sequence of SEQ ID NO:170 having biological activity, the fragment preferably comprising eight (more preferably twenty, most preferably thirty) contiguous amino acids of SEQ ID NO:170, or a polynucleotide encoding a protein comprising a fragment of the amino acid sequence of SEQ ID NO:170 having biological activity, the fragment comprising the amino acid sequence from amino acid 34 to amino acid 43 of SEQ ID NO:170.

Other embodiments provide the gene corresponding to the cDNA sequence of SEQ ID NO:169.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:169, but excluding the poly(A) tail at the 3′ end of SEQ ID NO:169; and
      (ab) the nucleotide sequence of the cDNA insert of clone CO310_{—}2 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:169, but excluding the poly(A) tail at the 3′ end of SEQ ID NO:169; and
      (bb) the nucleotide sequence of the cDNA insert of clone CO310_{—}2 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:169, and extending contiguously from a nucleotide sequence corresponding to the 5′ end of SEQ ID NO:169 to a nucleotide sequence corresponding to the 3′ end of SEQ ID NO:169, but excluding the poly(A) tail at the 3′ end of SEQ ID NO:169. Also preferably the polynucleotide isolated according to the above process comprises a nucleotide sequence corresponding to the cDNA sequence of SEQ ID NO:169 from nucleotide 303 to nucleotide 542, and extending contiguously from a nucleotide sequence corresponding to the 5′ end of said sequence of SEQ ID NO:169 from nucleotide 303 to nucleotide 542, to a nucleotide sequence corresponding to the 3′ end of said sequence of SEQ ID NO:169 from nucleotide 303 to nucleotide 542. Also preferably the polynucleotide isolated according to the above process comprises a nucleotide sequence corresponding to the cDNA sequence of SEQ ID NO:169 from nucleotide 1 to nucleotide 435, and extending contiguously from a nucleotide sequence corresponding to the 5′ end of said sequence of SEQ ID NO:169 from nucleotide 1 to nucleotide 435, to a nucleotide sequence corresponding to the 3′ end of said sequence of SEQ ID NO:169 from nucleotide 1 to nucleotide 435. In other embodiments, the present invention provides a composition comprising a protein, wherein said protein comprises an amino acid sequence selected from the group consisting of:
(a) the amino acid sequence of SEQ ID NO:170;
(b) the amino acid sequence of SEQ ID NO:170 from amino acid 1 to amino acid 44;
(c) a fragment of the amino acid sequence of SEQ ID NO:170, the fragment comprising eight contiguous amino acids of SEQ ID NO:170; and
(d) the amino acid sequence encoded by the cDNA insert of clone CO310_{—}2 deposited with the ATCC under accession number 98279;
   the protein being substantially free from other mammalian proteins. Preferably such protein comprises the amino acid sequence of SEQ ID NO:170 or the amino acid sequence of SEQ ID NO:170 from amino acid 1 to amino acid 44. In further preferred embodiments, the present invention provides a protein comprising a fragment of the amino acid sequence of SEQ ID NO:170 having biological activity, the fragment preferably comprising eight (more preferably twenty, most preferably thirty) contiguous amino acids of SEQ ID NO:170, or a protein comprising a fragment of the amino acid sequence of SEQ ID NO:170 having biological activity, the fragment comprising the amino acid sequence from amino acid 34 to amino acid 43 of SEQ ID NO:170.

In one embodiment, the present invention provides a composition comprising an isolated polynucleotide selected from the group consisting of:
(a) a polynucleotide comprising the nucleotide sequence of SEQ ID NO:171;
(b) a polynucleotide comprising the nucleotide sequence of SEQ ID NO:171 from nucleotide 40 to nucleotide 455;
(c) a polynucleotide comprising the nucleotide sequence of SEQ ID NO:171 from nucleotide 85 to nucleotide 455;
(d) a polynucleotide comprising the nucleotide sequence of SEQ ID NO:171 from nucleotide 265 to nucleotide 515;
(e) a polynucleotide comprising the nucleotide sequence of the full-length protein coding sequence of clone CP258_{—}3 deposited with the ATCC under accession number 98279;
(f) a polynucleotide encoding the full-length protein encoded by the cDNA insert of clone CP258_{—}3 deposited with the ATCC under accession number 98279;
(g) a polynucleotide comprising the nucleotide sequence of a mature protein coding sequence of clone CP258_{—}3 deposited with the ATCC under accession number 98279;
(h) a polynucleotide encoding a mature protein encoded by the cDNA insert of clone CP258_{—}3 deposited with the ATCC under accession number 98279;
(i) a polynucleotide encoding a protein comprising the amino add sequence of SEQ ID NO:172;
(j) a polynucleotide encoding a protein comprising a fragment of the amino acid sequence of SEQ ID NO:172 having biological activity, the fragment comprising eight contiguous amino acids of SEQ ID NO:172;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:171.

Preferably, such polynucleotide comprises the nucleotide sequence of SEQ ID NO:171 from nucleotide 40 to nucleotide 455; the nucleotide sequence of SEQ ID NO:171 from nucleotide 85 to nucleotide 455; the nucleotide sequence of SEQ ID NO:171 from nucleotide 265 to nucleotide 515; the nucleotide sequence of the full-length protein coding sequence of clone CP258_{—}3 deposited with the ATCC under accession number 98279; or the nucleotide sequence of a mature protein coding sequence of clone CP258_{—}3 deposited with the ATCC under accession number 98279. In other preferred embodiments, the polynucleotide encodes the full-length or a mature protein encoded by the cDNA insert of clone CP258_{—}3 deposited with the ATCC under accession number 98279. In yet other preferred embodiments, the present invention provides a polynucleotide encoding a protein comprising the amino acid sequence of SEQ ID NO:172 from amino acid 64 to amino acid 138. In further preferred embodiments, the present invention provides a polynucleotide encoding a protein comprising a fragment of the amino acid sequence of SEQ ID NO:172 having biological activity, the fragment preferably comprising eight (more preferably twenty, most preferably thirty) contiguous amino acids of SEQ ID NO:172, or a polynucleotide encoding a protein comprising a fragment of the amino acid sequence of SEQ ID NO:172 having biological activity, the fragment comprising the amino acid sequence from amino acid 64 to amino acid 73 of SEQ ID NO:172.

Other embodiments provide the gene corresponding to the cDNA sequence of SEQ ID NO:171.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:171, but excluding the poly(A) tail at the 3′ end of SEQ ID NO:171; and
      (ab) the nucleotide sequence of the cDNA insert of clone CP258_{—}3 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:171, but excluding the poly(A) tail at the 3′ end of SEQ ID NO:171; and
      (bb) the nucleotide sequence of the cDNA insert of clone CP258_{—}3 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:171, and extending contiguously from a nucleotide sequence corresponding to the 5′ end of SEQ ID NO:171 to a nucleotide sequence corresponding to the 3′ end of SEQ ID NO:171, but excluding the poly(A) tail at the 3′ end of SEQ ID NO:171. Also preferably the polynucleotide isolated according to the above process comprises a nucleotide sequence corresponding to the cDNA sequence of SEQ ID NO:171 from nucleotide 40 to nucleotide 455, and extending contiguously from a nucleotide sequence corresponding to the 5′ end of said sequence of SEQ ID NO:171 from nucleotide 40 to nucleotide 455, to a nucleotide sequence corresponding to the 3′ end of said sequence of SEQ ID NO:171 from nucleotide 40 to nucleotide 455. Also preferably the polynucleotide isolated according to the above process comprises a nucleotide sequence corresponding to the cDNA sequence of SEQ ID NO:171 from nucleotide 85 to nucleotide 455, and extending contiguously from a nucleotide sequence corresponding to the 5′ end of said sequence of SEQ ID NO:171 from nucleotide 85 to nucleotide 455, to a nucleotide sequence corresponding to the 3′ end of said sequence of SEQ ID NO:171 from nucleotide 85 to nucleotide 455. Also preferably the polynucleotide isolated according to the above process comprises a nucleotide sequence corresponding to the cDNA sequence of SEQ ID NO:171 from nucleotide 265 to nucleotide 515, and extending contiguously from a nucleotide sequence corresponding to the 5′ end of said sequence of SEQ ID NO:171 from nucleotide 265 to nucleotide 515, to a nucleotide sequence corresponding to the 3′ end of said sequence of SEQ ID NO:171 from nucleotide 265 to nucleotide 515.

In other embodiments, the present invention provides a composition comprising a protein, wherein said protein comprises an amino acid sequence selected from the group consisting of:
(a) the amino acid sequence of SEQ ID NO:172;
(b) the amino acid sequence of SEQ ID NO:172 from amino acid 64 to amino acid 138;
(c) a fragment of the amino acid sequence of SEQ ID NO:172, the fragment comprising eight contiguous amino acids of SEQ ID NO:172; and
(d) the amino acid sequence encoded by the cDNA insert of clone CP258_{—}3 deposited with the ATCC under accession number 98279;
   the protein being substantially free from other mammalian proteins. Preferably such protein comprises the amino acid sequence of SEQ ID NO:172 or the amino acid sequence of SEQ ID NO:172 from amino acid 64 to amino acid 138. In further preferred embodiments, the present invention provides a protein comprising a fragment of the amino acid sequence of SEQ ID NO:172 having biological activity, the fragment preferably comprising eight (more preferably twenty, most preferably thirty) contiguous amino acids of SEQ ID NO:172, or a protein comprising a fragment of the amino acid sequence of SEQ ID NO:172 having biological activity, the fragment comprising the amino acid sequence from amino acid 64 to amino acid 73 of SEQ ID NO:172.

In one embodiment, the present invention provides a composition comprising an isolated polynucleotide selected from the group consisting of:
(a) a polynucleotide comprising the nucleotide sequence of SEQ ID NO:173;
(b) a polynucleotide comprising the nucleotide sequence of SEQ ID NO:173 from nucleotide 105 to nucleotide 1007;
(c) a polynucleotide comprising the nucleotide sequence of SEQ ID NO:173 from nucleotide 801 to nucleotide 1007;
(d) a polynucleotide comprising the nucleotide sequence of SEQ ID NO:173 from nucleotide 1 to nucleotide 352;
(e) a polynucleotide comprising the nucleotide sequence of the full-length protein coding sequence of clone CW1155_{—}3 deposited with the ATCC under accession number 98279;
(f) a polynucleotide encoding the full-length protein encoded by the cDNA insert of clone CW1155_{—}3 deposited with the ATCC under accession number 98279;
(g) a polynucleotide comprising the nucleotide sequence of a mature protein coding sequence of clone CW1155_{—}3 deposited with the ATCC under accession number 98279;
(h) a polynucleotide encoding a mature protein encoded by the cDNA insert of clone CW1155_{—}3 deposited with the ATCC under accession number 98279;
(i) a polynucleotide encoding a protein comprising the amino acid sequence of SEQ ID NO:174;
(j) a polynucleotide encoding a protein comprising a fragment of the amino acid sequence of SEQ ID NO:174 having biological activity, the fragment comprising eight contiguous amino acids of SEQ ID NO:174;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:173.

Preferably, such polynucleotide comprises the nucleotide sequence of SEQ ID NO:173 from nucleotide 105 to nucleotide 1007; the nucleotide sequence of SEQ ID NO:173 from nucleotide 801 to nucleotide 1007; the nucleotide sequence of SEQ ID NO:173 from nucleotide 1 to nucleotide 352; the nucleotide sequence of the full-length protein coding sequence of clone CW1155_{—}3 deposited with the ATCC under accession number 98279; or the nucleotide sequence of a mature protein coding sequence of clone CW1155_{—}3 deposited with the ATCC under accession number 98279. In other preferred embodiments, the polynucleotide encodes the full-length or a mature protein encoded by the cDNA insert of clone CW1155_{—}3 deposited with the ATCC under accession number 98279. In yet other preferred embodiments, the present invention provides a polynucleotide encoding a protein comprising the amino acid sequence of SEQ ID NO:174 from amino acid 1 to amino acid 83. In further preferred embodiments, the present invention provides a polynucleotide encoding a protein comprising a fragment of the amino acid sequence of SEQ ID NO:174 having biological activity, the fragment preferably comprising eight (more preferably twenty, most preferably thirty) contiguous amino acids of SEQ ID NO:174, or a polynucleotide encoding a protein comprising a fragment of the amino acid sequence of SEQ ID NO:174 having biological activity, the fragment comprising the amino acid sequence from amino acid 145 to amino acid 154 of SEQ ID NO:174.

Other embodiments provide the gene corresponding to the cDNA sequence of SEQ ID NO:173.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:173, but excluding the poly(A) tail at the 3′ end of SEQ ID NO:173; and
      (ab) the nucleotide sequence of the cDNA insert of clone CW1155_{—}3 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:173, but excluding the poly(A) tail at the 3′ end of SEQ ID NO:173; and
      (bb) the nucleotide sequence of the cDNA insert of clone CW1155_{—}3 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:173, and extending contiguously from a nucleotide sequence corresponding to the 5′ end of SEQ ID NO:173 to a nucleotide sequence corresponding to the 3′ end of SEQ ID NO:173, but excluding the poly(A) tail at the 3′ end of SEQ ID NO:173. Also preferably the polynucleotide isolated according to the above process comprises a nucleotide sequence corresponding to the cDNA sequence of SEQ ID NO:173 from nucleotide 105 to nucleotide 1007, and extending contiguously from a nucleotide sequence corresponding to the 5′ end of said sequence of SEQ ID NO:173 from nucleotide 105 to nucleotide 1007, to a nucleotide sequence corresponding to the 3′ end of said sequence of SEQ ID NO:173 from nucleotide 105 to nucleotide 1007. Also preferably the polynucleotide isolated according to the above process comprises a nucleotide sequence corresponding to the cDNA sequence of SEQ ID NO:173 from nucleotide 801 to nucleotide 1007, and extending contiguously from a nucleotide sequence corresponding to the 5′ end of said sequence of SEQ ID NO:173 from nucleotide 801 to nucleotide 1007, to a nucleotide sequence corresponding to the 3′ end of said sequence of SEQ ID NO:173 from nucleotide 801 to nucleotide 1007. Also preferably the polynucleotide isolated according to the above process comprises a nucleotide sequence corresponding to the cDNA sequence of SEQ ID NO:173 from nucleotide 1 to nucleotide 352, and extending contiguously from a nucleotide sequence corresponding to the 5′ end of said sequence of SEQ ID NO:173 from nucleotide 1 to nucleotide 352, to a nucleotide sequence corresponding to the 3′ end of said sequence of SEQ ID NO:173 from nucleotide 1 to nucleotide 352.

In other embodiments, the present invention provides a composition comprising a protein, wherein said protein comprises an amino acid sequence selected from the group consisting of:
(a) the amino acid sequence of SEQ ID NO:174;
(b) the amino acid sequence of SEQ ID NO:174 from amino acid 1 to amino acid 83;
(c) a fragment of the amino acid sequence of SEQ ID NO:174, the fragment comprising eight contiguous amino acids of SEQ ID NO:174; and
(d) the amino acid sequence encoded by the cDNA insert of clone CW1155_{—}3 deposited with the ATCC under accession number 98279; the protein being substantially free from other mammalian proteins. Preferably such protein comprises the amino acid sequence of SEQ ID NO:174 or the amino acid sequence of SEQ ID NO:174 from amino acid 1 to amino acid 83. In further preferred embodiments, the present invention provides a protein comprising a fragment of the amino acid sequence of SEQ ID NO:174 having biological activity, the fragment preferably comprising eight (more preferably twenty, most preferably thirty) contiguous amino acids of SEQ ID NO:174, or a protein comprising a fragment of the amino acid sequence of SEQ ID NO:174 having biological activity, the fragment comprising the amino acid sequence from amino acid 145 to amino acid 154 of SEQ ID NO:174.

In one embodiment, the present invention provides a composition comprising an isolated polynucleotide selected from the group consisting of:
(a) a polynucleotide comprising the nucleotide sequence of SEQ ID NO:175;
(b) a polynucleotide comprising the nucleotide sequence of SEQ ID NO:175 from nucleotide 11 to nucleotide 1699;
(c) a polynucleotide comprising the nucleotide sequence of SEQ ID NO:175 from nucleotide 1682 to nucleotide 1699;
(d) a polynucleotide comprising the nucleotide sequence of SEQ ID NO:175 from nucleotide 737 to nucleotide 1134;
(e) a polynucleotide comprising the nucleotide sequence of the full-length protein coding sequence of clone CZ247_{—}2 deposited with the ATCC under accession number 98279;
(f) a polynucleotide encoding the full-length protein encoded by the cDNA insert of clone CZ247_{—}2 deposited with the ATCC under accession number 98279;
(g) a polynucleotide comprising the nucleotide sequence of a mature protein coding sequence of clone CZ247_{—}2 deposited with the ATCC under accession number 98279;
(h) a polynucleotide encoding a mature protein encoded by the cDNA insert of clone CZ247_{—}2 deposited with the ATCC under accession number 98279;
(i) a polynucleotide encoding a protein comprising the amino acid sequence of SEQ ID NO:176;
(j) a polynucleotide encoding a protein comprising a fragment of the amino acid sequence of SEQ ID NO:176 having biological activity, the fragment comprising eight contiguous amino acids of SEQ ID NO:176;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:175.

Preferably, such polynucleotide comprises the nucleotide sequence of SEQ ID NO:175 from nucleotide 11 to nucleotide 1699; the nucleotide sequence of SEQ ID NO:175 from nucleotide 1682 to nucleotide 1699; the nucleotide sequence of SEQ ID NO:175 from nucleotide 737 to nucleotide 1134; the nucleotide sequence of the full-length protein coding sequence of clone CZ247_{—}2 deposited with the ATCC under accession number 98279; or the nucleotide sequence of a mature protein coding sequence of clone CZ247_{—}2 deposited with the ATCC under accession number 98279. In other preferred embodiments, the polynucleotide encodes the full-length or a mature protein encoded by the cDNA insert of clone CZ247_{—}2 deposited with the ATCC under accession number 98279. In yet other preferred embodiments, the present invention provides a polynucleotide encoding a protein comprising the amino acid sequence of SEQ ID NO:176 from amino acid 298 to amino acid 374. In further preferred embodiments, the present invention provides a polynucleotide encoding a protein comprising a fragment of the amino acid sequence of SEQ ID NO:176 having biological activity, the fragment preferably comprising eight (more preferably twenty, most preferably thirty) contiguous amino acids of SEQ ID NO:176, or a polynucleotide encoding a protein comprising a fragment of the amino acid sequence of SEQ ID NO:176 having biological activity, the fragment comprising the amino acid sequence from amino acid 276 to amino acid 285 of SEQ ID NO:176.

Other embodiments provide the gene corresponding to the cDNA sequence of SEQ ID NO:175.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:175, but excluding the poly(A) tail at the 3′ end of SEQ ID NO:175; and
      (ab) the nucleotide sequence of the cDNA insert of clone CZ2472 deposited with the ATCC under accession number 98279;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:175, but excluding the poly(A) tail at the 3′ end of SEQ ID NO:175; and
      (bb) the nucleotide sequence of the cDNA insert of clone CZ2472 deposited with the ATCC under accession number 98279;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).

Preferably the polynucleotide isolated according to the above process comprises a nucleotide sequence corresponding to the cDNA sequence of SEQ ID NO:175, and extending contiguously from a nucleotide sequence corresponding to the 5′ end of SEQ ID NO:175 to a nucleotide sequence corresponding to the 3′ end of SEQ ID NO:175, but excluding the poly(A) tail at the 3′ end of SEQ ID NO:175. Also preferably the polynucleotide isolated according to the above process comprises a nucleotide sequence corresponding to the cDNA sequence of SEQ ID NO:175 from nucleotide 11 to nucleotide 1699, and extending contiguously from a nucleotide sequence corresponding to the 5′ end of said sequence of SEQ ID NO:175 from nucleotide 11 to nucleotide 1699, to a nucleotide sequence corresponding to the 3′ end of said sequence of SEQ ID NO:175 from nucleotide 11 to nucleotide 1699. Also preferably the polynucleotide isolated according to the above process comprises a nucleotide sequence corresponding to the cDNA sequence of SEQ ID NO:175 from nucleotide 1682 to nucleotide 1699, and extending contiguously from a nucleotide sequence corresponding to the 5′ end of said sequence of SEQ ID NO:175 from nucleotide 1682 to nucleotide 1699, to a nucleotide sequence corresponding to the 3′ end of said sequence of SEQ ID NO:175 from nucleotide 1682 to nucleotide 1699. Also preferably the polynucleotide isolated according to the above process comprises a nucleotide sequence corresponding to the cDNA sequence of SEQ ID NO:175 from nucleotide 737 to nucleotide 1134, and extending contiguously from a nucleotide sequence corresponding to the 5′ end of said sequence of SEQ ID NO:175 from nucleotide 737 to nucleotide 1134, to a nucleotide sequence corresponding to the 3′ end of said sequence of SEQ ID NO:175 from nucleotide 737 to nucleotide 1134.

In other embodiments, the present invention provides a composition comprising a protein, wherein said protein comprises an amino acid sequence selected from the group consisting of:
(a) the amino acid sequence of SEQ ID NO:176;
(b) the amino acid sequence of SEQ ID NO:176 from amino acid 298 to amino acid 374;
(c) a fragment of the amino acid sequence of SEQ ID NO:176, the fragment comprising eight contiguous amino acids of SEQ ID NO:176; and
(d) the amino acid sequence encoded by the cDNA insert of clone CZ247_{—}2 deposited with the ATCC under accession number 98279;
   the protein being substantially free from other mammalian proteins. Preferably such protein comprises the amino acid sequence of SEQ ID NO:176 or the amino acid sequence of SEQ ID NO:176 from amino acid 298 to amino acid 374. In further preferred embodiments, the present invention provides a protein comprising a fragment of the amino acid sequence of SEQ ID NO:176 having biological activity, the fragment preferably comprising eight (more preferably twenty, most preferably thirty) contiguous amino acids of SEQ ID NO:176, or a protein comprising a fragment of the amino add sequence of SEQ ID NO:176 having biological activity, the fragment comprising the amino acid sequence from amino acid 276 to amino acid 285 of SEQ ID NO:176.

In one embodiment, the present invention provides a composition comprising an isolated polynucleotide selected from the group consisting of:
(a) a polynucleotide comprising the nucleotide sequence of SEQ ID NO:177;
(b) a polynucleotide comprising the nucleotide sequence of SEQ ID NO:177 from nucleotide 918 to nucleotide 1262;
(c) a polynucleotide comprising the nucleotide sequence of SEQ ID NO:177 from nucleotide 999 to nucleotide 1262;
(d) a polynucleotide comprising the nucleotide sequence of SEQ ID NO:177 from nucleotide 928 to nucleotide 1134;
(e) a polynucleotide comprising the nucleotide sequence of the full-length protein coding sequence of clone AM666_{—}1 deposited with the ATCC under accession number 98292;
(f) a polynucleotide encoding the full-length protein encoded by the cDNA insert of clone AM666_{—}1 deposited with the ATCC under accession number 98292;
(g) a polynucleotide comprising the nucleotide sequence of a mature protein coding sequence of clone AM666_{—}1 deposited with the ATCC under accession number 98292;
(h) a polynucleotide encoding a mature protein encoded by the cDNA insert of clone AM666_{—}1 deposited with the ATCC under accession number 98292;
(i) a polynucleotide encoding a protein comprising the amino acid sequence of SEQ ID NO:178;
(j) a polynucleotide encoding a protein comprising a fragment of the amino acid sequence of SEQ ID NO:178 having biological activity, the fragment comprising eight contiguous amino acids of SEQ ID NO:178;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:177.

Preferably, such polynucleotide comprises the nucleotide sequence of SEQ ID NO:177 from nucleotide 918 to nucleotide 1262; the nucleotide sequence of SEQ ID NO:177 from nucleotide 999 to nucleotide 1262; the nucleotide sequence of SEQ ID NO:177 from nucleotide 928 to nucleotide 1134; the nucleotide sequence of the full-length protein coding sequence of clone AM666_{—}1 deposited with the ATCC under accession number 98292; or the nucleotide sequence of a mature protein coding sequence of clone AM666_{—}1 deposited with the ATCC under accession number 98292. In other preferred embodiments, the polynucleotide encodes the full-length or a mature protein encoded by the cDNA insert of clone AM666_{—}1 deposited with the ATCC under accession number 98292. In yet other preferred embodiments, the present invention provides a polynucleotide encoding a protein comprising the amino acid sequence of SEQ ID NO:178 from amino acid 5 to amino acid 72. In further preferred embodiments, the present invention provides a polynucleotide encoding a protein comprising a fragment of the amino acid sequence of SEQ ID NO:178 having biological activity, the fragment preferably comprising eight (more preferably twenty, most preferably thirty) contiguous amino acids of SEQ ID NO:178, or a polynucleotide encoding a protein comprising a fragment of the amino acid sequence of SEQ ID NO:178 having biological activity, the fragment comprising the amino acid sequence from amino add 52 to amino acid 61 of SEQ ID NO:178.

Other embodiments provide the gene corresponding to the cDNA sequence of SEQ ID NO:177.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:177, but excluding the poly(A) tail at the 3′ end of SEQ ID NO:177; and
      (ab) the nucleotide sequence of the cDNA insert of clone AM666_{—}1 deposited with the ATCC under accession number 98292;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:177, but excluding the poly(A) tail at the 3′ end of SEQ ID NO:177; and
      (bb) the nucleotide sequence of the cDNA insert of clone AM666_{—}1 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:177, and extending contiguously from a nucleotide sequence corresponding to the 5′ end of SEQ ID NO:177 to a nucleotide sequence corresponding to the 3′ end of SEQ ID NO:177, but excluding the poly(A) tail at the 3′ end of SEQ ID NO:177. Also preferably the polynucleotide isolated according to the above process comprises a nucleotide sequence corresponding to the cDNA sequence of SEQ ID NO:177 from nucleotide 918 to nucleotide 1262, and extending contiguously from a nucleotide sequence corresponding to the 5′ end of said sequence of SEQ ID NO:177 from nucleotide 918 to nucleotide 1262, to a nucleotide sequence corresponding to the 3′ end of said sequence of SEQ ID NO:177 from nucleotide 918 to nucleotide 1262. Also preferably the polynucleotide isolated according to the above process comprises a nucleotide sequence corresponding to the cDNA sequence of SEQ ID NO:177 from nucleotide 999 to nucleotide 1262, and extending contiguously from a nucleotide sequence corresponding to the 5′ end of said sequence of SEQ ID NO:177 from nucleotide 999 to nucleotide 1262, to a nucleotide sequence corresponding to the 3′ end of said sequence of SEQ ID NO:177 from nucleotide 999 to nucleotide 1262. Also preferably the polynucleotide isolated according to the above process comprises a nucleotide sequence corresponding to the cDNA sequence of SEQ ID NO:177 from nucleotide 928 to nucleotide 1134, and extending contiguously from a nucleotide sequence corresponding to the 5′ end of said sequence of SEQ ID NO:177 from nucleotide 928 to nucleotide 1134, to a nucleotide sequence corresponding to the 3′ end of said sequence of SEQ ID NO:177 from nucleotide 928 to nucleotide 1134.

In other embodiments, the present invention provides a composition comprising a protein, wherein said protein comprises an amino acid sequence selected from the group consisting of:
(a) the amino acid sequence of SEQ ID NO:178;
(b) the amino add sequence of SEQ ID NO:178 from amino acid 5 to amino acid 72;
(c) a fragment of the amino acid sequence of SEQ ID NO:178, the fragment comprising eight contiguous amino acids of SEQ ID NO:178; and
(d) the amino acid sequence encoded by the cDNA insert of clone AM666_{—}1 deposited with the ATCC under accession number 98292;
   the protein being substantially free from other mammalian proteins. Preferably such protein comprises the amino acid sequence of SEQ ID NO:178 or the amino acid sequence of SEQ ID NO:178 from amino acid 5 to amino acid 72. In further preferred embodiments, the present invention provides a protein comprising a fragment of the amino acid sequence of SEQ ID NO:178 having biological activity, the fragment preferably comprising eight (more preferably twenty, most preferably thirty) contiguous amino acids of SEQ ID NO:178, or a protein comprising a fragment of the amino acid sequence of SEQ ID NO:178 having biological activity, the fragment comprising the amino acid sequence from amino acid 52 to amino acid 61 of SEQ ID NO:178.

In one embodiment, the present invention provides a composition comprising an isolated polynucleotide selected from the group consisting of:
(a) a polynucleotide comprising the nucleotide sequence of SEQ ID NO:179;
(b) a polynucleotide comprising the nucleotide sequence of SEQ ID NO:179 from nucleotide 751 to nucleotide 906;
(c) a polynucleotide comprising the nucleotide sequence of SEQ ID NO:179 from nucleotide 829 to nucleotide 906;
(d) a polynucleotide comprising the nucleotide sequence of SEQ ID NO:179 from nucleotide 556 to nucleotide 831;
(e) a polynucleotide comprising the nucleotide sequence of the full-length protein coding sequence of clone BN387_{—}3 deposited with the ATCC under accession number 98292;
(f) a polynucleotide encoding the full-length protein encoded by the cDNA insert of clone BN387_{—}3 deposited with the ATCC under accession number 98292;
(g) a polynucleotide comprising the nucleotide sequence of a mature protein coding sequence of clone BN387_{—}3 deposited with the ATCC under accession number 98292;
(h) a polynucleotide encoding a mature protein encoded by the cDNA insert of clone BN387_{—}3 deposited with the ATCC under accession number 98292;
(i) a polynucleotide encoding a protein comprising the amino acid sequence of SEQ ID NO:180;
(j) a polynucleotide encoding a protein comprising a fragment of the amino acid sequence of SEQ ID NO:180 having biological activity, the fragment comprising eight contiguous amino acids of SEQ ID NO:180;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:179.

Preferably, such polynucleotide comprises the nucleotide sequence of SEQ ID NO:179 from nucleotide 751 to nucleotide 906; the nucleotide sequence of SEQ ID NO:179 from nucleotide 829 to nucleotide 906; the nucleotide sequence of SEQ ID NO:179 from nucleotide 556 to nucleotide 831; the nucleotide sequence of the full-length protein coding sequence of clone BN387_{—}3 deposited with the ATCC under accession number 98292; or the nucleotide sequence of a mature protein coding sequence of clone BN387_{—}3 deposited with the ATCC under accession number 98292. In other preferred embodiments, the polynucleotide encodes the full-length or a mature protein encoded by the cDNA insert of clone BN387_{—}3 deposited with the ATCC under accession number 98292. In yet other preferred embodiments, the present invention provides a polynucleotide encoding a protein comprising the amino acid sequence of SEQ ID NO:180 from amino acid 1 to amino acid 27. In further preferred embodiments, the present invention provides a polynucleotide encoding a protein comprising a fragment of the amino acid sequence of SEQ ID NO:180 having biological activity, the fragment preferably comprising eight (more preferably twenty, most preferably thirty) contiguous amino acids of SEQ ID NO:180, or a polynucleotide encoding a protein comprising a fragment of the amino acid sequence of SEQ ID NO:180 having biological activity, the fragment comprising the amino acid sequence from amino acid 21 to amino acid 30 of SEQ ID NO:180.

Other embodiments provide the gene corresponding to the cDNA sequence of SEQ ID NO:179.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:179, but excluding the poly(A) tail at the 3′ end of SEQ ID NO:179; and
      (ab) the nucleotide sequence of the cDNA insert of clone BN387_{—}3 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:179, but excluding the poly(A) tail at the 3′ end of SEQ ID NO:179; and
      (bb) the nucleotide sequence of the cDNA insert of clone BN387_{—}3 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:179, and extending contiguously from a nucleotide sequence corresponding to the 5′ end of SEQ ID NO:179 to a nucleotide sequence corresponding to the 3′ end of SEQ ID NO:179, but excluding the poly(A) tail at the 3′ end of SEQ ID NO:179. Also preferably the polynucleotide isolated according to the above process comprises a nucleotide sequence corresponding to the cDNA sequence of SEQ ID NO:179 from nucleotide 751 to nucleotide 906, and extending contiguously from a nucleotide sequence corresponding to the 5′ end of said sequence of SEQ ID NO:179 from nucleotide 751 to nucleotide 906, to a nucleotide sequence corresponding to the 3′ end of said sequence of SEQ ID NO:179 from nucleotide 751 to nucleotide 906. Also preferably the polynucleotide isolated according to the above process comprises a nucleotide sequence corresponding to the cDNA sequence of SEQ ID NO:179 from nucleotide 829 to nucleotide 906, and extending contiguously from a nucleotide sequence corresponding to the 5′ end of said sequence of SEQ ID NO:179 from nucleotide 829 to nucleotide 906, to a nucleotide sequence corresponding to the 3′ end of said sequence of SEQ ID NO:179 from nucleotide 829 to nucleotide 906. Also preferably the polynucleotide isolated according to the above process comprises a nucleotide sequence corresponding to the cDNA sequence of SEQ ID NO:179 from nucleotide 556 to nucleotide 831, and extending contiguously from a nucleotide sequence corresponding to the 5′ end of said sequence of SEQ ID NO:179 from nucleotide 556 to nucleotide 831, to a nucleotide sequence corresponding to the 3′ end of said sequence of SEQ ID NO:179 from nucleotide 556 to nucleotide 831.

In other embodiments, the present invention provides a composition comprising a protein, wherein said protein comprises an amino acid sequence selected from the group consisting of:
(a) the amino acid sequence of SEQ ID NO:180;
(b) the amino acid sequence of SEQ ID NO:180 from amino acid 1 to amino acid 27;
(c) a fragment of the amino add sequence of SEQ ID NO:180, the fragment comprising eight contiguous amino acids of SEQ ID NO:180; and
(d) the amino acid sequence encoded by the cDNA insert of clone BN387_{—}3 deposited with the ATCC under accession number 98292;
   the protein being substantially free from other mammalian proteins. Preferably such protein comprises the amino acid sequence of SEQ ID NO:180 or the amino add sequence of SEQ ID NO:180 from amino acid 1 to amino acid 27. In further preferred embodiments, the present invention provides a protein comprising a fragment of the amino acid sequence of SEQ ID NO:180 having biological activity, the fragment preferably comprising eight (more preferably twenty, most preferably thirty) contiguous amino acids of SEQ ID NO:180, or a protein comprising a fragment of the amino add sequence of SEQ ID NO:180 having biological activity, the fragment comprising the amino acid sequence from amino acid 21 to amino acid 30 of SEQ ID NO:180.

In one embodiment, the present invention provides a composition comprising an isolated polynucleotide selected from the group consisting of:
(a) a polynucleotide comprising the nucleotide sequence of SEQ ID NO:181;
(b) a polynucleotide comprising the nucleotide sequence of SEQ ID NO:181 from nucleotide 139 to nucleotide 765;
(c) a polynucleotide comprising the nucleotide sequence of SEQ ID NO:181 from nucleotide 1 to nucleotide 416;
(d) a polynucleotide comprising the nucleotide sequence of the full-length protein coding sequence of clone BQ135_{—}2 deposited with the ATCC under accession number 98292; (e) a polynucleotide encoding the full-length protein encoded by the cDNA insert of clone BQ135_{—}2 deposited with the ATCC under accession number 98292;
(f) a polynucleotide comprising the nucleotide sequence of a mature protein coding sequence of clone BQ135_{—}2 deposited with the ATCC under accession number 98292;
(g) a polynucleotide encoding a mature protein encoded by the cDNA insert of clone BQ135_{—}2 deposited with the ATCC under accession number 98292;
(h) a polynucleotide encoding a protein comprising the amino acid sequence of SEQ ID NO:182;
(i) a polynucleotide encoding a protein comprising a fragment of the amino acid sequence of SEQ ID NO:182 having biological activity, the fragment comprising eight contiguous amino acids of SEQ ID NO:182;
a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:181.

Preferably, such polynucleotide comprises the nucleotide sequence of SEQ ID NO:181 from nucleotide 139 to nucleotide 765; the nucleotide sequence of SEQ ID NO:181 from nucleotide 1 to nucleotide 416; the nucleotide sequence of the full-length protein coding sequence of clone BQ135_{—}2 deposited with the ATCC under accession number 98292; or the nucleotide sequence of a mature protein coding sequence of clone BQ135_{—}2 deposited with the ATCC under accession number 98292. In other preferred embodiments, the polynucleotide encodes the full-length or a mature protein encoded by the cDNA insert of clone BQ135_{—}2 deposited with the ATCC under accession number 98292. In yet other preferred embodiments, the present invention provides a polynucleotide encoding a protein comprising the amino acid sequence of SEQ ID NO:182 from amino acid 1 to amino acid 93. In further preferred embodiments, the present invention provides a polynucleotide encoding a protein comprising a fragment of the amino acid sequence of SEQ ID NO:182 having biological activity, the fragment preferably comprising eight (more preferably twenty, most preferably thirty) contiguous amino acids of SEQ ID NO:182, or a polynucleotide encoding a protein comprising a fragment of the amino acid sequence of SEQ ID NO:182 having biological activity, the fragment comprising the amino acid sequence from amino acid 99 to amino acid 108 of SEQ ID NO:182.

Other embodiments provide the gene corresponding to the cDNA sequence of SEQ ID NO:181.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:181, but excluding the poly(A) tail at the 3′ end of SEQ ID NO:181; and
      (ab) the nucleotide sequence of the cDNA insert of clone BQ135_{—}2 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:181, but excluding the poly(A) tail at the 3′ end of SEQ ID NO:181; and
      (bb) the nucleotide sequence of the cDNA insert of clone BQ135_{—}2 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:181, and extending contiguously from a nucleotide sequence corresponding to the 5′ end of SEQ ID NO:181 to a nucleotide sequence corresponding to the 3′ end of SEQ ID NO:181, but excluding the poly(A) tail at the 3′ end of SEQ ID NO:181. Also preferably the polynucleotide isolated according to the above process comprises a nucleotide sequence corresponding to the cDNA sequence of SEQ ID NO:181 from nucleotide 139 to nucleotide 765, and extending contiguously from a nucleotide sequence corresponding to the 5′ end of said sequence of SEQ ID NO:181 from nucleotide 139 to nucleotide 765, to a nucleotide sequence corresponding to the 3′ end of said sequence of SEQ ID NO:181 from nucleotide 139 to nucleotide 765. Also preferably the polynucleotide isolated according to the above process comprises a nucleotide sequence corresponding to the cDNA sequence of SEQ ID NO:181 from nucleotide 1 to nucleotide 416, and extending contiguously from a nucleotide sequence corresponding to the 5′ end of said sequence of SEQ ID NO:181 from nucleotide 1 to nucleotide 416, to a nucleotide sequence corresponding to the 3′ end of said sequence of SEQ ID NO:181 from nucleotide 1 to nucleotide 416.

In other embodiments, the present invention provides a composition comprising a protein, wherein said protein comprises an amino acid sequence selected from the group consisting of:
(a) the amino acid sequence of SEQ ID NO:182;
(b) the amino acid sequence of SEQ ID NO:182 from amino acid 1 to amino acid 93;
(c) a fragment of the amino acid sequence of SEQ ID NO:182, the fragment comprising eight contiguous amino adds of SEQ ID NO:182; and
(d) the amino acid sequence encoded by the cDNA insert of clone BQ135_{—}2 deposited with the ATCC under accession number 98292;
   the protein being substantially free from other mammalian proteins. Preferably such protein comprises the amino acid sequence of SEQ ID NO:182 or the amino acid sequence of SEQ ID NO:182 from amino acid 1 to amino acid 93. In further preferred embodiments, the present invention provides a protein comprising a fragment of the amino acid sequence of SEQ ID NO:182 having biological activity, the fragment preferably comprising eight (more preferably twenty, most preferably thirty) contiguous amino acids of SEQ ID NO:182, or a protein comprising a fragment of the amino acid sequence of SEQ ID NO:182 having biological activity, the fragment comprising the amino acid sequence from amino acid 99 to amino acid 108 of SEQ ID NO:182.

In one embodiment, the present invention provides a composition comprising an isolated polynucleotide selected from the group consisting of:
(a) a polynucleotide comprising the nucleotide sequence of SEQ ID NO:183;
(b) a polynucleotide comprising the nucleotide sequence of SEQ ID NO:183 from nucleotide 214 to nucleotide 714;
(c) a polynucleotide comprising the nucleotide sequence of SEQ ID NO:183 from nucleotide 151 to nucleotide 531;
(d) a polynucleotide comprising the nucleotide sequence of the full-length protein coding sequence of clone CR678_{—}1 deposited with the ATCC under accession number 98292;
(e) a polynucleotide encoding the full-length protein encoded by the cDNA insert of clone CR678_{—}1 deposited with the ATCC under accession number 98292;
(f) a polynucleotide comprising the nucleotide sequence of a mature protein coding sequence of clone CR678_{—}1 deposited with the ATCC under accession number 98292;
(g) a polynucleotide encoding a mature protein encoded by the cDNA insert of clone CR678_{—}1 deposited with the ATCC under accession number 98292;
(h) a polynucleotide encoding a protein comprising the amino acid sequence of SEQ ID NO:184;
(i) a polynucleotide encoding a protein comprising a fragment of the amino acid sequence of SEQ ID NO:184 having biological activity, the fragment comprising eight contiguous amino acids of SEQ ID NO:184;
a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:183.

Preferably, such polynucleotide comprises the nucleotide sequence of SEQ ID NO:183 from nucleotide 214 to nucleotide 714; the nucleotide sequence of SEQ ID NO:183 from nucleotide 151 to nucleotide 531; the nucleotide sequence of the full-length protein coding sequence of clone CR678_{—}1 deposited with the ATCC under accession number 98292; or the nucleotide sequence of a mature protein coding sequence of clone CR678_{—}1 deposited with the ATCC under accession number 98292. In other preferred embodiments, the polynucleotide encodes the full-length or a mature protein encoded by the cDNA insert of clone CR678_{—}1 deposited with the ATCC under accession number 98292. In yet other preferred embodiments, the present invention provides a polynucleotide encoding a protein comprising the amino acid sequence of SEQ ID NO:184 from amino acid 1 to amino acid 106. In further preferred embodiments, the present invention provides a polynucleotide encoding a protein comprising a fragment of the amino acid sequence of SEQ ID NO:184 having biological activity, the fragment preferably comprising eight (more preferably twenty, most preferably thirty) contiguous amino acids of SEQ ID NO:184, or a polynucleotide encoding a protein comprising a fragment of the amino acid sequence of SEQ ID NO:184 having biological activity, the fragment comprising the amino acid sequence from amino acid 78 to amino acid 87 of SEQ ID NO:184.

Other embodiments provide the gene corresponding to the cDNA sequence of SEQ ID NO:183.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:183, but excluding the poly(A) tail at the 3′ end of SEQ ID NO:183; and
      (ab) the nucleotide sequence of the cDNA insert of clone CR678_{—}1 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:183, but excluding the poly(A) tail at the 3′ end of SEQ ID NO:183; and
      (bb) the nucleotide sequence of the cDNA insert of clone CR678_{—}1 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:183, and extending contiguously from a nucleotide sequence corresponding to the 5′ end of SEQ ID NO:183 to a nucleotide sequence corresponding to the 3′ end of SEQ ID NO:183, but excluding the poly(A) tail at the 3′ end of SEQ ID NO:183. Also preferably the polynucleotide isolated according to the above process comprises a nucleotide sequence corresponding to the cDNA sequence of SEQ ID NO:183 from nucleotide 214 to nucleotide 714, and extending contiguously from a nucleotide sequence corresponding to the 5′ end of said sequence of SEQ ID NO:183 from nucleotide 214 to nucleotide 714, to a nucleotide sequence corresponding to the 3′ end of said sequence of SEQ ID NO:183 from nucleotide 214 to nucleotide 714. Also preferably the polynucleotide isolated according to the above process comprises a nucleotide sequence corresponding to the cDNA sequence of SEQ ID NO:183 from nucleotide 151 to nucleotide 531, and extending contiguously from a nucleotide sequence corresponding to the 5′ end of said sequence of SEQ ID NO:183 from nucleotide 151 to nucleotide 531, to a nucleotide sequence corresponding to the 3′ end of said sequence of SEQ ID NO:183 from nucleotide 151 to nucleotide 531.

In other embodiments, the present invention provides a composition comprising a protein, wherein said protein comprises an amino acid sequence selected from the group consisting of:
(a) the amino acid sequence of SEQ ID NO:184;
(b) the amino acid sequence of SEQ ID NO:184 from amino acid 1 to amino acid 106;
(c) a fragment of the amino acid sequence of SEQ ID NO:184, the fragment comprising eight contiguous amino acids of SEQ ID NO:184; and
(d) the amino acid sequence encoded by the cDNA insert of clone CR678_{—}1 deposited with the ATCC under accession number 98292;
   the protein being substantially free from other mammalian proteins. Preferably such protein comprises the amino acid sequence of SEQ ID NO:184 or the amino acid sequence of SEQ ID NO:184 from amino acid 1 to amino acid 106. In further preferred embodiments, the present invention provides a protein comprising a fragment of the amino acid sequence of SEQ ID NO:184 having biological activity, the fragment preferably comprising eight (more preferably twenty, most preferably thirty) contiguous amino acids of SEQ ID NO:184, or a protein comprising a fragment of the amino acid sequence of SEQ ID NO:184 having biological activity, the fragment comprising the amino acid sequence from amino acid 78 to amino acid 87 of SEQ ID NO:184.

In one embodiment, the present invention provides a composition comprising an isolated polynucleotide selected from the group consisting of:
(a) a polynucleotide comprising the nucleotide sequence of SEQ ID NO:185;
(b) a polynucleotide comprising the nucleotide sequence of SEQ ID NO:185 from nucleotide 116 to nucleotide 4498;
(c) a polynucleotide comprising the nucleotide sequence of SEQ ID NO:185 from nucleotide 1221 to nucleotide 1711;
(d) a polynucleotide comprising the nucleotide sequence of the full-length protein coding sequence of clone CW420_{—}2 deposited with the ATCC under accession number 98292;
(e) a polynucleotide encoding the full-length protein encoded by the cDNA insert of clone CW420_{—}2 deposited with the ATCC under accession number 98292;
(f) a polynucleotide comprising the nucleotide sequence of a mature protein coding sequence of clone CW420_{—}2 deposited with the ATCC under accession number 98292;
(g) a polynucleotide encoding a mature protein encoded by the cDNA insert of clone CW420_{—}2 deposited with the ATCC under accession number 98292;
(h) a polynucleotide encoding a protein comprising the amino acid sequence of SEQ ID NO:186;
(i) a polynucleotide encoding a protein comprising a fragment of the amino acid sequence of SEQ ID NO:186 having biological activity, the fragment comprising eight contiguous amino acids of SEQ ID NO:186;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:185.

Preferably, such polynucleotide comprises the nucleotide sequence of SEQ ID NO:185 from nucleotide 116 to nucleotide 4498; the nucleotide sequence of SEQ ID NO:185 from nucleotide 1221 to nucleotide 1711; the nucleotide sequence of the full-length protein coding sequence of clone CW420_{—}2 deposited with the ATCC under accession number 98292; or the nucleotide sequence of a mature protein coding sequence of clone CW420_{—}2 deposited with the ATCC under accession number 98292. In other preferred embodiments, the polynucleotide encodes the full-length or a mature protein encoded by the cDNA insert of clone CW420_{—}2 deposited with the ATCC under accession number 98292. In yet other preferred embodiments, the present invention provides a polynucleotide encoding a protein comprising the amino acid sequence of SEQ ID NO:186 from amino acid 370 to amino acid 532. In further preferred embodiments, the present invention provides a polynucleotide encoding a protein comprising a fragment of the amino acid sequence of SEQ ID NO:186 having biological activity, the fragment preferably comprising eight (more preferably twenty, most preferably thirty) contiguous amino acids of SEQ ID NO:186, or a polynucleotide encoding a protein comprising a fragment of the amino acid sequence of SEQ ID NO:186 having biological activity, the fragment comprising the amino acid sequence from amino acid 725 to amino acid 734 of SEQ ID NO:186.

Other embodiments provide the gene corresponding to the cDNA sequence of SEQ ID NO:185.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:185, but excluding the poly(A) tail at the 3′ end of SEQ ID NO:185; and
      (ab) the nucleotide sequence of the cDNA insert of clone CW420_{—}2 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:185, but excluding the poly(A) tail at the 3′ end of SEQ ID NO:185; and
      (bb) the nucleotide sequence of the cDNA insert of clone CW420_{—}2 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:185, and extending contiguously from a nucleotide sequence corresponding to the 5′ end of SEQ ID NO:185 to a nucleotide sequence corresponding to the 3′ end of SEQ ID NO:185, but excluding the poly(A) tail at the 3′ end of SEQ ID NO:185. Also preferably the polynucleotide isolated according to the above process comprises a nucleotide sequence corresponding to the cDNA sequence of SEQ ID NO:185 from nucleotide 116 to nucleotide 4498, and extending contiguously from a nucleotide sequence corresponding to the 5′ end of said sequence of SEQ ID NO:185 from nucleotide 116 to nucleotide 4498, to a nucleotide sequence corresponding to the 3′ end of said sequence of SEQ ID NO:185 from nucleotide 116 to nucleotide 4498. Also preferably the polynucleotide isolated according to the above process comprises a nucleotide sequence corresponding to the cDNA sequence of SEQ ID NO:185 from nucleotide 1221 to nucleotide 1711, and extending contiguously from a nucleotide sequence corresponding to the 5′ end of said sequence of SEQ ID NO:185 from nucleotide 1221 to nucleotide 1711, to a nucleotide sequence corresponding to the 3′ end of said sequence of SEQ ID NO:185 from nucleotide 1221 to nucleotide 1711.

In other embodiments, the present invention provides a composition comprising a protein, wherein said protein comprises an amino acid sequence selected from the group consisting of:
(a) the amino acid sequence of SEQ ID NO:186;
(b) the amino acid sequence of SEQ ID NO:186 from amino acid 370 to amino acid 532;
(c) a fragment of the amino acid sequence of SEQ ID NO:186, the fragment comprising eight contiguous amino acids of SEQ ID NO:186; and
(d) the amino acid sequence encoded by the cDNA insert of clone CW420_{—}2 deposited with the ATCC under accession number 98292;
   the protein being substantially free from other mammalian proteins. Preferably such protein comprises the amino acid sequence of SEQ ID NO:186 or the amino acid sequence of SEQ ID NO:186 from amino acid 370 to amino acid 532. In further preferred embodiments, the present invention provides a protein comprising a fragment of the amino acid sequence of SEQ ID NO:186 having biological activity, the fragment preferably comprising eight (more preferably twenty, most preferably thirty) contiguous amino adds of SEQ ID NO:186, or a protein comprising a fragment of the amino acid sequence of SEQ ID NO:186 having biological activity, the fragment comprising the amino acid sequence from amino acid 725 to amino acid 734 of SEQ ID NO:186.

In one embodiment, the present invention provides a composition comprising an isolated polynucleotide selected from the group consisting of:
(a) a polynucleotide comprising the nucleotide sequence of SEQ ID NO:187;
(b) a polynucleotide comprising the nucleotide sequence of SEQ ID NO:187 from nucleotide 119 to nucleotide 2176;
(c) a polynucleotide comprising the nucleotide sequence of SEQ ID NO:187 from nucleotide 1 to nucleotide 529;
(d) a polynucleotide comprising the nucleotide sequence of the full-length protein coding sequence of clone CW795_{—}2 deposited with the ATCC under accession number 98292;
(e) a polynucleotide encoding the full-length protein encoded by the cDNA insert of clone CW795_{—}2 deposited with the ATCC under accession number 98292;
(f) a polynucleotide comprising the nucleotide sequence of a mature protein coding sequence of clone CW795_{—}2 deposited with the ATCC under accession number 98292;
(g) a polynucleotide encoding a mature protein encoded by the cDNA insert of clone CW795_{—}2 deposited with the ATCC under accession number 98292;
(h) a polynucleotide encoding a protein comprising the amino acid sequence of SEQ ID NO:188;
(i) a polynucleotide encoding a protein comprising a fragment of the amino acid sequence of SEQ ID NO:188 having biological activity, the fragment comprising eight contiguous amino acids of SEQ ID NO:188;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:187.

Preferably, such polynucleotide comprises the nucleotide sequence of SEQ ID NO:187 from nucleotide 119 to nucleotide 2176; the nucleotide sequence of SEQ ID NO:187 from nucleotide 1 to nucleotide 529; the nucleotide sequence of the full-length protein coding sequence of clone CW795_{—}2 deposited with the ATCC under accession number 98292; or the nucleotide sequence of a mature protein coding sequence of clone CW795_{—}2 deposited with the ATCC under accession number 98292. In other preferred embodiments, the polynucleotide encodes the full-length or a mature protein encoded by the cDNA insert of clone CW795_{—}2 deposited with the ATCC under accession number 98292. In yet other preferred embodiments, the present invention provides a polynucleotide encoding a protein comprising the amino acid sequence of SEQ ID NO:188 from amino acid 1 to amino acid 137. In further preferred embodiments, the present invention provides a polynucleotide encoding a protein comprising a fragment of the amino acid sequence of SEQ ID NO:188 having biological activity, the fragment preferably comprising eight (more preferably twenty, most preferably thirty) contiguous amino acids of SEQ ID NO:188, or a polynucleotide encoding a protein comprising a fragment of the amino acid sequence of SEQ ID NO:188 having biological activity, the fragment comprising the amino acid sequence from amino add 338 to amino acid 347 of SEQ ID NO:188.

Other embodiments provide the gene corresponding to the cDNA sequence of SEQ ID NO:187.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:187, but excluding the poly(A) tail at the 3′ end of SEQ ID NO:187; and
      (ab) the nucleotide sequence of the cDNA insert of clone CW795_{—}2 deposited with the ATCC under accession number 98292;
   (ii) hybridizing said probe(s) to human genomic DNA in conditions at least as stringent as 4×SSC at 50 degrees C.; and
   (ii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:187, but excluding the poly(A) tail at the 3′ end of SEQ ID NO:187; and
      (bb) the nucleotide sequence of the cDNA inert of clone CW795_{—}2 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:187, and extending contiguously from a nucleotide sequence corresponding to the 5′ end of SEQ ID NO:187 to a nucleotide sequence corresponding to the 3′ end of SEQ ID NO:187, but excluding the poly(A) tail at the 3′ end of SEQ ID NO:187. Also preferably the polynucleotide isolated according to the above process comprises a nucleotide sequence corresponding to the cDNA sequence of SEQ ID NO:187 from nucleotide 119 to nucleotide 2176, and extending contiguously from a nucleotide sequence corresponding to the 5′ end of said sequence of SEQ ID NO:187 from nucleotide 119 to nucleotide 2176, to a nucleotide sequence corresponding to the 3′ end of said sequence of SEQ ID NO:187 from nucleotide 119 to nucleotide 2176. Also preferably the polynucleotide isolated according to the above process comprises a nucleotide sequence corresponding to the cDNA sequence of SEQ ID NO:187 from nucleotide 1 to nucleotide 529, and extending contiguously from a nucleotide sequence corresponding to the 5′ end of said sequence of SEQ ID NO:187 from nucleotide 1 to nucleotide 529, to a nucleotide sequence corresponding to the 3′ end of said sequence of SEQ ID NO:187 from nucleotide 1 to nucleotide 529.

In other embodiments, the present invention provides a composition comprising a protein, wherein said protein comprises an amino acid sequence selected from the group consisting of:
(a) the amino acid sequence of SEQ ID NO:188;
(b) the amino acid sequence of SEQ ID NO:188 from amino acid 1 to amino acid 137;
(c) a fragment of the amino acid sequence of SEQ ID NO:188, the fragment comprising eight contiguous amino acids of SEQ ID NO:188; and
(d) the amino acid sequence encoded by the cDNA insert of clone CW795_{—}2 deposited with the ATCC under accession number 98292;
   the protein being substantially free from other mammalian proteins. Preferably such protein comprises the amino acid sequence of SEQ ID NO:188 or the amino acid sequence of SEQ ID NO:188 from amino acid 1 to amino acid 137. In further preferred embodiments, the present invention provides a protein comprising a fragment of the amino acid sequence of SEQ ID NO:188 having biological activity, the fragment preferably comprising eight (more preferably twenty, most preferably thirty) contiguous amino acids of SEQ. ID NO:188, or a protein comprising a fragment of the amino acid sequence of SEQ ID NO:188 having biological activity, the fragment comprising the amino acid sequence from amino acid 338 to amino acid 347 of SEQ ID NO:188.

In one embodiment, the present invention provides a composition comprising an isolated polynucleotide selected from the group consisting of
(a) a polynucleotide comprising the nucleotide sequence of SEQ ID NO:189;
(b) a polynucleotide comprising the nucleotide sequence of SEQ ID NO:189 from nucleotide 401 to nucleotide 589;
(c) a polynucleotide comprising the nucleotide sequence of SEQ ID NO:189 from nucleotide 258 to nucleotide 627;
(d) a polynucleotide comprising the nucleotide sequence of the full-length protein coding sequence of clone CW823_{—}3 deposited with the ATCC under accession number 98292;
(e) a polynucleotide encoding the full-length protein encoded by the cDNA insert of clone CW823_{—}3 deposited with the ATCC under accession number 98292;
(f) a polynucleotide comprising the nucleotide sequence of a mature protein coding sequence of clone CW823_{—}3 deposited with the ATCC under accession number 98292;
(g) a polynucleotide encoding a mature protein encoded by the cDNA insert of clone CW823_{—}3 deposited with the ATCC under accession number 98292;
(h) a polynucleotide encoding a protein comprising the amino acid sequence of SEQ ID NO:190;
(i) a polynucleotide encoding a protein comprising a fragment of the amino acid sequence of SEQ ID NO:190 having biological activity, the fragment comprising eight contiguous amino adds of SEQ ID NO:190;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:189.

Preferably, such polynucleotide comprises the nucleotide sequence of SEQ ID NO:189 from nucleotide 401 to nucleotide 589; the nucleotide sequence of SEQ ID NO:189 from nucleotide 258 to nucleotide 627; the nucleotide sequence of the full-length protein coding sequence of clone CW823_{—}3 deposited with the ATCC under accession number 98292; or the nucleotide sequence of a mature protein coding sequence of clone CW823_{—}3 deposited with the ATCC under accession number 98292. In other preferred embodiments, the polynucleotide encodes the full-length or a mature protein encoded by the cDNA insert of clone CW823_{—}3 deposited with the ATCC under accession number 98292. In further preferred embodiments, the present invention provides a polynucleotide encoding a protein comprising a fragment of the amino acid sequence of SEQ ID NO:190 having biological activity, the fragment preferably comprising eight (more preferably twenty, most preferably thirty) contiguous amino acids of SEQ ID NO:190, or a polynucleotide encoding a protein comprising a fragment of the amino acid sequence of SEQ ID NO:190 having biological activity, the fragment comprising the amino acid sequence from amino acid 26 to amino acid 35 of SEQ ID NO:190.

Other embodiments provide the gene corresponding to the cDNA sequence of SEQ ID NO:189.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:189, but excluding the poly(A) tail at the 3′ end of SEQ ID NO:189; and
      (ab) the nucleotide sequence of the cDNA insert of clone CW823_{—}3 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:189, but excluding the poly(A) tail at the 3′ end of SEQ ID NO:189; and
      (bb) the nucleotide sequence of the cDNA insert of clone CW823_{—}3 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:189, and extending contiguously from a nucleotide sequence corresponding to the 5′ end of SEQ ID NO:189 to a nucleotide sequence corresponding to the 3′ end of SEQ ID NO:189, but excluding the poly(A) tail at the 3′ end of SEQ ID NO:189. Also preferably the polynucleotide isolated according to the above process comprises a nucleotide sequence corresponding to the cDNA sequence of SEQ ID NO:189 from nucleotide 401 to nucleotide 589, and extending contiguously from a nucleotide sequence corresponding to the 5′ end of said sequence of SEQ ID NO:189 from nucleotide 401 to nucleotide 589, to a nucleotide sequence corresponding to the 3′ end of said sequence of SEQ ID NO:189 from nucleotide 401 to nucleotide 589. Also preferably the polynucleotide isolated according to the above process comprises a nucleotide sequence corresponding to the cDNA sequence of SEQ ID NO:189 from nucleotide 258 to nucleotide 627, and extending contiguously from a nucleotide sequence corresponding to the 5′ end of said sequence of SEQ ID NO:189 from nucleotide 258 to nucleotide 627, to a nucleotide sequence corresponding to the 3′ end of said sequence of SEQ ID NO:189 from nucleotide 258 to nucleotide 627.

In other embodiments, the present invention provides a composition comprising a protein, wherein said protein comprises an amino acid sequence selected from the group consisting of:
(a) the amino acid sequence of SEQ ID NO:190;
(b) a fragment of the amino acid sequence of SEQ ID NO:190, the fragment comprising eight contiguous amino acids of SEQ ID NO:190; and
(c) the amino acid sequence encoded by the cDNA insert of clone CW823_{—}3 deposited with the ATCC under accession number 98292;
   the protein being substantially free from other mammalian proteins. Preferably such protein comprises the amino acid sequence of SEQ ID NO:190. In further preferred embodiments, the present invention provides a protein comprising a fragment of the amino acid sequence of SEQ ID NO:190 having biological activity, the fragment preferably comprising eight (more preferably twenty, most preferably thirty) contiguous amino acids of SEQ ID NO:190, or a protein comprising a fragment of the amino acid sequence of SEQ ID NO:190 having biological activity, the fragment comprising the amino acid sequence from amino acid 26 to amino acid 35 of SEQ ID NO:190.

In one embodiment, the present invention provides a composition comprising an isolated polynucleotide selected from the group consisting of:
(a) a polynucleotide comprising the nucleotide sequence of SEQ ID NO:191;
(b) a polynucleotide comprising the nucleotide sequence of SEQ ID NO:191 from nucleotide 548 to nucleotide 868;
(c) a polynucleotide comprising the nucleotide sequence of SEQ ID NO:191 from nucleotide 590 to nucleotide 868;
(d) a polynucleotide comprising the nucleotide sequence of the full-length protein coding sequence of clone DF989_{—}3 deposited with the ATCC under accession number 98292;
(e) a polynucleotide encoding the full-length protein encoded by the cDNA insert of clone DF989_{—}3 deposited with the ATCC under accession number 98292;
(f) a polynucleotide comprising the nucleotide sequence of a mature protein coding sequence of clone DF989_{—}3 deposited with the ATCC under accession number 98292;
(g) a polynucleotide encoding a mature protein encoded by the cDNA insert of clone DF989_{—}3 deposited with the ATCC under accession number 98292;
(h) a polynucleotide encoding a protein comprising the amino acid sequence of SEQ ID NO:192;
(i) a polynucleotide encoding a protein comprising a fragment of the amino acid sequence of SEQ ID NO:192 having biological activity, the fragment comprising eight contiguous amino acids of SEQ ID NO:192;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:191.

Preferably, such polynucleotide comprises the nucleotide sequence of SEQ ID NO:191 from nucleotide 548 to nucleotide 868; the nucleotide sequence of SEQ ID NO:191 from nucleotide 590 to nucleotide 868; the nucleotide sequence of the full-length protein coding sequence of clone DF989_{—}3 deposited with the ATCC under accession number 98292; or the nucleotide sequence of a mature protein coding sequence of clone DF989_{—}3 deposited with the ATCC under accession number 98292. In other preferred embodiments, the polynucleotide encodes the full-length or a mature protein encoded by the cDNA insert of clone DF989_{—}3 deposited with the ATCC under accession number 98292. In yet other preferred embodiments, the present invention provides a polynucleotide encoding a protein comprising the amino acid sequence of SEQ ID NO:192 from amino acid 75 to amino acid 107. In further preferred embodiments, the present invention provides a polynucleotide encoding a protein comprising a fragment of the amino acid sequence of SEQ ID NO:192 having biological activity, the fragment preferably comprising eight (more preferably twenty, most preferably thirty) contiguous amino acids of SEQ ID NO:192, or a polynucleotide encoding a protein comprising a fragment of the amino acid sequence of SEQ ID NO:192 having biological activity, the fragment comprising the amino acid sequence from amino acid 48 to amino acid 57 of SEQ ID NO:192.

Other embodiments provide the gene corresponding to the cDNA sequence of SEQ ID NO:191 and SEQ ID NO:193.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:191;
      (ab) SEQ ID NO:193, but excluding the poly(A) tail at the 3′ end of SEQ ID NO:193; and
      (ac) the nucleotide sequence of the cDNA insert of clone DF989_{—}3 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:191;
      (bb) SEQ ID NO:193, but excluding the poly(A) tail at the 3′ end of SEQ ID NO:193; and
      (bc) the nucleotide sequence of the cDNA insert of clone DF989_{—}3 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).

Preferably the polynucleotide isolated according to the above process comprises a nucleotide sequence corresponding to the cDNA sequences of SEQ ID NO:191 and SEQ ID NO:193, and extending contiguously from a nucleotide sequence corresponding to the 5′ end of SEQ ID NO:191 to a nucleotide sequence corresponding to the 3′ end of SEQ ID NO:193, but excluding the poly(A) tail at the 3′ end of SEQ ID NO:193. Also preferably the polynucleotide isolated according to the above process comprises a nucleotide sequence corresponding to the cDNA sequence of SEQ ID NO:191, and extending contiguously from a nucleotide sequence corresponding to the 5′ end of SEQ ID NO:191 to a nucleotide sequence corresponding to the 3′ end of SEQ ID NO:191. Also preferably the polynucleotide isolated according to the above process comprises a nucleotide sequence corresponding to the cDNA sequence of SEQ ID NO:191 from nucleotide 548 to nucleotide 868, and extending contiguously from a nucleotide sequence corresponding to the 5′ end of said sequence of SEQ ID NO:191 from nucleotide 548 to nucleotide 868, to a nucleotide sequence corresponding to the 3′ end of said sequence of SEQ ID NO:191 from nucleotide 548 to nucleotide 868. Also preferably the polynucleotide isolated according to the above process comprises a nucleotide sequence corresponding to the cDNA sequence of SEQ ID NO:191 from nucleotide 590 to nucleotide 868, and extending contiguously from a nucleotide sequence corresponding to the 5′ end of said sequence of SEQ ID NO:191 from nucleotide 590 to nucleotide 868, to a nucleotide sequence corresponding to the 3′ end of said sequence of SEQ ID NO:191 from nucleotide 590 to nucleotide 868.

In other embodiments, the present invention provides a composition comprising a protein, wherein said protein comprises an amino acid sequence selected from the group consisting of:
(a) the amino acid sequence of SEQ ID NO:192;
(b) the amino acid sequence of SEQ ID NO:192 from amino acid 75 to amino acid 107;
(c) a fragment of the amino acid sequence of SEQ ID NO:192, the fragment comprising eight contiguous amino acids of SEQ ID NO:192; and
(d) the amino acid sequence encoded by the cDNA insert of clone DF989_{—}3 deposited with the ATCC under accession number 98292;
   the protein being substantially free from other mammalian proteins. Preferably such protein comprises the amino acid sequence of SEQ ID NO:192 or the amino acid sequence of SEQ ID NO:192 from amino acid 75 to amino acid 107. In further preferred embodiments, the present invention provides a protein comprising a fragment of the amino acid sequence of SEQ ID NO:192 having biological activity, the fragment preferably comprising eight (more preferably twenty, most preferably thirty) contiguous amino adds of SEQ ID NO:192, or a protein comprising a fragment of the amino acid sequence of SEQ ID NO:192 having biological activity, the fragment comprising the amino acid sequence from amino acid 48 to amino acid 57 of SEQ ID NO:192.

In one embodiment, the present invention provides a composition comprising an isolated polynucleotide selected from the group consisting of:
(a) a polynucleotide comprising the nucleotide sequence of SEQ ID NO:194;
(b) a polynucleotide comprising the nucleotide sequence of SEQ ID NO:194 from nucleotide 251 to nucleotide 787;
(c) a polynucleotide comprising the nucleotide sequence of SEQ ID NO:194 from nucleotide 371 to nucleotide 787;
(d) a polynucleotide comprising the nucleotide sequence of the full-length protein coding sequence of clone DL162_{—}1 deposited with the ATCC under accession number 98292;
(e) a polynucleotide encoding the full-length protein encoded by the cDNA insert of clone DL162_{—}1 deposited with the ATCC under accession number 98292;
(f) a polynucleotide comprising the nucleotide sequence of a mature protein coding sequence of clone DL162_{—}1 deposited with the ATCC under accession number 98292;
(g) a polynucleotide encoding a mature protein encoded by the cDNA insert of clone DL162_{—}1 deposited with the ATCC under accession number 98292;
(h) a polynucleotide encoding a protein comprising the amino acid sequence of SEQ ID NO:195;
(i) a polynucleotide encoding a protein comprising a fragment of the amino acid sequence of SEQ ID NO:195 having biological activity, the fragment comprising eight contiguous amino acids of SEQ ID NO:195;
(j) a polynucleotide which is an allelic variant of a polynucleotide of (a>(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:194.

Preferably, such polynucleotide comprises the nucleotide sequence of SEQ ID NO:194 from nucleotide 251 to nucleotide 787; the nucleotide sequence of SEQ ID NO:194 from nucleotide 371 to nucleotide 787; the nucleotide sequence of the full-length protein coding sequence of clone DL162_{—}1 deposited with the ATCC under accession number 98292; or the nucleotide sequence of a mature protein coding sequence of clone DL162_{—}1 deposited with the ATCC under accession number 98292. In other preferred embodiments, the polynucleotide encodes the full-length or a mature protein encoded by the cDNA insert of clone DL162_{—}1 deposited with the ATCC under accession number 98292. In yet other preferred embodiments, the present invention provides a polynucleotide encoding a protein comprising the amino acid sequence of SEQ ID NO:195 from amino acid 38 to amino acid 170. In further preferred embodiments, the present invention provides a polynucleotide encoding a protein comprising a fragment of the amino acid sequence of SEQ ID NO:195 having biological activity, the fragment preferably comprising eight (more preferably twenty, most preferably thirty) contiguous amino acids of SEQ ID NO:195, or a polynucleotide encoding a protein comprising a fragment of the amino acid sequence of SEQ ID NO:195 having biological activity, the fragment comprising the amino acid sequence from amino acid 84 to amino acid 93 of SEQ ID NO:195.

Other embodiments provide the gene corresponding to the cDNA sequence of SEQ ID NO:194.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:194, but excluding the poly(A) tail at the 3′ end of SEQ ID NO:194; and
      (ab) the nucleotide sequence of the cDNA insert of clone DL162_{—}1 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:194, but excluding the poly(A) tail at the 3′ end of SEQ ID NO:194; and
      (bb) the nucleotide sequence of the cDNA insert of clone DL162_{—}1 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:194, and extending contiguously from a nucleotide sequence corresponding to the 5′ end of SEQ ID NO:194 to a nucleotide sequence corresponding to the 3′ end of SEQ ID NO:194, but excluding the poly(A) tail at the 3′ end of SEQ ID NO:194. Also preferably the polynucleotide isolated according to the above process comprises a nucleotide sequence corresponding to the cDNA sequence of SEQ ID NO:194 from nucleotide 251 to nucleotide 787, and extending contiguously from a nucleotide sequence corresponding to the 5′ end of said sequence of SEQ ID NO:194 from nucleotide 251 to nucleotide 787, to a nucleotide sequence corresponding to the 3′ end of said sequence of SEQ ID NO:194 from nucleotide 251 to nucleotide 787. Also preferably the polynucleotide isolated according to the above process comprises a nucleotide sequence corresponding to the cDNA sequence of SEQ ID NO:194 from nucleotide 371 to nucleotide 787, and extending contiguously from a nucleotide sequence corresponding to the 5′ end of said sequence of SEQ ID NO:194 from nucleotide 371 to nucleotide 787, to a nucleotide sequence corresponding to the 3′ end of said sequence of SEQ ID NO:194 from nucleotide 371 to nucleotide 787.

In other embodiments, the present invention provides a composition comprising a protein, wherein said protein comprises an amino acid sequence selected from the group consisting of:
(a) the amino acid sequence of SEQ ID NO:195;
(b) the amino acid sequence of SEQ ID NO:195 from amino acid 38 to amino acid 170;
(c) a fragment of the amino acid sequence of SEQ ID NO:195, the fragment comprising eight contiguous amino acids of SEQ ID NO:195; and
(d) the amino acid sequence encoded by the cDNA insert of clone DL162_{—}1 deposited with the ATCC under accession number 98292;
   the protein being substantially free from other mammalian proteins. Preferably such protein comprises the amino acid sequence of SEQ ID NO:195 or the amino acid sequence of SEQ ID NO:195 from amino acid 38 to amino acid 170. In further preferred embodiments, the present invention provides a protein comprising a fragment of the amino acid sequence of SEQ ID NO:195 having biological activity, the fragment preferably comprising eight (more preferably twenty, most preferably thirty) contiguous amino acids of SEQ ID NO:195, or a protein comprising a fragment of the amino acid sequence of SEQ ID NO:195 having biological activity, the fragment comprising the amino acid sequence from amino add 84 to amino acid 93 of SEQ ID NO:195.

In one embodiment, the present invention provides a composition comprising an isolated polynucleotide selected from the group consisting of:
(a) a polynucleotide comprising the nucleotide sequence of SEQ ID NO:196;
(b) a polynucleotide comprising the nucleotide sequence of SEQ ID NO:196 from nucleotide 121 to nucleotide 3345;
(c) a polynucleotide comprising the nucleotide sequence of SEQ ID NO:196 from nucleotide 160 to nucleotide 3345;
(d) a polynucleotide comprising the nucleotide sequence of SEQ ID NO:196 from nucleotide 2592 to nucleotide 3318;
(e) a polynucleotide comprising the nucleotide sequence of the full-length protein coding sequence of clone DL162_{—}2 deposited with the ATCC under accession number 98292;
(f) a polynucleotide encoding the full-length protein encoded by the cDNA insert of clone DL162_{—}2 deposited with the ATCC under accession number 98292;
(g) a polynucleotide comprising the nucleotide sequence of a mature protein coding sequence of clone DL162_{—}2 deposited with the ATCC under accession number 98292;
(h) a polynucleotide encoding a mature protein encoded by the cDNA insert of clone DL162_{—}2 deposited with the ATCC under accession number 98292;
(i) a polynucleotide encoding a protein comprising the amino acid sequence of SEQ ID NO:197;
(j) a polynucleotide encoding a protein comprising a fragment of the amino add sequence of SEQ ID NO:197 having biological activity, the fragment comprising eight contiguous amino acids of SEQ ID NO:197;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:196.

Preferably, such polynucleotide comprises the nucleotide sequence of SEQ ID NO:196 from nucleotide 121 to nucleotide 3345; the nucleotide sequence of SEQ ID NO:196 from nucleotide 160 to nucleotide 3345; the nucleotide sequence of SEQ ID NO:196 from nucleotide 2592 to nucleotide 3318; the nucleotide sequence of the full-length protein coding sequence of clone DL162_{—}2 deposited with the ATCC under accession number 98292; or the nucleotide sequence of a mature protein coding sequence of clone DL162_{—}2 deposited with the ATCC under accession number 98292. In other preferred embodiments, the polynucleotide encodes the full-length or a mature protein encoded by the cDNA insert of clone DL162_{—}2 deposited with the ATCC under accession number 98292. In yet other preferred embodiments, the present invention provides a polynucleotide encoding a protein comprising the amino add sequence of SEQ ID NO:197 from amino acid 860 to amino acid 1066. In further preferred embodiments, the present invention provides a polynucleotide encoding a protein comprising a fragment of the amino acid sequence of SEQ ID NO:197 having biological activity, the fragment preferably comprising eight (more preferably twenty, most preferably thirty) contiguous amino acids of SEQ ID NO:197, or a polynucleotide encoding a protein comprising a fragment of the amino acid sequence of SEQ ID NO:197 having biological activity, the fragment comprising the amino acid sequence from amino acid 532 to amino acid 541 of SEQ ID NO:197.

Other embodiments provide the gene corresponding to the cDNA sequence of SEQ ID NO:196.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:196, but excluding the poly(A) tail at the 3′ end of SEQ ID NO:196; and
      (ab) the nucleotide sequence of the cDNA insert of clone DL162_{—}2 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:196, but excluding the poly(A) tail at the 3′ end of SEQ ID NO:196; and
      (bb) the nucleotide sequence of the cDNA insert of clone DL162_{—}2 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
(iv) isolating the polynucleotide products of step (b)(iii).
   Preferably the polynucleotide isolated according to the above process comprises a nucleotide sequence corresponding to the cDNA sequence of SEQ ID NO:196, and extending contiguously from a nucleotide sequence corresponding to the 5′ end of SEQ ID NO:196 to a nucleotide sequence corresponding to the 3′ end of SEQ ID NO:196, but excluding the poly(A) tail at the 3′ end of SEQ ID NO:196. Also preferably the polynucleotide isolated according to the above process comprises a nucleotide sequence corresponding to the cDNA sequence of SEQ ID NO:196 from nucleotide 121 to nucleotide 3345, and extending contiguously from a nucleotide sequence corresponding to the 5′ end of said sequence of SEQ ID NO:196 from nucleotide 121 to nucleotide 3345, to a nucleotide sequence corresponding to the 3′ end of said sequence of SEQ ID NO:196 from nucleotide 121 to nucleotide 3345. Also preferably the polynucleotide isolated according to the above process comprises a nucleotide sequence corresponding to the cDNA sequence of SEQ ID NO:196 from nucleotide 160 to nucleotide 3345, and extending contiguously from a nucleotide sequence corresponding to the 5′ end of said sequence of SEQ ID NO:196 from nucleotide 160 to nucleotide 3345, to a nucleotide sequence corresponding to the 3′ end of said sequence of SEQ ID NO:196 from nucleotide 160 to nucleotide 3345. Also preferably the polynucleotide isolated according to the above process comprises a nucleotide sequence corresponding to the cDNA sequence of SEQ ID NO:196 from nucleotide 2592 to nucleotide 3318, and extending contiguously from a nucleotide sequence corresponding to the 5′ end of said sequence of SEQ ID NO:196 from nucleotide 2592 to nucleotide 3318, to a nucleotide sequence corresponding to the 3′ end of said sequence of SEQ ID NO:196 from nucleotide 2592 to nucleotide 3318.

In other embodiments, the present invention provides a composition comprising a protein, wherein said protein comprises an amino acid sequence selected from the group consisting of:
(a) the amino acid sequence of SEQ ID NO:197;
(b) the amino acid sequence of SEQ ID NO:197 from amino acid 860 to amino acid 1066;
(c) a fragment of the amino acid sequence of SEQ ID NO:197, the fragment comprising eight contiguous amino acids of SEQ ID NO:197; and
(d) the amino acid sequence encoded by the cDNA insert of clone DL162_{—}2 deposited with the ATCC under accession number 98292;
   the protein being substantially free from other mammalian proteins. Preferably such protein comprises the amino acid sequence of SEQ ID NO:197 or the amino acid sequence of SEQ ID NO:197 from amino acid 860 to amino acid 1066. In further preferred embodiments, the present invention provides a protein comprising a fragment of the amino acid sequence of SEQ ID NO:197 having biological activity, the fragment preferably comprising eight (more preferably twenty, most preferably thirty) contiguous amino acids of SEQ ID NO:197, or a protein comprising a fragment of the amino acid sequence of SEQ ID NO:197 having biological activity, the fragment comprising the amino acid sequence from amino acid 532 to amino acid 541 of SEQ ID NO:197.

In one embodiment, the present invention provides a composition comprising an isolated polynucleotide selected from the group consisting of:
(a) a polynucleotide comprising the nucleotide sequence of SEQ ID NO:198;
(b) a polynucleotide comprising the nucleotide sequence of SEQ ID NO:198 from nucleotide 117 to nucleotide 2600;
(c) a polynucleotide comprising the nucleotide sequence of SEQ ID NO:198 from nucleotide 2130 to nucleotide 2600;
(d) a polynucleotide comprising the nucleotide sequence of SEQ ID NO:198 from nucleotide 1 to nucleotide 506;
(e) a polynucleotide comprising the nucleotide sequence of the full-length protein coding sequence of clone EC172_{—}1 deposited with the ATCC under accession number 98292;
(f) a polynucleotide encoding the full-length protein encoded by the cDNA insert of clone EC172_{—}1 deposited with the ATCC under accession number 98292;
(g) a polynucleotide comprising the nucleotide sequence of a mature protein coding sequence of clone EC172_{—}1 deposited with the ATCC under accession number 98292;
(h) a polynucleotide encoding a mature protein encoded by the cDNA insert of clone EC172_{—}1 deposited with the ATCC under accession number 98292;
(i) a polynucleotide encoding a protein comprising the amino acid sequence of SEQ ID NO:199;
(j) a polynucleotide encoding a protein comprising a fragment of the amino acid sequence of SEQ ID NO:199 having biological activity, the fragment comprising eight contiguous amino acids of SEQ ID NO:199;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:198.

Preferably, such polynucleotide comprises the nucleotide sequence of SEQ ID NO:198 from nucleotide 117 to nucleotide 2600; the nucleotide sequence of SEQ ID NO:198 from nucleotide 2130 to nucleotide 2600; the nucleotide sequence of SEQ ID NO:198 from nucleotide 1 to nucleotide 506; the nucleotide sequence of the full-length protein coding sequence of clone EC172_{—}1 deposited with the ATCC under accession number 98292; or the nucleotide sequence of a mature protein coding sequence of clone EC172_{—}1 deposited with the ATCC under accession number 98292. In other preferred embodiments, the polynucleotide encodes the full-length or a mature protein encoded by the cDNA insert of clone EC172_{—}1 deposited with the ATCC under accession number 98292. In yet other preferred embodiments, the present invention provides a polynucleotide encoding a protein comprising the amino acid sequence of SEQ ID NO:199 from amino acid 1 to amino acid 130. In further preferred embodiments, the present invention provides a polynucleotide encoding a protein comprising a fragment of the amino add sequence of SEQ ID NO:199 having biological activity, the fragment preferably comprising eight (more preferably twenty, most preferably thirty) contiguous amino acids of SEQ ID NO:199, or a polynucleotide encoding a protein comprising a fragment of the amino acid sequence of SEQ ID NO:199 having biological activity, the fragment comprising the amino acid sequence from amino acid 409 to amino acid 418 of SEQ ID NO:199.

Other embodiments provide the gene corresponding to the cDNA sequence of SEQ ID NO:198.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:198, but excluding the poly(A) tail at the 3′ end of SEQ ID NO:198; and
      (ab) the nucleotide sequence of the cDNA insert of clone EC172_{—}1 deposited with the ATCC under accession number 98292;
   (ii) hybridizing said probe(s) to human genomic DNA in conditions at least as stringent as 4×SSC at 50 degrees C.; and
   (iii) isolating the DNA polynucleotides detected with the probe(s); and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:198, but excluding the poly(A) tail at the 3′ end of SEQ ID NO:198; and
      (bb) the nucleotide sequence of the cDNA insert of clone EC172_{—}1 deposited with the ATCC under accession number 98292;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:198, and extending contiguously from a nucleotide sequence corresponding to the 5′ end of SEQ ID NO:198 to a nucleotide sequence corresponding to the 3′ end of SEQ ID NO:198, but excluding the poly(A) tail at the 3′ end of SEQ ID NO:198. Also preferably the polynucleotide isolated according to the above process comprises a nucleotide sequence corresponding to the cDNA sequence of SEQ ID NO:198 from nucleotide 117 to nucleotide 2600, and extending contiguously from a nucleotide sequence corresponding to the 5′ end of said sequence of SEQ ID NO:198 from nucleotide 117 to nucleotide 2600, to a nucleotide sequence corresponding to the 3′ end of said sequence of SEQ ID NO:198 from nucleotide 117 to nucleotide 2600. Also preferably the polynucleotide isolated according to the above process comprises a nucleotide sequence corresponding to the cDNA sequence of SEQ ID NO:198 from nucleotide 2130 to nucleotide 2600, and extending contiguously from a nucleotide sequence corresponding to the 5′ end of said sequence of SEQ ID NO:198 from nucleotide 2130 to nucleotide 2600, to a nucleotide sequence corresponding to the 3′ end of said sequence of SEQ ID NO:198 from nucleotide 2130 to nucleotide 2600. Also preferably the polynucleotide isolated according to the above process comprises a nucleotide sequence corresponding to the cDNA sequence of SEQ ID NO:198 from nucleotide 1 to nucleotide 506, and extending contiguously from a nucleotide sequence corresponding to the 5′ end of said sequence of SEQ ID NO:198 from nucleotide 1 to nucleotide 506, to a nucleotide sequence corresponding to the 3′ end of said sequence of SEQ ID NO:198 from nucleotide 1 to nucleotide 506.

In other embodiments, the present invention provides a composition comprising a protein, wherein said protein comprises an amino acid sequence selected from the group consisting of:
(a) the amino acid sequence of SEQ ID NO:199;
(b) the amino acid sequence of SEQ ID NO:199 from amino acid 1 to amino acid 130;
(c) a fragment of the amino acid sequence of SEQ ID NO:199, the fragment comprising eight contiguous amino acids of SEQ ID NO:199; and
(d) the amino acid sequence encoded by the cDNA insert of clone EC172_{—}1 deposited with the ATCC under accession number 98292;
   the protein being substantially free from other mammalian proteins. Preferably such protein comprises the amino acid sequence of SEQ ID NO:199 or the amino acid sequence of SEQ ID NO:199 from amino acid 1 to amino acid 130. In further preferred embodiments, the present invention provides a protein comprising a fragment of the amino acid sequence of SEQ ID NO:199 having biological activity, the fragment preferably comprising eight (more preferably twenty, most preferably thirty) contiguous amino acids of SEQ ID NO:199, or a protein comprising a fragment of the amino acid sequence of SEQ ID NO:199 having biological activity, the fragment comprising the amino acid sequence from amino acid 409 to amino acid 418 of SEQ ID NO:199.

In certain preferred embodiments, the polynucleotide is operably linked to an expression control sequence. The invention also provides a host cell, including bacterial, yeast, insect and mammalian cells, transformed with such polynucleotide compositions. Also provided by the present invention are organisms that have enhanced, reduced, or modified expression of the gene(s) corresponding to the polynucleotide sequences disclosed herein.

Processes are also provided for producing a protein, which comprise:
(a) growing a culture of the host cell transformed with such polynucleotide compositions in a suitable culture medium; and
(b) purifying the protein from the culture.
   The protein produced according to such methods is also provided by the present invention.

Protein compositions of the present invention may further comprise a pharmaceutically acceptable carrier. Compositions comprising an antibody which specifically reacts with such protein are also provided by the present invention.

Methods are also provided for preventing, treating or ameliorating a medical condition which comprises administering to a mammalian subject a therapeutically effective amount of a composition comprising a protein of the present invention and a pharmaceutically acceptable carrier.

### BRIEF DESCRIPTION OF THE DRAWINGS

FIGS. 1A and 1B are schematic representations of the pED6 and pNOTs vectors, respectively, used for deposit of clones disclosed herein.

### DETAILED DESCRIPTION

Isolated Proteins and Polynucleotides

Nucleotide and amino acid sequences, as presently determined, are reported below for each clone and protein disclosed in the present application. The nucleotide sequence of each clone can readily be determined by sequencing of the deposited clone in accordance with known methods. The predicted amino acid sequence (both full-length and mature forms) can then be determined from such nucleotide sequence. The amino acid sequence of the protein encoded by a particular clone can also be determined by expression of the clone in a suitable host cell, collecting the protein and determining its sequence. For each disclosed protein applicants have identified what they have determined to be the reading frame best identifiable with sequence information available at the time of filing.

As used herein a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

Clone “AX65_{—}22”

A polynucleotide of the present invention has been identified as clone “AX65_{—}22”.

AX65_{—}22 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AX65_{—}22 is a full-length clone, including the entire coding sequence of a secreted protein (also referred to herein as “AX65_{—}22 protein”).

The nucleotide sequence of the 5′ portion of AX65_{—}22 as presently determined is reported in SEQ ID NO:1. What applicants presently believe is the proper reading frame for the coding region is indicated in SEQ ID NO:2. The predicted amino acid sequence of the AX65_{—}22 protein corresponding to the foregoing nucleotide sequence is reported in SEQ ID NO:2. Amino acids 8 to 20 of SEQ ID NO:2 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the AX65_{—}22 protein. Additional nucleotide sequence from the 3′ portion of AX65_{—}22, including a poly(A) tail, is reported in SEQ ID NO:3.

The EcoRI/NotI restriction fragment obtainable from the deposit containing clone AX65_{—}22 should be approximately 3500 bp. The nucleotide sequence disclosed herein for AX65_{—}22 was searched against the GenBank and GeneSeq nucleotide sequence databases using BLASTN/BLASTX and FASTA search protocols. AX65_{—}22 demonstrated at least some similarity with sequences identified as T08476 (Eukaryotic expression vector pAPEX-3p) and U46493 (Cloning vector pFlp recombinase gene, complete cds). The predicted AX65_{—}22 protein demonstrated at least some homology with sequences identified as J01969 (DNA polymerase [Human adenovirus type 5]), R07640 (Deduced protein sequence of p170-2 comprising T4), and X57205 (fibroblast growth factor receptor [Homo sapiens]). Based upon sequence similarity, AX65_{—}22 proteins and each similar protein or peptide may share at least some activity.

Clone “BD335 14”

A polynucleotide of the present invention has been identified as clone “BD335_{—}14”. BD335_{—}14 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD335_{—}14 is a full-length done, including the entire coding sequence of a secreted protein (also referred to herein as “BD335_{—}14 protein”).

The nucleotide sequence of BD335_{—}14 as presently determined is reported in SEQ ID NO:4, and includes a poly(A) tail. What applicants presently believe to be the proper reading frame and the predicted amino acid sequence of the BD335_{—}14 protein corresponding to the foregoing nucleotide sequence is reported in SEQ ID NO:5.

The EcoRI/NotI restriction fragment obtainable from the deposit containing clone BD335_{—}14 should be approximately 3000 bp.

The nucleotide sequence disclosed herein for BD335_{—}14 was searched against the GenBank and GeneSeq nucleotide sequence databases using BLASTN/BLASTX and FASTA search protocols. The predicted BD335_{—}14 protein demonstrated at least some similarity with sequences identified as U83511 (APXL [Homo sapiens]). Based upon sequence similarity, BD335_{—}14 proteins and each homologous protein or peptide may share at least some activity. The TopPredII computer program predicts three potential transmembrane domains within the BD335_{—}14 protein sequence, one centered around amino add 80, another around amino acid 320, and a third around amino acid 700 of SEQ ID NO:5.

Clone “BG241_{—}1”

A polynucleotide of the present invention has been identified as clone “BG241_{—}1”. BG241_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG241_{—}1 is a full-length done, including the entire coding sequence of a secreted protein (also referred to herein as “BG241_{—}1 protein”). The nucleotide sequence of the 5′ portion of BG241_{—}1 as presently determined is reported in SEQ ID NO:6. An additional internal nucleotide sequence from BG241_{—}1 as presently determined is reported in SEQ ID NO:7. What applicants believe is the proper reading frame and the predicted amino acid sequence encoded by such internal sequence is reported in SEQ ID NO:8. Additional nucleotide sequence from the 3′ portion of BG241_{—}1, including a poly(A) tail, is reported in SEQ ID NO:9.

The EcoRI/NotI restriction fragment obtainable from the deposit containing clone BG241_{—}1 should be approximately 800 bp.

The nucleotide sequence disclosed herein for BG241_{—}1 was searched against the GenBank and GeneSeq nucleotide sequence databases using BLASTN/BLASTX and FASTA search protocols. BG241_{—}1 demonstrated at least some similarity with sequences identified as AI082187 (ox75f01.x1×1 NhHMPu_S1 Homo sapiens cDNA clone IMAGE 1662169 3′ similar to contains element MSR1 repetitive element; mRNA sequence), W38781 (zb27g08.r1 Soares parathyroid tumor NbHPA Homo sapiens), and Y12781 (Homo sapiens mRNA for transducin. (beta) like 1 protein). The predicted BG241_{—}1 protein demonstrated at least some similarity to sequences identified as Y12781 (transducin (beta) like 1 protein [Homo sapiens]) and other beta-transducin-like proteins (see GenBank accession numbers L28125 and T86738). Based upon sequence similarity, BG241_{—}1 proteins and each similar protein or peptide may share at least some activity.

Clone “BL187_{—}4”

A polynucleotide of the present invention has been identified as clone “BL187_{—}4”. BL187_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL187_{—}4 is a full-length clone, including the entire coding sequence of a secreted protein (also referred to herein as “BL187_{—}4 protein”).

The nucleotide sequence of BL187_{—}4 as presently determined is reported in SEQ ID NO:10, and includes a poly(A) tail. What applicants presently believe to be the proper reading frame and the predicted amino acid sequence of the BL187_{—}4 protein corresponding to the foregoing nucleotide sequence is reported in SEQ ID NO:11. Amino acids 17 to 29 of SEQ ID NO:11 are a predicted leader/signal sequence, with the predicted mature amino acid sequence beginning at amino acid 30. Due to the hydrophobic nature of the predicted leader/signal sequence, it is likely to act as a transmembrane domain should the predicted leader/signal sequence not be separated from the remainder of the BL187_{—}4 protein.

The EcoRI/NotI restriction fragment obtainable from the deposit containing clone BL187_{—}4 should be approximately 2300 bp.

The nucleotide sequence disclosed herein for BL187_{—}4 was searched against the GenBank and GeneSeq nucleotide sequence databases using BLASTN/BLASTX and FASTA search protocols. BL187_{—}4 demonstrated at least some similarity with sequences identified as AA476210 (zw35g01.s1 Soares ovary tumor NbHOT Homo sapiens cDNA clone 771312 3′, mRNA sequence), AA868505 (ak43b04.s1 Soares testis NHT Homo sapiens cDNA clone IMAGE 1408687 3′ similar to SW PLZF_HUMAN Q05516 ZINC FINGER PROTEIN PLZF; mRNA sequence), AA927876 (om18b09.s1 Soares NFL T GBC S1 Homo sapiens cDNA clone IMAGE:1541369 3′, mRNA sequence), AD000671 (Homo sapiens DNA from chromosome 19-cosmid f24109 containing HRX2, genomic sequence), H48938 (EST0010 Homo sapiens cDNA clone HTN-6-15), and Z63958 (H. sapiens CpG DNA, clone 93d10, forward read cpg93d10.ft1a). The predicted amino acid sequence disclosed herein for BL187_{—}4 was searched against the GenPept and GeneSeq amino acid sequence databases using the BLASTX search protocol. The predicted BL1874 protein demonstrated at least some similarity to sequences identified as R95242 (HIC-1 polypeptide), Z19002 (kruppel-like zinc finger protein [Homo sapiens]), and a number of other zinc-finger proteins. Based upon sequence similarity, BL187_{—}4 proteins and each similar protein or peptide may share at least some activity. Motifs analysis indicates the presence of two zinc-finger (C2H2 type) domains centered around amino acids 375 and 430 of SEQ ID NO:11, respectively. The TopPredII computer program predicts two potential transmembrane domains within the BL187_{—}4 protein sequence, one centered around amino acid 30 and another around amino acid 260 of SEQ ID NO:11. BL187_{—}4 protein appears to be a novel secreted or membrane-associated zinc-finger protein.

Clone “BL249_{—}18”

A polynucleotide of the present invention has been identified as clone “BL249_{—}18”. BL249_{—}18 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL249_{—}18 is a full-length clone, including the entire coding sequence of a secreted protein (also referred to herein as “BL249_{—}18 protein”).

The nucleotide sequence of BL249_{—}18 as presently determined is reported in SEQ ID NO:12, and includes a poly(A) tail. What applicants presently believe to be the proper reading frame and the predicted amino acid sequence of the BL249_{—}18 protein corresponding to the foregoing nucleotide sequence is reported in SEQ ID NO:13. Amino acids 32 to 44 of SEQ ID NO:13 are a predicted leader/signal sequence, with the predicted mature amino acid sequence beginning at amino acid 45. Due to the hydrophobic nature of the predicted leader/signal sequence, it is likely to act as a transmembrane domain should the predicted leader/signal sequence not be separated from the remainder of the BL249_{—}18 protein.

The EcoRI/NotI restriction fragment obtainable from the deposit containing clone BL249_{—}18 should be approximately 2300 bp.

The nucleotide sequence disclosed herein for BL249_{—}18 was searched against the GenBank and GeneSeq nucleotide sequence databases using BLASTN/BLASTX and FASTA search protocols. BL249_{—}18 demonstrated at least some similarity with sequences identified as AA034864 (mi53f01.r1 Soares mouse embryo NbME13.5 14.5 Mus musculus cDNA clone 467257 5′), AA115100 (z102h12.s1 Soares pregnant uterus NbHPU Homo sapiens cDNA clone 491207 3′), AA219365 (zr04c06.r1 Stratagene NT2 neuronal precursor 937230 Homo sapiens cDNA clone 650506 5′), AA399095 (zt59b06.r1 Soares testis NHT Homo sapiens cDNA clone 726611 5′), R82633 (yj20a05.s1 Homo sapiens cDNA clone 149264 3′), and T22047 (Human gene signature HUMGS03590). The predicted amino acid sequence disclosed herein for BL249_{—}18 was searched against the GenPept and GeneSeq amino acid sequence databases using the BLASTX search protocol. The predicted BL249_{—}18 protein demonstrated at least some similarity to sequences identified as AC003673 (unknown protein (AAC09020.1) [Arabidopsis thaliana]) and Z98598 (hypothetical protein [Schizosaccharomyces pombe]). Based upon sequence similarity, BL249_{—}18 proteins and each similar protein or peptide may share at least some activity. The TopPredII computer program predicts two potential transmembrane domains within the BL249_{—}18 protein sequence, one centered around amino acid 45 and another around amino acid 680 of SEQ ID NO:13.

Clone “BO71_{—}1”

A polynucleotide of the present invention has been identified as clone “BO71_{—}1”. BO071_{—}1 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BO71_{—}1 is a full-length done, including the entire coding sequence of a secreted protein (also referred to herein as “BO71_{—}1 protein”).

The nucleotide sequence of the 5′ portion of BO71_{—}1 as presently determined is reported in SEQ ID NO:14. An additional internal nucleotide sequence from BO71_{—}1 as presently determined is reported in SEQ ID NO:15. What applicants believe is the proper reading frame and the predicted amino acid sequence encoded by such internal sequence is reported in SEQ ID NO:16. Additional nucleotide sequence from the 3′ portion of BO71_{—}1, including a poly(A) tail, is reported in SEQ ID NO:17.

The EcoRI/NotI restriction fragment obtainable from the deposit containing clone BO71_{—}1 should be approximately 2000 bp.

The nucleotide sequence disclosed herein for BO71_{—}1 was searched against the GenBank and GeneSeq nucleotide sequence databases using BLASTN/BLASTX and FASTA search protocols. BO71_{—}1 demonstrated at least some similarity with sequences identified as X86809 (H. sapiens mRNA for major astrocytic phosphoprotein PEA-15).

The predicted amino acid sequence disclosed herein for BO71_{—}1 was searched against the GenPept and GeneSeq amino acid sequence databases using the BLASTX search protocol. The predicted BO71_{—}1 protein demonstrated at least some similarity to sequences identified as U06144 (cellular disintegrin-related protein [Mus musculus]). Based upon sequence similarity, BO71_{—}1 proteins and each similar protein or peptide may share at least some activity.

Clone “BO365_{—}2”

A polynucleotide of the present invention has been identified as clone “BO365_{—}2”.

BO365_{—}2 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BO365_{—}2 is a full-length done, including the entire coding sequence of a secreted protein (also referred to herein as “BO365_{—}2 protein”).

The nucleotide sequence of BO365_{—}2 as presently determined is reported in SEQ ID NO:18, and includes a poly(A) tail. What applicants presently believe to be the proper reading frame and the predicted amino acid sequence of the BO365_{—}2 protein corresponding to the foregoing nucleotide sequence is reported in SEQ ID NO:19.

The EcoRI/NotI restriction fragment obtainable from the deposit containing clone BO365_{—}2 should be approximately 2800 bp.

The nucleotide sequence disclosed herein for BO365_{—}2 was searched against the GenBank and GeneSeq nucleotide sequence databases using BLASTN/BLASTX and FASTA search protocols. BO365_{—}2 demonstrated at least some similarity with sequences identified as D63876 (Human mRNA for KIAA0154 gene, partial cds) and Z83844 (Human DNA sequence *** SEQUENCING IN PROGRESS *** from clone 37E16; HTGS phase 1). The predicted amino acid sequence disclosed herein for BO365_{—}2 was searched against the GenPept and GeneSeq amino acid sequence databases using the BLASTX search protocol. The predicted BO365_{—}2 protein demonstrated at least some similarity to sequences identified as D10250 (alpha-fetoprotein enhancer binding protein [Homo sapiens]), D63876 (KIAA0154 gene product is related to mouse gamma adaptin [Homo sapiens]), and R23962 (AFP-1). Based upon sequence similarity, BO365_{—}2 proteins and each similar protein or peptide may share at least some activity. The TopPredII computer program predicts three potential transmembrane domains within the BO365_{—}2 protein sequence, centered around amino acids 70, 140, and 180 of SEQ ID NO:19, respectively.

Clone “BV51_{—}1”

A polynucleotide of the present invention has been identified as clone “BV51_{—}1”. BV51_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV51_{—}1 is a full-length clone, including the entire coding sequence of a secreted protein (also referred to herein as “BV51.1 protein”).

The nucleotide sequence of the 5′ portion of BV51_{—}1 as presently determined is reported in SEQ ID NO:20. What applicants presently believe is the proper reading frame for the coding region is indicated in SEQ ID NO:21. The predicted amino acid sequence of the BV51_{—}1 protein corresponding to the foregoing nucleotide sequence is reported in SEQ ID NO:21. Additional nucleotide sequence from the 3′ portion of BV51_{—}1, including a poly(A) tail, is reported in SEQ ID NO:22.

The EcoRI/NotI restriction fragment obtainable from the deposit containing clone BV51_{—}1 should be approximately 970 bp.

The nucleotide sequence disclosed herein for BV51_{—}1 was searched against the GenBank and GeneSeq nucleotide sequence databases using BLASTN/BLASTX and FASTA search protocols. BV51_{—}1 demonstrated at least some similarity with sequences identified as AB012130 (Homo sapiens SBC2 mRNA for sodium bicarbonate cotransporter2, complete cds) and U46493 (Cloning vector pFlp recombinase gene, complete cds). The predicted amino acid sequence disclosed herein for BV51_{—}1 was searched against the GenPept and GeneSeq amino acid sequence databases using the BLASTX search protocol. The predicted BV51_{—}1 protein demonstrated at least some similarity to sequences identified as AB01213 (sodium bicarbonate cotransporter2 [Homo sapiens]). Based upon sequence similarity, BV51_{—}1 proteins and each similar protein or peptide may share at least some activity.

Clone “BV140_{—}3”

A polynucleotide of the present invention has been identified as clone “BV140_{—}3”. BV140_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV140_{—}3 is a full-length done, including the entire coding sequence of a secreted protein (also referred to herein as “BV140_{—}3 protein”).

The nucleotide sequence of the 5′ portion of BV140_{—}3 as presently determined is reported in SEQ ID NO:23. An additional internal nucleotide sequence from BV140_{—}3 as presently determined is reported in SEQ ID NO:24. What applicants believe is the proper reading frame and the predicted amino acid sequence encoded by such internal sequence is reported in SEQ ID NO:25. Additional nucleotide sequence from the 3′ portion of BV140_{—}3, including a poly(A) tail, is reported in SEQ ID NO:26.

The EcoRI/NotI restriction fragment obtainable from the deposit containing clone BV140_{—}3 should be approximately 3500 bp.

The nucleotide sequence disclosed herein for BV140_{—}3 was searched against the GenBank and GeneSeq nucleotide sequence databases using BLASTN/BLASTX and FASTA search protocols. BV140_{—}3 demonstrated at least some similarity with sequences identified as H72799 (yu07d10.r1 Homo sapiens cDNA clone 233107 5′) and T94057 (ye33g08.r1 Homo sapiens cDNA clone 119582 5′. Based upon sequence similarity, BV140_{—}3 proteins and each similar protein or peptide may share at least some activity.

Clone “BV141_{—}2”

A polynucleotide of the present invention has been identified as clone “BV141_{—}2”. BV141_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV141_{—}2 is a full-length clone, including the entire coding sequence of a secreted protein (also referred to herein as “BV141_{—}2 protein”).

The nucleotide sequence of BV141_{—}2 as presently determined is reported in SEQ ID NO:27, and includes a poly(A) tail. What applicants presently believe to be the proper reading frame and the predicted amino acid sequence of the BV141_{—}2 protein corresponding to the foregoing nucleotide sequence is reported in SEQ ID NO:28.

The EcoRI/NotI restriction fragment obtainable from the deposit containing clone BV141_{—}2 should be approximately 1100 bp.

The nucleotide sequence disclosed herein for BV141_{—}2 was searched against the GenBank and GeneSeq nucleotide sequence databases using BLASTN/BLASTX and FASTA search protocols. BV141_{—}2 demonstrated at least some similarity with sequences identified as L26860 (Mus musculus (C6e) heavy chain immunoglobulin variable region gene). Based upon sequence similarity, BV141_{—}2 proteins and each similar protein or peptide may share at least some activity. The TopPredII computer program predicts two potential transmembrane domains within the BV141_{—}2 protein sequence, one centered around amino acid 34 and another around amino acid 65 of SEQ ID NO:28. The nucleotide sequence of BV141_{—}2 indicates that it may contain one or more of the following repetitive element(s): L1 repeat

Clone “CC194_{—}4”

A polynucleotide of the present invention has been identified as clone “CC194_{—}4”. CC194_{—}4 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC194_{—}4 is a full-length clone, including the entire coding sequence of a secreted protein (also referred to herein as “CC194_{—}4 protein”).

The nucleotide sequence of the 5′ portion of CC194_{—}4 as presently determined is reported in SEQ ID NO:29. What applicants presently believe is the proper reading frame for the coding region is indicated in SEQ ID NO:30. The predicted amino acid sequence of the CC194_{—}4 protein corresponding to the foregoing nucleotide sequence is reported in SEQ ID NO:30. Amino acids 88 to 100 of SEQ ID NO:30 are a predicted leader/signal sequence, with the predicted mature amino acid sequence beginning at amino acid 101. Due to the hydrophobic nature of the predicted leader/signal sequence, it is likely to act as a transmembrane domain should the predicted leader/signal sequence not be separated from the remainder of the CC194_{—}4 protein. Additional nucleotide sequence from the 3′ portion of CC194_{—}4, including a poly(A) tail, is reported in SEQ ID NO:31.

The EcoRI/NotI restriction fragment obtainable from the deposit containing clone CC194_{—}4 should be approximately 3300 bp.

The nucleotide sequence disclosed herein for CC194_{—}4 was searched against the GenBank and GeneSeq nucleotide sequence databases using BLASTN/BLASTX and FASTA search protocols. CC194_{—}4 demonstrated at least some similarity with sequences identified as AA722214 (zh20f10.s1 Soares pineal gland N3HPG Homo sapiens cDNA clone 412651 3′, mRNA sequence), H11476 (ym10h08.s1 Homo sapiens cDNA clone 47781 3′), H111581 (ym10h08.r1 Soares infant brain 1NIB Homo sapiens cDNA clone IMAGE:47781 5′ similar to SP:C36E8.3 CE00911; mRNA sequence), H23044 (ym51d07.r1 Homo sapiens cDNA clone 52058 5′ similar to SP:C36E8.3 CE00911), N93789 (zb64g05.s1 Soares fetal lung NbHL19W Homo sapiens cDNA clone 308408 3), and W54544 (mc99a01.r1 Soares mouse embryo NbME13.5 14.5 Mus musculus). The predicted amino acid sequence disclosed herein for CC194_{—}4 was searched against the GenPept and GeneSeq amino acid sequence databases using the BLASTX search protocol. The predicted CC194_{—}4 protein demonstrated at least some similarity to sequences identified as M38561 (CAD [Homo sapiens]). Based upon sequence similarity, CC194_{—}4 proteins and each similar protein or peptide may share at least some activity.

Clone “DA136_{—}11”

A polynucleotide of the present invention has been identified as clone “DA136_{—}11”. DA136_{—}11 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. DA136_{—}11 includes at least a portion of the coding sequence of a secreted protein (also referred to herein as “DA136_{—}11 protein”).

The nucleotide sequence of DA136_{—}11 as presently determined is reported in SEQ ID NO:32, and includes a poly(A) tail. What applicants presently believe to be the proper reading frame and the predicted amino acid sequence of the DA136_{—}11 protein corresponding to the foregoing nucleotide sequence is reported in SEQ ID NO:33.

The EcoRI/NotI restriction fragment obtainable from the deposit containing clone DA136_{—}11 should be approximately 3800 bp. The nucleotide sequence disclosed herein for DA136_{—}11 was searched against the GenBank and GeneSeq nucleotide sequence databases using BLASTN/BLASTX and FASTA search protocols. DA136_{—}11 demonstrated at least some similarity with sequences identified as AA523414 (ng30a07.s1 NCl_CGAP_Co3 Homo sapiens cDNA clone 936276), H89334 (yw25h09.r1 Homo sapiens cDNA clone 253313 5′), R59925 (yh11b12.s1 Homo sapiens cDNA clone 42891 3), T66165 (Human interleukin-12 receptor alpha chain NR4 DNA), Y09328 (H. sapiens mRNA for IL13 receptor alpha-1 chain), and Y10659 (H. sapiens IL-13Ra mRNA). The predicted amino acid sequence disclosed herein for DA136_{—}11 was searched against the GenPept and GeneSeq amino acid sequence databases using the BLASTX search protocol. The predicted DA136_{—}11 protein demonstrated at least some similarity to sequences identified as L08960 (cell adhesion molecule [Gallus gallus]), M34083 (lactogen receptor precursor [Rattus norvegicus]), M59941 (GM-CSF receptor beta chain [Homo sapiens]), WO9822 (Human interleukin-12 receptor alpha chain NR4), X61178 (interleukin-5 receptor type 3 [Homo sapiens]), and Y10659 (L-13Ra [Homo sapiens]). Based upon sequence similarity, DA136_{—}11 proteins and each similar protein or peptide may share at least some activity. The TopPredII computer program predicts a potential transmembrane domain within the DA136_{—}11 protein sequence; centered around amino acid 215 of SEQ ID NO:33.

Clone “AR415_{—}4”

A polynucleotide of the present invention has been identified as clone “AR415_{—}4”. AR415_{—}4 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AR415_{—}4 is a full-length clone, including the entire coding sequence of a secreted protein (also referred to herein as “AR415_{—}4 protein”).

The nucleotide sequence of AR415_{—}4 as presently determined is reported in SEQ ID NO:34, and includes a poly(A) tail. What applicants presently believe to be the proper reading frame and the predicted amino acid sequence of the AR415_{—}4 protein corresponding to the foregoing nucleotide sequence is reported in SEQ ID NO:35. Amino acids 14 to 26 of SEQ ID NO:35 are a predicted leader/signal sequence, with the predicted mature amino acid sequence beginning at amino acid 27. Due to the hydrophobic nature of the predicted leader/signal sequence, it is likely to act as a transmembrane domain should the predicted leader/signal sequence not be separated from the remainder of the AR415_{—}4 protein.

The EcoRI/NotI restriction fragment obtainable from the deposit containing clone AR415_{—}4 should be approximately 1500 bp. The nucleotide sequence disclosed herein for AR415_{—}4 was searched against the GenBank and GeneSeq nucleotide sequence databases using BLASTN/BLASTX and FASTA search protocols. AR415_{—}4 demonstrated at least some similarity with sequences identified as AA100799 (zm26d01.s1 Stratagene pancreas (#937208) Homo sapiens cDNA clone 526753 3′), AA100852 (zm26d01.r1 Stratagene pancreas (#937208) Homo sapiens cDNA clone 526753 5′ similar to SW C002_HUMAN P19075 TUMOR-ASSOCIATED ANTIGEN CO-029), AA146605 (zo35c09.r1 Stratagene colon (#937204) Homo sapiens cDNA clone 588880 5′ similar to SW:CO02_HUMAN P19075 TUMOR-ASSOCIATED ANTIGEN CO-029), AA224847 (nc33c12.s1 NCI CGAP Pr2 Homo sapiens cDNA clone 4079 similar to SW:CO02_HUMAN P9075 TUMOR-ASSOCIATED ANTIGEN CO-029), AA225191 (nc21h08.s1 Na CGAP Pr1 Homo sapiens cDNA clone 2968), AA593864 (nn19f08.s1 NCI_CGAP_Co12 Homo sapiens cDNA clone IMAGE:1084359), D26483 (Mouse mRNA for PE31/TALLA), M33680 (Human 26-kDa cell surface protein TAPA-1 mRNA, complete cds), T14726 (Human CD53 antigen cDNA), and T23814 (Human gene signature HUMGS05723). The predicted amino add sequence disclosed herein for AR415_{—}4 was searched against the GenPept and GeneSeq amino acid sequence databases using the BLASTX search protocol. The predicted AR415_{—}4 protein demonstrated at least some sequence similarity with sequences identified as D29808 (TALLA-1 [Homo sapiens]), M35252 (tumor-associated antigen [Homo sapiens]), and R22360 (CO-029 tumour associated antigen protein). Based upon sequence similarity, AR415_{—}4 proteins and each homologous protein or peptide may share at least some activity. The TopPredII computer program predicts a potential transmembrane domain within the AR415_{—}4 protein sequence centered around amino acid 100 of SEQ ID NO:35.

Clone “AS63_{—}29”

A polynucleotide of the present invention has been identified as clone “AS63_{—}29”. AS63_{—}29 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AS63_{—}29 is a full-length clone, including the entire coding sequence of a secreted protein (also referred to herein as “AS63_{—}29 protein”).

The nucleotide sequence of the 5′ portion of AS63_{—}29 as presently determined is reported in SEQ ID NO:36. What applicants presently believe is the proper reading frame for the coding region is indicated in SEQ ID NO:37. The predicted amino acid sequence of the AS63_{—}29 protein corresponding to the foregoing nucleotide sequence is reported in SEQ ID NO:37. Amino acids 28 to 40 of SEQ ID NO:37 are a predicted leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the AS63_{—}29 protein. Additional nucleotide sequence from the 3′ portion of AS63_{—}29, including a poly(A) tail, is reported in SEQ ID NO:38.

The EcoRI/NotI restriction fragment obtainable from the deposit containing clone AS63_{—}29 should be approximately 1700 bp. The nucleotide sequence disclosed herein for AS63_{—}29 was searched against the GenBank and GeneSeq nucleotide sequence databases using BLASTN/BLASTX and FASTA search protocols. AS63_{—}29 demonstrated at least some similarity with sequences identified as L26877 (Mus musculus (B20c) heavy chain immunoglobulin variable region gene), T09146 (EST07039 Homo sapiens cDNA clone HIBBP68 5′ end), T23466 (seq3050 Homo sapiens cDNA clone Hy18-Ch13-Charon40-cDNA-100 3′), and W55739 (ma35f05.r1 Life Tech mouse brain Mus musculus cDNA clone 312705 5′). The predicted amino acid sequence disclosed herein for AS63_{—}29 was searched against the GenPept and GeneSeq amino acid sequence databases using the BLASTX search protocol. The predicted AS63_{—}29 protein demonstrated at least some sequence similarity with sequences identified as R04032 (Full length T4 encoded by plasmid pBG381). Based upon sequence similarity, AS_{—}63_{—}29 proteins and each homologous protein or peptide may share at least some activity. The TopPredII computer program predicts a potential transmembrane domain within the AS63_{—}29 protein sequence, near the amino terminus.

Clone “AY304_{—}14”

A polynucleotide of the present invention has been identified as clone “AY304_{—}14”. AY304_{—}14 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AY304_{—}14 is a full-length done, including the entire coding sequence of a secreted protein (also referred to herein as “AY304_{—}14 protein”).

The nucleotide sequence of AY304_{—}14 as presently determined is reported in SEQ ID NO:39, and includes a poly(A) tail. What applicants presently believe to be the proper reading frame and the predicted amino acid sequence of the AY304_{—}14 protein corresponding to the foregoing nucleotide sequence is reported in SEQ ID NO:40.

The EcoRI/NotI restriction fragment obtainable from the deposit containing clone AY304_{—}14 should be approximately 2200 bp.

The nucleotide sequence disclosed herein for AY304_{—}14 was searched against the GenBank and GeneSeq nucleotide sequence databases using BLASTN/BLASTX and FASTA search protocols. AY304_{—}14 demonstrated at least some similarity with sequences identified as AA127688 (zk92f05.s1 Soares pregnant uterus NbHPU Homo sapiens cDNA clone 490305 3′), AA179609 (zp49g11.r1 Stratagene HeLa cell s3 937216 Homo sapiens cDNA clone 612836 5′), AA276253 (vc40f05.r1 Barstead MPLRB1 Mus musculus cDNA clone 777057 5′), H15545 (ym27d04.s1 Homo sapiens cDNA clone 49495 3′ similar to contains PTR5 repetitive element), L08441 (Human autonomously replicating sequence (ARS) mRNA), N34949 (yy49h09.s1 Homo sapiens cDNA clone 276929 3′), R48594 (yj65d07.s1 Homo sapiens cDNA clone 153613 3′), T21160 (Human gene signature HUMGS02466), U43284 (Cloning vector phGFP-S65T, complete sequence, green fluorescent protein (gfp) gene, complete cds), and Z45151 (H. sapiens partial cDNA sequence; clone c-2hh04). The predicted amino acid sequence disclosed herein for AY304_{—}14 was searched against the GenPept and GeneSeq amino acid sequence databases using the BLASTX search protocol. The predicted AY304_{—}14 protein demonstrated at least some sequence similarity with sequences identified as D86984 (similar to yeast adenylate cyclase (S56776) [Homo sapiens]), J01415 (cytochrome oxidase subunit 3 [Homo sapiens]), V00662 (cytochrome oxidase m [Homo sapiens]), and X68948 (envelope glycoprotein [Spleen focus-forming virus]). Based upon sequence similarity, AY304_{—}14 proteins and each homologous protein or peptide may share at least some activity. The TopPredII computer program predicts two potential transmembrane domains within the AY304_{—}14 protein sequence, one centered around amino acid 81 and another around amino acid 120 of SEQ ID NO:40.

Clone “BG160_{—}1”

A polynucleotide of the present invention has been identified as clone “BG160_{—}1”. BG160_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG160_{—}1 is a full-length done, including the entire coding sequence of a secreted protein (also referred to herein as “BG160_{—}1 protein”).

The nucleotide sequence of BG160_{—}1 as presently determined is reported in SEQ ID NO:41, and includes a poly(A) tail. What applicants presently believe to be the proper reading frame and the predicted amino acid sequence of the BG160_{—}1 protein corresponding to the foregoing nucleotide sequence is reported in SEQ ID NO:42. Amino acids 588 to 600 of SEQ ID NO:42 are a predicted leader/signal sequence, with the predicted mature amino acid sequence beginning at amino acid 601. Due to the hydrophobic nature of the predicted leader/signal sequence, it is likely to act as a transmembrane domain should the predicted leader/signal sequence not be separated from the remainder of the BG160_{—}1 protein.

The EcoRI/NotI restriction fragment obtainable from the deposit containing clone BG160_{—}1 should be approximately 2300 bp.

The nucleotide sequence disclosed herein for BG160_{—}1 was searched against the GenBank and GeneSeq nucleotide sequence databases using BLASTN/BLASTX and FASTA search protocols. BG160_{—}1 demonstrated at least some similarity with sequences identified as A60021 (tropomyosin-related protein, neuronal—rat contains element MER27 repetitive element), AA081525 (zn20e02.r1 Stratagene neuroepithelium NT2RAMI 937234 Homo sapiens cDNA clone 547994 5′), AA092565 (115773.seq.F Fetal heart, Lambda ZAP Express Homo sapiens cDNA 5′), D56138 (Human fetal brain cDNA 5′-end GEN-416H11), D61090 (Human fetal brain cDNA 5′-end GEN-155A07), D61184 (Human fetal brain cDNA 5′-end GEN-165A01), L10335 (Homo sapiens neuro-endocrine-specific protein C(NSP) mRNA, complete cds), N21304 (yx53f07.s1 Homo sapiens cDNA clone 265477 3′ similar to SP:A60021 A60021 TROPOMYOSIN-RELATED PROTEIN, NEURONAL), and W95814 (ze07f11.r1 Soares fetal heart NbHH19W Homo sapiens cDNA clone 358317 5′ similar to PIR:A60021). The predicted amino acid sequence disclosed herein for BG160_{—}1 was searched against the GenPept and GeneSeq amino acid sequence databases using the BLASTX search protocol. The predicted BG160_{—}1 protein demonstrated at least some sequence similarity with sequences identified as L10334 (neuroendocrine-specific protein B [Homo sapiens]), L10335 (neuroendocrine-specific protein C [Homo sapiens]). Based upon sequence similarity, BG160_proteins and each homologous protein or peptide may share at least some activity. The TopPredII computer program predicts three potential transmembrane domains within the BG160_{—}1 protein sequence, centered around amino acids 84,484, and 595 of SEQ ID NO:42, respectively.

BG160_{—}1 protein was expressed in a COS cell expression system, and an expressed protein band of approximately 110 kDa was detected in membrane fractions using SDS polyacrylamide gel electrophoresis.

Clone “BO432_{—}4”

A polynucleotide of the present invention has been identified as clone “BO432_{—}4”.

BO432_{—}4 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BO432_{—}4 is a full-length clone, including the entire coding sequence of a secreted protein (also referred to herein as “BO432_{—}4 protein”).

The nucleotide sequence of the 5′ portion of BO432_{—}4 as presently determined is reported in SEQ ID NO:43. An additional internal nucleotide sequence from BO432_{—}4 as presently determined is reported in SEQ ID NO:44. What applicants believe is the proper reading frame and the predicted amino acid sequence encoded by such internal sequence is reported in SEQ ID NO:45. Additional nucleotide sequence from the 3′ portion of BO432_{—}4, including a poly(A) tail, is reported in SEQ ID NO:46.

The EcoRI/NotI restriction fragment obtainable from the deposit containing clone BO432_{—}4 should be approximately 1700 bp.

The nucleotide sequence disclosed herein for BO432_{—}4 was searched against the GenBank and GeneSeq nucleotide sequence databases using BLASTN/BLASTX and FASTA search protocols. BO432_{—}4 demonstrated at least some similarity with sequences identified as AA283626 (zt15e09.s1 Soares NbHTGBC Homo sapiens cDNA clone 713224 3′), AA406486 (zy12g02.r1 Soares NhPu S1 Homo sapiens cDNA clone 753458 5′ similar to WP F35G2.2 CE05809 E. COLI YCAC LIKE), AA570446 (nk62c12.s1 NCI_CGAP_Sch1 Homo sapiens cDNA clone IMAGE:1018102), N55855 (J3389F Homo sapiens cDNA clone J3389 5′), Q10613 (Rianodin receptor gene), T62691 (yc70d10.r1 Homo sapiens cDNA clone 86035 5′), and W90766 (zh79h04.s1 Soares fetal liver spleen 1NFLS S1 Homo sapiens cDNA clone 418327 3′). The predicted amino acid sequence disclosed herein for BO432_{—}4 was searched against the GenPept and GeneSeq amino acid sequence databases using the BLASTX search protocol. The predicted BO432_{—}4 protein demonstrated at least some sequence similarity with sequences identified as Z69637 (F35G2.2 [Caenorhabditis elegans]). Based upon sequence similarity, BO432_{—}4 proteins and each homologous protein or peptide may share at least some activity. The TopPredII computer program predicts a potential transmembrane domain at the amino terminus of the BO432_{—}4 protein sequence. The BO432_{—}4 protein may also contain the bacterial lysR family signature, a motif found in bacterial transcriptional regulators and which is possibly indicative of a helix-turn-helix structure.

Clone “BO538_{—}2”

A polynucleotide of the present invention has been identified as clone “BO538_{—}2”. BO538_{—}2 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BO538_{—}2 is a full-length clone, including the entire coding sequence of a secreted protein (also referred to herein as “BO538_{—}2 protein”).

The nucleotide sequence of the 5′ portion of BO538_{—}2 as presently determined is reported in SEQ ID NO:47. What applicants presently believe is the proper reading frame for the coding region is indicated in SEQ ID NO:48. The predicted amino acid sequence of the BO538_{—}2 protein corresponding to the foregoing nucleotide sequence is reported in SEQ ID NO:48. Additional nucleotide sequence from the 3′ portion of BO538_{—}2, including a poly(A) tail, is reported in SEQ ID NO:49.

The EcoRI/NotI restriction fragment obtainable from the deposit containing clone BO538_{—}2 should be approximately 3000 bp.

The nucleotide sequence disclosed herein for BO538_{—}2 was searched against the GenBank and GeneSeq nucleotide sequence databases using BLASTN/BLASTX and FASTA search protocols. BO538_{—}2 demonstrated at least some similarity with sequences identified as AA503100 (ne44h01.s1 NCI_CGAP_Co3 Homo sapiens cDNA clone 900241), R44035 (yg21g09.s1 Homo sapiens cDNA clone 33167 3′), T21630 (Human gene signature HUMGS03066), and W64854 (me06d12.r1 Soares mouse embryo NbME13.5 14.5 Mus musculus cDNA clone 386711 5′ similar to PIR S40989 S40989 hypothetical protein F55H2.6-Caenorhabditis elegans). The predicted amino acid sequence disclosed herein for BO538_{—}2 was searched against the GenPept and GeneSeq amino acid sequence databases using the BLASTX search protocol. The predicted BO538_{—}2 protein demonstrated at least some sequence similarity with sequences identified as M60525 (nerve growth factor inducible protein [Rattus norvegicus]), R28916 (Type III procollagen), and Z27080 (F55H2.6 [Caenorhabditis elegans]). Based upon sequence similarity, BO538_{—}2 proteins and each homologous protein or peptide may share at least some activity. The TopPredII computer program predicts two potential transmembrane domains within the BO538_{—}2 protein sequence.

Clone “BR595_{—}4”

A polynucleotide of the present invention has been identified as clone “BR595_{—}4”. BR595_{—}4 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BR595_{—}4 is a full-length clone, including the entire coding sequence of a secreted protein (also referred to herein as “BR595_{—}4 protein”).

The nucleotide sequence of the 5′ portion of BR595_{—}4 as presently determined is reported in SEQ ID NO:50. What applicants presently believe is the proper reading frame for the coding region is indicated in SEQ ID NO:51. The predicted amino acid sequence of the BR595_{—}4 protein corresponding to the foregoing nucleotide sequence is reported in SEQ ID NO:51. Additional nucleotide sequence from the 3′ portion of BR595_{—}4, including a poly(A) tail, is reported in SEQ ID NO:52.

The EcoRI/NotI restriction fragment obtainable from the deposit containing clone BR595_{—}4 should be approximately 3000 bp.

The nucleotide sequence disclosed herein for BR595_{—}4 was searched against the GenBank and GeneSeq nucleotide sequence databases using BLASTN/BLASTX and FASTA search protocols. BR595_{—}4 demonstrated at least some similarity with sequences identified as AA443742 (zw95b02.s1 Soares total fetus Nb2HF8 9w Homo sapiens cDNA clone 784683 3′), AA600820 (np45b08.s1 NCI_CGAP_Br1.1 Homo sapiens cDNA clone IMAGE:1129239), T19410 (Human gene signature HUMGS00435), W87465 (zh67c04.s1 Soares fetal liver spleen 1NFLS S1 Homo sapiens cDNA clone 417126 3′), and Z33587 (H. sapiens partial cDNA sequence; clone HEA89P; single read). Based upon sequence similarity, BR595_{—}4 proteins and each homologous protein or peptide may share at least some activity.

Clone “CI490_{—}2”

A polynucleotide of the present invention has been identified as clone “CI490_{—}2”. C1490_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CI490_{—}2 is a full-length clone, including the entire coding sequence of a secreted protein (also referred to herein as “CI490_{—}2 protein”).

The nucleotide sequence of CI490_{—}2 as presently determined is reported in SEQ ID NO:53, and includes a poly(A) tail. What applicants presently believe to be the proper reading frame and the predicted amino acid sequence of the CI490_{—}2 protein corresponding to the foregoing nucleotide sequence is reported in SEQ ID NO:54. Amino acids 64 to 76 of SEQ ID NO:54 are a predicted leader/signal sequence, with the predicted mature amino acid sequence beginning at amino acid 77. Due to the hydrophobic nature of the predicted leader/signal sequence, it is likely to act as a transmembrane domain should the predicted leader/signal sequence not be separated from the remainder of the CI490_{—}2 protein.

The EcoRI/NotI restriction fragment obtainable from the deposit containing clone C1490_{—}2 should be approximately 1200 bp.

The nucleotide sequence disclosed herein for CI490_{—}2 was searched against the GenBank and GeneSeq nucleotide sequence databases using BLASTN/BLASTX and FASTA search protocols. CI490_{—}2 demonstrated at least some similarity with sequences identified as H30751 (yo79a04.r1 Homo sapiens cDNA clone 184110 5′), H49766 (yo24f01.r1 Homo sapiens cDNA clone 178873 5′ similar to SP:S19586 N-METHYL-D-ASPARTATE RECEPTOR GLUTAMATE-BINDING CHAIN), H51158 (yo32d04.r1 Homo sapiens cDNA clone 179623 5′), R85211 (yo41d11.s1 Homo sapiens cDNA clone 180501 3′ similar to SP S19586 N-METHYL-D-ASPARTATE RECEPTOR GLUTAMATE-BINDING CHAIN), S19586 (N-METHYL-D-ASPARTATE RECEPTOR GLUTAMATE-BINDING CHAIN), S61973 (NMDA receptor glutamate-binding subunit [rat, mRNA]), T01031 (Human leucine zipper protein-kinase cDNA sequence), and W56893 (zc01g05.r1 Soares parathyroid tumor NbHPA Homo sapiens cDNA clone 321080 5′ similar to PIR S19586 S19586 N-methyl-D-aspartate receptor glutamate-binding chain—rat). The predicted amino acid sequence disclosed herein for CI490_{—}2 was searched against the GenPept and GeneSeq amino acid sequence databases using the BLASTX search protocol. The predicted C1490_{—}2 protein demonstrated at least some sequence similarity with sequences identified as S61973 (NMDA receptor glutamate-binding subunit [Rattus sp.]) and U08020 (collagen pro-alpha-1 type I chain [Mus musculus]). Based upon sequence similarity, CI490_{—}2 proteins and each homologous protein or peptide may share at least some activity. The TopPredII computer program predicts six potential transmembrane domains within the CI490_{—}2 protein sequence, with the most amino-terminal transmembrane domain centered around amino acid 77 of SEQ ID NO:0.54.

Clone “CI522_{—}1”

A polynucleotide of the present invention has been identified as clone “CI522_{—}1”. CI522_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CI522_{—}1 is a full-length clone, including the entire coding sequence of a secreted protein (also referred to herein as “CI522_{—}1 protein”).

The nucleotide sequence of the 5′ portion of C1522_{—}1 as presently determined is reported in SEQ ID NO:55. What applicants presently believe is the proper reading frame for the coding region is indicated in SEQ ID NO:56. The predicted amino acid sequence of the CI522_{—}1 protein corresponding to the foregoing nucleotide sequence is reported in SEQ ID NO:56. Amino acids 7 to 19 of SEQ ID NO:56 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the C1522_{—}1 protein. Additional nucleotide sequence from the 3′ portion of CI522_{—}1, including a poly(A) tail, is reported in SEQ ID NO:57.

The EcoRI/NotI restriction fragment obtainable from the deposit containing clone CI522_{—}1 should be approximately 1400 bp.

The nucleotide sequence disclosed herein for CI522_{—}1 was searched against the GenBank and GeneSeq nucleotide sequence databases using BLASTN/BLASTX and FASTA search protocols. CI522_{—}1 demonstrated at least some similarity with sequences identified as AA028557 (mi18g05.r1 Soares mouse p3NMF195 Mus musculus cDNA clone 463928 5′), H32238 (EST107136 Rattus sp. cDNA 5′ end), T33525 (EST58140 Homo sapiens cDNA 5′ end similar to None), U66468 (Human cell growth regulator CGR11 mRNA, complete cds), and X00525 (Mouse 28S ribosomal RNA). The predicted amino acid sequence disclosed herein for CI522_{—}1 was searched against the GenPept and GeneSeq amino acid sequence databases using the BLASTX search protocol. The predicted CI522_{—}1 protein demonstrated at least some sequence similarity with sequences identified as U66468 (cell growth regulator CGR11 [Homo sapiens]). Based upon sequence similarity, CI522_{—}1 proteins and each homologous protein or peptide may share at least some activity.

Clone “CN238_{—}1”

A polynucleotide of the present invention has been identified as clone “CN238_{—}1”. CN238_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CN238_{—}1 includes at least a portion of the coding sequence of a secreted protein (also referred to herein as “CN238_{—}1 protein”).

The nucleotide sequence of CN238_{—}1 as presently determined is reported in SEQ ID NO:58, and includes a poly(A) tail. What applicants presently believe to be the proper reading frame and the predicted amino acid sequence of the CN238_{—}1 protein corresponding to the foregoing nucleotide sequence is reported in SEQ ID NO:59.

The EcoRI/NotI restriction fragment obtainable from the deposit containing clone CN238_{—}1 should be approximately 2190 bp.

The nucleotide sequence disclosed herein for CN238_{—}1 was searched against the GenBank and GeneSeq nucleotide sequence databases using BLASTN/BLASTX and FASTA search protocols. CN238_{—}1 demonstrated at least some similarity with sequences identified as AA044097 (zk51b02.r1 Soares pregnant uterus NbHPU Homo sapiens cDNA clone 486315 5′), AA044287 (zk51b02.s1 Soares pregnant uterus NbHPU Homo sapiens cDNA clone 486315 3′), AA045440 (zk67c03.s1 Soares pregnant uterus NbHPU Homo sapiens cDNA clone 487876 3′), AA143007 (zl48f01.r1 Soares pregnant uterus NbHPU Homo sapiens cDNA clone 505177 5′), D51196 (Human fetal brain cDNA 3′-end GEN-016G05), D60310 (Human fetal brain cDNA 3′-end GEN-098AO9), N69344 (yz43e04.s1 Homo sapiens cDNA clone 285822 3′ similar to gb:K00558 TUBULIN ALPHA-1 CHAIN (HUMAN)), W22250 (64B8 Human retina cDNA Tsp509I-cleaved sublibrary Homo), and X01703 (Human gene for alpha-tubulin (b alpha 1)). The predicted amino acid sequence disclosed herein for CN238_{—}1 was searched against the GenPept and GeneSeq amino acid sequence databases using the BLASTX search protocol. The predicted CN238_{—}1 protein demonstrated at least some sequence similarity with sequences identified as K00557 (alpha-tubulin [Homo sapiens]) and U51583 (zinc finger homeodomain enhancer-binding protein-1 [Rattus norvegicus]). Based upon sequence similarity, CN238_{—}1 proteins and each homologous protein or peptide may share at least some activity.

Clone “CO390_{—}1”

A polynucleotide of the present invention has been identified as clone “CO390_{—}1”. CO390_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CO390_{—}1 is a full-length clone, including the entire coding sequence of a secreted protein (also referred to herein as “CO390_{—}1 protein”).

The nucleotide sequence of CO390_{—}1 as presently determined is reported in SEQ ID NO:60, and includes a poly(A) tail. What applicants presently believe to be the proper reading frame and the predicted amino acid sequence of the CO390_{—}1 protein corresponding to the foregoing nucleotide sequence is reported in SEQ ID NO:61.

The EcoRI/NotI restriction fragment obtainable from the deposit containing clone CO390_{—}1 should be approximately 2300 bp. The nucleotide sequence disclosed herein for CO390_{—}1 was searched against the GenBank and GeneSeq nucleotide sequence databases using BLASTN/BLASTX and FASTA search protocols. CO390_{—}1 demonstrated at least some similarity with sequences identified as H84353 (yv85a11.r1 Homo sapiens cDNA clone 249500 5′), L35532 (Pan troglodytes Alu repeat region), N80616 (Genomic clone encoding SAP(Phe)), R53922 (yi03h10.s1 Homo sapiens cDNA clone 138211 3′ similar to contains Alu repetitive element; contains TAR1 repetitive element), X75335 (H. sapiens Alu insertion in COL3A1 gene), X95882 (R. norvegicus mRNA for ATP ligand gated ion channel), and Y09561 (H. sapiens mRNA for P2X7 receptor). The predicted amino acid sequence disclosed herein for CO390_{—}1 was searched against the GenPept and GeneSeq amino acid sequence databases using the BLASTX search protocol. The predicted CO390_{—}1 protein demonstrated at least some sequence similarity with sequences identified as U45448 (P2×1 receptor [Homo sapiens), WO4216 (Rat superior cervical ganglion p2× receptor), X83688 (ATP receptor [Homo sapiens]), X95882 (P2X7 gene product [Rattus norvegicus]), and Y09561 (ATP receptor [Homo sapiens]). Based upon sequence similarity, CO390_{—}1 proteins and each homologous protein or peptide may share at least some activity. The TopPredII computer program predicts a potential transmembrane domain within the CO390_{—}1 protein sequence, centered around amino acid 249 of SEQ ID NO:61. The nucleotide sequence of CO390_{—}1 may contain an Alu repetitive element.

CO390_{—}1 protein was expressed in a COS cell expression system, and an expressed protein band of approximately 75 kDa was detected in conditioned medium using SDS polyacrylamide gel electrophoresis.

Clone “AT20_{—}2”

A polynucleotide of the present invention has been identified as clone “AJ20_{—}2”. AJ20_{—}2 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AJ20_{—}2 is a full-length done, including the entire coding sequence of a secreted protein (also referred to herein as “AJ20_{—}2 protein”).

The nucleotide sequence of the 5′ portion of AJ20_{—}2 as presently determined is reported in SEQ ID NO:62. What applicants presently believe is the proper reading frame for the coding region is indicated in SEQ ID NO:63. The predicted amino acid sequence of the AJ20_{—}2 protein corresponding to the foregoing nucleotide sequence is reported in SEQ ID NO:63. Amino adds 8 to 20 of SEQ ID NO:63 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the AJ20_{—}2 protein. Additional nucleotide sequence from the 3′ portion of AJ20.2, including a poly(A) tail, is reported in SEQ ID NO:64.

The EcoRI/NotI restriction fragment obtainable from the deposit containing clone AJ20_{—}2 should be approximately 850 bp.

The nucleotide sequence disclosed herein for AJ20_{—}2 was searched against the GenBank and GeneSeq nucleotide sequence databases using BLASTN/BLASTX and FASTA search protocols. No hits were found in the database.

Clone “AR440_{—}1”

A polynucleotide of the present invention has been identified as clone “AR440_{—}1”. AR440_{—}1 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AR440_{—}1 is a full-length clone, including the entire coding sequence of a secreted protein (also referred to herein as “AR440_{—}1 protein”).

The partial nucleotide sequence of AR440_{—}1, including its 3′ end and a poly(A) tail, as presently determined is reported in SEQ ID NO:66. What applicants presently believe is the proper reading frame for the coding region is indicated in SEQ ID NO:67. The predicted amino acid sequence of the AR440_{—}1 protein corresponding to the foregoing nucleotide sequence is reported in SEQ ID NO:67. Additional nucleotide sequence from the 5′ portion of AR440_{—}1 is reported in SEQ ID NO:65.

The EcoRI/NotI restriction fragment obtainable from the deposit containing clone AR440_{—}1 should be approximately 1400 bp.

The nucleotide sequence disclosed herein for AR440_{—}1 was searched against the GenBank and GeneSeq nucleotide sequence databases using BLASTN/BLASTX and FASTA search protocols. No hits were found in the database. The nucleotide sequence of AR440_{—}1 indicates that it may contain an Alu repetitive element.

Clone “AS164_{—}1”

A polynucleotide of the present invention has been identified as clone “AS164_{—}1”. AS164_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AS164_{—}1 is a full-length done, including the entire coding sequence of a secreted protein (also referred to herein as “AS164_{—}1 protein”). The nucleotide sequence of AS164_{—}1 as presently determined is reported in SEQ ID NO:68, and includes a poly(A) tail. What applicants presently believe to be the proper reading frame and the predicted amino acid sequence of the AS164_{—}1 protein corresponding to the foregoing nucleotide sequence is reported in SEQ ID NO:69.

The EcoRI/NotI restriction fragment obtainable from the deposit containing clone AS164_{—}1 should be approximately 1600 bp.

The nucleotide sequence disclosed herein for AS164_{—}1 was searched against the GenBank and GeneSeq nucleotide sequence databases using BLASTN/BLASTX and FASTA search protocols. AS164_{—}1 demonstrated at least some similarity with sequences identified as H24668 (yl40h10.r1 Homo sapiens cDNA clone 160771 5′), N29757 (yw90h10.s1 Homo sapiens cDNA clone 259555 3′), T62184 (yb96d08.r1 Homo sapiens cDNA clone 79023 5′), Z69706 (Human DNA sequence from cosmid COS12 from a contig from the tip of the short arm of chromosome 16, spanning 2 Mb of 16p13.3. Contains ESTs, Flanking sequences of 3′ alpha globin H), and Z69890 (Human DNA sequence from cosmid RJ14 from a contig from the tip of the short arm of chromosome 16, spanning 2 Mb of 16p13.3. Contains ESTs and CpG island). The predicted amino acid sequence disclosed herein for AS64_{—}1 was searched against the GenPept and GeneSeq amino acid sequence databases using the BLASTX search protocol. The predicted AS164_{—}1 protein demonstrated at least some similarity to sequences identified as A20359_{—}1 (ryanodine receptor gene product [Homo sapiens]) and U78866 (putative arginine-aspartate-rich RNA binding protein [Arabidopsis thaliana]). Based upon sequence similarity, AS164_{—}1 proteins and each similar protein or peptide may share at least some activity. The predicted AS164_{—}1 protein sequence also contains repeated Asp-Arg RNA-binding motifs.

Clone “AX8_{—}1”

A polynucleotide of the present invention has been identified as clone “AX8_{—}1”. AX8_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AX8_{—}1 is a full-length clone, including the entire coding sequence of a secreted protein (also referred to herein as “AX8_{—}1 protein”).

The nucleotide sequence of AX8_{—}1 as presently determined is reported in SEQ ID NO:70, and includes a poly(A) tail. What applicants presently believe to be the proper reading frame and the predicted amino acid sequence of the AX8_{—}1 protein corresponding to the foregoing nucleotide sequence is reported in SEQ ID NO:71. Amino acids 106 to 118 of SEQ ID NO:71 are a predicted leader/signal sequence, with the predicted mature amino acid sequence beginning at amino acid 119. Due to the hydrophobic nature of the predicted leader/signal sequence, it is likely to act as a transmembrane domain should the predicted leader/signal sequence not be separated from the remainder of the AX8_{—}1 protein.

The EcoRI/NotI restriction fragment obtainable from the deposit containing clone AX8_{—}1 should be approximately 2300 bp.

The nucleotide sequence disclosed herein for AX8_{—}1 was searched against the GenBank and GeneSeq nucleotide sequence databases using BLASTN/BLASTX and FASTA search protocols. No hits were found in the database. The TopPredII computer program predicts three potential transmembrane domains within the AX8_{—}1 protein sequence, centered around amino acids 111, 144, and 182 of SEQ ID NO:71, respectively.

AX8_{—}1 protein was expressed in a COS cell expression system, and an expressed protein band of approximately 35 kDa was detected in conditioned medium and membrane fractions using SDS polyacrylamide gel electrophoresis.

Clone “BD176_{—}3”

A polynucleotide of the present invention has been identified as clone “BD176_{—}3”. BD176_{—}3 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD176_{—}3 is a full-length clone, including the entire coding sequence of a secreted protein (also referred to herein as “BD176_{—}3 protein”).

The nucleotide sequence of the 5′ portion of BD1763 as presently determined is reported in SEQ ID NO:72. What applicants presently believe is the proper reading frame for the coding region is indicated in SEQ ID NO:73. The predicted amino acid sequence of the BD176_{—}3 protein corresponding to the foregoing nucleotide sequence is reported in SEQ ID NO:73. Amino acids 2 to 14 of SEQ ID NO:73 are a predicted leader/signal sequence, with the predicted mature amino acid sequence beginning at amino acid 15. Due to the hydrophobic nature of the predicted leader/signal sequence, it is likely to act as a transmembrane domain should the predicted leader/signal sequence not be separated from the remainder of the BD176_{—}3 protein. Additional nucleotide sequence from the 3′ portion of BD176_{—}3, including a poly(A) tail, is reported in SEQ ID NO:74.

The EcoRI/NotI restriction fragment obtainable from the deposit containing clone BD176_{—}3 should be approximately 1300 bp.

The nucleotide sequence disclosed herein for BD176_{—}3 was searched against the GenBank and GeneSeq nucleotide sequence databases using BLASTN/BLASTX and FASTA search protocols. BD176_{—}3 demonstrated at least some similarity with sequences identified as AA029679 (ze94g10.r1 Soares fetal heart NbHH19W Homo sapiens cDNA clone 366690 5′), D45913 (Mouse NLRR-1 mRNA for leucine-rich-repeat protein, complete cds), R55610 (yg88h08.r1 Homo sapiens cDNA clone 40606 5′), and T07640 (EST05530 Homo sapiens cDNA clone HFBEM16). The predicted amino acid sequence disclosed herein for BD176_{—}3 was searched against the GenPept and GeneSeq amino acid sequence databases using the BLASTX search protocol. The predicted BD176_{—}3 protein demonstrated at least some similarity to sequences identified as D45913 (leucine-rich-repeat protein [Mus musculus]) and M59472 (asparagine-rich antigen Pfa55-6 [Plasmodium falciparum]). Based upon sequence similarity, BD176_{—}3 proteins and each similar protein or peptide may share at least some activity.

Clone “BD339_{—}1”

A polynucleotide of the present invention has been identified as clone “BD339_{—}1”. BD339_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD339_{—}1 is a full-length clone, including the entire coding sequence of a secreted protein (also referred to herein as “BD339_{—}1 protein”).

The nucleotide sequence of BD339_{—}1 as presently determined is reported in SEQ ID NO:75, and includes a poly(A) tail. What applicants presently believe to be the proper reading frame and the predicted amino acid sequence of the BD339_{—}1 protein corresponding to the foregoing nucleotide sequence is reported in SEQ ID NO:76.

The EcoRI/NotI restriction fragment obtainable from the deposit containing clone BD339_{—}1 should be approximately 650 bp.

The nucleotide sequence disclosed herein for BD339_{—}1 was searched against the GenBank and GeneSeq nucleotide sequence databases using BLASTN/BLASTX and FASTA search protocols. BD339_{—}1 demonstrated at least some similarity with sequences identified as H82422 (yu80d08.s1 Homo sapiens cDNA clone 240111 3), N62058 (EST53c05 Homo sapiens cDNA clone), U21730 Human 5′-nucleotidase (CD73)), WO1979 (za30h09.r1 Soares fetal liver spleen 1NFLS Homo sapiens cDNA clone 294113 5′), and WO2015 (za32b11.r1 Soares fetal liver spleen 1NFLS Homo sapiens cDNA clone 294237 5′). Based upon sequence similarity, BD39_{—}1 proteins and each similar protein or peptide may share at least some activity. The TopPredII computer program predicts three potential transmembrane domains within the BD339_{—}1 protein sequence, centered around amino acids 14,46, and 76 of SEQ ID NO:76, respectively.

Clone “BD427_{—}1”

A polynucleotide of the present invention has been identified as clone “BD427_{—}1”. BD427_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD427_{—}1 is a full-length clone, including the entire coding sequence of a secreted protein (also referred to herein as “BD427_protein”).

The nucleotide sequence of BD427_{—}1 as presently determined is reported in SEQ ID NO:77, and includes a poly(A) tail. What applicants presently believe to be the proper reading frame and the predicted amino acid sequence of the BD427_{—}1 protein corresponding to the foregoing nucleotide sequence is reported in SEQ ID NO:78.

The EcoRI/NotI restriction fragment obtainable from the deposit containing clone BD427_{—}1 should be approximately 1810 bp.

The nucleotide sequence disclosed herein for BD427_{—}1 was searched against the GenBank and GeneSeq nucleotide sequence databases using BLASTN/BLASTX and FASTA search protocols. BD427_{—}1 demonstrated at least some similarity with sequences identified as BD427_{—}1 demonstrated at least some similarity with sequences identified as AA027122 (zk04a03.r1 Soares pregnant uterus NbHPU Homo sapiens cDNA clone 469516 5′), N24735 (yx56b02.s1 Homo sapiens cDNA clone 265707 3′), and W84644 (zd91a06.r1 Soares fetal heart NbHH19W Homo sapiens cDNA clone 356818 5′). Based upon sequence similarity, BD427_{—}1 proteins and each similar protein or peptide may share at least some activity.

Clone “BL229_{—}22”

A polynucleotide of the present invention has been identified as clone “BL229_{—}22”. BL229_{—}22 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL29_{—}22 is a full-length done, including the entire coding sequence of a secreted protein (also referred to herein as “BL229_{—}22 protein”).

The nucleotide sequence of the 5′ portion of BL229_{—}22 as presently determined is reported in SEQ ID NO:79. What applicants presently believe is the proper reading frame for the coding region is indicated in SEQ ID NO:80. The predicted amino acid sequence of the BL229_{—}22 protein corresponding to the foregoing nucleotide sequence is reported in SEQ ID NO:80. Additional nucleotide sequence from the 3′ portion of BL229-22, including a poly(A) tail, is reported in SEQ ID NO:81.

The EcoRI/NotI restriction fragment obtainable from the deposit containing clone BL229_{—}22 should be approximately 870 bp.

The nucleotide sequence disclosed herein for BL229_{—}22 was searched against the GenBank and GeneSeq nucleotide sequence databases using BLASTN/BLASTX and FASTA search protocols. No hits were found in the database.

Clone “BV123_{—}16”

A polynucleotide of the present invention has been identified as clone “BV123_{—}16”. BV123_{—}16 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV123_{—}16 is a full-length clone, including the entire coding sequence of a secreted protein (also referred to herein as “BV123_{—}16 protein”).

The nucleotide sequence of BV123_{—}16 as presently determined is reported in SEQ ID NO:82, and includes a poly(A) tail. What applicants presently believe to be the proper reading frame and the predicted amino acid sequence of the BV123_{—}16 protein corresponding to the foregoing nucleotide sequence is reported in SEQ ID NO:83.

The EcoRI/NotI restriction fragment obtainable from the deposit containing clone BV123_{—}16 should be approximately 1080 bp.

The nucleotide sequence disclosed herein for BV123_{—}16 was searched against the GenBank and GeneSeq nucleotide sequence databases using BLASTN/BLASTX and FASTA search protocols. BV123_{—}16 demonstrated at least some similarity with sequences identified as H29610 (ym61e03.s1 Homo sapiens cDNA clone 52653 3′), H52374 (yq81b12.r1 Homo sapiens cDNA clone 202175 5′), H66213 (yu16h10.s1 Homo sapiens cDNA), L08092 (Homo sapiens dystrophin (DMD) gene, intron 7, transposon-like sequence), L35670 (Homo sapiens (subclone H810g5 from P1 35H5C8) DNA sequence), M62716 (Human CSP-B gene flanking sequence), N46985 (yy83a05.s1 Homo sapiens cDNA clone 280112 3′), R94603 (yq38a04.s1 Homo sapiens cDNA clone 198030 3′), U91321 (Human chromosome 16p13 BAC clone CIT987SK-363E6, complete sequence), and Z82200 (Human DNA sequence from clone J333E231). Based upon sequence similarity, BV123_{—}16 proteins and each similar protein or peptide may share at least some activity.

Clone “CH377_{—}1”

A polynucleotide of the present invention has been identified as clone “CH377_{—}1”. CH377_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CH377_{—}1 is a full-length done, including the entire coding sequence of a secreted protein (also referred to herein as “CH377_{—}1 protein”).

The nucleotide sequence of CH377_{—}1 as presently determined is reported in SEQ ID NO:84, and includes a poly(A) tail. What applicants presently believe to be the proper reading frame and the predicted amino acid sequence of the CH377_{—}1 protein corresponding to the foregoing nucleotide sequence is reported in SEQ ID NO:85. Amino acids 5 to 17 of SEQ ID NO:85 are a predicted leader/signal sequence, with the predicted mature amino acid sequence beginning at amino acid 18. Due to the hydrophobic nature of the predicted leader/signal sequence, it is likely to act as a transmembrane domain should the predicted leader/signal sequence not be separated from the remainder of the CH377_{—}1 protein.

The EcoRI/NotI restriction fragment obtainable from the deposit containing clone CH377_{—}1 should be approximately 570 bp. The nucleotide sequence disclosed herein for CH377_{—}1 was searched against the GenBank and GeneSeq nucleotide sequence databases using BLASTN/BLASTX and FASTA search protocols. CH377_{—}1 demonstrated at least some similarity with sequences identified as AA507382 (nh73b01.s1 NCI_CGAP_Br1.1 Homo sapiens cDNA clone IMAGE 964105) and N70479 (za74f12.s1 Homo sapiens cDNA clone 298319 3′). Based upon sequence similarity, CH377_{—}1 proteins and each similar protein or peptide may share at least some activity.

Clone “BD441_{—}1”

A polynucleotide of the present invention has been identified as clone “BD441_{—}1”. BD441_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD441_{—}1 is a full-length clone, including the entire coding sequence of a secreted protein (also referred to herein as “BD441_{—}1 protein”).

The nucleotide sequence of the 5′ portion of BD441_{—}1 as presently determined is reported in SEQ ID NO:86. An additional internal nucleotide sequence from BD441_{—}1 as presently determined is reported in SEQ ID NO:87. What applicants believe is the proper reading frame and the predicted amino acid sequence encoded by such internal sequence is reported in SEQ ID NO:88. Additional nucleotide sequence from the 3′ portion of BD441_{—}1, including a poly(A) tail, is reported in SEQ ID NO:89.

The EcoRI/NotI restriction fragment obtainable from the deposit containing clone BD441_{—}1 should be approximately 2400 bp.

The predicted amino acid sequence disclosed herein for BD441_{—}1 was searched against the GenPept and GeneSeq amino acid sequence databases using the BLASTX search protocol. The predicted BD441_{—}1 protein demonstrated at least some similarity to sequences identified as X61615 (leukemia inhibitory factor receptor [Homo sapiens]). Based upon sequence similarity, BD441_{—}1 proteins and each similar protein or peptide may share at least some activity.

Clone “BD441_{—}2”

A polynucleotide of the present invention has been identified as clone “BD441_{—}2”. BD441_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD441_{—}2 is a full-length clone, including the entire coding sequence of a secreted protein (also referred to herein as “BD441_{—}2 protein”).

The nucleotide sequence of the 5′ portion of BD441_{—}2 as presently determined is reported in SEQ ID NO:90. What applicants presently believe is the proper reading frame for the coding region is indicated in SEQ ID NO:91. The predicted amino acid sequence of the BD441_{—}2 protein corresponding to the foregoing nucleotide sequence is reported in SEQ ID NO:91. Additional nucleotide sequence from the 3′ portion of BD441_{—}2, including a poly(A) tail, is reported in SEQ ID NO:92.

The EcoRI/NotI restriction fragment obtainable from the deposit containing clone BD441_{—}2 should be approximately 1200 bp.

The predicted amino acid sequence disclosed herein for BD441_{—}2 was searched against the GenPept and GeneSeq amino acid sequence databases using the BLASTX search protocol. The predicted BD441_{—}2 protein demonstrated at least some similarity to sequences identified as X61615 (leukemia inhibitory factor receptor [Homo sapiens]). Based upon sequence similarity, BD441_{—}2 proteins and each similar protein or peptide may share at least some activity.

Clone “BG102_{—}3”

A polynucleotide of the present invention has been identified as clone “BG102_{—}3”. BG102_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG102_{—}3 is a full-length done, including the entire coding sequence of a secreted protein (also referred to herein as “BG102_{—}3 protein”).

The nucleotide sequence of BG102_{—}3 as presently determined is reported in SEQ ID NO:93, and includes a poly(A) tail. What applicants presently believe to be the proper reading frame and the predicted amino acid sequence of the BG102_{—}3 protein corresponding to the foregoing nucleotide sequence is reported in SEQ ID NO:94. Amino acids 11 to 23 of SEQ ID NO:94 are a predicted leader/signal sequence, with the predicted mature amino acid sequence beginning at amino acid 24. Due to the hydrophobic nature of the predicted leader/signal sequence, it is likely to act as a transmembrane domain should the predicted leader/signal sequence not be separated from the remainder of the BG102_{—}3 protein.

The EcoRI/NotI restriction fragment obtainable from the deposit containing clone BG102_{—}3 should be approximately 1100 bp.

The nucleotide sequence disclosed herein for BG102_{—}3 was searched against the GenBank and GeneSeq nucleotide sequence databases using BLASTN/BLASTX and FASTA search protocols. BG102_{—}3 demonstrated at least some similarity with sequences identified as AC002078 (Human BAC clone RG111H14 from 7q22, complete sequence), L11910 (Human retinoblastoma susceptibility gene exons 1-27, complete cds), U62317 (Chromosome 22q13 BAC Clone CIT987SK-384D8 complete sequence), Z54147 (Human DNA sequence from cosmid L129H7, Huntington's Disease Region, chromosome 4p16.3 contains CpG island), Z75747 (Human DNA sequence from cosmid U96H1, between markers DXS366 and DXS87 on chromosome X*), and Z80899 (Human DNA sequence from cosmid F1121 on chromosome 6). The predicted amino acid sequence disclosed herein for BG102_{—}3 was searched against the GenPept and GeneSeq amino acid sequence databases using the BLASTX search protocol. The predicted BG102_{—}3 protein demonstrated at least some similarity to sequences identified as M13100 (unknown protein [Rattus norvegicus]) and U15647 (reverse transcriptase [Mus musculus]). Based upon sequence similarity, BG102_{—}3 proteins and each similar protein or peptide may share at least some activity. The nucleotide sequence of BG102_{—}3 indicates that it may contain an L1 repetitive element.

BG102_{—}3 protein was expressed in a COS cell expression system, and an expressed protein band of approximately 55 kDa was detected in conditioned medium using SDS polyacrylamide gel electrophoresis.

Clone “BK158_{—}1”

A polynucleotide of the present invention has been identified as clone “BK158_{—}1”. BK158_{—}1 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BK158_{—}1 is a full-length clone, including the entire coding sequence of a secreted protein (also referred to herein as “BK158_{—}1 protein”).

The nucleotide sequence of BK158_{—}1 as presently determined is reported in SEQ ID NO:95, and includes a poly(A) tail. What applicants presently believe to be the proper reading frame and the predicted amino acid sequence of the BK158_{—}1 protein corresponding to the foregoing nucleotide sequence is reported in SEQ ID NO:96.

The EcoRI/NotI restriction fragment obtainable from the deposit containing clone BK158_{—}1 should be approximately 1150 bp.

The nucleotide sequence disclosed herein for BK158_{—}1 was searched against the GenBank and GeneSeq nucleotide sequence databases using BLASTN/BLASTX and FASTA search protocols. BK158_{—}1 demonstrated at least some similarity with sequences identified as N39195 (yv26e08.s1 Homo sapiens cDNA clone 243878 3′) and N45263 (yv26e08.r1 Homo sapiens cDNA clone 243878 5′). Based upon sequence similarity, BK158_{—}1 proteins and each similar protein or peptide may share at least some activity.

BK158_{—}1 protein was expressed in a COS cell expression system, and an expressed protein band of approximately 28 kDa was detected in conditioned medium and membrane fractions using SDS polyacrylamide gel electrophoresis.

Clone “BP163_{—}1”

A polynucleotide of the present invention has been identified as clone “BP163_{—}1”. BP163_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BP163_{—}1 is a fill-length clone, including the entire coding sequence of a secreted protein (also referred to herein as “BP163_{—}1 protein”).

The nucleotide sequence of the 5′ portion of BP163_{—}1 as presently determined is reported in SEQ ID NO:97. What applicants presently believe is the proper reading frame for the coding region is indicated in SEQ ID NO:98. The predicted amino acid sequence of the BP163_{—}1 protein corresponding to the foregoing nucleotide sequence is reported in SEQ ID NO:98. Additional nucleotide sequence from the 3′ portion of BP163_{—}1, including a poly(A) tail, is reported in SEQ ID NO:99.

The EcoRI/NotI restriction fragment obtainable from the deposit containing clone BP163_{—}1 should be approximately 1240 bp.

The nucleotide sequence disclosed herein for BP163_{—}1 was searched against the GenBank and GeneSeq nucleotide sequence databases using BLASTN/BLASTX and FASTA search protocols. BP163_{—}1.demonstrated at least some similarity with sequences identified as AA187086 (zp58h06.r1 Stratagene endothelial cell 937223 Homo sapiens cDNA clone 624443 5′ similar to IR G285943 G285943 ORF, COMPLETE CDS), AA301506 (EST14475 Testis tumor Homo sapiens cDNA 5′ end similar to hypothetical protein (GB D14659)), D14659 (Human mRNA for KIAA0103 gene, complete cds), and W57328 (ma26d10.r1 Life Tech mouse brain Mus musculus cDNA clone). The predicted amino acid sequence disclosed herein for BP163_{—}1 was searched against the GenPept and GeneSeq amino acid sequence databases using the BLASTX search protocol. The predicted BP163_{—}1 protein demonstrated at least some similarity to sequences identified as D14659 (KIAA0103 [Homo sapiens]). Based upon sequence similarity, BP163_{—}1 proteins and each similar protein or peptide may share at least some activity.

Clone “BZ16_{—}3”

A polynucleotide of the present invention has been identified as clone “BZ16_{—}3”. BZ16_{—}3 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BZ16_{—}3 is a full-length clone, including the entire coding sequence of a secreted protein (also referred to herein as “BZ16_{—}3 protein”).

The partial nucleotide sequence of BZ16_{—}3, including its 3′ end and a poly(A) tail, as presently determined is reported in SEQ ID NO:101. What applicants presently believe is the proper reading frame for the coding region is indicated in SEQ ID NO:102. The predicted amino acid sequence of the BZ16_{—}3 protein corresponding to the foregoing nucleotide sequence is reported in SEQ ID NO:102. Additional nucleotide sequence from the 5′ portion of BZ16_{—}3 is reported in SEQ ID NO:100.

The EcoRI/NotI restriction fragment obtainable from the deposit containing clone BZ16_{—}3 should be approximately 2120 bp.

The nucleotide sequence disclosed herein for BZ16_{—}3 was searched against the GenBank and GeneSeq nucleotide sequence databases using BLASTN/BLASTX and FASTA search protocols. BZ16_{—}3 demonstrated at least some similarity with sequences identified as F06886 (H. sapiens partial cDNA sequence; clone c-1nf02), F06870 (H. sapiens partial cDNA sequence; clone c-1nc11), N53511 (yz26b08.s1 Homo sapiens cDNA clone 284151 3′), T65313 (yc79g12.s1 Homo sapiens cDNA clone 22132 3′), U00084 (Haemophilus influenzae), W44815 (zc21d01.s1 Soares senescent fibroblasts NbHSF Homo sapiens cDNA clone 322945 3′), and Z49128 (Caenorhabditis elegans cosmid M03C11). The predicted amino acid sequence disclosed herein for BZ16_{—}3 was searched against the GenPept and GeneSeq amino acid sequence databases using the BLASTX search protocol. The predicted BZ16_{—}3 protein demonstrated at least some similarity to sequences identified as D26185 (cell division protein [Bacillus subtilis]), L46096 (HEAHI465_{—}1 cell division protein [Haemophilus influenzae]), and Z49128 (CEM03C11_{—}5 M03C115 [Caenorhabditis elegans]). The BZ16_{—}3 protein demonstrated at least some similarity to ATP-dependent proteases such as ftsH. Based upon sequence similarity, BZ16_{—}3 proteins and each similar protein or peptide may share at least some activity.

Clone “CC182_{—}1”

A polynucleotide of the present invention has been identified as clone “CC182_{—}1”. CC182_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC182_{—}1 is a full-length clone, including the entire coding sequence of a secreted protein (also referred to herein as “CC182_{—}1 protein”).

The nucleotide sequence of CC182_{—}1 as presently determined is reported in SEQ ID NO:103, and includes a poly(A) tail. What applicants presently believe to be the proper reading frame and the predicted amino acid sequence of the CC182_{—}1 protein corresponding to the foregoing nucleotide sequence is reported in SEQ ID NO:104. Amino acids 26 to 38 of SEQ ID NO:104 are a predicted leader/signal sequence, with the predicted mature amino acid sequence beginning at amino acid 39. Due to the hydrophobic nature of the predicted leader/signal sequence, it is likely to act as a transmembrane domain should the predicted leader/signal sequence not be separated from the remainder of the CC182_]protein.

The EcoRI/NotI restriction fragment obtainable from the deposit containing clone CC182_{—}1 should be approximately 1600 bp.

The nucleotide sequence disclosed herein for CC182_{—}1 was searched against the GenBank and GeneSeq nucleotide sequence databases using BLASTN/BLASTX and FASTA search protocols. CC182_{—}1 demonstrated at least some similarity with sequences identified as H61159 (yu37f08.s1 Homo sapiens cDNA clone 236007 3′ similar to contains L1 repetitive element), L09709 (Human lysosomal-associated membrane glycoprotein-2 (LAMP2) gene, 5′ end of CDS and flanking region), W44797 (zb98e10.s1 Soares parathyroid tumor NbHPA Homo sapiens cDNA clone 320874 3′ similar to contains Alu repetitive element), and X62167 (H. sapiens mRNA for P2 protein of peripheral myelin). Based upon sequence similarity,. CC182_{—}1 proteins and each similar protein or peptide may share at least some activity. The nucleotide sequence of CC182_{—}1 indicates that it may contain an L1 repetitive element and/or a MER42C repetitive element.

Clone “CG109_{—}1”

A polynucleotide of the present invention has been identified as clone “CG109_{—}1”. CG109_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CG109_{—}1 is a full-length done, including the entire coding sequence of a secreted protein (also referred to herein as “CG109_{—}1 protein”).

The nucleotide sequence of CG109_{—}1 as presently determined is reported in SEQ ID NO:105, and includes a poly(A) tail. What applicants presently believe to be the proper reading frame and the predicted amino acid sequence of the CG109_{—}1 protein corresponding to the foregoing nucleotide sequence is reported in SEQ ID NO:106.

The EcoRI/NotI restriction fragment obtainable from the deposit containing clone CG109_{—}1 should be approximately 600 bp.

The nucleotide sequence disclosed herein for CG109_{—}1 was searched against the GenBank and GeneSeq nucleotide sequence databases using BLASTN/BLASTX and FASTA search protocols. No hits were found in the database.

Clone “CT397_{—}1”

A polynucleotide of the present invention has been identified as clone “CJ397_{—}1”. CJ397_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CJ397_{—}1 is a full-length clone, including the entire coding sequence of a secreted protein (also referred to herein as “CJ397_{—}1 protein”).

The nucleotide sequence of the 5′ portion of CJ397_{—}1 as presently determined is reported in SEQ ID NO:107. An additional internal nucleotide sequence from CJ397_{—}1 as presently determined is reported in SEQ ID NO:108. What applicants believe is the proper reading frame and the predicted amino acid sequence encoded by such internal sequence is reported in SEQ ID NO:109. Additional nucleotide sequence from the 3′ portion of CJ397_{—}1, including a poly(A) tail, is reported in SEQ ID NO:110.

The EcoRI/NotI restriction fragment obtainable from the deposit containing clone CJ397_{—}1 should be approximately 1900 bp.

The nucleotide sequence disclosed herein for CJ397_{—}1 was searched against the GenBank and GeneSeq nucleotide sequence databases using BLASTN/BLASTX and FASTA search protocols. CJ397_{—}1 demonstrated at least some similarity with sequences identified as H18685 (yn52b08.s1 Homo sapiens cDNA clone 172023 3′), H46001 (yo13f06.s1 Homo sapiens cDNA clone 177827 3′), and T77612 (yc91f06.r1 Homo sapiens cDNA clone 23298 5′). Based upon sequence similarity, CJ397_{—}1 proteins and each similar protein or peptide may share at least some activity.

Clone “AM795_{—}4”

A polynucleotide of the present invention has been identified as clone “AM795_{—}4”. AM795_{—}4 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AM795_{—}4 is a full-length clone, including the entire coding sequence of a secreted protein (also referred to herein as “AM795_{—}4 protein”).

The nucleotide sequence of AM795_{—}4 as presently determined is reported in SEQ ID NO:111, and includes a poly(A) tail. What applicants presently believe to be the proper reading frame and the predicted amino acid sequence of the AM795_{—}4 protein corresponding to the foregoing nucleotide sequence is reported in SEQ ID NO:112. Amino acids 9 to 21 of SEQ ID NO:112 are a predicted leader/signal sequence, with the predicted mature amino acid sequence beginning at amino acid 22. Amino acids 138 to 150 of SEQ ID NO:112 are a possible leader/signal sequence, with the predicted mature amino acid sequence beginning at amino acid 151. Due to the hydrophobic nature of the predicted and the possible leader/signal sequences, each of such sequences may act as a transmembrane domain should that leader/signal sequence not be separated from the remainder of the AM795_{—}4 protein.

The EcoRI/NotI restriction fragment obtainable from the deposit containing clone AM795_{—}4 should be approximately 1900 bp.

The nucleotide sequence disclosed herein for AM795_{—}4 was searched against the GenBank and GeneSeq nucleotide sequence databases using BLASTN/BLASTX and FASTA search protocols. AM795_{—}4 demonstrated at least some similarity with sequences identified as AF002700 (Homo sapiens GDNF family receptor alpha 2 (GFRalpha2) mRNA, complete cds), H05619 (yl70a10.s1 Homo sapiens cDNA clone 43207 3′), U46493 (Cloning vector pFlp recombinase gene, complete cds), U59486 (Rattus norvegicus GDNF receptor alpha mRNA, complete cds), V00248 (Human Ret ligand retL2 cDNA), W73633 (zd55h01.s1 Soares fetal heart NbHH19W Homo sapiens cDNA clone 344593 3′, mRNA sequence), and W73681 (zd55h01.r1 Soares fetal heart NbHH19W Homo sapiens cDNA clone 344593 5′, mRNA sequence). The predicted amino acid sequence disclosed herein for AM795_{—}4 was searched against the GenPept and GeneSeq amino acid sequence databases using the BLASTX search protocol. The predicted AM795_{—}4 protein demonstrated at least some similarity to sequences identified as AF002700 (GDNF family receptor alpha 2 (GFRalpha2) [Homo sapiens]), U59486 (GDNF receptor alpha [Rattus norvegicus]), and W37460 (Human Ret ligand retL2 cDNA). A receptor complex comprised of TmR1 (GDNFR alpha) and Ret was found to be capable of mediating both GDNF and NTN signaling. The receptor called TmR2, identified based on homology to TmR1, is 48% identical to TmR1 and is encoded by a gene located on the short arm of chromosome 8. TmR2 is attached to the cell surface via a GPI-linkage, and can mediate both NTN and GDNF signaling through Ret in vitro (Baloh et al., 1997, Neuron 18(5): 793-802, which is incorporated by reference herein). Based upon sequence similarity, AM795_{—}4 proteins and each similar protein or peptide may share at least some activity.

AM795_{—}4 protein was expressed in a COS cell expression system, and an expressed protein band of approximately 76 kDa was detected in conditioned media fractions using SDS polyacrylamide gel electrophoresis.

Clone “AT340_{—}1”

A polynucleotide of the present invention has been identified as clone “AT340_{—}1”. AT340_{—}1 was isolated from a human adult blood (lymphocytes and dendritic cells treated with mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AT340_{—}1 is a full-length done, including the entire coding sequence of a secreted protein (also referred to herein as “AT340_{—}1 protein”).

The partial nucleotide sequence of AT340_{—}1, including its 3′ end and a poly(A) tail, as presently determined is reported in SEQ ID NO:114. What applicants presently believe is the proper reading frame for the coding region is indicated in SEQ ID NO:115. The predicted amino acid sequence of the AT340_{—}1 protein corresponding to the foregoing nucleotide sequence is reported in SEQ ID NO:115. Amino acids 12 to 24 of SEQ ID NO:115 are a predicted leader/signal sequence, with the predicted mature amino acid sequence be ginning at amino acid 25. Due to the hydrophobic nature of the predicted leader/signal sequence, it is likely to act as a transmembrane domain should the predicted leader/signal sequence not be separated from the remainder of the AT340_{—}1 protein.

Additional nucleotide sequence from the 5′ portion of AT340_{—}1 is reported in SEQ ID NO:113.

The EcoRI/NotI restriction fragment obtainable from the deposit containing clone AT340_{—}1 should be approximately 1100 bp.

The nucleotide sequence disclosed herein for AT340_{—}1 was searched against the GenBank and GeneSeq nucleotide sequence databases using BLASTN/BLASTX and FASTA search protocols. AT340_{—}1 demonstrated at least some similarity with sequences identified as AA039343 (zk39g04.s1 Soares pregnant uterus NbHPU Homo sapiens cDNA clone 485238 3′), R68951 (yi43g06.r1 Homo sapiens cDNA clone 142042 5′ similar to SP:C35D10.1 CE01190), R77532 (yi76c01.r1 Homo sapiens cDNA), R92619 (yq04a04.r1 Homo sapiens cDNA clone 195918 5′ similar to SP:C35D10.1 CE01190), and W60997 (zc99f09s1 Pancreatic Islet Homo sapiens cDNA clone 339305 3′). The predicted amino acid sequence disclosed herein for AT340_{—}1 was searched against the GenPept and GeneSeq amino acid sequence databases using the BLASTX search protocol. The predicted AT340_{—}1 protein demonstrated at least some similarity to sequences identified as U21324 (similar to S. cerevisiae hypothetical protein YKL166 [Caenorhabditis elegans]).

Based upon sequence similarity, AT340_{—}1 proteins and each similar protein or peptide may share at least some activity.

Clone “BG132_{—}1”

A polynucleotide of the present invention has been identified as clone “BG132_{—}1”. BG132_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino add sequence of the encoded protein. BG132_{—}1 is a full-length clone, including the entire coding sequence of a secreted protein (also referred to herein as “BG132_{—}1 protein”).

The nucleotide sequence of the 5′ portion of BG132_{—}1 as presently determined is reported in SEQ ID NO:116. What applicants presently believe is the proper reading frame for the coding region is indicated in SEQ ID NO:117. The predicted amino acid sequence of the BG132_{—}1 protein corresponding to the foregoing nucleotide sequence is reported in SEQ ID NO:117. Amino acids 121 to 133 of SEQ ID NO:117 are a predicted leader/signal sequence, with the predicted mature amino acid sequence beginning at amino acid 134. Due to the hydrophobic nature of the predicted leader/signal sequence, it is likely to act as a transmembrane domain should the predicted leader/signal sequence not be separated from the remainder of the BG132_{—}1 protein. Additional nucleotide sequence from the 3′ portion of BG132_{—}1, including a poly(A) tail, is reported in SEQ ID NO:118.

The EcoRI/NotI restriction fragment obtainable from the deposit containing clone BG132_{—}1 should be approximately 2000 bp.

The nucleotide sequence disclosed herein for BG132_{—}1 was searched against the GenBank and GeneSeq nucleotide sequence databases using BLASTN/BLASTX and FASTA search protocols. BG132_{—}1 demonstrated at least some similarity with sequences identified as AA078587 (7P05H12 Chromosome 7 Placental cDNA Library Homo sapiens cDNA clone 7P05H12), H14301 (ym63c04.r1 Homo sapiens cDNA clone 163590 5′ similar to gb:U03642_cds1 PROBABLE G PROTEIN-COUPLED RECEPTOR APJ (HUMAN)), L09249 (putative G-protein coupled receptor, rhodopsin family), S79811 (adrenomedullin receptor [rats, lung, mRNA]), T36034 (rchd523 gene differentially expressed in cardiovascular disease), U58828 (Human IL8-related receptor (DRY12) mRNA, complete cds), and Y08162 (H. sapiens mRNA for heptahelix receptor). The predicted amino add sequence disclosed herein for BG132_{—}1 was searched against the GenPept and GeneSeq amino acid sequence databases using the BLASTX search protocol. The predicted BG132_{—}1 protein demonstrated at least some similarity to sequences identified as L06109 (G protein-coupled receptor [Gallus gallus]), L34339 (galanin receptor [Homo sapiens]), U30290 (galanin receptor GALR1 [Rattus norvegicus]), U58828 (IL8-related receptor [Homo sapiens]), WO3739 (rchd523 gene product (G protein-coupled receptor)), X98510 (G protein-coupled receptor [Homo sapiens]), and Y08162 (heptahelix receptor [Homo sapiens]). Based upon sequence similarity, BG132_{—1} proteins and each similar protein or peptide may share at least some activity.

Clone “BG219_{—}2”

A polynucleotide of the present invention has been identified as clone “BG219_{—}2”. BG219_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG219_{—}2 is a full-length done, including the entire coding sequence of a secreted protein (also referred to herein as “BG219_{—}2 protein”).

The nucleotide sequence of BG219_{—}2 as presently determined is reported in SEQ ID NO:119, and includes a poly(A) tail. What applicants presently believe to be the proper reading frame and the predicted amino acid sequence of the BG219_{—}2 protein corresponding to the foregoing nucleotide sequence is reported in SEQ ID NO:120.

The EcoRI/NotI restriction fragment obtainable from the deposit containing clone BG219_{—}2 should be approximately 700 bp.

The nucleotide sequence disclosed herein for BG219_{—}2 was searched against the GenBank and GeneSeq nucleotide sequence databases using BLASTN/BLASTX and FASTA search protocols. BG219_{—}2 demonstrated at least some similarity with sequences identified as AA210695 (zr88b05.s1 Soares NbHTGBC Homo sapiens cDNA clone 682737 3′), C01459 (HUMGS0008450, Human Gene Signature, 3′-directed cDNA sequence), N22628 (EST49p115 Homo sapiens cDNA clone 49p115), and T26211 (Human gene signature HUMGS08450). Based upon sequence similarity, BG219 2 proteins and each similar protein or peptide may share at least some activity.

Clone “BG366_{—}2”

A polynucleotide of the present invention has been identified as clone “BG366_{—}2”. BG366_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG366_{—}2 is a full-length done, including the entire coding sequence of a secreted protein (also referred to herein as “BG366 2 protein”).

The nucleotide sequence of BG366_{—}2 as presently determined is reported in SEQ ID NO:121, and includes a poly(A) tail. What applicants presently believe to be the proper reading frame and the predicted amino add sequence of the BG366_{—}2 protein corresponding to the foregoing nucleotide sequence is reported in SEQ ID NO:122. The amino acid sequence of another protein that could be encoded by BG366_{—}2 is reported in SEQ ID NO:283.

The EcoRI/NotI restriction fragment obtainable from the deposit containing clone BG366_{—}2 should be approximately 3000 bp.

The nucleotide sequence disclosed herein for BG366_{—}2 was searched against the GenBank and GeneSeq nucleotide sequence databases using BLASTN/BLASTX and FASTA search protocols. BG366_{—}2 demonstrated at least some similarity with sequences identified as N39453 (yy49h03.s1 Homo sapiens cDNA clone 276917 3′). Based upon sequence similarity, BG366_{—}2 proteins and each similar protein or peptide may share at least some activity. The TopPredII computer program predicts a potential transmembrane domain within the BG366_{—}2 protein sequence centered around amino acid 92 of SEQ ID NO:122.

Clone “BV172_{—}2”

A polynucleotide of the present invention has been identified as clone “BV172_{—}2′. BV172_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV172_{—}2 is a full-length clone, including the entire coding sequence of a secreted protein (also referred to herein as “BV172_{—}2 protein”).

The nucleotide sequence of BV172_{—}2 as presently determined is reported in SEQ ID NO:123, and includes a poly(A) tail. What applicants presently believe to be the proper reading frame and the predicted amino acid sequence of the BV172_{—}2 protein corresponding to the foregoing nucleotide sequence is reported in SEQ ID NO:124.

The EcoRI/NotI restriction fragment obtainable from the deposit containing clone BV172_{—}2 should be approximately 1650 bp.

The nucleotide sequence disclosed herein for BV172_{—}2 was searched against the GenBank and GeneSeq nucleotide sequence databases using BLASTN/BLASTX and FASTA search protocols. BV172_{—}2 demonstrated at least some similarity with sequences identified as No significant hits were found in the database. The TopPredII computer program predicts a potential transmembrane domain within the BV172_{—}2 protein sequence centered around amino acid 19 of SEQ ID NO:124. The nucleotide sequence of BV172_{—}2 indicates that it may contain one or more of the following types of repetitive elements: an element similar to chicken CR1, human L1, Mer33.

Clone “CC247_{—}10”

A polynucleotide of the present invention has been identified as clone “CC247_{—}10”. CC247_{—}10 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC247_{—}10 is a full-length done, including the entire coding sequence of a secreted protein (also referred to herein as “CC247_{—}10 protein”).

The nucleotide sequence of CC47_{—}10 as presently determined is reported in SEQ ID NO:125, and includes a poly(A) tail. What applicants presently believe to be the proper reading frame and the predicted amino acid sequence of the CC247_{—}10 protein corresponding to the foregoing nucleotide sequence is reported in SEQ ID NO:126. Amino acids 1 to 8 of SEQ ID NO:126 are a predicted leader/signal sequence, with the predicted mature amino acid sequence beginning at amino acid 9. Due to the hydrophobic nature of the predicted leader/signal sequence, it is likely to act as a transmembrane domain should the predicted leader/signal sequence not be separated from the remainder of the CC247_{—}10 protein.

The EcoRI/NotI restriction fragment obtainable from the deposit containing clone CC247_{—}10 should be approximately 550 bp. The nucleotide sequence disclosed herein for CC247_{—}10 was searched against the GenBank and GeneSeq nucleotide sequence databases using BLASTN/BLASTX and FASTA search protocols. CC247_{—}10 demonstrated at least some similarity with sequences identified as AA291226 (zs47d03x1 NCl_CGAP_GCB1 Homo sapiens cDNA clone 700613 5′), T05738 (EST03627 Homo sapiens cDNA clone HFBDF64), W51195 (ma14b04.r1 Life Tech mouse brain Mus musculus cDNA clone 304495 5′), and W93640 (zd95d09.s1 Soares fetal heart NbHH19W Homo sapiens cDNA clone 357233 3′). The predicted amino acid sequence disclosed herein for CC247_{—}10 was searched against the GenPept and GeneSeq amino acid sequence databases using the BLASTX search protocol. The predicted CC247_{—}0 protein demonstrated at least some similarity to sequences identified as M62424 (thrombin receptor [Homo sapiens]). The predicted CC247_{—}10 protein is highly hydrophobic. Based upon sequence similarity, CC247_{—}10 proteins and each similar protein or peptide may share at least some activity.

Clone “CI480_{—}9”

A polynucleotide of the present invention has been identified as clone “CI480_{—}9”. CI48_{—}9 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CI480_{—}9 is a full-length clone, including the entire coding sequence of a secreted protein (also referred to herein as “CI480_{—}9 protein”).

The nucleotide sequence of CI480_{—}9 as presently determined is reported in SEQ ID NO:127, and includes a poly(A) tail. What applicants presently believe to be the proper reading frame and the predicted amino acid sequence of the CI480_{—}9 protein corresponding to the foregoing nucleotide sequence is reported in SEQ ID NO:128. Amino acids 39 to 51 of SEQ ID NO:128 are a predicted leader/signal sequence, with the predicted mature amino acid sequence beginning at amino acid 52. Due to the hydrophobic nature of the predicted leader/signal sequence, it is likely to act as a transmembrane domain should the predicted leader/signal sequence not be separated from the remainder of the CI480_{—}9 protein.

The EcoRI/NotI restriction fragment obtainable from the deposit containing clone CI480_{—}9 should be approximately 1940 bp. The nucleotide sequence disclosed herein for CI480_{—}9 was searched against the GenBank and GeneSeq nucleotide sequence databases using BLASTN/BLASTX and FASTA search protocols. CI480_{—}9 demonstrated at least some similarity with sequences identified as N99342 (IMAGE:20093 Homo sapiens cDNA clone 20093), R89725 (ym99d09.r1 Homo sapiens cDNA clone 167057 5′), and U60644 (Human HU-K4 mRNA, complete cds). The predicted amino acid sequence disclosed herein for CI480_{—}9 was searched against the GenPept and GeneSeq amino acid sequence databases using the BLASTX search protocol. The predicted CI480_{—}9 protein demonstrated at least some similarity to sequences identified as U60644 (HU-K4 [Homo sapiens]). Based upon sequence similarity, CI480_{—}9 proteins and each similar protein or peptide may share at least some activity.

CI480_{—}9 protein was expressed in a COS cell expression system, and an expressed protein band of approximately 63 kDa was detected in membrane fractions using SDS polyacrylamide gel electrophoresis.

Clone “CO722_{—}1”

A polynucleotide of the present invention has been identified as clone “CO722_{—}1”. CO722_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CO722_{—}1 is a full-length clone, including the entire coding sequence of a secreted protein (also referred to herein as “CO722_{—}1 protein”).

The nucleotide sequence of CO722_{—}1 as presently determined is reported in SEQ ID NO:129, and includes a poly(A) tail. What applicants presently believe to be the proper reading frame and the predicted amino acid sequence of the C0722_{—}1 protein corresponding to the foregoing nucleotide sequence is reported in SEQ ID NO:130. Amino acids 17 to 29 of SEQ ID NO:130 are a predicted leader/signal sequence, with the predicted mature amino acid sequence beginning at amino acid 30. Due to the hydrophobic nature of the predicted leader/signal sequence, it is likely to act as a transmembrane domain should the predicted leader/signal sequence not be separated from the remainder of the C0722_{—}1 protein.

The EcoRI/NotI restriction fragment obtainable from the deposit containing clone CO722_{—}1 should be approximately 6800 bp.

The nucleotide sequence disclosed herein for CO722_{—}1 was searched against the GenBank and GeneSeq nucleotide sequence databases using BLASTN/BLASTX and FASTA search protocols. CO722_{—}1 demonstrated at least some similarity with sequences identified as AA186616 (zp71a08.s1 Stratagene endothelial cell 937223 Homo sapiens cDNA clone 625622 3′ similar to contains Alu repetitive element), H10376 (ym08a03.s1 Homo sapiens cDNA clone 47067 3′), N86013 (J5997F Fetal heart, Lambda ZAP Express Homo sapiens cDNA), U55258 (Human hBRAVO/Nr-CAM precursor (hBRAVO/Nr-CAM) gene, complete cds), W19770 (zb39d01.r1 Soares parathyroid tumor NbHPA Homo sapiens), W31608 (zb91d09.r1 Soares parathyroid tumor NbHPA Homo sapiens cDNA clone), and X58482 (Chicken mRNA for neuronal transmembrane protein Nr-CAM, ng-CAM related). The predicted amino acid sequence disclosed herein for CO722_{—}1 was searched against the GenPept and GeneSeq amino acid sequence databases using the BLASTX search protocol. The predicted CO722_protein demonstrated at least some similarity to sequences identified as AB002341 (KIAA0343 [Homo sapiens]) and X58482 (Nr-CAM protein [Gallus gallus]). Based upon sequence similarity, CO722_{—}1 proteins and each similar protein or peptide may share at least some activity. The TopPredII computer program predicts two potential transmembrane domains within the CO722_{—}1 protein sequence, centered around amino acids 610 and 1070 of SEQ ID NO:130, respectively.

CO722_{—}1 protein was expressed in a COS cell expression system, and an expressed protein band of approximately 160 kDa was detected in conditioned media fractions using SDS polyacrylamide gel electrophoresis.

Clone “CT748_{—}2”

A polynucleotide of the present invention has been identified as clone “CT748_{—}2”.

CT748_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CT748_{—}2 is a full-length done, including the entire coding sequence of a secreted protein (also referred to herein as “CT748_{—}2 protein”).

The nucleotide sequence of CT748_{—}2 as presently determined is reported in SEQ ID NO:131, and includes a poly(A) tail. What applicants presently believe to be the proper reading frame and the predicted amino acid sequence of the CT748_{—}2 protein corresponding to the foregoing nucleotide sequence is reported in SEQ ID NO:132. Amino acids 281 to 293 of SEQ ID NO:132 are a predicted leader/signal sequence, with the predicted mature amino acid sequence beginning at amino acid 294. Due to the hydrophobic nature of the predicted leader/signal sequence, it is likely to act as a transmembrane domain should the predicted leader/signal sequence not be separated from the remainder of the CT748_{—}2 protein.

The EcoRI/NotI restriction fragment obtainable from the deposit containing clone CT748_{—}2 should be approximately 5500 bp.

The nucleotide sequence disclosed herein for CT748_{—}2 was searched against the GenBank and GeneSeq nucleotide sequence databases using BLASTN/BLASTX and FASTA search protocols. CT748_{—}2 demonstrated at least some similarity with sequences identified as T48063 (yb24f03.s1 Homo sapiens cDNA clone 72125 3′) and X54175 (Human specific Alu element (HS C4N2) DNA). The predicted amino add sequence disclosed herein for CT748_{—}2 was searched against the GenPept and GeneSeq amino acid sequence databases using the BLASTX search protocol. The predicted CT748_{—}2 protein demonstrated at least some similarity to sequences identified as Z36714 (cyclin F [Homo sapiens]). Based upon sequence similarity, CT748_{—}2 proteins and each similar protein or peptide may share at least some activity. The nucleotide sequence of CT748_{—}2 indicates that it may contain an Alu repetitive element.

Clone “AT1_{—}1”

A polynucleotide of the present invention has been identified as clone “AJ1_{—}1”.

AJ1_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AJ1_{—}1 is a full-length clone, including the entire coding sequence of a secreted protein (also referred to herein as “AJ1_{—}1 protein”).

The nucleotide sequence of the 5′ portion of AJ1_{—}1 as presently determined is reported in SEQ ID NO:133. What applicants presently believe is the proper reading frame for the coding region is indicated in SEQ ID NO:134. The predicted amino acid sequence of the AJ1_{—}1 protein corresponding to the foregoing nucleotide sequence is reported in SEQ ID NO:134. Additional nucleotide sequence from the 3′ portion of AJ1_{—}1, including a poly(A) tail, is reported in SEQ ID NO:135.

The EcoRI/NotI restriction fragment obtainable from the deposit containing clone AJ1_{—}1 should be approximately 925 bp.

The predicted amino acid sequence disclosed herein for AJ1_{—}1 was searched against the GenPept and GeneSeq amino acid sequence databases using the BLASTX search protocol. The predicted AJ1_{—}1 protein demonstrated at least some similarity to sequences identified as U39060 (GRIP1 [Mus musculus]). Based upon sequence similarity, AJ1_{—}1 proteins and each similar protein or peptide may share at least some activity.

Clone “AO73_{—}3”

A polynucleotide of the present invention has been identified as clone “AQ73_{—}3”. AQ73_{—}3 was isolated from a human adult ovary (PA-1 teratocarcinoma, untreated tissue pooled with retinoic-acid-treated and activin-treated tissue) cDNA library using methods which are selective for cDNAs encoding secreted proteins (see U.S. Pat. No. 5,536,537), or was identified as encoding a secreted or transmembrane protein on the basis of computer analysis of the amino acid sequence of the encoded protein. AQ73_{—}3 is a full-length done, including the entire coding sequence of a secreted protein (also referred to herein as “AQ73_{—}3 protein”).

The nucleotide sequence of AQ73_{—}3 as presently determined is reported in SEQ ID NO:136, and includes a poly(A) tail. What applicants presently believe to be the proper reading frame and the predicted amino acid sequence of the AQ73_{—}3 protein corresponding to the foregoing nucleotide sequence is reported in SEQ ID NO:137.

The EcoRI/NotI restriction fragment obtainable from the deposit containing clone AQ73_{—}3 should be approximately 2800 bp.

The nucleotide sequence disclosed herein for AQ73_{—}3 was searched against the GenBank and GeneSeq nucleotide sequence databases using BLASTN/BLASTX and FASTA search protocols. AQ73_{—}3 demonstrated at least some similarity with sequences identified as AA514474 (nf57g01.s1 NCI_CGAP_Co3 Homo sapiens cDNA clone 924048), T47520 (Human hepatoma-derived growth factor (HDGF-2) cDNA), W24708 (zb62eO8.rl Soares fetal lung NbHL19W Homo sapiens cDNA clone 308198 5′), and W45513 (zc27g08.s1 Soares senescent fibroblasts NbHSF Homo sapiens cDNA clone 323582 3′). The predicted amino acid sequence disclosed herein for AQ73_{—}3 was searched against the GenPept and GeneSeq amino acid sequence databases using the BLASTX search protocol. The predicted AQ73-3 protein demonstrated at least some similarity to sequences identified as D16431 (hepatoma-derived GF [Homo sapiens]), D63707 (mouse hepatoma derived growth factor (HDGF) [Mus musculus]), R66727 (Human hepatoma derived growth factor), U18997 (ORF_{—}299 [Escherichia coli]), U97193 (similar to S. cerevisiae SIR2 (SP P06700) and mouse hepatoma derived growth factor HDGF (NID g945418) [Caenorhabditis elegans]), and WO9404 (Human hepatoma-derived growth factor (HDGF-2)). Based upon sequence similarity, AQ73_{—}3 proteins and each similar protein or peptide may share at least some activity.

AQ73_{—}3 protein was expressed in a COS cell expression system, and an expressed protein band of approximately 67 kDa was detected in membrane fractions using SDS polyacrylamide gel electrophoresis.

Clone “BG142_{—}1”

A polynucleotide of the present invention has been identified as clone “BG142_{—}1”. BG142_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG142_{—}1 is a full-length clone, including the entire coding sequence of a secreted protein (also referred to herein as “BG142_{—}1 protein”).

The nucleotide sequence of BG142_{—}1 as presently determined is reported in SEQ ID NO:138, and includes a poly(A) tail. What applicants presently believe to be the proper reading frame and the predicted amino acid sequence of the BG142_{—}1 protein corresponding to the foregoing nucleotide sequence is reported in SEQ ID NO:139.

The EcoRI/NotI restriction fragment obtainable from the deposit containing clone BG142_{—}1 should be approximately 1100 bp. The nucleotide sequence disclosed herein for BG142_{—}1 was searched against the GenBank and GeneSeq nucleotide sequence databases using BLASTN/BLASTX and FASTA search protocols. BG142_{—}1 demonstrated at least some similarity with sequences identified as AA170261 (ms87h11.r1 Soares mouse 3NbMS Mus musculus cDNA clone 618597 5′ similar to TR E245601 E245601 G-RICH BOX-BINDING PROTEIN), L04282 (Human CACCC box-binding protein mRNA, complete cds), N27696 (yx51h12.r1 Homo sapiens cDNA clone 265319 5′), W96110 (ze09a11.r1 Soares fetal heart NbHH19W Homo sapiens cDNA clone 358460 5′), and W96111 (ze09a11.s1 Soares fetal heart NbHH19W Homo sapiens cDNA clone 358460 3′). The predicted amino acid sequence disclosed herein for BG142_{—}1 was searched against the GenPept and GeneSeq amino acid sequence databases using the BLASTX search protocol. The predicted BG142_{—}1 protein demonstrated at least some similarity to sequences identified as U80078 (transcription factor BFCOL1 [Mus musculus]) and X98096 (G-rich box-binding protein [Mus musculus]). Based upon sequence similarity, BG142_{—}1 proteins and each similar protein or peptide may share at least some activity.

Clone “BV66_{—}1”

A polynucleotide of the present invention has been identified as clone “BV66_{—}1”. BV66_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV66_{—}1 is a full-length clone, including the entire coding sequence of a secreted protein (also referred to herein as “BV66_{—}1 protein”).

The nucleotide sequence of BV66_{—}1 as presently determined is reported in SEQ ID NO:140, and includes a poly(A) tail. What applicants presently believe to be the proper reading frame and the predicted amino acid sequence of the BV66_{—}1 protein corresponding to the foregoing nucleotide sequence is reported in SEQ ID NO:141.

The EcoRI/NotI restriction fragment obtainable from the deposit containing clone BV66_{—}1 should be approximately 870 bp.

The nucleotide sequence disclosed herein for BV66_{—}1 was searched against the GenBank and GeneSeq nucleotide sequence databases using BLASTN/BLASTX and FASTA search protocols. No hits were found in the database. The nucleotide sequence of BV66_{—}1 indicates that it may contain a TAAA1 simple repeat element.

Clone “BV291_{—}3”

A polynucleotide of the present invention has been identified as clone “BV291_{—}3”. BV291_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV291_{—}3 is a full-length clone, including the entire coding sequence of a secreted protein (also referred to herein as “BV291_{—}3 protein”).

The nucleotide sequence of BV291_{—}3 as presently determined is reported in SEQ ID NO:142, and includes a poly(A) tail. What applicants presently believe to be the proper reading frame and the predicted amino acid sequence of the BV291_{—}3 protein corresponding to the foregoing nucleotide sequence is reported in SEQ ID NO:143.

The EcoRI/NotI restriction fragment obtainable from the deposit containing clone BV291_{—}3 should be approximately 2000 bp.

The nucleotide sequence disclosed herein for BV291_{—}3 was searched against the GenBank and GeneSeq nucleotide sequence databases using BLASTN/BLASTX and FASTA search protocols. BV291_{—}3 demonstrated at least some similarity with sequences identified as H10954 (ym06e09.r1 Homo sapiens cDNA clone 47034 5′), H10955 (ym06e09.s1 Homo sapiens cDNA clone 47034 3′), N25300 (yw52c10.s1 Homo sapiens cDNA clone 255858 3′), T25940 (Human gene signature HUMGS08173), T68890 (yc30g11.s1 Homo sapiens cDNA clone 82244 3′), T78286 (yc99a08.r1 Homo sapiens cDNA clone 24033 5′), Z39987 (H. sapiens partial cDNA sequence; clone c-1oh05), and Z47073 (Caenorhabditis elegans cosmid ZC506). The predicted amino acid sequence disclosed herein for BV291_{—}3 was searched against the GenPept and GeneSeq amino acid sequence databases using the BLASTX search protocol. The predicted BV291_{—}3 protein demonstrated at least some similarity to sequences identified as X02155 (BTTGR_{—}1 thyroglobulin [Bos taurus]). Based upon sequence similarity, BV291_{—}3 proteins and each similar protein or peptide may share at least some activity. The TopPredII computer program predicts a potential transmembrane domain within the BV291_{—}3 protein sequence centered around amino acid 48 of SEQ ID NO:143.

BV291_{—}3 protein was expressed in a COS cell expression system, and an expressed protein band of approximately 30 kDa was detected in conditioned medium using SDS polyacrylamide gel electrophoresis.

Clone “CK201_{—}1”

A polynucleotide of the present invention has been identified as clone “CK201_{—}1”. CK201_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CK201_{—}1 is a full-length clone, including the entire coding sequence of a secreted protein (also referred to herein as “CK201_{—}1 protein”). The nucleotide sequence of CK201_{—}1 as presently determined is reported in SEQ ID NO:144, and includes a poly(A) tail. What applicants presently believe to be the proper reading frame and the predicted amino acid sequence of the CK201_{—}1 protein corresponding to the foregoing nucleotide sequence is reported in SEQ ID NO:145.

The EcoRI/NotI restriction fragment obtainable from the deposit containing clone CK201_{—}1 should be approximately 1080 bp.

The nucleotide sequence disclosed herein for CK201_{—}1 was searched against the GenBank and GeneSeq nucleotide sequence databases using BLASTN/BLASTX and FASTA search protocols. CK201_{—}1 demonstrated at least some similarity with sequences identified as AA129133 (zo09h12.s1 Stratagene neuroepithelium NT2RAMI 937234 Homo sapiens cDNA clone 567239 3′ similar to contains Alu repetitive element), D81444 (Human fetal brain cDNA 5′ end GEN-164G10), R36326 (yg69h09.r1 Homo sapiens cDNA clone 38821 5′), T08553 (Oncogene R-ras mutant cDNA (exons 2-6)), T31595 (Probe (BLUR13) for Alu repeat sequence), X03273 (Human Alu-family cluster 5′ of alpha(1)-acid glycoprotein gene), and X69907 (H. sapiens gene for mitochondrial ATP synthase c subunit). The predicted amino acid sequence disclosed herein for CK201_{—}1 was searched against the GenPept and GeneSeq amino acid sequence databases using the BLASTX search protocol. The predicted CK201_{—}1 protein demonstrated at least some similarity to sequences identified as D21827 (major surface glycoprotein [Pneumocystis carinii]). Based upon sequence similarity, CK201_{—}1 proteins and each similar protein or peptide may share at least some activity. The nucleotide sequence of CK201_{—}1 indicates that it may contain an Alu repetitive element.

CK201_{—}1 protein was expressed in a COS cell expression system, and an expressed protein band of approximately 40 kDa was detected in conditioned medium and membrane fractions using SDS polyacrylamide gel electrophoresis.

Clone “CO331_{—}2”

A polynucleotide of the present invention has been identified as clone “CQ331_{—}2”. CQ331_{—}2 was isolated from a human adult heart cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CQ331_{—}2 is a full-length clone, including the entire coding sequence of a secreted protein (also referred to herein as “CQ331_{—}2 protein”). The nucleotide sequence of CQ331_{—}2 as presently determined is reported in SEQ ID NO:146, and includes a poly(A) tail. What applicants presently believe to be the proper reading frame and the predicted amino acid sequence of the CQ331_{—}2 protein corresponding to the foregoing nucleotide sequence is reported in SEQ ID NO:147. Amino acids 7 to 19 of SEQ ID NO:147 are a predicted leader/signal sequence, with the predicted mature amino add sequence beginning at amino add 20. Due to the hydrophobic nature of the predicted leader/signal sequence, it is likely to act as a transmembrane domain should the predicted leader/signal sequence not be separated from the remainder of the CQ331_{—}2 protein.

The EcoRI/NotI restriction fragment obtainable from the deposit containing clone CQ331_{—}2 should be approximately 1600 bp.

The nucleotide sequence disclosed herein for CQ331_{—}2 was searched against the GenBank and GeneSeq nucleotide sequence databases using BLASTN/BLASTX and FASTA search protocols. CQ331_{—}2 demonstrated at least some similarity with sequences identified as J03766 (Canine cardiac calsequestrin mRNA, complete cds), L29766 (Homo sapiens epoxide hydrolase (EPHX) gene, complete cds), N83601 (KK1173F Homo sapiens cDNA clone KK1173 5′ similar to CALSEQUESTRIN (CARDIAC)), T99646 (ye73f12.s1 Homo sapiens cDNA clone 123407 3′ similar to contains Alu repetitive element; contains PTR5 repetitive element), and W76326 (zd60d04.r1 Soares fetal heart NbHH19W Homo sapiens cDNA clone 345031 5′ similar to contains Alu repetitive element). The predicted amino acid sequence disclosed herein for CQ331_{—}2 was searched against the GenPept and GeneSeq amino acid sequence databases using the BLASTX search protocol. The predicted CQ331_{—}2 protein demonstrated at least some similarity to sequences identified as J03766 (DOGCAL_{—}1 Canine cardiac calsequestrin mRNA, complete cds [Canis canis]) and X55040 (calsequestrin [Oryctolagus cuniculus]). Based upon sequence similarity, CQ331_{—}2 proteins and each similar protein or peptide may share at least some activity.

Clone “CT550_{—}1”

A polynucleotide of the present invention has been identified as clone “CT550_{—}1”. CT550_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CT550_{—}1 is a full-length clone, including the entire coding sequence of a secreted protein (also referred to herein as “CT550_{—}1 protein”).

The nucleotide sequence of CT550_{—}1 as presently determined is reported in SEQ ID NO:148, and includes a poly(A) tail. What applicants presently believe to be the proper reading frame and the predicted amino acid sequence of the CT550_{—}1 protein corresponding to the foregoing nucleotide sequence is reported in SEQ ID NO:149. The EcoRI/NotI restriction fragment obtainable from the deposit containing clone CT550_{—}1 should be approximately 1070 bp.

The nucleotide sequence disclosed herein for CT550_{—}1 was searched against the GenBank and GeneSeq nucleotide sequence databases using BLASTN/BLASTX and FASTA search protocols. No hits were found in the database. The TopPredII computer program predicts a potential transmembrane domain within the CT550_{—}1 protein sequence centered around amino acid 25 of SEQ ID NO:149.

CT550_{—}1 protein was expressed in a COS cell expression-system, and an expressed protein band of approximately 7 kDa was detected in membrane fractions using SDS polyacrylamide gel electrophoresis.

Clone “CT585_{—}1”

A polynucleotide of the present invention has been identified as clone “CT585_{—}1”. CT585_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CT585_{—}1 is a full-length clone, including the entire coding sequence of a secreted protein (also referred to herein as “CT585_{—}1 protein”).

The nucleotide sequence of CT585_{—}1 as presently determined is reported in SEQ ID NO:150, and includes a poly(A) tail. What applicants presently believe to be the proper reading frame and the predicted amino acid sequence of the CT585_{—}1 protein corresponding to the foregoing nucleotide sequence is reported in SEQ ID NO:151. Amino acids 2 to 14 of SEQ ID NO:151 are a predicted leader/signal sequence, with the predicted mature amino acid sequence beginning at amino acid 15. Due to the hydrophobic nature of the predicted leader/signal sequence, it is likely to act as a transmembrane domain should the predicted leader/signal sequence not be separated from the remainder of the CT585_{—}1 protein.

The EcoRI/NotI restriction fragment obtainable from the deposit containing clone CT585_{—}1 should be approximately 2710 bp.

The nucleotide sequence disclosed herein for CT585_{—}1 was searched against the GenBank and GeneSeq nucleotide sequence databases using BLASTN/BLASTX and FASTA search protocols. CT585_{—}1 demonstrated at least some similarity with sequences identified as AA069442 (zf74b02.s1 Soares pineal gland N3HPG Homo sapiens cDNA clone 382635 3′), L38961 (Homo sapiens putative transmembrane protein (B5) mRNA, complete cds), N34932 (yy49b10.s1 Homo sapiens cDNA clone 276859 3′), N60101 (TgESTzy11f10.r1 Toxoplasma gondii cDNA clone tgzy11f10.r1 5′), and U13019 (Caenorhabditis elegans cosmid T12A2). The predicted amino acid sequence disclosed herein for CT585_{—}1 was searched against the GenPept, GeneSeq, and SwissProt amino acid sequence databases using the BLASTX search protocol. The predicted CT585_{—}1 protein demonstrated at least some similarity to sequences identified as L34260 (transmembrane protein [Mus musculus]), L38961 (transmembrane protein [Homo sapiens]), P46975 (Caenorhabditis elegans oligosaccharyl transferase stt3 [Caenorhabditis elegans]), and U13019 (Caenorhabditis elegans cosmid T12A2 [Caenorhabditis elegans]). Based upon sequence similarity, CT585_{—}1 proteins and each similar protein or peptide may share at least some activity.

Clone “CT797_{—}3”

A polynucleotide of the present invention has been identified as clone “CT797_{—}3”.

CT797_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CT797_{—}3 is a full-length clone, including the entire coding sequence of a secreted protein (also referred to herein as “CT797_{—}3 protein”).

The nucleotide sequence of CT797_{—}3 as presently determined is reported in SEQ ID NO:152, and includes a poly(A) tail. What applicants presently believe to be the proper reading frame and the predicted amino acid sequence of the CT797_{—}3 protein corresponding to the foregoing nucleotide sequence is reported in SEQ ID NO:153.

The EcoRI/NotI restriction fragment obtainable from the deposit containing clone CT797_{—}3 should be approximately 3300 bp.

The nucleotide sequence disclosed herein for CT797_{—}3 was searched against the GenBank and GeneSeq nucleotide sequence databases using BLASTN/BLASTX and FASTA search protocols. CT797_{—}3 demonstrated at least some similarity with sequences identified as AA573847 (nk08d06.s1 NCl_CGAP_Co2 Homo sapiens cDNA clone IMAGE:1012907). The predicted amino acid sequence disclosed herein for CT797_{—}3 was searched against the GenPept and GeneSeq amino acid sequence databases using the BLASTX search protocol. The predicted CT797_{—}3 protein demonstrated at least some similarity to sequences identified as U18309 (chromokinesin [Gallus gallus]) and Z82271 (T01G1.1 [Caenorhabditis elegans]). Based upon sequence similarity, CT797_{—}3 proteins and each similar protein or peptide may share at least some activity.

CT797_{—}3 protein was expressed in a COS cell expression system, and an expressed protein band of approximately 80 kDa was detected in conditioned medium using SDS polyacrylamide gel electrophoresis.

Clone “CB107_{—}1”

A polynucleotide of the present invention has been identified as clone “CB107_{—}1”. CB107_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CB107_{—}1 is a full-length clone, including the entire coding sequence of a secreted protein (also referred to herein as “CB107_{—}1 protein”).

The nucleotide sequence of the 5′ portion of CB107_{—}1 as presently determined is reported in SEQ ID NO:154. An additional internal nucleotide sequence from CB107_{—}1 as presently determined is reported in SEQ ID NO:155. What applicants believe is the proper reading frame and the predicted amino acid sequence encoded by such internal sequence is reported in SEQ ID NO:156. Additional nucleotide sequence from the 3′ portion of CB107_{—}1, including a poly(A) tail, is reported in SEQ ID NO:157.

The EcoRI/NotI restriction fragment obtainable from the deposit containing clone CB107_{—}1 should be approximately 3300 bp.

The nucleotide sequence disclosed herein for CB107_{—}1 was searched against the GenBank and GeneSeq nucleotide sequence databases using BLASTN/BLASTX and FASTA search protocols. CB107_{—}1 demonstrated at least some similarity with sequences identified as AA121485 (zn80a02.s1 Stratagene lung carcinoma 937218 Homo sapiens cDNA clone 564458 3′), AA428192 (zw51b08.s1 Soares total fetus Nb2HF8 9w Homo sapiens cDNA clone 773559 3′), D83018 (Human mRNA for nel-related protein 2, complete cds), F10919 (H. sapiens partial cDNA sequence; clone c-31g11), H15375 (ym28d09.r1 Homo sapiens cDNA clone 49527 5′ similar to SP A54105 A54105 FIBRILLIN-2 PRECURSOR), U48245 (Rattus norvegicus protein kinase C-binding protein Nel mRNA, complete cds), U59230 (Mus musculus mel (MEL91) mRNA, complete cds), and W28387 (46c5 Human retina cDNA randomly primed sublibrary Homo sapiens cDNA). The predicted amino acid sequence disclosed herein for CB07_{—}1 was searched against the GenPept and GeneSeq amino add sequence databases using the BLASTX search protocol. The predicted CB107_{—}1 protein demonstrated at least some similarity to sequences identified as D83018 (nel-related protein 2 [Homo sapiens]), R05222 (Antigen GX5401FL encoded by Eimeria tenella genomic DNA), R79964 (Connective tissue growth factor), U48245 (RNU48245_{—}1 protein kinase C-binding protein Nel [Rattus norvegicus]), and U59230 (mel [Mus musculus]). Based upon sequence similarity, CB107_{—}1 proteins and each similar protein or peptide may share at least some activity.

Clone “CG300_{—}3”

A polynucleotide of the present invention has been identified as clone “CG300_{—}3”. CG300_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CG300_{—}3 is a full-length clone, including the entire coding sequence of a secreted protein (also referred to herein as “CG300_{—}3 protein”).

The nucleotide sequence of CG300_{—}3 as presently determined is reported in SEQ ID NO:158, and includes a poly(A) tail. What applicants presently believe to be the proper reading frame and the predicted amino acid sequence of the CG300_{—}3 protein corresponding to the foregoing nucleotide sequence is reported in SEQ ID NO:159. Amino adds 30 to 42 of SEQ ID NO:159 are a predicted leader/signal sequence, with the predicted mature amino acid sequence beginning at amino acid 43. Due to the hydrophobic nature of the predicted leader/signal sequence, it is likely to act as a transmembrane domain should the predicted leader/signal sequence not be separated from the remainder of the CG300_{—}3 protein.

The EcoRI/NotI restriction fragment obtainable from the deposit containing clone CG300_{—}3 should be approximately 1800 bp. The nucleotide sequence disclosed herein for CG300_{—}3 was searched against the GenBank and GeneSeq nucleotide sequence databases using BLASTN/BLASTX and FASTA search protocols. CG300_{—}3 demonstrated at least some similarity with sequences identified as N40185 (yy44d08.s1 Homo sapiens cDNA clone 276399 3′) and WO1791 (za72d06.r1 Soares fetal lung NbHL19W Homo sapiens cDNA clone 298091 5′). Based upon sequence similarity, CG300_{—}3 proteins and each similar protein or peptide may share at least some activity. The TopPredII computer program predicts four potential transmembrane domains within the CG300_{—}3 protein sequence, centered around amino acids 34, 98, 151, and 179 of SEQ ID NO:159, respectively.

CG300_{—}3 protein was expressed in a COS cell expression system, and an expressed protein band of approximately 29 kDa was detected in membrane fractions using SDS polyacrylamide gel electrophoresis.

Clone CJ145_{—}1”

A polynucleotide of the present invention has been identified as clone “CJ145_{—}1”.

CJ145_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CJ145_{—}1 is a full-length clone, including the entire coding sequence of a secreted protein (also referred to herein as “CJ145_{—}1 protein”).

The nucleotide sequence of CJ145_{—}1 as presently determined is reported in SEQ ID NO:160, and includes a poly(A) tail. What applicants presently believe to be the proper reading frame and the predicted amino acid sequence of the CJ145_{—}1 protein corresponding to the foregoing nucleotide sequence is reported in SEQ ID NO:161. Amino acids 6 to 18 of SEQ ID NO:161 are a predicted leader/signal sequence, with the predicted mature amino acid sequence beginning at amino acid 19. Due to the hydrophobic nature of the predicted leader/signal sequence, it is likely to act as a transmembrane domain should the predicted leader/signal sequence not be separated from the remainder of the CJ145_{—}1 protein.

The EcoRI/NotI restriction fragment obtainable from the deposit containing clone CJ45_{—}1 should be approximately 3600 bp.

The nucleotide sequence disclosed herein for CJ145_{—}1 was searched against the GenBank and GeneSeq nucleotide sequence databases using BLASTN/BLASTX and FASTA search protocols. CJ145_{—}1 demonstrated at least some similarity with sequences identified as R43655 (yc86b04.s1 Homo sapiens cDNA clone 22829 3′), R50995 (yg63f06.s1 Homo sapiens cDNA clone 37377 3′ similar to contains MER22 repetitive element), and W92748 (zd92h03.s1 Soares fetal heart NbHH19W Homo sapiens cDNA clone 356981 3′). Based upon sequence similarity, CJ145_{—}1 proteins and each similar protein or peptide may share at least some activity. The nucleotide sequence of CJ145_{—}1 indicates that it may contain a CA simple repeat element.

Clone “CJ160_{—}11”

A polynucleotide of the present invention has been identified as clone “CJ160_{—}11”. CJ160_{—}11 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CJ160_{—}11 is a full-length clone, including the entire coding sequence of a secreted protein (also referred to herein as “CJ160_{—}11 protein”).

The nucleotide sequence of CJ160_{—}11 as presently determined is reported in SEQ ID NO:162, and includes a poly(A) tail. What applicants presently believe to be the proper reading frame and the predicted amino acid sequence of the CJ160_{—}11 protein corresponding to the foregoing nucleotide sequence is reported in SEQ ID NO:163. Amino adds 17 to 29 of SEQ ID NO:163 are a predicted leader/signal sequence, with the predicted mature amino acid sequence beginning at amino acid 30. Due to the hydrophobic nature of the predicted leader/signal sequence, it is likely to act as a transmembrane domain should the predicted leader/signal sequence not be separated from the remainder of the CJ160_{—}11 protein.

The EcoRI/NotI restriction fragment obtainable from the deposit containing clone CJ160_{—}11 should be approximately 1700 bp. The nucleotide sequence disclosed herein for CJ160_{—}11 was searched against the GenBank and GeneSeq nucleotide sequence databases using BLASTN/BLASTX and FASTA search protocols. CJ160_{—}11 demonstrated at least some similarity with sequences identified as AA024511 (ze76e04.s1 Soares fetal heart NbHH19W Homo sapiens cDNA clone 364926 3′) and AC000074 (00884; HTGS phase 3, complete sequence). Based upon sequence similarity, CJ160 11 proteins and each similar protein or peptide may share at least some activity.

CJ160_{—}11 protein was expressed in a COS cell expression system, and an expressed protein band of approximately 96 kDa was detected in conditioned medium using SDS polyacrylamide gel electrophoresis.

Clone “CO20_{—}1”

A polynucleotide of the present invention has been identified as clone “CO20_{—}1”. CO20_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CO20_{—}1 is a full-length done, including the entire coding sequence of a secreted protein (also referred to herein as “CO20_{—}1 protein”).

The nucleotide sequence of the 5′ portion of CO20_{—}1 as presently determined is reported in SEQ ID NO:164. What applicants presently believe is the proper reading frame for the coding region is indicated in SEQ ID NO:165. The predicted amino acid sequence of the CO20_{—}1 protein corresponding to the foregoing nucleotide sequence is reported in SEQ ID NO:165. Amino acids 17 to 29 of SEQ ID NO:165 are a predicted leader/signal sequence, with the predicted mature amino acid sequence beginning at amino acid 30. Due to the hydrophobic nature of the predicted leader/signal sequence, it is likely to act as a transmembrane domain should the predicted leader/signal sequence not be separated from the remainder of the CO20_{—}1 protein. Additional nucleotide sequence from the 3′ portion of CO20_{—}1, including a poly(A) tail, is reported in SEQ ID NO:166.

The EcoRI/NotI restriction fragment obtainable from the deposit containing clone CO20_{—}1 should be approximately 2400 bp.

The nucleotide sequence disclosed herein for CO20_{—}1 was searched against the GenBank and GeneSeq nucleotide sequence databases using BLASTN/BLASTX and FASTA search protocols. CO20_{—}1 demonstrated at least some similarity with sequences identified as AA045770 (zl68b10.s1 Stratagene colon (#937204) Homo sapiens cDNA clone 509755 3′ similar to SW:R13A_HUMAN P40429 60S RIBOSOMAL PROTEIN L13A), AA070899 (zm66c01.s1 Stratagene neuroepithelium (#937231) Homo sapiens cDNA clone 530592 3′ similar to contains Alu repetitive element), AA325205 (EST28155 Cerebellum II Homo sapiens cDNA 5′ end), N22253 (yw36a08.s1 Homo sapiens cDNA clone 254294 3′ similar to SP S29539 S29539 BASIC PROTEIN, 23K), R01933 (ye85g07.s1 Homo sapiens cDNA clone 124572 3′ similar to SP:S29539 S29539 BASIC PROTEIN, 0.23K), R12008 (yf51f04.r1 Homo sapiens cDNA clone 25456 5′), R39848 (yf51f04.s1 Homo sapiens cDNA clone 25456 3′ similar to contains Alu repetitive element; contains PTR5 repetitive element), R56565 (yg91c12.r1 Homo sapiens cDNA clone 40891 5′), T19487 (Human gene signature HUMGS00543), T30988 (ES25695 Homo sapiens cDNA 5′ end similar to None), U37026 (Rattus norvegicus brain sodium channel beta 2 subunit (SCNB2) mRNA, complete cds), and X56932 (H. sapiens mRNA for 23 kD highly basic protein). The predicted amino acid sequence disclosed herein for CO20_{—}1 was searched against the GenPept and GeneSeq amino acid sequence databases using the BLASTX search protocol. The predicted CO20_{—}1 protein demonstrated at least some similarity to sequences identified as U37026 (sodium channel beta 2 subunit [Rattus norvegicus]), U58658 (unknown [Homo sapiens]); and X56932 (23 kD highly basic protein [Homo sapiens]). The sodium channel beta 2 subunit is a glycoprotein with an extracellular domain containing an immunoglobulin-like fold with similarity to the neural cell adhesion molecule contactin. Based upon sequence similarity, CO20_{—}1 proteins and each similar protein or peptide may share at least some activity. The nucleotide sequence of CO20_{—}1 indicates that it may contain an Alu repetitive element.

Clone “CO223_{—}3”

A polynucleotide of the present invention has been identified as clone “CO223_{—}3”.

CO223_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CO223_{—}3 is a full-length done, including the entire coding sequence of a secreted protein (also referred to herein as “CO223_{—}3 protein”). The nucleotide sequence of CO223_{—}3 as presently determined is reported in SEQ ID NO:167, and includes a poly(A) tail. What applicants presently believe to be the proper reading frame and the predicted amino acid sequence of the CO223_{—}3 protein corresponding to the foregoing nucleotide sequence is reported in SEQ ID NO:168. Amino acids 35 to 47 of SEQ ID NO:168 are a predicted leader/signal sequence, with the predicted mature amino acid sequence beginning at amino acid 48. Due to the hydrophobic nature of the predicted leader/signal sequence, it is likely to act as a transmembrane domain should the predicted leader/signal sequence not be separated from the remainder of the CO223_{—}3 protein.

The EcoRI/NotI restriction fragment obtainable from the deposit containing clone CO223_{—}3 should be approximately 700 bp.

The nucleotide sequence disclosed herein for CO223_{—}3 was searched against the GenBank and GeneSeq nucleotide sequence databases using BLASTN/BLASTX and FASTA search protocols. CO223_{—}3 demonstrated at least some similarity with sequences identified as AA004498 (zh87b06.r1 Soares fetal liver spleen INFLS S1 Homo sapiens cDNA clone 428243 5′ similar to gb M62505 C5A ANAPHYLATOXIN CHEMOTACTIC RECEPTOR (HUMAN);contains L1.t1 L1 repetitive element) and U47924 (Human chromosome 12p13 gene cluster, surface antigen CD4 (CD4), A, B, G-protein beta-3 subunit (GNB3), isopeptidase T (ISOT) and triosephosphate isomerase (TPI) genes, complete cds). Based upon sequence similarity, CO223_{—}3 proteins and each similar protein or peptide may share at least some activity.

The 3′ end of the CO223_{—}3 polynucleotide sequence contains a 54-bp sequence that is repeated three times in the clone; these repeats begin at positions 314, 368, and 422 of SEQ ID NO:167 and encode amino acids 47 to 64,65 to 82, and 83 to 99 of SEQ ID NO:168, respectively.

Clone “CO310_{—}2”

A polynucleotide of the present invention has been identified as clone “CO310_{—}2”. CO310_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CO310_{—}2 is a full-length clone, including the entire coding sequence of a secreted protein (also referred to herein as “CO310_{—}2 protein”).

The nucleotide sequence of CO310_{—}2 as presently determined is reported in SEQ ID NO:169, and includes a poly(A) tail. What applicants presently believe to be the proper reading frame and the predicted amino acid sequence of the CO310_{—}2 protein corresponding to the foregoing nucleotide sequence is reported in SEQ ID NO:170.

The EcoRI/NotI restriction fragment obtainable from the deposit containing clone CO310_{—}2 should be approximately 1400 bp.

The nucleotide sequence disclosed herein for CO310_{—}2 was searched against the GenBank and GeneSeq nucleotide sequence databases using BLASTN/BLASTX and FASTA search protocols. No hits were found in the database. The nucleotide sequence of CO310_{—}2 indicates that it may contain an L1 repetitive element

Clone “CP258 3”

A polynucleotide of the present invention has been identified as clone “CP258_{—}3”. CP258_{—}3 was isolated from a human adult salivary gland cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CP258_{—}3 is a full-length done, including the entire coding sequence of a secreted protein (also referred to herein as “CP258_{—}3 protein”).

The nucleotide sequence of CP258_{—}3 as presently determined is reported in SEQ ID NO:171, and includes a poly(A) tail. What applicants presently believe to be the proper reading frame and the predicted amino acid sequence of the CP258_{—}3 protein corresponding to the foregoing nucleotide sequence is reported in SEQ ID NO:172. Amino acids 3 to 15 of SEQ ID NO:172 are a predicted leader/signal sequence, with the predicted mature amino acid sequence beginning at amino acid 16. Due to the hydrophobic nature of the predicted leader/signal sequence, it is likely to act as a transmembrane domain should the predicted leader/signal sequence not be separated from the remainder of the CP258_{—}3 protein.

The EcoRI/NotI restriction fragment obtainable from the deposit containing clone CP258_{—}3 should be approximately 560 bp.

The nucleotide sequence disclosed herein for CP258_{—}3 was searched against the GenBank and GeneSeq nucleotide sequence databases using BLASTN/BLASTX and FASTA search protocols. No hits were found in the database.

CP258_{—}3 protein was expressed in a COS cell expression system, and an expressed protein band of approximately 26 kDa was detected in conditioned medium and membrane fractions using SDS polyacrylamide gel electrophoresis.

Clone “CW1155_{—}3”

A polynucleotide of the present invention has been identified as clone “CW1155_{—}3”. CW1155_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CW1155_{—}3 is a full-length done, including the entire coding sequence of a secreted protein (also referred to herein as “CW1155_{—}3 protein”).

The nucleotide sequence of CW1155_{—}3 as presently determined is reported in SEQ ID NO 173, and includes a poly(A) tail. What applicants presently believe to be the proper reading frame and the predicted amino acid sequence of the CW1155_{—}3 protein corresponding to the foregoing nucleotide sequence is reported in SEQ ID NO:174. Amino acids 220 to 232 of SEQ ID NO:174 are a predicted leader/signal sequence, with the predicted mature amino acid sequence beginning at amino acid 233. Due to the hydrophobic nature of the predicted leader/signal sequence, it is likely to act as a transmembrane domain should the predicted leader/signal sequence not be separated from the remainder of the CW1155_{—}3 protein.

The EcoRI/NotI restriction fragment obtainable from the deposit containing clone CW1155_{—}3 should be approximately 1170 bp. The nucleotide sequence disclosed herein for CW1155_{—}3 was searched against the GenBank and GeneSeq nucleotide sequence databases using BLASTN/BLASTX and FASTA search protocols. CW1155_{—}3 demonstrated at least some similarity with sequences identified as AA169043 (ms36h08.r1 Stratagene mouse heart (#937316) Mus musculus cDNA clone 613695 5′), D86145 (Rat mRNA), and H29261 (ym32b03.s1 Homo sapiens cDNA clone 49733 3′). Based upon sequence similarity, CW1155_{—}3 proteins and each similar protein or peptide may share at least some activity.

Clone “CZ247_{—}2”

A polynucleotide of the present invention has been identified as clone “CZ247_{—}2”. CZ247_{—}2 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CZ247_{—}2 is a full-length done, including the entire coding sequence of a secreted protein (also referred to herein as “CZ247_{—}2 protein”).

The nucleotide sequence of CZ247_{—}2 as presently determined is reported in SEQ ID NO:175, and includes a poly(A) tail. What applicants presently believe to be the proper reading frame and the predicted amino acid sequence of the CZ247_{—}2 protein corresponding to the foregoing nucleotide sequence is reported in SEQ ID NO:176. Amino acids 545 to 557 of SEQ ID NO:176 are a predicted leader/signal sequence, with the predicted mature amino acid sequence beginning at amino acid 558. Due to the hydrophobic nature of the predicted leader/signal sequence, it is likely to act as a transmembrane domain should the predicted leader/signal sequence not be separated from the remainder of the C247_{—}2 protein.

The EcoRI/NotI restriction fragment obtainable from the deposit containing clone CZ247_{—}2 should be approximately 2300 bp.

The nucleotide sequence disclosed herein for CZ247_{—}2 was searched against the GenBank and GeneSeq nucleotide sequence databases using BLASTN/BLASTX and FASTA search protocols. CZ247_{—}2 demonstrated at least some similarity with sequences identified as T09256 (Human ara Kb beta-galactosidase fusion protein coding sequence), W27222 (26h9 Human retina cDNA randomly primed sublibrary Homo sapiens cDNA), and W72736 (zd7le02.s1 Soares fetal heart NbHH19W Homo sapiens cDNA clone 346106 3′). The predicted amino acid sequence disclosed herein for CZ247_{—}2 was searched against the GenPept and GeneSeq amino acid sequence databases using the BLASTX search protocol. The predicted CZ247_{—}2 protein demonstrated at least some similarity to sequences identified as R88069 (Human ara Kb beta-galactosidase fusion protein). Based upon sequence similarity, CZ247_{—}2 proteins and each similar protein or peptide may share at least some activity.

Clone “AM666_{—}1”

A polynucleotide of the present invention has been identified as clone “AM666_{—}1”.

AM666_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AM666_{—}1 is a full-length clone, including the entire coding sequence of a secreted protein (also referred to herein as “AM666_{—}1 protein”).

The nucleotide sequence of AM666_{—}1 as presently determined is reported in SEQ ID NO:177, and includes a poly(A) tail. What applicants presently believe to be the proper reading frame and the predicted amino acid sequence of the AM666_{—}1 protein corresponding to the foregoing nucleotide sequence is reported in SEQ ID NO:178. Amino acids 15 to 27 of SEQ ID NO:178 are a predicted leader/signal sequence, with the predicted mature amino acid sequence beginning at amino acid 28. Due to the hydrophobic nature of the predicted leader/signal sequence, it is likely to act as a transmembrane domain should the predicted leader/signal sequence not be separated from the remainder of the AM666_{—}1 protein.

The EcoRI/NotI restriction fragment obtainable from the deposit containing clone AM666_{—}1 should be approximately 1300 bp. The nucleotide sequence disclosed herein for AM666_{—}1 was searched against the GenBank and GeneSeq nucleotide sequence databases using BLASTN/BLASTX and FASTA search protocols. AM666_{—}1 demonstrated at least some similarity with sequences identified as AA493985 (nh07g08.s1 NCI_CGAP_Thy1 Homo sapiens cDNA clone). Based upon sequence similarity, AM666_{—}1 proteins and each similar protein or peptide may share at least some activity.

AM666_{—}1 protein was expressed in a COS cell expression system, and an expressed protein band of approximately 17 kDa was detected in membrane fractions using SDS polyacrylamide gel electrophoresis.

Clone “BN387_{—}3”

A polynucleotide of the present invention has been identified as clone “BN387_{—}3”. BN387_{—}3 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BN387_{—}3 is a full-length clone, including the entire coding sequence of a secreted protein (also referred to herein as “BN387_{—}3 protein”). The nucleotide sequence of BN387_{—}3 as presently determined is reported in SEQ ID NO:179, and includes a poly(A) tail. What applicants presently believe to be the proper reading frame and the predicted amino acid sequence of the BN387_{—}3 protein corresponding to the foregoing nucleotide sequence is reported in SEQ ID NO:180. Amino acids 14 to 26 of SEQ ID NO:180 are a predicted leader/signal sequence, with the predicted mature amino acid sequence beginning at amino acid 27. Due to the hydrophobic nature of the predicted leader/signal sequence, it is likely to act as a transmembrane domain should the predicted leader/signal sequence not be separated from the remainder of the BN387_{—}3 protein.

The EcoRI/NotI restriction fragment obtainable from the deposit containing clone BN387_{—}3 should be approximately 2000 bp.

The nucleotide sequence disclosed herein for BN387_{—}3 was searched against the GenBank and GeneSeq nucleotide sequence databases using BLASTN/BLASTX and FASTA search protocols. BN387_{—}3 demonstrated at least some similarity with sequences identified as H16912 (ym39d01.r1 Homo sapiens cDNA clone 50771 5′). Based upon sequence similarity, BN387_{—}3 proteins and each similar protein or peptide may share at least some activity.

BN387_{—}3 protein was expressed in a COS cell expression system, and an expressed protein band of approximately 30 kDa was detected in conditioned medium using SDS polyacrylamide gel electrophoresis.

Clone “B0135_{—}2”

A polynucleotide of the present invention has been identified as clone “BQ135_{—}2”. BQ135_{—}2 was isolated from a human adult colon (adenocarcinoma Caco2) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BQ135_{—}2 is a full-length clone, including the entire coding sequence of a secreted protein (also referred to herein as “BQ135 2 protein”). The nucleotide sequence of BQ135 2 as presently determined is reported in SEQ ID NO:181, and includes a poly(A) tail. What applicants presently believe to be the proper reading frame and the predicted amino acid sequence of the BQ135_{—}2 protein corresponding to the foregoing nucleotide sequence is reported in SEQ ID NO:182.

The EcoRI/NotI restriction fragment obtainable from the deposit containing clone BQ135_{—}2 should be approximately 1200 bp.

The nucleotide sequence disclosed herein for BQ135_{—}2 was searched against the GenBank and GeneSeq nucleotide sequence databases using BLASTN/BLASTX and FASTA search protocols. BQ135_{—}2 demonstrated at least some similarity with sequences identified as AA023751 (mh81f01.r1 Soares mouse placenta 4NbMP13.5 14.5 Mus musculus cDNA clone 457369 5′), AA105433 (ml83g01.r1 Stratagene mouse kidney (#937315) Mus musculus cDNA clone 518640 5′), D64061 (Rat brain mRNA for annexin V-binding protein (ABP-7), partial cds), and N67257 (yz49b08.s1 Homo sapiens cDNA clone 286359 3′). The predicted amino acid sequence disclosed herein for BQ135_{—}2 was searched against the GenPept and GeneSeq amino acid sequence databases using the BLASTX search protocol. The predicted BQ135_{—}2 protein demonstrated at least some similarity to sequences identified as D64061 (annexin V-binding protein (ABP-7) [Rattus norvegicus]). Annexins associate with membranes and act as ion channels, they can also act as an autocrine factor that enhances osteoclast formation and bone resorption. Annexins have been localized in nucleoli and mitochondria but also in the cytoplasm, plasma (i.e. blood) and in association with vesicles. They are probably involved in fusing vesicles to each other and to plasma membranes causing secretion of vesicular contents. Specifically they have a calcium-dependent ability to bind phospholipids. Thus they are membrane associated. It is possible that annexin-binding proteins are also membrane associated even though they are highly hydrophilic through the same mechanism (electrostatic interaction with phospholipids of membranes). Based upon sequence similarity, BQ135_{—}2 proteins and each similar protein or peptide may share at least some activity.

Clone “CR678_{—}1”

A polynucleotide of the present invention has been identified as clone “CR678_{—}1”. CR678_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CR678_{—}1 is a full-length clone, including the entire coding sequence of a secreted protein (also referred to herein as “CR6781 protein”).

The nucleotide sequence of CR678_{—}1 as presently determined is reported in SEQ ID NO:183, and includes a poly(A) tail. What applicants presently believe to be the proper reading frame and the predicted amino acid sequence of the CR678_{—}1 protein corresponding to the foregoing nucleotide sequence is reported in SEQ ID NO:184.

The EcoRI/NotI restriction fragment obtainable from the deposit containing clone CR678_{—}1 should be approximately 870 bp.

The nucleotide sequence disclosed herein for CR678_{—}1 was searched against the GenBank and GeneSeq nucleotide sequence databases using BLASTN/BLASTX and FASTA search protocols. CR678_{—}1 demonstrated at least some similarity with sequences identified as X85232 (H. sapiens chromosome 3 sequences). Based upon sequence similarity, CR678_{—}1 proteins and each similar protein or peptide may share at least some activity. The nucleotide sequence of CR678_{—}1 indicates that it may contain an Alu repetitive element.

Clone “CW420_{—}2”

A polynucleotide of the present invention has been identified as clone “CW420_{—}2”. CW420_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CW420_{—}2 is a full-length clone, including the entire coding sequence of a secreted protein (also referred to herein as “CW420_{—}2 protein”).

The nucleotide sequence of CW420_{—}2 as presently determined is reported in SEQ ID NO:185, and includes a poly(A) tail. What applicants presently believe to be the proper reading frame and the predicted amino acid sequence of the CW420_{—}2 protein corresponding to the foregoing nucleotide sequence is reported in SEQ ID NO:186.

The EcoRI/NotI restriction fragment obtainable from the deposit containing clone CW420_{—}2 should be approximately 5100 bp.

The nucleotide sequence disclosed herein for CW420_{—}2 was searched against the GenBank and GeneSeq nucleotide sequence databases using BLASTN/BLASTX and FASTA search protocols. CW420_{—}2 demonstrated at least some similarity with sequences identified as T55440 (yb38eO9.s1 Homo sapiens cDNA clone 73480 3′). Based upon sequence similarity, CW420_{—}2 proteins and each similar protein or peptide may share at least some activity. The TopPredII computer program predicts two potential transmembrane domains within the CW420_{—}2 protein sequence centered around amino acids 500 and 1270 of SEQ ID NO:186, respetively.

Clone “CW795_{—}2”

A polynucleotide of the present invention has been identified as clone “CW795_{—}2”. CW795_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CW795_{—}2 is a full-length clone, including the entire coding sequence of a secreted protein (also referred to herein as “CW795_{—}2 protein”).

The nucleotide sequence of CW795_{—}2 as presently determined is reported in SEQ ID NO:187, and includes a poly(A) tail. What applicants presently believe to be the proper reading frame and the predicted amino acid sequence of the CW795_{—}2 protein corresponding to the foregoing nucleotide sequence is reported in SEQ ID NO:188.

The EcoRI/NotI restriction fragment obtainable from the deposit containing clone CW795_{—}2 should be approximately 3000 bp.

The nucleotide sequence disclosed herein for CW795_{—}2 was searched against the GenBank and GeneSeq nucleotide sequence databases using BLASTN/BLASTX and FASTA search protocols. CW795_{—}2 demonstrated at least some similarity with sequences identified as AA115676 (zl86a09,s1 Stratagene colon (#937204) Homo sapiens cDNA clone 511480 3′), N22955 (yw44h07.s1 Homo sapiens cDNA clone 255133 3′), and W56804 (zd16g06.s1 Soares fetal heart NbHH19W Homo sapiens cDNA clone 340858 3′). The predicted amino acid sequence disclosed herein for CW795_{—}2 was searched against the GenPept, GeneSeq, and SwissProt amino acid sequence databases using the BLASTX search protocol. The predicted CW795_{—}2 protein demonstrated at least some similarity to sequences identified as X81068 (probable mitochondrial protein) and the yeast proteins real and afg3 (tat-binding homologues). Based upon sequence similarity, CW795_{—}2 proteins and each similar protein or peptide may share at least some activity. The TopPredII computer program predicts two potential transmembrane domains within the CW795_{—}2 protein sequence centered around amino acids 60 and 170 of SEQ ID NO:188, respectively.

CW795_{—}2 protein was expressed in a COS cell expression system, and an expressed protein band of approximately 10 kDa was detected in membrane fractions using SDS polyacrylamide gel electrophoresis.

Clone “CW823_{—}3”

A polynucleotide of the present invention has been identified as clone “CW823_{—}3”. CW823_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CW823_{—}3 is a full-length clone, including the entire coding sequence of a secreted protein (also referred to herein as “CW823_{—}3 protein”).

The nucleotide sequence of CW823_{—}3 as presently determined is reported in SEQ ID NO:189, and includes a poly(A) tail. What applicants presently believe to be the proper reading frame and the predicted amino acid sequence of the CW823_{—}3 protein corresponding to the foregoing nucleotide sequence is reported in SEQ ID NO:190.

The EcoRI/NotI restriction fragment obtainable from the deposit containing clone CW823_{—}3 should be approximately 600 bp.

The nucleotide sequence disclosed herein for CW823_{—}3 was searched against the GenBank and GeneSeq nucleotide sequence databases using BLASTN/BLASTX and FASTA search protocols. No hits were found in the database.

Clone “DF989 3”

A polynucleotide of the present invention has been identified as clone “DF989_{—}3”. DF989_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. DF989_{—}3 is a full-length done, including the entire coding sequence of a secreted protein (also referred to herein as “DF989_{—}3 protein”).

The nucleotide sequence of the 5′ portion of DF989_{—}3 as presently determined is reported in SEQ ID NO:191. What applicants presently believe is the proper reading frame for the coding region is indicated in SEQ ID NO:192. The predicted amino acid sequence of the DF989_{—}3 protein corresponding to the foregoing nucleotide sequence is reported in SEQ ID NO:192. Amino acids 2 to 14 of SEQ ID NO:192 are a predicted leader/signal sequence, with the predicted mature amino acid sequence beginning at amino acid 15. Due to the hydrophobic nature of the predicted leader/signal sequence, it is likely to act as a transmembrane domain should the predicted leader/signal sequence not be separated from the remainder of the DF989_{—}3 protein. Additional nucleotide sequence from the 3′ portion of DF989 3, including a poly(A) tail, is reported in SEQ ID NO:193.

The EcoRI/NotI restriction fragment obtainable from the deposit containing clone DF989_{—}3 should be approximately 1800 bp.

The nucleotide sequence disclosed herein for DF989_{—}3 was searched against the GenBank and GeneSeq nucleotide sequence databases using BLASTN/BLASTX and FASTA search protocols. DF989_{—}3 demonstrated at least some similarity with sequences identified as R24724 (yg43c05.r1 Homo sapiens cDNA clone 35337 5′) and T33717 (EST58870 Homo sapiens cDNA 5′ end similar to None). Based upon sequence similarity, DF989_{—}3 proteins and each similar protein or peptide may share at least some activity.

Clone “DL162_{—1”}

A polynucleotide of the present invention has been identified as clone “DL162_{—}1”. DL162_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. DL162_{—}1 is a full-length clone, including the entire coding sequence of a secreted protein (also referred to herein as “DL162_{—}1 protein”).

The nucleotide sequence of DL162_{—}1 as presently determined is reported in SEQ ID NO:194, and includes a poly(A) tail. What applicants presently believe to be the proper reading frame and the predicted amino acid sequence of the DL162_{—}1 protein corresponding to the foregoing nucleotide sequence is reported in SEQ ID NO:195. Amino acids 28 to 40 of SEQ ID NO:195 are a predicted leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the DL162_{—}1 protein.

The EcoRI/NotI restriction fragment obtainable from the deposit containing clone DL162_{—}1 should be approximately 875 bp.

The nucleotide sequence disclosed herein for DL162_{—}1 was searched against the GenBank and GeneSeq nucleotide sequence databases using BLASTN/BLASTX and FASTA search protocols. No hits were found in the database.

Clone “DL162_{—}2”

A polynucleotide of the present invention has been identified as clone “DL162_{—}2”. DL162_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. DL162_{—}2 is a full-length clone, including the entire coding sequence of a secreted protein (also referred to herein as “DL162_{—}2 protein”).

The nucleotide sequence of DL162_{—}2 as presently determined is reported in SEQ ID NO:196, and includes a poly(A) tail. What applicants presently believe to be the proper reading frame and the predicted amino acid sequence of the DL162_{—}2 protein corresponding to the foregoing nucleotide sequence is reported in SEQ ID NO:197. Amino acids 1 to 13 of SEQ ID NO:197 are a predicted leader/signal sequence, with the predicted mature amino acid sequence beginning at amino acid 14. Due to the hydrophobic nature of the predicted leader/signal sequence, it is likely to act as a transmembrane domain should the predicted leader/signal sequence not be separated from the remainder of the DL162_{—}2 protein.

The EcoRI/NotI restriction fragment obtainable from the deposit containing clone DL162_{—}2 should be approximately 4000 bp.

The predicted amino acid sequence disclosed herein for DL162_{—}2 was searched against the GenPept and GeneSeq amino acid sequence databases using the BLASTX search protocol. The predicted DL162_{—}2 protein demonstrated at least some similarity to sequences identified as AB002309 (KIAAA0311 protein [Homo sapiens]). The TopPredII computer program predicts a potential transmembrane domains within the DL162_{—}2 protein sequence near the carboxyl terminus of SEQ ID NO:197.

DL162_{—}2 protein was expressed in a COS cell expression system, and an expressed protein band of approximately 160 kDa was detected in membrane fractions using SDS polyacrylamide gel electrophoresis.

Clone “EC172_{—}1”

A polynucleotide of the present invention has been identified as clone “EC172_{—}1”. EC172_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. EC172_{—}1 is a full-length clone, including the entire coding sequence of a secreted protein (also referred to herein as “EC172_{—}1 protein”).

The nucleotide sequence of EC172_{—}1 as presently determined is reported in SEQ ID NO:198, and includes a poly(A) tail. What applicants presently believe to be the proper reading frame and the predicted amino acid sequence of the EC172_{—}1 protein corresponding to the foregoing nucleotide sequence is reported in SEQ ID NO:199. Amino acids 659 to 671 of SEQ ID NO:199 are a predicted leader/signal sequence, with the predicted mature amino acid sequence beginning at amino acid 672. Due to the hydrophobic nature of the predicted leader/signal sequence, it is likely to act as a transmembrane domain should the predicted leader/signal sequence not be separated from the remainder of the EC172_{—}1 protein.

The EcoRI/NotI restriction fragment obtainable from the deposit containing clone EC172_{—}1 should be approximately 4000 bp.

The nucleotide sequence disclosed herein for EC172_{—}1 was searched against the GenBank and GeneSeq nucleotide sequence databases using BLASTN/BLASTX and FASTA search protocols. EC172_{—}1 demonstrated at least some similarity with sequences identified as H31192 (EST104991 Rattus sp. cDNA 3′ end similar to C. elegans hypothetical protein ZK1098.10) and U29585 (Streptococcus pyogenes emm18.1). The predicted amino acid sequence disclosed herein for EC172_{—}1 was searched against the GenPept and GeneSeq amino acid sequence databases using the BLASTX search protocol. The predicted EC172_{—}1 protein demonstrated at least some similarity to sequences identified as Z22176 (ZK1098.10 [Caenorhabditis elegans]). Based upon sequence similarity, EC172_{—}1 proteins and each similar protein or peptide may share at least some activity.

Deposit of Clones

Clones AX65_{—}22, BD335_{—}14, BG241_{—}1, BL187_{—}4, BL249_{—}18, BO71_{—}1, BO365_{—}2, BV51_{—}1, BV140_{—}3, BV141_{—}2, CC194_{—}4, and DA136_{—}11 were deposited on Oct. 3, 1996 with the ATCC (American Type Culture Collection, 10801 University Boulevard, Manassas, Va. 20110-2209 U.S.A.) as an original deposit under the Budapest Treaty and were given the accession number 98196, from which each clone comprising a particular polynucleotide is obtainable.

Clones AR415_{—}4, AS63_{—}29, BG160_{—}1, BO432_{—}4, BO538_{—}2, BR595_{—}4, CI490_{—}2, CI522_{—}1, CN238_{—}1, CO390_{—}1, and AY304_{—}1 (an additional isolate of clone AY304_{—}14) were deposited on Oct. 25, 1996 with the ATCC (American Type Culture Collection, 10801 University Boulevard, Manassas, Va. 20110-2209 U.S.A.) as an original deposit under the Budapest Treaty and were given the accession number 98232, from which each clone comprising a particular polynucleotide is obtainable. Clone AY304_{—}14 was deposited on Oct. 23, 1997 with the ATCC (American Type Culture Collection, 10801 University Boulevard, Manassas, Va. 20110-2209 U.S.A.) as an original deposit under the Budapest Treaty and was given the accession number 98561.

Clones AJ20_{—}2, AR440_{—}1, AS164_{—}1, AX8_{—}1, BD176_{—}3, BD339_{—}1, BD427_{—}1, BL229_{—}22, BV123_{—}16, and CH377_{—}1 were deposited on Nov. 15, 1996 with the ATCC (American Type Culture Collection, 10801 University Boulevard, Manassas, Va. 20110-2209 U.S.A.) as an original deposit under the Budapest Treaty and were given the accession number 98261, from which each clone comprising a particular polynucleotide is obtainable.

Clones BD441_{—}1, BD441_{—}2, BG102_{—}3, BK158_{—}1, BP163_{—}1, BZ16_{—}3, CC182_{—}1, CG109_{—}1 and CJ397_{—}1 were deposited on Nov. 20, 1996 with the ATCC (American Type Culture Collection, 10801 University Boulevard, Manassas, Va. 20110-2209 U.S.A.) as an original deposit under the Budapest Treaty and were given the accession number 98264, from which each clone comprising a particular polynucleotide is obtainable.

Clones AM795_{—}4, AT340_{—}1, BG132_{—}1, BG219_{—}2, BG366_{—}2, BV172_{—}2, CC247_{—}10, CI480_{—}9, CO722_{—}1, and CT748_{—}2 were deposited on Dec. 5, 1996 with the ATCC (American Type Culture Collection, 10801 University Boulevard, Manassas, Va. 20110-2209 U.S.A.) as an original deposit under the Budapest Treaty and were given the accession number 98271, from which each clone comprising a particular polynucleotide is obtainable.

Clones AJ1_{—}1, AQ73_{—}3, BG142_{—}1, BV66_{—}1, BV291_{—}3, CK201_{—}1, CQ331_{—}2, CT550_{—}1, CT585_{—}1 and CT797_{—}3 were deposited on Dec. 13, 1996 with the ATCC (American Type Culture Collection, 10801 University Boulevard, Manassas, Va. 20110-2209 U.S.A.) as an original deposit under the Budapest Treaty and were given the accession number 98278, from which each clone comprising a particular polynucleotide is obtainable.

Clones CB107_{—}1, CG300_{—}3, CJ145_{—}1, CJ160_{—}11, CO20_{—}1, CO223_{—}1, CO310_{—}2, CP258_{—}3, CW1155_{—}3 and CZ247_{—}2 were deposited on Dec. 17, 1996 with the ATCC (American Type Culture Collection, 10801 University Boulevard, Manassas, Va. 20110-2209 U.S.A.) as an original deposit under the Budapest Treaty and were given the accession number 98279, from which each clone comprising a particular polynucleotide is obtainable. Clone CO223_{—}3 was deposited on Jan. 9, 1997 with the ATCC (American Type Culture Collection, 10801 University Boulevard, Manassas, Va. 20110-2209 U.S.A.) as an original deposit under the Budapest Treaty and was given the accession number 98291.

Clones AM666_{—}1, BN387_{—}3, BQ135_{—}2, CR678_{—}1, CW420_{—}2, CW795_{—}2, CW823_{—}3, DF989_{—}3, DL162_{—}2, DL162_{—}1, and EC172_{—}1 were deposited on Jan. 10, 1997 with the ATCC (American Type Culture Collection, 10801 University Boulevard, Manassas, Va. 20110-2209 U.S.A.) as an original deposit under the Budapest Treaty and were given the accession number 98292, from which each clone comprising a particular polynucleotide is obtainable. All restrictions on the availability to the public of the deposited material will be irrevocably removed upon the granting of the patent, except for the requirements specified in 37 C.F.R. § 1.808(b), and the term of the deposit will comply with 37 C.F.R. § 1.806.

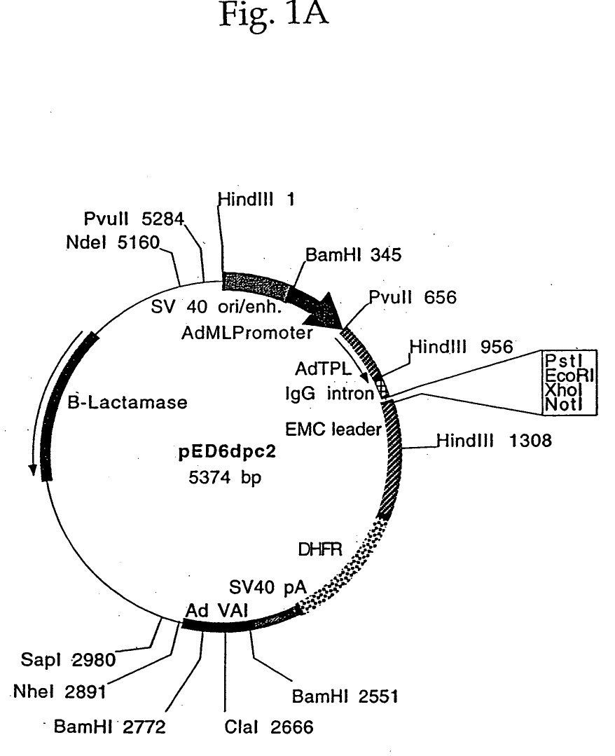

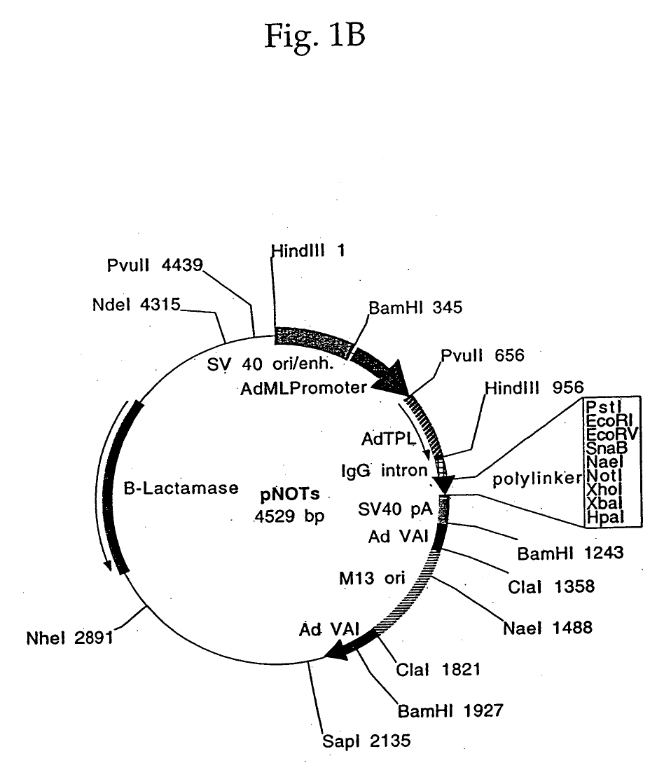

Each clone has been transfected into separate bacterial cells (E. coli) in this composite deposit. Each clone can be removed from the vector in which it was deposited by performing an EcoRI/NotI digestion (5′ site, EcoRI; 3′ site, NotI) to produce the appropriate fragment for such clone. Each clone was deposited in either the pED6 or pNOTs vector depicted in FIGS. 1A and 1B, respectively. The pED6dpc2 vector (“pED6”) was derived from pED6dpc1 by insertion of a new polylinker to facilitate cDNA cloning (Kaufman et al., 1991, Nucleic Acids Res. 19: 4485-4490); the pNOTs vector was derived from pMT2 (Kaufman et al., 1989, Mol. Cell. Biol. 9: 946-958) by deletion of the DHFR sequences, insertion of a new polylinker, and insertion of the M13 origin of replication in the ClaI site. In some instances, the deposited clone can become “flipped” (i.e., in the reverse orientation) in the deposited isolate. In such instances, the cDNA insert can still be isolated by digestion with EcoRI and NotI. However, NotI will then produce the 5′ site and EcoRI will produce the 3′ site for placement of the cDNA in proper orientation for expression in a suitable vector. The cDNA may also be expressed from the vectors in which they were deposited.

Bacterial cells containing a particular clone can be obtained from the composite deposit as follows:

An oligonucleotide probe or probes should be designed to the sequence that is known for that particular clone. This sequence can be derived from the sequences provided herein, or from a combination of those sequences. The sequence of an oligonucleotide probe that was used to isolate or to sequence each full-length clone is identified below, and should be most reliable in isolating the clone of interest.

| Clone | Probe Sequence |
|---|---|
| AX65_22 | SEQ ID NO: 200 |
| BD335_14 | SEQ ID NO: 201 |
| BG241_1 | SEQ ID NO: 202 |
| BL187_4 | SEQ ID NO: 203 |
| BL249_18 | SEQ ID NO: 204 |
| BO71_1 | SEQ ID NO: 205 |
| BO365_2 | SEQ ID NO: 206 |
| BV51_1 | SEQ ID NO: 207 |
| BV140_3 | SEQ ID NO: 208 |
| BV141_2 | SEQ ID NO: 209 |
| CC194_4 | SEQ ID NO: 210 |
| DA136_11 | SEQ ID NO: 211 |
| AR415_4 | SEQ ID NO: 212 |
| AS63_29 | SEQ ID NO: 213 |
| AY304_14 | SEQ ID NO: 214 |
| BG160_1 | SEQ ID NO: 215 |
| BO432_4 | SEQ ID NO: 216 |
| BO538_2 | SEQ ID NO: 217 |
| BR595_4 | SEQ ID NO: 218 |
| CI490_2 | SEQ ID NO: 219 |
| CI522_1 | SEQ ID NO: 220 |
| CN238_1 | SEQ ID NO: 221 |
| CO390_1 | SEQ ID NO: 222 |
| AJ20_2 | SEQ ID NO: 223 |
| AR440_1 | SEQ ID NO: 224 |
| AS164_1 | SEQ ID NO: 225 |
| AX8_1 | SEQ ID NO: 226 |
| BD176_3 | SEQ ID NO: 227 |
| BD339_1 | SEQ ID NO: 228 |
| BD427_1 | SEQ ID NO: 229 |
| BL229_22 | SEQ ID NO: 230 |
| BV123_16 | SEQ ID NO: 231 |
| CH377_1 | SEQ ID NO: 232 |
| BD441_1 | SEQ ID NO: 233 |
| BD441_2 | SEQ ID NO: 234 |
| BG102_3 | SEQ ID NO: 235 |
| BK158_1 | SEQ ID NO: 236 |
| BP163_1 | SEQ ID NO: 237 |
| BZ16_3 | SEQ ID NO: 238 |
| CC182_1 | SEQ ID NO: 239 |
| CG109_1 | SEQ ID NO: 240 |
| CJ397_1 | SEQ ID NO: 241 |
| AM795_4 | SEQ ID NO: 242 |
| AT340_1 | SEQ ID NO: 243 |
| BG132_1 | SEQ ID NO: 244 |
| BG219_2 | SEQ ID NO: 245 |
| BG366_2 | SEQ ID NO: 246 |
| BV172_2 | SEQ ID NO: 247 |
| CC247_10 | SEQ ID NO: 248 |
| CI480_9 | SEQ ID NO: 249 |
| CO722_1 | SEQ ID NO: 250 |
| CT748_2 | SEQ ID NO: 251 |
| AJ1_1 | SEQ ID NO: 252 |
| AQ73_3 | SEQ ID NO: 253 |
| BG142_1 | SEQ ID NO: 254 |
| BV66_1 | SEQ ID NO: 255 |
| BV291_3 | SEQ ID NO: 256 |
| CK201_1 | SEQ ID NO: 257 |
| CQ331_2 | SEQ ID NO: 258 |
| CT550_1 | SEQ ID NO: 259 |
| CT585_1 | SEQ ID NO: 260, SEQ ID NO: 262 |
| CT797_3 | SEQ ID NO: 261 |
| CB107_1 | SEQ ID NO: 263 |
| CG300_3 | SEQ ID NO: 264 |
| CJ145_1 | SEQ ID NO: 265 |
| CJ160_11 | SEQ ID NO: 266 |
| CO20_1 | SEQ ID NO: 267 |
| CO223_3 | SEQ ID NO: 268 |
| CO310_2 | SEQ ID NO: 269 |
| CP258_3 | SEQ ID NO: 270 |
| CW1155_3 | SEQ ID NO: 271 |
| CZ247_2 | SEQ ID NO: 272 |
| AM666_1 | SEQ ID NO: 273 |
| BN387_3 | SEQ ID NO: 274 |
| BQ135_2 | SEQ ID NO: 275 |
| CR678_1 | SEQ ID NO: 276 |
| CW420_2 | SEQ ID NO: 277 |
| CW795_2 | SEQ ID NO: 278 |
| CW823_3 | SEQ ID NO: 279 |
| DF989_3 | SEQ ID NO: 280 |
| DL162_1, DL162_2 | SEQ ID NO: 281 |
| EC172_1 | SEQ ID NO: 282 |

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:
(a) It should be designed to an area of the sequence which has the fewest ambiguous bases (“N's”), if any;
(b) It should be designed to have a Tₘ of approx. 80° C. (assuming 2° for each A or T and 4 degrees for each G or C).
   The oligonucleotide should preferably be labeled with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1e+6 dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114 9245-9253 (1992), both of which are incorporated herein by reference. Such fragments may be fused to carrier molecules such as immunoglobulins for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein—IgM fusion would generate a decavalent form of the protein of the invention.

The present invention also provides both full-length and mature forms of the disclosed proteins. The full-length form of the such proteins is identified in the sequence listing by translation of the nucleotide sequence of each disclosed clone. The mature form(s) of such protein may be obtained by expression of the disclosed full-length polynucleotide (preferably those deposited with the ATCC) in a suitable mammalian cell or other host cell. The sequence(s) of the mature form(s) of the protein may also be determinable from the amino acid sequence of the full-length form.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

The chromosomal location corresponding to the polynucleotide sequences disclosed herein may also be determined, for example by hybridizing appropriately labeled polynucleotides of the present invention to chromosomes in situ. It may also be possible to determine the corresponding chromosomal location for a disclosed polynucleotide by identifying significantly similar nucleotide sequences in public databases, such as expressed sequence tags (ESTs), that have already been mapped to particular chromosomal locations. For at least some of the polynucleotide sequences disclosed herein, public database sequences having at least some similarity to the polynucleotide of the present invention have been listed by database accession number. Searches using the GenBank accession numbers of these public database sequences can then be performed at an Internet site provided by the National Center for Biotechnology Information having the address http://www.ncbi nlm.nih.gov/UniGene/, in order to identify “UniGene clusters” of overlapping sequences. Many of the “UniGene clusters” so identified will already have been mapped to particular chromosomal sites.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). The desired change in gene expression can also be achieved through the use of double-stranded ribonucleotide molecules having some complementarity to the mRNA transcribed from the gene, and which interfere with the transcription, stability, or expression of the mRNA (“RNA intereference” or “RNAi”; Fire et al., 1998, Nature 391 (6669): 806-811; Montgomery et al., 1998, Proc. Natl. Acad. Sci. USA 95 (26): 15502-15507; and Sharp, 1999, Genes Dev. 13 (2): 139-141; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein).

In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms, part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information. For example, the TopPredII computer program can be used to predict the location of transmembrane domains in an amino acid sequence, domains which are described by the location of the center of the transmsmbrane domain, with at least ten transmembrane amino acids on each side of the reported central residue(s). Proteins and protein fragments of the present invention include proteins with amino acid sequence lengths that are at least 25% (more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

In particular, sequence identity may be determined using WU-BLAST (Washington University BLAST) version 2.0 software, which builds upon WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein). WU-BLAST version 2.0 executable programs for several UNIX platforms can be downloaded from ftp://blast.wustl.edu/blast/executables. The complete suite of search programs (BLASTP, BLASTN, BLASTX, TBLASTN, and TBLASTX) is provided at that site, in addition to several support programs. WU-BLAST 2.0 is copyrighted and may not be sold or redistributed in any form or manner without the express written consent of the author; but the posted executables may otherwise be freely used for commercial, nonprofit, or academic purposes. In all search programs in the suite—BLASTP, BLASTN, BLASTX, TBLASTN and TBLASTX—the gapped alignment routines are integral to the database search itself, and thus yield much better sensitivity and selectivity while producing the more easily interpreted output. Gapping can optionally be turned off in all of these programs, if desired. The default penalty (Q) for a gap of length one is Q=9 for proteins and BLASTP, and Q=10 for BLASTN, but may be changed to any integer value including zero, one through eight, nine, ten, eleven, twelve through twenty, twenty-one through fifty, fifty-one through one hundred, etc. The default per-residue penalty for extending a gap (R) is R=2 for proteins and BLASTP, and R=10 for BLASTN, but may be changed to any integer value including zero, one, two, three, four, five, six, seven, eight, nine, ten, eleven, twelve through twenty, twenty-one through fifty, fifty-one through one hundred, etc. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Species homologues of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulus griseus, Felis catus, Mustela vison, Canis familiaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuánez, 1988, Ann. Rev. Genet. 22: 323-351; O'Brien et al., 1993, Nature Genetics 3:103-112; Johansson et al., 1995, Genomics 25: 682-690; Lyons et al., 1997, Nature Genetics 15:47-56; O'Brien et al., 1997, Trends in Genetics 13(10): 393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein. The present invention also includes polynucleotides that hybridize under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

| | | Hybrid | | Wash |
|---|---|---|---|---|
| Stringency | Polynucleotide | Length | Hybridization Temperature and | Temperature |
| Condition | Hybrid | (bp)^{‡} | Buffer^{†} | and Buffer^{†} |
| A | DNA:DNA | ≧50 | 65° C.; 1 × SSC -or- | 65° C.; 0.3 × SSC |
| | | | 42° C.; 1 × SSC, 50% formamide | |
| B | DNA:DNA | <50 | T_{B}*; 1 × SSC | T_{B}*; 1 × SSC |
| C | DNA:RNA | ≧50 | 67° C.; 1 × SSC -or- | 67° C.; 0.3 × SSC |
| | | | 45° C.; 1 × SSC, 50% formarnide | |
| D | DNA:RNA | <50 | T_{D}*; 1 × SSC | T_{D}*; 1 × SSC |
| E | RNA:RNA | ≧50 | 70° C.; 1 × SSC -or- | 70° C.; 0.3 × SSC |
| | | | 50° C.; 1 × SSC, 50% formamide | |
| F | RNA:RNA | <50 | T_{F}*; 1 × SSC | T_{F}*; 1 × SSC |
| G | DNA:DNA | ≧50 | 65° C.; 4 × SSC -or- | 65° C.; 1 × SSC |
| | | | 42° C.; 4 × SSC, 50% formamide | |
| H | DNA:DNA | <50 | T_{H}*; 4 × SSC | T_{H}*; 4 × SSC |
| I | DNA:RNA | ≧50 | 67° C.; 4 × SSC -or- | 67° C.; 1 × SSC |
| | | | 45° C.; 4 × SSC, 50% formamide | |
| J | DNA:RNA | <50 | T_{J}*; 4 × SSC | T_{J}*; 4 × SSC |
| K | RNA:RNA | ≧50 | 70° C.; 4 × SSC -or- | 67° C.; 1 × SSC |
| | | | 50° C.; 4 × SSC, 50% formamide | |
| L | RNA:RNA | <50 | T_{L}*; 2 × SSC | T_{L}*; 2 × SSC |
| M | DNA:DNA | ≧50 | 50° C.; 4 × SSC -or- | 50° C.; 2 × SSC |
| | | | 40° C.; 6 × SSC, 50% formamide | |
| N | DNA:DNA | <50 | T_{N}*; 6 × SSC | T_{N}*; 6 × SSC |
| O | DNA:RNA | ≧50 | 55° C.; 4 × SSC -or- | 55° C.; 2 × SSC |
| | | | 42° C.; 6 × SSC, 50% formamide | |
| P | DNA:RNA | <50 | T_{P}*; 6 × SSC | T_{P}*; 6 × SSC |
| Q | RNA:RNA | ≧50 | 60° C.; 4 × SSC -or- | 60° C.; 2 × SSC |
| | | | 45° C.; 6 × SSC, 50% formamide | |
| R | RNA:RNA | <50 | T_{R}*; 4 × SSC | T_{R}*; 4 × SSC |
| ^{‡}The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{†}SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

The isolated polynucleotide endcoing the protein of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLabs (Beverly, Mass.), Pharmacia (Piscataway, N.J.) and Invitrogen Corporation (Carlsbad, Calif.), respectively. The protein can also be tagged with an epitope and subsequently purified by using a specific antibody directed to such epitope. One such epitope (“Flag”) is commercially available from the Eastman Kodak Company (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Uses and Biological Activity

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, those described in Gyuris et al., 1993, Cell 75: 791-803 and in Rossi et al., 1997, Proc. Natl. Acad. Sci. USA 94: 8405-8410, all of which are incorporated by reference herein) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor-dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A.M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e. a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J. E. e. a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e. a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6—Nordan, R. In Current Protocols in Immunology. J. E. e. a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11—Bennett, F., Giannotti, J., Clark, S. C. and Turner, K. J. In Current Protocols in Immunology. J. E. e. a. Coligan eds. Vol 1 pp. 6.15.1 John Wiley and Sons, Toronto. 1991; Measurement of mouse and human Interleukin 9—Ciarletta, A., Giannotti, J., Clark, S. C. and Turner, K. J. In Current Protocols in Immunology. J. E. e. a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A.M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course; in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to regulate immune responses in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II a chain protein and an MHC class II P chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol.128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. e. a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265_{—}268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention-may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing pps. 71-112 (Maibach, H I and Rovee, D T, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59_{—}68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Cadherin/Tumor Invasion Suppressor Activity

Cadherins are calcium-dependent adhesion molecules that appear to play major roles during development, particularly in defining specific cell types. Loss or alteration of normal cadherin expression can lead to changes in cell adhesion properties linked to tumor growth and metastasis. Cadherin malfunction is also implicated in other human diseases, such as pemphigus vulgaris and pemphigus foliaceus (auto-immune blistering skin diseases), Crohn's disease, and some developmental abnormalities.

The cadherin superfamily includes well over forty members, each with a distinct pattern of expression. All members of the superfamily have in common conserved extracellular repeats (cadherin domains), but structural differences are found in other parts of the molecule. The cadherin domains bind calcium to form their tertiary structure and thus calcium is required to mediate their adhesion. Only a few amino acids in the first cadherin domain provide the basis for homophilic adhesion; modification of this recognition site can change the specificity of a cadherin so that instead of recognizing only itself, the mutant molecule can now also bind to a different cadherin. In addition, some cadherins engage in heterophilic adhesion with other cadherins.

E-cadherin, one member of the cadherin superfamily, is expressed in epithelial cell types. Pathologically, if E-cadherin expression is lost in a tumor, the malignant cells become invasive and the cancer metastasizes. Transfection of cancer cell lines with polynucleotides expressing E-cadherin has reversed cancer-associated changes by returning altered cell shapes to normal, restoring cells' adhesiveness to each other and to their substrate, decreasing the cell growth rate, and drastically reducing anchorage-independent cell growth. Thus, reintroducing E-cadherin expression reverts carcinomas to a less advanced stage. It is likely that other cadherins have the same invasion suppressor role in carcinomas derived from other tissue types. Therefore, proteins of the present invention with cadherin activity, and polynucleotides of the present invention encoding such proteins, can be used to treat cancer. Introducing such proteins or polynucleotides into cancer cells can reduce or eliminate the cancerous changes observed in these cells by providing normal cadherin expression.

Cancer cells have also been shown to express cadherins of a different tissue type than their origin, thus allowing these cells to invade and metastasize in a different tissue in the body. Proteins of the present invention with cadherin activity, and polynucleotides of the present invention encoding such proteins, can be substituted in these cells for the inappropriately expressed cadherins, restoring normal cell adhesive properties and reducing or eliminating the tendency of the cells to metastasize.

Additionally, proteins of the present invention with cadherin activity, and polynucleotides of the present invention encoding such proteins, can used to generate antibodies recognizing and binding to cadherins. Such antibodies can be used to block the adhesion of inappropriately expressed tumor-cell cadherins, preventing the cells from forming a tumor elsewhere. Such an anti-cadherin antibody can also be used as a marker for the grade, pathological type, and prognosis of a cancer, i.e. the more progressed the cancer, the less cadherin expression there will be, and this decrease in cadherin expression can be detected by the use of a cadherin-binding antibody.

Fragments of proteins of the present invention with cadherin activity, preferably a polypeptide comprising a decapeptide of the cadherin recognition site, and poly-nucleotides of the present invention encoding such protein fragments, can also be used to block cadherin function by binding to cadherins and preventing them from binding in ways that produce undesirable effects. Additionally, fragments of proteins of the present invention with cadherin activity, preferably truncated soluble cadherin fragments which have been found to be stable in the circulation of cancer patients, and polynucleotides encoding such protein fragments, can be used to disturb proper cell-cell adhesion.

Assays for cadherin adhesive and invasive suppressor activity include, without limitation, those described in: Hortsch et al. J Biol Chem 270 (32): 18809-18817, 1995; Miyaki et al. Oncogene 11: 2547-2552, 1995; Ozawa et al. Cell 63: 1033-1038, 1990.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via antibody-dependent cell-mediated cytotoxicity (ADCC)). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL4, IL-5, IL6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. The pharmaceutical composition may further contain other agents which either enhance the activity of the protein or compliment its activity or use in treatment Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 ng to about 10 mg, more preferably about 0.1 μg to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Protein of the invention may also be used to immunize animals to obtain polyclonal and monoclonal antibodies which specifically react with the protein. As used herein, the term “antibody” includes without limitation a polyclonal antibody, a monoclonal antibody, a chimeric antibody, a single-chain antibody, a CDR-grafted antibody, a humanized antibody, or fragments thereof which bind to the indicated protein. Such term also includes any other species derived from an antibody or antibody sequence which is capable of binding the indicated protein.

Antibodies to a particular protein can be produced by methods well known to those skilled in the art. For example, monoclonal antibodies can be produced by generation of antibody-producing hybridomas in accordance with known methods (see for example, Goding, 1983, Monoclonal antibodies: principles and practice, Academic Press Inc., New York; and Yokoyama, 1992, “Production of Monoclonal Antibodies” in Current Protocols in Immunology, Unit 2.5, Greene Publishing Assoc. and John Wiley & Sons). Polyclonal sera and antibodies can be produced by inoculation of a mammalian subject with the relevant protein or fragments thereof in accordance with known methods. Fragments of antibodies, receptors, or other reactive peptides can be produced from the corresponding antibodies by cleavage of and collection of the desired fragments in accordance with known methods (see for example, Goding, supra; and Andrew et al., 1992, “Fragmentation of Immunoglobulins” in Current Protocols in Immunology, Unit 2.8, Greene Publishing Assoc. and John Wiley & Sons). Chimeric antibodies and single chain antibodies can also be produced in accordance with known recombinant methods (see for example, U.S. Pat. Nos. 5,169,939, 5,194,594, and 5,576,184). Humanized antibodies can also be made from corresponding murine antibodies in accordance with well known methods (see for example, U.S. Pat. Nos. 5,530,101, 5,585,089, and 5,693,762). Additionally, human antibodies may be produced in non-human animals such as mice that have been genetically altered to express human antibody molecules (see for example Fishwild et al., 1996, Nature Biotechnology 14: 845-851; Mendez et al., 1997, Nature Genetics 15: 146-156 (erratum Nature Genetics 16: 410); and U.S. Pat. Nos. 5,877,397 and 5,625,126). Such antibodies maybe obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987).

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 020 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells.

In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins of the present invention.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA).

Cells may also be cultured ex vivo in the presence of proteins of the present invention in order to proliferate or to produce a desired effect on or activity in such cells. Treated cells can then be introduced in vivo for therapeutic purposes.

Patent and literature references cited herein are incorporated by reference as if fully set forth.

1-13. (canceled)

14. A composition comprising an isolated polynucleotide selected from the group consisting of:
(a) a polynucleotide comprising the nucleotide sequence of SEQ ID NO:34;
(b) a polynucleotide comprising the nucleotide sequence of SEQ ID NO:34 from nucleotide 437 to nucleotide 1159;
(c) a polynucleotide comprising the nucleotide sequence of SEQ ID NO:34 from nucleotide 515 to nucleotide 1159;
(d) a polynucleotide comprising the nucleotide sequence of SEQ ID NO:34 from nucleotide 539 to nucleotide 1099;
(e) a polynucleotide comprising the nucleotide sequence of the full-length protein coding sequence of clone AR415_{—}4 deposited with the ATCC under accession number 98232;
(f) a polynucleotide comprising a sequence encoding the full-length protein encoded by the cDNA insert of clone AR415_{—}4 deposited with the ATCC under accession number 98232;
(g) a polynucleotide comprising the nucleotide sequence of a mature protein coding sequence of clone AR415_{—}4 deposited with the ATCC under accession number 98232;
(h) a polynucleotide encoding a mature protein encoded by the cDNA insert of clone AR415_{—}4 deposited with the ATCC under accession number 98232;
(i) a polynucleotide encoding a protein comprising the amino acid sequence of SEQ ID NO:35; and
(j) a polynucleotide encoding a protein comprising a fragment of the amino acid sequence of SEQ ID NO:35 having biological activity, the fragment comprising eight contiguous amino acids of SEQ ID NO:35.

15. The composition comprising a polynucleotide according to claim 14, comprising the nucleotide sequence of SEQ ID NO:34 from nucleotide 437 to nucleotide 1159.

16. The composition comprising a polynucleotide according to claim 14, comprising the nucleotide sequence of SEQ ID NO:34 from nucleotide 515 to nucleotide 1159.

17. The composition comprising a polynucleotide according to claim 14, comprising the nucleotide sequence of SEQ ID NO:34 from nucleotide 539 to nucleotide 1099.

18. The composition comprising a polynucleotide according to claim 14, comprising the nucleotide sequence of the full-length protein coding sequence of clone AR415_{—}4 deposited with the ATCC under accession number 98232.

19. The composition comprising a polynucleotide according to claim 14, comprising the nucleotide sequence of a mature protein coding sequence of clone AR415_{—}4 deposited with the ATCC under accession number 98232.

20. The composition comprising a polynucleotide according to claim 14, comprising the full-length or a mature protein encoded by the cDNA insert of clone AR415_{—}4 deposited with the ATCC under accession number 98232.

21. The composition according to claim 14, comprising a polynucleotide comprising a nucleotide sequence encoding a protein comprising the amino acid sequence of SEQ ID NO:35 from amino acid 51 to amino acid 221.

22. The composition according to claim 14, comprising a polynucleotide comprising a nucleotide sequence encoding a protein comprising a fragment of the amino acid sequence of SEQ ID NO:35 having biological activity,
wherein the fragment preferably comprising eight contiguous amino acids of SEQ ID NO:35.

23. The composition according to claim 14, comprising a polynucleotide comprising a nucleotide sequence encoding a protein comprising a fragment of the amino acid sequence of SEQ ID NO:35 having biological activity,
wherein the fragment comprising the amino acid sequence from amino acid 115 to amino acid 124 of SEQ ID NO:35.

24. An isolated polynucleotide produced according to a process selected from the group consisting of:
(a) a process comprising the steps of:
(i) preparing one or more polynucleotide probes that hybridize in 6×SSC at 65 degrees C. to a nucleotide sequence selected from the group consisting of:
(aa) SEQ ID NO:34, but excluding the poly(A) tail at the 3′ end of SEQ ID NO:34; and
(ab) the nucleotide sequence of the cDNA insert of clone AR415_{—}4 deposited with the ATCC under accession number 98232;
(ii) hybridizing said probe(s) to human genomic DNA in conditions at least as stringent as 4×SSC at 50 degrees C.; and
(iii) isolating the DNA polynucleotides detected with the probe(s); and
(b) a process comprising the steps of:
(i) preparing one or more polynucleotide primers that hybridize in 6×SSC at 65 degrees C. to a nucleotide sequence selected from the group consisting of:
(ba) SEQ ID NO:34, but excluding the poly(A) tail at the 3′ end of SEQ ID NO:34; and
(bb) the nucleotide sequence of the cDNA insert of clone AR415_{—}4 deposited with the ATCC under accession number 98232;
(ii) hybridizing said primer(s) to human genomic DNA in conditions at least as stringent as 4×SSC at 50 degrees C.;
(iii) amplifying human DNA sequences; and
(iv) isolating the polynucleotide products of step (b)(iii).

25. An isolated polynucleotide isolated according claim 24 comprises a nucleotide sequence corresponding to the cDNA sequence of SEQ ID NO:34, and extending contiguously from a nucleotide sequence corresponding to the 5′ end of SEQ ID NO:34 to a nucleotide sequence corresponding to the 3′ end of SEQ ID NO:34, but excluding the poly(A) tail at the 3′ end of SEQ ID NO:34.

26. An isolated polynucleotide according claim 24, comprising a nucleotide sequence corresponding to the cDNA sequence of SEQ ID NO:34 from nucleotide 437 to nucleotide 1159, and extending contiguously from a nucleotide sequence corresponding to the 5′ end of said sequence of SEQ ID NO:34 from nucleotide 437 to nucleotide 1159, to a nucleotide sequence corresponding to the 3′ end of said sequence of SEQ ID NO:34 from nucleotide 437 to nucleotide 1159.

27. An isolate polynucleotide according to claim 24, comprising a nucleotide sequence corresponding to the cDNA sequence of SEQ ID NO:34 from nucleotide 515 to nucleotide 1159, and extending contiguously from a nucleotide sequence corresponding to the 5′ end of said sequence of SEQ ID NO:34 from nucleotide 515 to nucleotide 1159, to a nucleotide sequence corresponding to the 3′ end of said sequence of SEQ ID NO:34 from nucleotide 515 to nucleotide 1159.

28. An isolate polynucleotide according to claim 24, comprising a nucleotide sequence corresponding to the cDNA sequence of SEQ ID NO:34 from nucleotide 539 to nucleotide 1099, and extending contiguously from a nucleotide sequence corresponding to the 5′ end of said sequence of SEQ ID NO:34 from nucleotide 539 to nucleotide 1099, to a nucleotide sequence corresponding to the 3′ end of said sequence of SEQ ID NO:34 from nucleotide 539 to nucleotide 1099.

29. A composition comprising a protein, comprising an amino acid sequence selected from the group consisting of:
(a) the amino acid sequence of SEQ ID NO:35;
(b) the amino acid sequence of SEQ ID NO:35 from amino acid 51 to amino acid 221;
(c) a fragment of the amino acid sequence of SEQ ID NO:35, the fragment comprising eight contiguous amino acids of SEQ ID NO:35; and
(d) the amino acid sequence encoded by the cDNA insert of clone AR415_{—}4 deposited with the ATCC under accession number 98232;
wherein the protein being substantially free from other mammalian proteins.

30. The protein according to claim 29, comprising the amino acid sequence of SEQ ID NO:35 or the amino acid sequence of SEQ ID NO:35 from amino acid 51 to amino acid 221.

31. The protein according to claim 29, comprising the present invention provides a protein comprising a fragment of the amino acid sequence of SEQ ID NO:35 having biological activity, the fragment preferably comprising eight contiguous amino acids of SEQ ID NO:35

32. The protein according to claim 29, comprising a fragment of the amino acid sequence of SEQ ID NO:35 having biological activity, the fragment comprising the amino acid sequence from amino acid 115 to amino acid 124 of SEQ ID NO:35.
